SEQUENCE LISTING (12 sequences)

SEQ ID NO: 1 - DNA, 9674 bp, synthetic construct
  misc_feature 1..9674: note = Description of Artificial Sequence: Syntheticpolynucleotide
  source 1..9674: mol_type = other DNA
aaccatctcgcaaataaataagtattttactgttttcgtaacagttttgtaataaaaaaacctataaatattccggattattcataccgtcccaccatcgggcgccatggcttctcaccaccatcaccatcaccatcatcatcacgctcagcacgacgaggctgtggacaacaagttcaacaaggagcagcagaacgctttctacgagatcctgcacctccctaacctgaacgaggagcagcgtaacgctttcatccagtccctgaaggacgacccttctcagtctgctaacctgctggctgaggctaagaagctgaacgacgctcaggctcctaaggtggacaacaagttcaacaaggagcagcagaacgctttctacgagatcctgcacctccctaacctgaacgaggagcagcgtaacgctttcatccagtccctgaaggacgacccttctcagtctgctaacctgctggctgaggctaagaagctgaacgacgctcaggctcctaaggtggacgctaacggtggcggcggttccggcggtggtggctctctcgaggttctgttccagggtccgaatgaaggcgaggaagatgatgacaaagacttcctgtggccagctccaaacgaagaacaggtgacttgtctcaagatgtacttcggtcatagcagcttcaaaccagtgcagtggaaagttatccacagcgttcttgaagaacgtcgtgataatgtggctgtgatggctactggctatggtaagagcctgtgtttccagtacccgccagtttacgttggtaagatcggtctggtgattagcccgctgatctctctgatggaagaccaggtgctgcaacttaagatgagcaacatcccggcttgtttcctgggttctgcacaaagcgagaacgtgctcaccgatatcaagctgggtaagtaccgtatcgtgtacgtgacgccagaatactgtagcggcaacatgggtcttctgcaacagctcgaagctgatattggcatcaccctcattgcagtggacgaagctcactgtatcagcgagtggggtcatgatttccgcgactctttccgtaaactgggttctctgaagactgcacttccgatggttccaattgtggcactgaccgcaactgcttctagctctattcgtgaagacatcgttcgttgcctgaacctccgtaacccacaaattacctgcaccggctttgaccgtccgaacctgtacctggaggttcgtcgtaagaccggtaatatccttcaggacctgcaaccattcctggttaagaccagcagccactgggagttcgaaggtccgactatcatctactgcccaagccgtaagatgacccagcaggttactggtgaactgcgtaaactgaacctgagctgtggcacttaccacgcaggcatgtctttctctacccgtaaagacatccatcatcgtttcgtgcgtgatgaaatccagtgcgttatcgctaccattgcattcggcatgggtatcaacaaagctgacatccgtcaagtgattcactacggtgcaccgaaagacatggaaagctactaccaggaaatcggccgtgcaggtcgtgatggtctgcaaagctcttgtcatgtgctgtgggcaccagcagatattaacctgaaccgtcacctgctgactgaaattcgtaacgagaaattccgtctgtacaaactgaagatgatggcgaagatggagaaatacctgcatagctcccgttgtcgtcgtcaaatcattctgagccatttcgaggataaacaggtgcagaaagcttctctgggtatcatgggcactgagaagtgctgcgataactgtcgtagccgtcttgatcactgctacagcatggacgatagcgaagacacttcttgggatttcggtccacaagcattcaaactgctgagcgcagttgatatcctgggtgagaaattcggcatcggcctcccaatcctgtttctgcgcggttctaactctcagcgtcttgctgatcaataccgtcgtcactctctgttcggcactggtaaagaccagaccgaatcttggtggaaagcattcagccgtcaacttatcaccgaaggctttctggtggaagtgtctcgttacaacaagttcatgaagatctgcgcactgactaagaaaggtcgtaactggctgcacaaggcaaataccgagtctcagtctcttatccttcaggctaacgaagaactgtgcccgaagaagcttctgctgccatcttctaagaccgtgagctctggtactaaagagcattgctacaaccaggtgccggttgaactgtctaccgagaagaagtccaacctggagaagctgtactcctacaaaccgtgcgacaagatctcctccggttctaatatcagcaagaagtccatcatggtgcagtctccggagaaagcttacagcagctctcagccagttatctctgcacaggaacaggaaactcagattgtgctgtacggtaaactggtggaagcacgtcagaaacacgctaacaagatggacgtgccgccagcaattcttgcaaccaacaagattctggtggacatggctaagatgcgcccaactactgttgagaacgtgaaacgtatcgacggtgttagcgaaggtaaagctgcaatgctggcaccactgcttgaagttatcaagcatttctgccagaccaactctgttcagaccgacctgttctcttctaccaaaccataatggtaccgaattcgcggccgcagagctcgctctggtgccacgcggtagttccgcttggagccacccgcagttcgaaaagtaagtgattaacctcaggttatacatatattttgaatttaattaattatacatatattttatattatttttgtcttttattatcgaggggccgttgttggtgtggggttttgcatagaaataacaatgggagttggcgacgttgctgcgccaacaccacctcccttccctcctttcatcatgtatctgtagataaaataaaatattaaacctaaaaacaagaccgcgcctatcaacaaaatgataggcattaacttgccgctgacgctgtcactaacgttggacgatttgccgactaaaccttcatcgcccagtaaccaatctaggtagctgagcgcatgcaagctgatccgggttattagtacatttattaagcgctagattctgtgcgttgttgatttacagacaattgttgtacgtattttaataattcattaaatttataatctttagggtggtatgttagagcgaaaatcaaatgattttcagcgtctttatatctgaatttaaatattaaatcctcaatagatttgtaaaataggtttcgattagtttcaaacaagggttgtttttccgaaccgatggctggactatctaatggattttcgctcaacgccacaaaacttgccaaatcttgtagcagcaatctagctttgtcgatattcgtttgtgttttgttttgtaataaaggttcgacgtcgttcaaaatattatgcgcttttgtatttctttcatcactgtcgttagtgtacaattgactcgacgtaaacacgttaaatagagcttggacatatttaacatcgggcgtgttagctttattaggccgattatcgtcgtcgtcccaaccctcgtcgttagaagttgcttccgaagacgattttgccatagccacacgacgcctattaattgtgtcggctaacacgtccgcgatcaaatttgtagttgagctttttggaattatttctgattgcgggcgtttttgggcgggtttcaatctaactgtgcccgattttaattcagacaacacgttagaaagcgatggtgcaggcggtggtaacatttcagacggcaaatctactaatggcggcggtggtggagctgatgataaatctaccatcggtggaggcgcaggcggggctggcggcggaggcggaggcggaggtggtggcggtgatgcagacggcggtttaggctcaaatgtctctttaggcaacacagtcggcacctcaactattgtactggtttcgggcgccgtttttggtttgaccggtctaagacgagtgcgatttttttcgtttctaatagcttccaacaattgttgtctgtcgtctaaaggtgcagcgggttgaggttccgtcggcattggtggagcgggcggcaattcagacatcgatggtggtggtggtggtggaggcgctggaatgttaggcacgggagaaggtggtggcggcggtgccgccggtataatttgttctggtttagtttgttcgcgcacgattgtgggcaccggcgcaggcgccgctggctgcacaacggaaggtcgtctgcttcgaggcagcgcttggggtggtggcaattcaatattataattggaatacaaatcgtaaaaatctgctataagcattgtaatttcgctatcgtttaccgtgccgatatttaacaaccgctcaatgtaagcaattgtattgtaaagagattgtctcaagctcggatcgatcccgcacgccgataacaagccttttcatttttactacagcattgtagtggcgagacacttcgctgtcgtcgaggtttaaacgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccattcgccattcaggctgcgcaactgttgggaagggcgatcggtgcgggcctcttcgctattacgccaggaacggctccgcccactattaatgaaattaaaaattccaattttaaaaaacgcagcaagagaaacatttgtatgaaagaatgcgtagaaggaaagaaaaatgtcgtcgacatgctgaacaacaagattaatatgcctccgtgtataaaaaaaatattgaacgatttgaaagaaaacaatgtaccgcgcggcggtatgtacaggaagaggtttatactaaactgttacattgcaaacgtggtttcgtgtgccaagtgtgaaaaccgatgtttaatcaaggctctgacgcatttctacaaccacgactccaagtgtgtgggtgaagtcatgcatcttttaatcaaatcccaagatgtgtataaaccaccaaactgccaaaaaatgaaaactgtcgacaagctctgtccgtttgctggcaactgcaagggtctcaatcctatttgtaattattgaataataaaacaattataaatgctaaatttgttttttattaacgatacaaaccaaacgcaacaagaacatttgtagtattatctataattgaaaacgcgtagttataatcgctgaggtaatatttaaaatcattttcaaatgattcacagttaatttgcgacaatataattttattttcacataaactagacgccttgtcgtcttcttcttcgtattccttctctttttcatttttctcttcataaaaattaacatagttattatcgtatccatatatgtatctatcgtatagagtaaattttttgttgtcataaatatatatgtcttttttaatggggtgtatagtaccgctgcgcatagtttttctgtaatttacaacagtgctattttctggtagttcttcggagtgtgttgctttaattattaaatttatataatcaatgaatttgggatcgtcggttttgtacaatatgttgccggcatagtacgcagcttcttctagttcaattacaccattttttagcagcaccggattaacataactttccaaaatgttgtacgaaccgttaaacaaaaacagttcacctcccttttctatactattgtctgcgagcagttgtttgttgttaaaaataacagccattgtaataagacgcacaaactaatatcacaaactggaaatgtctatcaatatatagttgctgatcagatctacccgtagtggctatggcagggcttgccgccccgacgttggctgcgagccctgggccttcacccgaacttgggggttggggtggggaaaaggaagaaacgcgggcgtattggtcccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccggtaccgcatgctatgcatcggccgctttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatcgtcgagatcccgggcgtttaaattgtgtaatttatgtagctgtaatttttaccttattaatattttttacgctttgcattcgacgactgaactcccaaatatatgtttaactcgtcttggtcgtttgaatttttgttgctgtgtttcctaatattttccatcaccttaaatatgttattgtaatcctcaatgttgaacttgcaattggacacggcatagttttccatagtcgtgtaaaacatggtattggctgcattgtaatacatccgactgagcgggtacggatctatgtgtttgagcagcctgttcaaaaactctgcatcgtcgcaaaacggaatttggtacccgggcgtatactccggaatattaatagatcatggagataattaaaatgat

SEQ ID NO: 2 - DNA, 45 bp, synthetic construct
  misc_feature 1..45: note = Description of Artificial Sequence: Syntheticoligonucleotide
  source 1..45: mol_type = other DNA
ttttttttttttttttttttttccaagtaaaacgacggccagtgc

SEQ ID NO: 3 - protein, 445 aa, synthetic construct
  REGION 1..445: note = Description of Artificial Sequence: Syntheticpolypeptide
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 4 - protein, 214 aa, synthetic construct
  REGION 1..214: note = Description of Artificial Sequence: Syntheticpolypeptide
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 118 aa, synthetic construct
  REGION 1..118: note = Description of Artificial Sequence: Syntheticpolypeptide
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 6 - protein, 107 aa, synthetic construct
  REGION 1..107: note = Description of Artificial Sequence: Syntheticpolypeptide
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 7 - protein, 10 aa, synthetic construct
  REGION 1..10: note = Description of Artificial Sequence: Syntheticpeptide
GFSLTSYGVH

SEQ ID NO: 8 - protein, 16 aa, synthetic construct
  REGION 1..16: note = Description of Artificial Sequence: Syntheticpeptide
VIYADGSTNYNPSLKS

SEQ ID NO: 9 - protein, 12 aa, synthetic construct
  REGION 1..12: note = Description of Artificial Sequence: Syntheticpeptide
ARAYGNYWYIDV

SEQ ID NO: 10 - protein, 11 aa, synthetic construct
  REGION 1..11: note = Description of Artificial Sequence: Syntheticpeptide
KSSESVSNDVA

SEQ ID NO: 11 - protein, 7 aa, synthetic construct
  REGION 1..7: note = Description of Artificial Sequence: Syntheticpeptide
YAFHRFT

SEQ ID NO: 12 - protein, 9 aa, synthetic construct
  REGION 1..9: note = Description of Artificial Sequence: Syntheticpeptide
HQAYSSPYT

(19) United States
(12) Patent Application Publication
(10) Pub. No.: US 2024/0245694 A1
(43) Pub. Date: Jul. 25, 2024

(54) Tricyclic Compounds and their Uses

(71) Applicant: Novartis AG, Basel (CH)

(72) Inventors: Vincent BORDAS, Village-Neuf (FR); Markus FUREGATI, Allschwil (CH); Jacques HAMON, Habsheim (FR); Jürgen Hans-Hermann HINRICHS, Schopfheim (DE); Ziyue HONG, Shanghai (CN); Fabio LIMA, Saint Louis (FR); Fatma LIMAM, Strasbourg (FR); Henrik MOEBITZ, Arlesheim (CH); Sandro NOCITO, Wölflinswil (CH); Niko SCHMIEDEBERG, Riehen (CH); Joseph SCHOEPFER, Riehen (CH); Ross Sinclair STRANG, Hagenthal le Bas (FR); Frédéric ZECRI, Brookline, MA (US); Huangchao YU, Suzhou (CN); Yong ZHANG, Suzhou (CN); Xinkan YANG, Suzhou (CN); Sisi ZHANG, Suzhou (CN); Wei LI, Suzhou (CN)

(21) Appl. No.: 18/483,651

(22) Filed: Oct. 10, 2023

Related U.S. Application Data
(60) Provisional application No. 63/494,033, filed on Apr. 4, 2023.

(30) Foreign Application Priority Data
Oct. 12, 2022 (WO) PCT/CN2022/124865
Nov. 17, 2022 (EP) 22208192.9
Jul. 24, 2023 (WO) PCT/CN2023/108913

(51) Int. Cl. A61K 31/5377; A61K 31/519; C07D 487/14; C07D 519/00

(52) U.S. Cl. CPC A61K 31/5377; A61K 31/519; C07D 487/14; C07D 519/00

(57) ABSTRACT

The present invention provides a compound, or a pharmaceutically acceptable salt thereof, of formula (I):

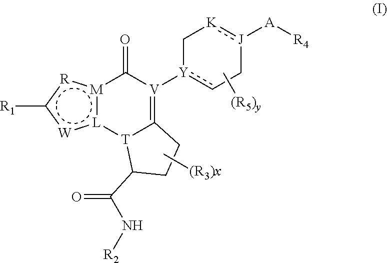

wherein R₁, R₂, R₃, x, R₄, R₅, y, R, M, W, L, V, T, Y, J, K and A are as described herein, therapeutic uses of said compounds, uses of said compounds as research chemicals, a pharmaceutical composition and combinations comprising said compounds, and methods for manufacturing the compounds of the invention.

### SEQUENCE LISTING

The instant application contains a Sequence Listing, which has been submitted electronically in XML format and is hereby incorporated by reference in its entirety. Said XML copy, created on Oct. 9, 2023, is named PAT059226-US-NP-Sequence Listing.xml and is 27,013 bytes in size.

### FIELD OF INVENTION

The invention provides tricyclic heterocyclic compounds, such as 5-oxo-5,7,8,9-tetrahydropyrrolo[1,2-c][1,2,4]triazolo[1,5-a]pyrimidine compounds and analogues and derivatives thereof, the use thereof for inhibiting Werner Syndrome RecQ DNA helicase (WRN) and methods of treating disease using said compounds, in particular the use in treating cancer, and in particular the treatment of cancer characterized as microsatellite instability-high (MSI-H) or mismatch repair deficient (dMMR), including colorectal, gastric and endometrial cancer. The invention also provides the use of said compounds as research chemicals, intermediate compounds, combinations, processes and formulations.

### BACKGROUND OF THE INVENTION

Loss of DNA mismatch repair is a common initiating event in cancer development occurring in 10-30% of colorectal, endometrial, ovarian and gastric cancers (Aaltonen, L. A. et al. Clues to the pathogenesis of familial colorectal cancer, Science 260, 812-816 (1993), Bonneville R et al., Landscape of Microsatellite Instability Across 39 Cancer Types. JCO Precis Oncol. 1: PO.17.00073 (2017)). Cancers that have lost competence in mismatch repair (MMR) have a high mutational burden, and frequent deletion and insertion events in repetitive DNA tracts, a phenotype known as microsatellite instability (MSI). While progress has been made in the treatment of microsatellite instability high (MSI-H) cancers, and the demonstration that pembrolizumab (anti-PD1) treatment led to significantly longer progression-free survival than chemotherapy when received as first-line therapy for MSI-H-dMMR metastatic colorectal cancer resulted in the recent approval of pembrolizumab as first-line treatment of these cancers, there is still a significant unmet medical need in CRC and other MSI-H indications (Andre T., et al. Pembrolizumab in Microsatellite-Instability-High Advanced Colorectal Cancer. N Engl J Med 383(23):2207-2218 (2020)). Several large-scale functional genomics screens across large panels of cell lines, including Novartis with 398 cell lines from the Cancer Cell Line Encyclopedia (CCLE) (McDonald E. R. et al., Project DRIVE: A Compendium of Cancer Dependencies and Synthetic Lethal Relationships Uncovered by Large-Scale, Deep RNAi Screening. Cell 170(3):577-592 (2017)), have identified the Werner Syndrome RecQ helicase (WRN) as being selectively required for the survival of cell lines with defective mismatch repair that have become MSI-H (Behan, F. M. et al. Prioritization of cancer therapeutic targets using CRISPR-Cas9 screens. Nature 568, 511-516 (2019), Chan, E. M. et al. WRN helicase is a synthetic lethal target in microsatellite unstable cancers. Nature 568, 551-556 (2019). Kategaya, L., Perumal, S. K., Hager, J. H. & Belmont, L. D. Werner syndrome helicase is required for the survival of cancer cells with microsatellite instability. iScience 13, 488-497 (2019), Lieb, S. et al. Werner syndrome helicase is a selective vulnerability of microsatellite instability-high tumor cells. eLife 8, e43333 (2019)). WRN is synthetic lethal with MSI cancers. Depletion of WRN leads to anti-proliferative effects and results in activation of multiple DNA damage signaling markers, induction of cell cycle arrest and apoptosis in MMR cancer models but not cancer cells with an intact MMR pathway. These findings indicate that WRN provides a DNA repair and maintenance function that is essential for cell survival in MSI cancers. Recently, the mechanism of WRN dependence has been elucidated. It has been shown that dinucleotide TA repeats are selectively unstable in MSI cells and undergo large scale expansions. These expanded TA repeats form secondary DNA structures that require the WRN helicase for unwinding (van Wietmarschen, N. et al. Repeat expansions confer WRN dependence in microsatellite-unstable cancers. Nature 586, 292-298, 2020). In the absence of WRN (or upon WRN helicase inhibition), expanded TA repeats in MSI cells are subject to nuclease cleavage and chromosome breakage. Thus, inhibiting the WRN helicase is an attractive strategy for the treatment of mismatch repair defective cancers.

### SUMMARY OF THE INVENTION

There remains a need for new treatments and therapies for the treatment of cancer, and in particular cancers characterized as microsatellite instability-high (MSI-H) or mismatch repair deficient (dMMR), including colorectal, gastric or endometrial cancer. The invention provides compounds, pharmaceutically acceptable salts thereof, pharmaceutical compositions thereof and combinations thereof, said compounds being inhibitors of Werner Syndrome RecQ DNA Helicase (WRN). The invention further provides methods of treating, preventing, or ameliorating a disease or condition, comprising administering to a subject in need thereof an effective amount of a WRN inhibitor. The invention also provides compounds, pharmaceutically acceptable salts thereof, pharmaceutical compositions thereof and combinations thereof, said compounds being useful for the treatment of cancer, in particular cancers characterized as microsatellite instability-high (MSI-H) or mismatch repair deficient (dMMR). Also provided are compounds that bind to, and/or inhibit WRN, and are therefore useful as research chemicals, e.g. as a chemical probe, and as tool compounds. For example, such use may be in the research of WRN related disease, or MSI high disorders.

Various embodiments of the invention are described herein.

Within certain aspects, provided herein is a compound of formula (I) or a pharmaceutically acceptable salt thereof:

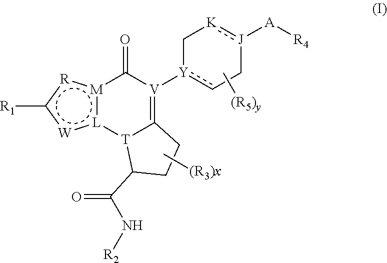

wherein
R, M, W, L, V and T are independently selected from C, CH and N,
to form subformulae 1a, 1b, 1c, 1d, 1e and 1f:

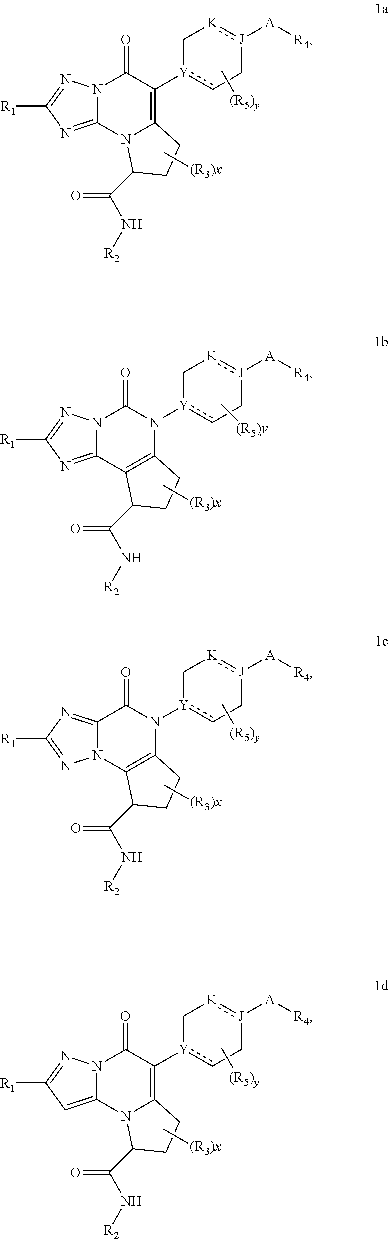

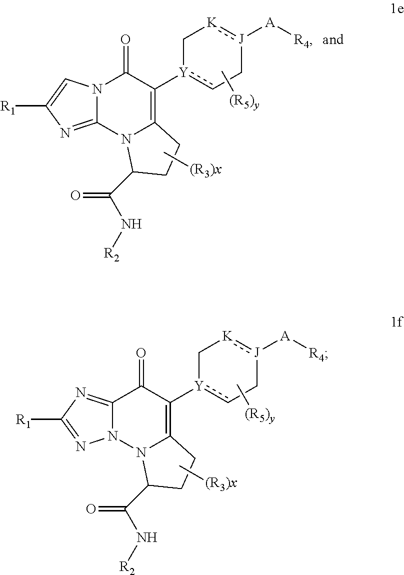

A is a linker selected from —C(O)—, —S(O)—, —S(O)₂—, and

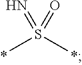

Y is N, C or CH;
Y [symbol] means Y is linked via a single bond to the adjacent carbon atom when Y is CH, or Y is linked via a double bond to the adjacent atom when Y is C, and when Y [symbol] is a single bond, Y is carbon unsubstituted or substituted by OH or F;
when Y is N, Y [symbol] is a single bond;
K [symbol] means K is linked via a single or double bond to the adjacent atom;
wherein:
   when K [symbol] is a double bond, Y [symbol] is a single bond, K is CH, J is C, and A is a linker selected from —C(O)—, —S(O)—, —S(O)₂—, and

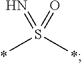

or
   when K [symbol] is a single bond, K is selected from —CH₂—, —CH₂CH₂—, —NH— and a bond (to form a 5-membered ring:

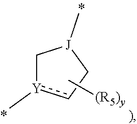

J is N, and A is a linker selected from —C(O)—, —S(O)—, —S(O)₂—, and

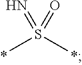

or
   when K [symbol] is a single bond, K is —CH₂—, J is CH, and A is a linker selected from —S(O)—, —S(O)₂—, and

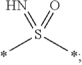

y is 0, 1, 2, 3 or 4;
R₅ is independently selected from:
   (C₁-C₄)alkyl,
   (C₃-C₅)cycloalkyl,
   and wherein two R₅ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S,
   when K [symbol] is a carbon-nitrogen single bond, a R₅ substituent on K and on the [symbol] adjacent carbon atom may join to form ring C:

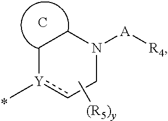

wherein ring C is a fused (C₃-C₆)cycloalkyl ring, a fused (C₃-C₆)heterocyclyl ring or a fused phenyl ring, wherein said fused (C₃-C₆)heterocyclyl ring contains ring carbon atoms and one ring heteroatom selected from O, N and S,
   and wherein when ring C is a fused (C₃-C₆)cycloalkyl ring, said fused (C₃-C₆)cycloalkyl ring is unsubstituted or substituted with 1 or 2 R₄₀ groups, wherein said R₄₀ is selected from:
      (C₁-C₂)alkyl, wherein each (C₁-C₂)alkyl is independently unsubstituted or substituted by OH or 1, 2 or 3 halo,
      halo, in particular F,
      or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
      or wherein two R₄₀ substituents on adjacent carbon atoms join together with the carbon atoms to which they are attached, to form a fused cyclopropyl ring;
when K [symbol] J is a carbon-carbon single bond, Y is N and Y [symbol] is a single bond, and A is a linker selected from —S(O)—, —S(O)₂—, and

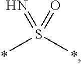

a R₅ substituent on K and on the adjacent carbon atom may join to form ring C:

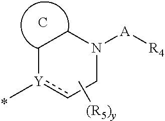

wherein ring C is a fused (C₃-C₆)cycloalkyl ring, a fused (C₃-C₆)heterocyclyl ring or a fused phenyl ring, wherein said fused (C₃-C₆)heterocyclyl ring contains ring carbon atoms and one ring heteroatom selected from O, N and S,
   and wherein when ring C is a fused (C₃-C₆)cycloalkyl ring, said fused (C₃-C₆)cycloalkyl ring is unsubstituted or substituted with 1 or 2 R₄₀ groups, wherein said R₄₀ is selected from:
      (C₁-C₂)alkyl, wherein each (C₁-C₂)alkyl is independently unsubstituted or substituted by OH or 1, 2 or 3 halo,
      halo, in particular F,
      or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
      or wherein two R₄₀ substituents on adjacent carbon atoms join together with the carbon atoms to which they are attached, to form a fused cyclopropyl ring;
and wherein when K is —CH₂— and J is N, two R₅ substituents may join to form a (C₁-C₃)alkylene bridge or a heteroalkylene bridge, wherein said heteroalkylene bridge is one heteroatom selected from N and O, or is —CH₂—O—CH₂—;
R₁ is:
cycloalkenyl, wherein said cycloalkenyl is a partially unsaturated monocyclic ring containing 5 or 6 ring carbon atoms, and said cycloalkenyl is unsubstituted or substituted by 1, 2, 3 or 4, preferably 1 or 2, R₃₃, wherein R₃₃ is halo, and wherein said cycloalkenyl or halo-substituted cycloalkenyl is substituted by 0, 1 or 2 R₁₅ substituents,
or R₁ is heterocyclyl, wherein said heterocyclyl is a 5 or 6 membered fully saturated or partially unsaturated group comprising ring carbon atoms and 1 or 2 ring heteroatoms independently selected from N, NH, O and S, and wherein said heterocyclyl is unbridged or bridged, and said bridge is 1 or 2 carbon atoms, wherein said heterocyclyl is unsubstituted or substituted by 1, 2, 3 or 4, preferably 1 or 2, R₃₃, wherein R₃₃ is halo, and wherein said heterocyclyl or halo-substituted heterocyclyl is substituted by 0, 1 or 2 substituents independently selected from R₁₅, R₁₆, R₁₇, R₁₈, R₁₉, R₂₀, R₂₂ and R₂₃,
or said heterocyclyl or halo-substituted heterocyclyl is fused to a cyclopropyl ring, wherein said cyclopropyl ring is unsubstituted or substituted by 1, 2 or 3 F,
or said heterocyclyl or halo-substituted heterocyclyl has 2 substituents at the same ring carbon atom which join to form a cyclopropyl spiro ring,
or said heterocyclyl or halo-substituted heterocyclyl is fused with a (C₃-C₅)heterocycloalkyl ring, wherein said (C₃-C₅)heterocycloalkyl ring contains ring carbon atoms and 1 ring O atom;
or R₁ is heteroaryl, wherein said heteroaryl is a 5 or 6 membered fully unsaturated monocyclic group comprising ring carbon atoms and 1, 2, 3 or 4 ring heteroatoms independently selected from N, O and S, preferably 1 or 2 ring heteroatoms, preferably wherein the total number of ring S atoms does not exceed 1, and the total number of ring O atoms does not exceed 1,
and wherein said heteroaryl is unsubstituted or substituted by 1, 2 or 3 substituents independently selected from R₂, and R₃₀, wherein R₂₁ and R₃₀ are independently selected from halo and (C₁-C₄)alkyl, wherein said (C₁-C₄)alkyl is unsubstituted or substituted by 1, 2 or 3 halo,
or R₁ is phenyl, wherein said phenyl is unsubstituted or substituted by 1, 2, 3 or 4, preferably 1 or 2, R₃₃, wherein R₃₃ is halo, and wherein said phenyl or halo-substituted phenyl is substituted by 0, 1 or 2 R₁₅ substituents,
or R₁ is (C₂-C₄)alkynyl or (C₂-C₄)alkenyl, wherein said (C₂-C₄)alkynyl and (C₂-C₄)alkenyl are unsubstituted or substituted by (C₁-C₄)alkyl-O—C(O)—, or morpholinyl;
each R₁₅, R₁₆, R₁₇, R₁₈, R₁₉, R₂₀, R₂₂ and R₂₃ is independently selected from:
   halo
   (C₁-C₄)alkyl-O— unsubstituted or substituted by 1, 2 or 3 halo;
   (C₁-C₄)alkyl unsubstituted or substituted by OH, —O—(C₁-C₂)alkyl or 1, 2 or 3 halo,
   HOC(O)—(CH₂)ₙ—,
   H₃C—C(O)(CH₂)ₙ—,
   (C₁-C₄)alkyl-O—C(O)(CH₂)ₙ,
   ═O
   azetidinyl or pyrrolidinyl, wherein said azetidinyl and pyrrolidinyl are linked to the rest of the molecule via the N atom, and are each unsubstituted or substituted by 1 or 2 F,
   R₂₅(R₂₄)N—, wherein R₂₄ is H or (C₁-C₄)alkyl unsubstituted or substituted by 1, 2 or 3 halo, R₂₅ is H or (C₁-C₄)alkyl unsubstituted or substituted by 1, 2 or 3 halo,
   OH
wherein n is 0, 1 or 2,
R₂ is the moiety:

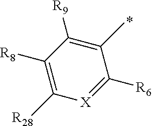

R₆ is selected from:
   H,
   halo,
   (C₁-C₄)alkyl unsubstituted or substituted by 1, 2 or 3 halo,
   (C₃-C₅)cycloalkyl unsubstituted or substituted by 1, 2 or 3 halo,
   ·-O—(C₁-C₄)alkyl unsubstituted or substituted by 1, 2 or 3 halo,
   OH, and
   CN;
R₈ is selected from H, halo, and (C₁-C₄)alkyl unsubstituted or substituted by 1, 2 or 3 halo,
R₉ is selected from H, O—CH₃, OH, CN, CH₃ and halo;
R₂₈ is selected from:
   SF₅,
   H,
   —C(O)H,
   halo,
   (C₁-C₄)alkyl unsubstituted or substituted by 1, 2 or 3 halo,
   (C₁-C₄)alkynyl,
   (C₁-C₄)alkenyl,
   (C₃-C₅)cycloalkyl unsubstituted or substituted by 1, 2 or 3 halo, and
   OCF₃;
X is selected from C—R₇ and N, wherein R₇ is H or halo, or R₇ can join, together with R₂₈ or R₆, and the atoms to which they are attached, to form a fused (C₄-C₆)cycloalkyl ring, wherein said fused (C₄-C₆)cycloalkyl ring is unsubstituted or substituted by 1, 2 or 3 halo,
or
R₂ is selected from:

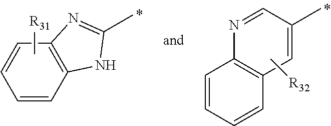

wherein
R₃₁ is selected from H, halo and CH₃,
R₃₂ is selected from H, halo and CH₃,
R₃ is selected from:
   halo, and
   (C₁-C₄)alkyl unsubstituted or substituted by 1, 2 or 3 substituents independently selected from halo and OH,
   or two R₃ substituents on the same ring carbon atom may join, together with the carbon atom to which they are attached, to form a cyclopropyl ring;
x is 0, 1 or 2;
R₄ is selected from:
—(C₁-C₄)alkyl;
-heteroaryl1, wherein said heteroaryl1 is a 5 or 6 membered, fully unsaturated, monocyclic ring comprising ring carbon atoms and 1, 2, 3 or 4 ring heteroatoms independently selected from N, O and S;
-heteroaryl2, wherein said heteroaryl2 is a 9 or 10 membered fused bicyclic ring comprising ring carbon atoms and 1, 2, 3 or 4 ring heteroatoms independently selected from N, O and S, and wherein both rings are fully unsaturated, or one ring is fully unsaturated, and the other is saturated or partially unsaturated, and wherein the heteroatoms may be in one or both rings;
-phenyl;
wherein heteroaryl1, heteroaryl2 and phenyl are each substituted by 1, 2 or 3 substituents independently selected from R₁₀, R₁₁, R₁₂, R₁₃ and R₁₄, wherein each R₁₀, R₁₁, R₁₂, R₁₃ and R₁₄ is independently selected from:
   H,
   halo,
   (C₁-C₄)alkyl unsubstituted or substituted by 1, 2 or 3 halo substituents,
   (C₁-C₂)alkyl substituted by —O—(C₁-C₂)alkyl or OH,
   —S—(C₁-C₃)alkyl,
   —O—(C₁-C₄)alkyl unsubstituted or substituted by 1, 2 or 3 halo substituents,
   OH,
   (C₃-C₅)cycloalkyl, wherein said (C₃-C₅)cycloalkyl is unsubstituted or substituted by 1 or 2 halo,
   —O—(C₃-C₅)cycloalkyl,
   —NR₃₄R₃₅ wherein R₃₄ and R₃₅ are independently selected from:
      H,
      (C₁-C₄)alkyl, wherein said (C₁-C₄)alkyl is unsubstituted or substituted by OH or —O(C₁-C₂)alkyl,
      and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
   CN,
   —(C₂-C₄)alkenyl,
   —(C₂-C₄)alkynyl,
   ═O
   —C(O)H, and
   —C(O)(C₁-C₄)alkyl;
and * indicates a point of attachment.

In another aspect, the invention provides a pharmaceutical composition comprising a compound of formula (I) of the present invention and one or more pharmaceutically acceptable carriers.

In another aspect, the invention provides a combination, in particular a pharmaceutical combination, comprising a compound of formula (I) of the present invention and one or more therapeutically active agents.

In another aspect, the invention provides a compound of formula (I) of the present invention for use as a medicament, in particular for the treatment of a disorder or disease which can be treated by WRN inhibition.

In another aspect, the invention provides a compound of formula (I) of the present invention for use in the treatment of cancer, particularly wherein the cancer is characterized as microsatellite instability-high (MSI-H) or mismatch repair deficient (dMMR).

In another aspect, the invention provides a method of treating a disorder or disease which can be treated by WRN inhibition in a subject, comprising administering to the subject a therapeutically effective amount of a compound of formula (I) of the present invention.

In another aspect, the invention provides a method of treating cancer in a subject, more particularly wherein the cancer is characterized as microsatellite instability-high (MSI-H) or mismatch repair deficient (dMMR), comprising administering to the subject a therapeutically effective amount of a compound of formula (I) of the present invention.

In another aspect, the invention provides the use of a compound of formula (I) of the present invention in the manufacture of a medicament for the treatment of a disorder or disease which can be treated by WRN inhibition.

In another aspect, the invention provides a compound of formula (I) of the present invention for use as a research chemical, for example as a chemical probe or as a tool compound.

In another aspect, the invention provides a solid form, process or intermediate as described herein.

### BRIEF DESCRIPTION OF THE DRAWINGS

FIG. 1 shows a X-ray powder diffractogram of the compound of Example 18A.

FIG. 2 shows efficacy of Example 18A against SW48 colorectal xenografts in Crl:NU(NCr)-Foxn1^{nu} mice FIG. 3 shows tolerability of Example 18A against SW48 colorectal xenografts in Crl:NU(NCr)-Foxn1^{nu} mice.

FIG. 4 shows efficacy of Example 21A against SW48 colorectal xenografts in Crl:NU(NCr)-Foxn1^{nu} mice.

FIG. 5 shows tolerability of Example 21A against SW48 colorectal xenografts in Crl:NU(NCr)-Foxn1^{nu} mice.

FIG. 6 shows a X-ray powder diffractogram of the compound of Example 21A.

### DETAILED DESCRIPTION

The invention therefore provides a compound of formula (I):

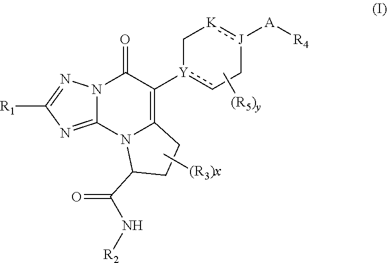

wherein R₁, R₂, R₃, x, Y, K, J R₄, R₅ and A are as described in the Summary of the Invention, supra.

Unless specified otherwise, the term “compounds of the present invention” or “compound of the present invention” or “a compound of formula (I)”, refers to a compound or compounds of formula (I), subformulae thereof, exemplified compounds, and salts thereof, as well as all zwitterions, stereoisomers (including diastereoisomers and enantiomers), rotamers, tautomers and isotopically labeled compounds (including deuterium substitutions), as well as inherently formed moieties, and combinations or mixtures of the above-mentioned aspects thereof.

Various (enumerated) embodiments of the invention are described herein. It will be recognized that features specified in each embodiment may be combined with other specified features to provide further embodiments of the present invention.

Embodiment 1. A compound of formula (I) or a pharmaceutically acceptable salt thereof, as described above.

Embodiment 2. A compound of formula (I) or a pharmaceutically acceptable salt thereof, according to Embodiment 1, wherein when R₁ is a ring, then:
each R₁ ring atom adjacent to the R₁ ring atom to which said R₁ ring is joined to the remainder of the molecule, is independently unsubstituted or substituted by halo only, in particular, independently unsubstituted or substituted with one F substituent, and
preferably, said R₁ ring is linked to the remainder of the molecule via a R₁ ring nitrogen atom, or a R₁ ring carbon atom which is double-bonded to an adjacent R₁ ring atom.

Embodiment 3. A compound of formula (I) or a pharmaceutically acceptable salt thereof, according to Embodiments 1 or 2, wherein R₁ is:
cycloalkenyl, wherein said cycloalkenyl is a partially unsaturated monocyclic ring containing 5 or 6 ring carbon atoms, and said cycloalkenyl is unsubstituted or substituted by 1, 2, 3 or 4, preferably 1 or 2, R₃₃, wherein R₃₃ is halo, and wherein said cycloalkenyl or halo-substituted cycloalkenyl is substituted by 0, 1 or 2 R₁₅ substituents,
or R₁ is heterocyclyl, wherein said heterocyclyl is a 5 or 6 membered fully saturated or partially unsaturated group comprising ring carbon atoms and 1 or 2 ring heteroatoms independently selected from N, NH, O and S, and wherein said heterocyclyl is unbridged or bridged, and said bridge is 1 or 2 carbon atoms, wherein said heterocyclyl is unsubstituted or substituted by 1, 2, 3 or 4, for example 1, 2 or 3, in particular 1 or 2 R₃₃, wherein R₃₃ is halo, and wherein said heterocyclyl or halo-substituted heterocyclyl is substituted by 0, 1 or 2 substituents independently selected from R₁₅, R₁₆, R₁₇, R₁₈, R₁₉, R₂₀, R₂₂ and R₂₃,
or R₁ is heteroaryl, wherein said heteroaryl is a 5 or 6 membered fully unsaturated monocyclic group comprising ring carbon atoms and 1, 2, 3 or 4 ring heteroatoms independently selected from N, O and S, preferably 1 or 2 ring heteroatoms, wherein the total number of ring S atoms does not exceed 1, and the total number of ring O atoms does not exceed 1, wherein said heteroaryl is unsubstituted or substituted by 1, 2 or 3 substituents independently selected from R₂₁ and R₃₀, wherein R₂₁ and R₃₀ are independently selected from halo and (C₁-C₄)alkyl, wherein said (C₁-C₄)alkyl is unsubstituted or substituted by 1, 2 or 3 halo, and each R₁₅, R₁₆, R₁₇, R₁₈, R₁₉, R₂₀, R₂₂ and R₂₃ is independently selected from:
   halo
   (C₁-C₄)alkyl-O— unsubstituted or substituted by 1, 2 or 3 halo;
   (C₁-C₄)alkyl unsubstituted or substituted by OH, —O—(C₁-C₂)alkyl or 1, 2 or 3 halo,
   HOC(O)—(CH₂)ₙ—,
   H₃C—C(O)(CH₂)ₙ—,
   (C₁-C₄)alkyl-O—C(O)(CH₂)ₙ,
   ═O
   azetidinyl or pyrrolidinyl, wherein said azetidinyl and pyrrolidinyl are linked to the rest of the molecule via the N atom, and are each unsubstituted or substituted by 1 or 2 F,
   R₂₅(R₂₄)N—, wherein R₂₄ is H or (C₁-C₄)alkyl unsubstituted or substituted by 1, 2 or 3 halo, R₂₅ is H or (C₁-C₄)alkyl unsubstituted or substituted by 1, 2 or 3 halo,
   OH
wherein n is 0, 1 or 2,

Embodiment 4. A compound of formula (I) or a pharmaceutically acceptable salt thereof, according to any of Embodiments 1, 2 or 3, wherein R₁ is:
cycloalkenyl, wherein said cycloalkenyl is a partially unsaturated monocyclic ring containing 5 or 6 ring carbon atoms, and said cycloalkenyl is unsubstituted or substituted by 1 or 2 R₃₃, wherein R₃₃ is halo, preferably F, and wherein said cycloalkenyl or halo-substituted cycloalkenyl is substituted by 0 or 1 R₁₅ substituents, preferably 1 substituent, wherein R₁₅ is selected from:
   a) (C₁-C₂)alkyl-O— unsubstituted or substituted by 1, 2 or 3 halo;
   b) (C₁-C₂)alkyl unsubstituted or substituted by 1, 2 or 3 halo,
   c) HOC(O)—(CH₂)ₙ—,
   d) H₃C—C(O)(CH₂)ₙ—,
   e) H₃C—O—C(O)(CH₂)ₙ,
   f) ═O, and
   g) R₂₅(R₂₄)N—, H, wherein R₂₄ is H or (C₁-C₂)alkyl unsubstituted or substituted by 1, 2 or 3 halo, R₂₅ is H or (C₁-C₂)alkyl unsubstituted or substituted by 1, 2 or 3 halo,
n is 0 or 1,
wherein
   the R₁₅ substituent a) to g) of said cycloalkenyl or halo-substituted cycloalkenyl is not present on the ring atoms adjacent to the ring atom to which the cycloalkenyl or halo-substituted cycloalkenyl is joined to the remainder of the molecule, and preferably, said cycloalkenyl or halo-substituted cycloalkenyl is a 6 membered ring, with 1 R₁₅ substituent in the ring para position relative to the remainder of the molecule; and
   said cycloalkenyl or halo-substituted cycloalkenyl is linked to the remainder of the compound via a R₁ ring carbon atom which is double bonded to an adjacent R₁ ring carbon atom;
or R₁ is heterocyclyl, wherein said heterocyclyl is a 5 or 6 membered fully saturated or partially unsaturated group comprising ring carbon atoms and 1 or 2 ring heteroatoms independently selected from N, NH, O and S, and wherein said heterocyclyl is unbridged or bridged, and said bridge is 1 or 2 carbon atoms, wherein said heterocyclyl is unsubstituted or substituted by 1 or 2 R₃₃, wherein R₃₃ halo, is preferably F, and wherein said heterocyclyl or halo-substituted heterocyclyl is substituted by 0 or 1 substituents independently selected from R₁₅, R₁₆, R₁₇, R₁₈, R₁₉, R₂₀, R₂₂ and R₂₃, wherein said R₁₅, R₁₆, R₁₇, R₁₈, R₁₉, R₂₀, R₂₂ and R₂₃ are independently selected from:
   a) (C₁-C₄)alkyl-O— unsubstituted or substituted by 1, 2 or 3 halo;
   b) (C₁-C₄)alkyl unsubstituted or substituted by OH, —O—(C₁-C₂)alkyl or 1, 2 or 3 halo,
   c) HOC(O)—(CH₂)ₙ—,
   d) H₃C—C(O)(CH₂)ₙ—,
   e) H₃C—O—C(O)(CH₂)ₙ,
   f)═O
   g) R₂₅(R₂₄)N—, wherein R₂₄ is H, (C₁-C₂)alkyl unsubstituted or substituted by 1, 2 or 3 halo, R₂₅ is H, (C₁-C₂)alkyl unsubstituted or substituted by 1, 2 or 3 halo,
   h) OH
wherein n is 0 or 1,
and wherein:
   substituent a) to h) of said heterocyclyl or halo-substituted heterocyclyl is not present on the ring atoms adjacent to the ring atom to which the heterocyclyl or halo-substituted heterocyclyl is joined to the remainder of the molecule, and preferably, when said heterocyclyl or halo-substituted heterocyclyl is a 6 membered ring, it has 0 or 1 substituent selected from a) to h) in the meta or para position, preferably para, relative to the remainder of the molecule; and
   said heterocyclyl is linked to the remainder of the compound via a R₁ ring nitrogen atom, or a R₁ ring carbon atom which is double bonded to an adjacent ring atom;
or R₁ is heteroaryl, wherein said heteroaryl is a 5 or 6 membered fully unsaturated monocyclic group comprising ring carbon atoms and 1 or 2 ring heteroatoms independently selected from N, O and S, preferably N, wherein the total number of ring S atoms does not exceed 1, and the total number of ring O atoms does not exceed 1, wherein said heteroaryl is unsubstituted or substituted by 1 or 2 substituents independently selected from R₂₁ and R₃₀, wherein R₂₁ and R₃₀ are independently selected from (C₁-C₂)alkyl, and said (C₁-C₂)alkyl is unsubstituted or substituted by 1, 2 or 3 halo, and wherein preferably, said alkyl or halo-alkyl substituent is not present on the R₁ ring atoms adjacent to the R₁ ring atom to which the heteroaryl is joined to the remainder of the molecule, and more preferably, when heteroaryl is a 6-membered ring, said alkyl or halo-alkyl substituent is in the ring para position relative to the rest of the molecule.

Embodiment 5. A compound of formula (I) or a pharmaceutically acceptable salt thereof, according to any of Embodiments 1 to 4, wherein R₁ is selected from:

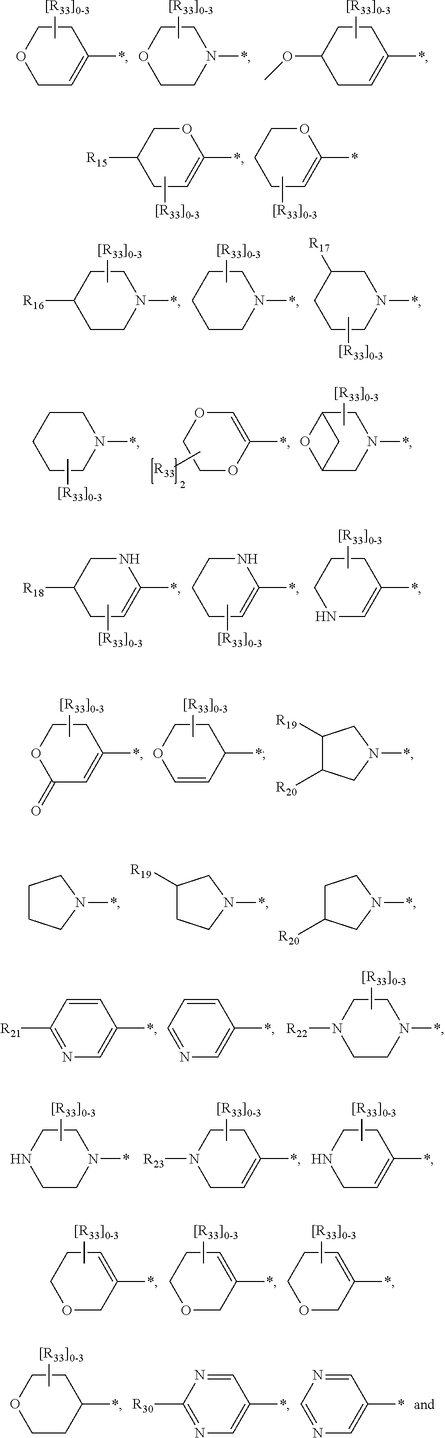

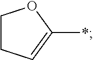

alternatively, there are 0-2 R₃₃ substituents, in each of the moieties above,
R₃₃ is F;
R₁₅ is halo, azetidinyl or pyrrolidinyl, wherein said azetidinyl and pyrrolidinyl are linked to the rest of the molecule via the N atom, and are unsubstituted or substituted by 1 or 2 F;
R₁₆ is R₂₅(R₂₄)N—, wherein R₂₄ is H or (C₁-C₂)alkyl, R₂₅ is H or (C₁-C₂)alkyl unsubstituted or substituted by 1, 2 or 3 halo, in particular F;
R₁₇ is halo
R₁₈ is halo;
R₁₉ is halo;
R₂₀ is halo;
R₂₁ is (C₁-C₂)alkyl;
R₂₂ and R₂₃ are each independently selected from:
   (C₁-C₄)alkyl unsubstituted or substituted by 1, 2 or 3 halo,
   HOC(O)—(CH₂)ₙ—,
   H₃C—C(O)(CH₂)ₙ—,
   (H₃C)₃C—O—C(O)(CH₂)ₙ—;
   wherein n is 0, 1 or 2;
and
R₃₀ is CH₃.

Embodiment 6. A compound of formula (I) or a pharmaceutically acceptable salt thereof, according to any of Embodiments 1 to 5, wherein R₁ is selected from:

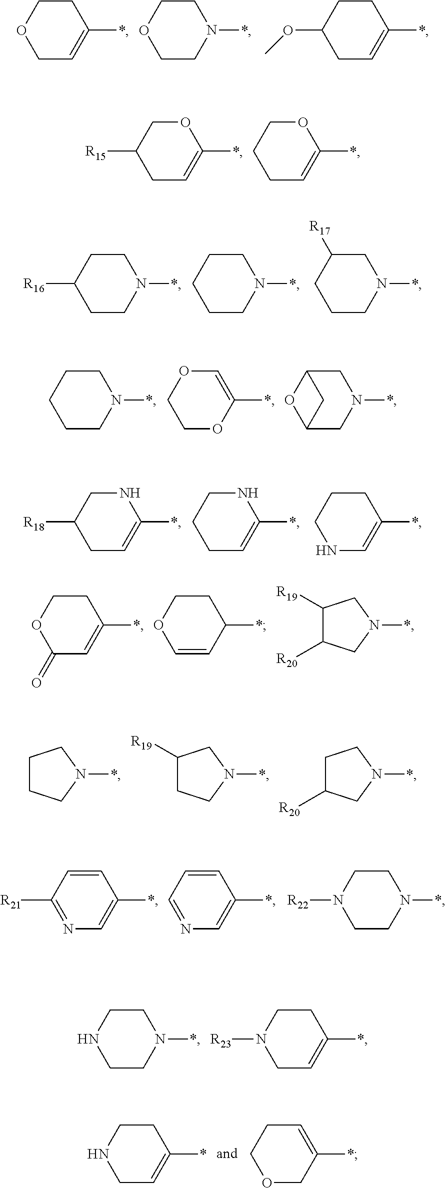

R₁₅ is F;
R₁₆ is R₂₅(R₂₄)N—;
R₁₇ is F;
R₁₈ is F;
R₁₉ is F;
R₂₀ is F;
R₂₁ is CH₃;
R₂₂ is CF₃, CHF₂CH₂, HOC(O)—CH₂—, H₃C—C(O)—, (H₃C)₃C—O—C(O)—;
R₂₃ is CF₃, CHF₂CH₂—, (H₃C)₃C—O—C(O)—;
R₂₄ is CH₃; and
R₂₅ is CHF₂CH₂—.

Embodiment 7. A compound of formula (I), or a pharmaceutically acceptable salt thereof, according to any of Embodiments 1 to 6, wherein R₁ is selected from:

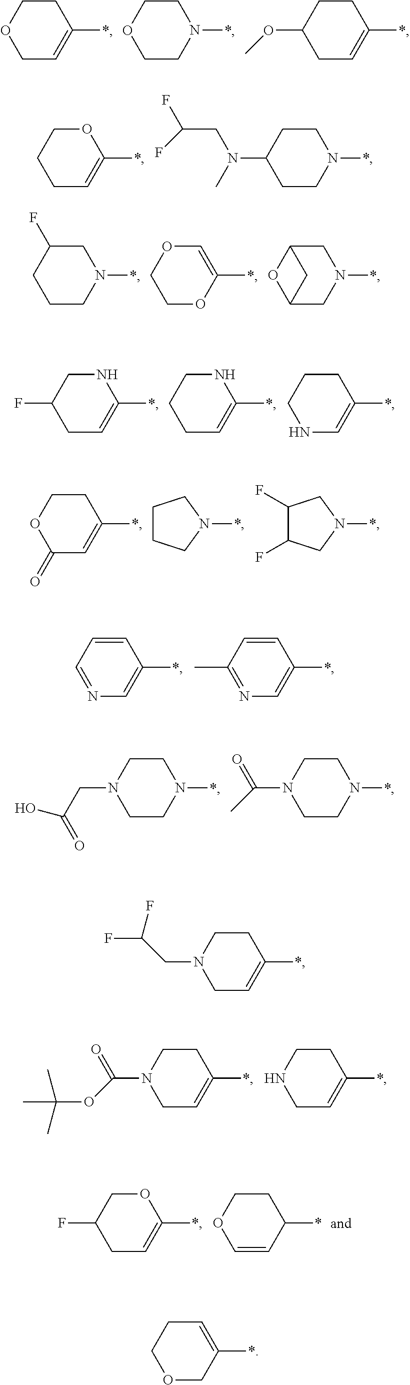

Embodiment 8. A compound of formula (I), or a pharmaceutically acceptable salt thereof, according to embodiment 7, wherein R₁ is selected from:

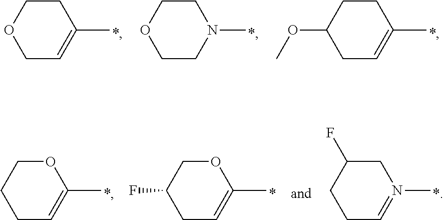

Embodiment 9. A compound of formula (I), or a pharmaceutically acceptable salt thereof, according to embodiment 8, wherein R₁ is selected from:

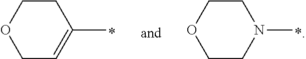

Embodiment 10. A compound of formula (I), or a pharmaceutically acceptable salt thereof, according to embodiment 9, wherein R₁ is:

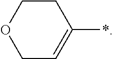

Embodiment 11. A compound of formula (I) or a pharmaceutically acceptable salt thereof, according to any of Embodiments 1 to 10, wherein R₂ is the moiety:

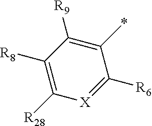

R₆ is selected from:
   H,
   halo,
   (C₁-C₄)alkyl unsubstituted or substituted by 1, 2 or 3 halo,
   (C₃-C₅)cycloalkyl unsubstituted or substituted by 1, 2 or 3 halo,
   —O—(C₁-C₄)alkyl unsubstituted or substituted by 1, 2 or 3 halo,
   OH, and
   CN;
R₈ is selected from H, halo, and (C₁-C₄)alkyl unsubstituted or substituted by 1, 2 or 3 halo,
R₉ is selected from H, O—CH₃, OH, CN, CH₃ and halo;
R₂₈ is selected from:
   SF₅,
   H,
   —C(O)H,
   halo,
   (C₁-C₄)alkyl unsubstituted or substituted by 1, 2 or 3 halo,
   (C₁-C₄)alkynyl,
   (C₁-C₄)alkenyl,
   (C₃-C₅)cycloalkyl unsubstituted or substituted by 1, 2 or 3 halo, and
   OCF₃;
and X is selected from C—R₇ and N, wherein R₇ is H or halo.

Embodiment 12. A compound of formula (I) or a pharmaceutically acceptable salt thereof, according to any of Embodiments 1 to 11, wherein R₂ is the moiety:

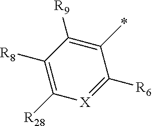

wherein
R₆ is selected from H, halo, (C₁-C₄)alkyl unsubstituted or substituted by 1, 2 or 3 halo;
R₈ is selected from H, halo, (C₁-C₄)alkyl unsubstituted or substituted by 1, 2 or 3 halo;
R₉ is selected from H, O—CH₃, OH, CN, CH₃ and halo;
R₂₈ is selected from SF₅, halo, C(O)H and (C₁-C₄)alkyl unsubstituted or substituted by 1, 2 or 3 halo;
X is selected from C—R₇ and N; and
R₇ is selected from H and halo.

Embodiment 13. A compound of formula (I) or a pharmaceutically acceptable salt thereof, according to any of Embodiments 1 to 12, wherein R₂ is the moiety:

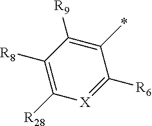

R₆ is selected from H, Cl, CH₃, F and Br;
R₈ is selected from H, Cl, F and CF₃;
R₉ is selected from H, CH₃ and Cl;
R₂₈ is selected from CF₃, CF₂H, —CH₂CH₃, Cl, SF₅, Br and —C(O)H;
X is selected from C—R₇ and N; and
R₇ is selected from H and F.

Embodiment 14. A compound of formula (I), or a pharmaceutically acceptable salt thereof, according to any of embodiments 1-13, wherein the moiety:

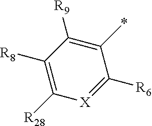

is selected from:

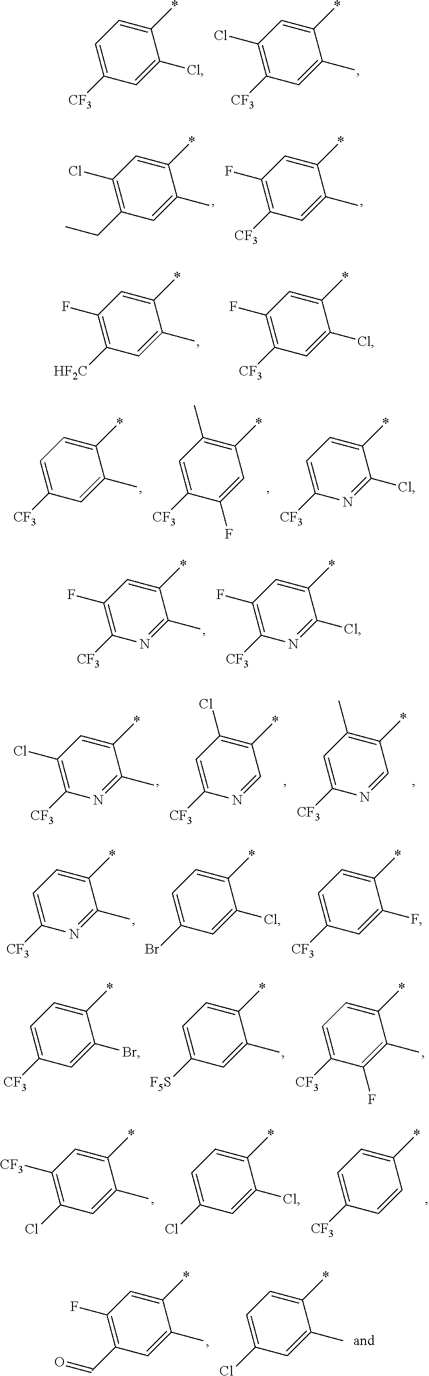

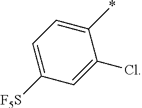

Embodiment 15. A compound of formula (I), or a pharmaceutically acceptable salt thereof, according to embodiment 14, wherein the moiety:

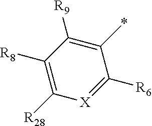

is selected from:

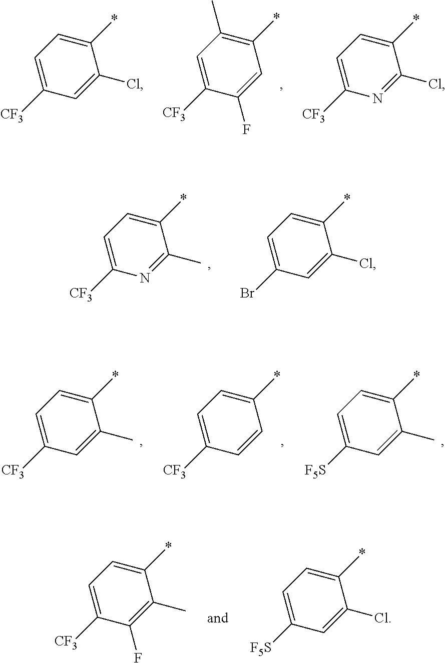

Embodiment 16. A compound of formula (I), or a pharmaceutically acceptable salt thereof, according to embodiment 15, wherein the moiety:

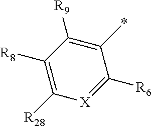

is selected from:

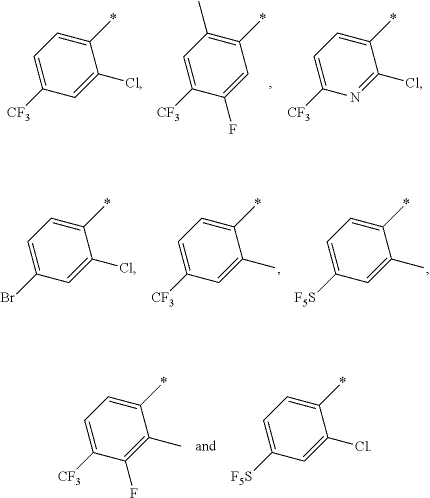

Embodiment 17. A compound of formula (I), or a pharmaceutically acceptable salt thereof, according to embodiment 16, wherein the moiety:

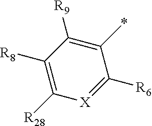

is selected from:

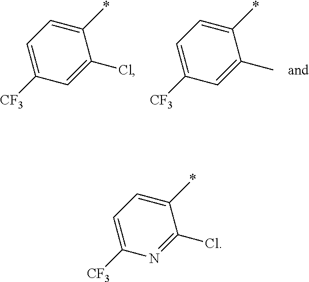 [SMILES: *c1ccc(C(F)(F)F)cc1Cl ; *c1ccc(C(F)(F)F)cc1C ; "and" ; *c1ccc(C(F)(F)F)nc1Cl]

Embodiment 18. A compound of formula (I) or a pharmaceutically acceptable salt thereof, according to any of Embodiments 1 to 17, wherein x is 0 or 1. In particular x is 1.

Embodiment 19. A compound of formula (I) or a pharmaceutically acceptable salt thereof, according to any of Embodiments 1 to 18, wherein R₃ is (C₁-C₄)alkyl unsubstituted or substituted by 1, 2 or 3 substituents independently selected from halo and OH.

Embodiment 20. A compound of formula (I) or a pharmaceutically acceptable salt thereof, according to any of Embodiments 1 to 19, wherein R₃ is (C₁-C₂)alkyl unsubstituted or substituted by 1, 2 or 3 substituents independently selected from halo and OH, preferably R₃ is —CH₂CH₃ or CH₃, more preferably CH₃.

Embodiment 21. A compound of formula (I) or a pharmaceutically acceptable salt thereof, according to any of Embodiments 1 to 20, wherein R₃ is CH₃.

Embodiment 22. A compound of formula (I) or a pharmaceutically acceptable salt thereof, according to any of Embodiments 1 to 21, wherein R₃ is in the position shown in formula 1i:

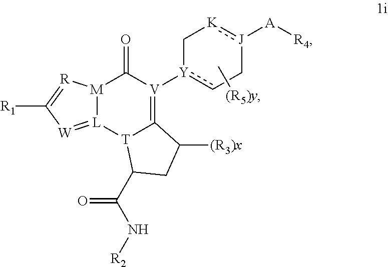

Embodiment 23. A compound of formula (I) or a pharmaceutically acceptable salt thereof, according to any of Embodiments 1 to 22, wherein Y is N and Y [symbol] is Y linked by a single bond.

Embodiment 24. A compound of formula (I) or a pharmaceutically acceptable salt thereof, according to any of Embodiments 1 to 23, wherein Y [symbol] is K linked by a single bond, and K is selected from —CH₂—, —CH₂CH₂—, —NH— and a bond (to form a 5-membered ring:

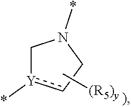

J is N, and A is a linker selected from —C(O)—, —S(O)—, —S(O)₂—, and

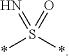

Embodiment 25. A compound of formula (I) or a pharmaceutically acceptable salt thereof, according to any of Embodiments 1 to 24, wherein K [symbol] is K linked by a single bond, K is —CH₂—, J is N, and A is a linker selected from —C(O)—, —S(O)—, —S(O)₂—, and

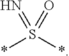

Embodiment 26. A compound of formula (I) or a pharmaceutically acceptable salt thereof, according to any of Embodiments 1 to 25, wherein A is a linker selected from —C(O)— and —S(O)₂—, preferably —C(O)—.

Embodiment 27. A compound of formula (I) or a pharmaceutically acceptable salt thereof, according to any of Embodiments 1 to 26, wherein R₅ is independently selected from:
(C₁-C₄)alkyl, preferably methyl,
and wherein two R₅ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S,
when K [symbol] J is a carbon-nitrogen single bond, a R₅ substituent on K and on the adjacent carbon atom may join to form ring C:

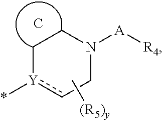

wherein ring C is a fused (C₃-C₆)cycloalkyl ring, in particular a fused cyclobutyl ring, a fused (C₃-C₆)heterocyclyl ring or a fused phenyl ring, wherein said fused (C₃-C₆)heterocyclyl ring contains ring carbon atoms and one ring heteroatom selected from O, N and S,
   and wherein when ring C is a fused (C₃-C₆)cycloalkyl ring, in particular fused cyclobutyl ring, said fused (C₃-C₆)cycloalkyl ring is unsubstituted or substituted with 1 or 2 R₄₀ groups, wherein said R₄₀ is selected from:
      (C₁-C₂)alkyl, wherein each (C₁-C₂)alkyl is independently unsubstituted or substituted by OH or 1, 2 or 3 halo,
      halo, in particular F,
      or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
      or wherein two R₄₀ substituents on adjacent carbon atoms join together with the carbon atoms to which they are attached, to form a fused cyclopropyl ring;
and wherein when K is —CH₂— and J is N, two R₅ substituents may join to form a (C₁-C₃)alkylene bridge or a heteroalkylene bridge, wherein said heteroalkylene bridge is one heteroatom selected from N and O, or is —CH₂—O—CH₂—.

Embodiment 28. A compound of formula (I) or a pharmaceutically acceptable salt thereof, according to any of Embodiments 1 to 27, wherein R₅ is independently selected from:
(C₁-C₄)alkyl, preferably methyl,
when K [symbol] J is a carbon-nitrogen single bond, a R₅ substituent on K and on the adjacent carbon atom may join to form ring C:

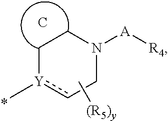

wherein ring C is a fused (C₃-C₆)cycloalkyl ring, in particular a fused cyclobutyl ring, or a fused (C₃-C₆)heterocyclyl ring, wherein said fused (C₃-C₆)heterocyclyl ring contains ring carbon atoms and one ring heteroatom selected from O, N and S,
   and wherein when ring C is a fused (C₃-C₆)cycloalkyl ring, in particular fused cyclobutyl ring, said fused (C₃-C₆)cycloalkyl ring is unsubstituted or substituted with 1 or 2 R₄₀ groups, wherein said R₄₀ is selected from:
      (C₁-C₂)alkyl, wherein each (C₁-C₂)alkyl is independently unsubstituted or substituted by OH or 1, 2 or 3 halo,
      halo, in particular F,
      or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
      or wherein two R₄₀ substituents on adjacent carbon atoms join together with the carbon atoms to which they are attached, to form a fused cyclopropyl ring;
and wherein when K is —CH₂— and J is N, two R₅ substituents may join to form a (C₁-C₃)alkylene bridge or a heteroalkylene bridge, wherein said heteroalkylene bridge is one heteroatom selected from N and O, or is —CH₂—O—CH₂—.

Embodiment 29. A compound of formula (I) or a pharmaceutically acceptable salt thereof, according to any of Embodiments 1 to 28, wherein R₅ is independently selected from:
—(C₁-C₂)alkyl, preferably methyl, and
when K [symbol] J is a carbon-nitrogen single bond, a R₅ substituent on K and on the adjacent carbon atom may join to form ring C:

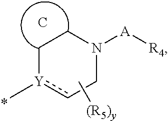

wherein ring C is a fused (C₃-C₄)cycloalkyl ring, in particular a fused cyclobutyl ring, and said fused (C₃-C₄)cycloalkyl ring, in particular fused cyclobutyl ring, is unsubstituted or substituted with 1 or 2 R₄₀ groups as described in embodiment 28.

Embodiment 30. A compound of formula (I) or a pharmaceutically acceptable salt thereof, according to any of Embodiments 1 to 29, wherein y is 0, 1, 2 or 3, preferably 0, 1, or 2.

Embodiment 31. A compound of formula (I) or a pharmaceutically acceptable salt thereof, according to any of Embodiments 1 to 30, wherein R₅ is independently selected from:
CH₃, and y is 1 or 2, and
when K [symbol] J is a carbon-nitrogen single bond, a R₅ substituent on K and on the adjacent carbon atom may join to form ring C:

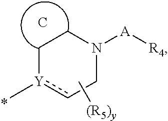

wherein ring C is a fused cyclobutyl ring.

Embodiment 32. A compound of formula (I) or a pharmaceutically acceptable salt thereof, according to any of Embodiments 1 to 31, wherein the compound of formula (I) includes the moiety:

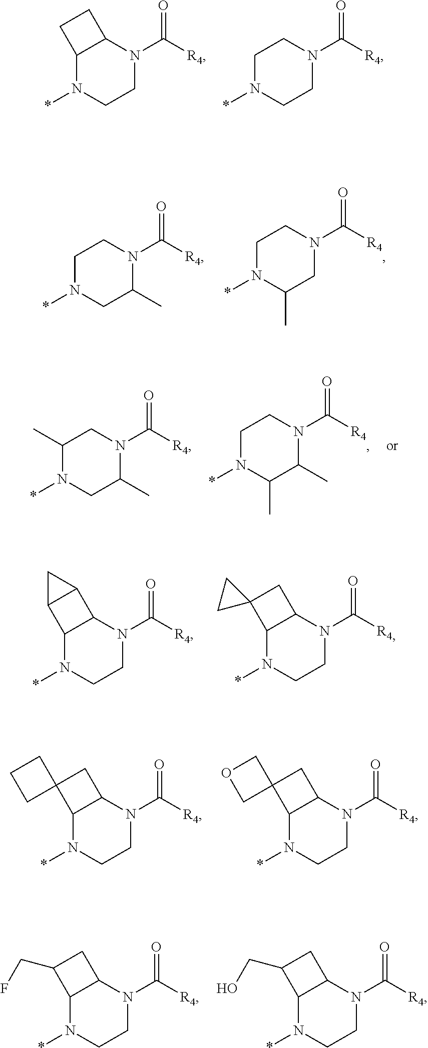

in particular
A:

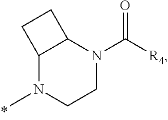

more particularly

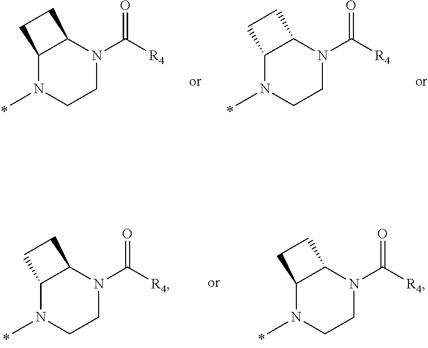

or B:

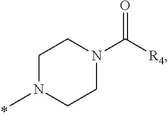

or C:

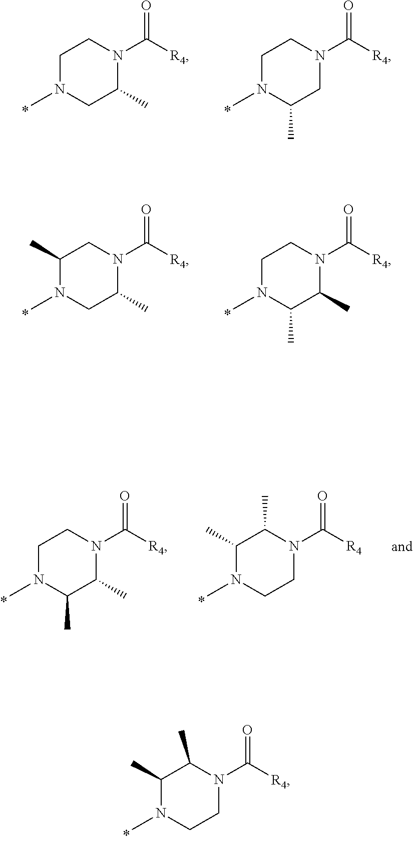

or wherein the linker-C(O)— is replaced by the alternative linkers —S(O)—, —S(O)₂—, and

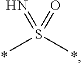

as defined in claim 1.

Embodiment 33. A compound of formula (I) or a pharmaceutically acceptable salt thereof, according to any of Embodiments 1 to 32, wherein R₄ is selected from:
—(C₁-C₄)alkyl;
-heteroaryl1, wherein said heteroaryl1 is a 5 or 6 membered, fully unsaturated monocyclic ring comprising ring carbon atoms and 1, 2, 3 or 4 ring heteroatoms independently selected from N, O and S, and the total number of ring S atoms does not exceed 1, and the total number of ring O atoms does not exceed 1;
-heteroaryl2, wherein said heteroaryl2 is a 9 or 10 membered fused bicyclic ring comprising ring carbon atoms and 1, 2, 3 or 4 ring heteroatoms independently selected from N, O and S, and wherein both rings are fully unsaturated, or one ring is fully unsaturated and the other is saturated or partially unsaturated, and wherein the heteroatoms may be in one or both rings, and the total number of ring S atoms does not exceed 1, and the total number of ring O atoms does not exceed 1, and in particular, the ring which is linked to the rest of the molecule via linker -A- is fully unsaturated;
-phenyl;
wherein heteroaryl1, heteroaryl2 and phenyl are each substituted by 1, 2 or 3 substituents independently selected from R₁₀, R₁₁, R₁₂, R₁₃ and R₁₄, wherein each R₁₀, R₁₁, R₁₂, R₁₃ and R₁₄ is independently selected from:
   H,
   halo,
   (C₁-C₄)alkyl unsubstituted or substituted by 1, 2 or 3 halo substituents,
   (C₁-C₂)alkyl substituted by —O—(C₁-C₂)alkyl or OH,
   —S—(C₁-C₃)alkyl,
   —O—(C₁-C₄)alkyl unsubstituted or substituted by 1, 2 or 3 halo substituents,
   OH,
   (C₃-C₅)cycloalkyl, wherein said (C₃-C₅)cycloalkyl is unsubstituted or substituted by 1 or 2 halo,
   —O—(C₃-C₅)cycloalkyl,
   —NR₃₄R₃₅ wherein R₃₄ and R₃₅ are independently selected from:
   H,
      (C₁-C₄)alkyl, wherein said (C₁-C₄)alkyl is unsubstituted or substituted by OH or —O(C₁-C₂)alkyl,
      and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
   CN,
   —(C₂-C₄)alkenyl,
   —(C₂-C₄)alkynyl,
   ═O
   —C(O)H, and
   —C(O)(C₁-C₄)alkyl;
with the proviso that one OH substituent is present on heteroaryl1, heteroaryl2 and phenyl, and the remaining R₁₀, R₁₁, R₁₂, R₁₃ and R₁₄ are as defined herein.

Embodiment 34. A compound of formula (I) or a pharmaceutically acceptable salt thereof, according to any of Embodiments 1 to 33, wherein R₄ is selected from:
—(C₁-C₄)alkyl, in particular —CH₃;
-heteroaryl1; and
-heteroaryl2;
-phenyl;
wherein heteroaryl1, heteroaryl2 and phenyl are each substituted by 1, 2 or 3 substituents independently selected from R₁₀, R₁₁, R₁₂, R₁₃ and R₁₄, wherein each R₁₀, R₁₁, R₁₂, R₁₃ and R₁₄ is independently selected from:
   H,
   halo,
   (C₁-C₄)alkyl unsubstituted or substituted by 1, 2 or 3 halo substituents,
   (C₁-C₂)alkyl substituted by —O—(C₁-C₂)alkyl or OH,
   —S—(C₁-C₃)alkyl,
   —O—(C₁-C₄)alkyl unsubstituted or substituted by 1, 2 or 3 halo substituents,
   OH,
   (C₃-C₅)cycloalkyl, wherein said (C₃-C₅)cycloalkyl is unsubstituted or substituted by 1 or 2 halo,
   —O—(C₃-C₅)cycloalkyl,
   —NR₃₄R₃₅ wherein R₃₄ and R₃₅ are independently selected from:
      H,
      (C₁-C₄)alkyl, wherein said (C₁-C₄)alkyl is unsubstituted or substituted by OH or —O(C₁-C₂)alkyl,
      and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
   CN,
   —(C₂-C₄)alkenyl,
   —(C₂-C₄)alkynyl,
   ═O
   —C(O)H, and
   —C(O)(C₁-C₄)alkyl;
with the proviso that:
   one OH substituent is present on heteroaryl1, heteroaryl2 and phenyl, and said OH is in the ortho position of the R₄ ring, relative to the position linking R₄ to linker -A-,
or
   one ═O substituent is present on heteroaryl1 and heteroaryl2,
and the remaining R₁₀, R₁₁, R₁₂, R₁₃ and R₁₄ are defined as herein.

Embodiment 35. A compound of formula (I) or a pharmaceutically acceptable salt thereof, according to any of Embodiments 1 to 34, wherein R₄ is selected from:
—(C₁-C₄)alkyl, in particular —CH₃;
-heteroaryl1; and
-heteroaryl2;
-phenyl;
wherein heteroaryl1, heteroaryl2 and phenyl are each substituted by 1, 2 or 3 substituents independently selected from R₁₀, R₁₁, R₁₂, R₁₃ and R₁₄, wherein each R₁₀, R₁₁, R₁₂, R₁₃ and R₁₄ is independently selected from: OH, ═O, H, F, Cl and CH₃, with the proviso that:
   one OH substituent is present on heteroaryl1, heteroaryl2 and phenyl, and said OH is in the ortho position of the R₄ ring, relative to the position linking R₄ to linker -A-, and the remaining substituents are selected from H, F, Cl, and CH₃,
or
   one ═O substituent on said is present on heteroaryl1 and heteroaryl2, and the remaining substituents are selected from H, F, Cl, and CH₃.

Embodiment 36. A compound of formula (I) or a pharmaceutically acceptable salt thereof, according to any of Embodiments 1 to 35, wherein R₄ is selected from CH₃, heteroaryl1 and heteroaryl2, and the substituents are as defined above. In particular, said heteraryl1 comprises ring carbon atoms and one or two nitrogen atoms only. More particularly, heteroaryl1 is pyridyl or pyrimidinyl.

Embodiment 37. A compound of formula (I) or a pharmaceutically acceptable salt thereof, according to any of Embodiments 1 to 36, wherein R₄ is selected from: —(C₁-C₄)alkyl, in particular CH₃;

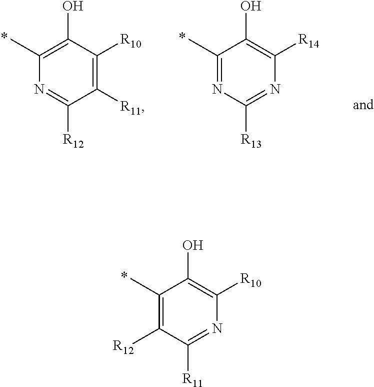

wherein
R₁₀, R₁₁, R₁₂, R₁₃ and R₁₄ are independently selected from:
   H,
   halo,
   (C₁-C₄)alkyl unsubstituted or substituted by 1, 2 or 3 halo substituents,
   (C₁-C₂)alkyl substituted by —O—(C₁-C₂)alkyl or OH,
   —S—(C₁-C₃)alkyl,
   —O—(C₁-C₄)alkyl unsubstituted or substituted by 1, 2 or 3 halo substituents,
   (C₃-C₅)cycloalkyl, wherein said (C₃-C₅)cycloalkyl is unsubstituted or substituted by 1 or 2 halo,
   —O—(C₃-C₅)cycloalkyl,
   —NR₃₄R₃₅ wherein R₃₄ and R₃₅ are independently selected from:
      H,
      (C₁-C₄)alkyl, wherein said (C₁-C₄)alkyl is unsubstituted or substituted by OH or —O(C₁-C₂)alkyl,
      and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
   CN,
   —(C₂-C₄)alkenyl,
   —(C₂-C₄)alkynyl,
   —C(O)H, and
   —C(O)(C₁-C₄)alkyl.

Embodiment 38. A compound of formula (I) or a pharmaceutically acceptable salt thereof, according to any of Embodiments 1 to 37, wherein R₄ is selected from:

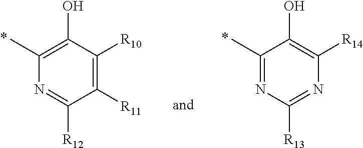

wherein
R₁₀ is selected from H, halo, (C₁-C₂)alkyl unsubstituted or substituted by 1, 2 or 3 halo substituents, —O—(C₁-C₂)alkyl unsubstituted or substituted by 1, 2 or 3 halo substituents;
R₁₁ is selected from H, halo, (C₁-C₂)alkyl unsubstituted or substituted by 1, 2 or 3 halo substituents;
R₁₂ is selected from H, halo, (C₁-C₂)alkyl unsubstituted or substituted by 1, 2 or 3 halo substituents;
R₁₃ is selected from H, —S—CH₃, halo, (C₁-C₂)alkyl unsubstituted or substituted by 1, 2 or 3 halo substituents; and
R₁₄ is selected from H, halo, (C₁-C₂)alkyl unsubstituted or substituted by 1, 2 or 3 halo substituents, O—(C₁-C₂)alkyl unsubstituted or substituted by 1, 2 or 3 halo substituents, and cyclopropyl.

Embodiment 39. A compound of formula (I) or a pharmaceutically acceptable salt thereof, according to any of Embodiments 1 to 38, wherein R₄ is selected from:

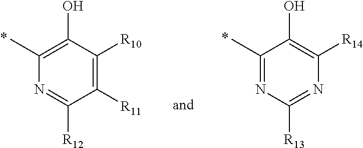

wherein
R₁₀ is selected from H, F, Cl, CH₃ and OCF₃;
R₁₁ is selected from H, F, Cl and CH₃;
R₁₂ is selected from H, F, Cl and CH₃;
R₁₃ is selected from H, F, Cl, —S—CH₃ and CH₃; and
R₁₄ is selected from H, F, Cl, CH₃, —CH₂CH₃, cyclopropyl, —OCHF₂, OCF₃.

In particular, at least one substituent of R₁₀, R₁₁ and R₁₂, is H.

In particular, at least one substituent of R₁₃ and R₁₄, is H.

Embodiment 40. A compound of formula (I), or a pharmaceutically acceptable salt thereof, according to any of embodiments 1 to 39, wherein R₄ is selected from:
—CH₃,

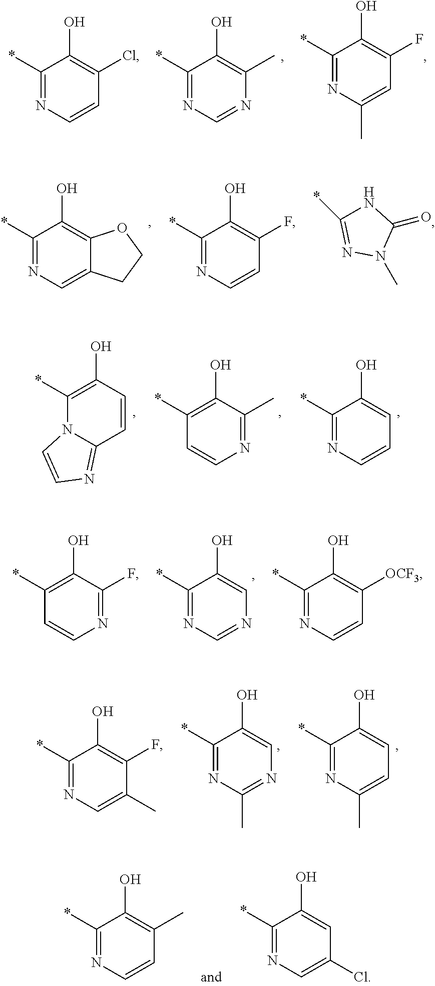

Embodiment 41. A compound of formula (I), or a pharmaceutically acceptable salt thereof, according to any of embodiments 1 to 40, wherein R₄ is selected from:
—CH₃,

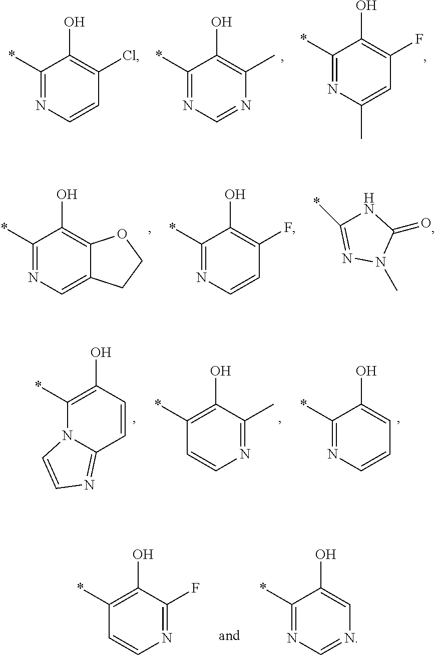

Embodiment 42. A compound of formula (I), or a pharmaceutically acceptable salt thereof, according to any of Embodiments 1 to 41, wherein R₄ is selected from:

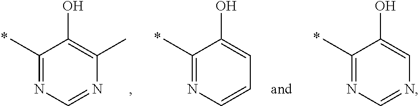

in particular

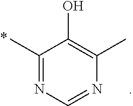

Embodiment 43. A compound of formula (I), or a pharmaceutically acceptable salt thereof, according to any of Embodiments 1 to 42, wherein the compound of formula (I) has the stereochemistry shown in formula (I′):

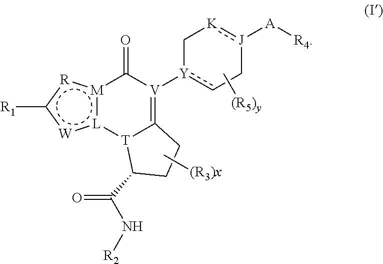

In particular, the compound of formula (I) is a compound of formula (I″):

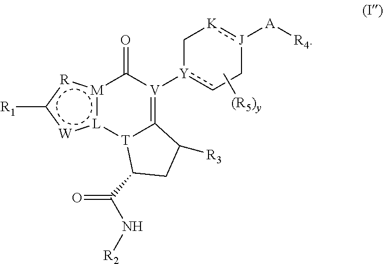

More particularly, said compound has the stereochemistry of formula (I′″):

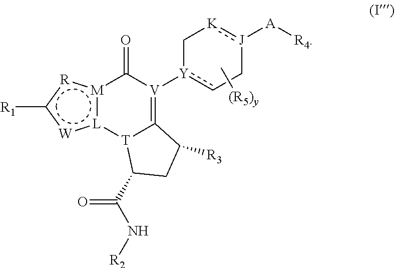

Embodiment 44. A compound of formula (I), or a pharmaceutically acceptable salt thereof, according to any of Embodiments 1 to 43, wherein formula (I) is formula 1a:

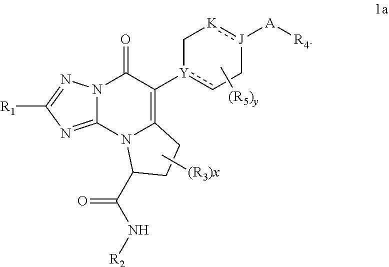

(Preferably, formula (I) is formula 1a).

In a particular embodiment, there is provided a compound of formula 1a′:

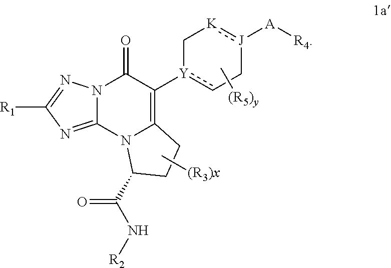

Embodiment 45. A compound of formula (I) or a pharmaceutically acceptable salt thereof, according to any of Embodiments 1 to 43, wherein formula (I) is formula 1b:

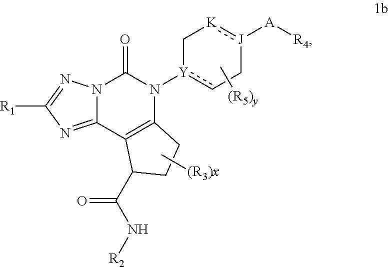

or 1b′:

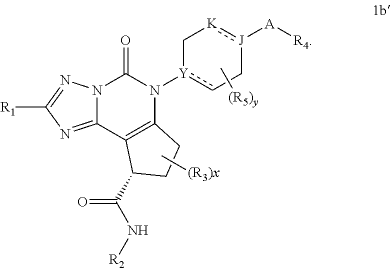

Embodiment 46. A compound of formula (I) or a pharmaceutically acceptable salt thereof, according to any of Embodiments 1 to 43, wherein formula (I) is formula 1c:

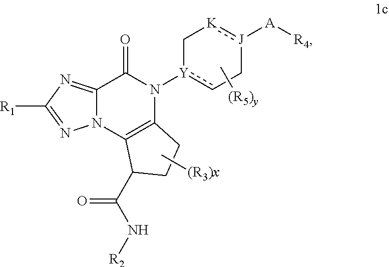

or 1c′:

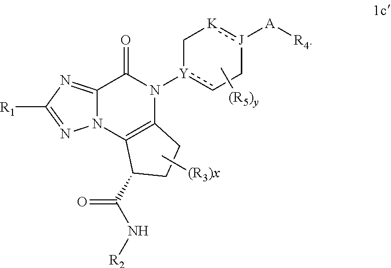

Embodiment 47. A compound of formula (I) or a pharmaceutically acceptable salt thereof, according to any of Embodiments 1 to 43, wherein formula (I) is formula 1d:

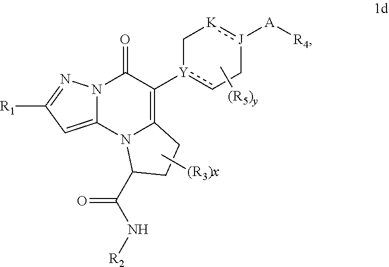

or 1d′:

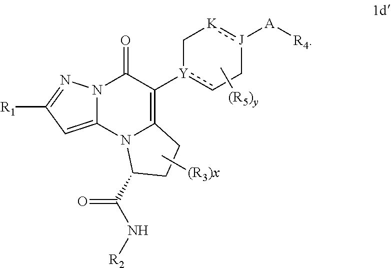

Embodiment 48. A compound of formula (I) or a pharmaceutically acceptable salt thereof, according to any of Embodiments 1 to 43, wherein formula (I) is formula 1e:

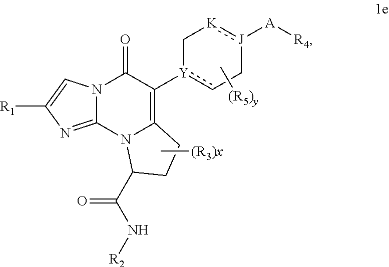

or 1e′:

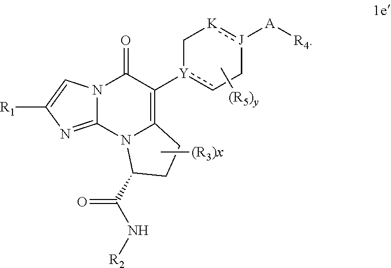

Embodiment 49. A compound of formula (I) or a pharmaceutically acceptable salt thereof, according to any of Embodiments 1 to 43, wherein formula (I) is formula 1f:

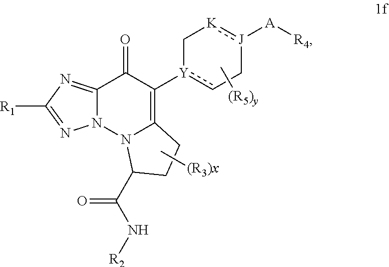

or 1f:

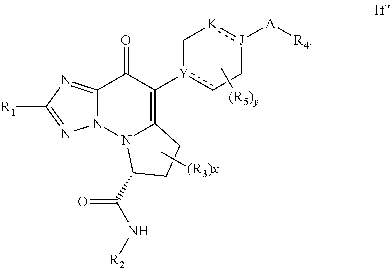

Embodiment 50. A compound of formula (I), or a pharmaceutically acceptable salt thereof, according to any of Embodiments 1 to 44, wherein formula (I) is formula 1g:

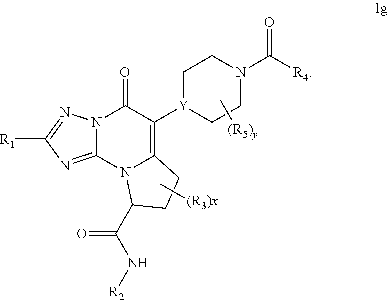

More preferably, formula (I) is formula 1g.

There is also provided a compound of formula (I) which is formula 1g′:

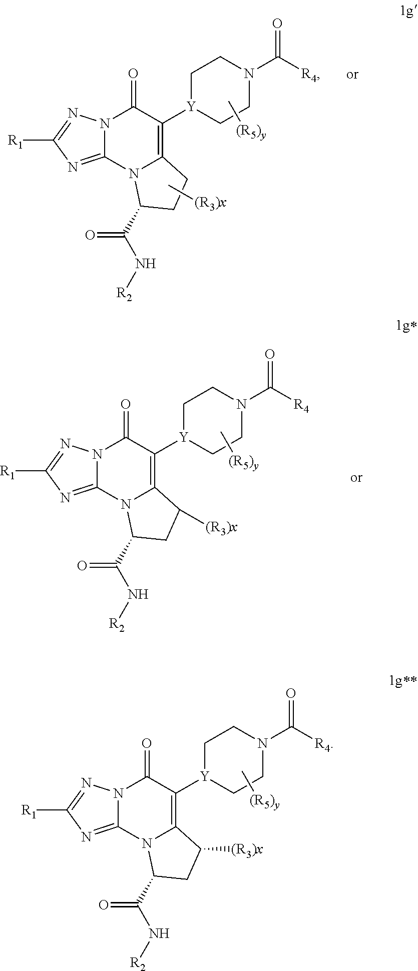

Embodiment 51. A compound of formula (I) or a pharmaceutically acceptable salt thereof, according to any of Embodiments 1 to 44 and 50, wherein formula (I) is formula 1h:

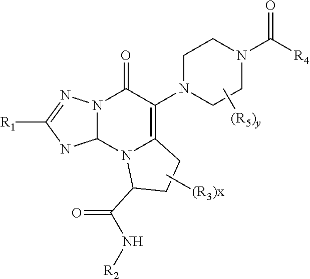

Most preferably, formula (I) is formula 1h.

There is also provided a compound of formula (I) which is formula 1h′:

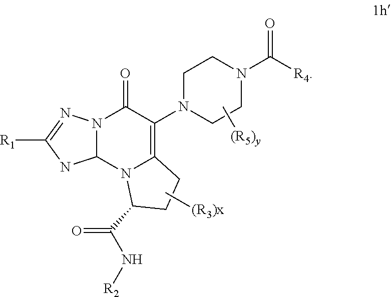

Embodiment 52. A compound of formula 1g, or 1g′, or a pharmaceutically acceptable salt thereof, according to embodiment 50,

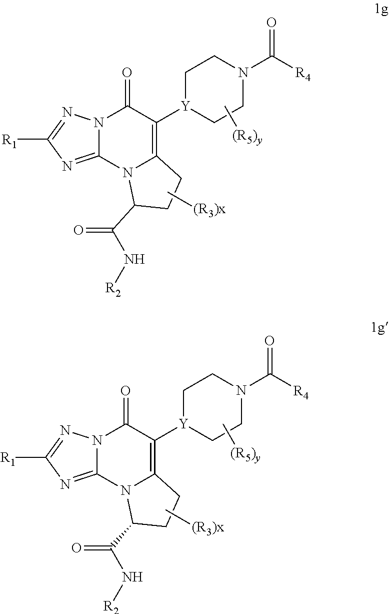

wherein R₁ is selected from:

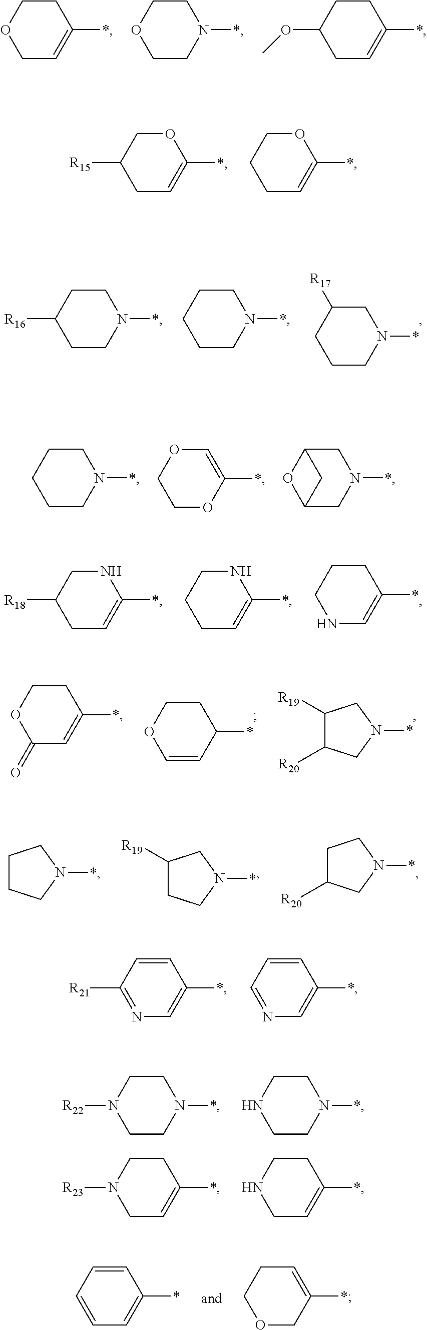

R₁₅ is F;
R₁₆ is R₂₅(R₂₄)N—;
R₁₇ is F;
R₁₈ is F;
R₁₉ is F;
R₂₀ is F;
R₂₁ is CH₃;
R₂₂ is CF₃, CHF₂CH₂, HOC(O)—CH₂—, H₃C—C(O)—, (H₃C)₃C—O—C(O)—;
R₂₃ is CF₃, CHF₂CH₂—, (H₃C)₃C—O—C(O)—;
R₂₄ is CH₃; and
R₂₅ is CHF₂CH₂—;
R₂ is the moiety:

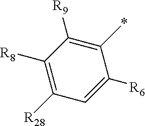

wherein
R₆ is selected from H, halo, (C₁-C₄)alkyl unsubstituted or substituted by 1, 2 or 3 halo;
R₈ is selected from H, halo, (C₁-C₄)alkyl unsubstituted or substituted by 1, 2 or 3 halo;
R₉ is selected from H, O—CH₃, OH, CN, CH₃ and halo;
R₂₈ is selected from SF₅, halo, (C₁-C₄)alkyl unsubstituted or substituted by 1, 2 or 3 halo and —C(O)H;
X is selected from C—R₇ and N; and
R₇ is selected from H and halo;
R₃ is (C₁-C₄)alkyl unsubstituted or substituted by 1, 2 or 3 substituents independently selected from halo and OH;
x is 0 or 1;
Y is CH or N, in particular N;
y is 0, 1 or 2;
R₅ is selected from CH₃;
or wherein in the moiety:

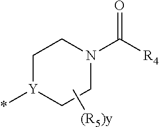

two R₅ substituents on adjacent carbon atoms join to form ring C:

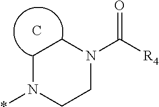

wherein ring C is a fused (C₃-C₆)cycloalkyl ring, in particular a fused cyclobutyl ring, and said fused (C₃-C₆)cycloalkyl ring is unsubstituted or substituted with 1 or 2 R₄₀ groups, wherein said R₄₀ is selected from:
   (C₁-C₂)alkyl, wherein each (C₁-C₂)alkyl is independently unsubstituted or substituted by OH or 1, 2 or 3 halo,
   halo, in particular F,
   or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
   or wherein two R₄₀ substituents on adjacent carbon atoms join together with the carbon atoms to which they are attached, to form a fused cyclopropyl ring;

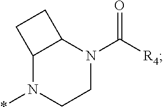

and in particular, is
R₄ is selected from:
CH₃,

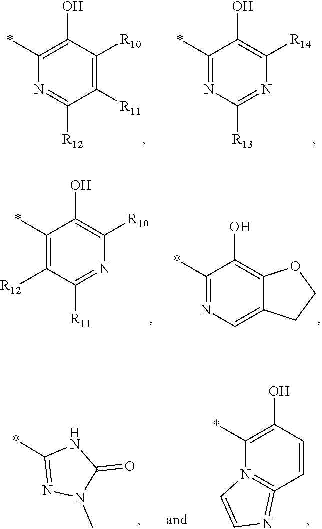

in particular

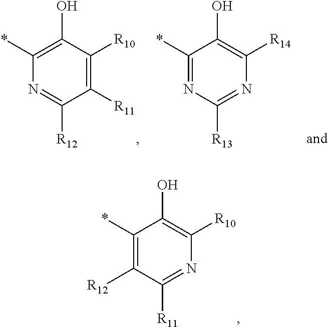

especially

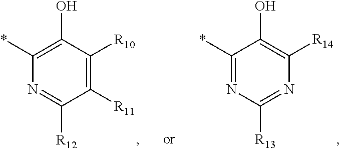

wherein
R₁₀ is selected from H, halo, (C₁-C₂)alkyl unsubstituted or substituted by 1, 2 or 3 halo substituents, —O—(C₁-C₂)alkyl unsubstituted or substituted by 1, 2 or 3 halo substituents;
R₁₁ is selected from H, halo, (C₁-C₂)alkyl unsubstituted or substituted by 1, 2 or 3 halo substituents;
R₁₂ is selected from H, halo, (C₁-C₂)alkyl unsubstituted or substituted by 1, 2 or 3 halo substituents;
R₁₃ is selected from H, —S—CH₃, halo, (C₁-C₂)alkyl unsubstituted or substituted by 1, 2 or 3 halo substituents; and
R₁₄ is selected from H, halo, (C₁-C₂)alkyl unsubstituted or substituted by 1, 2 or 3 halo substituents, O—(C₁-C₂)alkyl unsubstituted or substituted by 1, 2 or 3 halo substituents, and cyclopropyl.

In particular, the formula 1g is formula 1g* or formula 1g**.

Embodiment 53. A compound of formula 1 h or 1 h′ or a pharmaceutically acceptable salt thereof, according to embodiment 51,

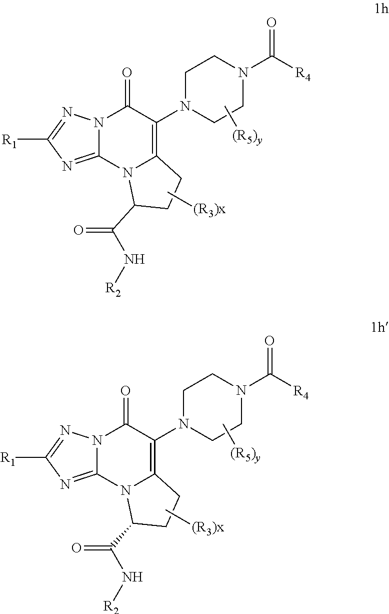

wherein:
R₁ is selected from:

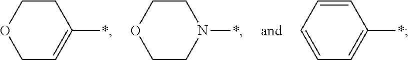

R₂ is selected from:

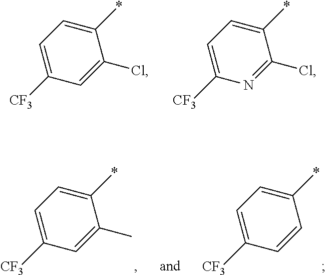 [SMILES: *c1ccc(C(F)(F)F)cc1Cl ; *c1ccc(C(F)(F)F)nc1Cl ; *c1ccc(C(F)(F)F)cc1C ; *c1ccc(C(F)(F)F)cc1]

R₃ is CH₃;
x is 0 or 1;
R₄ is selected from:

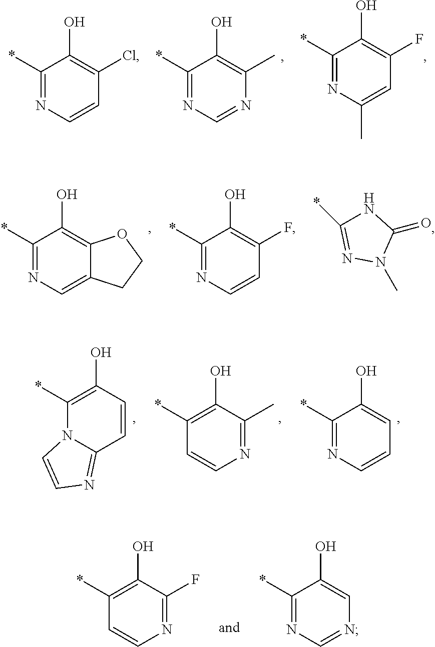

—CH₃,
y is 0 or 1;
R₅ is selected from CH₃;
or wherein the moiety:

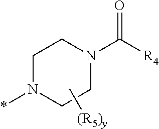

is

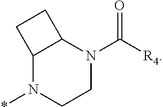

Embodiment 54. A compound of formula (I), or a pharmaceutically acceptable salt thereof, according to Embodiment 1, wherein the compound is selected from:

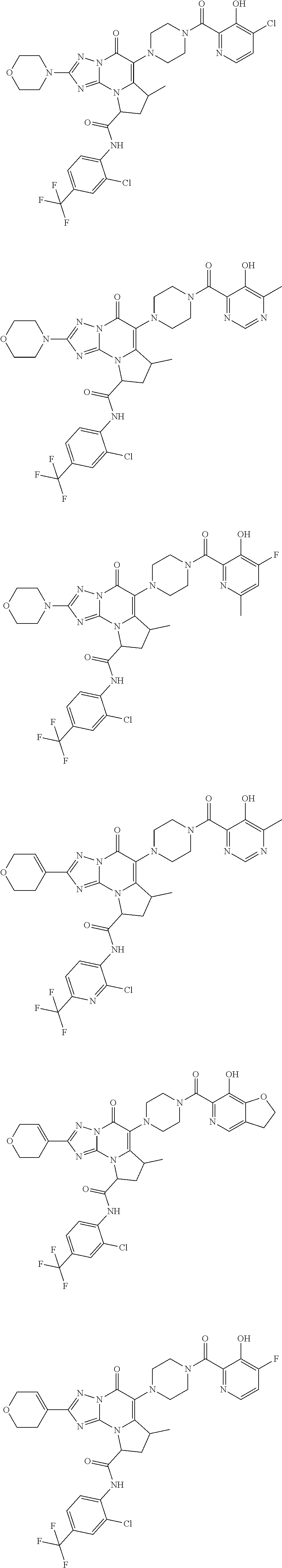

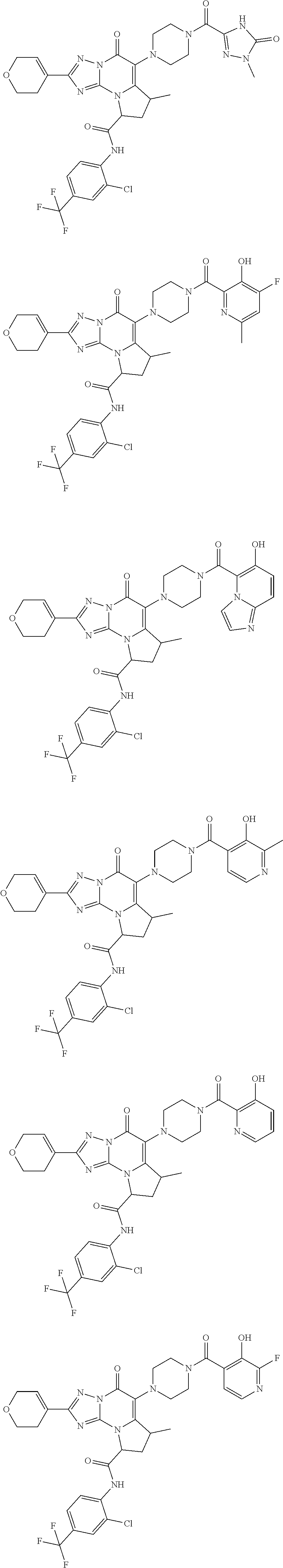

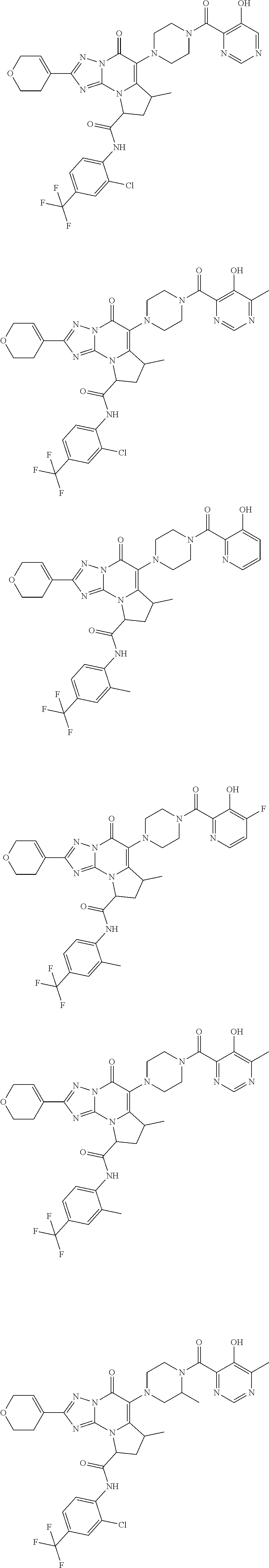

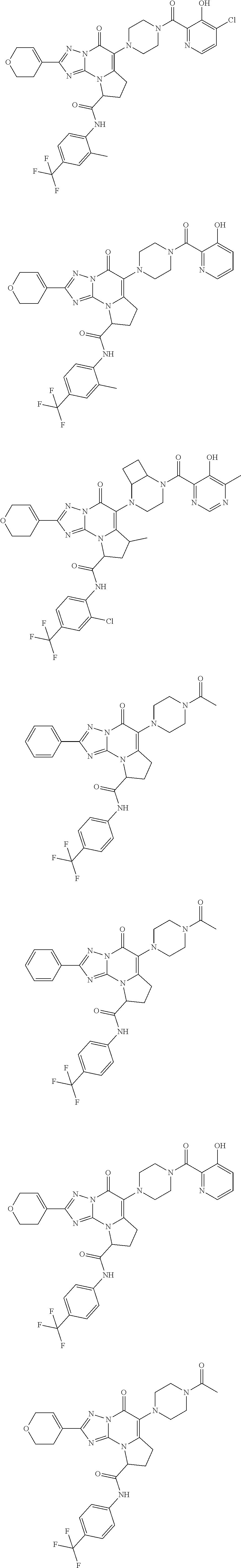

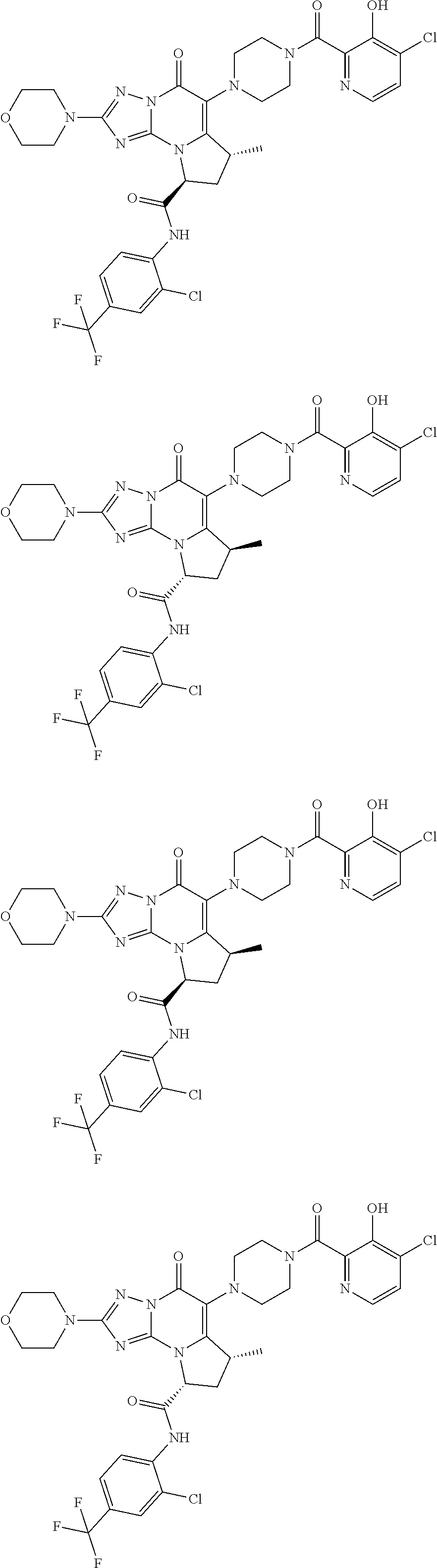 [SMILES: C[C@@H]1C[C@@H](C(=O)Nc2ccc(C(F)(F)F)cc2Cl)n2c1c(N1CCN(C(=O)c3nccc(Cl)c3O)CC1)c(=O)n1nc(N3CCOCC3)nc21 ; C[C@H]1C[C@H](C(=O)Nc2ccc(C(F)(F)F)cc2Cl)n2c1c(N1CCN(C(=O)c3nccc(Cl)c3O)CC1)c(=O)n1nc(N3CCOCC3)nc21 ; C[C@H]1C[C@@H](C(=O)Nc2ccc(C(F)(F)F)cc2Cl)n2c1c(N1CCN(C(=O)c3nccc(Cl)c3O)CC1)c(=O)n1nc(N3CCOCC3)nc21 ; C[C@@H]1C[C@H](C(=O)Nc2ccc(C(F)(F)F)cc2Cl)n2c1c(N1CCN(C(=O)c3nccc(Cl)c3O)CC1)c(=O)n1nc(N3CCOCC3)nc21]

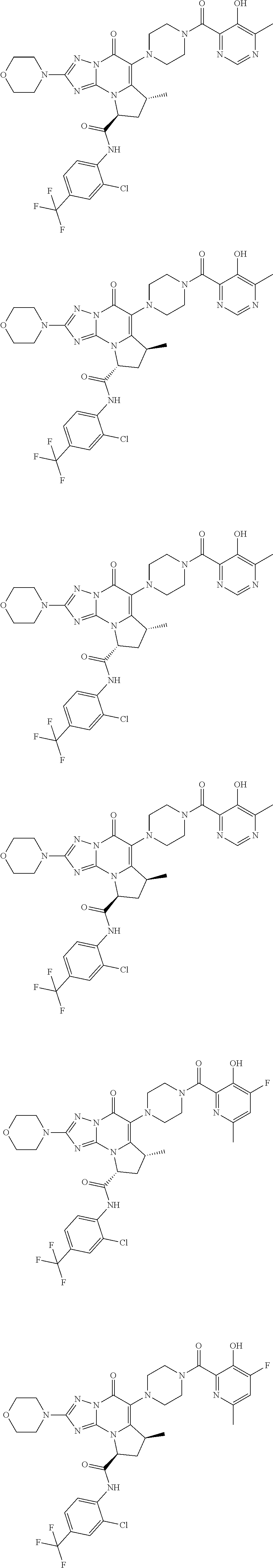

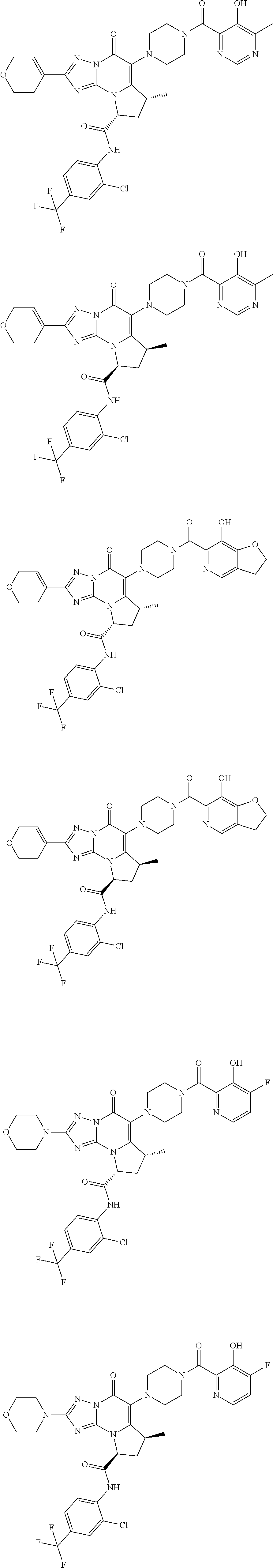

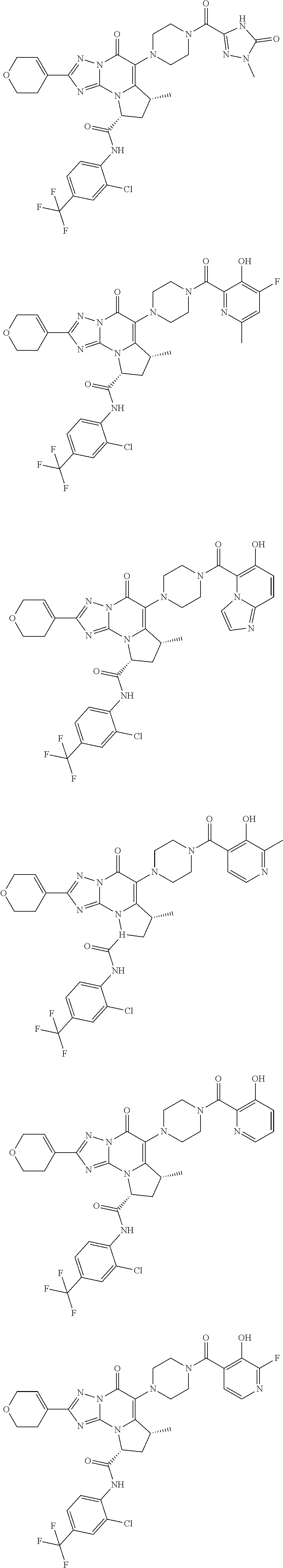

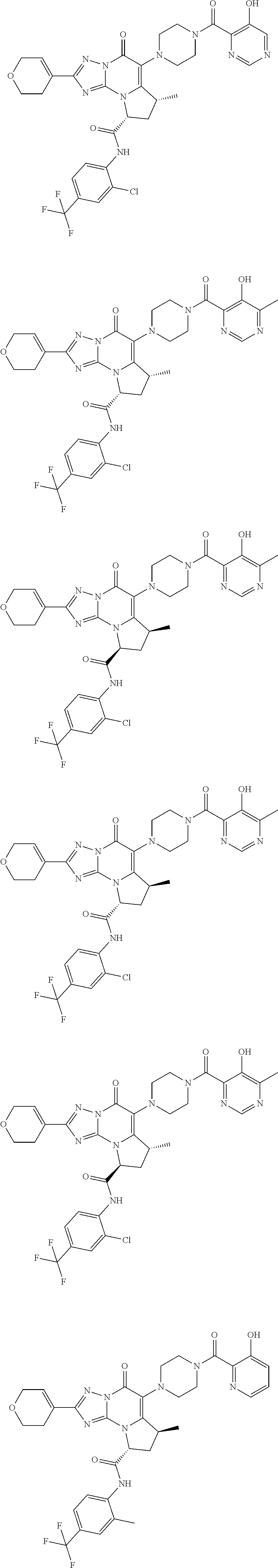

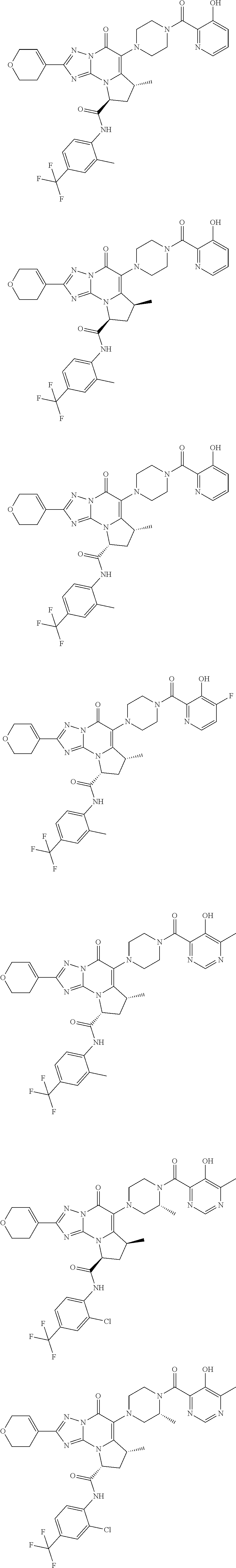

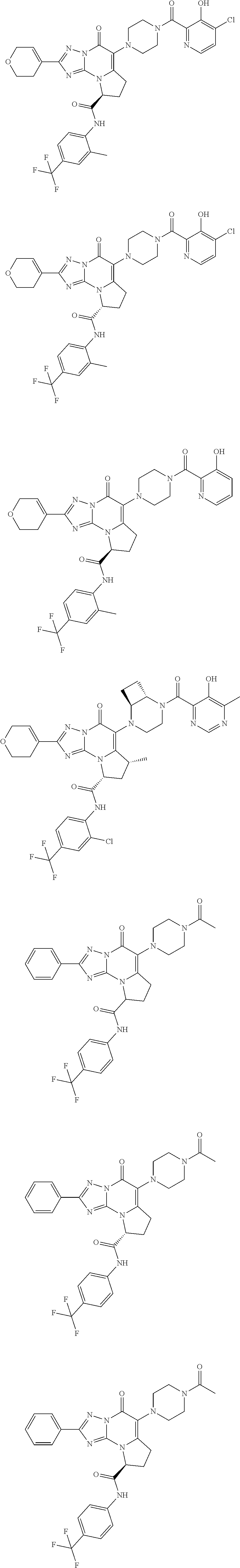

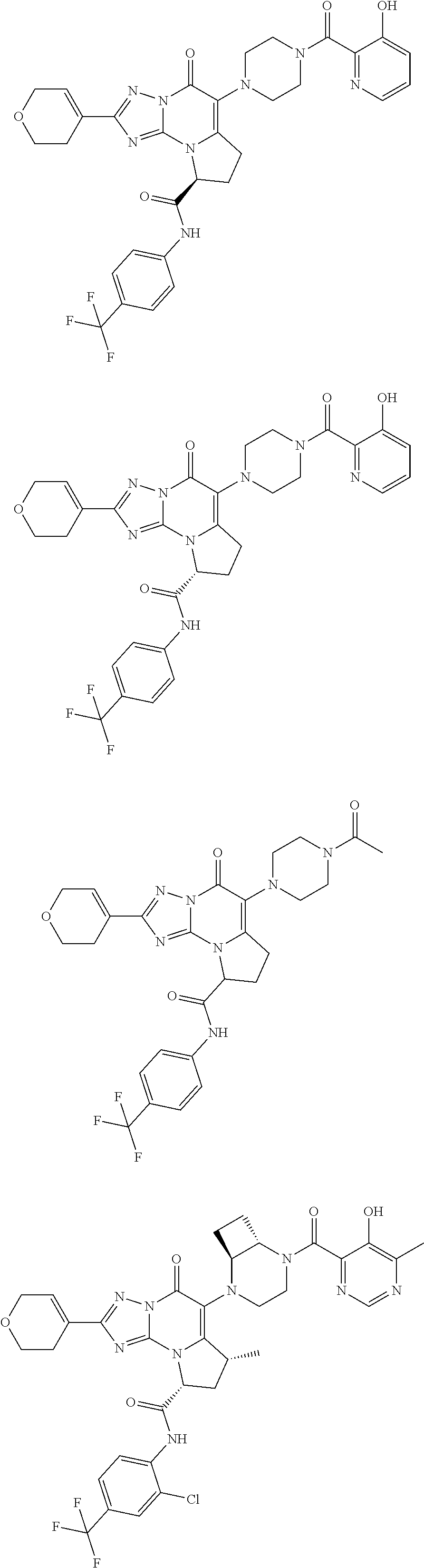 [SMILES: O=C(Nc1ccc(C(F)(F)F)cc1)[C@@H]1CCc2c(N3CCN(C(=O)c4ncccc4O)CC3)c(=O)n3nc(C4=CCOCC4)nc3n21 ; O=C(Nc1ccc(C(F)(F)F)cc1)[C@H]1CCc2c(N3CCN(C(=O)c4ncccc4O)CC3)c(=O)n3nc(C4=CCOCC4)nc3n21 ; CC(=O)N1CCN(c2c3n(c4nc(C5=CCOCC5)nn4c2=O)C(C(=O)Nc2ccc(C(F)(F)F)cc2)CC3)CC1 ; Cc1ncnc(C(=O)N2CCN(c3c4n(c5nc(C6=CCOCC6)nn5c3=O)[C@@H](C(=O)Nc3ccc(C(F)(F)F)cc3Cl)C[C@H]4C)[C@H]3CC[C@@H]32)c1O]

Embodiment 55. A compound of formula (I), according to Embodiment 1, wherein the compound is

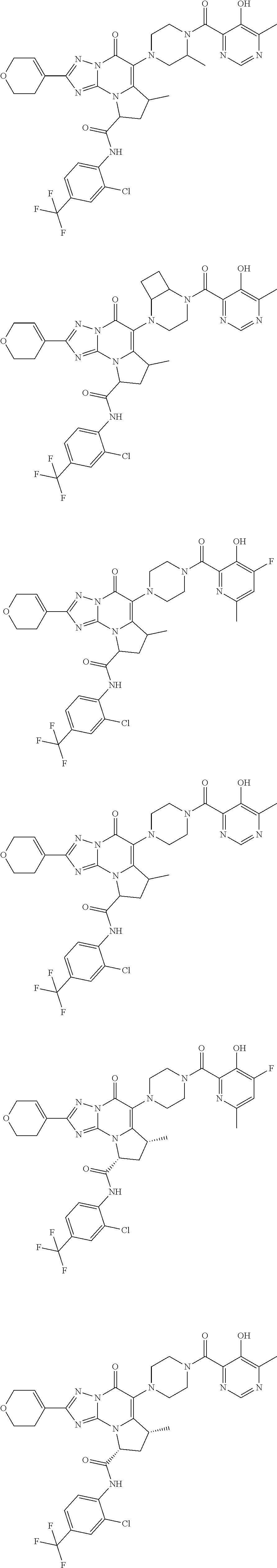

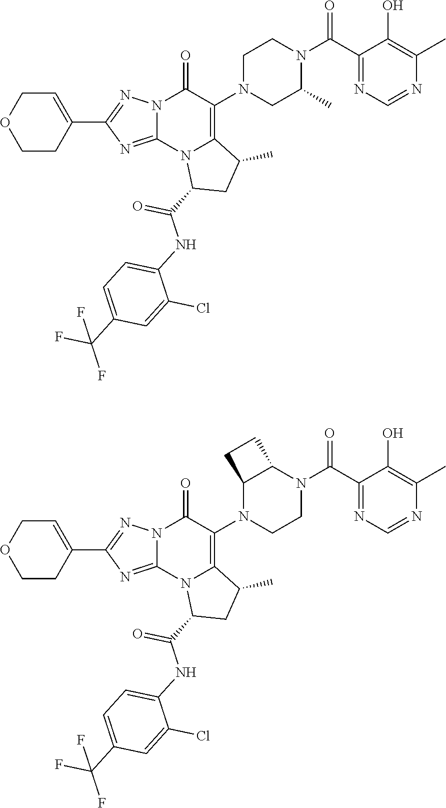 [SMILES: Cc1ncnc(C(=O)N2CCN(c3c4n(c5nc(C6=CCOCC6)nn5c3=O)[C@@H](C(=O)Nc3ccc(C(F)(F)F)cc3Cl)C[C@H]4C)C[C@H]2C)c1O ; Cc1ncnc(C(=O)N2CCN(c3c4n(c5nc(C6=CCOCC6)nn5c3=O)[C@@H](C(=O)Nc3ccc(C(F)(F)F)cc3Cl)C[C@H]4C)[C@H]3CC[C@@H]32)c1O]

Embodiment 56. A compound of formula (I), according to any of embodiments 1 to 44, 50, 51, 52, 53, 54 or 55, wherein the compound is (7R,9R)—N-(2-chloro-4-(trifluoromethyl)phenyl)-2-(3,6-dihydro-2H-pyran-4-yl)-6-((R)-4-(5-hydroxy-6-methylpyrimidine-4-carbonyl)-3-methylpiperazin-1-yl)-7-methyl-5-oxo-5,7,8,9-tetrahydropyrrolo[1,2-c][1,2,4]triazolo[1,5-a]pyrimidine-9-carboxamide, or a pharmaceutically acceptable salt thereof

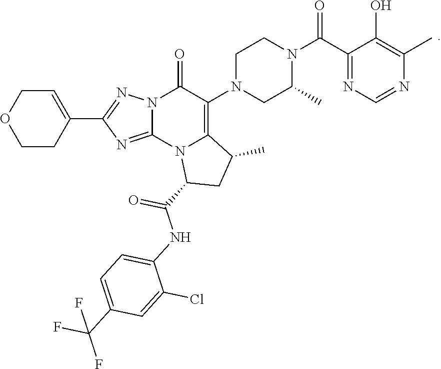 [SMILES: Cc1ncnc(C(=O)N2CCN(c3c4n(c5nc(C6=CCOCC6)nn5c3=O)[C@@H](C(=O)Nc3ccc(C(F)(F)F)cc3Cl)C[C@H]4C)C[C@H]2C)c1O]

Embodiment 57. A compound of formula (I), according to any of embodiments 1 to 44, 50, 51, 52, 53, 54 or 55, wherein the compound is (7R,9R)—N-(2-chloro-4-(trifluoromethyl)phenyl)-2-(3,6-dihydro-2H-pyran-4-yl)-6-((1S,6S)-5-(5-hydroxy-6-methylpyrimidine-4-carbonyl)-2,5-diazabicyclo[4.2.0]octan-2-yl)-7-methyl-5-oxo-5,7,8,9-tetrahydropyrrolo[1,2-c][1,2,4]triazolo[1,5-a]pyrimidine-9-carboxamide, or a pharmaceutically acceptable salt thereof

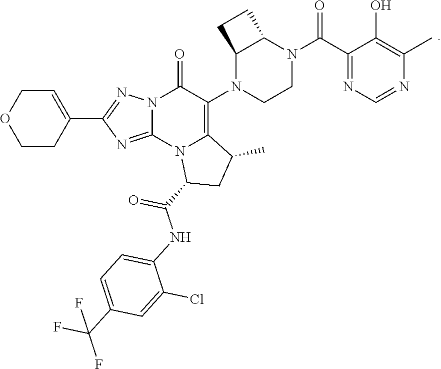 [SMILES: Cc1ncnc(C(=O)N2CCN(c3c4n(c5nc(C6=CCOCC6)nn5c3=O)[C@@H](C(=O)Nc3ccc(C(F)(F)F)cc3Cl)C[C@H]4C)[C@H]3CC[C@@H]32)c1O]

Embodiment 58. A compound of formula (1b) or a pharmaceutically acceptable salt thereof, according to any of Embodiments 1 to 43 or 45,

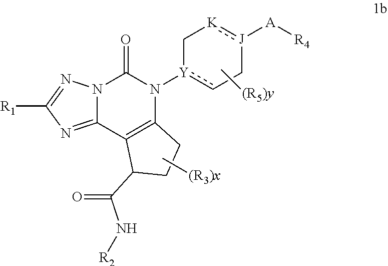

wherein R₁ is selected from:

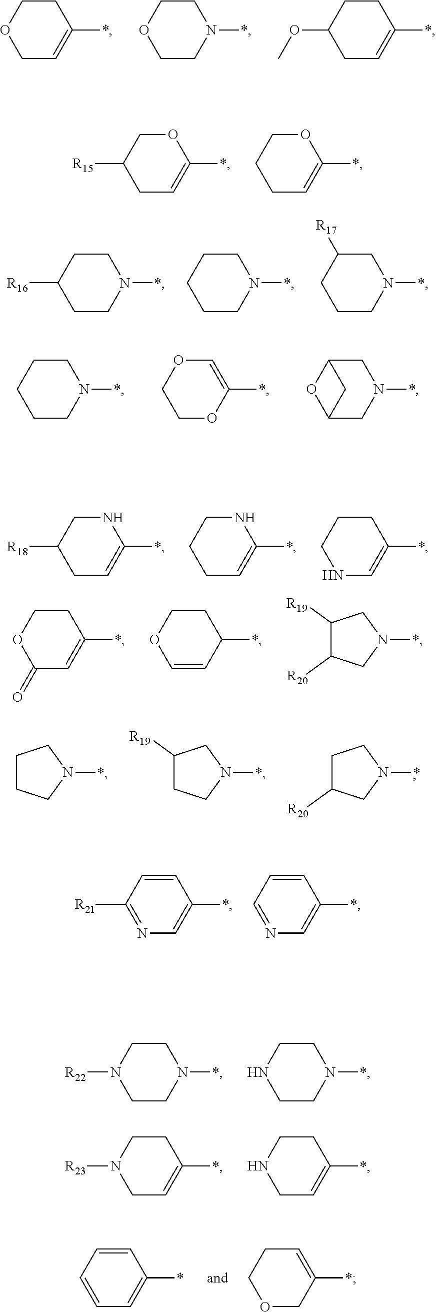

R₁₅ is F;
R₁₆ is R₂₅(R₂₄)N—;
R₁₇ is F;
R₁₈ is F;
R₁₉ is F;
R₂₀ is F;
R₂₁ is CH₃;
R₂₂ is CF₃, CHF₂CH₂, HOC(O)—CH₂—, H₃C—C(O)—, (H₃C)₃C—O—C(O)—;
R₂₃ is CF₃, CHF₂CH₂—, (H₃C)₃C—O—C(O)—;
R₂₄ is CH₃; and
R₂₅ is CHF₂CH₂—;
R₂ is the moiety:

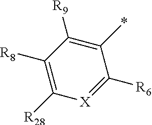

wherein
R₆ is selected from H, halo, (C₁-C₄)alkyl unsubstituted or substituted by 1, 2 or 3 halo;
R₈ is selected from H, halo, (C₁-C₄)alkyl unsubstituted or substituted by 1, 2 or 3 halo;
R₉ is selected from H, O—CH₃, OH, CN, CH₃ and halo;
R₂₈ is selected from SF₅, halo, (C₁-C₄)alkyl unsubstituted or substituted by 1, 2 or 3 halo and —C(O)H;
X is selected from C—R₇ and N; and
R₇ is selected from H and halo;
R₃ is (C₁-C₄)alkyl unsubstituted or substituted by 1, 2 or 3 substituents independently selected from halo and OH;
x is 0 or 1;
the moiety:

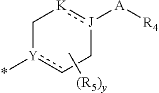

is as described in embodiment 1, or is in particular

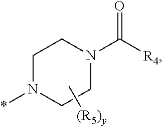

y is 0 or 1;
R₅ is selected from CH₃;
or wherein in the moiety:

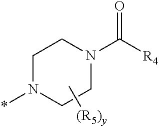

two R₅ substituents on adjacent carbon atoms join to form ring C:

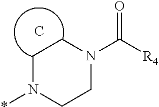

wherein ring C is a fused (C₃-C₆)cycloalkyl ring, in particular a fused cyclobutyl ring, and said fused (C₃-C₆)cycloalkyl ring is unsubstituted or substituted with 1 or 2 R₄₀ groups, wherein said R₄₀ is selected from:
   (C₁-C₂)alkyl, wherein each (C₁-C₂)alkyl is independently unsubstituted or substituted by OH or 1, 2 or 3 halo,
   halo, in particular F,
   or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
   or wherein two R₄₀ substituents on adjacent carbon atoms join together with the carbon atoms to which they are attached, to form a fused cyclopropyl ring;
and in particular, is

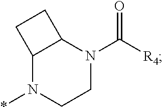

R₄ is selected from:
CH₃,

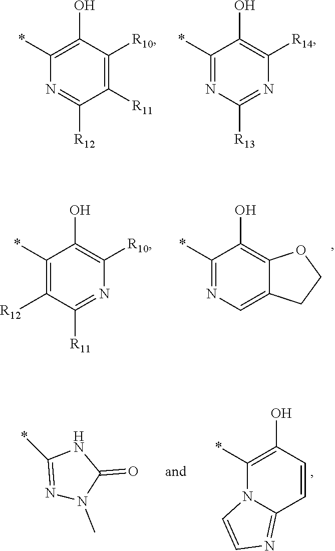

in particular

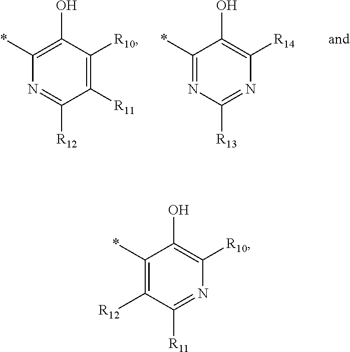

especially

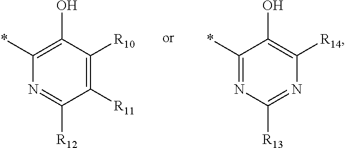

wherein
R₁₀ is selected from H, halo, (C₁-C₂)alkyl unsubstituted or substituted by 1, 2 or 3 halo substituents, —O—(C₁-C₂)alkyl unsubstituted or substituted by 1, 2 or 3 halo substituents;
R₁₁ is selected from H, halo, (C₁-C₂)alkyl unsubstituted or substituted by 1, 2 or 3 halo substituents;
R₁₂ is selected from H, halo, (C₁-C₂)alkyl unsubstituted or substituted by 1, 2 or 3 halo substituents;
R₁₃ is selected from H, —S—CH₃, halo, (C₁-C₂)alkyl unsubstituted or substituted by 1, 2 or 3 halo substituents; and
R₁₄ is selected from H, halo, (C₁-C₂)alkyl unsubstituted or substituted by 1, 2 or 3 halo substituents, O—(C₁-C₂)alkyl unsubstituted or substituted by 1, 2 or 3 halo substituents, and cyclopropyl.

Embodiment 59. A compound of formula (1 b) or a pharmaceutically acceptable salt thereof, according to any of Embodiments 1 to 43 or 45 or 58,

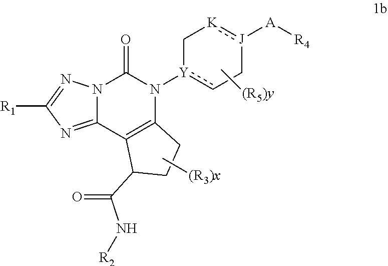

wherein:
R₁ is selected from:

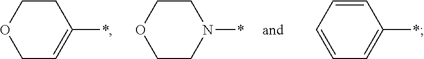

R₂ is selected from:

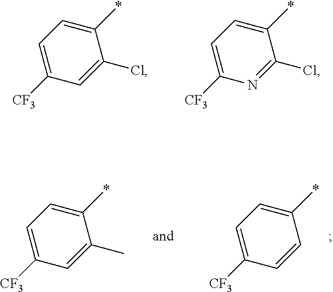 [SMILES: *c1ccc(C(F)(F)F)cc1Cl ; *c1ccc(C(F)(F)F)nc1Cl ; *c1ccc(C(F)(F)F)cc1C ; *c1ccc(C(F)(F)F)cc1]

R₃ is CH₃;
x is 0 or 1;
R₄ is selected from:
—CH₃,

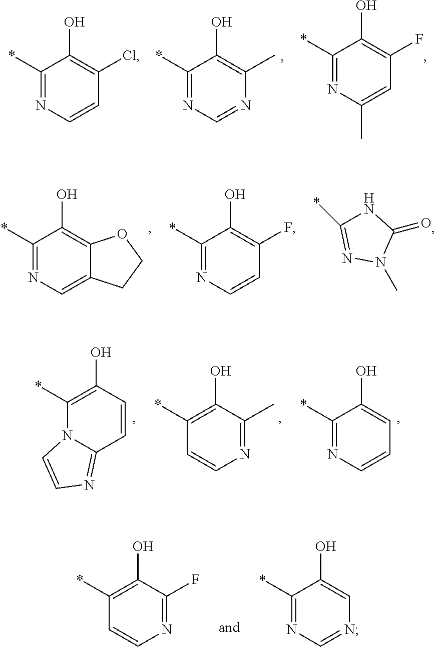

the moiety:

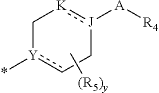

is:

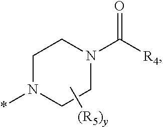

y is 0 or 1;
R₅ is selected from CH₃;
or wherein the moiety is

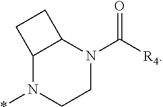

Embodiment 60. A compound of formula (1c) or a pharmaceutically acceptable salt thereof, according to any of Embodiments 1 to 43 or 46,

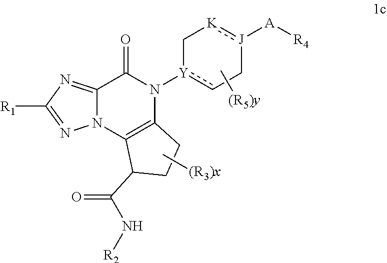

wherein R₁ is selected from:

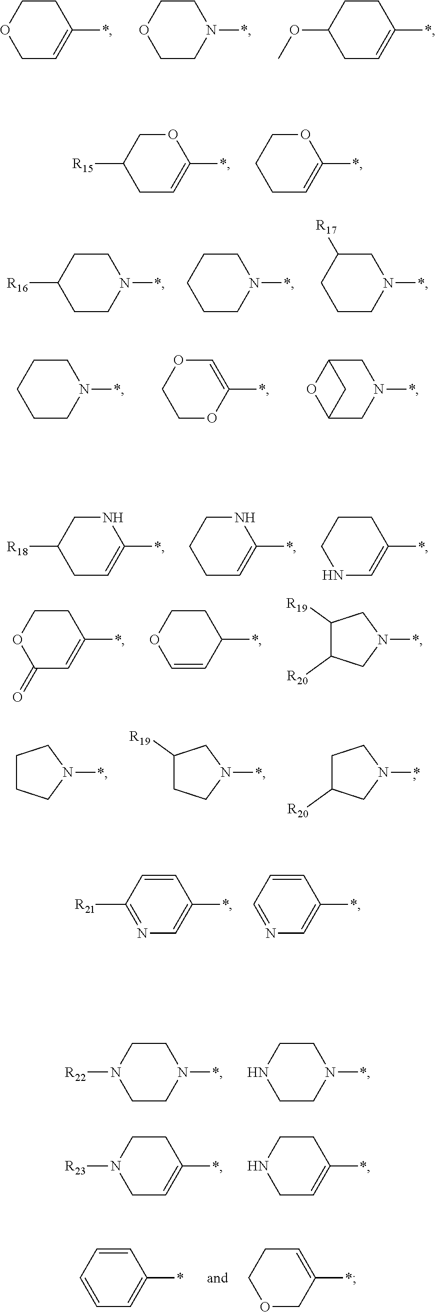

R₁₅ is F;
R₁₆ is R₂₅(R₂₄)N—;
R₁₇ is F;
R₁₈ is F;
R₁₉ is F;
R₂₀ is F;
R₂₁ is CH₃;
R₂₂ is CF₃, CHF₂CH₂, HOC(O)—CH₂—, H₃C—C(O)—, (H₃C)₃C—O—C(O)—;
R₂₃ is CF₃, CHF₂CH₂—, (H₃C)₃C—O—C(O)—;
R₂₄ is CH₃; and
R₂₅ is CHF₂CH₂—;
R₂ is the moiety:

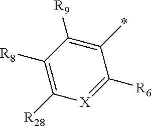

wherein
R₆ is selected from H, halo, (C₁-C₄)alkyl unsubstituted or substituted by 1, 2 or 3 halo;
R₈ is selected from H, halo, (C₁-C₄)alkyl unsubstituted or substituted by 1, 2 or 3 halo;
R₉ is selected from H, O—CH₃, OH, CN, CH₃ and halo;
R₂₈ is selected from SF₅, halo, (C₁-C₄)alkyl unsubstituted or substituted by 1, 2 or 3 halo and —C(O)H;
X is selected from C—R₇ and N; and
R₇ is selected from H and halo;
R₃ is (C₁-C₄)alkyl unsubstituted or substituted by 1, 2 or 3 substituents independently selected from halo and OH;
x is 0 or 1;
the moiety:

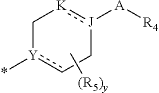

is as described in embodiment 1, or is in particular

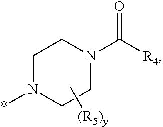

y is 0 or 1;
R₅ is selected from CH₃;
or wherein in the moiety:

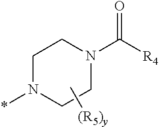

two R₅ substituents on adjacent carbon atoms join to form ring C:

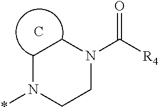

wherein ring C is a fused (C₃-C₆)cycloalkyl ring, in particular a fused cyclobutyl ring, and said fused (C₃-C₆)cycloalkyl ring is unsubstituted or substituted with 1 or 2 R₄₀ groups, wherein said R₄₀ is selected from:
   (C₁-C₂)alkyl, wherein each (C₁-C₂)alkyl is independently unsubstituted or substituted by OH or 1, 2 or 3 halo,
   halo, in particular F,
   or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
   or wherein two R₄₀ substituents on adjacent carbon atoms join together with the carbon atoms to which they are attached, to form a fused cyclopropyl ring;
and in particular, is

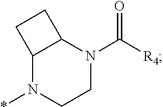

R₄ is selected from:
CH₃,

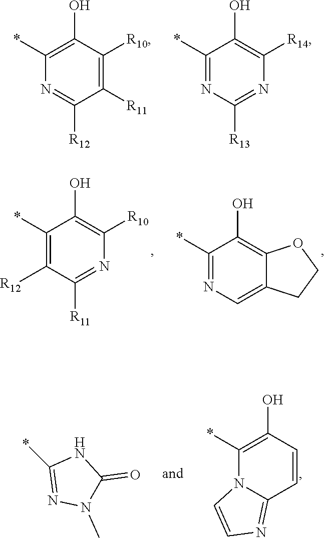

in particular

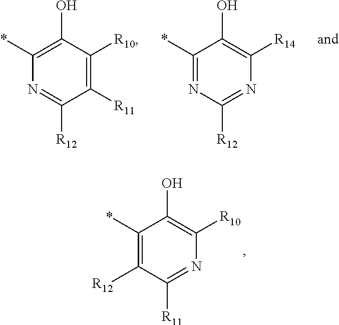

especially

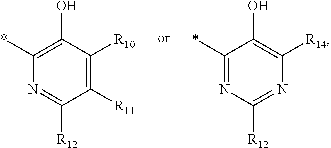

wherein
R₁₀ is selected from H, halo, (C₁-C₂)alkyl unsubstituted or substituted by 1, 2 or 3 halo substituents, —O—(C₁-C₂)alkyl unsubstituted or substituted by 1, 2 or 3 halo substituents;
R₁₁ is selected from H, halo, (C₁-C₂)alkyl unsubstituted or substituted by 1, 2 or 3 halo substituents;
R₁₂ is selected from H, halo, (C₁-C₂)alkyl unsubstituted or substituted by 1, 2 or 3 halo substituents;
R₁₃ is selected from H, —S—CH₃, halo, (C₁-C₂)alkyl unsubstituted or substituted by 1, 2 or 3 halo substituents; and
R₁₄ is selected from H, halo, (C₁-C₂)alkyl unsubstituted or substituted by 1, 2 or 3 halo substituents, O—(C₁-C₂)alkyl unsubstituted or substituted by 1, 2 or 3 halo substituents, and cyclopropyl.

Embodiment 61. A compound of formula (1c) or a pharmaceutically acceptable salt thereof, according to any of Embodiments 1 to 43 or 46 or 60,

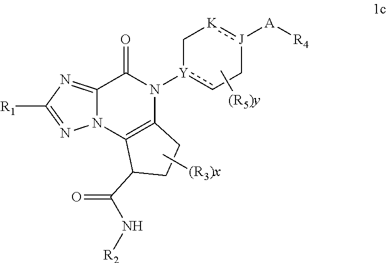

wherein:
R₁ is selected from:

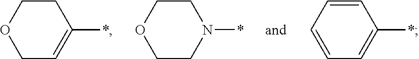

R₂ is selected from:

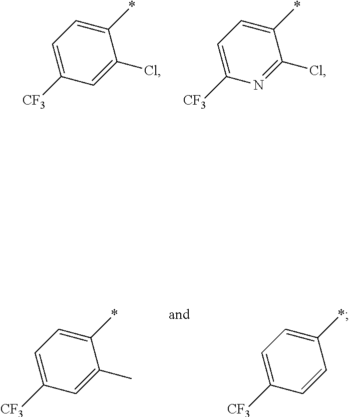

R₃ is CH₃;
x is 0 or 1;
R₄ is selected from:
—CH₃,

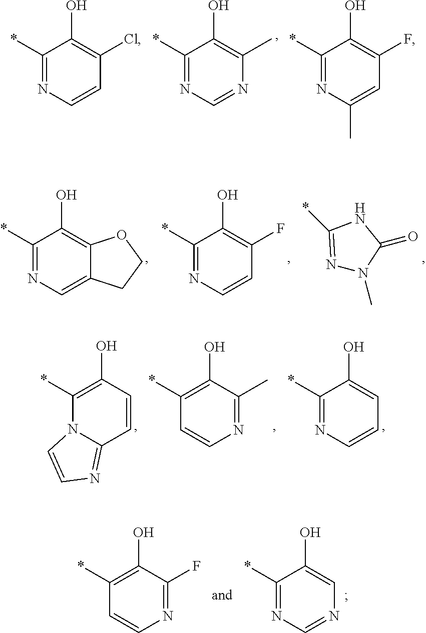

the moiety:

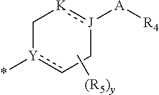

is:

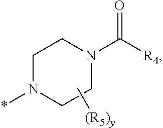

y is 0 or 1;
R₅ is selected from CH₃;
or wherein the moiety is

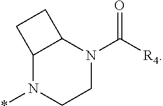

Embodiment 62. A compound of formula (1d) or a pharmaceutically acceptable salt thereof, according to any of Embodiments 1 to 43 or 47,

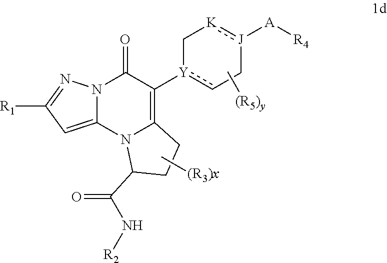

wherein R₁ is selected from:

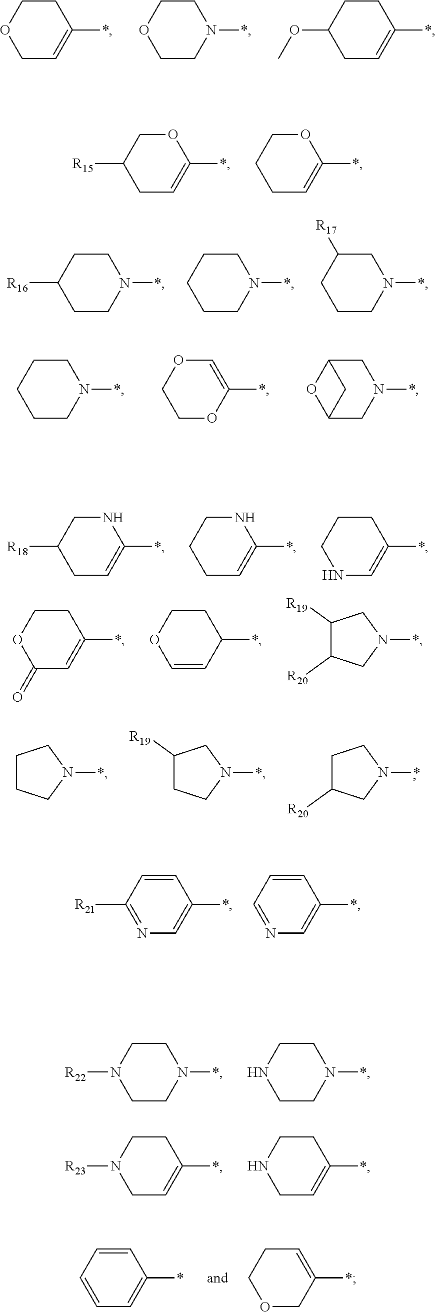

R₁₅ is F;
R₁₆ is R₂₅(R₂₄)N—;
R₁₇ is F;
R₁₈ is F;
R₁₉ is F;
R₂₀ is F;
R₂₁ is CH₃;
R₂₂ is CF₃, CHF₂CH₂, HOC(O)—CH₂—, H₃C—C(O)—, (H₃C)₃C—O—C(O)—;
R₂₃ is CF₃, CHF₂CH₂—, (H₃C)₃C—O—C(O)—;
R₂₄ is CH₃; and
R₂₅ is CHF₂CH₂—;
R₂ is the moiety:

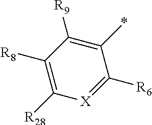

wherein
R₆ is selected from H, halo, (C₁-C₄)alkyl unsubstituted or substituted by 1, 2 or 3 halo;
R₈ is selected from H, halo, (C₁-C₄)alkyl unsubstituted or substituted by 1, 2 or 3 halo;
R₉ is selected from H, O—CH₃, OH, CN, CH₃ and halo;
R₂₈ is selected from SF₅, halo, (C₁-C₄)alkyl unsubstituted or substituted by 1, 2 or 3 halo and —C(O)H;
X is selected from C—R₇ and N; and
R₇ is selected from H and halo;
R₃ is (C₁-C₄)alkyl unsubstituted or substituted by 1, 2 or 3 substituents independently selected from halo and OH;
x is 0 or 1;
the moiety:

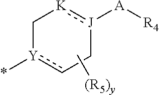

is as described in embodiment 1, or is in particular

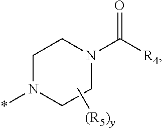

y is 0 or 1;
R₅ is selected from CH₃;
or wherein in the moiety:

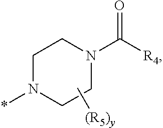

two R₅ substituents on adjacent carbon atoms join to form ring C:

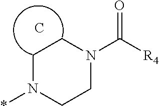

wherein ring C is a fused (C₃-C₆)cycloalkyl ring, in particular a fused cyclobutyl ring, and said fused (C₃-C₆)cycloalkyl ring is unsubstituted or substituted with 1 or 2 R₄₀ groups, wherein said R₄₀ is selected from:
   (C₁-C₂)alkyl, wherein each (C₁-C₂)alkyl is independently unsubstituted or substituted by OH or 1, 2 or 3 halo,
   halo, in particular F,
   or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
   or wherein two R₄₀ substituents on adjacent carbon atoms join together with the carbon atoms to which they are attached, to form a fused cyclopropyl ring;
and in particular, is

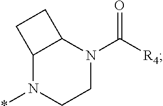

R₄ is selected from:
CH₃,

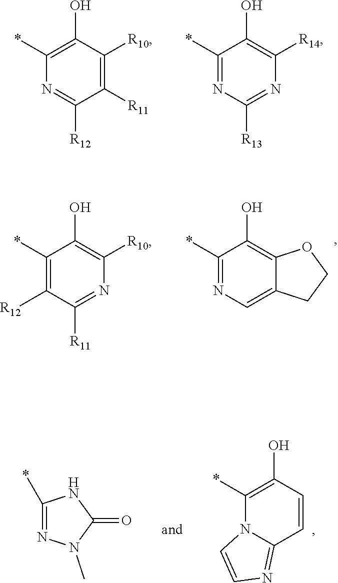

in particular

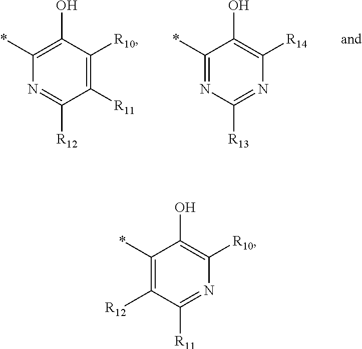

especially

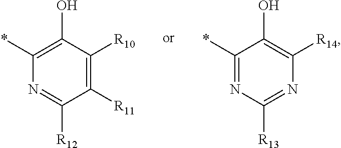

wherein
R₁₀ is selected from H, halo, (C₁-C₂)alkyl unsubstituted or substituted by 1, 2 or 3 halo substituents, —O—(C₁-C₂)alkyl unsubstituted or substituted by 1, 2 or 3 halo substituents;
R₁₁ is selected from H, halo, (C₁-C₂)alkyl unsubstituted or substituted by 1, 2 or 3 halo substituents;
R₁₂ is selected from H, halo, (C₁-C₂)alkyl unsubstituted or substituted by 1, 2 or 3 halo substituents;
R₁₃ is selected from H, —S—CH₃, halo, (C₁-C₂)alkyl unsubstituted or substituted by 1, 2 or 3 halo substituents; and
R₁₄ is selected from H, halo, (C₁-C₂)alkyl unsubstituted or substituted by 1, 2 or 3 halo substituents, O—(C₁-C₂)alkyl unsubstituted or substituted by 1, 2 or 3 halo substituents, and cyclopropyl.

Embodiment 63. A compound of formula (1d) or a pharmaceutically acceptable salt thereof, according to any of Embodiments 1 to 43 or 47 or 62,

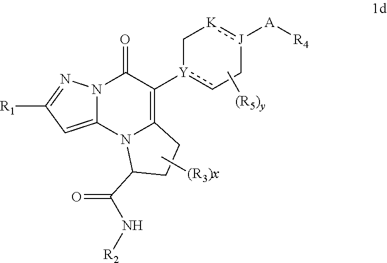

wherein:
R₁ is selected from:

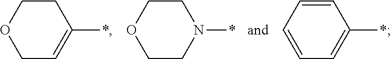

R₂ is selected from:

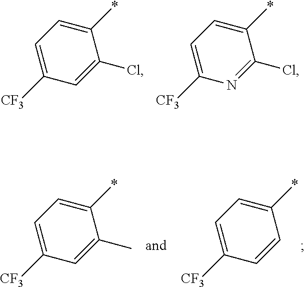 [SMILES: *c1ccc(C(F)(F)F)cc1Cl ; *c1ccc(C(F)(F)F)nc1Cl ; *c1ccc(C(F)(F)F)cc1C ; *c1ccc(C(F)(F)F)cc1]

R₃ is CH₃;
x is 0 or 1;
R₄ is selected from:
—CH₃,

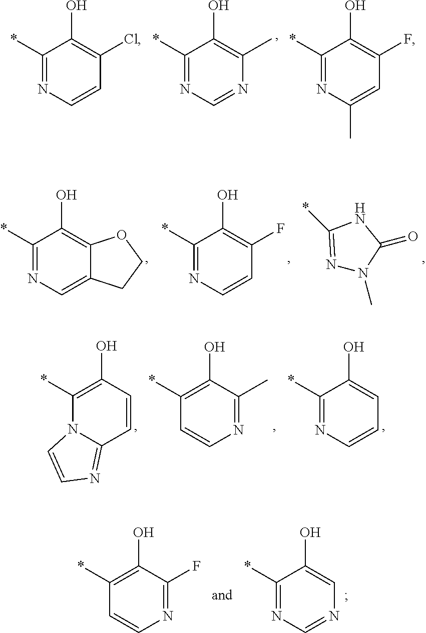

the moiety:

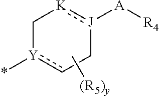

is:

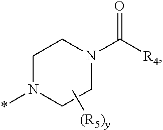

y is 0 or 1;
R₅ is selected from CH₃;
or wherein the moiety is

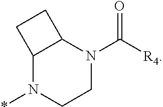

Embodiment 64. A compound of formula (1e) or a pharmaceutically acceptable salt thereof, according to any of Embodiments 1 to 43 or 48,

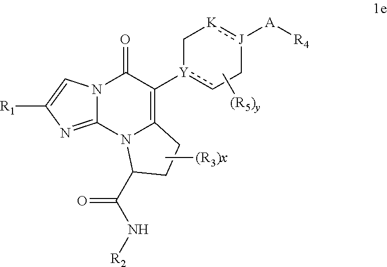

wherein R₁ is selected from:

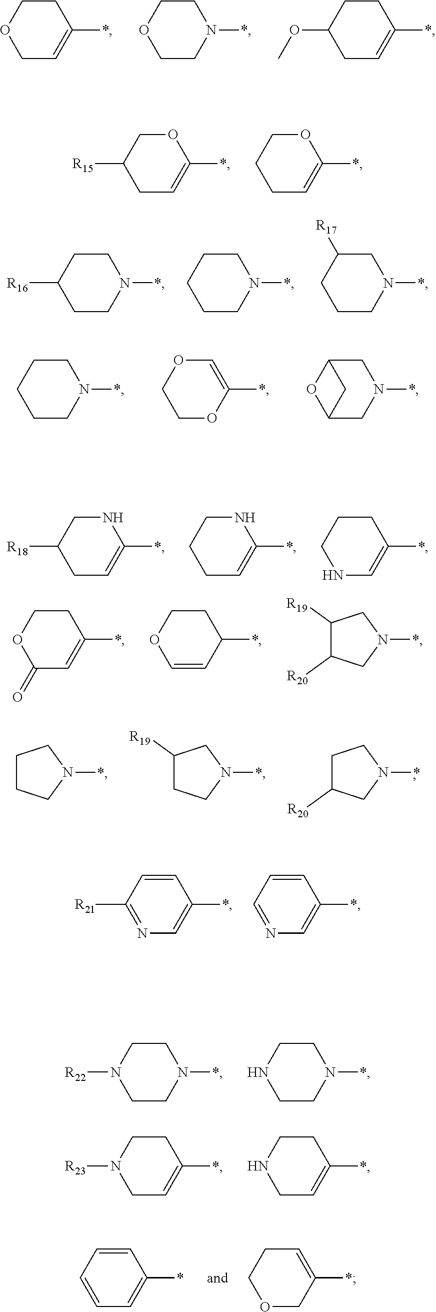

R₁₅ is F;
R₁₆ is R₂₅(R₂₄)N—;
R₁₇ is F;
R₁₈ is F;
R₁₉ is F;
R₂₀ is F;
R₂₁ is CH₃;
R₂₂ is CF₃, CHF₂CH₂, HOC(O)—CH₂—, H₃C—C(O)—, (H₃C)₃C—O—C(O)—;
R₂₃ is CF₃, CHF₂CH₂—, (H₃C)₃C—O—C(O)—;
R₂₄ is CH₃; and
R₂₅ is CHF₂CH₂—;
R₂ is the moiety:

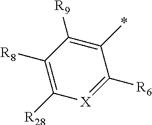

wherein
R₆ is selected from H, halo, (C₁-C₄)alkyl unsubstituted or substituted by 1, 2 or 3 halo;
R₈ is selected from H, halo, (C₁-C₄)alkyl unsubstituted or substituted by 1, 2 or 3 halo;
R₉ is selected from H, O—CH₃, OH, CN, CH₃ and halo;
R₂₈ is selected from SF₅, halo, (C₁-C₄)alkyl unsubstituted or substituted by 1, 2 or 3 halo and —C(O)H;
X is selected from C—R₇ and N; and
R₇ is selected from H and halo;
R₃ is (C₁-C₄)alkyl unsubstituted or substituted by 1, 2 or 3 substituents independently selected from halo and OH;
x is 0 or 1;
the moiety:

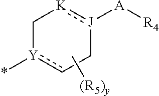

is as described in embodiment 1, or is in particular

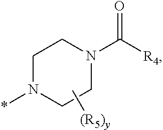

y is 0 or 1;
R₅ is selected from CH₃;
or wherein in the moiety:

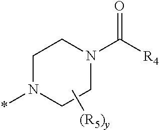

two R₅ substituents on adjacent carbon atoms join to form ring C:

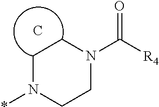

wherein ring C is a fused (C₃-C₆)cycloalkyl ring, in particular a fused cyclobutyl ring, and said fused (C₃-C₆)cycloalkyl ring is unsubstituted or substituted with 1 or 2 R₄₀ groups, wherein said R₄₀ is selected from:
   (C₁-C₂)alkyl, wherein each (C₁-C₂)alkyl is independently unsubstituted or substituted by OH or 1, 2 or 3 halo,
   halo, in particular F,
   or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
   or wherein two R₄₀ substituents on adjacent carbon atoms join together with the carbon atoms to which they are attached, to form a fused cyclopropyl ring;
and in particular, is

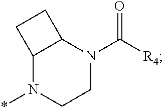

R₄ is selected from:
CH₃,

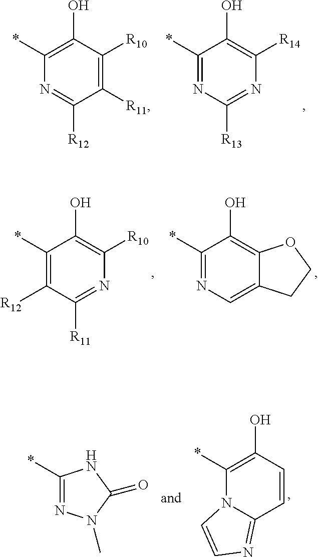

in particular

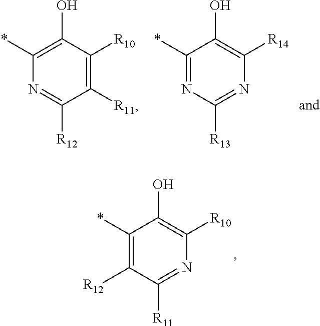

especially

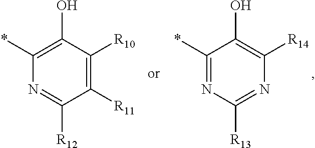

wherein
R₁₀ is selected from H, halo, (C₁-C₂)alkyl unsubstituted or substituted by 1, 2 or 3 halo substituents, —O—(C₁-C₂)alkyl unsubstituted or substituted by 1, 2 or 3 halo substituents;
R₁₁ is selected from H, halo, (C₁-C₂)alkyl unsubstituted or substituted by 1, 2 or 3 halo substituents;
R₁₂ is selected from H, halo, (C₁-C₂)alkyl unsubstituted or substituted by 1, 2 or 3 halo substituents;
R₁₃ is selected from H, —S—CH₃, halo, (C₁-C₂)alkyl unsubstituted or substituted by 1, 2 or 3 halo substituents; and
R₁₄ is selected from H, halo, (C₁-C₂)alkyl unsubstituted or substituted by 1, 2 or 3 halo substituents, O—(C₁-C₂)alkyl unsubstituted or substituted by 1, 2 or 3 halo substituents, and cyclopropyl.

Embodiment 65. A compound of formula (1e) or a pharmaceutically acceptable salt thereof, according to any of Embodiments 1 to 43 or 48 or 64,

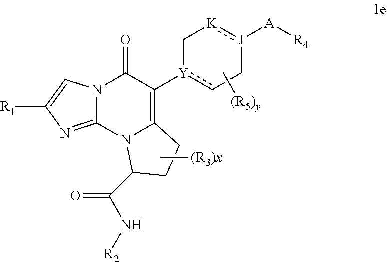

wherein:
R₁ is selected from:

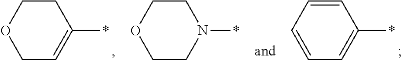

R₂ is selected from:

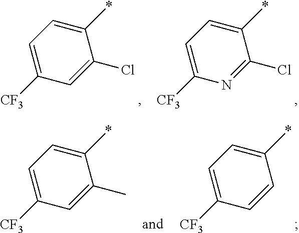 [SMILES: *c1ccc(C(F)(F)F)cc1Cl ; *c1ccc(C(F)(F)F)nc1Cl ; *c1ccc(C(F)(F)F)cc1C ; *c1ccc(C(F)(F)F)cc1]

R₃ is CH₃;
x is 0 or 1;
R₄ is selected from:
—CH₃,

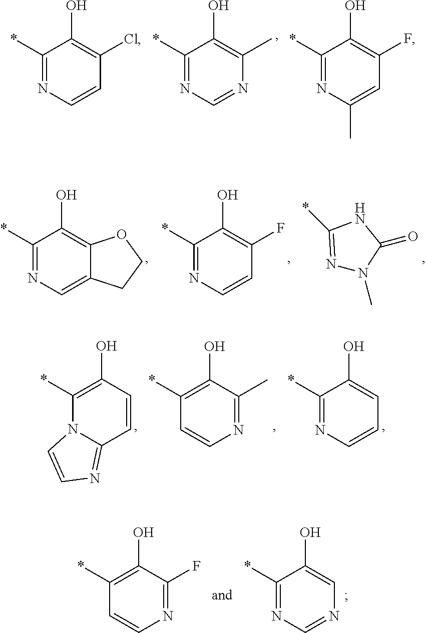

the moiety:

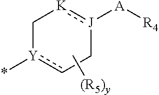

is:

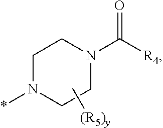

y is 0 or 1;
R₅ is selected from CH₃;
or wherein the moiety is

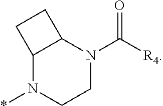

Embodiment 66. A compound of formula (1f) or a pharmaceutically acceptable salt thereof, according to any of Embodiments 1 to 43 or 49,

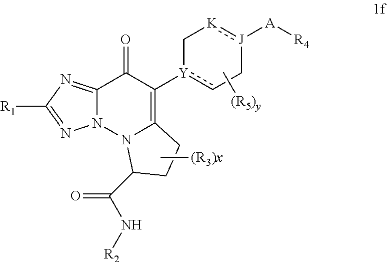

wherein R₁ is selected from:

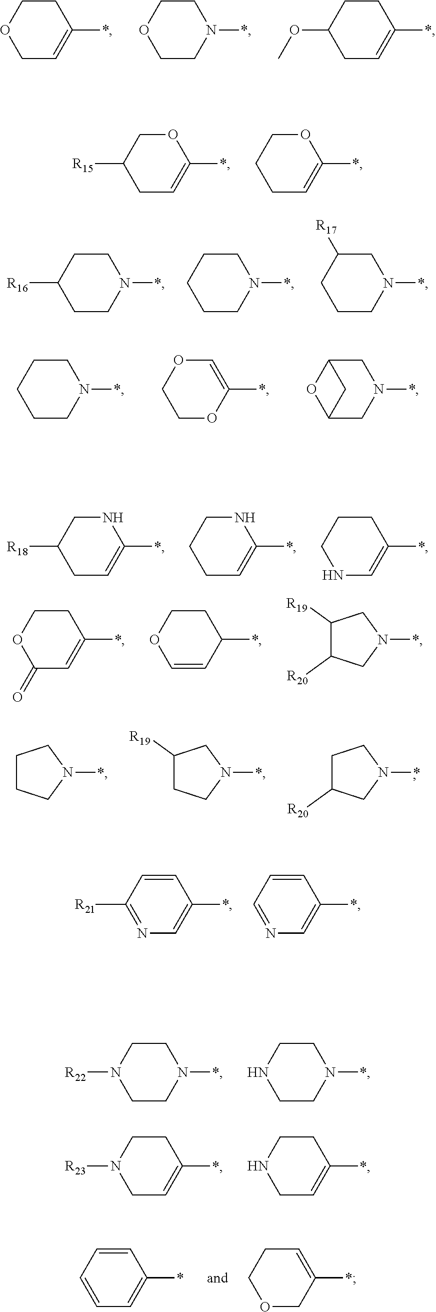

R₁₅ is F;
R₁₆ is R₂₅(R₂₄)N—;
R₁₇ is F;
R₁₈ is F;
R₁₉ is F;
R₂₀ is F;
R₂₁ is CH₃;
R₂₂ is CF₃, CHF₂CH₂, HOC(O)—CH₂—, H₃C—C(O)—, (H₃C)₃C—O—C(O)—;
R₂₃ is CF₃, CHF₂CH₂—, (H₃C)₃C—O—C(O)—;
R₂₄ is CH₃; and
R₂₅ is CHF₂CH₂—;
R₂ is the moiety:

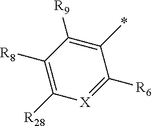

wherein R₆ is selected from H, halo, (C₁-C₄)alkyl unsubstituted or substituted by 1, 2 or 3 halo;
R₈ is selected from H, halo, (C₁-C₄)alkyl unsubstituted or substituted by 1, 2 or 3 halo;
R₉ is selected from H, O—CH₃, OH, CN, CH₃ and halo;
R₂₈ is selected from SF₅, halo, (C₁-C₄)alkyl unsubstituted or substituted by 1, 2 or 3 halo and —C(O)H;
X is selected from C—R₇ and N; and
R₇ is selected from H and halo;
R₃ is (C₁-C₄)alkyl unsubstituted or substituted by 1, 2 or 3 substituents independently selected from halo and OH;
x is 0 or 1;
the moiety:

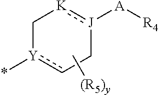

is as described in embodiment 1, or is in particular

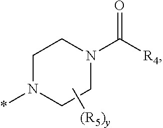

y is 0 or 1;
R₅ is selected from CH₃;
or wherein in the moiety:

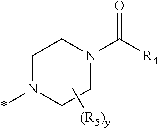

two R₅ substituents on adjacent carbon atoms join to form ring C:

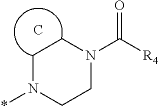

wherein ring C is a fused (C₃-C₆)cycloalkyl ring, in particular a fused cyclobutyl ring, and said fused (C₃-C₆)cycloalkyl ring is unsubstituted or substituted with 1 or 2 R₄₀ groups, wherein said R₄₀ is selected from:
   (C₁-C₂)alkyl, wherein each (C₁-C₂)alkyl is independently unsubstituted or substituted by OH or 1, 2 or 3 halo,
   halo, in particular F,
   or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
   or wherein two R₄₀ substituents on adjacent carbon atoms join together with the carbon atoms to which they are attached, to form a fused cyclopropyl ring;
and in particular, is

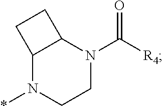

R₄ is selected from:
CH₃,

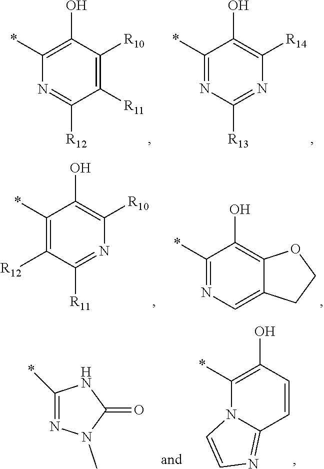

in particular

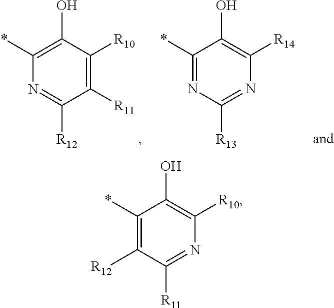

especially

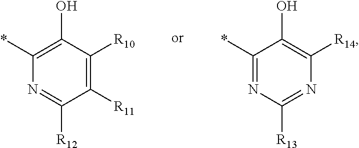

wherein
R₁₀ is selected from H, halo, (C₁-C₂)alkyl unsubstituted or substituted by 1, 2 or 3 halo substituents, —O—(C₁-C₂)alkyl unsubstituted or substituted by 1, 2 or 3 halo substituents;
R₁₁ is selected from H, halo, (C₁-C₂)alkyl unsubstituted or substituted by 1, 2 or 3 halo substituents;
R₁₂ is selected from H, halo, (C₁-C₂)alkyl unsubstituted or substituted by 1, 2 or 3 halo substituents;
R₁₃ is selected from H, —S—CH₃, halo, (C₁-C₂)alkyl unsubstituted or substituted by 1, 2 or 3 halo substituents; and
R₁₄ is selected from H, halo, (C₁-C₂)alkyl unsubstituted or substituted by 1, 2 or 3 halo substituents, O—(C₁-C₂)alkyl unsubstituted or substituted by 1, 2 or 3 halo substituents, and cyclopropyl.

Embodiment 67. A compound of formula (1f) or a pharmaceutically acceptable salt thereof, according to any of Embodiments 1 to 43 or 49 or 66,

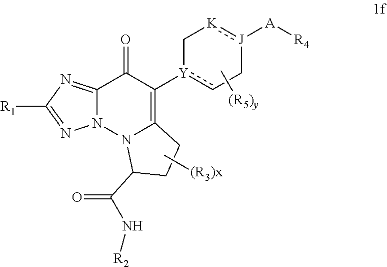

wherein:
R₁ is selected from:

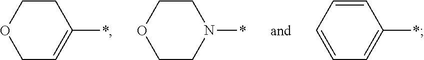

R₂ is selected from:

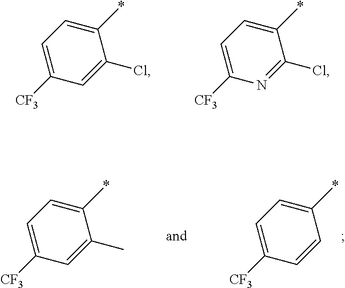 [SMILES: *c1ccc(C(F)(F)F)cc1Cl ; *c1ccc(C(F)(F)F)nc1Cl ; *c1ccc(C(F)(F)F)cc1C ; *c1ccc(C(F)(F)F)cc1]

R₃ is CH₃;
x is 0 or 1;
R₄ is selected from:
—CH₃,

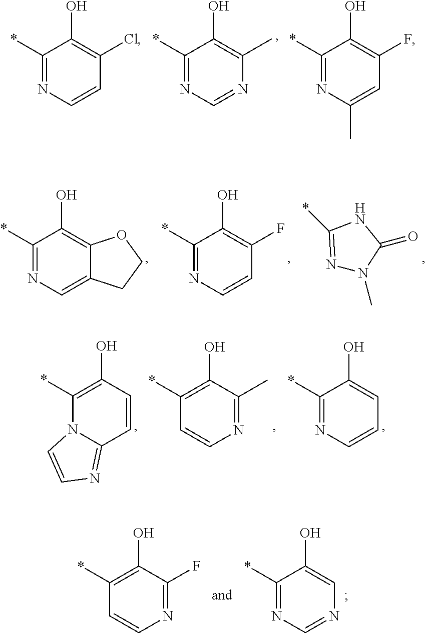

the moiety:

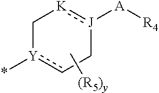

is:

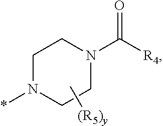

y is 0 or 1;
R₅ is selected from CH₃;
or wherein the moiety is

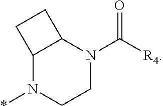

Embodiment 68. A compound of formula (I), or a pharmaceutically acceptable salt thereof, according to any of embodiments 1 to 67, wherein the compound is selected from:

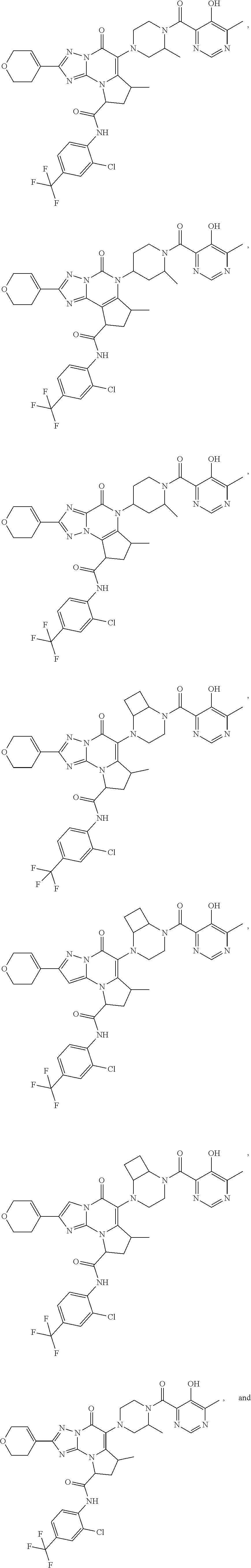

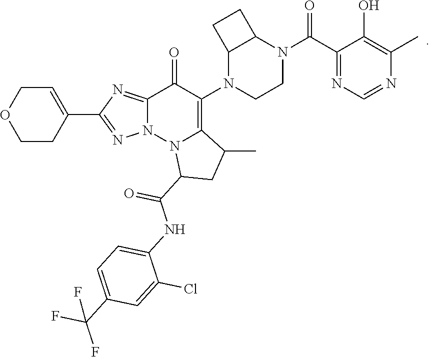 [SMILES: Cc1ncnc(C(=O)N2CCN(c3c4n(n5nc(C6=CCOCC6)nc5c3=O)C(C(=O)Nc3ccc(C(F)(F)F)cc3Cl)CC4C)C3CCC32)c1O]

Embodiment 69. A compound of formula (I), or a pharmaceutically acceptable salt thereof, according to any of Embodiments 1-68, wherein the compound contains a R₄ moiety in non-zwitterionic form.

Embodiment 70. A compound of formula (I), or a pharmaceutically acceptable salt thereof, according to any of Embodiments 1-68, wherein the compound contains a R₄ moiety in zwitterionic form.

Embodiment 71. A compound of formula (I), or a pharmaceutically acceptable salt thereof, according to any of Embodiments 1-68, wherein the compound contains a R₄ moiety which is a mixture of zwitterionic and non-zwitterionic forms.

Embodiment 72. A compound of formula (I), or a pharmaceutically acceptable salt thereof, according to any of embodiments 1-68, wherein the R₄ moiety is shown below, and said R₄ moiety is present in non-zwitterionic form (d) or (e):

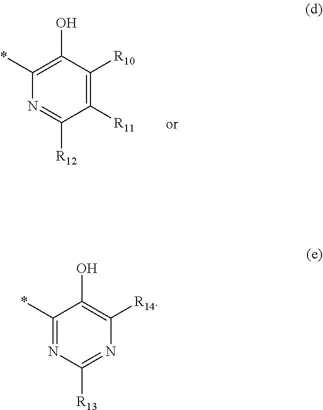

Embodiment 73. A compound of formula (I), or a pharmaceutically acceptable salt thereof, according to any of embodiments 1-68, wherein the R₄ moiety is shown below, and said R₄ moiety is present in a zwitterionic form selected from:

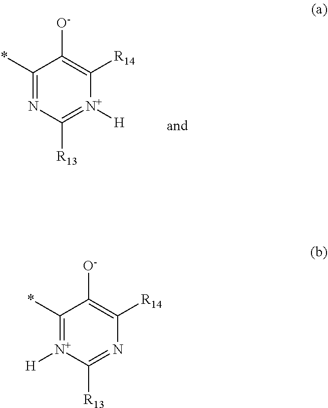

Embodiment 74. A compound of formula (I), or a pharmaceutically acceptable salt thereof, according to any of embodiments 1-68, wherein the R₄ moiety is present as a mixture of zwitterionic forms (a) and (b) according to embodiment 72.

Embodiment 75. A compound of formula (I), or a pharmaceutically acceptable salt thereof, according to any of embodiments 1-74, wherein the R₄ moiety is present as a mixture of:
Non-zwitterionic form (e) and zwitterionic forms (a) or (b),

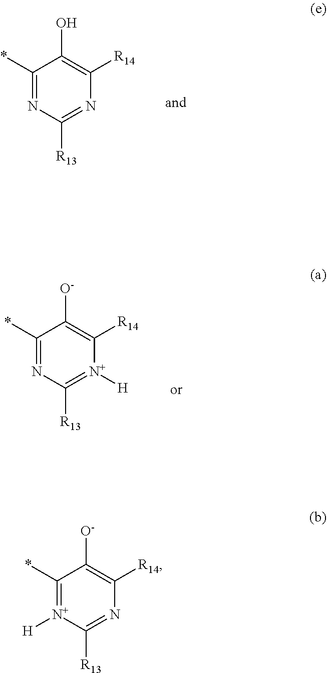

Non-zwitterionic form (e) and zwitterionic forms (a) and (b)

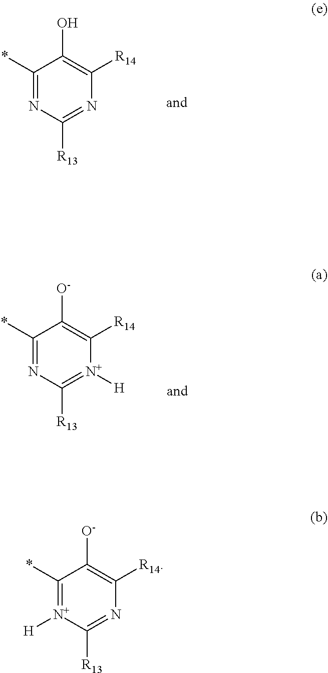

Embodiment 76. A compound of formula (I), or a pharmaceutically acceptable salt thereof, according to any of embodiments 1-74, wherein the R₄ moiety is in zwitterionic form (c):

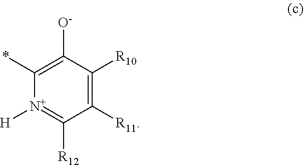

Embodiment 77. A compound of formula (I), or a pharmaceutically acceptable salt thereof, according to any of embodiments 1-74, wherein the R₄ moiety is present as a mixture of both zwitterionic form (c) and non-zwitterionic form (d):

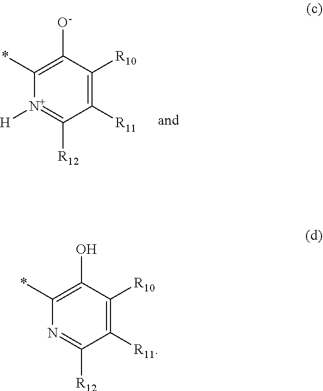

Embodiment 78. A compound of formula (I), according to any of embodiments 1 to 44, 50, 51, 52, 53, 54, 55 or 56, wherein the compound is (7R,9R)—N-(2-chloro-4-(trifluoromethyl)phenyl)-2-(3,6-dihydro-2H-pyran-4-yl)-6-((R)-4-(5-hydroxy-6-methylpyrimidine-4-carbonyl)-3-methylpiperazin-1-yl)-7-methyl-5-oxo-5,7,8,9-tetrahydropyrrolo[1,2-c][1,2,4]triazolo[1,5-a]pyrimidine-9-carboxamide:
in non-zwitterionic form:

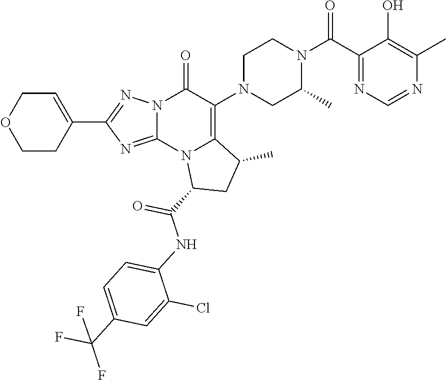 [SMILES: Cc1ncnc(C(=O)N2CCN(c3c4n(c5nc(C6=CCOCC6)nn5c3=O)[C@@H](C(=O)Nc3ccc(C(F)(F)F)cc3Cl)C[C@H]4C)C[C@H]2C)c1O]

or zwitterionic form:

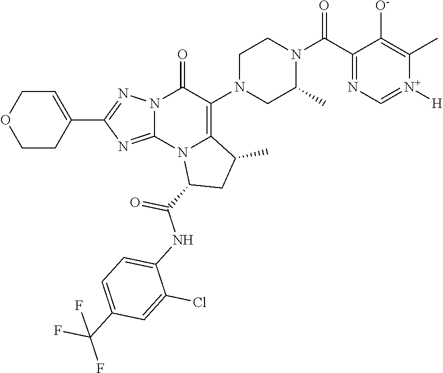 [SMILES: Cc1[nH+]cnc(C(=O)N2CCN(c3c4n(c5nc(C6=CCOCC6)nn5c3=O)[C@@H](C(=O)Nc3ccc(C(F)(F)F)cc3Cl)C[C@H]4C)C[C@H]2C)c1[O-]]

or zwitterionic form:

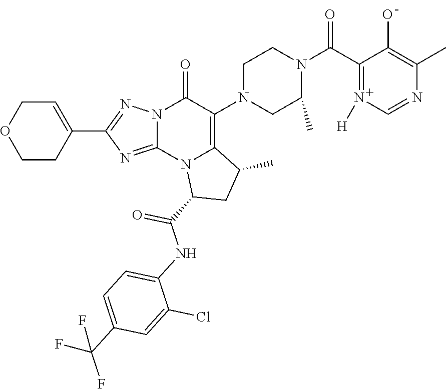

or a mixture of any two or three of said forms.

Embodiment 79. A compound of formula (I), according to any of embodiments 1 to 44, 50, 51, 52, 53, 54, 55 or 57, wherein the compound is (7R,9R)—N-(2-chloro-4-(trifluoromethyl)phenyl)-2-(3,6-dihydro-2H-pyran-4-yl)-6-((1S,6S)-5-(5-hydroxy-6-methylpyrimidine-4-carbonyl)-2,5-diazabicyclo[4.2.0]octan-2-yl)-7-methyl-5-oxo-5,7,8,9-tetrahydropyrrolo[1,2-c][1,2,4]triazolo[1,5-a]pyrimidine-9-carboxamide:
in non-zwitterionic form

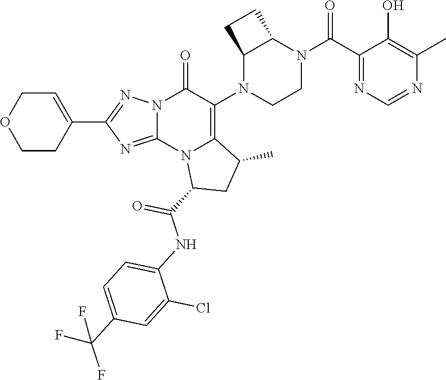 [SMILES: Cc1ncnc(C(=O)N2CCN(c3c4n(c5nc(C6=CCOCC6)nn5c3=O)[C@@H](C(=O)Nc3ccc(C(F)(F)F)cc3Cl)C[C@H]4C)[C@H]3CC[C@@H]32)c1O]

or in zwitterionic form:

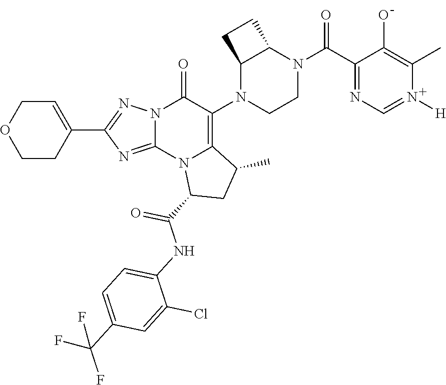

or in zwitterionic form:

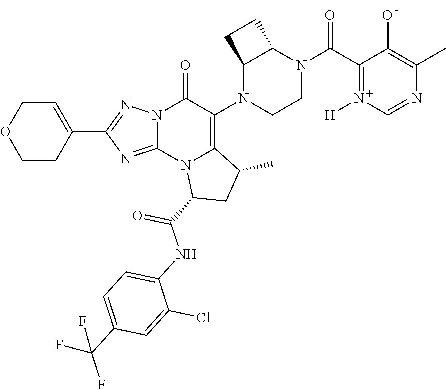

or a mixture of any two or three of said forms.

Embodiment 80. A compound of formula (I), or a pharmaceutically acceptable salt thereof, according to any of embodiments 1-79, in crystalline form.

Embodiment 81. A compound of formula (I) according to any of embodiments 1 to 44, 50, 51, 52, 53, 54, 55 or 56 or 78, wherein the compound is (7R,9R)—N-(2-chloro-4-(trifluoromethyl)phenyl)-2-(3,6-dihydro-2H-pyran-4-yl)-6-((R)-4-(5-hydroxy-6-methylpyrimidine-4-carbonyl)-3-methylpiperazin-1-yl)-7-methyl-5-oxo-5,7,8,9-tetrahydropyrrolo[1,2-c][1,2,4]triazolo[1,5-a]pyrimidine-9-carboxamide, in crystalline form.

Embodiment 82. A compound of formula (I) according to any of embodiments 1 to 44, 50, 51, 52, 53, 54, 55 or 57 or 79, wherein the compound is (7R,9R)—N-(2-chloro-4-(trifluoromethyl)phenyl)-2-(3,6-dihydro-2H-pyran-4-yl)-6-((1S,6S)-5-(5-hydroxy-6-methylpyrimidine-4-carbonyl)-2,5-diazabicyclo[4.2.0]octan-2-yl)-7-methyl-5-oxo-5,7,8,9-tetrahydropyrrolo[1,2-c][1,2,4]triazolo[1,5-a]pyrimidine-9-carboxamide, in crystalline form.

Embodiment 83. A compound of formula (I) according to any of embodiments 80 to 82, wherein the compound is in substantially pure form.

Embodiment 84. A compound of formula (I) according to embodiment 1, wherein the crystalline form
according to embodiment 81 is characterized by a X-ray powder diffraction pattern comprising 4 or more 2θ values selected from the group consisting of 8.6±0.2, 11.2±0.2, 13.0±0.2, 14.9±0.2, 15.5±0.2, 17.9±0.2, 19.4±0.2, 22.0±0.2, 24.3±0.2, 26.0±0.2, 28.2±0.2, 29.1±0.2 and 29.7±0.2 at a temperature of about 22° C., and
according to embodiment 82 is characterized by a X-ray powder diffraction pattern comprising 4 or more 2θ values selected from the group consisting of 5.77±0.2, 6.67±0.2, 10.54±0.2, 12.36±0.2, 12.90±0.2, 13.02±0.2, 14.83±0.2, 15.27±0.2, 15.65±0.2, 15.99±0.2, 17.34±0.2, 18.52±0.2, 19.18±0.2, 20.04±0.2, 21.17±0.2, 21.47±0.2, 21.86±0.2, 22.96±0.2, 23.18±0.2 and 23.9±0.2 at a temperature of about 22° C.

Embodiment 85. A compound of formula (I), according to embodiment 1, wherein the crystalline form
according to embodiment 81 is characterized by a X-ray powder diffraction pattern comprising 5 or more 2θ values selected from the group consisting of 8.6±0.2, 11.2±0.2, 13.0±0.2, 14.9±0.2, 15.5±0.2, 17.9±0.2, 19.4±0.2, 22.0±0.2, 24.3±0.2, 26.0±0.2, 28.2±0.2, 29.1±0.2 and 29.7±0.2 at a temperature of about 22° C., and
according to embodiment 82 is characterized by a X-ray powder diffraction pattern comprising 5 or more 2θ values selected from the group consisting of 5.77±0.2, 6.67±0.2, 10.54±0.2, 12.36±0.2, 12.90±0.2, 13.02±0.2, 14.83±0.2, 15.27±0.2, 15.65±0.2, 15.99±0.2, 17.34±0.2, 18.52±0.2, 19.18±0.2, 20.04±0.2, 21.17±0.2, 21.47±0.2, 21.86±0.2, 22.96±0.2, 23.18±0.2 and 23.9±0.2 at a temperature of about 22° C.

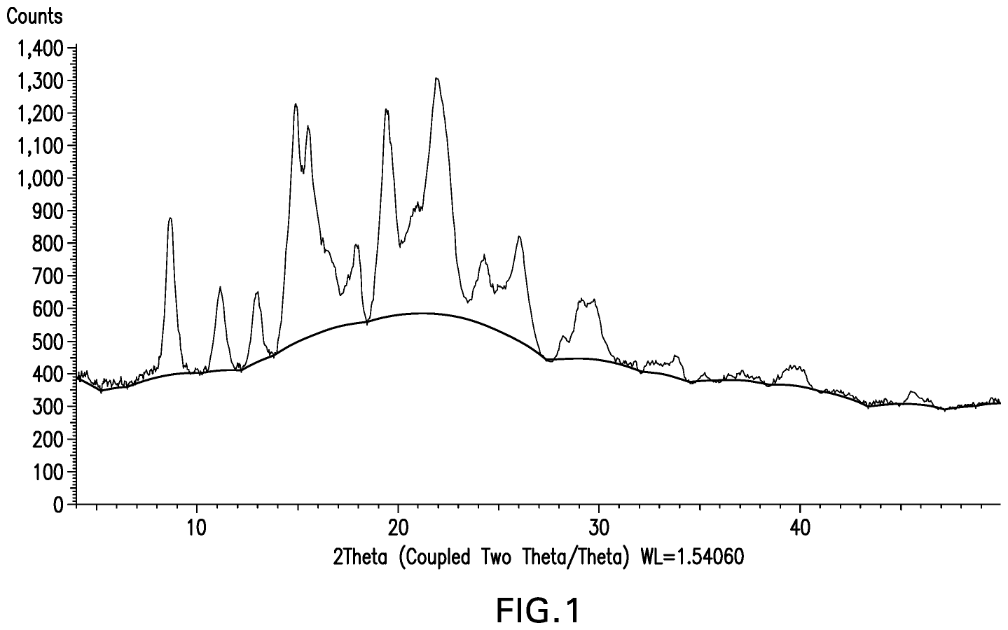

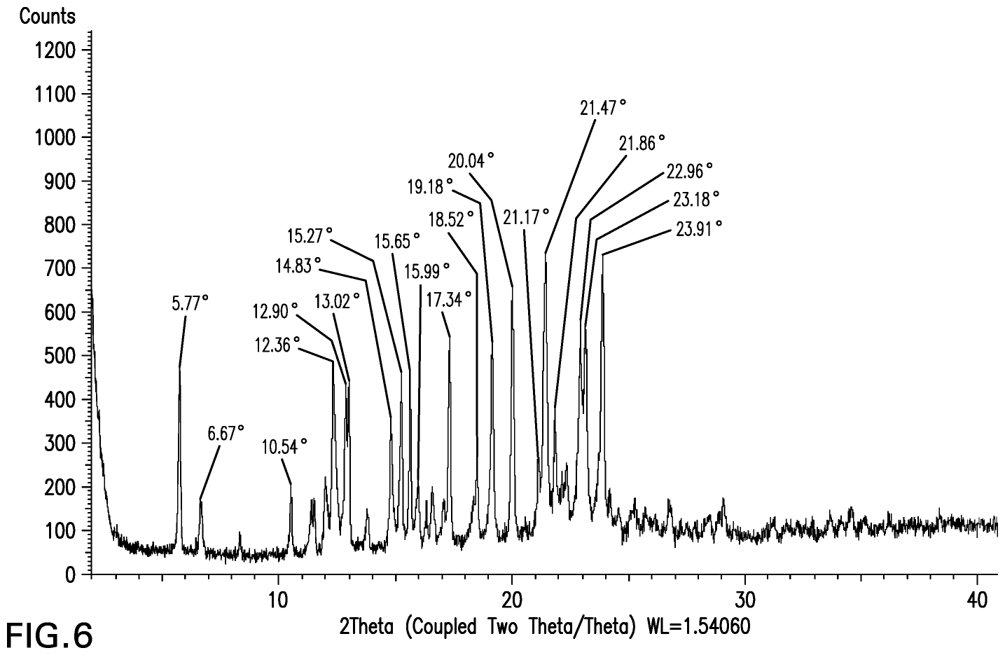

Embodiment 86. A compound of formula (I) according to embodiment 1, wherein the crystalline form
according to embodiment 81 is characterized by a X-ray diffraction pattern substantially the same as the X-ray powder diffraction pattern shown in FIG. 1
according to embodiment 82 is characterized by a X-ray diffraction pattern substantially the same as the X-ray powder diffraction pattern shown in FIG. 6

Embodiment 87. A compound of formula (I), or a pharmaceutically acceptable salt thereof, according to any of embodiments 1-79 and 83, wherein the compound is in amorphous form.

Embodiment 88. A compound of formula (I), or a pharmaceutically acceptable salt thereof, according to any of embodiments 1-83, wherein the compound is a sodium salt.

In one embodiment, there is provided a compound of formula (I) or a pharmaceutically acceptable salt thereof, wherein when R₁ is a ring, then:
each R₁ ring atom adjacent to the R₁ ring atom to which said R₁ ring is joined to the remainder of the molecule, is independently unsubstituted or substituted by halo only, in particular, independently unsubstituted or substituted with one F substituent, and
preferably, said R₁ ring is linked to the remainder of the molecule via a R₁ ring nitrogen atom, or a R₁ ring carbon atom which is double-bonded to an adjacent R₁ ring atom.

In particular, R₁ is:
cycloalkenyl, wherein said cycloalkenyl is a partially unsaturated monocyclic ring containing 5 or 6 ring carbon atoms, and said cycloalkenyl is unsubstituted or substituted by 1, 2, 3 or 4, preferably 1 or 2, R₃₃, wherein R₃₃ is halo, and wherein said cycloalkenyl or halo-substituted cycloalkenyl is substituted by 0, 1 or 2 R₁₅ substituents,
or R₁ is heterocyclyl, wherein said heterocyclyl is a 5 or 6 membered fully saturated or partially unsaturated group comprising ring carbon atoms and 1 or 2 ring heteroatoms independently selected from N, NH, O and S, and wherein said heterocyclyl is unbridged or bridged, and said bridge is 1 or 2 carbon atoms, wherein said heterocyclyl is unsubstituted or substituted by 1, 2, 3 or 4, preferably 1 or 2, R₃₃, wherein R₃₃ is halo, and wherein said heterocyclyl or halo-substituted heterocyclyl is substituted by 0, 1 or 2 substituents independently selected from R₁₅, R₁₆, R₁₇, R₁₈, R₁₉, R₂₀, R₂₂ and R₂₃,
or R₁ is heteroaryl, wherein said heteroaryl is a 5 or 6 membered fully unsaturated monocyclic group comprising ring carbon atoms and 1, 2, 3 or 4 ring heteroatoms independently selected from N, O and S, preferably 1 or 2 ring heteroatoms, wherein the total number of ring S atoms does not exceed 1, and the total number of ring O atoms does not exceed 1, wherein said heteroaryl is unsubstituted or substituted by 1, 2 or 3 substituents independently selected from R₂, and R₃₀, wherein R₂₁ and R₃₀ are independently selected from halo and (C₁-C₄)alkyl, wherein said (C₁-C₄)alkyl is unsubstituted or substituted by 1, 2 or 3 halo,
and each R₁₅, R₁₆, R₁₇, R₁₈, R₁₉, R₂₀, R₂₂ and R₂₃ is independently selected from:
   halo
   (C₁-C₄)alkyl-O— unsubstituted or substituted by 1, 2 or 3 halo;
   (C₁-C₄)alkyl unsubstituted or substituted by OH, —O—(C₁-C₂)alkyl or 1, 2 or 3 halo,
   HOC(O)—(CH₂)ₙ—,
   H₃C—C(O)(CH₂)ₙ—,
   (C₁-C₄)alkyl-O—C(O)(CH₂)ₙ,
   ═O
   azetidinyl or pyrrolidinyl, wherein said azetidinyl and pyrrolidinyl are linked to the rest of the molecule via the N atom, and are each unsubstituted or substituted by 1 or 2 F,
   R₂₅(R₂₄)N—, wherein R₂₄ is H or (C₁-C₄)alkyl unsubstituted or substituted by 1, 2 or 3 halo, R₂₅ is H or (C₁-C₄)alkyl unsubstituted or substituted by 1, 2 or 3 halo,
   OH
wherein n is 0, 1 or 2,

More particularly, R₁ is:
cycloalkenyl, wherein said cycloalkenyl is a partially unsaturated monocyclic ring containing 5 or 6 ring carbon atoms, and said cycloalkenyl is unsubstituted or substituted by 1 or 2 R₃₃, wherein R₃₃ is halo, preferably F, and wherein said cycloalkenyl or halo-substituted cycloalkenyl is substituted by 0 or 1 R₁₅ substituents, preferably 1 substituent, wherein R₁₅ is selected from:
   h) (C₁-C₂)alkyl-O— unsubstituted or substituted by 1, 2 or 3 halo;
   i) (C₁-C₂)alkyl unsubstituted or substituted by 1, 2 or 3 halo,
   j) HOC(O)—(CH₂)ₙ—,
   k) H₃C—C(O)(CH₂)ₙ—,
   l) H₃C—O—C(O)(CH₂)ₙ,
   m)═O, and
   n) R₂₅(R₂₄)N—, H, wherein R₂₄ is H or (C₁-C₂)alkyl unsubstituted or substituted by 1, 2 or 3 halo, R₂₅ is H or (C₁-C₂)alkyl unsubstituted or substituted by 1, 2 or 3 halo,
n is 0 or 1,
wherein
   the R₁₅ substituent a) to g) of said cycloalkenyl or halo-substituted cycloalkenyl is not present on the ring atoms adjacent to the ring atom to which the cycloalkenyl or halo-substituted cycloalkenyl is joined to the remainder of the molecule, and preferably, said cycloalkenyl or halo-substituted cycloalkenyl is a 6 membered ring, with 1 R₁₅ substituent in the ring para position relative to the remainder of the molecule; and
   said cycloalkenyl or halo-substituted cycloalkenyl is linked to the remainder of the compound via a R₁ ring carbon atom which is double bonded to an adjacent R₁ ring carbon atom;
or R₁ is heterocyclyl, wherein said heterocyclyl is a 5 or 6 membered fully saturated or partially unsaturated group comprising ring carbon atoms and 1 or 2 ring heteroatoms independently selected from N, NH, 0 and S, and wherein said heterocyclyl is unbridged or bridged, and said bridge is 1 or 2 carbon atoms, wherein said heterocyclyl is unsubstituted or substituted by 1 or 2 R₃₃, wherein R₃₃ halo, is preferably F, and wherein said heterocyclyl or halo-substituted heterocyclyl is substituted by 0 or 1 substituents independently selected from R₁₅, R₁₆, R₁₇, R₁₈, R₁₉, R₂₀, R₂₂ and R₂₃, wherein said R₁₅, R₁₆, R₁₇, R₁₈, R₁₉, R₂₀, R₂₂ and R₂₃ are independently selected from:
   i) (C₁-C₄)alkyl-O— unsubstituted or substituted by 1, 2 or 3 halo;
   j) (C₁-C₄)alkyl unsubstituted or substituted by OH, —O—(C₁-C₂)alkyl or 1, 2 or 3 halo,
   k) HOC(O)—(CH₂)ₙ—,
   l) H₃C—C(O)(CH₂)ₙ—,
   m) H₃C—O—C(O)(CH₂)ₙ,
   n)=0
   o) R₂₅(R₂₄)N—, wherein R₂₄ is H, (C₁-C₂)alkyl unsubstituted or substituted by 1, 2 or 3 halo, R₂₅ is H, (C₁-C₂)alkyl unsubstituted or substituted by 1, 2 or 3 halo,
   p) OH
wherein n is 0 or 1,
and wherein:
   substituent a) to h) of said heterocyclyl or halo-substituted heterocyclyl is not present on the ring atoms adjacent to the ring atom to which the heterocyclyl or halo-substituted heterocyclyl is joined to the remainder of the molecule, and preferably, when said heterocyclyl or halo-substituted heterocyclyl is a 6 membered ring, it has 0 or 1 substituent selected from a) to h) in the meta or para position, preferably para, relative to the remainder of the molecule; and
   said heterocyclyl is linked to the remainder of the compound via a R₁ ring nitrogen atom, or a R₁ ring carbon atom which is double bonded to an adjacent ring atom;
or R₁ is heteroaryl, wherein said heteroaryl is a 5 or 6 membered fully unsaturated monocyclic group comprising ring carbon atoms and 1 or 2 ring heteroatoms independently selected from N, O and S, preferably N, wherein the total number of ring S atoms does not exceed 1, and the total number of ring O atoms does not exceed 1, wherein said heteroaryl is unsubstituted or substituted by 1 or 2 substituents independently selected from R₂, and R₃₀, wherein R₂₁ and R₃₀ are independently selected from (C₁-C₂)alkyl, and said (C₁-C₂)alkyl is unsubstituted or substituted by 1, 2 or 3 halo, and wherein preferably, said alkyl or halo-alkyl substituent is not present on the R₁ ring atoms adjacent to the R₁ ring atom to which the heteroaryl is joined to the remainder of the molecule, and more preferably, when heteroaryl is a 6-membered ring, said alkyl or halo-alkyl substituent is in the ring para position relative to the rest of the molecule.

More particularly, R₁ is selected from:

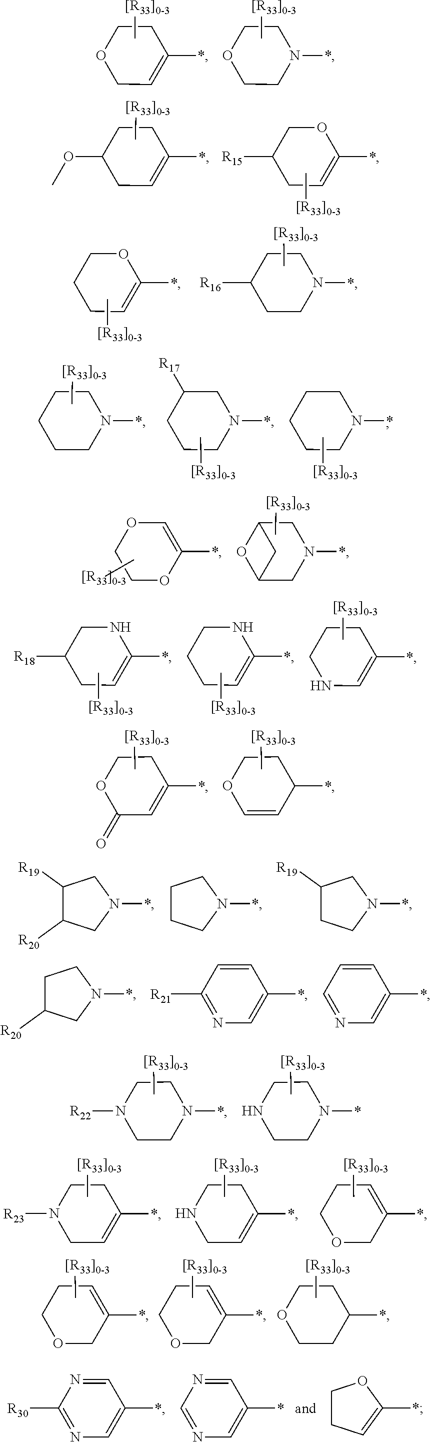

R₃₃ is F;
R₁₅ is halo, azetidinyl or pyrrolidinyl, wherein said azetidinyl and pyrrolidinyl are linked to the rest of the molecule via the N atom, and are unsubstituted or substituted by 1 or 2 F;
R₁₆ is R₂₅(R₂₄)N—, wherein R₂₄ is H or (C₁-C₂)alkyl, R₂₅ is H or (C₁-C₂)alkyl unsubstituted or substituted by 1, 2 or 3 halo, in particular F;
R₁₇ is halo
R₁₈ is halo;
R₁₉ is halo;
R₂₀ is halo;
R₂₁ is (C₁-C₂)alkyl;
R₂₂ and R₂₃ are each independently selected from:
   (C₁-C₄)alkyl unsubstituted or substituted by 1, 2 or 3 halo,
   HOC(O)—(CH₂)ₙ—,
   H₃C—C(O)(CH₂)ₙ—,
   (H₃C)₃C—O—C(O)(CH₂)ₙ—;
   wherein n is 0, 1 or 2;
and
R₃₀ is CH₃.

In a particular embodiment, R₁ is selected from:

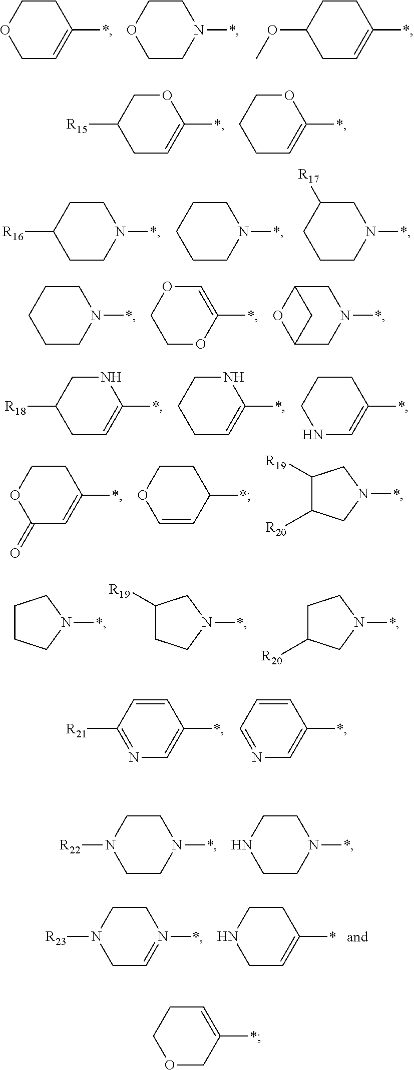

R₁₅ s F;
R₁₆ is R₂₅(R₂₄)N—;
R₁₇ is F;
R₁₈ is F;
R₁₉ is F;
R₂₀ is F;
R₂₁ is CH₃;
R₂₂ is CF₃, CHF₂CH₂, HOC(O)—CH₂—, H₃C—C(O)—, (H₃C)₃C—O—C(O)—;
R₂₃ is CF₃, CHF₂CH₂—, (H₃C)₃C—O—C(O)—;
R₂₄ is CH₃; and
R₂₅ is CHF₂CH₂—.

In another embodiment, R₁ is selected from:

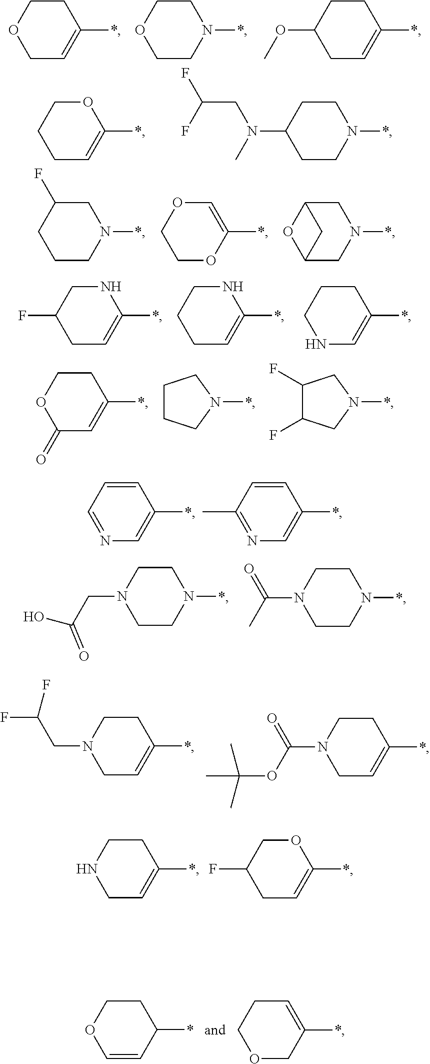

especially,

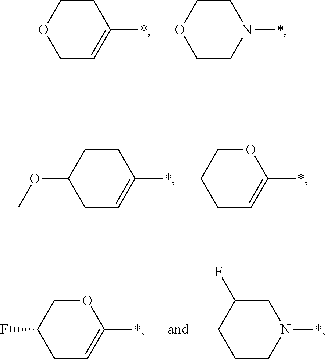

in particular

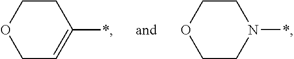

more particularly

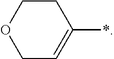

In another embodiment, there is provided a compound of formula (I) or a pharmaceutically acceptable salt thereof, wherein R₂ is the moiety:

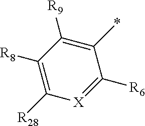

R₆ is selected from:
   H,
   halo,
   (C₁-C₄)alkyl unsubstituted or substituted by 1, 2 or 3 halo,
   (C₃-C₅)cycloalkyl unsubstituted or substituted by 1, 2 or 3 halo,
   —O—(C₁-C₄)alkyl unsubstituted or substituted by 1, 2 or 3 halo,
   OH, and
   CN;
R₈ is selected from H, halo, and (C₁-C₄)alkyl unsubstituted or substituted by 1, 2 or 3 halo,
R₉ is selected from H, O—CH₃, OH, CN, CH₃ and halo;
R₂₈ is selected from:
   SF₅,
   H,
   —C(O)H,
   halo,
   (C₁-C₄)alkyl unsubstituted or substituted by 1, 2 or 3 halo,
   (C₁-C₄)alkynyl,
   (C₁-C₄)alkenyl,
   (C₃-C₅)cycloalkyl unsubstituted or substituted by 1, 2 or 3 halo, and
   OCF₃;
and X is selected from C—R₇ and N, wherein R₇ is H or halo.

In particular, R₂ is the moiety:

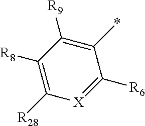

wherein
R₆ is selected from H, halo, (C₁-C₄)alkyl unsubstituted or substituted by 1, 2 or 3 halo;
R₈ is selected from H, halo, (C₁-C₄)alkyl unsubstituted or substituted by 1, 2 or 3 halo;
R₉ is selected from H, O—CH₃, OH, CN, CH₃ and halo;
R₂₈ is selected from SF₅, halo, C(O)H and (C₁-C₄)alkyl unsubstituted or substituted by 1, 2 or 3 halo;
X is selected from C—R₇ and N; and
R₇ is selected from H and halo.

More particularly, R₂ is the moiety:

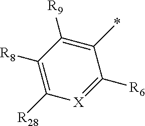

R₆ is selected from H, Cl, CH₃, F and Br;
R₈ is selected from H, Cl, F and CF₃;
R₉ is selected from H, CH₃ and C;
R₂₈ is selected from CF₃, CF₂H, —CH₂CH₃, Cl, SF₅, Br and —C(O)H;
X is selected from C—R₇ and N; and
R₇ is selected from H and F.

In one embodiment, the moiety:

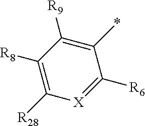

is selected from:

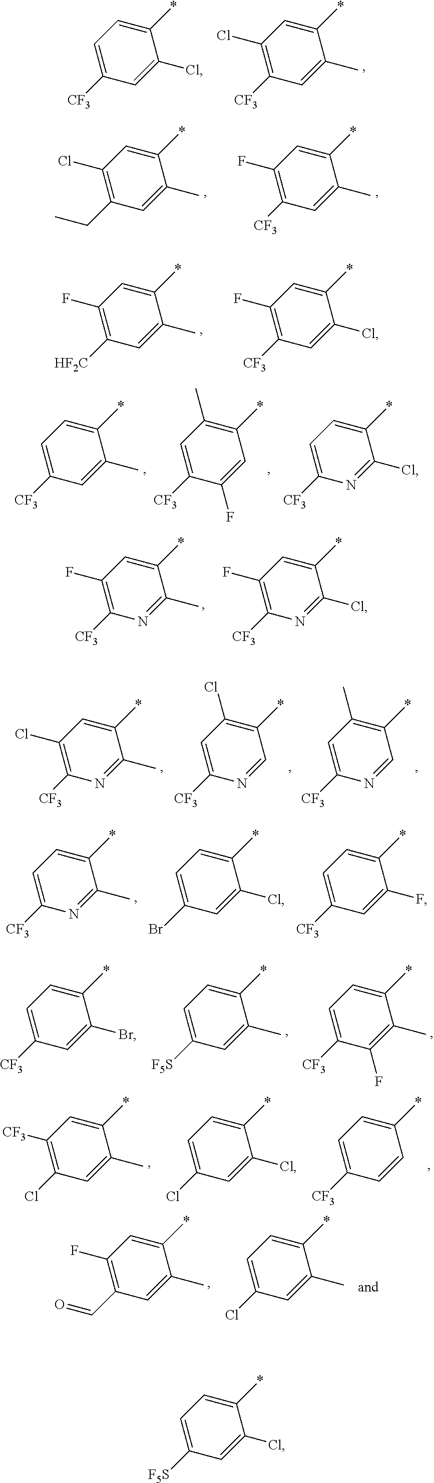

in particular

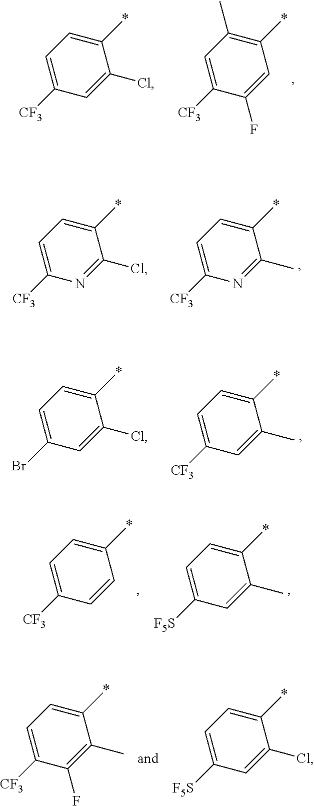

more particularly

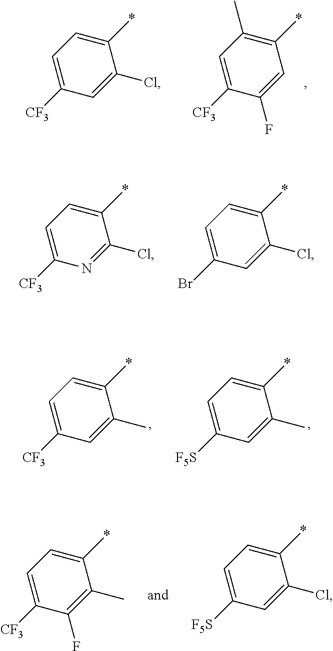

especially

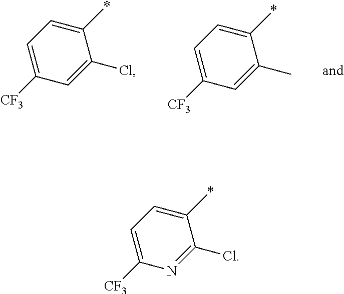 [SMILES: *c1ccc(C(F)(F)F)cc1Cl ; *c1ccc(C(F)(F)F)cc1C ; *c1ccc(C(F)(F)F)nc1Cl]

In another embodiment, there is provided a compound of formula (I) or a pharmaceutically acceptable salt thereof, wherein x is 0 or 1, particular 1.

In another embodiment, there is provided a compound of formula (I) or a pharmaceutically acceptable salt thereof, wherein R₃ is (C₁-C₄)alkyl unsubstituted or substituted by 1, 2 or 3 substituents independently selected from halo and OH, in particular R₃ is (C₁-C₂)alkyl unsubstituted or substituted by 1, 2 or 3 substituents independently selected from halo and OH, preferably R₃ is —CH₂CH₃ or CH₃, more preferably CH₃.

In another embodiment, R₃ is in the position shown in formula 1i:

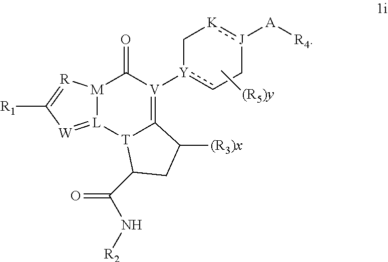

In another embodiment, there is provided a compound of formula (I) or a pharmaceutically acceptable salt thereof, wherein Y is N and Y [symbol] is Y linked by a single bond. In particular, wherein K [symbol] is K linked by a single bond, and K is selected from —CH₂—, —CH₂CH₂—, —NH— and a bond (to form a 5-membered ring:

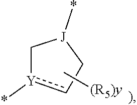

J is N, and A is a linker selected from —C(O)—, —S(O)—, —S(O)₂—, and

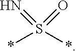

More particularly, K [symbol] is K linked by a single bond K is —CH₂—, J is N, and A is a linker selected from —C(O)—, —S(O)—, —S(O)₂—, and

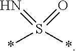

In one embodiment, A is a linker selected from —C(O)— and —S(O)₂—, preferably —C(O)—.

In one embodiment, there is provided a compound of formula (I), or a pharmaceutically acceptable salt thereof, as described herein, wherein the compound of formula (I) has the stereochemistry shown in formula (I′):

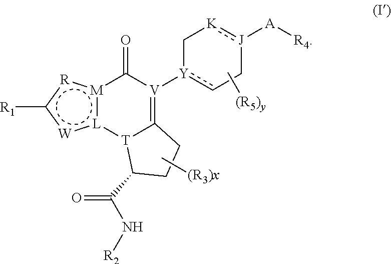

When the compound of formula (I) is a compound of formula (I″):

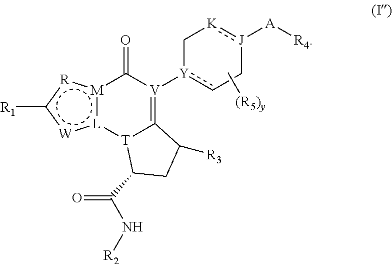

said compound, in particular, has the stereochemistry of formula (I′″):

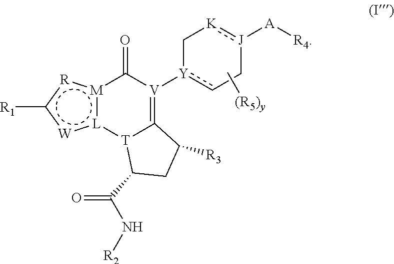

In another embodiment, there is provided a compound of formula (I) or a pharmaceutically acceptable salt thereof, wherein R₅ is independently selected from:
—(C₁-C₄)alkyl, preferably methyl,
and wherein two R₅ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S,
when K [symbol] J is a carbon-nitrogen single bond, a R₅ substituent on K and on the adjacent carbon atom may join to form ring C:

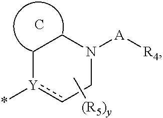

wherein ring C is a fused (C₃-C₆)cycloalkyl ring, in particular a fused cyclobutyl ring, a fused (C₃-C₆)heterocyclyl ring or a fused phenyl ring, wherein said fused (C₃-C₆)heterocyclyl ring contains ring carbon atoms and one ring heteroatom selected from O, N and S,
   and wherein when ring C is a fused (C₃-C₆)cycloalkyl ring, in particular fused cyclobutyl ring, said fused (C₃-C₆)cycloalkyl ring is unsubstituted or substituted with 1 or 2 R₄₀ groups, wherein said R₄₀ is selected from:
      (C₁-C₂)alkyl, wherein each (C₁-C₂)alkyl is independently unsubstituted or substituted by OH or 1, 2 or 3 halo,
      halo, in particular F,
      or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
      or wherein two R₄₀ substituents on adjacent carbon atoms join together with the carbon atoms to which they are attached, to form a fused cyclopropyl ring;
   and wherein when K is —CH₂— and J is N, two R₅ substituents may join to form a (C₁-C₃)alkylene bridge or a heteroalkylene bridge, wherein said heteroalkylene bridge is one heteroatom selected from N and O, or is —CH₂—O—CH₂—.

In particular, R₅ is independently selected from:
—(C₁-C₄)alkyl, preferably methyl,
when K [symbol] J is a carbon-nitrogen single bond, a R₅ substituent on K and on the adjacent carbon atom may join to form ring C:

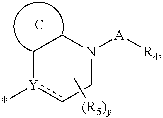

wherein ring C is a fused (C₃-C₆)cycloalkyl ring, in particular a fused cyclobutyl ring, or a fused (C₃-C₆)heterocyclyl ring, wherein said fused (C₃-C₆)heterocyclyl ring contains ring carbon atoms and one ring heteroatom selected from O, N and S,
   and wherein when ring C is a fused (C₃-C₆)cycloalkyl ring, in particular fused cyclobutyl ring, said fused (C₃-C₆)cycloalkyl ring is unsubstituted or substituted with 1 or 2 R₄₀ groups, wherein said R₄₀ is selected from:
      (C₁-C₂)alkyl, wherein each (C₁-C₂)alkyl is independently unsubstituted or substituted by OH or 1, 2 or 3 halo,
      halo, in particular F,
      or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
      or wherein two R₄₀ substituents on adjacent carbon atoms join together with the carbon atoms to which they are attached, to form a fused cyclopropyl ring;
and wherein when K is —CH₂— and J is N, two R₅ substituents may join to form a (C₁-C₃)alkylene bridge or a heteroalkylene bridge, wherein said heteroalkylene bridge is one heteroatom selected from N and O, or is —CH₂—O—CH₂—.

More particularly, R₅ is independently selected from:
—(C₁-C₂)alkyl, preferably methyl, and
when K [symbol] J is a carbon-nitrogen single bond, a R₅ substituent on K and on the adjacent carbon atom may join to form ring C:

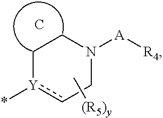

wherein ring C is a fused (C₃-C₄)cycloalkyl ring, in particular a fused cyclobutyl ring, and said fused (C₃-C₄)cycloalkyl ring, in particular fused cyclobutyl ring, is unsubstituted or substituted with 1 or 2 R₄₀ groups as described in embodiment 28.

In one embodiment, y is 0, 1, 2 or 3, preferably 0, 1, or 2.

In a preferred embodiment, R₅ is independently selected from:
CH₃, and y is 1 or 2, and
when K [symbol] J is a carbon-nitrogen single bond, a R₅ substituent on K and on the adjacent carbon atom may join to form ring C:

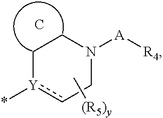

wherein ring C is a fused cyclobutyl ring.

In another embodiment, the compound of formula (I) includes the moiety:

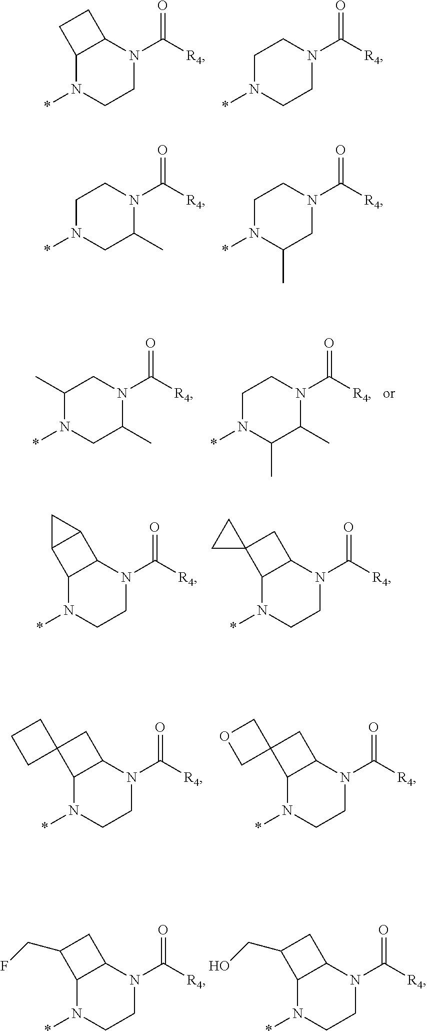

in particular
A:

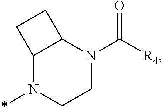

more particularly

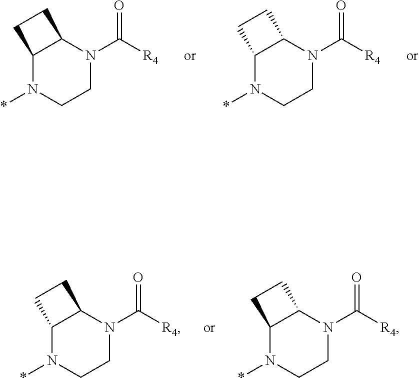

or B:

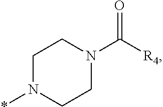

or C:

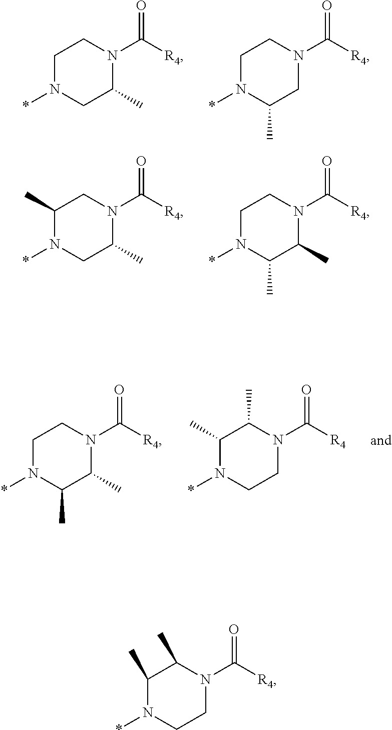

or wherein the linker-C(O)— is replaced by the alternative linkers —S(O)—, —S(O)₂—, and

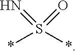

as defined in claim 1.

In another embodiment, there is provided a compound of formula (I) or a pharmaceutically acceptable salt thereof, wherein R₄ is selected from:
—(C₁-C₄)alkyl;
-heteroaryl1, wherein said heteroaryl1 is a 5 or 6 membered, fully unsaturated monocyclic ring comprising ring carbon atoms and 1, 2, 3 or 4 ring heteroatoms independently selected from N, O and S, and the total number of ring S atoms does not exceed 1, and the total number of ring O atoms does not exceed 1;
-heteroaryl2, wherein said heteroaryl2 is a 9 or 10 membered fused bicyclic ring comprising ring carbon atoms and 1, 2, 3 or 4 ring heteroatoms independently selected from N, O and S, and wherein both rings are fully unsaturated, or one ring is fully unsaturated and the other is saturated or partially unsaturated, and wherein the heteroatoms may be in one or both rings, and the total number of ring S atoms does not exceed 1, and the total number of ring O atoms does not exceed 1, and in particular, the ring which is linked to the rest of the molecule via linker -A- is fully unsaturated;
-phenyl;
wherein heteroaryl1, heteroaryl2 and phenyl are each substituted by 1, 2 or 3 substituents independently selected from R₁₀, R₁₁, R₁₂, R₁₃ and R₁₄, wherein each R₁₀, R₁₁, R₁₂, R₁₃ and R₁₄ is independently selected from:
   H,
   halo,
   (C₁-C₄)alkyl unsubstituted or substituted by 1, 2 or 3 halo substituents,
   (C₁-C₂)alkyl substituted by —O—(C₁-C₂)alkyl or OH,
   —S—(C₁-C₃)alkyl,
   —O—(C₁-C₄)alkyl unsubstituted or substituted by 1, 2 or 3 halo substituents,
   OH,
   (C₃-C₅)cycloalkyl, wherein said (C₃-C₅)cycloalkyl is unsubstituted or substituted by 1 or 2 halo,
   —O—(C₃-C₅)cycloalkyl,
   —NR₃₄R₃₅ wherein R₃₄ and R₃₅ are independently selected from:
      H,
      (C₁-C₄)alkyl, wherein said (C₁-C₄)alkyl is unsubstituted or substituted by OH or —O(C₁-C₂)alkyl,
      and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
   CN,
   —(C₂-C₄)alkenyl,
   —(C₂-C₄)alkynyl,
   ═O
   —C(O)H, and
   —C(O)(C₁-C₄)alkyl;
with the proviso that one OH substituent is present on heteroaryl1, heteroaryl2 and phenyl, and the remaining R₁₀, R₁₁, R₁₂, R₁₃ and R₁₄ are as defined herein, and in particular with the proviso that:
one OH substituent is present on heteroaryl1, heteroaryl2 and phenyl, and said OH is in the ortho position of the R₄ ring, relative to the position linking R₄ to linker -A-, or
one ═O substituent is present on heteroaryl1 and heteroaryl2, and the remaining R₁₀, R₁₁, R₁₂, R₁₃ and R₁₄ are defined as herein.

In a particular embodiment, R₄ is selected from:
—(C₁-C₄)alkyl, in particular —CH₃;
-heteroaryl1; and
-heteroaryl2;
-phenyl;
wherein heteroaryl1, heteroaryl2 and phenyl are each substituted by 1, 2 or 3 substituents independently selected from R₁₀, R₁₁, R₁₂, R₁₃ and R₁₄, wherein each R₁₀, R₁₁, R₁₂, R₁₃ and R₁₄ is independently selected from: OH, ═O, H, F, Cl and CH₃, with the proviso that:
   one OH substituent is present on heteroaryl1, heteroaryl2 and phenyl, and said OH is in the ortho position of the R₄ ring, relative to the position linking R₄ to linker -A-, and the remaining substituents are selected from H, F, Cl, and CH₃,
or
   one ═O substituent on said is present on heteroaryl1 and heteroaryl2, and the remaining substituents are selected from H, F, Cl, and CH₃.

More particularly, R₄ is selected from CH₃, heteroaryl1 and heteroaryl2, and the substituents are as defined above. In particular, said heteraryl1 comprises ring carbon atoms and one or two nitrogen atoms only. Preferably, heteroaryl1 is pyridyl or pyrimidinyl.

In one embodiment, R₄ is selected from:
—(C₁-C₄)alkyl, in particular CH₃;

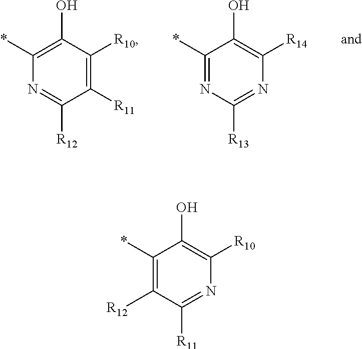

wherein
R₁₀, R₁₁, R₁₂, R₁₃ and R₁₄ are independently selected from:
   H,
   halo,
   (C₁-C₄)alkyl unsubstituted or substituted by 1, 2 or 3 halo substituents,
   (C₁-C₂)alkyl substituted by —O—(C₁-C₂)alkyl or OH,
   —S—(C₁-C₃)alkyl,
   —O—(C₁-C₄)alkyl unsubstituted or substituted by 1, 2 or 3 halo substituents,
   (C₃-C₅)cycloalkyl, wherein said (C₃-C₅)cycloalkyl is unsubstituted or substituted by 1 or 2 halo,
   —O—(C₃-C₅)cycloalkyl,
   —NR₃₄R₃₅ wherein R₃₄ and R₃₅ are independently selected from:
      H,
      (C₁-C₄)alkyl, wherein said (C₁-C₄)alkyl is unsubstituted or substituted by OH or —O(C₁-C₂)alkyl,
      and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
   CN,
   —(C₂-C₄)alkenyl,
   —(C₂-C₄)alkynyl,
   —C(O)H, and
   —C(O)(C₁-C₄)alkyl.

Preferably, R₄ is selected from:

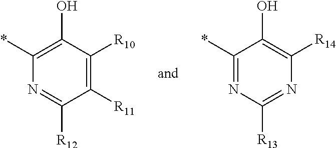

wherein
R₁₀ is selected from H, halo, (C₁-C₂)alkyl unsubstituted or substituted by 1, 2 or 3 halo substituents, —O—(C₁-C₂)alkyl unsubstituted or substituted by 1, 2 or 3 halo substituents;
R₁₁ is selected from H, halo, (C₁-C₂)alkyl unsubstituted or substituted by 1, 2 or 3 halo substituents;
R₁₂ is selected from H, halo, (C₁-C₂)alkyl unsubstituted or substituted by 1, 2 or 3 halo substituents;
R₁₃ is selected from H, —S—CH₃, halo, (C₁-C₂)alkyl unsubstituted or substituted by 1, 2 or 3 halo substituents; and
R₁₄ is selected from H, halo, (C₁-C₂)alkyl unsubstituted or substituted by 1, 2 or 3 halo substituents, O—(C₁-C₂)alkyl unsubstituted or substituted by 1, 2 or 3 halo substituents, and cyclopropyl,

In particular, R₄ is selected from:

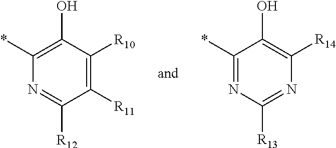

wherein
R₁₀ is selected from H, F, Cl, CH₃ and OCF₃;
R₁₁ is selected from H, F, Cl and CH₃;
R₁₂ is selected from H, F, Cl and CH₃;
R₁₃ is selected from H, F, Cl, —S—CH₃ and CH₃; and
R₁₄ is selected from H, F, Cl, CH₃, —CH₂CH₃, cyclopropyl, —OCHF₂, OCF₃.

In particular, at least one substituent of R₁₀, R₁₁ and R₁₂, is H.

In particular, at least one substituent of R₁₃ and R₁₄, is H.

In another embodiment, R₄ is selected from:
—CH₃,

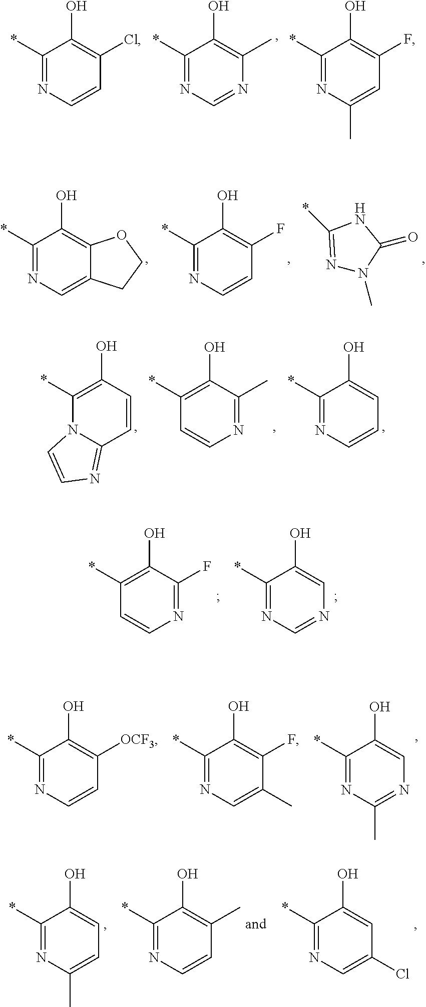

in particular
—CH₃,

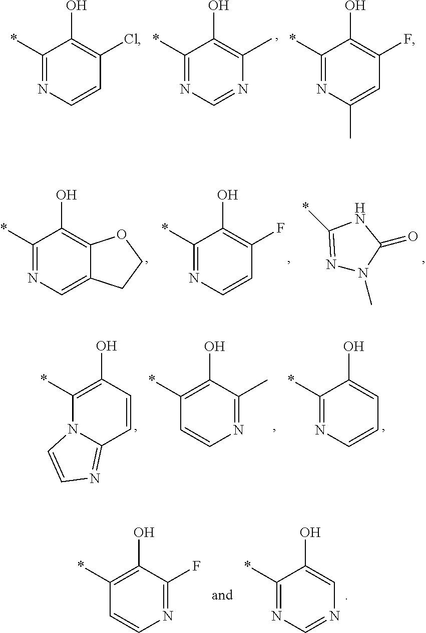

Preferably, R₄ is selected from:

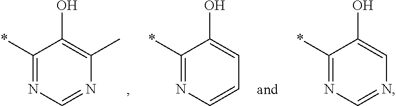

in particular

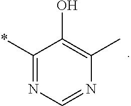 [SMILES: *c1ncnc(C)c1O]

In other embodiments, there is provided a compound of formula (I), or a pharmaceutically acceptable salt thereof, wherein formula (I) is
formula 1a:

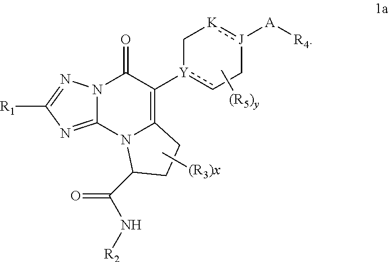

In particular, 1a is

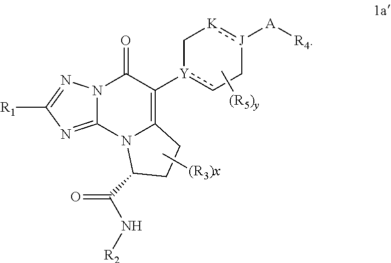

or formula 1b:

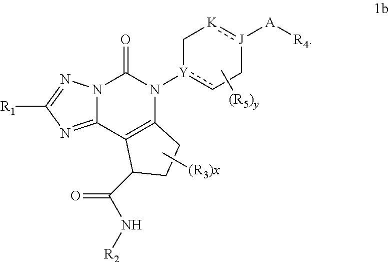

In particular, 1b is

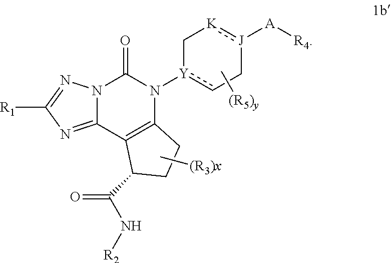

or formula 1c:

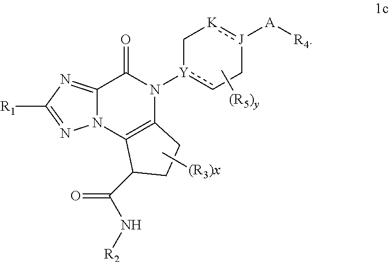

In particular, 1c is

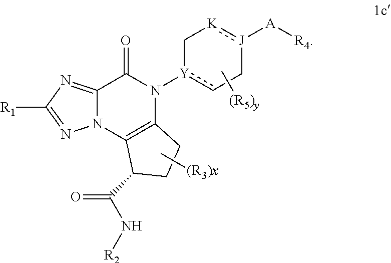

or formula 1d:

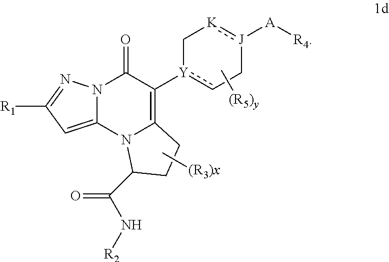

In particular, 1d is 1d′:

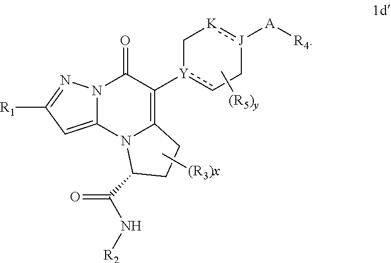

or formula 1e:

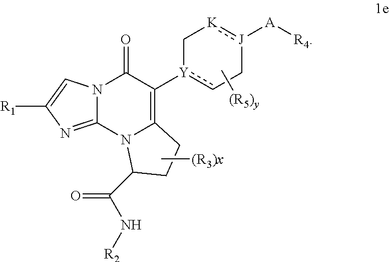

In particular, 1e is 1e′:

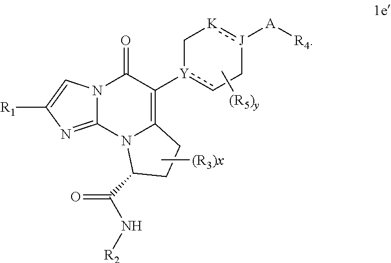

or formula 1f:

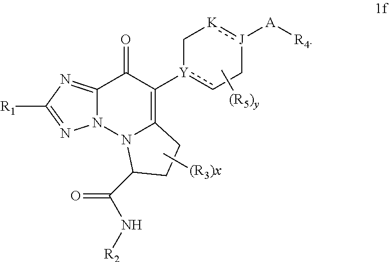

In particular, 1f is 1f:

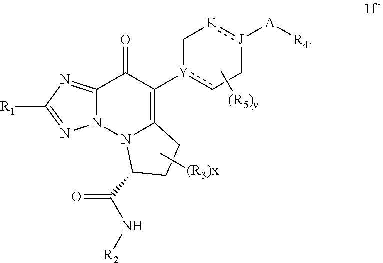

or formula 1g:

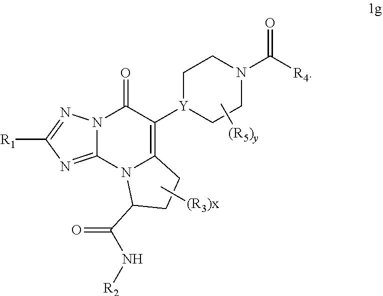

In particular, 1g is 1g′:

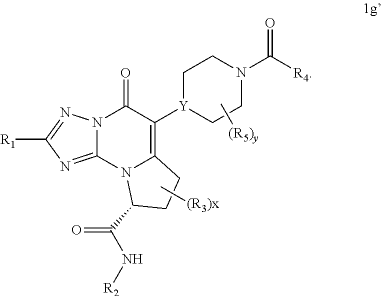

or formula 1h:

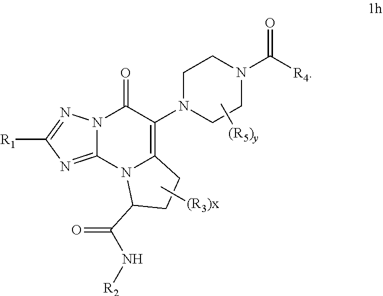

In particular, 1h is 1h′:

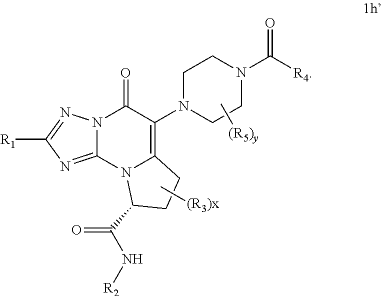

In one embodiment, there is provided a compound of formula (I), in particular formula (I′),

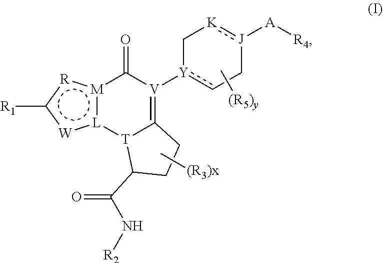

in particular

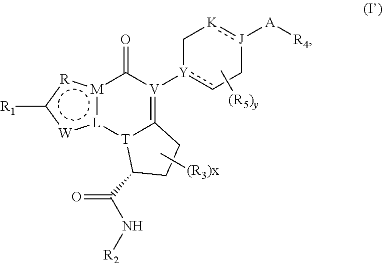

(more particularly

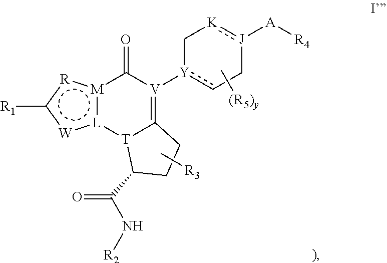

or a pharmaceutically acceptable salt thereof,
wherein
R, M, W, L, V and T are independently selected from C, CH and N,
to form subformulae 1a, 1b, 1c, 1d, 1e and 1f, in particular 1a′, 1b′, 1c′, 1d′, 1e′ and 1i′ as shown herein, in particular 1a′, more particularly 1h′ or 1g′ as described herein,
A is a linker which is —C(O)—;
Y is N, C or CH;
Y [symbol] means Y is linked via a single bond to the adjacent carbon atom when Y is CH, or Y is linked via a double bond to the adjacent atom when Y is C, and when Y [symbol] is a single bond, Y is carbon unsubstituted or substituted by OH or F; [symbol]
when Y is N, Y [symbol] is a single bond;
K [symbol] means K is linked via a single or double bond to the adjacent atom;
wherein:
   when K [symbol] is a double bond, Y [symbol] is a single bond, K is CH, J is C, and A is a linker which is —C(O)—;
or
   when K [symbol] is a single bond, K is selected from —CH₂—, —CH₂CH₂—, —NH— and a bond (to form a 5-membered ring:

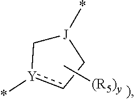

J is N, and A is a linker which is —C(O)—, (in particular K is —CH₂—, and J is N);
y is 0, 1, 2, 3 or 4;
R₅ is independently selected from:
   —(C₁-C₄)alkyl,
   —(C₃-C₅)cycloalkyl,
   and wherein two R₅ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S,
   when K [symbol] J is a carbon-nitrogen single bond, a R₅ substituent on K and on the adjacent carbon atom may join to form ring C:

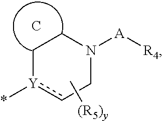

wherein ring C is a fused (C₃-C₆)cycloalkyl ring (preferably cyclobutyl), a fused (C₃-C₆)heterocyclyl ring or a fused phenyl ring, wherein said fused (C₃-C₆)heterocyclyl ring contains ring carbon atoms and one ring heteroatom selected from O, N and S,
   and wherein when ring C is a fused (C₃-C₆)cycloalkyl ring, said fused (C₃-C₆)cycloalkyl ring is unsubstituted or substituted with 1 or 2 R₄₀ groups, wherein said R₄₀ is selected from:
      (C₁-C₂)alkyl, wherein each (C₁-C₂)alkyl is independently unsubstituted or substituted by OH or 1, 2 or 3 halo,
      halo, in particular F,
      or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
      or wherein two R₄₀ substituents on adjacent carbon atoms join together with the carbon atoms to which they are attached, to form a fused cyclopropyl ring;
and wherein when K is —CH₂— and J is N, two R₅ substituents may join to form a (C₁-C₃)alkylene bridge or a heteroalkylene bridge, wherein said heteroalkylene bridge is one heteroatom selected from N and O, or is —CH₂—O—CH₂—;
R₁ is:
cycloalkenyl, wherein said cycloalkenyl is a partially unsaturated monocyclic ring containing 5 or 6 ring carbon atoms, and said cycloalkenyl is unsubstituted or substituted by 1, 2, 3 or 4, preferably 1 or 2, R₃₃, wherein R₃₃ is halo, and wherein said cycloalkenyl or halo-substituted cycloalkenyl is substituted by 0, 1 or 2 R₁₅ substituents,
or R₁ is heterocyclyl, wherein said heterocyclyl is a 5 or 6 membered fully saturated or partially unsaturated group comprising ring carbon atoms and 1 or 2 ring heteroatoms independently selected from N, NH, O and S, and wherein said heterocyclyl is unbridged or bridged, and said bridge is 1 or 2 carbon atoms, wherein said heterocyclyl is unsubstituted or substituted by 1, 2, 3 or 4, preferably 1 or 2, R₃₃, wherein R₃₃ is halo, and wherein said heterocyclyl or halo-substituted heterocyclyl is substituted by 0, 1 or 2 substituents independently selected from R₁₅, R₁₆, R₁₇, R₁₈, R₁₉, R₂₀, R₂₂ and R₂₃,
or said heterocyclyl or halo-substituted heterocyclyl is fused to a cyclopropyl ring, wherein said cyclopropyl ring is unsubstituted or substituted by 1, 2 or 3 F,
or said heterocyclyl or halo-substituted heterocyclyl has 2 substituents at the same ring carbon atom which join to form a cyclopropyl spiro ring,
or said heterocyclyl or halo-substituted heterocyclyl is fused with a (C₃-C₅)heterocycloalkyl ring, wherein said (C₃-C₅)heterocycloalkyl ring contains ring carbon atoms and 1 ring O atom;
or R₁ is heteroaryl, wherein said heteroaryl is a 5 or 6 membered fully unsaturated monocyclic group comprising ring carbon atoms and 1, 2, 3 or 4 ring heteroatoms independently selected from N, O and S, preferably 1 or 2 ring heteroatoms, preferably wherein the total number of ring S atoms does not exceed 1, and the total number of ring O atoms does not exceed 1,
and wherein said heteroaryl is unsubstituted or substituted by 1, 2 or 3 substituents independently selected from R₂₁ and R₃₀, wherein R₂₁ and R₃₀ are independently selected from halo and (C₁-C₄)alkyl, wherein said (C₁-C₄)alkyl is unsubstituted or substituted by 1, 2 or 3 halo,
or R₁ is phenyl, wherein said phenyl is unsubstituted or substituted by 1, 2, 3 or 4, preferably 1 or 2, R₃₃, wherein R₃₃ is halo, and wherein said phenyl or halo-substituted phenyl is substituted by 0, 1 or 2 R₁₅ substituents;
each R₁₅, R₁₆, R₁₇, R₁₈, R₁₉, R₂₀, R₂₂ and R₂₃ is independently selected from:
   halo
   (C₁-C₄)alkyl-O— unsubstituted or substituted by 1, 2 or 3 halo;
   (C₁-C₄)alkyl unsubstituted or substituted by OH, —O—(C₁-C₂)alkyl or 1, 2 or 3 halo,
   HOC(O)—(CH₂)ₙ—,
   H₃C—C(O)(CH₂)ₙ—,
   (C₁-C₄)alkyl-O—C(O)(CH₂)ₙ,
   ═O
   azetidinyl or pyrrolidinyl, wherein said azetidinyl and pyrrolidinyl are linked to the rest of the molecule via the N atom, and are each unsubstituted or substituted by 1 or 2 F,
   R₂₅(R₂₄)N—, wherein R₂₄ is H or (C₁-C₄)alkyl unsubstituted or substituted by 1, 2 or 3 halo, R₂₅ is H or (C₁-C₄)alkyl unsubstituted or substituted by 1, 2 or 3 halo,
   OH
wherein n is 0, 1 or 2,
with the proviso that when R₁ is a ring, then:
   each R₁ ring atom adjacent to the R₁ ring atom to which said R₁ ring is joined to the remainder of the molecule, is independently unsubstituted or substituted by halo only, in particular, independently unsubstituted or substituted with one F substituent, and
   said R₁ ring is linked to the remainder of the molecule via a R₁ ring nitrogen atom, or a R₁ ring carbon atom which is double-bonded to an adjacent R₁ ring atom;
R₂ is the moiety:

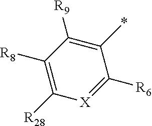

R₆ is selected from:
   H,
   halo,
   (C₁-C₄)alkyl unsubstituted or substituted by 1, 2 or 3 halo,
   (C₃-C₅)cycloalkyl unsubstituted or substituted by 1, 2 or 3 halo,
   —O—(C₁-C₄)alkyl unsubstituted or substituted by 1, 2 or 3 halo,
   OH, and
CN;
R₈ is selected from H, halo, and (C₁-C₄)alkyl unsubstituted or substituted by 1, 2 or 3 halo,
R₉ is selected from H, O—CH₃, OH, CN, CH₃ and halo;
R₂₈ is selected from:
   SF₅,
   H,
   —C(O)H,
   halo,
   (C₁-C₄)alkyl unsubstituted or substituted by 1, 2 or 3 halo,
   (C₁-C₄)alkynyl,
   (C₁-C₄)alkenyl,
   (C₃-C₅)cycloalkyl unsubstituted or substituted by 1, 2 or 3 halo, and
   OCF₃;
X is selected from C—R₇ and N, wherein R₇ is H or halo, or R₇ can join, together with R₂₈ or R₆, and the atoms to which they are attached, to form a fused (C₄-C₆)cycloalkyl ring, wherein said fused (C₄-C₆)cycloalkyl ring is unsubstituted or substituted by 1, 2 or 3 halo,
or
R₂ is selected from:

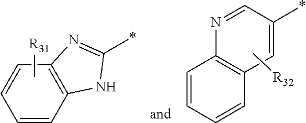

wherein
R₃₁ is selected from H, halo and CH₃,
R₃₂ is selected from H, halo and CH₃,
R₃ is selected from:
   halo, and
   (C₁-C₄)alkyl unsubstituted or substituted by 1, 2 or 3 substituents independently selected from halo and OH,
   or two R₃ substituents on the same ring carbon atom may join, together with the carbon atom to which they are attached, to form a cyclopropyl ring;
x is 0, 1 or 2;
R₄ is selected from:
—(C₁-C₄)alkyl;
-heteroaryl1, wherein said heteroaryl1 is a 5 or 6 membered, fully unsaturated, monocyclic ring comprising ring carbon atoms and 1, 2, 3 or 4 ring heteroatoms independently selected from N, O and S;
-heteroaryl2, wherein said heteroaryl2 is a 9 or 10 membered fused bicyclic ring comprising ring carbon atoms and 1, 2, 3 or 4 ring heteroatoms independently selected from N, O and S, and wherein both rings are fully unsaturated, or one ring is fully unsaturated, and the other is saturated or partially unsaturated, and wherein the heteroatoms may be in one or both rings;
-phenyl;
wherein heteroaryl1, heteroaryl2 and phenyl are each substituted by 1, 2 or 3 substituents independently selected from R₁₀, R₁₁, R₁₂, R₁₃ and R₁₄, wherein each R₁₀, R₁₁, R₁₂, R₁₃ and R₁₄ is independently selected from:
   H,
   halo,
   (C₁-C₄)alkyl unsubstituted or substituted by 1, 2 or 3 halo substituents,
   (C₁-C₂)alkyl substituted by —O—(C₁-C₂)alkyl or OH,
   —S—(C₁-C₃)alkyl,
   —O—(C₁-C₄)alkyl unsubstituted or substituted by 1, 2 or 3 halo substituents,
   OH,
   (C₃-C₅)cycloalkyl, wherein said (C₃-C₅)cycloalkyl is unsubstituted or substituted by 1 or 2 halo,
   —O—(C₃-C₅)cycloalkyl,
   —NR₃₄R₃₅ wherein R₃₄ and R₃₅ are independently selected from:
      H,
      (C₁-C₄)alkyl, wherein said (C₁-C₄)alkyl is unsubstituted or substituted by OH or —O(C₁-C₂)alkyl,
      and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
   CN,
   —(C₂-C₄)alkenyl,
   —(C₂-C₄)alkynyl,
   ═O
   —C(O)H, and
   —C(O)(C₁-C₄)alkyl;
with the proviso that:
   one OH substituent is present on heteroaryl1, heteroaryl2 and phenyl, and said OH is in the ortho position of the R₄ ring, relative to the position linking R₄ to linker -A-,
or
   one ═O substituent is present on heteroaryl1 and heteroaryl2;
and the remaining R₁₀, R₁₁, R₁₂, R₁₃ and R₁₄ are defined as herein,
and * indicates a point of attachment. Particular embodiments of said compound of formula (I), in particular formula (I′) or (I′″), are described herein.

In one embodiment, there is provided a compound of formula (1g) or a pharmaceutically acceptable salt thereof,

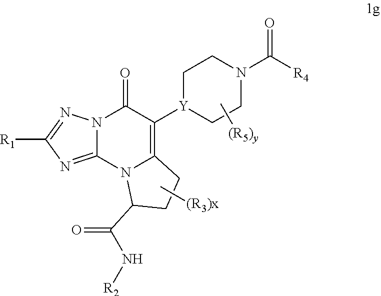

wherein R₁ is selected from:

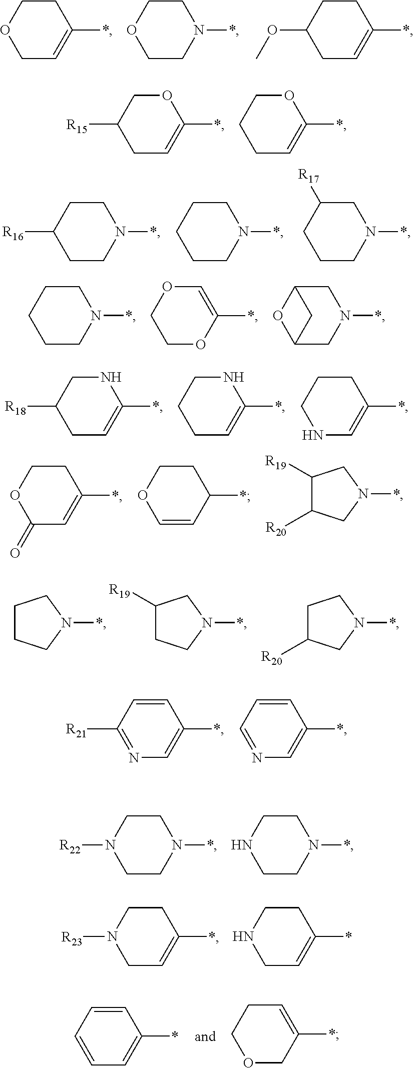

R₁₅ is F;
R₁₆ is R₂₅(R₂₄)N—;
R₁₇ is F;
R₁₈ is F;
R₁₉ is F;
R₂₀ is F;
R₂₁ is CH₃;
R₂₂ is CF₃, CHF₂CH₂, HOC(O)—CH₂—, H₃C—C(O)—, (H₃C)₃C—O—C(O)—;
R₂₃ is CF₃, CHF₂CH₂—, (H₃C)₃C—O—C(O)—;
R₂₄ is CH₃; and
R₂₅ is CHF₂CH₂—;
R₂ is the moiety:

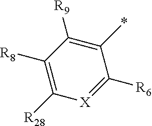

wherein
R₆ is selected from H, halo, (C₁-C₄)alkyl unsubstituted or substituted by 1, 2 or 3 halo;
R₈ is selected from H, halo, (C₁-C₄)alkyl unsubstituted or substituted by 1, 2 or 3 halo;
R₉ is selected from H, O—CH₃, OH, CN, CH₃ and halo;
R₂₈ is selected from SF₅, halo, (C₁-C₄)alkyl unsubstituted or substituted by 1, 2 or 3 halo and —C(O)H;
X is selected from C—R₇ and N; and
R₇ is selected from H and halo;
R₃ is (C₁-C₄)alkyl unsubstituted or substituted by 1, 2 or 3 substituents independently selected from halo and OH;
x is 0 or 1;
Y is CH or N, in particular N;
y is 0, 1 or 2;
R₅ is selected from CH₃;
or wherein in the moiety:

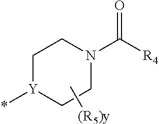

two R₅ substituents on adjacent carbon atoms join to form ring C:

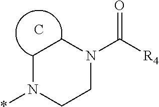

wherein ring C is a fused (C₃-C₆)cycloalkyl ring, in particular a fused cyclobutyl ring, and said fused (C₃-C₆)cycloalkyl ring is unsubstituted or substituted with 1 or 2 R₄₀ groups, wherein said R₄₀ is selected from:
   (C₁-C₂)alkyl, wherein each (C₁-C₂)alkyl is independently unsubstituted or substituted by OH or 1, 2 or 3 halo,
   halo, in particular F,
   or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
   or wherein two R₄₀ substituents on adjacent carbon atoms join together with the carbon atoms to which they are attached, to form a fused cyclopropyl ring;
and ring C in particular, is

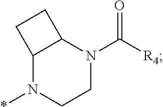

R₄ is selected from:
CH₃,

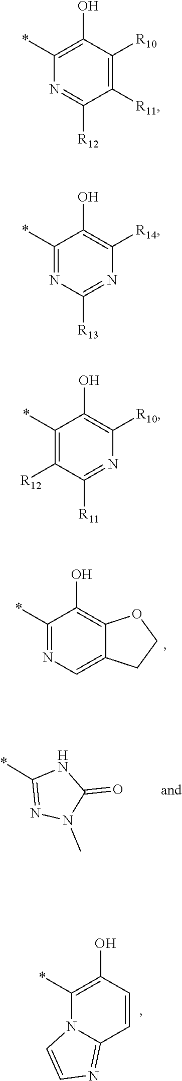

in particular

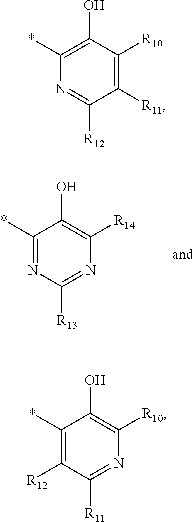

especially

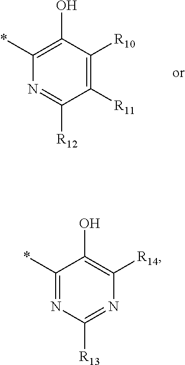

wherein
R₁₀ is selected from H, halo, (C₁-C₂)alkyl unsubstituted or substituted by 1, 2 or 3 halo substituents, —O—(C₁-C₂)alkyl unsubstituted or substituted by 1, 2 or 3 halo substituents;
R₁₁ is selected from H, halo, (C₁-C₂)alkyl unsubstituted or substituted by 1, 2 or 3 halo substituents;
R₁₂ is selected from H, halo, (C₁-C₂)alkyl unsubstituted or substituted by 1, 2 or 3 halo substituents;
R₁₃ is selected from H, —S—CH₃, halo, (C₁-C₂)alkyl unsubstituted or substituted by 1, 2 or 3 halo substituents; and
R₁₄ is selected from H, halo, (C₁-C₂)alkyl unsubstituted or substituted by 1, 2 or 3 halo substituents, O—(C₁-C₂)alkyl unsubstituted or substituted by 1, 2 or 3 halo substituents, and cyclopropyl.

In particular, R₁, R₂, R₃, x, R₅, y, Y and R₄ are as described in the embodiments herein.

In another embodiment, there is provided a compound of formula (1 h) or a pharmaceutically acceptable salt thereof,

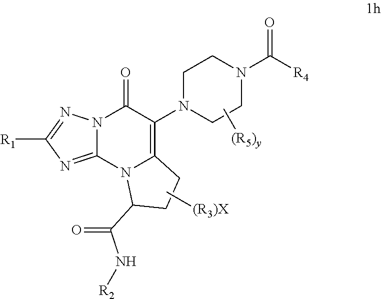

wherein:
R₁ is selected from:

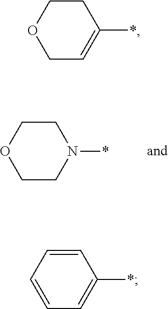

R₂ is selected from:

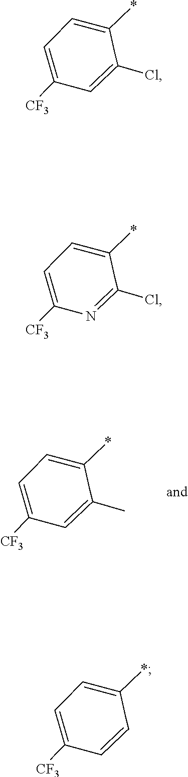

R₃ is CH₃;
x is 0 or 1;
R₄ is selected from:
—CH₃,

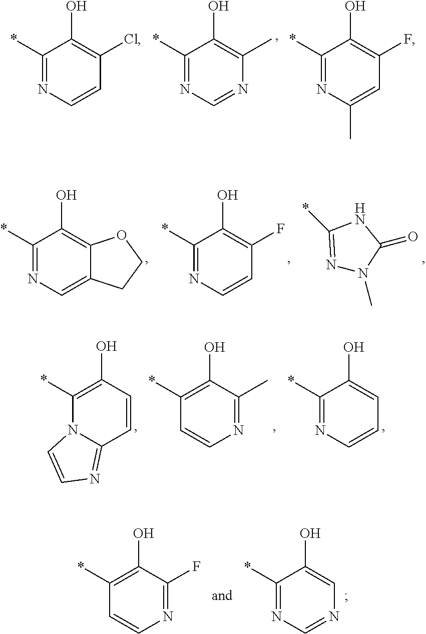

y is 0 or 1;
R₅ is selected from CH₃;
or wherein the moiety:

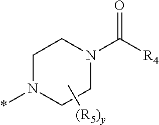

is

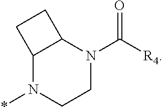

In particular, R₁, R₂, R₃, x, R₅, y and R₄ are as described in the embodiments herein.

There is further provided an intermediate compound or a salt thereof, as used in the chemical synthesis of a compound of formula (I) as described herein.

In another aspect, there is provided a process, or a process step, in the synthesis of a compound of formula (I) as described herein.

For example, in one embodiment of the invention there is provided an intermediate compound
of Formula FN2:

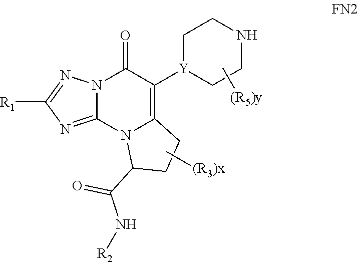

wherein Y, R₁, R₂, R₃, x, R₅, and y are as defined herein.

For example, an intermediate compound N2:

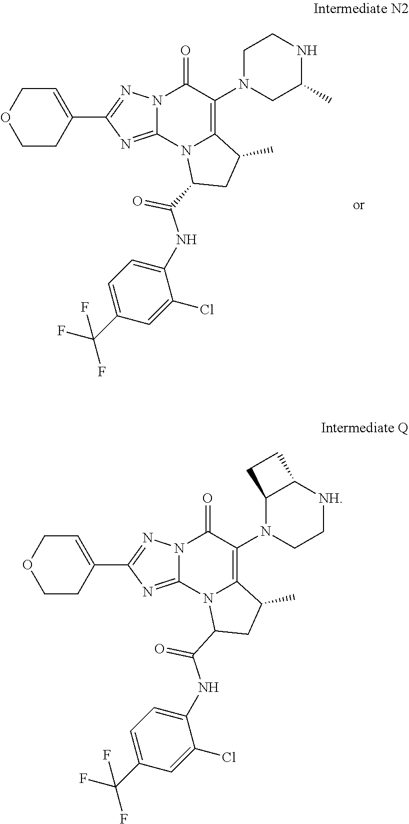 [SMILES: C[C@@H]1CN(c2c3n(c4nc(C5=CCOCC5)nn4c2=O)[C@@H](C(=O)Nc2ccc(C(F)(F)F)cc2Cl)C[C@H]3C)CCN1 ; C[C@@H]1CC(C(=O)Nc2ccc(C(F)(F)F)cc2Cl)n2c1c(N1CCN[C@H]3CC[C@@H]31)c(=O)n1nc(C3=CCOCC3)nc21]

In another embodiment of the invention there is provided an intermediate compound of formula FM:

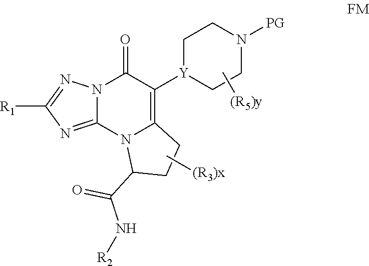

wherein Y, R₁, R₂, R₃, x, R₅, and y are as defined herein, and PG is any suitable protecting group, including BOC (tert-butyloxycarbonyl). Such suitable protecting groups are known to the skilled person.

For example, there is provided an intermediate compound M1, M2, M3, M4 or QPG:

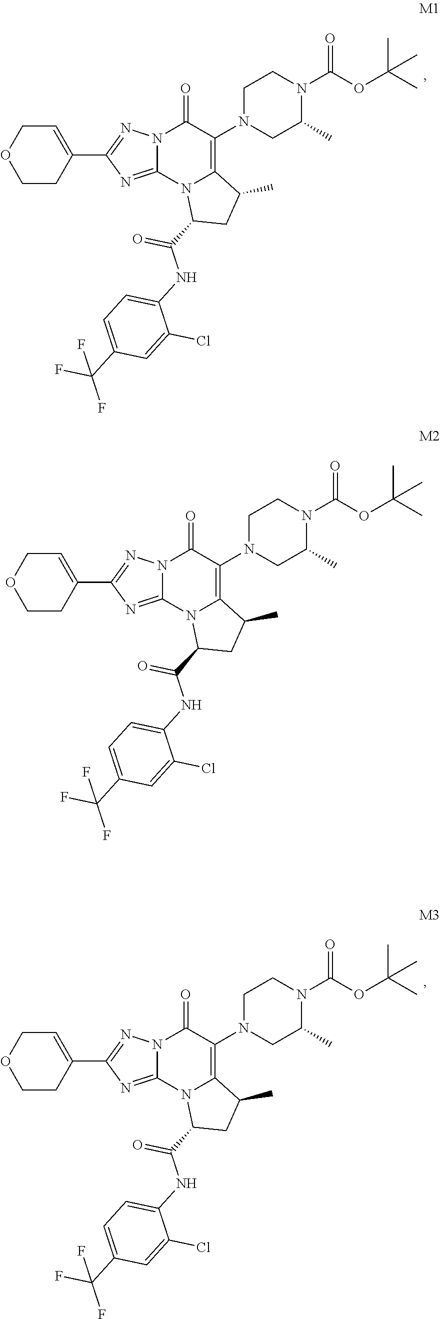 [SMILES: C[C@@H]1C[C@H](C(=O)Nc2ccc(C(F)(F)F)cc2Cl)n2c1c(N1CCN(C(=O)OC(C)(C)C)[C@H](C)C1)c(=O)n1nc(C3=CCOCC3)nc21 ; C[C@@H]1CN(c2c3n(c4nc(C5=CCOCC5)nn4c2=O)[C@H](C(=O)Nc2ccc(C(F)(F)F)cc2Cl)C[C@@H]3C)CCN1C(=O)OC(C)(C)C ; C[C@@H]1CN(c2c3n(c4nc(C5=CCOCC5)nn4c2=O)[C@@H](C(=O)Nc2ccc(C(F)(F)F)cc2Cl)C[C@@H]3C)CCN1C(=O)OC(C)(C)C]

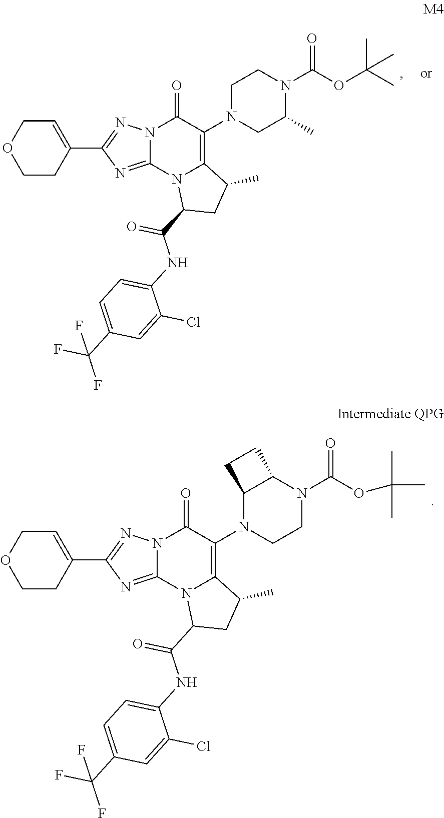 [SMILES: C[C@@H]1C[C@@H](C(=O)Nc2ccc(C(F)(F)F)cc2Cl)n2c1c(N1CCN(C(=O)OC(C)(C)C)[C@H](C)C1)c(=O)n1nc(C3=CCOCC3)nc21 ; C[C@@H]1CC(C(=O)Nc2ccc(C(F)(F)F)cc2Cl)n2c1c(N1CCN(C(=O)OC(C)(C)C)[C@H]3CC[C@@H]31)c(=O)n1nc(C3=CCOCC3)nc21]

In another embodiment of the invention there is provided an intermediate compound of formula FL:

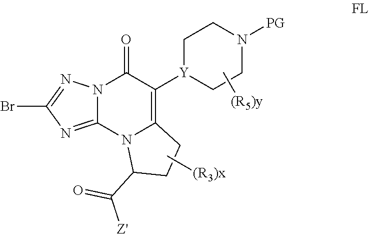

wherein Y, R₃, x, R₅, and y are as defined herein, wherein Z′ is OH or O—C(CH₃)₃. PG is any suitable protecting group, including BOC (tert-butyloxycarbonyl). Such suitable protecting groups are known to the skilled person.

For example, there is provided an intermediate compound L:

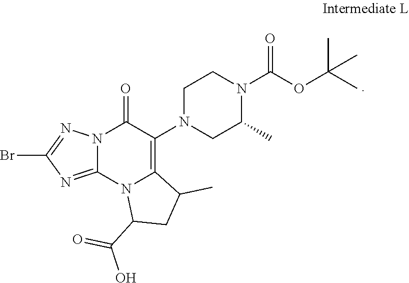 [SMILES: CC1CC(C(=O)O)n2c1c(N1CCN(C(=O)OC(C)(C)C)[C@H](C)C1)c(=O)n1nc(Br)nc21]

In another embodiment of the invention there is provided an intermediate compound of formula:

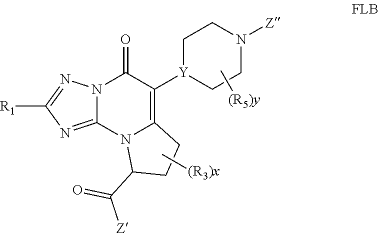

wherein Y, R₁, R₃, x, R₅ and y are as defined herein, Z′ is OH or O—C(CH₃)₃ and Z″ is H or PG, wherein PG is any suitable protecting group, including BOC (tert-butyloxycarbonyl). Suitable protecting groups are known to the skilled person.

For example, there is provided an intermediate compound LB:

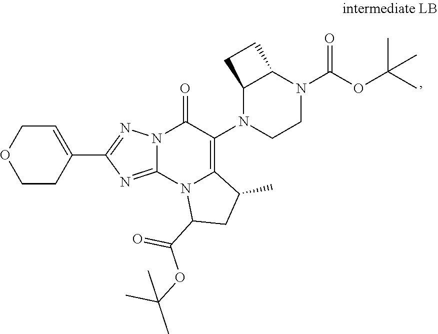 [SMILES: C[C@@H]1CC(C(=O)OC(C)(C)C)n2c1c(N1CCN(C(=O)OC(C)(C)C)[C@H]3CC[C@@H]31)c(=O)n1nc(C3=CCOCC3)nc21]

or intermediate Q4:

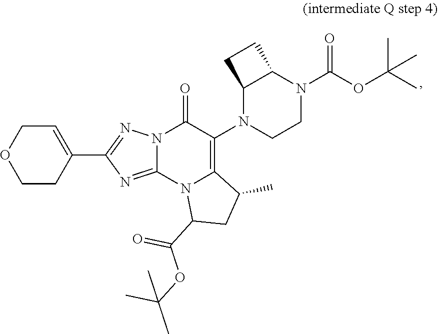 [SMILES: C[C@@H]1CC(C(=O)OC(C)(C)C)n2c1c(N1CCN(C(=O)OC(C)(C)C)[C@H]3CC[C@@H]31)c(=O)n1nc(C3=CCOCC3)nc21]

or intermediate Q5:

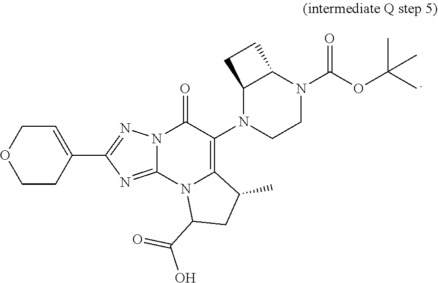 [SMILES: C[C@@H]1CC(C(=O)O)n2c1c(N1CCN(C(=O)OC(C)(C)C)[C@H]3CC[C@@H]31)c(=O)n1nc(C3=CCOCC3)nc21]

In another embodiment there is provided an intermediate compound U:

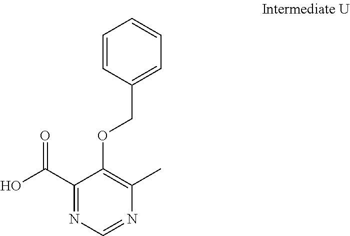 [SMILES: Cc1ncnc(C(=O)O)c1OCc1ccccc1]

In another embodiment of the invention there is provided an intermediate compound of formula FQ:

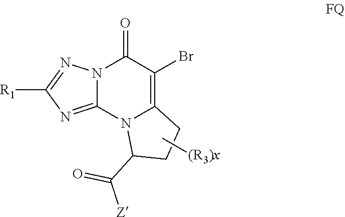

wherein R₁, R₃ and x are as defined herein, and Z′ is OH or O—C(CH₃)₃.

For example, there is provided an intermediate compound Q3.1, Q3.2, Q3.3 or Q3.4;

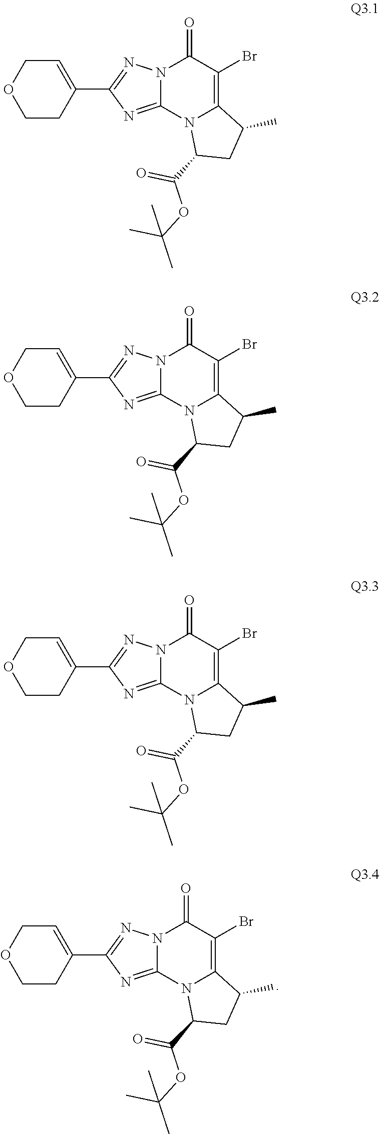

In a further aspect, the invention is as claimed or described herein.

### Formulations

In another aspect, the present invention provides a pharmaceutical composition comprising a compound of the present invention, or a pharmaceutically acceptable salt thereof, and a pharmaceutically acceptable carrier. In a further embodiment, the composition comprises at least two pharmaceutically acceptable carriers, such as those described herein. The pharmaceutical composition can be formulated for particular routes of administration such as oral administration, parenteral administration (e.g. by injection, infusion, transdermal or topical administration), and rectal administration, in particular oral administration. Topical administration may also pertain to inhalation or intranasal application. The pharmaceutical compositions of the present invention can be made up in a solid form (including, without limitation, capsules, tablets, pills, granules, powders or suppositories), or in a liquid form (including, without limitation, solutions, suspensions or emulsions). Tablets may be either film coated or enteric coated according to methods known in the art. Typically, the pharmaceutical compositions are tablets or gelatin capsules comprising the active ingredient together with one or more of:
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and
e) absorbents, colorants, flavors and sweeteners.

Compounds intended for parenteral or oral administration can be solubilized using various methods including nano-suspensions, solid dispersions and liposomes (van Hoogevest P., Xiangli L., and Alfred F. “Drug delivery strategies for poorly water-soluble drugs: the industrial perspective” Expert Opinion on Drug Delivery 2011, 8(11), 1481-1500).

Solid dispersion technologies have been used to improve the dissolution characteristics and bioavailability of orally administered drugs (Dhirendra K et al: ‘Solid dispersions: A review”, Pakistan Journal of Pharmaceutical Sciences, Faculty of Pharmacy, University of Karachi, Pakistan, vol. 22, no. 2. 30 April 200, pages 234-246).

Typical approaches to solubilize compounds for parenteral administration are the optimization of the pH or the use of co-solvents (e.g. PEG300, PEG400, propylene glycol, or ethanol). If these approaches are, for any reason, not feasible, the use of surfactants may be considered (e.g. Tween® 80 or Cremophor EL®). Cyclodextrins are established as safe solubilizing agents. Compounds with a high solubility in natural oils (e.g. propofol) may be solubilized in parenteral fat emulsions.

There is also provided a pharmaceutical composition comprising a compound of formula (I) as described herein, or a pharmaceutically acceptable salt thereof, and one or more pharmaceutically acceptable carriers.

### Uses

The compounds of formula (I) of the present invention in free form or in pharmaceutically acceptable salt form, exhibit valuable pharmacological properties, e.g. WRN inhibiting properties, e.g. as indicated in in vitro and in vivo tests as provided in the next sections, and are therefore indicated for therapy, or for use as research chemicals, e.g. as a chemical probe, and as tool compounds.

In another aspect of the invention there is provided a compound of formula (I), or a salt thereof, as described herein, for use as a research chemical, for example a tool compound or chemical probe, in particular for research on WRN. In another embodiment there is provided the use of a compound of formula (I), or a salt thereof, as described herein, as a research chemical, for example tool compound or chemical probe, in particular for research on WRN.

There is also provided a compound of formula (I) as described herein, or a pharmaceutically acceptable salt thereof, for use in the treatment of cancer. Cancers that may be treated by WRN inhibition include cancers that are characterized as microsatellite instability-high (MSI-H) or mismatch repair deficient (dMMR). In particular, a compound of formula (I) as described herein, or a pharmaceutically acceptable salt thereof, may be useful in the treatment of a cancer that is characterized as microsatellite instability-high (MSI-H) or mismatch repair deficient (dMMR).

There is also provided a compound of formula (I) as described herein, or a pharmaceutically acceptable salt thereof, for use as a medicament. In particular, said use is:
for the treatment of a disease that is treated by WRN inhibition,
for the treatment of cancer,
for the treatment of cancer that is characterized as microsatellite instability-high (MSI-H) or mismatch repair deficient (dMMR),
for the treatment of cancer that is characterized as microsatellite instability-high (MSI-H) or mismatch repair deficient (dMMR), such as colorectal, gastric, prostate, endometrial, adrenocortical, uterine, cervical, esophageal, breast, kidney and ovarian cancer,
for the treatment of cancer that is characterized as microsatellite instability-high (MSI-H) or mismatch repair deficient (dMMR) is selected from colorectal, gastric, prostate and endometrial cancer, or
for the treatment of cancer wherein the cancer characterized as microsatellite instability-high (MSI-H) or mismatch repair deficient (dMMR) is selected from uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma, prostate cancer and ovarian serous cystadenocarcinoma.

There is also provided a method of:
modulating WRN activity in a subject, wherein the method comprises administering to the subject a therapeutically effective amount of the compound of formula (I) as described herein, or a pharmaceutically acceptable salt thereof,
inhibiting WRN in a subject, wherein the method comprises administering to the subject a therapeutically effective amount of the compound of formula (I) as described herein, or a pharmaceutically acceptable salt thereof,
treating a disorder or disease which can be treated by WRN inhibition in a subject, comprising administering to the subject a therapeutically effective amount of the compound of formula (I) as described herein, or a pharmaceutically acceptable salt thereof,
treating cancer in a subject, comprising administering to the subject a therapeutically effective amount of the compound of formula (I) as described herein, or a pharmaceutically acceptable salt thereof,
treating cancer in a subject, comprising administering a compound of formula (I) as described herein, or a pharmaceutically acceptable salt thereof, wherein the cancer is characterized as microsatellite instability-high (MSI-H) or mismatch repair deficient (dMMR). In particular, the cancer characterized as microsatellite instability-high (MSI-H) or mismatch repair deficient (dMMR) is selected from colorectal, gastric, prostate, endometrial, adrenocortical, uterine, cervical, esophageal, breast, kidney and ovarian cancer. More particularly, the cancer characterized as microsatellite instability-high (MSI-H) or mismatch repair deficient (dMMR) is selected from colorectal, gastric, prostate and endometrial cancer. Examples include uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma, prostate cancer and ovarian serous cystadenocarcinoma.

There is also provided the use of a compound of formula (I) as described herein, or a pharmaceutically acceptable salt thereof:
in therapy,
in the manufacture of a medicament,
in the manufacture of a medicament for the treatment of cancer. In particular, said cancer is characterized as microsatellite instability-high (MSI-H) or mismatch repair deficient (dMMR),
in the manufacture of a medicament for treatment of a disease which may be treated by WRN inhibition,
wherein in particular, the cancer is characterized by microsatellite instability-high (MSI-H) or mismatch repair deficient (dMMR), for example colorectal, gastric, prostate, endometrial, adrenocortical, uterine, cervical, esophageal, breast, kidney and ovarian cancer, in particular, colorectal, gastric, prostate or endometrial cancer, or uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma and ovarian serous cystadenocarcinoma.

In some embodiments, the subject has or is identified as having a microsatellite instable (MSI-H) cancer, e.g., in reference to a control, e.g., a normal, subject. In one embodiment, the subject has MSI-H advanced solid tumors, a colorectal cancer (CRC), endometrial, uterine, stomach or other MSI-H cancer. In some embodiments, the subject has a colorectal (CRC), endometrial or stomach cancer, which cancer has or is identified as having a microsatellite instability (MSI-H), e.g., in reference to a control, e.g., a normal, subject. Such identification techniques are known in the art.

### Forms

Depending on the choice of the starting materials and procedures, the compounds can be present in the form of one of the possible stereoisomers or as mixtures thereof, for example as pure optical isomers, or as stereoisomer mixtures, such as racemates and diastereoisomer mixtures, depending on the number of asymmetric carbon atoms. The present invention is meant to include all such possible stereoisomers, including racemic mixtures, diasteriomeric mixtures and optically pure forms. Optically active (R)- and (S)-stereoisomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

As used herein, the terms “salt” or “salts” refers to an acid addition or base addition salt of a compound of the present invention. “Salts” include in particular “pharmaceutical acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like.

Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

In a particular embodiment, there is provided a sodium salt of a compound described herein.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

In another aspect, the present invention provides compounds of the present invention in acetate, ascorbate, adipate, aspartate, benzoate, besylate, bromide/hydrobromide, bicarbonate/carbonate, bisulfate/sulfate, camphorsulfonate, caprate, chloride/hydrochloride, chlortheophyllonate, citrate, ethandisulfonate, fumarate, gluceptate, gluconate, glucuronate, glutamate, glutarate, glycolate, hippurate, hydroiodide/iodide, isethionate, lactate, lactobionate, laurylsulfate, malate, maleate, malonate, mandelate, mesylate, methylsulphate, mucate, naphthoate, napsylate, nicotinate, nitrate, octadecanoate, oleate, oxalate, palmitate, pamoate, phosphate/hydrogen phosphate/dihydrogen phosphate, polygalacturonate, propionate, sebacate, stearate, succinate, sulfosalicylate, sulfate, tartrate, tosylate trifenatate, trifluoroacetate or xinafoate salt form.

Any formula given herein is intended to represent unlabeled forms as well as isotopically labeled forms of the compounds. Isotopically labeled compounds have structures depicted by the formulae given herein except that one or more atoms are replaced by an atom having a selected atomic mass or mass number. Isotopes that can be incorporated into compounds of the invention include, for example, isotopes of hydrogen. For example, the invention includes deuterated forms of the exemplified compounds disclosed herein.

For example, one or more H atoms on the ring:

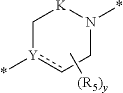

may be replaced by deuterium,
or one or more atoms on the R₁ moiety may be replaced by deuterium:

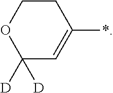

Further, incorporation of certain isotopes, particularly deuterium (i.e., ²H or D) may afford certain therapeutic advantages resulting from greater metabolic stability, for example increased in vivo half-life or reduced dosage requirements or an improvement in therapeutic index or tolerability. It is understood that deuterium in this context is regarded as a substituent of a compound of the present invention. The concentration of deuterium, may be defined by the isotopic enrichment factor. The term “isotopic enrichment factor” as used herein means the ratio between the isotopic abundance and the natural abundance of a specified isotope. If a substituent in a compound of this invention is denoted as being deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It should be understood that the term “isotopic enrichment factor” can be applied to any isotope in the same manner as described for deuterium.

Other examples of isotopes that can be incorporated into compounds of the invention include isotopes of hydrogen, carbon, nitrogen, oxygen, phosphorous, fluorine, and chlorine, such as ³H, ¹¹C, ¹³C, ¹⁴C, ¹⁵N, ¹⁸F, ³¹P, ³²P, ³⁵S, ³⁶Cl, ¹²³I, ¹²⁴I, ¹²⁵I respectively. Accordingly it should be understood that the invention includes compounds that incorporate one or more of any of the aforementioned isotopes, including for example, radioactive isotopes, such as ³H and ¹⁴C, or those into which non-radioactive isotopes, such as ²H and ¹³C are present. Such isotopically labelled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of the present invention can generally be prepared by conventional techniques known to those skilled in the art or by processes analogous to those described in the accompanying Examples and Preparations using an appropriate isotopically-labeled reagents in place of the non-labeled reagent previously employed.

### Definitions

A ‘compound of the present invention’ or a ‘compound of formula (I)’ or a ‘compound of formula 1a’ etc., includes a zwitterion thereof, a non-zwitterion thereof (non-charged form), or a pharmaceutically acceptable salt of said zwitterionic or non-zwitterionic form thereof. ‘zwitterion’ or ‘zwitterionic form’ means a compound containing both positive and negatively charged functional groups.

For example, the compound of formula (I) described herein can include the following forms, wherein R₄ is the zwitterionic form (c) or non-zwitterionic form (d),

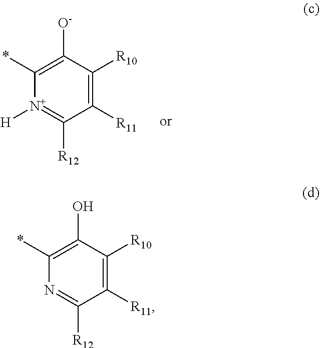

or a mixture thereof.

The compound of formula (I) described herein can also include the following forms, wherein R₄ is the zwitterionic form (a) or (b) or the non-zwitterionic form (e),

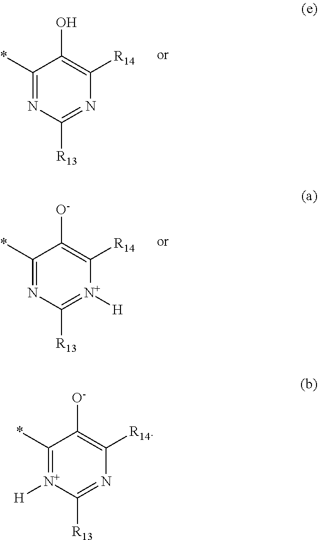

or a mixture of two thereof, or a mixture of all three thereof.
halo means fluoro, chloro or bromo, particularly fluoro or chloro.

Alkyl, and alkoxy groups, containing the requisite number of carbon atoms, can be unbranched or branched. Examples of alkyl include, but are not limited to, methyl, ethyl, n-propyl, i-propyl, n-butyl, i-butyl, sec-butyl and t-butyl. Examples of alkoxy include methoxy, ethoxy, n-propoxy, i-propoxy, n-butoxy, i-butoxy, sec-butoxy and t-butoxy.

‘═O’ means an oxo substituent.

When R₁ is substituted or unsubstituted cycloalkenyl, said cycloalkenyl includes, but is not limited to, groups such as cyclohexenyl, in particular cyclohex-1-en-1-yl.

When R₁ is substituted or unsubstituted heterocyclyl, said heterocyclyl includes, but is not limited to, groups such as morpholinyl, piperidinyl, pyrrolidinyl, 6-oxa-3-azabicyclo[3.1.1]heptan-3-yl, 5,6-dihydro-1,4-dioxin-2-yl, dihydropyranyl, in particular 3,4-dihydro-2H-pyran-6-yl, 5,6-dihydro-2H-pyran-3-yl and 3,6-dihydro-2H-pyran-4-yl, piperazinyl, tetrahydropyridinyl, such as 1,4,5,6-tetrahydropyridin-3-yl and 1,2,3,6-tetrahydropyridin-4-yl and dihydropyridinyl, such as 3,6-dihydropyridinyl.

When R₁ is heteroaryl, said heteroaryl is a 5 or 6 membered fully unsaturated (which includes aromatic), monocyclic group comprising ring carbon atoms and 1, 2, 3 or 4 ring heteroatoms independently selected from N, O and S, preferably 1 or 2 ring heteroatoms, preferably wherein the total number of ring S atoms does not exceed 1 and the total number of ring O atoms does not exceed 1. When R₁ is substituted or unsubstituted heteroaryl, said heteroaryl includes, but is not limited to, substituted or unsubstituted groups such as pyridinyl, in particular pyridin-3-yl.

-‘heteroaryl1’ is a 5 or 6 membered, fully unsaturated (which includes aromatic) monocyclic ring comprising ring carbon atoms and 1, 2, 3 or 4 ring heteroatoms independently selected from N, O and S. Preferably, the total number of ring S atoms does not exceed 1 and the total number of ring O atoms does not exceed 1. In particular, said heteraryl1 comprises ring carbon atoms and one or two nitrogen atoms only. Heteroaryl1 includes, but is not limited to, pyrrolyl, furanyl, thiophenyl, pyrazolyl, imidazolyl, isoxazolyl, oxadiazolyl, oxazolyl, isothiazolyl, thiazolyl, thiadiazolyl, tetrazolyl, pyridinyl, pyridazinyl, pyrimidinyl, triazolyl and pyrazinyl, in particular pyridyl, pyrimidinyl and triazolyl.

-‘heteroaryl2’, wherein said heteroaryl2 is a 9 or 10 membered fused bicyclic ring comprising ring carbon atoms and 1, 2, 3 or 4 ring heteroatoms independently selected from N, O and S, and wherein both rings are fully unsaturated (which includes aromatic), or one ring is fully unsaturated (which includes aromatic), and the other is saturated or partially unsaturated, and wherein the heteroatoms may be in one or both rings. Preferably, the total number of ring S atoms does not exceed 1 and the total number of ring O atoms does not exceed 1. In particular, the ring which is linked to the rest of the molecule via linker -A- is fully unsaturated. Heteroaryl2 includes, but is not limited to, benzofuranyl, benzothiophenyl, indolyl, benzimidazolyl, indazolyl, benzotriazolyl, pyrrolopyridinyl, imidazopyridinyl, pyrazololpyridinyl, isoindolyl, indazolyl, purinyl, indolininyl, imidazopyridinyl, pyrazolopyridinyl, pyrrolopyridazinyl, pyrrolopyridinyl, imidazopyrimidinyl, quinolinyl, isoquinolinyl, cinnolinyl, quinazolinyl, quinoxalinyl, phthalazinyl, naphthyridinyl, pyridopyrimidinyl, pyridopyrazinyl, pyrimidopyrimidinyl, pyrazinopyrazinyl, hydropyranopyridinyl, in particular hydrofuropyridinyl especially dihydrofuropyridinyl, and imidazopyridinyl.

The invention includes all tautomeric forms of the compounds of formula (I).

For example, when heteroaryl1 and heteroaryl2 are substituted by ═O they may form tautomers, for example as follows:

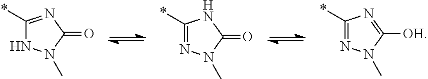

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to colorectal, gastric, endometrial, prostate, adrenocortical, uterine, cervical, esophageal, breast, kidney, ovarian cancer and the like.

The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors. ‘WRN inhibitor’ or ‘WRN helicase inhibitor’ as used herein means a compound that inhibits Werner Syndrome RecQ DNA helicase (WRN). The term “WRN” as used herein refers to the protein of Werner Syndrome RecQ DNA helicase. The term “WRN” includes mutants, fragments, variants, isoforms, and homologs of full-length wild-type WRN. In one embodiment, the protein is encoded by the WRN gene (Entrez gene ID 7486; Ensembl ID ENSG00000165392). Exemplary WRN sequences are available at the Uniprot database under accession number Q14191.

‘disease or condition mediated by WRN’ includes a disease or condition, such as cancer, which is treated by WRN inhibition. In particular this can include cancers characterized as microsatellite instability-high (MSI-H) or mismatch repair deficient (dMMR).

‘microsatellite unstable cancer’, microsatellite instability-high cancer’, ‘microsatellite high cancer’ and ‘MSI-high cancer’ ‘MSI^{hi}’ and ‘MSI-H’ when used herein, are used interchangeably, and describe cancers that have a high number of alterations in the length of simple repetitive genomic sequences within microsatellites.

The determination of MSI-H or dMMR tumor status for patients can be performed using, e.g., polymerase chain reaction (PCR) tests for MSI-H status or immunohistochemistry (IHC) tests for dMMR. Methods for identification of MSI-H or dMMR tumor status are described, e.g., in Ryan et al. Crit Rev Oncol Hematol. 2017; 116:38-57; Dietmaier and Hofstadter. Lab Invest 2001, 81:1453-1456; and Kawakami et al. Curr Treat Options Oncol. 2015; 16(7): 30).

Microsatellite instability can be found in colorectal cancer, gastric cancer and endometrial cancer in particular, but also in adrenocortical, uterine, cervical, esophageal, breast, kidney, prostate and ovarian cancers. Examples of microsatellite high cancers include uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma and ovarian serous cystadenocarcinoma.

A cancer that has “defective mismatch repair” (dMMR) or “dMMR character” includes cancer types associated with documented MLH1, PMS2, MSH2, MSH3, MSH6, MLH3, and PMS1 mutations or epigenetic silencing, microsatellite fragile sites, or other gene inactivation mechanisms, including but not limited to cancers of the lung, breast, kidney, large intestine, ovary, prostate, upper aerodigestive tract, stomach, endometrium, liver, pancreas, haematopoietic and lymphoid tissue, skin, thyroid, pleura, autonomic ganglia, central nervous system, soft tissue, pediatric rhabdoid sarcomas, melanomas and other cancers. A cell or cancer with “defective” mismatch repair has a significantly reduced (e.g., at least about 25%, 30%, 40%, 50%, 60%, 70%, 80% or 90% decrease) amount of mismatch repair. In some cases, a cell or cancer which is defective in mismatch repair will perform no mismatch repair.

As used herein, the term “pharmaceutical composition” refers to a compound of the invention, or a pharmaceutically acceptable salt thereof, together with at least one pharmaceutically acceptable carrier, in a form suitable for oral or parenteral administration.

As used herein, the term “pharmaceutically acceptable carrier” refers to a substance useful in the preparation or use of a pharmaceutical composition and includes, for example, suitable diluents, solvents, dispersion media, surfactants, antioxidants, preservatives, isotonic agents, buffering agents, emulsifiers, absorption delaying agents, salts, drug stabilizers, binders, excipients, disintegration agents, lubricants, wetting agents, sweetening agents, flavoring agents, dyes, and combinations thereof, as would be known to those skilled in the art (see, for example, Remington The Science and Practice of Pharmacy, 22nd Ed. Pharmaceutical Press, 2013, pp. 1049-1070).

The terms “synthetic lethality,” and “synthetic lethal” are used to refer to reduced cell viability and/or a reduced rate of cell proliferation caused by a combination of mutations or approaches to cause loss of function (e.g., RNA interference or protein function inhibition) in two or more genes but not by the loss of function of only one of these genes.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc.

In one embodiment, the term “a therapeutically effective amount” refers to the amount of the compound of the present invention that, when administered to a subject, is effective to (1) at least partially alleviate, prevent and/or ameliorate a condition, or a disorder or a disease (i) mediated by WRN, or (ii) associated with WRN activity, or (iii) characterized by activity (normal or abnormal) of WRN; or (2) reduce or inhibit the activity of WRN.

In another embodiment, the term “a therapeutically effective amount” refers to the amount of the compound of the present invention that, when administered to a cell, or a tissue, or a non-cellular biological material, or a medium, is effective to at least partially reducing or inhibiting the activity of WRN, or reducing WRN protein levels.

As used herein, the term “subject” refers to primates (e.g., humans, male or female), dogs, rabbits, guinea pigs, pigs, rats and mice. In certain embodiments, the subject is a primate, a rat or a mouse. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers to alleviating or ameliorating the disease or disorder (i.e., slowing or arresting the development of the disease or at least one of the clinical symptoms thereof); or alleviating or ameliorating at least one physical parameter or biomarker associated with the disease or disorder, including those which may not be discernible to the patient.

As used herein, the term “prevent”, “preventing” or “prevention” of any disease or disorder refers to the prophylactic treatment of the disease or disorder; or delaying the onset or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context. ‘May join’ means joins or does not join.

‘May be replaced by deuterium’ means is replaced by deuterium, or is not replaced by deuterium.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

### Isomeric Forms

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein a compound of the present invention can be in the form of one of the possible stereoisomers, rotamers, atropisomers, tautomers or mixtures thereof, for example, as substantially pure geometric (cis or trans) stereoisomers, diastereomers, optical isomers (antipodes), racemates or mixtures thereof.

Any resulting mixtures of stereoisomers can be separated on the basis of the physicochemical differences of the constituents, into the pure or substantially pure geometric or optical isomers, diastereomers, racemates, for example, by chromatography and/or fractional crystallization.

Any resulting racemates of compounds of the present invention or of intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′p-toluoyl tartaric acid, mandelic acid, malic acid or camphor-10-sulfonic acid. Racemic compounds of the present invention or racemic intermediates can also be resolved by chiral chromatography, e.g., high pressure liquid chromatography (HPLC) using a chiral adsorbent.

Compounds of the invention, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

### Dosage Forms

The pharmaceutical composition or combination of the present invention may, for example, be in unit dosage of about 1-1000 mg of active ingredient(s) for a subject of about 50-70 kg.

### Combinations

“Combination” refers to either a fixed combination in one dosage unit form, or a combined administration where a compound of formula (I), or a pharmaceutically acceptable salt thereof, and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one therapeutic agent and includes both fixed and non-fixed combinations of the therapeutic agents. The term “fixed combination” means that the therapeutic agents, e.g. a compound of the present invention and a combination partner, are both administered to a patient simultaneously in the form of a single entity or dosage. The term “non-fixed combination” means that the therapeutic agents, e.g. a compound of the present invention and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more therapeutic agents.

The combinations described herein can include a compound of formula (I) and one or more additional therapeutic agents, e.g., one or more anti-cancer agents, cytotoxic or cytostatic agents, hormone treatment, vaccines, and/or other immunotherapies. In other embodiments, the combination is further administered or used in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications associated with the treatment.

There is also provided a combination comprising a compound of formula (I) as described herein, or a pharmaceutically acceptable salt thereof, as described herein, and one or more additional therapeutically active agents. The additional therapeutic agent is, for example, a chemical compound, peptide, antibody, antibody fragment or nucleic acid, which is therapeutically active or enhances the therapeutic activity when administered to a patient in combination with a compound of the present disclosure. In particular, an additional therapeutically active agent is:
an anti-cancer agent,
a chemotherapy,
a chemotherapy selected from anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®), in particular fluorouracil (5-FU) and irinotecan (Camptosar®).
   a PD-1 inhibitor,
   an anti-PD-1 antibody molecule, or
   a PD-1 inhibitor selected from PDR001 (Novartis), Nivolumab (Bristol-Myers Squibb), Pembrolizumab (Merck & Co), Pidilizumab (CureTech), MEDI0680 (Medimmune), Cemiplimab (REGN2810, Regeneron), Dostarlimab (TSR-042, Tesaro), PF-06801591 (Pfizer), Tislelizumab (BGB-A317, Beigene), BGB-108 (Beigene), INCSHR1210 (Incyte), Balstilimab (AGEN2035, Agenus), Sintilimab (InnoVent), Toripalimab (Shanghai Junshi Bioscience), Camrelizumab (Jiangsu Hengrui Medicine Co.), and AMP-224 (Amplimmune), Penpulimab (Akeso Biopharma Inc), Zimberelimab (Arcus Biosciences Inc) and Prolgolimab (Biocad Ltd), in particular PDR001, more particularly Tislelizumab (BGB-A317, Beigene).

In a further embodiment, the additional therapeutically active agent is the chemotherapy irinotecan (Camptosar®).

In another embodiment, the additional therapeutically active agent is an inhibitor of PD-1, e.g., human PD-1. In another embodiment, the immunomodulator is an inhibitor of PD-L1, e.g., human PD-L1. In one embodiment, the inhibitor of PD-1 or PD-L1 is an antibody molecule to PD-1 or PD-L1. In another embodiment, the additional therapeutically active agent is an anti-PD-1 antibody molecule.

In a further embodiment, the PD-1 inhibitor is an anti-PD-1 antibody molecule as described in US 2015/0210769, published on Jul. 30, 2015, entitled “Antibody Molecules to PD-1 and Uses Thereof,” incorporated by reference in its entirety.

In another embodiment, there is provided a combination of a compound of formula (I) or a pharmaceutically acceptable salt thereof, and a chemotherapy, and a PD-1 inhibitor. In particular, the chemotherapy and PD-1 inhibitor are selected from those described above. More particularly, the chemotherapy is irinotecan (Camptosar®) and the PD-1 inhibitor is PDR001 or Tislelizumab. Tislelizumab can have a heavy chain of SEQ ID NO: 3 and a light chain of SEQ ID NO: 4. In some embodiments, the anti-PD-1 antibody is dosed at 100 mg per week. In some embodiments, tislelizumab and is dosed at 300 mg IV on day 1 of each 28 day cycle. In some embodiments, tislelizumab can be dosed at 500 mg once every four (4) weeks.

In another embodiment, the anti-PD-1 antibody molecule, e.g., tislelizumab, and comprises a heavy chain and/or light chain, VH, VL, HCDR1, HCDR2, HCDR3, LCDR1, LCDR2, and LCDR3 of the following:

| | AMINO ACID SEQUENCE | |
|---|---|---|
| Heavy | QVQLQESGPGLVKPSETLSLTCTVSGFSLTSYGVHWI | SEQ ID NO: 3 |
| Chain | RQPPGKGLEWIGVIYADGSTNYNPSLKSRVTISKDTSK | |
| | NQVSLKLSSVTAADTAVYYCARAYGNYWYIDVWGQG | |
| | TTVTVSSASTKGPSVFPLAPCSRSTSESTAALGCLVKD | |
| | YFPEPVTVSWNSGALTSGVHTFPAVLQSSGLYSLSSV | |
| | VTVPSSSLGTKTYTCNVDHKPSNTKVDKRVESKYGPP | |
| | CPPCPAPPVAGGPSVFLFPPKPKDTLMISRTPEVTCVVV | |
| | VAVSQEDPEVQFNWYVDGVEVHNAKTKPREEQFNST | |
| | YRVVSVLTVVHQDWLNGKEYKCKVSNKGLPSSIEKTIS | |
| | KAKGQPREPQVYTLPPSQEEMTKNQVSLTCLVKGFYP | |
| | SDIAVEWESNGQPENNYKTTPPVLDSDGSFFLYSKLT | |
| | VDKSRWQEGNVFSCSVMHEALHNHYTQKSLSLSLGK | |
| Light | DIVMTQSPDSLAVSLGERATINCKSSESVSNDVAWYQ | SEQ ID NO: 4 |
| Chain | QKPGQPPKLLINYAFHRFTGVPDRFSGSGYGTDFTLTI | |
| | SSLQAEDVAVYYCHQAYSSPYTFGQGTKLEIKRTVAA | |
| | PSVFIFPPSDEQLKSGTASVVCLLNNFYPREAKVQWK | |
| | VDNALQSGNSQESVTEQDSKDSTYSLSSTLTLSKADY | |
| | EKHKVYACEVTHQGLSSPVTKSFNRGEC | |
| Variable | QVQLQESGPGLVKPSETLSLTCTVSGFSLTSYGVHWI | SEQ ID NO: 5 |
| Heavy | RQPPGKGLEWIGVIYADGSTNYNPSLKSRVTISKDTSK | |
| Chain | NQVSLKLSSVTAADTAVYYCARAYGNYWYIDVWGQG | |
| | TTVTVSS | |
| Variable | DIVMTQSPDSLAVSLGERATINCKSSESVSNDVAWYQ | SEQ ID NO: 6 |
| Light | QKPGQPPKLLINYAFHRFTGVPDRFSGSGYGTDFTLTI | |
| Chain | SSLQAEDVAVYYCHQAYSSPYTFGQGTKLEIK | |
| HCDR1 | GFSLTSYGVH | SEQ ID NO: 7 |
| HCDR2 | VIYADGSTNYNPSLKS | SEQ ID NO: 8 |
| HCDR3 | ARAYGNYWYIDV | SEQ ID NO: 9 |
| LCDR1 | KSSESVSNDVA | SEQ ID NO: 10 |
| LCDR2 | YAFHRFT | SEQ ID NO: 11 |
| LCDR3 | HQAYSSPYT | SEQ ID NO: 12 |

In some embodiments, the PD-1 inhibitor comprises the HCDRs and LCDRs of tislelizumab as set forth in SEQ ID NOs: 7-12.

In some embodiments, the PD-1 inhibitor (e.g., tislelizumab) is administered at a flat dose of between about 100 mg to about 600 mg. In some embodiments, the PD-1 inhibitor is administered at a dose of between about 100 mg to about 500 mg. In some embodiments, the PD-1 inhibitor is administered at a dose of between about 100 mg to about 400 mg. In some embodiments, the PD-1 inhibitor is administered at a dose of between about 100 mg to about 300 mg. In some embodiments, the PD-1 inhibitor is administered at a dose of between about 100 mg to about 200 mg. In some embodiments, the PD-1 inhibitor is administered at a dose of between about 200 mg to about 600 mg. In some embodiments, the PD-1 inhibitor is administered at a dose of between about 200 mg to about 500 mg. In some embodiments, the PD-1 inhibitor is administered at a dose of between about 200 mg to about 400 mg. In some embodiments, the PD-1 inhibitor is administered at a dose of between about 200 mg to about 300 mg. In some embodiments, the PD-1 inhibitor is administered at a dose of between about 300 mg to about 600 mg. In some embodiments, the PD-1 inhibitor is administered at a dose of between about 300 mg to about 500 mg. In some embodiments, the PD-1 inhibitor is administered at a dose of between about 300 mg to about 400 mg. In some embodiments, the PD-1 inhibitor is administered at a dose of between about 400 mg to about 600 mg. In some embodiments, the PD-1 inhibitor is administered at a dose of between about 400 mg to about 500 mg. In some embodiments, the PD-1 inhibitor is administered at a dose of between about 500 mg to about 600 mg. In some embodiments, the PD-1 inhibitor is administered at a dose of between about 600 mg to about 700 mg. In some embodiments, the PD-1 inhibitor is administered at a dose of between about 700 mg to about 800 mg. In some embodiments, the PD-1 inhibitor is administered at a dose of between about 800 mg to about 900 mg. In some embodiments, the PD-1 inhibitor is administered at a dose of between about 900 mg to about 1000 mg.

In some embodiments, the PD-1 inhibitor (e.g., tislelizumab) is administered at a flat dose of about 100 mg. In some embodiments, the PD-1 inhibitor is administered at a dose of about 200 mg. In some embodiments, the PD-1 inhibitor is administered at a dose of about 300 mg. In some embodiments, the PD-1 inhibitor is administered at a dose of about 400 mg. In some embodiments, the PD-1 inhibitor is administered at a dose of about 500 mg. In some embodiments, the PD-1 inhibitor is administered at a dose of about 600 mg. In some embodiments, the PD-1 inhibitor is administered at a dose of about 700 mg. In some embodiments, the PD-1 inhibitor is administered at a dose of about 800 mg. In some embodiments, the PD-1 inhibitor is administered at a dose of about 900 mg. In some embodiments, the PD-1 inhibitor is administered at a dose of about 1000 mg.

In some embodiments, the PD-1 inhibitor (e.g., tislelizumab) is administered once every ten weeks. In some embodiments, the PD-1 inhibitor is administered once every nine weeks. In some embodiments, the PD-1 inhibitor is administered once every eight weeks. In some embodiments, the PD-1 inhibitor is administered once every seven weeks. In some embodiments, the PD-1 inhibitor is administered once every six weeks. In some embodiments, the PD-1 inhibitor is administered once every five weeks. In some embodiments, the PD-1 inhibitor is administered once every four weeks. In some embodiments, the PD-1 inhibitor is administered once every three weeks. In some embodiments, the PD-1 inhibitor is administered once every two weeks. In some embodiments, the PD-1 inhibitor is administered once every week.

In some embodiments, the PD-1 inhibitor (e.g., tislelizumab) is administered intravenously.

In some embodiments, the PD-1 inhibitor (e.g., tislelizumab) is administered over a period of about 20 minutes to 40 minutes (e.g., about 30 minutes). In some embodiments, the PD-1 inhibitor is administered over a period of about 30 minutes. In some embodiments, the PD-1 inhibitor is administered over a period of about an hour. In some embodiments, the PD-1 inhibitor is administered over a period of about two hours. In some embodiments, the PD-1 inhibitor is administered over a period of about three hours. In some embodiments, the PD-1 inhibitor is administered over a period of about four hours. In some embodiments, the PD-1 inhibitor is administered over a period of about five hours. In some embodiments, the PD-1 inhibitor is administered over a period of about six hours.

In some embodiments, the PD-1 inhibitor (e.g., tislelizumab) is administered at a dose between about 300 mg to about 500 mg (e.g., about 400 mg), intravenously, once every four weeks. In some embodiments, the PD-1 inhibitor is administered at a dose between about 200 mg to about 400 mg (e.g., about 300 mg), intravenously, once every three weeks. In some embodiments, tislelizumab is administered at a dose of 400 mg, once every four weeks. In some embodiments, tislelizumab is administered at a dose of 300 mg, once every three weeks.

In some embodiments, the PD-1 inhibitor (e.g., tislelizumab) is administered at a dose between about 300 mg to about 500 mg (e.g., about 400 mg), intravenously, over a period of about 20 minutes to about 40 minutes (e.g., about 30 minutes), once every two weeks. In some embodiments, the PD-1 inhibitor is administered at a dose between about 200 mg to about 400 mg (e.g., about 300 mg), intravenously, over a period of about 20 minutes to about 40 minutes (e.g., about 30 minutes), once every three weeks.

In some embodiments, the PD-1 inhibitor (e.g., tislelizumab) is administered at a dose of about 100 mg per week. For example, if a 10-week dose is given to a patient, then the PD-1 inhibitor (e.g., tislelizumab) can be given at 1000 mg. If a 9-week dose is given, then the PD-1 inhibitor (e.g., tislelizumab) can be given at 900 mg. If an 8-week dose is given, then the PD-1 inhibitor (e.g., tislelizumab) can be given at 800 mg. If a 7-week dose is given, then the PD-1 inhibitor (e.g., tislelizumab) can be given at 700 mg. If a 6-week dose is given, then the PD-1 inhibitor (e.g., tislelizumab) can be given at 600 mg. If a 5-week dose is given, then the PD-1 inhibitor (e.g., tislelizumab) can be given at 500 mg. If a 4-week dose is given, then the PD-1 inhibitor (e.g., tislelizumab) can be given at 400 mg. If a 3-week dose is given, then the PD-1 inhibitor (e.g., tislelizumab) can be given at 300 mg. If a 2-week dose is given, then the PD-1 inhibitor (e.g., tislelizumab) can be given at 200 mg. If a 1-week dose is given, then the PD-1 inhibitor (e.g., tislelizumab) can be given at 100 mg.

For example, if an anti-PD-1 antibody, such as tislelizumab is used, it can be administered at a dose of 200 mg as an intravenous infusion, once every three week. Alternatively, tislelizumab can be administered at a dose of 300 mg as an intravenous infusion, once every four weeks. If an anti-PD-1 antibody, such as tislelizumab is used, it can be administered at a dose of 300 mg as an intravenous infusion, once every three week. Alternatively, tislelizumab can be administered at a dose of 400 mg as an intravenous infusion, once every four weeks.

The structure of the active compounds identified by code numbers, generic or trade names may be taken from the actual edition of the standard compendium “The Merck Index” or from databases, e.g. Patents International (e.g. IMS World Publications). The above-mentioned compounds, which can be used in combination with a compound of the present invention, can be prepared and administered as described in the art, such as in the documents cited above.

In one embodiment, the invention provides a product comprising a compound of formula (I) of the present invention and at least one other therapeutic agent as a combined preparation for simultaneous, separate or sequential use in therapy. In one embodiment, the therapy is the treatment of a disease or condition mediated by WRN. Products provided as a combined preparation include a composition comprising the compound of formula (I) and the other therapeutic agent(s) together in the same pharmaceutical composition, or the compound of the present invention and the other therapeutic agent(s) in separate form, e.g. in the form of a kit.

In one embodiment, the invention provides a kit comprising two or more separate pharmaceutical compositions, at least one of which contains a compound of formula (I) of the present invention. In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

In the combination therapies of the invention, the compound of formula (I) of the present invention and the other therapeutic agent may be manufactured and/or formulated by the same or different manufacturers. Moreover, the compound of the present invention and the other therapeutic may be brought together into a combination therapy: (i) prior to release of the combination product to physicians (e.g. in the case of a kit comprising the compound of the present invention and the other therapeutic agent); (ii) by the physician themselves (or under the guidance of the physician) shortly before administration; (iii) in the patient themselves, e.g. during sequential administration of the compound of the present invention and the other therapeutic agent.

Accordingly, the invention provides the use of a compound of formula (I) of the present invention for treating a disease or condition mediated by WRN, wherein the medicament is prepared for administration with another therapeutic agent. The invention also provides the use of another therapeutic agent for treating a disease or condition mediated by WRN, wherein the medicament is administered with a compound of formula (I) of the present invention.

The invention also provides a compound of formula (I) of the present invention for use in treating a disease or condition mediated by WRN, wherein the compound of formula (I) of the present invention is prepared for administration with another therapeutic agent. The invention also provides another therapeutic agent for use in treating a disease or condition mediated by WRN, wherein the other therapeutic agent is prepared for administration with a compound of the present invention. The invention also provides a compound of formula (I) of the present invention for use in treating a disease or condition mediated by WRN, wherein the compound of the present invention is administered with another therapeutic agent. The invention also provides another therapeutic agent for use in a method of treating a disease or condition mediated by WRN, wherein the other therapeutic agent is administered with a compound of formula (I) of the present invention.

The invention also provides the use of a compound of formula (I) of the present invention for treating a disease or condition mediated by WRN, wherein the patient has previously (e.g. within 24 hours) been treated with another therapeutic agent. The invention also provides the use of another therapeutic agent for treating a disease or condition mediated by WRN, wherein the patient has previously (e.g. within 24 hours) been treated with compound of the present invention.

### Biological Assays and Data

The activity of a compound according to the present invention can be assessed by the following in vitro & in vivo methods.

### Material and Methods

Molecular Biology and virus production. The DNA encoding human Werner helicase (UniProt Q14191, WRN, amino acids S2-S1432) was designed as four DNA strings which were codon-optimized for expression in E. coli. The strings were either ordered from GeneArt (LifeTechnologies, Regensburg, Germany) or made with subcloning overlapping oligonucleotides.

The baculovirus from expression plasmid pLAF1202 (SEQ ID NO: 1) encoding His-ZZ-3C-WRN (aa N517-P1238, encoded by nucleotides 578-2743 in the sequence) was generated with the FlashBac Ultra system (Oxford Expression Technologies 100302) using 540 ng of plasmid DNA, 5.4 μg Flashbac Ultra DNA, and 5.4 microliters Lipofectin (LifeTechnologies 18292-011) for transfection following the manufacturer's instructions. After 5 hours incubation the solution was diluted with 500 microliters TC100 medium (LifeTechnologies 13055-025) and incubated for 7 days at 27° C.

The cells were harvested by centrifugation at 800×g for 10 minutes and the supernatant containing the virus was transferred into a new sterile tube. For the first virus amplification, 500 microliters of the virus was added to 25 mL of SF9 cells at one million cells/mL and incubated for 5 days at 27° C. (200 rpm). The cell viability, density, and diameter was measured and the virus, upon signs of infection, was harvested by centrifugation at 3000 rpm for 15 minutes.

Baculovirus infected insect cells (BIICs) were generated as described by Wasilko et al., 2009, DOI: 10.1016/j.pep.2009.01.002.

In brief, in an Erlenmeyer flask 100 million SF9 cells (one million cells/mL) in 100 mL ESF921 medium (Expression Systems—96-001-01, supplemented with 0.5× Streptomycin/Penicillin) were infected with 300 million baculovirus particles of the respective construct (estimated MOI=3) and incubated at 27° C. for 24 hours at 130 rpm. The infected cells were transferred to 50 mL tubes and harvested by centrifugation at 100×g for 10 minutes at RT.

The cells were resuspended to 10 million/mL in ESF921 (0.5× Streptomycin/Penicillin) medium with BSA (final 10 mg/mL) and 10% DMSO. 500 μL aliquots of cells were transferred to 1.8 mL cryotubes and frozen in Nunc Cryo 1° C. freezing container overnight at −80° C.

SEQ ID NO: 1
AACCATCTCGCAAATAAATAAGTATTTTACTGTTTTCGTAACAGTTTTGTAATAAAAAAAC
CTATAAATATTCCGGATTATTCATACCGTCCCACCATCGGGCGCCATGGCTTCTCACCA
CCATCACCATCACCATCATCATCACGCTCAGCACGACGAGGCTGTGGACAACAAGTTC
AACAAGGAGCAGCAGAACGCTTTCTACGAGATCCTGCACCTCCCTAACCTGAACGAGG
AGCAGCGTAACGCTTTCATCCAGTCCCTGAAGGACGACCCTTCTCAGTCTGCTAACCTG
CTGGCTGAGGCTAAGAAGCTGAACGACGCTCAGGCTCCTAAGGTGGACAACAAGTTCA
ACAAGGAGCAGCAGAACGCTTTCTACGAGATCCTGCACCTCCCTAACCTGAACGAGGA
GCAGCGTAACGCTTTCATCCAGTCCCTGAAGGACGACCCTTCTCAGTCTGCTAACCTG
CTGGCTGAGGCTAAGAAGCTGAACGACGCTCAGGCTCCTAAGGTGGACGCTAACGGT
GGCGGCGGTTCCGGCGGTGGTGGCTCTCTCGAGGTTCTGTTCCAGGGTCCGAATGAA
GGCGAGGAAGATGATGACAAAGACTTCCTGTGGCCAGCTCCAAACGAAGAACAGGTGA
CTTGTCTCAAGATGTACTTCGGTCATAGCAGCTTCAAACCAGTGCAGTGGAAAGTTATC
CACAGCGTTCTTGAAGAACGTCGTGATAATGTGGCTGTGATGGCTACTGGCTATGGTAA
GAGCCTGTGTTTCCAGTACCCGCCAGTTTACGTTGGTAAGATCGGTCTGGTGATTAGCC
CGCTGATCTCTCTGATGGAAGACCAGGTGCTGCAACTTAAGATGAGCAACATCCCGGC
TTGTTTCCTGGGTTCTGCACAAAGCGAGAACGTGCTCACCGATATCAAGCTGGGTAAGT
ACCGTATCGTGTACGTGACGCCAGAATACTGTAGCGGCAACATGGGTCTTCTGCAACA
GCTCGAAGCTGATATTGGCATCACCCTCATTGCAGTGGACGAAGCTCACTGTATCAGC
GAGTGGGGTCATGATTTCCGCGACTCTTTCCGTAAACTGGGTTCTCTGAAGACTGCACT
TCCGATGGTTCCAATTGTGGCACTGACCGCAACTGCTTCTAGCTCTATTCGTGAAGACA
TCGTTCGTTGCCTGAACCTCCGTAACCCACAAATTACCTGCACCGGCTTTGACCGTCCG
AACCTGTACCTGGAGGTTCGTCGTAAGACCGGTAATATCCTTCAGGACCTGCAACCATT
CCTGGTTAAGACCAGCAGCCACTGGGAGTTCGAAGGTCCGACTATCATCTACTGCCCA
AGCCGTAAGATGACCCAGCAGGTTACTGGTGAACTGCGTAAACTGAACCTGAGCTGTG
GCACTTACCACGCAGGCATGTCTTTCTCTACCCGTAAAGACATCCATCATCGTTTCGTG
CGTGATGAAATCCAGTGCGTTATCGCTACCATTGCATTCGGCATGGGTATCAACAAAGC
TGACATCCGTCAAGTGATTCACTACGGTGCACCGAAAGACATGGAAAGCTACTACCAG
GAAATCGGCCGTGCAGGTCGTGATGGTCTGCAAAGCTCTTGTCATGTGCTGTGGGCAC
CAGCAGATATTAACCTGAACCGTCACCTGCTGACTGAAATTCGTAACGAGAAATTCCGT
CTGTACAAACTGAAGATGATGGCGAAGATGGAGAAATACCTGCATAGCTCCCGTTGTC
GTCGTCAAATCATTCTGAGCCATTTCGAGGATAAACAGGTGCAGAAAGCTTCTCTGGGT
ATCATGGGCACTGAGAAGTGCTGCGATAACTGTCGTAGCCGTCTTGATCACTGCTACA
GCATGGACGATAGCGAAGACACTTCTTGGGATTTCGGTCCACAAGCATTCAAACTGCTG
AGCGCAGTTGATATCCTGGGTGAGAAATTCGGCATCGGCCTCCCAATCCTGTTTCTGC
GCGGTTCTAACTCTCAGCGTCTTGCTGATCAATACCGTCGTCACTCTCTGTTCGGCACT
GGTAAAGACCAGACCGAATCTTGGTGGAAAGCATTCAGCCGTCAACTTATCACCGAAG
GCTTTCTGGTGGAAGTGTCTCGTTACAACAAGTTCATGAAGATCTGCGCACTGACTAAG
AAAGGTCGTAACTGGCTGCACAAGGCAAATACCGAGTCTCAGTCTCTTATCCTTCAGGC
TAACGAAGAACTGTGCCCGAAGAAGCTTCTGCTGCCATCTTCTAAGACCGTGAGCTCTG
GTACTAAAGAGCATTGCTACAACCAGGTGCCGGTTGAACTGTCTACCGAGAAGAAGTC
CAACCTGGAGAAGCTGTACTCCTACAAACCGTGCGACAAGATCTCCTCCGGTTCTAATA
TCAGCAAGAAGTCCATCATGGTGCAGTCTCCGGAGAAAGCTTACAGCAGCTCTCAGCC
AGTTATCTCTGCACAGGAACAGGAAACTCAGATTGTGCTGTACGGTAAACTGGTGGAAG
CACGTCAGAAACACGCTAACAAGATGGACGTGCCGCCAGCAATTCTTGCAACCAACAA
GATTCTGGTGGACATGGCTAAGATGCGCCCAACTACTGTTGAGAACGTGAAACGTATC
GACGGTGTTAGCGAAGGTAAAGCTGCAATGCTGGCACCACTGCTTGAAGTTATCAAGC
ATTTCTGCCAGACCAACTCTGTTCAGACCGACCTGTTCTCTTCTACCAAACCATAATGGT
ACCGAATTCGCGGCCGCAGAGCTCGCTCTGGTGCCACGCGGTAGTTCCGCTTGGAGC
CACCCGCAGTTCGAAAAGTAAGTGATTAACCTCAGGTTATACATATATTTTGAATTTAAT
TAATTATACATATATTTTATATTATTTTTGTCTTTTATTATCGAGGGGCCGTTGTTGGTGT
GGGGTTTTGCATAGAAATAACAATGGGAGTTGGCGACGTTGCTGCGCCAACACCACCT
CCCTTCCCTCCTTTCATCATGTATCTGTAGATAAAATAAAATATTAAACCTAAAAACAAGA
CCGCGCCTATCAACAAAATGATAGGCATTAACTTGCCGCTGACGCTGTCACTAACGTTG
GACGATTTGCCGACTAAACCTTCATCGCCCAGTAACCAATCTAGGTAGCTGAGCGCATG
CAAGCTGATCCGGGTTATTAGTACATTTATTAAGCGCTAGATTCTGTGCGTTGTTGATTT
ACAGACAATTGTTGTACGTATTTTAATAATTCATTAAATTTATAATCTTTAGGGTGGTATG
TTAGAGCGAAAATCAAATGATTTTCAGCGTCTTTATATCTGAATTTAAATATTAAATCCTC
AATAGATTTGTAAAATAGGTTTCGATTAGTTTCAAACAAGGGTTGTTTTTCCGAACCGAT
GGCTGGACTATCTAATGGATTTTCGCTCAACGCCACAAAACTTGCCAAATCTTGTAGCA
GCAATCTAGCTTTGTCGATATTCGTTTGTGTTTTGTTTTGTAATAAAGGTTCGACGTCGT
TCAAAATATTATGCGCTTTTGTATTTCTTTCATCACTGTCGTTAGTGTACAATTGACTCGA
CGTAAACACGTTAAATAGAGCTTGGACATATTTAACATCGGGCGTGTTAGCTTTATTAGG
CCGATTATCGTCGTCGTCCCAACCCTCGTCGTTAGAAGTTGCTTCCGAAGACGATTTTG
CCATAGCCACACGACGCCTATTAATTGTGTCGGCTAACACGTCCGCGATCAAATTTGTA
GTTGAGCTTTTTGGAATTATTTCTGATTGCGGGCGTTTTTGGGCGGGTTTCAATCTAACT
GTGCCCGATTTTAATTCAGACAACACGTTAGAAAGCGATGGTGCAGGCGGTGGTAACA
TTTCAGACGGCAAATCTACTAATGGCGGCGGTGGTGGAGCTGATGATAAATCTACCATC
GGTGGAGGCGCAGGCGGGGCTGGCGGCGGAGGCGGAGGCGGAGGTGGTGGCGGTG
ATGCAGACGGCGGTTTAGGCTCAAATGTCTCTTTAGGCAACACAGTCGGCACCTCAACT
ATTGTACTGGTTTCGGGCGCCGTTTTTGGTTTGACCGGTCTAAGACGAGTGCGATTTTT
TTCGTTTCTAATAGCTTCCAACAATTGTTGTCTGTCGTCTAAAGGTGCAGCGGGTTGAG
GTTCCGTCGGCATTGGTGGAGCGGGCGGCAATTCAGACATCGATGGTGGTGGTGGTG
GTGGAGGCGCTGGAATGTTAGGCACGGGAGAAGGTGGTGGCGGCGGTGCCGCCGGT
ATAATTTGTTCTGGTTTAGTTTGTTCGCGCACGATTGTGGGCACCGGCGCAGGCGCCG
CTGGCTGCACAACGGAAGGTCGTCTGCTTCGAGGCAGCGCTTGGGGTGGTGGCAATT
CAATATTATAATTGGAATACAAATCGTAAAAATCTGCTATAAGCATTGTAATTTCGCTATC
GTTTACCGTGCCGATATTTAACAACCGCTCAATGTAAGCAATTGTATTGTAAAGAGATTG
TCTCAAGCTCGGATCGATCCCGCACGCCGATAACAAGCCTTTTCATTTTTACTACAGCA
TTGTAGTGGCGAGACACTTCGCTGTCGTCGAGGTTTAAACGCTTCCTCGCTCACTGACT
CGCTGCGCTCGGTCGTTCGGCTGCGGCGAGCGGTATCAGCTCACTCAAAGGCGGTAA
TACGGTTATCCACAGAATCAGGGGATAACGCAGGAAAGAACATGTGAGCAAAAGGCCA
GCAAAAGGCCAGGAACCGTAAAAAGGCCGCGTTGCTGGCGTTTTTCCATAGGCTCCGC
CCCCCTGACGAGCATCACAAAAATCGACGCTCAAGTCAGAGGTGGCGAAACCCGACAG
GACTATAAAGATACCAGGCGTTTCCCCCTGGAAGCTCCCTCGTGCGCTCTCCTGTTCC
GACCCTGCCGCTTACCGGATACCTGTCCGCCTTTCTCCCTTCGGGAAGCGTGGCGCTT
TCTCATAGCTCACGCTGTAGGTATCTCAGTTCGGTGTAGGTCGTTCGCTCCAAGCTGG
GCTGTGTGCACGAACCCCCCGTTCAGCCCGACCGCTGCGCCTTATCCGGTAACTATCG
TCTTGAGTCCAACCCGGTAAGACACGACTTATCGCCACTGGCAGCAGCCACTGGTAAC
AGGATTAGCAGAGCGAGGTATGTAGGCGGTGCTACAGAGTTCTTGAAGTGGTGGCCTA
ACTACGGCTACACTAGAAGGACAGTATTTGGTATCTGCGCTCTGCTGAAGCCAGTTACC
TTCGGAAAAAGAGTTGGTAGCTCTTGATCCGGCAAACAAACCACCGCTGGTAGCGGTG
GTTTTTTTGTTTGCAAGCAGCAGATTACGCGCAGAAAAAAAGGATCTCAAGAAGATCCT
TTGATCTTTTCTACGGGGTCTGACGCTCAGTGGAACGAAAACTCACGTTAAGGGATTTT
GGTCATGAGATTATCAAAAAGGATCTTCACCTAGATCCTTTTAAATTAAAAATGAAGTTTT
AAATCAATCTAAAGTATATATGAGTAAACTTGGTCTGACAGTTACCAATGCTTAATCAGT
GAGGCACCTATCTCAGCGATCTGTCTATTTCGTTCATCCATAGTTGCCTGACTCCCCGT
CGTGTAGATAACTACGATACGGGAGGGCTTACCATCTGGCCCCAGTGCTGCAATGATA
CCGCGAGACCCACGCTCACCGGCTCCAGATTTATCAGCAATAAACCAGCCAGCCGGAA
GGGCCGAGCGCAGAAGTGGTCCTGCAACTTTATCCGCCTCCATCCAGTCTATTAATTGT
TGCCGGGAAGCTAGAGTAAGTAGTTCGCCAGTTAATAGTTTGCGCAACGTTGTTGCCAT
TGCTACAGGCATCGTGGTGTCACGCTCGTCGTTTGGTATGGCTTCATTCAGCTCCGGTT
CCCAACGATCAAGGCGAGTTACATGATCCCCCATGTTGTGCAAAAAAGCGGTTAGCTC
CTTCGGTCCTCCGATCGTTGTCAGAAGTAAGTTGGCCGCAGTGTTATCACTCATGGTTA
TGGCAGCACTGCATAATTCTCTTACTGTCATGCCATCCGTAAGATGCTTTTCTGTGACTG
GTGAGTACTCAACCAAGTCATTCTGAGAATAGTGTATGCGGCGACCGAGTTGCTCTTGC
CCGGCGTCAATACGGGATAATACCGCGCCACATAGCAGAACTTTAAAAGTGCTCATCAT
TGGAAAACGTTCTTCGGGGCGAAAACTCTCAAGGATCTTACCGCTGTTGAGATCCAGTT
CGATGTAACCCACTCGTGCACCCAACTGATCTTCAGCATCTTTTACTTTCACCAGCGTTT
CTGGGTGAGCAAAAACAGGAAGGCAAAATGCCGCAAAAAAGGGAATAAGGGCGACAC
GGAAATGTTGAATACTCATACTCTTCCTTTTTCAATATTATTGAAGCATTTATCAGGGTTA
TTGTCTCATGAGCGGATACATATTTGAATGTATTTAGAAAAATAAACAAATAGGGGTTCC
GCGCACATTTCCCCGAAAAGTGCCACCTGACGCGCCCTGTAGCGGCGCATTAAGCGC
GGCGGGTGTGGTGGTTACGCGCAGCGTGACCGCTACACTTGCCAGCGCCCTAGCGCC
CGCTCCTTTCGCTTTCTTCCCTTCCTTTCTCGCCACGTTCGCCGGCTTTCCCCGTCAAG
CTCTAAATCGGGGGCTCCCTTTAGGGTTCCGATTTAGTGCTTTACGGCACCTCGACCCC
AAAAAACTTGATTAGGGTGATGGTTCACGTAGTGGGCCATCGCCCTGATAGACGGTTTT
TCGCCCTTTGACGTTGGAGTCCACGTTCTTTAATAGTGGACTCTTGTTCCAAACTGGAA
CAACACTCAACCCTATCTCGGTCTATTCTTTTGATTTATAAGGGATTTTGCCGATTTCGG
CCTATTGGTTAAAAAATGAGCTGATTTAACAAAAATTTAACGCGAATTTTAACAAAATATT
AACGTTTACAATTTCCCATTCGCCATTCAGGCTGCGCAACTGTTGGGAAGGGCGATCG
GTGCGGGCCTCTTCGCTATTACGCCAGGAACGGCTCCGCCCACTATTAATGAAATTAAA
AATTCCAATTTTAAAAAACGCAGCAAGAGAAACATTTGTATGAAAGAATGCGTAGAAGGA
AAGAAAAATGTCGTCGACATGCTGAACAACAAGATTAATATGCCTCCGTGTATAAAAAAA
ATATTGAACGATTTGAAAGAAAACAATGTACCGCGCGGCGGTATGTACAGGAAGAGGTT
TATACTAAACTGTTACATTGCAAACGTGGTTTCGTGTGCCAAGTGTGAAAACCGATGTTT
AATCAAGGCTCTGACGCATTTCTACAACCACGACTCCAAGTGTGTGGGTGAAGTCATGC
ATCTTTTAATCAAATCCCAAGATGTGTATAAACCACCAAACTGCCAAAAAATGAAAACTG
TCGACAAGCTCTGTCCGTTTGCTGGCAACTGCAAGGGTCTCAATCCTATTTGTAATTATT
GAATAATAAAACAATTATAAATGCTAAATTTGTTTTTTATTAACGATACAAACCAAACGCA
ACAAGAACATTTGTAGTATTATCTATAATTGAAAACGCGTAGTTATAATCGCTGAGGTAA
TATTTAAAATCATTTTCAAATGATTCACAGTTAATTTGCGACAATATAATTTTATTTTCACA
TAAACTAGACGCCTTGTCGTCTTCTTCTTCGTATTCCTTCTCTTTTTCATTTTTCTCTTCA
TAAAAATTAACATAGTTATTATCGTATCCATATATGTATCTATCGTATAGAGTAAATTTTTT
GTTGTCATAAATATATATGTCTTTTTTAATGGGGTGTATAGTACCGCTGCGCATAGTTTTT
CTGTAATTTACAACAGTGCTATTTTCTGGTAGTTCTTCGGAGTGTGTTGCTTTAATTATTA
AATTTATATAATCAATGAATTTGGGATCGTCGGTTTTGTACAATATGTTGCCGGCATAGT
ACGCAGCTTCTTCTAGTTCAATTACACCATTTTTTAGCAGCACCGGATTAACATAACTTT
CCAAAATGTTGTACGAACCGTTAAACAAAAACAGTTCACCTCCCTTTTCTATACTATTGT
CTGCGAGCAGTTGTTTGTTGTTAAAAATAACAGCCATTGTAATAAGACGCACAAACTAAT
ATCACAAACTGGAAATGTCTATCAATATATAGTTGCTGATCAGATCTACCCGTAGTGGCT
ATGGCAGGGCTTGCCGCCCCGACGTTGGCTGCGAGCCCTGGGCCTTCACCCGAACTT
GGGGGTTGGGGTGGGGAAAAGGAAGAAACGCGGGCGTATTGGTCCCAATGGGGTCTC
GGTGGGGTATCGACAGAGTGCCAGCCCTGGGACCGAACCCCGCGTTTATGAACAAAC
GACCCAACACCCGTGCGTTTTATTCTGTCTTTTTATTGCCGTCATAGCGCGGGTTCCTT
CCGGTATTGTCTCCTTCCGTGTTTCAGTTAGCCTCCCCCATCTCCCGGTACCGCATGCT
ATGCATCGGCCGCTTTACTTGTACAGCTCGTCCATGCCGAGAGTGATCCCGGCGGCGG
TCACGAACTCCAGCAGGACCATGTGATCGCGCTTCTCGTTGGGGTCTTTGCTCAGGGC
GGACTGGGTGCTCAGGTAGTGGTTGTCGGGCAGCAGCACGGGGCCGTCGCCGATGG
GGGTGTTCTGCTGGTAGTGGTCGGCGAGCTGCACGCTGCCGTCCTCGATGTTGTGGC
GGATCTTGAAGTTCACCTTGATGCCGTTCTTCTGCTTGTCGGCCATGATATAGACGTTG
TGGCTGTTGTAGTTGTACTCCAGCTTGTGCCCCAGGATGTTGCCGTCCTCCTTGAAGTC
GATGCCCTTCAGCTCGATGCGGTTCACCAGGGTGTCGCCCTCGAACTTCACCTCGGCG
CGGGTCTTGTAGTTGCCGTCGTCCTTGAAGAAGATGGTGCGCTCCTGGACGTAGCCTT
CGGGCATGGCGGACTTGAAGAAGTCGTGCTGCTTCATGTGGTCGGGGTAGCGGCTGA
AGCACTGCACGCCGTAGGTCAGGGTGGTCACGAGGGTGGGCCAGGGCACGGGCAGC
TTGCCGGTGGTGCAGATGAACTTCAGGGTCAGCTTGCCGTAGGTGGCATCGCCCTCGC
CCTCGCCGGACACGCTGAACTTGTGGCCGTTTACGTCGCCGTCCAGCTCGACCAGGAT
GGGCACCACCCCGGTGAACAGCTCCTCGCCCTTGCTCACCATCGTCGAGATCCCGGG
CGTTTAAATTGTGTAATTTATGTAGCTGTAATTTTTACCTTATTAATATTTTTTACGCTTTG
CATTCGACGACTGAACTCCCAAATATATGTTTAACTCGTCTTGGTCGTTTGAATTTTTGT
TGCTGTGTTTCCTAATATTTTCCATCACCTTAAATATGTTATTGTAATCCTCAATGTTGAA
CTTGCAATTGGACACGGCATAGTTTTCCATAGTCGTGTAAAACATGGTATTGGCTGCAT
TGTAATACATCCGACTGAGCGGGTACGGATCTATGTGTTTGAGCAGCCTGTTCAAAAAC
TCTGCATCGTCGCAAAACGGAATTTGGTACCCGGGCGTATACTCCGGAATATTAATAGA
TCATGGAGATAATTAAAATGAT

### Protein Expression and Purification

BIICs aliquots for Werner helicase protein His-ZZ-3C-WRN (aa N517-P1238, pLAF1202) were diluted 1/100 into ESF921 medium and further diluted 1/100 into the expression/production flasks with Sf21 cells (one million cells/mL) in 1 L ESF921 medium and incubated for protein expression for 96 h (27° C., 130 rpm).

The WRN protein was purified using the following protocol. The cell pellets were thawed and resuspended in 80 mL buffer A (50 mM Tris, 300 mM NaCl, 20 mM imidazole, 1 mM TCEP, 10% glycerol, pH 7.8) supplemented with Turbonuclease (final concentration 40 units/mL, Merck) and cOmplete protease inhibitor tablets (1 tablet/50 mL, Roche). The cells were lysed by three passages through a homogenizer (Avestin, Emulsiflex C₃) at 800-1000 bar. The lysed sample was centrifuged at 48000×g for 40 minutes (Sorvall RC5B, SS-34 rotor) and the supernatant was passed through a 0.45 μm filter.

The lysate was loaded onto a HisTrap crude FF 5 mL column (GE Healthcare) mounted on an AKTA Pure 25 chromatography system (GE Healthcare). Contaminating proteins were washed away with buffer A and bound protein was eluted with a linear gradient over 10 column volumes to 100% of buffer B (50 mM Tris, 300 mM NaCl, 300 mM imidazole, 1 mM TCEP, 10% glycerol, pH 7.8). 1% (w/w) HRV 3C protease (His-MBP-tagged, produced in-house) was added to the eluted protein. The N-terminal purification tag was cleaved off by the protease during dialysis overnight at 5° C. against 2 L buffer (50 mM Tris pH 7.0, 150 mM NaCl, 1 mM TCEP, 10% glycerol, 0.02% CHAPS). The protein solution was then carefully diluted with adding two volume parts of 20 mM Tris pH 7.0, 10% glycerol, 0.02% CHAPS. The slightly turbid protein solution was passed over a 0.45 μm filter. The cleaved protein was loaded onto a Resource S 6 mL column (GE Healthcare) pre-equilibrated with 20 mM Tris, 20 mM NaCl, 1 mM TCEP, 10% glycerol, pH 7.0. Cleaved tag and contaminating proteins were washed away with the equilibration buffer. The bound target protein was eluted with a linear gradient over 20 column volumes of the same buffer containing 1 M sodium chloride and then injected onto a HiLoad 16/600 Superdex 75 μg column (GE Healthcare) pre-equilibrated with 50 mM Tris pH 7.4, 300 mM NaCl, 10% glycerol. Fractions containing pure protein were identified by SDS-PAGE and pooled. The purified protein was finally split into aliquots and frozen on dry ice. The purity, quantity, and identity of the protein was determined by RP-HPLC and LC-MS.

### In Vitro Enzymatic Activity Assay on WRN Helicase

An ATPase assay was set up to measure the DNA dependent ATP hydrolysis activity of WRN helicase. This assay was used also to assess the inhibition properties of compounds of the invention on DNA dependent WRN ATPase activity.

The core helicase motif of the WRN protein (aa N517-P1238) was produced for this assay (protein production as described above). A 45 oligonucleotide sequence called “FLAP26” as described by Brosh et al., 2009, DOI: 10.1074/jbc.M111446200 (TTTTTTTTTTTTTTTTTTTTTTCCAAGTAAAACGACGGCCAGTGC; SEQ ID NO: 2) was purchased from IDT (Integrated DNA Technologies, Leuven, Belgium) and used as single strand DNA substrate. The ADP-Glo assay kit (Promega, Madison, WI) allowing the quantification of ADP produced in ATP hydrolysis reactions was used for setting up this assay. Time course experiments were first performed in order to determine the best enzymatic assay conditions (including buffer conditions, reaction time and concentrations of protein, ATP and DNA substrates). A typical reaction consists of 10 nM WRN protein, 0.2 nM FLAP26, and 300 micromolar ATP in the following assay buffer: 30 mM Tris pH7.5, 2 mM MgCl₂, 0.02% BSA, 50 mM NaCl, 0.1% pluronic F127 prepared in DNAse free water.

To evaluate the inhibition properties of compounds of the invention, serial dilutions were prepared in DMSO (10 half log dilutions from a 10 mM DMSO solution). 50 nanoliters of each concentration was pre-incubated for 3 hours in a 384 small volume assay plate (Greiner #784075) with 2.5 microliters of a 20 nM WRN helicase protein in assay buffer with 600 micromolar ATP. Control wells were included with a “high control” (no inhibition), containing DMSO with no test compound, and “low controls” (maximal inhibition), containing buffer without protein. The reaction was started by addition of 2.5 microliters of FLAP26 at 0.4 nM and incubated for 30 minutes at room temperature. The reaction was stopped with the addition of 5 microliters of the first ADP-Glo reagent and incubated for one hour to remove the excess amount of ATP. Afterwards, 10 microliters of ATP detection reagent was added and incubated for an additional hour before reading. Luminescence output was recorded using Tecan 1000 reader, with 5 minutes delay before reading. Each concentration of compound was tested in duplicates in the assay plate.

Data analysis was carried out using an in-house developed software (Novartis Helios software application, Novartis Institutes for BioMedical Research, unpublished) using the methods described by Formenko et al., 2006, DOI: 10.1016/j.cmpb.2006.01.008. Following normalization of activity values for the wells to % inhibition (% inhibition=[(high control-sample)/(high control-low control)]×100), IC₅₀ fitting was carried out from the duplicate determinations present on each plate according to [4]. Data analysis can also be carried out using commercially available software designed to derive IC₅₀ values using 4-parameter fits (e.g. GraphPad Prism, XL fit). The reported IC₅₀ values are the geometrical means of at least 2 independent replicates.

### Method for Detecting Effects on Cellular Proliferation

The colon carcinoma cell lines SW48 (RRID: CVCL_1724), HCT 116 (RRID: CVCL_0291) and SNU-407 (RRID: CVCL_5058) were obtained from ATCC. The WRN-knockdown insensitive colon carcinoma cell line DLD-1 (RRID: CVCL_0248) was obtained from the Korean Cell Line Bank (KCLB), and used to generate a derivative in which the endogenous WRN gene copies were knocked out by CRISPR-mediated editing using standard CRISPR methods. The resulting cell line, DLD1-WRN-KO, was used to assess potential off-target compound effects.

SW48, SNU-407 and DLD1-WRN-KO cells were cultured in growth medium composed of RPMI-1640 (Amimed Cat #1-41F22-1), 2 mM L-Glutamine (Amimed Cat #5-10K50), 10 mM HEPES (Gibco Cat #15630-056), 1 mM sodium pyruvate (Amimed Cat #5-60F00-H), 1× Penicillin-Streptomycin (Amimed Cat #4-01F00-H) and 10% fetal calf serum (Amimed Cat #2-01F30-G, Lot #LB11566P). HCT 116 cells were cultured in growth medium composed of McCoys 5A (Amimed catalog #1-18F01-1), 2 mM L-Glutamine (Amimed Cat #5-10K50), 1× Penicillin-Streptomycin (Amimed Cat #4-01F00-H) and 10% fetal calf serum (Amimed Cat #2-01F30-G, Lot #LB11566P). All cells were maintained at 37° C. in a humidified 5% CO₂ incubator.

Following filtration through a Steriflip-NY 20 μm filter (Millipore Cat #SCNY00020), trypsinized cells were seeded in 100 microliters growth medium at 2′000 (SW48) or 1′500 (SNU-407, DLD1-WRN-KO, HCT 116) cells/well into white, clear-bottom 96-well plates (Costar Cat #3903). Three replicate plates were prepared for each compound treatment condition. In addition, one plate (termed “day 0”) was prepared to quantify the number of viable cells at the time of compound addition. Following overnight incubation at 37° C. in a humidified 5% CO2 atmosphere, eight 3-fold serial dilutions of a given compound stock (obtained at a concentration of 10 mM in DMSO and stored at 4° C.) were dispensed directly into each of the triplicate assay plates using a HP 300D non-contact Digital Dispenser (TECAN). The final concentration of DMSO was normalized to 0.1% in all wells. 96 hours after compound addition, cellular ATP levels as a surrogate for cell viability was assessed following addition of 50 microliters CellTiterGlo (Promega Cat #G7573) reagent and luminescence quantification on a MPLEX multi-mode plate-reader (TECAN) following a 10 minute incubation at room temperature. The number of viable cells in the “day 0” plate were quantified identically on the day of compound addition.

For data analysis, the assay background signal that was determined in wells containing medium, but no cells, was subtracted from all other data points prior to further calculations. The extent of growth inhibition and potential cell kill was assessed by comparing the ATP levels (measured using CellTiterGlo, Promega) in compound-treated cells with those present at the time of compound addition. To this end, the following conditional concept was programmatically applied in HELIOS, an in-house software applying a multi-step decision tree to arrive at optimal concentration response curve fits (Gubler et al, SLAS DOI: 10.1177/2472555217752140) to calculate % growth (% G) for each compound-treated well: % G=(T−V0)/V0))*100 when T<V0, and % G=(T−V0)/(V−V0)))*100 when T>V0, where V0 is the viability level at time of compound addition, while V and T represent vehicle-control and compound-treated viability levels, respectively, at the end of the compound incubation. 100%, 0% and −100% signify absence of growth inhibition, growth stasis, and complete cell kill, respectively. Compound concentrations leading to half-maximal growth inhibition (G150) and residual cell viability at the highest tested compound concentration (Data (cmax), expressed in percent) were routinely calculated. Data analysis can also be carried out using commercially available software designed to derive IC50 values using 4-parameter fits (e.g. GraphPad Prism, XL fit). The reported GI₅₀ values are the geometrical means of at least 2 independent replicates.

### Efficacy of WRN Inhibitors Against Subcutaneous SW48 Colorectal Xenografts

Experiments were performed in female Crl:NU(NCr)-Foxn1^{nu}-homozygous nude mice (Charles River). Animals were housed under Optimized Hygienic Conditions in Allentown XJ cages (IVC, max. 6 mice per cage) with food and water at libitum and a 12 h:12 h light:dark cycle. Animals were allowed to acclimatize for at least 1 week before being enrolled in the experimental design. The study described here was performed according to license 1975 approved by the Basel Cantonal Veterinary Office.

Tumors were established by subcutaneous inoculation of human colorectal cancer SW48 cells (5×10⁶ cells/animal in 100 μL in HBSS). SW48 human colorectal cancer cells were obtained from ATCC. The cells were cultured in RPMI-1640 medium (BioConcept Ltd., #1-41F01-1) supplemented with 10% FCS (Bio Concept #2-01F30-1), 2 mM L-glutamine (BioConcept Ltd., #5-10K50-H), 1 mM sodium pyruvate (Bio Concept #5-60F00-H) and 10 mM HEPES (Bio Concept #5-31F00-H) at 37° C. in an atmosphere of 5% CO₂ in air. To establish SW48 xenografts cells were harvested and re-suspended in HBSS (Sigma, #H6648) before injecting subcutaneously 100 μL containing 5 million cells in the right flank of animals which were anaesthetized with isoflurane. Tumor size, in mm³, was calculated following the formula: (L×W2×π/6); where W=width and L=length of the tumor. As a measure of efficacy the % T/C value is calculated at the end of the experiment according to:

$$( Δtumor {volume}^{treated} / Δtumor {volume}^{control} ) * 100$$

Tumor regression was calculated according to:

$$- ( Δtumor {volume}^{treated} / tumor {volume}^{treated at start} ) * 100$$

Where Δtumor volumes represent the mean tumor volume on the evaluation day minus the mean tumor volume at the start of the experiment.

Amorphous sodium salt of test compound (corrected by salt factor) was dissolved in an aqueous 20% w/v solution of 2-hydroxypropyl-beta-cyclodextrin (HPBCD). A range of concentrations was prepared by serial dilution starting from the solution at highest concentration for the highest dose in the study.

Treatment was initiated about two weeks post tumor cell inoculation, when the xenografts had reached a mean volume of approximately 200 mm³. The tumor bearing animals were randomized based on tumor volumes into experimental groups with 6 animals per group and treatment was initiated. Compounds were dosed orally (p.o.) once per day (qd) at 1, 3, 5 or 10 mg/kg (Example 18A) and at 0.1, 0.3, 1, 3 mg/kg (Example 21A). In addition, a control group treated once per day with vehicle (20% 2-hydroxypropyl-β-cyclodextrin) was included in each study. Tumor volumes and body weights were measured two times per week until the study termination.

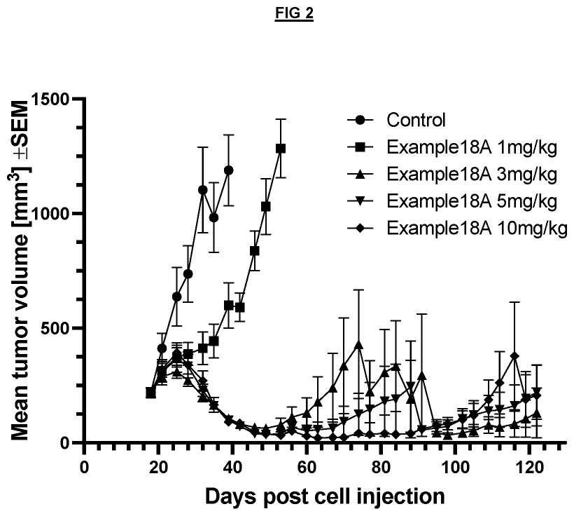

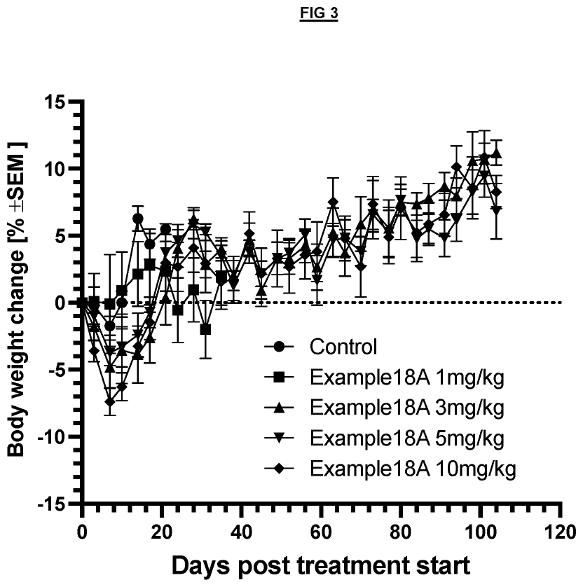

In the study assessing an efficacy of Example 18A the tumors in the vehicle treated group grew approximately linearly from about 200 to 1.100 mm³, dropping after thirty two days post injection due to termination of animals with large tumor mass (FIG. 1). Throughout the treatment duration the vehicle treated animals gained up to 9% of body weight at the treatment start (FIG. 2). After an initial body weight loss, not exceeding 14% among individual animals during first ten days of treatment, animals started gaining weight up to 18% of the body weight at start (FIG. 2). Dose dependent anti-tumor effect was observed (FIG. 1). At the 1 mg/kg dose level slight tumor growth delay was observed with % T/C reaching 22% on day 32 of the study (fourteenth day of treatment). Dosing at 3, 5 and 10 mg/kg resulted in a pronounced anti-tumor activity with maximal regression of 71% on day 49 of the study (thirty first day of treatment), 83% on day 49 (thirty first day of treatment) and 91% on day 63 (forty fifth day of treatment), respectively. Relapse was observed for all treatment groups, where the time of the relapse occurrence was dose dependent (FIG. 1). Abrupt decreases of the mean tumor volumes observed for groups treated at 3 and 5 mg/kg post ninetieth day of the study are a result of termination of animals with large tumor mass (FIG. 1).

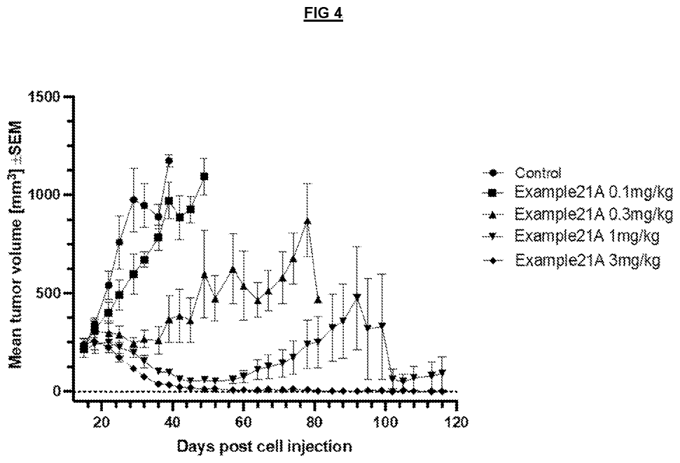

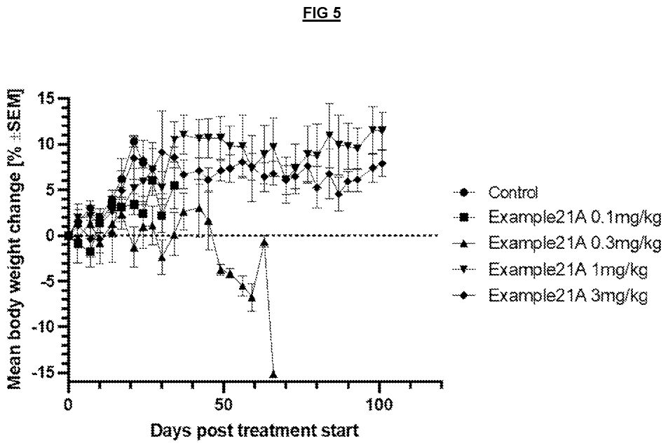

In the study assessing an efficacy of Example 21A the tumors in the vehicle treated group grew approximately linearly from about 200 to 1.000 mm³, dropping after twenty nine days post injection due to termination of animals with large tumor mass (FIG. 3). Throughout the treatment duration the vehicle treated animals gained up to 11% of body weight at the treatment start (FIG. 4). Example 21A was well tolerated throughout the study, with just minimal body weight loses among individual animals not exceeding 15% on individual occasions (FIG. 4). Body weight loss observed in the study is not dose-dependent and might have been non-treatment related. Dose dependent anti-tumor effect was observed (FIG. 3). At the 0.1 mg/kg dose level tumor growth delay was observed with % T/C reaching 50% from day 25 throughout 29^{th} day of the study (tenth to fourteenth day of treatment). At 0.3 mg/kg an initial stasis (% T/C reached 0% on day 29 of the study, on fourteenth day of treatment) was followed by the tumor regrowth post thirty sixth day from inoculation. Dosing at 1 and 3 mg/kg resulted in a pronounced anti-tumor activity with maximal regression of 75% on day 52 of the study (thirty seventh day of treatment) and 99% on day 85 (seventieth day of the treatment), respectively. While on treatment, no relapse was observed in three animals treated at 1 mg/kg and in entire group treated at 3 mg/kg with Example 21A. Abrupt decreases of the mean tumor volumes observed for groups treated at 0.3 and 1 mg/kg post eighty first and hundred second day of the study, respectively, are a result of termination of animals with large tumor mass (FIG. 1).

The following table shows the IC₅₀ data in the WRN ATPase assay and the GI₅₀ data for the proliferation assays using SW48 and DLD1-WRN-KO cell lines for compounds of the invention.

For example, Example 1A is a WRN ATPase inhibitor with a biochemical IC₅₀ of 0.02 μM and a proliferation GI₅₀ of 0.04 μM in SW48 and greater than 10 μM in the DLD1 WRN-KO cell lines.

| | WRN ATPase | Proliferation Assay | Proliferation Assay |
|---|---|---|---|
| | activity | SW48 | DLD1-WRN-KO |
| Example | IC₅₀ [μM] | GI₅₀ [μM] | GI₅₀ [μM] |
| 1A | 0.02 | 0.04 | >10 |
| 1B | 7 | 9 | >10 |
| 1C | 0.04 | 0.5 | >10 |
| 1D | 3 | 5 | >10 |
| 2A | 0.02 | 0.04 | >10 |
| 2B | 26 | >10 | >10 |
| 2C | 0.10 | 1 | >10 |
| 2D | 24 | >10 | >10 |
| 3A | 0.03 | 0.04 | >10 |
| 3B | 6 | >10 | >10 |
| 4A | 0.09 | 0.1 | >10 |
| 4B | 9 | 6 | >10 |
| 5A | 0.02 | 0.03 | >10 |
| 5B | 6 | 5 | >10 |
| 6A | 0.02 | 0.02 | >10 |
| 6B | 2 | 3 | >10 |
| 7 | 0.05 | 0.3 | >10 |
| 8 | 0.02 | 0.02 | >10 |
| 9 | 0.05 | 0.06 | >10 |
| 10 | 0.09 | 0.2 | >10 |
| 11 | 0.05 | 0.04 | >10 |
| 12 | 0.03 | 0.3 | >10 |
| 13 | 0.05 | 0.04 | >10 |
| 14A | 0.04 | 0.02 | >10 |
| 14B | 2 | 0.7 | >10 |
| 14C | 0.1 | 0.3 | >10 |
| 14D | 1 | 0.7 | >10 |
| 15A | 0.05 | 0.03 | >10 |
| 15B | 18 | 8 | >10 |
| 15C | 0.1 | 0.4 | >10 |
| 15D | 61 | >10 | >10 |
| 16 | 0.04 | 0.01 | >10 |
| 17 | 0.06 | 0.02 | >10 |
| 18A | 0.05 | 0.04 | >50 |
| 18B | 36 | >10 | >10 |
| 18C | >10 | >10 | >10 |
| 18D | 0.1 | 0.5 | >10 |
| 19A | 0.04 | 0.2 | >10 |
| 19B | 44 | | |
| 20A | 0.09 | 0.3 | >10 |
| 20B | >10 | >10 | >10 |
| 21A | 0.03 | 0.006 | >50 |
| 21C | 0.04 | 0.01 | >10 |
| 22 | 0.7 | 6 | >10 |
| 22B | >100 | | |
| 23A | 0.05 | 0.2 | >10 |
| 23B | 69 | >10 | >10 |
| 24 | 2 | 4 | >10 |
| 25 | | 0.01 | >10 |

Data are geometric means of at least 2 duplicate determinations except for the biochemical data of examples 10D and 22B.

In another aspect, the invention provides a compound of formula (I), which can be used as a research chemical, such as a tool compound or a chemical probe. In particular said use is in experiments relating to WRN inhibition or MSI high cancers.

### Preparation of Compounds

Compounds of the present invention can be prepared as described in the following Examples. The Examples are intended to illustrate the invention and are not to be construed as being limitations thereof.

### Instrumentation

### X-Ray Powder Diffraction Instrument and Method, FIG. 1:

| | |
|---|---|
| Instrument Model | Bruker D8 Discover new |
| Detector | VANTEC-500 |
| Radiation (Wavelength) | Cu (1.5418 Å) |
| X-ray generator power | 40 kV, 40 mA |
| Step size, resolution | Time/step 60 s, steps 2 |
| Scan range | 9° to 36° (2Theta value) |
| Scan time | 120 s |
| Temperature | Room temperature |
| Source slit | Optics-Goebel mirror-Cu, Microslit-10 |
| | height 1 mm, UBC-Collimator-G-10 |
| | width 1 mm |

### X-Ray Powder Diffraction Instrument and Method, FIG. 6:

| | |
|---|---|
| Instrument | Bruker D8 Advance |
| Detector | LynxEye (1D mode), open angle: 2.948° |
| Radiation (Wavelength) | CuKα (0.15418 nm) |
| Monochromator | Ni-filter |
| X-ray generator power | 40 kV, 40 mA |
| Step size | 0.0164° or 0.0410° (2Theta) |
| Time per step | 0.3 s |
| Scan range | 2°-40° 2theta |
| Scan time | 768 s or 279 s |
| Slit | Primary fixed illuminated sample size: 10 mm, |
| | secondary: open angle: 2.2°, axial soller: 2.5° |

The following alternative instrument method was used on a sample of compound of Example 21A with higher crystallinity:

| | |
|---|---|
| Instrument | Bruker D8 Advance |
| Detector | LYNXEYE_XE_T(1D mode) |
| Radiation | CuKα (0.15418 nm) |
| (Wavelength) | |
| Monochromator | Ni-filter |
| X-ray generator power | 40 kV, 40 mA |
| Step size | 0.016° (2Theta) |
| Time per step | 0.15 s |
| Scan range | 2° to 40° (2-theta value) |
| Scan time | About 383 seconds |
| Slits | Primary: fixed illuminated sample size 5 mm, |
| | axial soller 2.5° |
| | Secondary: open angle 2.38°, axial soller 2.5° |

This alternative method gave the following peaks:

| Angle | d | Rel. |
|---|---|---|
| 5.78 | 15.28 | 67.6% |
| 6.66 | 13.26 | 14.8% |
| 10.54 | 8.39 | 24.4% |
| 11.55 | 7.66 | 21.6% |
| 11.99 | 7.38 | 22.4% |
| 12.33 | 7.17 | 59.6% |
| 12.90 | 6.86 | 38.2% |
| 13.02 | 6.80 | 51.5% |
| 13.83 | 6.40 | 13.5% |
| 14.80 | 5.98 | 48.3% |
| 15.27 | 5.80 | 48.3% |
| 15.65 | 5.66 | 62.1% |
| 16.00 | 5.54 | 19.7% |
| 16.58 | 5.34 | 16.3% |
| 17.33 | 5.11 | 64.5% |
| 18.50 | 4.79 | 24.7% |
| 19.18 | 4.62 | 71.0% |
| 20.03 | 4.43 | 83.3% |
| 21.16 | 4.19 | 25.2% |
| 21.47 | 4.14 | 82.2% |
| 21.85 | 4.06 | 39.0% |
| 22.36 | 3.97 | 24.7% |
| 22.92 | 3.88 | 63.2% |
| 23.17 | 3.84 | 88.6% |
| 23.90 | 3.72 | 100.0% |

### UPLC-MS Methods:

Using Waters Acquity UPLC with Waters SQ detector, unless stated otherwise.

### UPLC-MS 1:

Column CORTECS™ C18+ 2.7 μm,
Column Dimension 2.1×50 mm
Column Temperature 80° C.
Eluents A: water+4.76% isopropanol+0.05% FA+3.75 mM AA
   B: isopropanol+0.05% FA
Flow Rate 1.0 mL/min
Gradient 1 to 50% B in 1.4 min; 50 to 98% B in 0.3 min

### UPLC-MS 2:

Column ACQUITY UPLC® BEH C18 1.7 μm
Column Dimension 2.1×100 mm
Column Temperature 80° C.
Eluents A: water+4.76% isopropanol+0.05% FA+3.75 mM AA
   B: isopropanol+0.05% FA
Flow Rate 0.4 mL/min
Gradient 1 to 60% B in 8.4 min; 60 to 98% B in 1.0 min

### UPLC-MS 3:

Column ACQUITY UPLC® BEH C18 1.7 μm
Column Dimension 2.1×50 mm
Column Temperature 80° C.
Eluents A: water+0.05% FA+3.75 mM AA
   B: isopropanol+0.05% FA
Flow Rate 0.6/0.7 mL/min
Gradient 5 to 98% B in 1.7 min

### UPLC-MS 4:

Column ACQUITY UPLC® BEH C18 1.7 μm
Column Dimension 2.1×100 mm
Column Temperature 80° C.
Eluents A: water+0.05% FA+3.75 mM AA
   B: isopropanol+0.05% FA
Flow Rate 0.4 mL/min
Gradient 5 to 60% B in 8.4 min; 60 to 98% B in 1.0 min

### UPLC-MS 5:

Column Acquity UPLC® HSS T3 1.8 μm
Column Dimension 2.1×50 mm
Column Temperature 50° C.
Eluents A: water+0.05% FA+3.75 mM AA
   B: acetonitrile+0.04% FA
Flow Rate 1.0 mL/min
Gradient 5 to 98% B in 1.4 min

### UPLC-MS 6:

Column Ascentis® Express C18 2.7 μm
Column Dimension 2.1×50 mm
Column Temperature 80° C.
Eluents A: water+4.76% isopropanol+0.05% FA+3.75 mM AA
   B: isopropanol+0.05% FA
Flow Rate 1.0 mL/min
Gradient 1 to 50% B in 1.4 min; 50-98% B in 0.3 min

### UPLC-MS 7:

Column XBridge® BEH™ C18 2.5 μm
Column Dimension 2.1×50 mm
Column Temperature 80° C.
Eluents A: water+5 mM NH₄OH
   B: acetonitrile+5 mM NH₄OH
Flow Rate 1.0 mL/min
Gradient 2 to 98% B in 1.4 min

### UPLC-MS 8:

Column ACQUITY UPLC® BEH C18 1.7 μm
Column Dimension 2.1×50 mm
Column Temperature 80° C.
Eluents A: water+4.76% isopropanol+0.05% FA+3.75 mM AA
   B: isopropanol+0.05% FA
Flow Rate 0.6 mL/min
Gradient 1 to 98% B in 1.7 min

### UPLC-MS 9:

Column CORTECS™ C18+ 2.7 μm,
Column Dimension 2.1×50 mm
Column Temperature 80° C.
Eluents A: water+0.05% FA+3.75 mM AA
   B: acetonitril+0.04% FA
Flow Rate 1.0 mL/min
Gradient 5 to 98% B in 1.4 min

### UPLC-MS 10:

Column CORTECS™ C18+ 2.7 μm,
Column Dimension 2.1×50 mm
Column Temperature 80° C.
Eluents A: water+0.05% FA+3.75 mM AA
   B: isopropanol+0.05% FA
Flow Rate 1.0 mL/min
Gradient concave from 1 to 98% B in 1.4 min

### HPLC Methods:

### HPLC 1:

Instrument Agilent 1260
Column Agilent Poroshell 120 EC-C18, 2.7 μm
Column Dimension 4.6×50 mm
Column Temperature 40° C.
Eluents A: water+0.1% TFA
   B: acetonitrile+0.1% TFA
Flow Rate 1.2 mL/min
Gradient 5% B to 95% B in 5 min, hold 2 min

### HPLC 2:

Instrument Agilent 1260 HPLC
Column InertSustain C18, 5 μm
Column Dimension 4.6×150 mm
Column Temperature 30° C.
Eluents A: water+5 mmol (NH₄)₂CO₃
   B: acetonitrile
Flow Rate 1.0 mL/min
Gradient 10% B to 90% B in 8 min, hold 2 min

### Chiral HPLC Methods:

### C-HPLC 1:

Instrument: analytical SFC-MS Waters UPC²
Injection: 5 μL
Mobile phase: A: 45% MeOH+0.1% NH₃, B: 55% scCO₂
Flow rate: 3 mL/min
Column: Chiralpak AD (4.6 mm×100 mm 5 μm)
Detection UV: DAD
Gradient: isocratic A: 45%, B: 55%
Oven Temperature: 40° C.
BPR: 1800 psi

### C-HPLC 2:

Instrument: analytical SFC-MS Waters UPC²
Injection: 5 μL
Mobile phase: A: 45% IPA+0.1% NH₃, B: 55% scCO₂
Flow rate: 3 mL/min
Column: Chiralpak IB (4.6 mm×100 mm 5 μm)
Detection UV: DAD
Gradient: isocratic A: 45%, B: 55%
Oven Temperature: 40° C.
BPR: 1800 psi

### C-HPLC 3:

Instrument: analytical SFC-MS Waters UPC²
Injection: 5 μL
Mobile phase: A: 45% MeOH+0.1% NH₃, B: 55% scCO₂
Flow rate: 3 mL/min
Column: Chiralpak IB-N (4.6 mm×100 mm 5 μm)
Detection UV: DAD
Gradient: isocratic A: 45%, B: 55%
Oven Temperature: 40° C.
BPR: 1800 psi

### C-HPLC 4:

Instrument: analytical SFC-MS Waters UPC²
Injection: 5 μL
Mobile phase: A: 40% MeOH+0.1% NH₃, B: 60% scCO₂
Flow rate: 3 mL/min
Column: Chiralpak IB-N (4.6 mm×100 mm 5 μm)
Detection UV: DAD
Gradient: isocratic A: 40%, B: 60%
Oven Temperature: 40° C.
BPR: 1800 psi

### C-HPLC 5:

Instrument: analytical SFC-MS Waters UPC²
Injection: 5 μL
Mobile phase: A: 55% MeOH+0.05% NH₃, B: 45% scCO₂
Flow rate: 3 mL/min
Column: Chiralpak IB (4.6 mm×100 mm 5 μm)
Detection UV: DAD
Gradient: isocratic A: 55%, B: 45%
Oven Temperature: 40° C.
BPR: 1800 psi

### C-HPLC 6:

Instrument: Ultimate3000
Injection: 4 μL
Mobile phase: A: 40% heptane with DEA, B: 60% EtOH with DEA;
Flow rate: 0.420 mL/min
Column: Chiralpak IG-3 (3.0 mm×100 mm 3 μm)
Detection UV: 240 nm
Gradient: isocratic A: 40%, B: 60%
Oven Temperature: 25° C.
BPR: 1800 psi

### C-HPLC 7:

Instrument: analytical SFC-MS Waters UPC²
Injection: 5 μL
Mobile phase: A: 40% MeOH+0.1% NH₃, B: 60% scCO₂
Flow rate: 3 mL/min
Column: Chiralpak IC (4.6 mm×100 mm 5 μm)
Detection UV: DAD
Gradient: isocratic A: 40%, B: 60%
Oven Temperature: 40° C.
BPR: 1800 psi

### C-HPLC 8:

Instrument: analytical SFC-MS Waters UPC²
Injection: 5 μL
Mobile phase: A: 45% MeOH+0.1% NH₃, B: 55% scCO₂
Flow rate: 3 mL/min
Column: Chiralpak IB-N (4.6 mm×100 mm 5 μm)
Detection UV: DAD
Gradient: isocratic A: 45%, B: 55%
Oven Temperature: 40° C.
BPR: 1800 psi

### C-HPLC 9:

Instrument: analytical SFC-MS Waters UPC²
Injection: 5 μL
Mobile phase: A: 25% MeOH+0.1% NH₃, B: 75% scCO₂
Flow rate: 3 mL/min
Column: Chiralpak IB (4.6 mm×100 mm 5 μm)
Detection UV: DAD
Gradient: isocratic A: 25%, B: 75%
Oven Temperature: 40° C.
BPR: 1800 psi

### C-HPLC 10:

Instrument: analytical SFC-MS Waters UPC²
Injection: 5 μL
Mobile phase: A: 40% MeOH+0.1% NH₃, B: 60% scCO₂
Flow rate: 3 mL/min
Column: Chiralpak IB (4.6 mm×100 mm 5 μm)
Detection UV: DAD
Gradient: isocratic A: 40%, B: 60%
Oven Temperature: 40° C.
BPR: 1800 psi

### C-HPLC 11:

Instrument: analytical SFC-MS Waters UPC²
Injection: 5 μL
Mobile phase: A: 35% MeOH+0.025% NH₃, B: 65% scCO₂
Flow rate: 3 mL/min
Column: Chiralpak IB-N (4.6 mm×100 mm 5 μm)
Detection UV: DAD
Gradient: isocratic A: 35%, B: 65%
Oven Temperature: 40° C.
BPR: 1800 psi

### C-HPLC 12:

Instrument: analytical SFC-MS Waters UPC²
Injection: 5 μL
Mobile phase: A: 20% MeOH+0.05% NH₃, B: 80% scCO₂
Flow rate: 3 mL/min
Column: Chiralpak AD (4.6 mm×100 mm 5 μm)
Detection UV: DAD
Gradient: isocratic A: 20%, B: 80%
Oven Temperature: 40° C.
BPR: 1800 psi

### C-HPLC 13:

Instrument: analytical SFC-MS Waters UPC²
Injection: 5 μL
Mobile phase: A: 50% MeOH+0.1% NH₃, B: 50% scCO₂
Flow rate: 3 mL/min
Column: Chiralpak IB-N (4.6 mm×100 mm 5 μm)
Detection UV: DAD
Gradient: isocratic A: 50%, B: 50%
Oven Temperature: 40° C.
BPR: 1800 psi

### C-HPLC 14:

Instrument: analytical SFC-MS Waters UPC²
Injection: 5 μL
Mobile phase: A: 10% MeOH+0.05% NH₃, B: 90% scCO₂
Flow rate: 3 mL/min
Column: Chiralpak IG (4.6 mm×100 mm 5 μm)
Detection UV: DAD
Gradient: isocratic A: 10%, B: 90%
Oven Temperature: 40° C.
BPR: 1800 psi

### Preparative Methods:

### Column Chromatography:

Column chromatography was run on silica gel using prepacked columns, as detailed below, or using glass columns following standard flash chromatography methodology, unless otherwise stated.
System 1 Teledyne ISCO, CombiFlash Rf, CombiFlash Rf+
System 2 Biotage Isolera
Column pre-packed RediSep Rf cartridges, or SNAP cartridges
Sample adsorption onto Isolute, or on silica gel, or applied as solutions

### Supercritical Fluid Chromatography (SFC):

Purifications were achieved on a Waters Preparative SFC-100-MS system with ABSYS update, with a Waters 2998 Photodiode Array Detector and a Waters MS Single Quadrupole Detector.

### SFC 1:

Instrument: WATERS SFC 100 with ABSYS update
Mobile phase: A: CO₂, B: MeOH
Flow rate: 150 mL/min MeOH+30 mL/min CO₂, constant flow of 180 mL/min
Column: 250×30 Reprospher PEI 100A 5 um
Temperature: 50° C.
Back pressure: 100 bar
Detection UV: 210-400 nm
Gradient: 23% B to 31% B in 7 min

### Reversed Phase HPLC:

### RP-HPLC Basic 1:

System Gilson
Column Waters X-Bridge Prep C18 OBD (100 mm×30 mm), 5 μm
Eluents A: water+7.3 mM NH₄OH, B: acetonitrile
Flow rate 40 mL/min

### RP-HPLC Acidic 1:

System Gilson
Column Waters SunFire Prep C18 OBD (100 mm×30 mm), 5 μm
Eluents A: water+0.1% TFA, B: acetonitrile
Flow rate 40 mL/min

### RP-HPLC Acidic 2:

System Teledyne/Isco AccqPrep HP150 prep
Column Xbridge C18 (50 mm×100 mm), 5 μm
Eluents A: water+0.1% TFA, B: acetonitrile
Flow rate 100 mL/min

### RP-HPLC Acidic 3:

System Waters Autopurification System with Waters QDa MS Detector
Column Xbridge Prep C18 (30 mm×100 mm), 5 μm
Eluents A: water+0.1% TFA, B: acetonitrile
Flow rate 1 mL/min

### Preparation of Compounds

The following examples are intended to illustrate the invention and are not to be construed as being limitations thereon. Temperatures are given in degrees Celsius. If not mentioned otherwise, all evaporations are performed under reduced pressure, typically between about 15 mm Hg and 100 mm Hg (=20-133 mbar). Abbreviations used are those conventional in the art.

All starting materials, building blocks, reagents, acids, bases, dehydrating agents, solvents, and catalysts utilized to synthesize the compounds of the present invention are either commercially available or can be produced by organic synthesis methods known to one of ordinary skill in the art. Further, the compounds of the present invention can be produced by organic synthesis methods known to one of ordinary skill in the art as shown in the following examples.

The structures of all final products, intermediates and starting materials are confirmed by standard analytical spectroscopic characteristics, e.g., MS, IR, NMR. The absolute stereochemistry of representative examples of the preferred (most active) isomers has been determined by analyses of X-ray crystal structures of complexes in which the respective compounds are bound to WRN or by small molecule X-ray crystal structures of a precursor or the final compound.

Amines synthesized via acidic deprotection of the Boc-precursor were often obtained as HCl or TFA salt. The corresponding free base can be isolated by partitioning between DCM and aq sat NaHCO₃ as described for Intermediate C.

### General Conditions:

Mass spectra were acquired on LC-MS systems using electrospray, chemical and electron impact ionization methods with a range of instruments of the following configurations: Waters Acquity UPLC with Waters SQ detector, analytical SFC-MS Waters UPC² and Agilent 1260 HPLC. [M+H]⁺ refers to the protonated molecular ion of the chemical species. [M−H]⁻ refers to the deprotonated molecular ion of the chemical species.

NMR spectra were run with Bruker Ultrashield™400 (400 MHz) and Bruker Ultrashield™600 (600 MHz) spectrometers, all with and without tetramethylsilane as an internal standard.

Chemical shifts (d-values) are reported in ppm downfield from tetramethylsilane, spectra splitting pattern are designated as singlet (s), doublet (d), triplet (t), multiplet (m), unresolved or more overlapping signals (m), broad signal (br). Solvents are given in parentheses.
Phase separator: Biotage—Isolute phase separator—(Part number: 120-1906-D for 15 mL, Part number: 120-1908-F for 70 mL and Part number: 120-1909-J for 150 mL)
SiliaMetS®Thiol: SiliCYCLE thiol metal scavenger—(Part number: R51030B, Loading: 1.31 mmol/g Particle Size: 40-63 μm)
ISOLUTE® Si-TMT: Biotage thiol metal scavenger—(Part number: 9538-0100, Loading: 0.49 mmol/g).

### Sodium Salt Formation:

The compound was suspended in tert-butanol. NaOH 0.1M (1 eq) was added. The mixture was stirred/sonicated at RT. If the suspension turned into a clear solution it was lyophilized. If the suspension was still turbid, water was added and the resulting solution was lyophilized. If no change happened, NaOH 0.1M up to 2 eq in total was added until a clear solution was observed, which was then lyophilized. If the NMR of the resulting solid still contained tert-butanol, the solid was dissolved in a small amount of water and lyophilized again. The final sodium salts were obtained as colourless powders. The amorphous state was confirmed by XRPD.

### Abbreviations

| Abbreviation | Description |
|---|---|
| Φ | Diameter |
| AA | ammonium acetate |
| ACN | Acetonitrile |
| AcOH | acetic acid |
| aq | Aqueous |
| BBr₃ | boron tribromide |
| BEMP | 2-(tert-butylimino)-N,N-diethyl-1,3-dimethyl- |
| | 1,3,2\|5-diazaphosphinan-2-amine |
| Boc | tert-butyloxycarbonyl |
| Boc₂O | di-tert-butyl dicarbonate |
| BPR | back pressure regulation |
| br | broad signal |
| brine | saturated aqueous sodium chloride |
| ° C. | degrees Celsius |
| CaCl₂ | calcium chloride |
| CDI | di(1H-imidazol-1-yl)methanone |
| CO | carbon monoxide |
| CO₂ | carbon dioxide |
| DAD | diode array detector |
| DCM | Dichloromethane |
| DIBAL-H | diisobutylaluminium hydride |
| DIPEA | N,N-diisopropylethylamine |
| DMAP | 4-dimethylaminopyridine |
| DMF | N,N-dimethylformamide |
| DMSO | dimethyl sulfoxide |
| DMSO-d₆ | hexadeuterodimethyl sulfoxide |
| ee | enantiomeric excess |
| eq | Equivalent |
| Et₃N | Triethylamine |
| Et₂O | diethyl ether |
| EtOAc | ethyl acetate |
| EtOH | Ethanol |
| FA | formic acid |
| H₂O | Water |
| HATU | 1-[Bis(dimethylamino)methylene]-1H-1,2,3-triazo- |
| | lo[4,5-b]pyridinium 3-oxide hexafluorophosphate |
| HBr | hydrobromic acid |
| HCl | hydrochloric acid |
| HCOOH | formic acid |
| Hg | Mercury |
| HNO₃ | nitric acid |
| HPLC | high-performance liquid chromatography |
| H₃PO₄ | phosphoric acid |
| HV | high vacuum |
| Hz, MHz, GHz | hertz, megahertz, gigahertz |
| IPA | isopropyl alcohol |
| IPAc | isopropyl acetate |
| J | coupling constant |
| KBr | potassium bromide |
| K₂CO₃ | potassium carbonate |
| KF | potassium fluoride |
| kg/g/mg | kilogram/gram/milligram |
| K₃PO₄ | potassium phosphate |
| l | Length |
| L/mL/μL | liter/milliliter/microliter |
| LC-MS | liquid chromatography-mass spectrometry |
| LDA | lithium diisopropylamide |
| LiHMDS | lithium bis(trimethylsilyl)amide |
| LiOH | lithium hydroxide |
| M/mM | molar/millimolar |
| mbar | Millibar |
| MeOH | Methanol |
| MeTHF | 2-methyloxolane/2-methyltetrahydrofuran |
| MgSO₄ | magnesium sulfate |
| min | Minutes |
| mm/nm/μm | millimeter/nanometer/micrometer |
| μM/mM | micromolar/millimolar |
| μmol/mmol/mol | micromol/millimol/mol |
| MOMCl | chloro(methoxy)methane |
| MS | mass spectrometry |
| MW, mw | Microwave |
| m/z | mass to charge ratio |
| N | Normality |
| N₂ | Nitrogen |
| NaCl | sodium chloride |
| NaCN | sodium cyanide |
| NaH | sodium hydride |
| NaHCO₃ | sodium bicarbonate |
| NaHMDS | sodium bis(trimethylsilyl) amide |
| NaHSO₄ | sodium bisulfate |
| NaOH | sodium hydroxide |
| Na₂SO₃ | sodium sulfite |
| Na₂S₂O₃ | sodium thiosulfate |
| NaOtBu | sodium 2-methylpropan-2-olate |
| Na₂SO₄ | sodium sulfate |
| NBS | N-bromosuccinimide |
| NH₃ | Ammonia |
| NH₄Cl | ammonium chloride |
| NH₄OH | ammonium hydroxide |
| NMP | 1-methylpyrrolidin-2-one |
| NMR | nuclear magnetic resonance |
| Pd—C | palladium on carbon |
| Pd(dppf)Cl₂•DCM | [1,1′-Bis(diphenylphosphino)ferrocene]palladium(II) |
| | dichloride, DCM adduct |
| pTSOH | para-toluenesulfonic acid |
| RM or rm | reaction mixture |
| RP | reversed phase |
| Rt | retention time (if not indicated, in minutes) |
| RT | room temperature |
| s, d, dd, t, m | singulet, doublet, doublet of doublets, triplet, |
| | multiplet |
| sat | Saturated |
| scCO₂ | supercritical CO₂ |
| SEMCl | 1 2-(trimethylsilyl)ethoxymethyl chloride |
| SFC | supercritical fluid chromatography |
| T₃P | propanephosphonic acid anhydride |
| TBAF | tetrabutylammonium fluoride |
| TBME | 2-methoxy-2-methylpropane |
| TEMPO | (2,2,6,6-tetramethylpiperidin-1-yl)oxyl |
| TFA | trifluoroacetic acid |
| THF | Tetrahydrofuran |
| TiCl₄ | titanium tetrachloride |
| Ti(OiPr)₄ | titanium isopropoxide |
| UPLC | ultra performance liquid chromatography |
| UV | ultra violet |
| wt % | percentage by weight |
| XPhos Pd G3 | (2-dicyclohexylphosphino-2′,4′,6′-triisopropyl-1,1′- |
| | biphenyl)[2-(2′-amino-1,1′-biphenyl)]palladium(II) |
| | methanesulfonate |
| XRPD | X-Ray powder diffraction |

### Preparation of Final Compounds

### Example 1A: (7R,9R)-6-(4-(4-chloro-3-hydroxypicolinoyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide and Example 1B: (7S,9S)-6-(4-(4-chloro-3-hydroxypicolinoyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide and Example 10: (7S,9R)-6-(4-(4-chloro-3-hydroxypicolinoyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide and Example 10D: (7R,9S)-6-(4-(4-chloro-3-hydroxypicolinoyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide

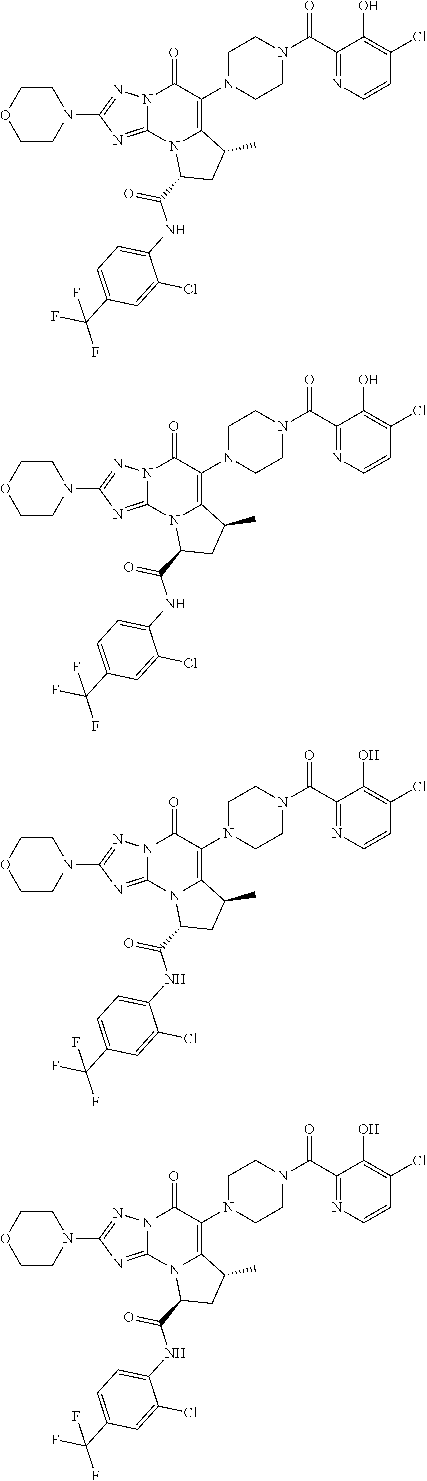 [SMILES: C[C@@H]1C[C@H](C(=O)Nc2ccc(C(F)(F)F)cc2Cl)n2c1c(N1CCN(C(=O)c3nccc(Cl)c3O)CC1)c(=O)n1nc(N3CCOCC3)nc21 ; C[C@H]1C[C@@H](C(=O)Nc2ccc(C(F)(F)F)cc2Cl)n2c1c(N1CCN(C(=O)c3nccc(Cl)c3O)CC1)c(=O)n1nc(N3CCOCC3)nc21 ; C[C@H]1C[C@H](C(=O)Nc2ccc(C(F)(F)F)cc2Cl)n2c1c(N1CCN(C(=O)c3nccc(Cl)c3O)CC1)c(=O)n1nc(N3CCOCC3)nc21 ; C[C@@H]1C[C@@H](C(=O)Nc2ccc(C(F)(F)F)cc2Cl)n2c1c(N1CCN(C(=O)c3nccc(Cl)c3O)CC1)c(=O)n1nc(N3CCOCC3)nc21]

N-(2-chloro-4-(trifluoromethyl)phenyl)-7-methyl-2-morpholino-5-oxo-6-(piperazin-1-yl)-5,7,8,9-tetrahydropyrrolo[1,2-c][1,2,4]triazolo[1,5-a]pyrimidine-9-carboxamide (Intermediate C) (707 mg, 1.22 mmol), 4-chloro-3-hydroxypicolinic acid (232 mg, 1.34 mmol) and HATU (486 mg, 1.28 mmol) were mixed DCM (15 mL) and cooled to 0° C. Then DIPEA (531 μL, 3.04 mmol) was added and the mixture was stirred at RT for 2 days. Water (10 mL), aq sat NaHCO₃ (10 mL) and DCM (10 mL) were added. The aqueous layer was washed with DCM (2×10 mL). The combined organic layers were dried through a phase separator and concentrated under reduced pressure. The crude product was purified by reverse phase preparative ISCO (RediSep Column: C18 50 g gold, eluent water+0.1% TFA:ACN 95:5 to 0:100 in 20 min). The product containing fractions were combined, basified with aq sat NaHCO₃, extracted twice with DCM, dried through a phase separator and concentrated under reduced pressure. The residue was further purified in 3 portions by reverse phase preparative HPLC (RP-HPLC acidic 1: 20 to 80% B in 20 min with a plateau at 45% for 3 min and at 55% for 3 min), (RP-HPLC acidic 1: 35 to 75% B in 19 min with a plateau at 45% for 2 min and at 55% for 3 min) and (RP-HPLC acidic 1: 35 to 75% B in 16 min with a plateau at 50% for 3 min). All fractions containing the first eluting peak, purity>90%, were combined, basified with aq sat NaHCO₃, extracted twice with DCM, dried through a phase separator and concentrated under reduced pressure. All fractions containing the second eluting peak, purity>90%, were combined, basified with aq sat NaHCO₃, extracted twice with DCM, dried through a phase separator and concentrated under reduced pressure. All other fractions containing the first eluting peak and/or the second eluting peak were combined, basified with aq sat NaHCO₃, extracted twice with DCM, dried through a phase separator, concentrated under reduced pressure and purified by reverse phase preparative HPLC (RP-HPLC basic 1: 20 to 80% B in 20 min). All fractions containing the first eluting peak, purity>90%, were combined, basified with aq sat NaHCO₃, extracted twice with DCM, dried through a phase separator, concentrated under reduced pressure and combined with the first batch. All fractions containing the second eluting peak, purity>90%, were combined, basified with aq sat NaHCO₃, extracted twice with DCM, dried through a phase separator, concentrated under reduced pressure and combined with the first batch.

The first eluting peak (racemate of 2 enantiomers—180 mg) was purified by preparative chiral SFC (instrument: Sepiatec prep SFC-100; column: LUX Amylose-1 (Chiralpak-AD), 250 mm×30 mm 5 μm; eluent: A: 45% MeOH+0.1% NH₃, B: 55% scCO₂; flow rate: 80.0 mL/min; detection: UV; injection volume: 1 mL; gradient: isocratic A: 45%, B: 55%; oven temperature: 40° C.; BPR: 105 bar).

### Example 1C: (7S,9R)-6-(4-(4-chloro-3-hydroxypicolinoyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide

The first eluting stereoisomer of chiral separation: 75.0 mg, 99% pure, yield: 8%

LC-MS: Rt=1.05 min; MS m/z [M+H]⁺ 736.5/738.5, m/z [M−H]⁻ 734.3/736.3; UPLC-MS 1

Chiral HPLC (C-HPLC 1): Rt=1.08 min, 99.5% ee

### Example 1D: (7R,9S)-6-(4-(4-chloro-3-hydroxypicolinoyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide

The second eluting stereoisomer of chiral separation: 80.0 mg, 99% pure, yield: 9%

LC-MS: Rt=1.07 min; MS m/z [M+H]⁺ 736.5/738.4, m/z [M−H]⁻ 734.2/736.2; UPLC-MS 1

Chiral HPLC (C-HPLC 1): Rt=1.84 min, 99.5% ee

The second eluting peak (racemate of 2 enantiomers—152 mg) was purified by preparative chiral SFC (instrument: Sepiatec prep SFC-100; column: LUX Amylose-1 (Chiralpak AD), 250 mm×30 mm 5 μm; eluent: A: 48% MeOH+0.1% NH₃, B: 52% scCO₂; flow rate: 85.0 mL/min; detection: UV; injection volume: 2.5 mL; gradient: isocratic A: 48%, B: 52%; oven temperature: 40° C.; BPR: 100 bar).

### Example 1B: (7S,9S)-6-(4-(4-chloro-3-hydroxypicolinoyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide

The first eluting stereoisomer of chiral separation: 65.0 mg, 99% pure, yield: 7%

The sodium salt was prepared analogous to the general procedure.

LC-MS: Rt=1.07 min; MS m/z [M+H]⁺ 736.5/738.6, m/z [M−H]⁻ 734.3/736.3; UPLC-MS 1

Chiral HPLC (C-HPLC 1): Rt=1.02 min, 99.5% ee

### Example 1A: (7R,9R)-6-(4-(4-chloro-3-hydroxypicolinoyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide

The second eluting stereoisomer of chiral separation: 72.0 mg, 95% pure, yield: 7.5%

The sodium salt was prepared analogous to the general procedure.

LC-MS: Rt=1.07 min; MS m/z [M+H]⁺ 736.4/738.5, m/z [M−H]⁻ 734.3/736.4; UPLC-MS 1

Chiral HPLC (C-HPLC 1): Rt=1.80 min, 99.5% ee

The stereochemistry of example 1A as (7R,9R) and 1C (7S,9R) was assigned based on their potency and an understanding of structure-activity relationships.

### Example 2A: (7R,9R)—N-(2-chloro-4-(trifluoromethyl)phenyl)-6-(4-(5-hydroxy-6-methylpyrimidine-4-carbonyl)piperazin-1-yl)-7-methyl-2-morpholino-5-oxo-5,7,8,9-tetrahydropyrrolo[1,2-c][1,2,4]triazolo[1,5-a]pyrimidine-9-carboxamide and Example 2B: (7S,9S)—N-(2-chloro-4-(trifluoromethyl)phenyl)-6-(4-(5-hydroxy-6-methylpyrimidine-4-carbonyl)piperazin-1-yl)-7-methyl-2-morpholino-5-oxo-5,7,8,9-tetrahydropyrrolo[1,2-c][1,2,4]triazolo[1,5-a]pyrimidine-9-carboxamide and Example 2C: (7S,9R)—N-(2-chloro-4-(trifluoromethyl)phenyl)-6-(4-(5-hydroxy-6-methylpyrimidine-4-carbonyl)piperazin-1-yl)-7-methyl-2-morpholino-5-oxo-5,7,8,9-tetrahydropyrrolo[1,2-c][1,2,4]triazolo[1,5-a]pyrimidine-9-carboxamide and Example 2D: (7R,9S)—N-(2-chloro-4-(trifluoromethyl)phenyl)-6-(4-(5-hydroxy-6-methylpyrimidine-4-carbonyl)piperazin-1-yl)-7-methyl-2-morpholino-5-oxo-5,7,8,9-tetrahydropyrrolo[1,2-c][1,2,4]triazolo[1,5-a]pyrimidine-9-carboxamide

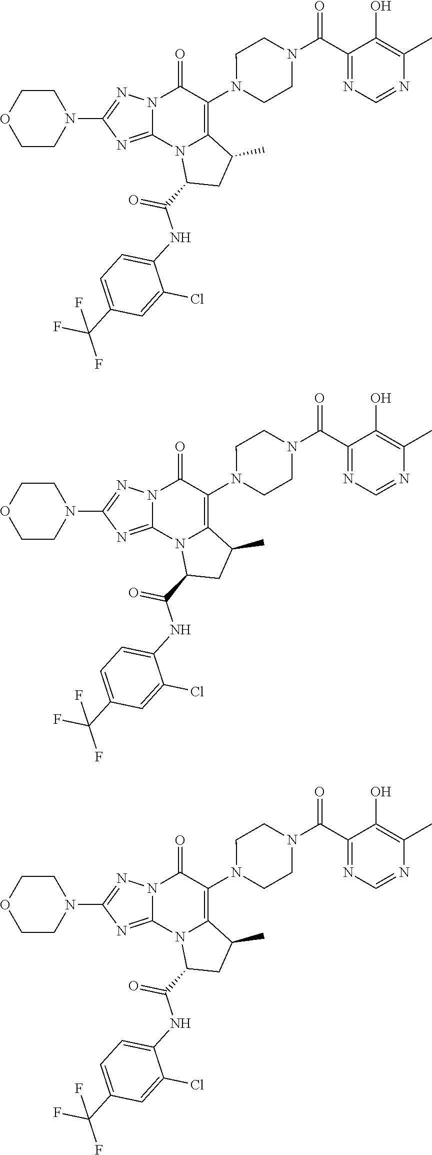 [SMILES: Cc1ncnc(C(=O)N2CCN(c3c4n(c5nc(N6CCOCC6)nn5c3=O)[C@@H](C(=O)Nc3ccc(C(F)(F)F)cc3Cl)C[C@H]4C)CC2)c1O ; Cc1ncnc(C(=O)N2CCN(c3c4n(c5nc(N6CCOCC6)nn5c3=O)[C@H](C(=O)Nc3ccc(C(F)(F)F)cc3Cl)C[C@@H]4C)CC2)c1O ; Cc1ncnc(C(=O)N2CCN(c3c4n(c5nc(N6CCOCC6)nn5c3=O)[C@@H](C(=O)Nc3ccc(C(F)(F)F)cc3Cl)C[C@@H]4C)CC2)c1O]

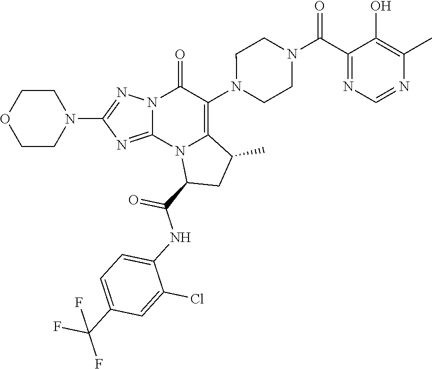 [SMILES: Cc1ncnc(C(=O)N2CCN(c3c4n(c5nc(N6CCOCC6)nn5c3=O)[C@H](C(=O)Nc3ccc(C(F)(F)F)cc3Cl)C[C@H]4C)CC2)c1O]

### Step 1: (7R,9S)-6-(4-(5-(benzyloxy)-6-methylpyrimidine-4-carbonyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide and (7S,9R)-6-(4-(5-(benzyloxy)-6-methylpyrimidine-4-carbonyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide and (7S,9S)-6-(4-(5-(benzyloxy)-6-methylpyrimidine-4-carbonyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide and (7R,9R)-6-(4-(5-(benzyloxy)-6-methylpyrimidine-4-carbonyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide

N-(2-chloro-4-(trifluoromethyl)phenyl)-7-methyl-2-morpholino-5-oxo-6-(piperazin-1-yl)-5,7,8,9-tetrahydropyrrolo[1,2-c][1,2,4]triazolo[1,5-a]pyrimidine-9-carboxamide (Intermediate C) (650 mg, 1.12 mmol), 5-(benzyloxy)-6-methylpyrimidine-4-carboxylic acid (Intermediate U) (301 mg, 1.23 mmol) and HATU (447 mg, 1.18 mmol) were mixed in DCM (20 mL) and cooled to 0° C. Then DIPEA (489 μL, 2.80 mmol) was added. The mixture was stirred at RT for 1.5 hours. Water (10 mL), aq sat NaHCO₃ (10 mL) and DCM (10 mL) were added. The aqueous layer was washed with DCM (2×10 mL). The combined organic layers were dried through a phase separator and concentrated under reduced pressure. The crude product was purified in 6 portions by reverse phase preparative HPLC (RP-HPLC acidic 1: 25 to 85% B in 20 min with a plateau at 65% for 1 min), (RP-HPLC acidic 1: 40 to 75% B in 20 min), (RP-HPLC acidic 1: 40 to 70% B in 18 min with a plateau at 60% for 1 min), (RP-HPLC acidic 1: 48 to 63% B in 17 min), (RP-HPLC acidic 1: 48 to 61% B in 17 min) and (RP-HPLC acidic 1: 45 to 60% B in 15 min with a plateau at 50% for 2 min). All fractions containing the first eluting peak were combined, basified with aq sat NaHCO₃, extracted twice with DCM, dried through a phase separator and concentrated under reduced pressure to give a racemic mixture of (7R,9S)-6-(4-(5-(benzyloxy)-6-methylpyrimidine-4-carbonyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide and (7S,9R)-6-(4-(5-(benzyloxy)-6-methylpyrimidine-4-carbonyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide.

LC-MS: Rt=1.14 min; MS m/z [M+H]⁺ 807.6/809.6, m/z [M−H]⁻ 805.5/807.4; UPLC-MS 1 All fractions containing the second eluting peak were combined, basified with aq sat NaHCO₃, extracted twice with DCM, dried through a phase separator and concentrated under reduced pressure to give a racemic mixture of (7S,9S)-6-(4-(5-(benzyloxy)-6-methylpyrimidine-4-carbonyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide and (7R,9R)-6-(4-(5-(benzyloxy)-6-methylpyrimidine-4-carbonyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide.

LC-MS: Rt=1.16 min; MS m/z [M+H]⁺ 807.5/809.5, m/z [M−H]⁻ 805.3/807.2; UPLC-MS 1

### Step 2a: (7R,9S)—N-(2-chloro-4-(trifluoromethyl)phenyl)-6-(4-(5-hydroxy-6-methylpyrimidine-4-carbonyl)piperazin-1-yl)-7-methyl-2-morpholino-5-oxo-5,7,8,9-tetrahydropyrrolo[1,2-c][1,2,4]triazolo[1,5-a]pyrimidine-9-carboxamide and (7S,9R)—N-(2-chloro-4-(trifluoromethyl)phenyl)-6-(4-(5-hydroxy-6-methylpyrimidine-4-carbonyl)piperazin-1-yl)-7-methyl-2-morpholino-5-oxo-5,7,8,9-tetrahydropyrrolo[1,2-c][1,2,4]triazolo[1,5-a]pyrimidine-9-carboxamide

The racemic mixture of (7R,9S)-6-(4-(5-(benzyloxy)-6-methylpyrimidine-4-carbonyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide and (7S,9R)-6-(4-(5-(benzyloxy)-6-methylpyrimidine-4-carbonyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide (the first eluting peak) (325 mg, 403 μmol) was dissolved in DCM (5 mL) and TFA (5.00 mL, 64.9 mmol) was added. The mixture was stirred at 60° C. overnight. The mixture was concentrated under reduced pressure. The crude product was purified in 2 portions by reverse phase preparative HPLC (RP-HPLC acidic 1: 20 to 80% B in 20 min with a plateau at 40% for 1 min and at 50% for 2 min) and (RP-HPLC acidic 1: 25 to 75% B in 20 min). The product containing fractions were combined, basified with aq sat NaHCO₃, extracted twice with DCM, dried through a phase separator and concentrated under reduced pressure.

The racemate (224 mg, 95% pure) was purified by preparative chiral SFC (instrument: Sepiatec prep SFC-100; column: Chiralpak IB-N 250 mm×30 mm 5 μm; eluent: A: 45% IPA+0.1% NH₃, B: 55% scCO₂; flow rate: 80.0 mL/min; detection: UV; injection volume: 1.1 mL; gradient: isocratic A: 45%, B: 52%; oven temperature: 40° C.; BPR: 110 bar).

### The First Eluting Stereoisomer: (7R,9S)—N-(2-chloro-4-(trifluoromethyl)phenyl)-6-(4-(5-hydroxy-6-methylpyrimidine-4-carbonyl)piperazin-1-yl)-7-methyl-2-morpholino-5-oxo-5,7,8,9-tetrahydropyrrolo[1,2-c][1,2,4]triazolo[1,5-a]pyrimidine-9-carboxamide

99.0 mg, 96% pure, yield: 33%

LC-MS: Rt=0.98 min; MS m/z [M+H]⁺ 717.6/719.6, m/z [M−H]⁻ 715.4/717.4; UPLC-MS 1

LC-MS: Rt=4.86 min; MS m/z [M+H]⁺ 717.5/719.6, m/z [M−H]⁻ 715.4/717.4; UPLC-MS 2

Chiral HPLC (C-HPLC 2): Rt=1.51 min, 99.5% ee

### The Second Eluting Stereoisomer: (7S,9R)—N-(2-chloro-4-(trifluoromethyl)phenyl)-6-(4-(5-hydroxy-6-methylpyrimidine-4-carbonyl)piperazin-1-yl)-7-methyl-2-morpholino-5-oxo-5,7,8,9-tetrahydropyrrolo[1,2-c][1,2,4]triazolo[1,5-a]pyrimidine-9-carboxamide

90.0 mg, 100% pure, yield: 31%

LC-MS: Rt=0.99 min; MS m/z [M+H]⁺ 717.6/719.7, m/z [M−H]⁻ 715.2/717.3; UPLC-MS 1

LC-MS: Rt=4.90 min; MS m/z [M+H]⁺ 717.5/719.5, m/z [M−H]⁻ 715.5/717.4; UPLC-MS 2

Chiral HPLC (C-HPLC 2): Rt=2.07 min, 97.2% ee

### Step 2b: (7R,9R)—N-(2-chloro-4-(trifluoromethyl)phenyl)-6-(4-(5-hydroxy-6-methylpyrimidine-4-carbonyl)piperazin-1-yl)-7-methyl-2-morpholino-5-oxo-5,7,8,9-tetrahydropyrrolo[1,2-c][1,2,4]triazolo[1,5-a]pyrimidine-9-carboxamide and (7S,9S)—N-(2-chloro-4-(trifluoromethyl)phenyl)-6-(4-(5-hydroxy-6-methylpyrimidine-4-carbonyl)piperazin-1-yl)-7-methyl-2-morpholino-5-oxo-5,7,8,9-tetrahydropyrrolo[1,2-c][1,2,4]triazolo[1,5-a]pyrimidine-9-carboxamide

The racemic mixture of (7S,9S)-6-(4-(5-(benzyloxy)-6-methylpyrimidine-4-carbonyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide and (7R,9R)-6-(4-(5-(benzyloxy)-6-methylpyrimidine-4-carbonyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide (the second eluting peak) (359 mg, 414 μmol) was dissolved in DCM (5 mL) and TFA (5.00 mL, 64.9 mmol) was added. The mixture was stirred at 60° C. overnight. The mixture was concentrated under reduced pressure. The crude product was purified in 2 portions by reverse phase preparative HPLC (RP-HPLC basic 1: 25 to 75% B in 20 min) and (RP-HPLC basic 1: 20 to 65% B in 20 min). The product containing fractions were combined, extracted twice with DCM, dried through a phase separator and concentrated under reduced pressure.

The racemate (236 mg) was purified by preparative chiral HPLC (instrument: Waters Prep SFC100-MS; column: Chiralpak IB-N 250 mm×30 mm 5 μm; eluent: A: 45% MeOH+0.1% NH₃, B: 55% scCO₂; flow rate: 80.0 mL/min; detection: DAD; injection volume: 1 mL; gradient: isocratic A: 45%, B: 55%; oven temperature: 40° C.; BPR: 120 bar).

### First Eluting Stereoisomer: (7S,9S)—N-(2-chloro-4-(trifluoromethyl)phenyl)-6-(4-(5-hydroxy-6-methylpyrimidine-4-carbonyl)piperazin-1-yl)-7-methyl-2-morpholino-5-oxo-5,7,8,9-tetrahydropyrrolo[1,2-c][1,2,4]triazolo[1,5-a]pyrimidine-9-carboxamide

62.2 mg, 87% pure

The first eluting stereoisomer was purified by reverse phase preparative HPLC (RP-HPLC acidic 2: 5 to 100% B in 30 min). The product containing fractions were combined, basified with aq sat NaHCO₃, extracted twice with DCM, dried through a phase separator and concentrated under reduced pressure to give the title compound (47.8 mg, 99% pure, yield: 16%).

The sodium salt was prepared analogous to the general procedure.

LC-MS: Rt=1.01 min; MS m/z [M+H]⁺ 717.5/719.5, m/z [M−H]⁻ 715.3/717.4; UPLC-MS 1

Chiral HPLC (C-HPLC 3): Rt=1.00 min, 97.5% ee

### Second Eluting Stereoisomer: (7R,9R)—N-(2-chloro-4-(trifluoromethyl)phenyl)-6-(4-(5-hydroxy-6-methylpyrimidine-4-carbonyl)piperazin-1-yl)-7-methyl-2-morpholino-5-oxo-5,7,8,9-tetrahydropyrrolo[1,2-c][1,2,4]triazolo[1,5-a]pyrimidine-9-carboxamide

41.3 mg, 100% pure, yield: 14%

The sodium salt was prepared analogous to the general procedure.

LC-MS: Rt=1.00 min; MS m/z [M+H]⁺ 717.6/719.6, m/z [M−H]⁻ 715.5/717.4; UPLC-MS 1

Chiral HPLC (C-HPLC 3): Rt=3.21 min, 99.5% ee

The stereochemistry of example 2A as (7R,9R) and 2C (7S,9R) was assigned based on their potency and an understanding of structure-activity relationships.

### Example 3A: (7R,9R)—N-(2-chloro-4-(trifluoromethyl)phenyl)-6-(4-(4-fluoro-3-hydroxy-6-methylpicolinoyl)piperazin-1-yl)-7-methyl-2-morpholino-5-oxo-5,7,8,9-tetrahydropyrrolo[1,2-c][1,2,4]triazolo[1,5-a]pyrimidine-9-carboxamide and Example 3B: (7S,9S)—N-(2-chloro-4-(trifluoromethyl)phenyl)-6-(4-(4-fluoro-3-hydroxy-6-methylpicolinoyl)piperazin-1-yl)-7-methyl-2-morpholino-5-oxo-5,7,8,9-tetrahydropyrrolo[1,2-c][1,2,4]triazolo[1,5-a]pyrimidine-9-carboxamide

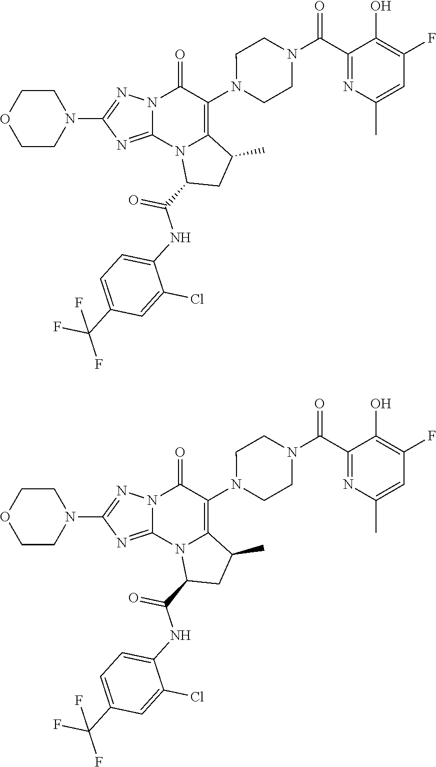 [SMILES: Cc1cc(F)c(O)c(C(=O)N2CCN(c3c4n(c5nc(N6CCOCC6)nn5c3=O)[C@@H](C(=O)Nc3ccc(C(F)(F)F)cc3Cl)C[C@H]4C)CC2)n1 ; Cc1cc(F)c(O)c(C(=O)N2CCN(c3c4n(c5nc(N6CCOCC6)nn5c3=O)[C@H](C(=O)Nc3ccc(C(F)(F)F)cc3Cl)C[C@@H]4C)CC2)n1]

### Step 1: (7S,9S)-6-(4-(3-(benzyloxy)-4-fluoro-6-methylpicolinoyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide and (7R,9R)-6-(4-(3-(benzyloxy)-4-fluoro-6-methylpicolinoyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide

The racemic mixture of (7R,9R)—N-(2-chloro-4-(trifluoromethyl)phenyl)-7-methyl-2-morpholino-5-oxo-6-(piperazin-1-yl)-5,7,8,9-tetrahydropyrrolo[1,2-c][1,2,4]triazolo[1,5-a]pyrimidine-9-carboxamide and (7S,9S)—N-(2-chloro-4-(trifluoromethyl)phenyl)-7-methyl-2-morpholino-5-oxo-6-(piperazin-1-yl)-5,7,8,9-tetrahydropyrrolo[1,2-c][1,2,4]triazolo[1,5-a]pyrimidine-9-carboxamide (Intermediate D) (483 mg, 695 μmol), 3-(benzyloxy)-4-fluoro-6-methylpicolinic acid (Intermediate V) (232 mg, 888 μmol) and HATU (277 mg, 730 μmol) were mixed in DCM (5 mL) and DIPEA (350 μL, 2.00 mmol) was added. The white suspension turned into a pale yellow solution and was stirred at RT for 1 hour. Water (10 mL), aq sat NaHCO₃ (10 mL) and DCM (10 mL) were added. The aqueous layer was washed with DCM (2×10 mL). The combined organic layers were dried through a phase separator and concentrated under reduced pressure. The crude product was purified by reverse phase preparative HPLC (RP-HPLC acidic 2: 35 to 65% B in 20 min). The product containing fractions were combined, basified with aq sat NaHCO₃, extracted twice with DCM, dried through a phase separator and concentrated under reduced pressure to give the title compound.

This racemate (434 mg) was purified by preparative chiral HPLC (instrument: Waters Prep SFC100-MS; column: Chiralpak IB-N 250 mm×30 mm 5 μm; eluent: A: 40% MeOH+0.1% NH₃, B: 60% scCO₂; flow rate: 80.0 mL/min; detection: DAD; injection volume: 1.3 mL; gradient: isocratic A: 40%, B: 60%; BPR: 120 bar).

### First Eluting Stereoisomer: (7S,9S)-6-(4-(3-(benzyloxy)-4-fluoro-6-methylpicolinoyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide

181 mg, 99% pure, yield: 31%

LC-MS: Rt=1.25 min; MS m/z [M+H]⁺ 824.5/826.3, m/z [M−H]⁻ 822.1/824.1; UPLC-MS 1

LC-MS: Rt=6.29 min; MS m/z [M+H]⁺ 824.3/826.3, m/z [M−H]⁻ 822.3/824.2; UPLC-MS 2

Chiral HPLC (C-HPLC 4): Rt=0.92 min, 99% ee

### Second Eluting Stereoisomer: (7R,9R)-6-(4-(3-(benzyloxy)-4-fluoro-6-methylpicolinoyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide 175 mg, 99% pure, yield: 30%

LC-MS: Rt=1.25 min; MS m/z [M+H]⁺ 824.4/826.5, m/z [M−H]⁻ 822.1/824.1; UPLC-MS 1

LC-MS: Rt=6.29 min; MS m/z [M+H]⁺ 824.3/826.3, m/z [M−H]⁻ 822.3/824.2; UPLC-MS 2

Chiral HPLC (C-HPLC 4): Rt=3.13 min, 99% ee

### Step 2a: (7R,9R)—N-(2-chloro-4-(trifluoromethyl)phenyl)-6-(4-(4-fluoro-3-hydroxy-6-methylpicolinoyl)piperazin-1-yl)-7-methyl-2-morpholino-5-oxo-5,7,8,9-tetrahydropyrrolo[1,2-c][1,2,4]triazolo[1,5-a]pyrimidine-9-carboxamide

(7R,9R)-6-(4-(3-(benzyloxy)-4-fluoro-6-methylpicolinoyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide (the second eluting stereoisomer) (175 mg, 212 μmol) was dissolved in DCM (5 mL). TFA (5.00 mL, 64.9 mmol) was added. The mixture was stirred at 60° C. for 2.5 days. The mixture was concentrated under reduced pressure. The crude product was purified by reverse phase preparative HPLC (RP-HPLC acidic 1: 10 to 90% B in 20 min). The product containing fractions were combined, basified with aq sat NaHCO₃, extracted twice with DCM, dried through a phase separator and concentrated under reduced pressure to give the title compound (115 mg, 99% pure, yield: 73%).

The sodium salt was prepared analogous to the general procedure.

LC-MS: Rt=1.03 min; MS m/z [M+H]⁺ 734.5/735.8, m/z [M−H]⁻ 732.1/734.1; UPLC-MS 1

### Step 2b: (7S,9S)—N-(2-chloro-4-(trifluoromethyl)phenyl)-6-(4-(4-fluoro-3-hydroxy-6-methylpicolinoyl)piperazin-1-yl)-7-methyl-2-morpholino-5-oxo-5,7,8,9-tetrahydropyrrolo[1,2-c][1,2,4]triazolo[1,5-a]pyrimidine-9-carboxamide

(7S,9S)-6-(4-(3-(benzyloxy)-4-fluoro-6-methylpicolinoyl)piperazin-1-yl)-N-(2-chloro-4-(trifluoromethyl)phenyl)-7-methyl-2-morpholino-5-oxo-5,7,8,9-tetrahydropyrrolo[1,2-c][1,2,4]triazolo[1,5-a]pyrimidine-9-carboxamide (the first eluting stereoisomer) (181 mg, 219 μmol) was dissolved in DCM (5 mL). TFA (5.00 mL, 64.9 mmol) was added. The mixture was stirred at 60° C. for 1.5 days. The mixture was concentrated under reduced pressure. The crude product was purified by reverse phase preparative HPLC (RP-HPLC acidic 1: 10 to 90% B in 20 min). The product containing fractions were combined, basified with aq sat NaHCO₃, extracted twice with DCM, dried through a phase separator and concentrated under reduced pressure to give the title compound (111 mg, 99% pure, yield: 68%).

The sodium salt was prepared analogous to the general procedure.

LC-MS: Rt=1.03 min; MS m/z [M+H]⁺ 734.5/735.8, m/z [M−H]⁻ 732.1/734.1; UPLC-MS 1

The stereochemistry of example 3A as (7R,9R) was assigned based on potency and an understanding of structure-activity relationships.

### Example 4A: (7R,9R)—N-(2-chloro-6-(trifluoromethyl)pyridin-3-yl)-2-(3,6-dihydro-2H-pyran-4-yl)-6-(4-(5-hydroxy-6-methylpyrimidine-4-carbonyl)piperazin-1-yl)-7-methyl-5-oxo-5,7,8,9-tetrahydropyrrolo[1,2-c][1,2,4]triazolo[1,5-a]pyrimidine-9-carboxamide

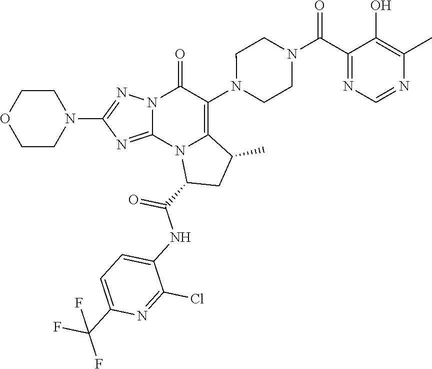 [SMILES: Cc1ncnc(C(=O)N2CCN(c3c4n(c5nc(N6CCOCC6)nn5c3=O)[C@@H](C(=O)Nc3ccc(C(F)(F)F)nc3Cl)C[C@H]4C)CC2)c1O]

### Step 1: (7R,9R)-6-(4-(5-(benzyloxy)-6-methylpyrimidine-4-carbonyl)piperazin-1-yl)-N-(2-chloro-6-(trifluoromethyl)pyridin-3-yl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide

To (7R,9R)—N-(2-chloro-6-(trifluoromethyl)pyridin-3-yl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide (Intermediate E1) (127 mg, 206 μmol) and 5-(benzyloxy)-6-methylpyrimidine-4-carboxylic acid (Intermediate U) (50.4 mg, 206 μmol) in DMF (1 mL) was added DIPEA (180 μL, 1.03 mmol). The RM was stirred at RT for 2 minutes then HATU (86.0 mg, 227 μmol) was added and the RM was stirred at RT for 13 minutes. The RM was diluted with water (3 mL) and sonicated. The resulting suspension was stirred for 1 hour. The mixture was filtered and dried to give the title compound as a colourless solid (114 mg, 90% pure, yield: 62%).

LC-MS: Rt=1.08 min; MS m/z [M+H−Boc]⁺805.5/807.5, m/z [M−H]⁻ 803.4/805.4; UPLC-MS 1

### Step 2: (7R,9R)—N-(2-chloro-6-(trifluoromethyl)pyridin-3-yl)-2-(3,6-dihydro-2H-pyran-4-yl)-6-(4-(5-hydroxy-6-methylpyrimidine-4-carbonyl)piperazin-1-yl)-7-methyl-5-oxo-5,7,8,9-tetrahydropyrrolo[1,2-c][1,2,4]triazolo[1,5-a]pyrimidine-9-carboxamide

(7R,9R)-6-(4-(5-(benzyloxy)-6-methylpyrimidine-4-carbonyl)piperazin-1-yl)-N-(2-chloro-6-(trifluoromethyl)pyridin-3-yl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide (114 mg, 142 μmol) was dissolved in TFA (1.00 mL, 13.0 mmol) and stirred at 55° C. for 5.5 hours. The RM was evaporated and the crude product was purified by reverse phase preparative HPLC (RP-HPLC acidic 1: 20 to 50% B in 15 min). The product containing fractions were combined and lyophilized. The resulting powder (TFA solvate) was redissolved in DCM (5 mL) and solid NaHCO₃ (50.0 mg) was added. The suspension was kept at RT for 20 minutes, filtered and evaporated under reduced pressure to give the title compound as a colourless solid (62.0 mg, 99% pure, yield: 61%).

The sodium salt was prepared analogous to the general procedure.

LC-MS: Rt=0.91 min; MS m/z [M+H−Boc]⁺ 715.4/717.4, m/z [M−H]⁻ 713.1/715.1; UPLC-MS 1

The stereochemistry of example 4A as (7R,9R) was assigned based on its potency and an understanding of structure-activity relationships.

### Example 4B: (7S,9S)—N-(2-chloro-6-(trifluoromethyl)pyridin-3-yl)-2-(3,6-dihydro-2H-pyran-4-yl)-6-(4-(5-hydroxy-6-methylpyrimidine-4-carbonyl)piperazin-1-yl)-7-methyl-5-oxo-5,7,8,9-tetrahydropyrrolo[1,2-c][1,2,4]triazolo[1,5-a]pyrimidine-9-carboxamide

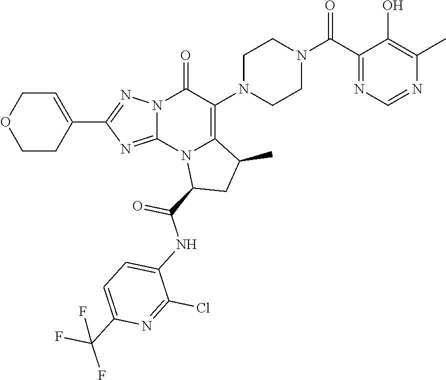 [SMILES: Cc1ncnc(C(=O)N2CCN(c3c4n(c5nc(C6=CCOCC6)nn5c3=O)[C@H](C(=O)Nc3ccc(C(F)(F)F)nc3Cl)C[C@@H]4C)CC2)c1O]

### Step 1: (7S,9S)-6-(4-(5-(benzyloxy)-6-methylpyrimidine-4-carbonyl)piperazin-1-yl)-N-(2-chloro-6-(trifluoromethyl)pyridin-3-yl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide

To (7S,9S)—N-(2-chloro-6-(trifluoromethyl)pyridin-3-yl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide (Intermediate E2) (122 mg, 198 μmol) and 5-(benzyloxy)-6-methylpyrimidine-4-carboxylic acid (Intermediate U) (48.0 mg, 196 μmol) in DMF (1 mL) was added DIPEA (180 μL, 1.03 mmol). The RM was stirred at RT for 2 minutes then HATU (82.0 mg, 216 μmol) was added and the RM was stirred at RT for 13 minutes. The RM was diluted with water (3 mL) and sonicated. The resulting suspension was stirred for 1 hour. The mixture was filtered and dried to give the title compound as an off-white solid (104 mg, 85% pure, yield: 53%).

LC-MS: Rt=1.10 min; MS m/z [M+H−Boc]⁺ 805.5/807.6, m/z [M−H]⁻ 803.2/805.2; UPLC-MS 1

### Step 2: (7S,9S)—N-(2-chloro-6-(trifluoromethyl)pyridin-3-yl)-2-(3,6-dihydro-2H-pyran-4-yl)-6-(4-(5-hydroxy-6-methylpyrimidine-4-carbonyl)piperazin-1-yl)-7-methyl-5-oxo-5,7,8,9-tetrahydropyrrolo[1,2-c][1,2,4]triazolo[1,5-a]pyrimidine-9-carboxamide

(7S,9S)-6-(4-(5-(benzyloxy)-6-methylpyrimidine-4-carbonyl)piperazin-1-yl)-N-(2-chloro-6-(trifluoromethyl)pyridin-3-yl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide (104 mg, 129 μmol) was dissolved in TFA (1.00 mL, 13.0 mmol) and stirred at 55° C. for 5.5 hours. The RM was evaporated and the crude product was purified by reverse phase preparative HPLC (RP-HPLC acidic 1: 20 to 50% B in 15 min). The product containing fractions were combined and lyophilized. The resulting powder (TFA solvate) was redissolved in DCM (5 mL) and solid NaHCO₃ (50.0 mg) was added. The suspension was kept at RT for 20 minutes, filtered and concentrated under reduced pressure to give the title compound as a colourless solid (51.0 mg, 99% pure, yield: 50%).

LC-MS: Rt=0.91 min; MS m/z [M+H−Boc]⁺ 715.4/717.4, m/z [M−H]⁻ 713.1/715.1; UPLC-MS 1

### Example 5A: (7R,9R)—N-(2-chloro-4-(trifluoromethyl)phenyl)-2-(3,6-dihydro-2H-pyran-4-yl)-6-(4-(7-hydroxy-2,3-dihydrofuro[3,2-c]pyridine-6-carbonyl)piperazin-1-yl)-7-methyl-5-oxo-5,7,8,9-tetrahydropyrrolo[1,2-c][1,2,4]triazolo[1,5-a]pyrimidine-9-carboxamide

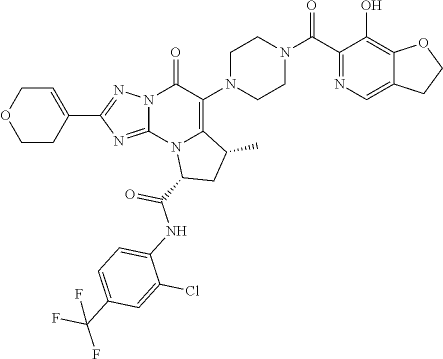 [SMILES: C[C@@H]1C[C@H](C(=O)Nc2ccc(C(F)(F)F)cc2Cl)n2c1c(N1CCN(C(=O)c3ncc4c(c3O)OCC4)CC1)c(=O)n1nc(C3=CCOCC3)nc21]

### Step 1: (7R,9R)—N-(2-chloro-4-(trifluoromethyl)phenyl)-2-(3,6-dihydro-2H-pyran-4-yl)-6-(4-(7-(methoxymethoxy)-2,3-dihydrofuro[3,2-c]pyridine-6-carbonyl)piperazin-1-yl)-7-methyl-5-oxo-5,7,8,9-tetrahydropyrrolo[1,2-c][1,2,4]triazolo[1,5-a]pyrimidine-9-carboxamide

To a stirred solution of (7R,9R)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide (Intermediate H4) (155 mg, 268 μmol), 7-(methoxymethoxy)-2,3-dihydrofuro[3,2-c]pyridine-6-carboxylic acid (Intermediate Z) (60.4 mg, 268 μmol) and HATU (122 mg, 322 μmol) in DMF (2 mL) was added DIPEA (234 μL, 1.34 mmol) at RT, and the RM was stirred at RT for 2 minutes. The RM was diluted with EtOAc and water, extracted twice with EtOAc and the combined organic layers were dried over Na₂SO₄ and concentrated under reduced pressure. The crude product was purified by column chromatography (RediSep Column: Silica 12 g, eluent DCM:DCM/MeOH (8/2) 100:0 to 50:50). The product containing fractions were combined, concentrated under reduced pressure and dried under HV to give the title compound (164 mg, 96% pure, yield: 75%).

LC-MS: Rt=1.03 min; MS m/z [M+H]⁺ 785.5/787.5, m/z [M−H]⁻ 783.4/784.7; UPLC-MS 1

LC-MS: Rt=5.04 min; MS m/z [M+H]⁺ 785.5/787.5, m/z [M−H]⁻ 783.3/785.3; UPLC-MS 2

### Step 2: (7R,9R)—N-(2-chloro-4-(trifluoromethyl)phenyl)-2-(3,6-dihydro-2H-pyran-4-yl)-6-(4-(7-hydroxy-2,3-dihydrofuro[3,2-c]pyridine-6-carbonyl)piperazin-1-yl)-7-methyl-5-oxo-5,7,8,9-tetrahydropyrrolo[1,2-c][1,2,4]triazolo[1,5-a]pyrimidine-9-carboxamide

(7R,9R)—N-(2-chloro-4-(trifluoromethyl)phenyl)-2-(3,6-dihydro-2H-pyran-4-yl)-6-(4-(7-(methoxymethoxy)-2,3-dihydrofuro[3,2-c]pyridine-6-carbonyl)piperazin-1-yl)-7-methyl-5-oxo-5,7,8,9-tetrahydropyrrolo[1,2-c][1,2,4]triazolo[1,5-a]pyrimidine-9-carboxamide (164 mg, 201 μmol) in EtOH (1.5 mL) and 1.25 M HCl in EtOH (1.50 mL, 1.88 mmol) was stirred at RT for 14 hours. The RM was diluted with DCM and aq NaHCO₃, extracted twice with DCM and the combined organic layers were dried over Na₂SO₄ and concentrated under reduced pressure. The crude product was purified by reverse phase preparative HPLC (RP-HPLC acidic 1: 20 to 75% B in 20 min) to give the title compound as a white solid (117 mg, 100% pure, yield: 79%).

The sodium salt was prepared analogous to the general procedure.

LC-MS: Rt=0.94 min; MS m/z [M+H]⁺ 741.6/743.6, m/z [M−H]⁻ 739.4/741.4; UPLC-MS 1

LC-MS: Rt=4.75 min; MS m/z [M+H]⁺ 741.5/743.5, m/z [M−H]⁻ 739.5/741.4; UPLC-MS 2

The stereochemistry of example 5A as (7R,9R) was assigned based on its potency and an understanding of structure-activity relationships.

### Example 5B: (7S,9S)—N-(2-chloro-4-(trifluoromethyl)phenyl)-2-(3,6-dihydro-2H-pyran-4-yl)-6-(4-(7-hydroxy-2,3-dihydrofuro[3,2-c]pyridine-6-carbonyl)piperazin-1-yl)-7-methyl-5-oxo-5,7,8,9-tetrahydropyrrolo[1,2-c][1,2,4]triazolo[1,5-a]pyrimidine-9-carboxamide

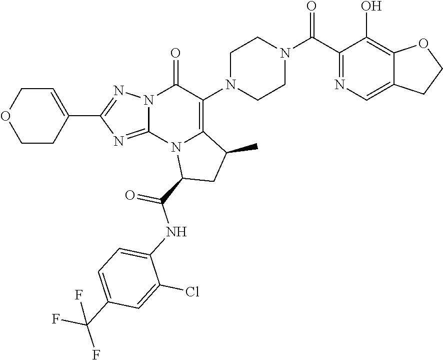 [SMILES: C[C@H]1C[C@@H](C(=O)Nc2ccc(C(F)(F)F)cc2Cl)n2c1c(N1CCN(C(=O)c3ncc4c(c3O)OCC4)CC1)c(=O)n1nc(C3=CCOCC3)nc21]

### Step 1: (7S,9S)—N-(2-chloro-4-(trifluoromethyl)phenyl)-2-(3,6-dihydro-2H-pyran-4-yl)-6-(4-(7-(methoxymethoxy)-2,3-dihydrofuro[3,2-c]pyridine-6-carbonyl)piperazin-1-yl)-7-methyl-5-oxo-5,7,8,9-tetrahydropyrrolo[1,2-c][1,2,4]triazolo[1,5-a]pyrimidine-9-carboxamide

To a stirred solution of (7S,9S)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide (Intermediate H3) (150 mg, 260 μmol), 7-(methoxymethoxy)-2,3-dihydrofuro[3,2-c]pyridine-6-carboxylic acid (Intermediate Z) (58.4 mg, 260 μmol) and HATU (118 mg, 311 μmol) in DMF (2 mL) was added DIPEA (227 μL, 1.30 mmol) at RT, and the RM was stirred at RT for 2 minutes. The RM was diluted with EtOAc and water, extracted twice with EtOAc and the combined organic layer was dried over Na₂SO₄ and concentrated under reduced pressure. The crude product was purified by column chromatography (RediSep Column: Silica 24 g, eluent DCM:DCM/MeOH (8/2) 100:0 to 50:50). The product containing fractions were combined, concentrated under reduced pressure and dried under HV to give the title compound (155 mg, 95% pure, yield: 72%).

LC-MS: Rt=1.02 min; MS m/z [M+H]⁺ 785.5/787.5, m/z [M−H]⁻ 783.3/785.3; UPLC-MS 1

LC-MS: Rt=5.05 min; MS m/z [M+H]⁺ 785.6/787.5, m/z [M−H]⁻ 783.4/785.3; UPLC-MS 2

### Step 2: (7S,9S)—N-(2-chloro-4-(trifluoromethyl)phenyl)-2-(3,6-dihydro-2H-pyran-4-yl)-6-(4-(7-hydroxy-2,3-dihydrofuro[3,2-c]pyridine-6-carbonyl)piperazin-1-yl)-7-methyl-5-oxo-5,7,8,9-tetrahydropyrrolo[1,2-c][1,2,4]triazolo[1,5-a]pyrimidine-9-carboxamide

(7S,9S)—N-(2-chloro-4-(trifluoromethyl)phenyl)-2-(3,6-dihydro-2H-pyran-4-yl)-6-(4-(7-(methoxymethoxy)-2,3-dihydrofuro[3,2-c]pyridine-6-carbonyl)piperazin-1-yl)-7-methyl-5-oxo-5,7,8,9-tetrahydropyrrolo[1,2-c][1,2,4]triazolo[1,5-a]pyrimidine-9-carboxamide (155 mg, 188 μmol) in EtOH (1.5 mL) and 1.25 M HCl in EtOH (1.50 mL, 1.88 mmol) was stirred at RT for 14 hours. The RM was diluted with DCM and aq NaHCO₃, extracted twice with DCM and the combined organic layer was dried over Na₂SO₄ and concentrated under reduced pressure. The crude product was purified by reverse phase preparative HPLC (RP-HPLC acidic 1: 5 to 95% B in 25 min) to give the title compound as a white solid (111 mg, 99% pure, yield: 79%).

The sodium salt was prepared analogous to the general procedure.

LC-MS: Rt=0.93 min; MS m/z [M+H]⁺ 741.6/743.4, m/z [M−H]⁻ 739.4/741.4; UPLC-MS 1

LC-MS: Rt=4.67 min; MS m/z [M+H]⁺ 741.6/743.6, m/z [M−H]⁻ 739.4/741.3; UPLC-MS 2

### Example 6A: (7R,9R)—N-(2-chloro-4-(trifluoromethyl)phenyl)-2-(3,6-dihydro-2H-pyran-4-yl)-6-(4-(4-fluoro-3-hydroxypicolinoyl)piperazin-1-yl)-7-methyl-5-oxo-5,7,8,9-tetrahydropyrrolo[1,2-c][1,2,4]triazolo[1,5-a]pyrimidine-9-carboxamide

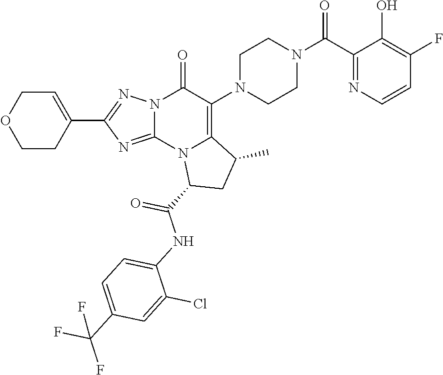 [SMILES: C[C@@H]1C[C@H](C(=O)Nc2ccc(C(F)(F)F)cc2Cl)n2c1c(N1CCN(C(=O)c3nccc(F)c3O)CC1)c(=O)n1nc(C3=CCOCC3)nc21]

### Step 1: (7R,9R)-6-(4-(3-(benzyloxy)-4-fluoropicolinoyl)piperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide

To a stirred solution of (7R,9R)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide (Intermediate H4) (155 mg, 268 μmol), 3-(benzyloxy)-4-fluoropicolinic acid (Intermediate W) (66.3 mg, 268 μmol) and HATU (122 mg, 322 μmol) in DMF (2 mL) was added DIPEA (234 μL, 1.34 mmol) at RT, and the RM was stirred at RT for 3 minutes. The RM was diluted with EtOAc and water, extracted twice with EtOAc and the combined organic layers were dried over Na₂SO₄ and concentrated under reduced pressure. The crude product was purified by column chromatography (RediSep Column: Silica 12 g, eluent DCM:DCM/MeOH (8/2) 100:0 to 50:50). The product containing fractions were combined, concentrated under reduced pressure and dried under HV to give the title compound (211 mg, 94% pure, yield: 92%).

LC-MS: Rt=1.23 min; MS m/z [M+H]⁺ 807.6/809.5, m/z [M−H]⁻ 805.4/807.4; UPLC-MS 1

LC-MS: Rt=6.19 min; MS m/z [M+H]⁺ 807.5/809.5, m/z [M−H]⁻ 805.6/807.4; UPLC-MS 2

### Step 2: (7R,9R)—N-(2-chloro-4-(trifluoromethyl)phenyl)-2-(3,6-dihydro-2H-pyran-4-yl)-6-(4-(4-fluoro-3-hydroxypicolinoyl)piperazin-1-yl)-7-methyl-5-oxo-5,7,8,9-tetrahydropyrrolo[1,2-c][1,2,4]triazolo[1,5-a]pyrimidine-9-carboxamide

(7R,9R)-6-(4-(3-(benzyloxy)-4-fluoropicolinoyl)piperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide (210 mg, 245 μmol) in TFA (2.00 mL, 26.0 mmol) was stirred at 50° C. for 14 hours. The RM was diluted with DCM and aq NaHCO₃, extracted twice with DCM and the combined organic layers were dried over Na₂SO₄ and concentrated under reduced pressure. The crude product was purified by reverse phase preparative HPLC (RP-HPLC acidic 1: 20 to 75% B in 20 min) to give the title compound as a white solid (96.0 mg, 100% pure, yield: 55%).

The sodium salt was prepared analogous to the general procedure.

LC-MS: Rt=1.02 min; MS m/z [M+H]⁺ 717.5/719.5, m/z [M−H]⁻ 715.3/717.3; UPLC-MS 1

LC-MS: Rt=5.04 min; MS m/z [M+H]⁺ 717.5/719.5, m/z [M−H]⁻ 715.3/717.3; UPLC-MS 2

The stereochemistry of example 6A as (7R,9R) was assigned based on its potency and an understanding of structure-activity relationships.

### Example 6B: (7S,9S)—N-(2-chloro-4-(trifluoromethyl)phenyl)-2-(3,6-dihydro-2H-pyran-4-yl)-6-(4-(4-fluoro-3-hydroxypicolinoyl)piperazin-1-yl)-7-methyl-5-oxo-5,7,8,9-tetrahydropyrrolo[1,2-c][1,2,4]triazolo[1,5-a]pyrimidine-9-carboxamide

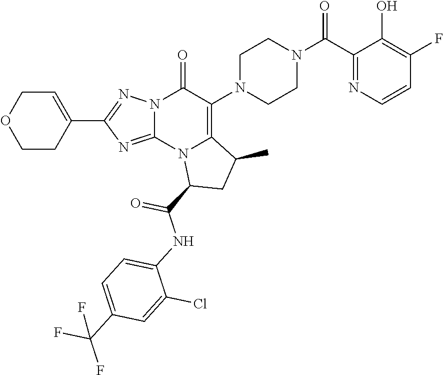 [SMILES: C[C@H]1C[C@@H](C(=O)Nc2ccc(C(F)(F)F)cc2Cl)n2c1c(N1CCN(C(=O)c3nccc(F)c3O)CC1)c(=O)n1nc(C3=CCOCC3)nc21]

### Step 1: (7S,9S)-6-(4-(3-(benzyloxy)-4-fluoropicolinoyl)piperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide

To a stirred solution of (7S,9S)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide (Intermediate H3) (150 mg, 260 μmol), 3-(benzyloxy)-4-fluoropicolinic acid (Intermediate W) (64.2 mg, 260 μmol) and HATU (118 mg, 311 μmol) in DMF (2 mL) was added DIPEA (227 μL, 1.30 mmol) at RT, and the RM was stirred at RT for 10 minutes. The RM was diluted with EtOAc and water, extracted twice with EtOAc and the combined organic layers were dried over Na₂SO₄ and concentrated under reduced pressure. The crude product was purified by column chromatography (RediSep Column: Silica 12 g, eluent DCM:DCM/MeOH (8/2) 100:0 to 50:50). The product containing fractions were combined, concentrated under reduced pressure and dried under HV to give the title compound (159 mg, 92% pure, yield: 70%).

LC-MS: Rt=1.22 min; MS m/z [M+H]⁺ 807.5/809.5, m/z [M−H]⁻ 805.4/807.4; UPLC-MS 1

LC-MS: Rt=6.09 min; MS m/z [M+H]⁺ 807.5/809.5, m/z [M−H]⁻ 805.4/807.4; UPLC-MS 2

### Step 2: (7S,9S)—N-(2-chloro-4-(trifluoromethyl)phenyl)-2-(3,6-dihydro-2H-pyran-4-yl)-6-(4-(4-fluoro-3-hydroxypicolinoyl)piperazin-1-yl)-7-methyl-5-oxo-5,7,8,9-tetrahydropyrrolo[1,2-c][1,2,4]triazolo[1,5-a]pyrimidine-9-carboxamide

(7S,9S)-6-(4-(3-(benzyloxy)-4-fluoropicolinoyl)piperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide (159 mg, 181 μmol) in TFA (2.00 mL, 26.0 mmol) was stirred at 50° C. for 14 hours. The RM was diluted with DCM and aq NaHCO₃, extracted twice with DCM and the combined organic layers were dried over Na₂SO₄ and concentrated under reduced pressure. The crude product was purified by reverse phase preparative HPLC (RP-HPLC acidic 1: 20 to 75% B in 20 min) to give the title compound as a white solid (77.0 mg, 98% pure, yield: 58%).

The sodium salt was prepared analogous to the general procedure.

LC-MS: Rt=1.02 min; MS m/z [M+H]⁺ 716.9/718.9, m/z [M−H]⁻ 715.3/717.4; UPLC-MS 1

LC-MS: Rt=5.06 min; MS m/z [M+H]⁺ 716.9/718.8, m/z [M−H]⁻ 715.4/717.3; UPLC-MS 2

### Example 7: (7R,9R)—N-(2-chloro-4-(trifluoromethyl)phenyl)-2-(3,6-dihydro-2H-pyran-4-yl)-7-methyl-6-(4-(1-methyl-5-oxo-4,5-dihydro-1H-1,2,4-triazole-3-carbonyl)piperazin-1-yl)-5-oxo-5,7,8,9-tetrahydropyrrolo[1,2-c][1,2,4]triazolo[1,5-a]pyrimidine-9-carboxamide

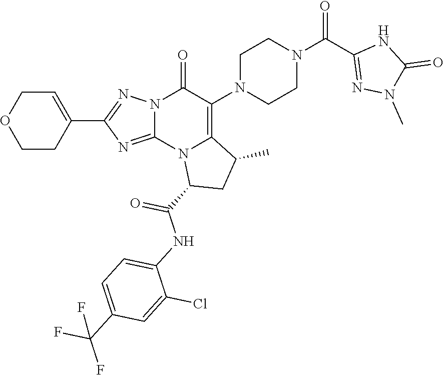 [SMILES: C[C@@H]1C[C@H](C(=O)Nc2ccc(C(F)(F)F)cc2Cl)n2c1c(N1CCN(C(=O)c3nn(C)c(=O)[nH]3)CC1)c(=O)n1nc(C3=CCOCC3)nc21]

1-Methyl-5-oxo-4,5-dihydro-1H-1,2,4-triazole-3-carboxylic acid (20.9 mg, 138 μmol) was suspended in DCM (1.4 mL) at RT under argon. 1-Chloro-N,N,2-trimethylprop-1-en-1-amine (21.9 mg, 152 μmol) was added and the RM was stirred at RT for 1.75 hours. (7R,9R)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide (Intermediate H4) (40.0 mg, 69.0 μmol) was added, followed by DIPEA (61.0 μL, 346 μmol), and the resulting brown solution was stirred at RT for 1 hour. Water (3 mL) and aq sat NaHCO₃ (2 mL) were added and the RM was kept at RT overnight. The RM was extracted with DCM (4×20 mL), and the combined organic layers were washed with water (5 mL), dried through a phase separator and concentrated under reduced pressure to give an orange solid (66.0 mg). The crude product was purified by reverse phase preparative HPLC (RP-HPLC acidic 1: 15 to 85% B in 20 min). The product containing fractions were combined, ACN was removed under reduced pressure and the aqueous residue was basified with aq sat NaHCO₃ and extracted with DCM (4×20 mL). The combined organic layers were dried through a phase separator and concentrated under reduced pressure to give the title compound as a white solid (35.8 mg, 99% pure, yield: 73%).

The sodium salt was prepared analogous to the general procedure.

LC-MS: Rt=0.99 min; MS m/z [M+H]⁺ 703.6/705.6, m/z [M−H]⁻ 701.3/703.3; UPLC-MS 1

LC-MS: Rt=4.89 min; MS m/z [M+H]⁺ 703.1/705.1, m/z [M−H]⁻ 701.3/703.3; UPLC-MS 2

### Example 8: (7R,9R)—N-(2-chloro-4-(trifluoromethyl)phenyl)-2-(3,6-dihydro-2H-pyran-4-yl)-6-(4-(4-fluoro-3-hydroxy-6-methylpicolinoyl)piperazin-1-yl)-7-methyl-5-oxo-5,7,8,9-tetrahydropyrrolo[1,2-c][1,2,4]triazolo[1,5-a]pyrimidine-9-carboxamide

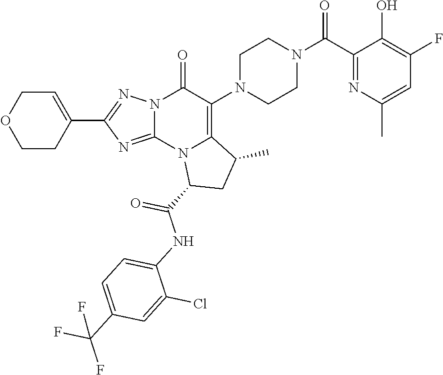 [SMILES: Cc1cc(F)c(O)c(C(=O)N2CCN(c3c4n(c5nc(C6=CCOCC6)nn5c3=O)[C@@H](C(=O)Nc3ccc(C(F)(F)F)cc3Cl)C[C@H]4C)CC2)n1]

### Step 1: (7R,9R)-6-(4-(3-(benzyloxy)-4-fluoro-6-methylpicolinoyl)piperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide

To a stirred solution of (7R,9R)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide (Intermediate H4) (155 mg, 268 μmol), 3-(benzyloxy)-4-fluoro-6-methylpicolinic acid (Intermediate V) (80.0 mg, 268 μmol) and HATU (122 mg, 322 μmol) in DMF (2 mL) was added DIPEA (234 μL, 1.34 mmol) at RT, and the RM was stirred at RT for 5 minutes. The RM was diluted with EtOAc and water, extracted twice with EtOAc and the combined organic layers were dried over Na₂SO₄ and concentrated under reduced pressure. The crude product was purified by column chromatography (RediSep Column: Silica 12 g, eluent DCM:DCM/MeOH (8/2) 100:0 to 50:50). The product containing fractions were combined, concentrated under reduced pressure and dried under HV to give the title compound as a white foam (220 mg, 92% pure, yield: 92%).

LC-MS: Rt=1.28 min; MS m/z [M+H]⁺ 821.6/823.6, m/z [M−H]⁻ 819.5/821.4; UPLC-MS 1

LC-MS: Rt=6.43 min; MS m/z [M+H]⁺ 821.5/823.6, m/z [M−H]⁻ 819.6/821.6; UPLC-MS 2

### Step 2: (7R,9R)—N-(2-chloro-4-(trifluoromethyl)phenyl)-2-(3,6-dihydro-2H-pyran-4-yl)-6-(4-(4-fluoro-3-hydroxy-6-methylpicolinoyl)piperazin-1-yl)-7-methyl-5-oxo-5,7,8,9-tetrahydropyrrolo[1,2-c][1,2,4]triazolo[1,5-a]pyrimidine-9-carboxamide

(7R,9R)-6-(4-(3-(benzyloxy)-4-fluoro-6-methylpicolinoyl)piperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide (220 mg, 246 μmol) in TFA (2.00 mL, 26.0 mmol) was stirred at 50° C. for 40 hours. The RM was diluted with DCM and aq NaHCO₃, extracted twice with DCM and the combined organic layers were dried over Na₂SO₄ and concentrated under reduced pressure. The crude product was purified by column chromatography (RediSep Column: Silica 24 g, eluent DCM:DCM/MeOH (8/2) 100:0 to 50:50). The product containing fractions were combined, concentrated under reduced pressure and dried under HV to give the title compound as a white solid (150 mg, 100% pure, yield: 83%).

The sodium salt was prepared analogous to the general procedure.

LC-MS: Rt=1.06 min; MS m/z [M+H]⁺ 731.5/733.5, m/z [M−H]⁻ 729.3/731.3; UPLC-MS 1

LC-MS: Rt=5.25 min; MS m/z [M+H]⁺ 731.6/733.5, m/z [M−H]⁻ 729.3/731.4; UPLC-MS 2

### Example 9: (7R,9R)—N-(2-chloro-4-(trifluoromethyl)phenyl)-2-(3,6-dihydro-2H-pyran-4-yl)-6-(4-(6-hydroxyimidazo[1,2-a]pyridine-5-carbonyl)piperazin-1-yl)-7-methyl-5-oxo-5,7,8,9-tetrahydropyrrolo[1,2-c][1,2,4]triazolo[1,5-a]pyrimidine-9-carboxamide

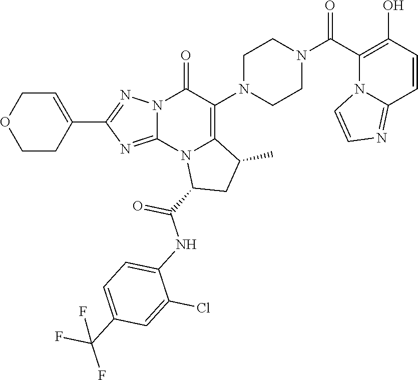 [SMILES: C[C@@H]1C[C@H](C(=O)Nc2ccc(C(F)(F)F)cc2Cl)n2c1c(N1CCN(C(=O)c3c(O)ccc4nccn34)CC1)c(=O)n1nc(C3=CCOCC3)nc21]

### Step 1: (7R,9R)-6-(4-(6-(benzyloxy)imidazo[1,2-a]pyridine-5-carbonyl)piperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide

(7R,9R)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide (Intermediate H4) (71.1 mg, 123 μmol) and 6-(benzyloxy)imidazo[1,2-a]pyridine-5-carboxylic acid (Intermediate AA) (33.0 mg, 123 μmol) were mixed in DMF (1.5 mL) at RT under argon. HATU (56.1 mg, 148 μmol) and DIPEA (107 μL, 615 μmol) were added and the RM was stirred at RT for 45 minutes. Then it was quenched with water (10 mL). The RM was extracted with EtOAc (3×50 mL), washed with water (3×10 mL) and brine (2×10 mL). The combined organic layers were dried through a phase separator with Na₂SO₄ and concentrated under reduced pressure to give a brown oil (161 mg). The crude product was purified by reverse phase preparative HPLC (RP-HPLC acidic 1: 20 to 80% B in 20 min). The product containing fractions were combined, basified with aq sat NaHCO₃, ACN was removed under reduced pressure and the aqueous residue was extracted with DCM (4×20 mL). The combined organic layers were dried through a phase separator and concentrated under reduced pressure to give the title compound as a pale pink solid (63.0 mg, 99% pure, yield: 61%).

LC-MS: Rt=1.04 min; MS m/z [M+H]⁺ 828.3/830.4, m/z [M−H]⁻ 826.3/828.3; UPLC-MS 1

### Step 2: (7R,9R)—N-(2-chloro-4-(trifluoromethyl)phenyl)-2-(3,6-dihydro-2H-pyran-4-yl)-6-(4-(6-hydroxyimidazo[1,2-a]pyridine-5-carbonyl)piperazin-1-yl)-7-methyl-5-oxo-5,7,8,9-tetrahydropyrrolo[1,2-c][1,2,4]triazolo[1,5-a]pyrimidine-9-carboxamide

(7R,9R)-6-(4-(6-(benzyloxy)imidazo[1,2-a]pyridine-5-carbonyl)piperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide (61.5 mg, 74.0 μmol) was mixed with TFA (1.00 mL, 13.0 mmol) and the RM was stirred at 50° C. for 52.5 hours, then at RT for 2 days. The RM was concentrated under reduced pressure and dried under HV. The brown solid residue was extracted with DCM (4×50 mL), washed with aq sat NaHCO₃ (15 mL) and brine (15 mL). The combined organic layers were dried through a phase separator and concentrated under reduced pressure to give a bright brown solid (77.0 mg). The crude product was adsorbed onto Isolute and purified by column chromatography (RediSep Column: Silica 12 g, eluent DCM:DCM/MeOH (8/2) 100:0 to 20:80). The product containing fractions were combined and concentrated under reduced pressure to give the title compound as a slightly beige solid (35.4 mg, 100% pure, yield: 65%).

The sodium salt was prepared analogous to the general procedure.

LC-MS: Rt=0.88 min; MS m/z [M+H]⁺ 738.6/740.7, m/z [M−H]⁻ 736.4/738.3; UPLC-MS 1

LC-MS: Rt=4.35 min; MS m/z [M+H]⁺ 738.2/740.2, m/z [M−H]⁻ 736.3/738.2; UPLC-MS 2

### Example 10: (7R,9R)—N-(2-chloro-4-(trifluoromethyl)phenyl)-2-(3,6-dihydro-2H-pyran-4-yl)-6-(4-(3-hydroxy-2-methylisonicotinoyl)piperazin-1-yl)-7-methyl-5-oxo-5,7,8,9-tetrahydropyrrolo[1,2-c][1,2,4]triazolo[1,5-a]pyrimidine-9-carboxamide

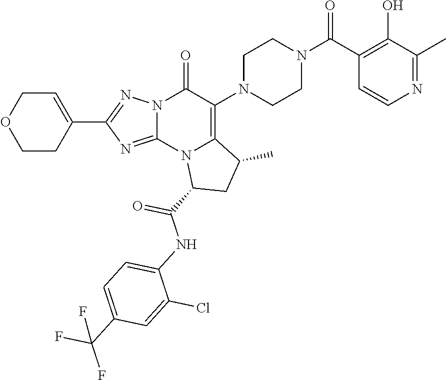 [SMILES: Cc1nccc(C(=O)N2CCN(c3c4n(c5nc(C6=CCOCC6)nn5c3=O)[C@@H](C(=O)Nc3ccc(C(F)(F)F)cc3Cl)C[C@H]4C)CC2)c1O]

3-Hydroxy-2-methylisonicotinic acid (41.4 mg, 208 μmol) was dissolved in DCM (2 mL) at RT under argon. 1-Chloro-N,N,2-trimethylprop-1-en-1-amine (32.8 mg, 228 μmol) was added and the RM was stirred at RT for 2.25 hours. (7R,9R)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide (Intermediate H4) (60.0 mg, 104 μmol) and DIPEA (91.0 μL, 519 μmol) were added and the RM was stirred at RT for 4 days. The RM was quenched with water (2 mL) and aq sat NaHCO₃ (2 mL) and extracted with DCM (4×20 mL). The combined organic layers were washed with water (5 mL), dried through a phase separator and concentrated under reduced pressure to give a brown oil (126 mg). The crude product was purified by reverse phase preparative HPLC (RP-HPLC acidic 1: 20 to 80% B in 20 min). The product containing fractions were combined, basified with aq sat NaHCO₃, ACN was removed under reduced pressure and the aqueous residue was extracted with DCM (4×20 mL). The combined organic layers were washed with water, dried through a phase separator and concentrated under reduced pressure to give the title compound as a white solid (37.0 mg, 100% pure, yield: 50%).

The sodium salt was prepared analogous to the general procedure.

LC-MS: Rt=0.95 min; MS m/z [M+H]⁺ 713.5/715.5, m/z [M−H]⁻ 711.2/713.2; UPLC-MS 1

### Example 11: (7R,9R)—N-(2-chloro-4-(trifluoromethyl)phenyl)-2-(3,6-dihydro-2H-pyran-4-yl)-6-(4-(3-hydroxypicolinoyl)piperazin-1-yl)-7-methyl-5-oxo-5,7,8,9-tetrahydropyrrolo[1,2-c][1,2,4]triazolo[1,5-a]pyrimidine-9-carboxamide

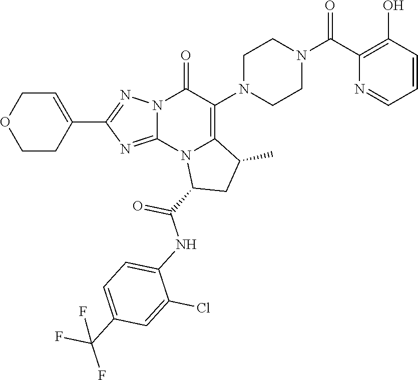 [SMILES: C[C@@H]1C[C@H](C(=O)Nc2ccc(C(F)(F)F)cc2Cl)n2c1c(N1CCN(C(=O)c3ncccc3O)CC1)c(=O)n1nc(C3=CCOCC3)nc21]

(7R,9R)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide (Intermediate H4) (79.0 mg, 137 μmol) was dissolved in DCM (1.4 mL) at 0° C. under argon. 3-Hydroxypicolinoyl chloride (Intermediate X) (36.6 mg, 232 μmol) was added, followed by slow addition of DIPEA (120 μL, 683 μmol). The RM was stirred at RT for 1.75 hours, 3-hydroxypicolinoyl chloride (Intermediate X) (15.0 mg, 95.0 μmol) was added again and the RT was stirred at RT for 4.5 hours. The RM was quenched with water (3 mL) and aq sat NaHCO₃ (3 mL) and extracted with DCM (4×15 mL). The combined organic layers were dried through a phase separator and concentrated under reduced pressure to give a brown solid (117 mg). The crude product was purified by reverse phase preparative HPLC (RP-HPLC acidic 1: 10 to 70% B in 20 min). The product containing fractions were combined, basified with aq sat NaHCO₃, ACN was removed under reduced pressure and the aqueous residue was extracted with DCM (4×15 mL). The aqueous layer was extracted twice with EtOAc. The combined organic layers were dried through a phase separator and concentrated under reduced pressure to give the title compound as a beige solid (57.0 mg, 100% pure, yield: 60%).

The sodium salt was prepared analogous to the general procedure.

LC-MS: Rt=1.01 min; MS m/z [M+H]⁺ 699.5/701.3, m/z [M−H]⁻ 697.1/699.1; UPLC-MS 1

LC-MS: Rt=5.08 min; MS m/z [M+H]⁺ 699.2/701.2, m/z [M−H]⁻ 697.2/699.2; UPLC-MS 2

### Example 12: (7R,9R)—N-(2-chloro-4-(trifluoromethyl)phenyl)-2-(3,6-dihydro-2H-pyran-4-yl)-6-(4-(2-fluoro-3-hydroxyisonicotinoyl)piperazin-1-yl)-7-methyl-5-oxo-5,7,8,9-tetrahydropyrrolo[1,2-c][1,2,4]triazolo[1,5-a]pyrimidine-9-carboxamide

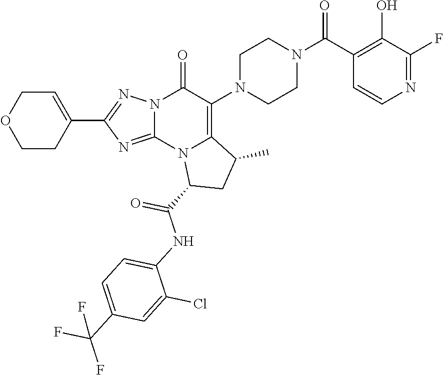 [SMILES: C[C@@H]1C[C@H](C(=O)Nc2ccc(C(F)(F)F)cc2Cl)n2c1c(N1CCN(C(=O)c3ccnc(F)c3O)CC1)c(=O)n1nc(C3=CCOCC3)nc21]

2-Fluoro-3-hydroxyisonicotinic acid (54.4 mg, 346 μmol) was dissolved in DCM (3.5 mL) at RT under argon. 1-Chloro-N,N,2-trimethylprop-1-en-1-amine (54.4 mg, 346 μmol) was added and the RM was stirred at RT for 2.25 hours. (7R,9R)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide (Intermediate H4) (100 mg, 173 μmol) was added, followed by DIPEA (151 μL, 865 μmol). The RM was stirred at RT for 50 minutes. The RM was quenched with water (3 mL) and aq sat NaHCO₃ (3 mL) and extracted with DCM (4×20 mL). The combined organic layers were dried through a phase separator and concentrated under reduced pressure to give a brown oil (174 mg). The crude product was purified by reverse phase preparative HPLC (RP-HPLC acidic 1: 15 to 85% B in 20 min). The product containing fractions were combined, basified with aq sat NaHCO₃, ACN was removed under reduced pressure and the aqueous residue was extracted with DCM (4×50 mL). The combined organic layers were washed with water, dried through a phase separator and concentrated under reduced pressure to give the title compound as a white solid (59.4 mg, 100% pure, yield: 48%).

The sodium salt was prepared analogous to the general procedure.

LC-MS: Rt=1.01 min; MS m/z [M+H]⁺ 717.2/719.2, m/z [M−H]⁻ 715.3/717.3; UPLC-MS 1

### Example 13: (7R,9R)—N-(2-chloro-4-(trifluoromethyl)phenyl)-2-(3,6-dihydro-2H-pyran-4-yl)-6-(4-(5-hydroxypyrimidine-4-carbonyl)piperazin-1-yl)-7-methyl-5-oxo-5,7,8,9-tetrahydropyrrolo[1,2-c][1,2,4]triazolo[1,5-a]pyrimidine-9-carboxamide

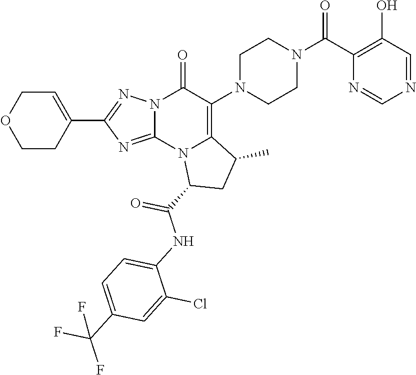 [SMILES: C[C@@H]1C[C@H](C(=O)Nc2ccc(C(F)(F)F)cc2Cl)n2c1c(N1CCN(C(=O)c3ncncc3O)CC1)c(=O)n1nc(C3=CCOCC3)nc21]

### Step 1: (7R,9R)-6-(4-(5-(benzyloxy)pyrimidine-4-carbonyl)piperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide

(7R,9R)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide (Intermediate H4) (113 mg, 196 μmol) and 5-(benzyloxy)pyrimidine-4-carboxylic acid (47.3 mg, 205 μmol) were mixed in DMF (2.7 mL) at RT under argon. HATU (92.0 mg, 235 μmol) was added, followed by DIPEA (171 μL, 978 μmol). The RM was stirred at RT for 1.75 hours. The RM was quenched with water (5 mL) and extracted with EtOAc (3×60 mL). The combined organic layers were washed with water (2×25 mL) and brine (2×20 mL), dried through a phase separator with Na₂SO₄ and concentrated under reduced pressure to give a bright brown solid (182 mg). The crude product was purified by reverse phase preparative HPLC (RP-HPLC acidic 1: 20 to 80% B in 20 min). The product containing fractions were combined and lyophilized to give the title compound as a white solid (64.0 mg, 94% pure, yield: 39%).

LC-MS: Rt=1.13 min; MS m/z [M+H]⁺ 790.5/792.5, m/z [M−H]⁻ 788.4/790.4; UPLC-MS 1

### Step 2: (7R,9R)—N-(2-chloro-4-(trifluoromethyl)phenyl)-2-(3,6-dihydro-2H-pyran-4-yl)-6-(4-(5-hydroxypyrimidine-4-carbonyl)piperazin-1-yl)-7-methyl-5-oxo-5,7,8,9-tetrahydropyrrolo[1,2-c][1,2,4]triazolo[1,5-a]pyrimidine-9-carboxamide

(7R,9R)-6-(4-(5-(benzyloxy)pyrimidine-4-carbonyl)piperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide (64.4 mg, 81.0 μmol) was dissolved in TFA (1.90 mL, 24.5 mmol) and the RM was stirred at 50° C. for 70 hours. The RM was concentrated under reduced pressure, redissolved in DCM and concentrated again, then dried under HV to give a bright brown oil (61.5 mg). The crude product was adsorbed onto Isolute and purified by column chromatography (RediSep Column: Silica 12 g gold, eluent DCM:DCM/MeOH (8/2) 100:0 to 20:80), then further purified by SFC (SFC 1). The product containing fractions were combined and concentrated under reduced pressure to give the title compound as a beige solid (28.4 mg, 100% pure, yield: 50%).

The sodium salt was prepared analogous to the general procedure.

LC-MS: Rt=0.97 min; MS m/z [M+H]⁺ 700.4/702.3, m/z [M−H]⁻ 698.3/700.3; UPLC-MS 1

LC-MS: Rt=4.91 min; MS m/z [M+H]⁺ 700.3/702.2, m/z [M−H]⁻ 698.2/700.2; UPLC-MS 2

### Example 14A: (7R,9R)—N-(2-chloro-4-(trifluoromethyl)phenyl)-2-(3,6-dihydro-2H-pyran-4-yl)-6-(4-(5-hydroxy-6-methylpyrimidine-4-carbonyl)piperazin-1-yl)-7-methyl-5-oxo-5,7,8,9-tetrahydropyrrolo[1,2-c][1,2,4]triazolo[1,5-a]pyrimidine-9-carboxamide

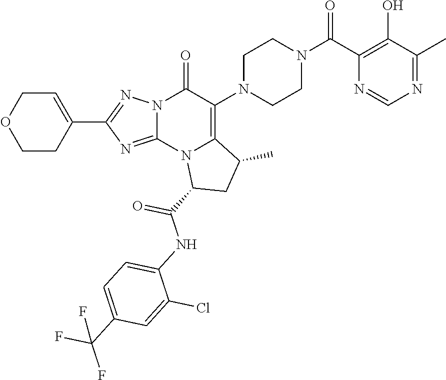 [SMILES: Cc1ncnc(C(=O)N2CCN(c3c4n(c5nc(C6=CCOCC6)nn5c3=O)[C@@H](C(=O)Nc3ccc(C(F)(F)F)cc3Cl)C[C@H]4C)CC2)c1O]

### Step 1: (7R,9R)-6-(4-(5-(benzyloxy)-6-methylpyrimidine-4-carbonyl)piperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide

To a stirred solution of (7R,9R)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide (Intermediate H4) (155 mg, 268 μmol), 5-(benzyloxy)-6-methylpyrimidine-4-carboxylic acid (Intermediate U) (65.5 mg, 268 μmol) and HATU (122 mg, 322 μmol) in DMF (2 mL) was added DIPEA (234 μL, 1.34 mmol) at RT and the RM was stirred at RT for 10 minutes. The RM was diluted with EtOAc and water, extracted twice with EtOAc and the combined organic layers were dried over Na₂SO₄ and concentrated under reduced pressure. The crude product was purified by column chromatography (RediSep Column: Silica 24 g, eluent DCM:DCM/MeOH (8/2) 100:0 to 50:50). The product containing fractions were combined, concentrated under reduced pressure and dried under HV to give the title compound (211 mg, 91% pure, yield: 89%).

LC-MS: Rt=1.20 min; MS m/z [M+H]⁺ 804.5/806.5, m/z [M−H]⁻ 802.5/804.5; UPLC-MS 1

LC-MS: Rt=5.98 min; MS m/z [M+H]⁺ 804.5/806.5, m/z [M−H]⁻ 802.5/804.5; UPLC-MS 2

### Step 2: (7R,9R)—N-(2-chloro-4-(trifluoromethyl)phenyl)-2-(3,6-dihydro-2H-pyran-4-yl)-6-(4-(5-hydroxy-6-methylpyrimidine-4-carbonyl)piperazin-1-yl)-7-methyl-5-oxo-5,7,8,9-tetrahydropyrrolo[1,2-c][1,2,4]triazolo[1,5-a]pyrimidine-9-carboxamide

(7R,9R)-6-(4-(5-(benzyloxy)-6-methylpyrimidine-4-carbonyl)piperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide (211 mg, 239 μmol) in TFA (2 mL) was stirred at 50° C. for 14 hours. The RM was concentrated under reduced pressure. The RM was diluted with DCM and aq NaHCO₃, extracted twice with DCM and the combined organic layers were washed with water and brine, dried over Na₂SO₄ and concentrated under reduced pressure. The crude product was purified by column chromatography (RediSep Column: Silica 24 g, eluent DCM:DCM/MeOH (8/2) 100:0 to 50:50) to afford a white solid (97.0 mg), which was further purified by reverse phase preparative HPLC (RP-HPLC acidic 1: 5 to 95% B in 20 min). The product containing fractions were combined, basified with aq sat NaHCO₃ and concentrated under reduced pressure to give the title compound as a white solid (107 mg, 100% pure, yield: 63%).

The sodium salt was prepared analogous to the general procedure.

LC-MS: Rt=1.06 min; MS m/z [M+H]⁺ 714.3/716.3, m/z [M−H]⁻ 712.3/714.3; UPLC-MS 1

LC-MS: Rt=5.23 min; MS m/z [M+H]⁺ 714.4/716.4, m/z [M−H]⁻ 712.4/714.4; UPLC-MS 2

1H NMR (400 MHz, DMSO-d6) δ 10.54-10.40 (m, 2H), 8.56 (s, 1H), 8.03-7.86 (m, 2H), 7.74 (dd, J=8.8, 2.1 Hz, 1H), 6.89-6.73 (m, 1H), 5.53 (dd, J=10.4, 2.3 Hz, 1H), 4.27-4.21 (m, 2H), 3.86-3.66 (m, 3H), 3.62-3.07 (m, 8H), 3.03-2.96 (m, 2H), 2.53-2.47 (m, 2H) 2.44 (s, 3H), 2.16-2.08 (m, 1H), 1.41 (d, J=7.3 Hz, 3H).

The stereochemistry of example 14A was determined by single-crystal X-ray diffraction and WRN co-crystal X-ray diffraction. By extension, Intermediate H4 was also assigned as (7R,9R).

### Example 14B: (7S,9S)—N-(2-chloro-4-(trifluoromethyl)phenyl)-2-(3,6-dihydro-2H-pyran-4-yl)-6-(4-(5-hydroxy-6-methylpyrimidine-4-carbonyl)piperazin-1-yl)-7-methyl-5-oxo-5,7,8,9-tetrahydropyrrolo[1,2-c][1,2,4]triazolo[1,5-a]pyrimidine-9-carboxamide

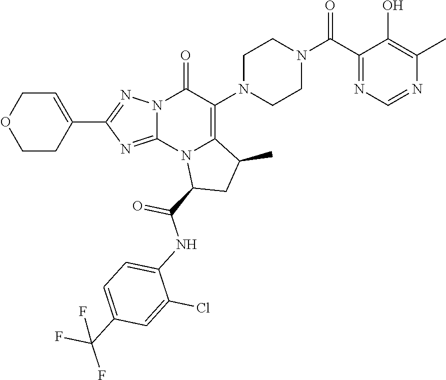 [SMILES: Cc1ncnc(C(=O)N2CCN(c3c4n(c5nc(C6=CCOCC6)nn5c3=O)[C@H](C(=O)Nc3ccc(C(F)(F)F)cc3Cl)C[C@@H]4C)CC2)c1O]

### Step 1: (7S,9S)-6-(4-(5-(benzyloxy)-6-methylpyrimidine-4-carbonyl)piperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide

To a stirred solution of (7S,9S)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide (Intermediate H3) (160 mg, 277 μmol), 5-(benzyloxy)-6-methylpyrimidine-4-carboxylic acid (Intermediate U) (67.6 mg, 277 μmol) and HATU (126 mg, 332 μmol) in DMF (2 mL) was added DIPEA (242 μL, 1.38 mmol) at RT and the RM was stirred at RT for 10 minutes. The RM was diluted with EtOAc and water, extracted twice with EtOAc and the combined organic layers were dried over Na₂SO₄ and concentrated under reduced pressure. The crude product was purified by column chromatography (RediSep Column: Silica 24 g, eluent DCM:DCM/MeOH (8/2) 100:0 to 50:50). The product containing fractions were combined, concentrated under reduced pressure and dried under HV to give the title compound as a white foam (135 mg, 100% pure, yield: 61%).

LC-MS: Rt=1.21 min; MS m/z [M+H]⁺ 804.5/806.5, m/z [M−H]⁻ 802.5/804.5; UPLC-MS 1

LC-MS: Rt=5.92 min; MS m/z [M+H]⁺ 804.5/806.5, m/z [M−H]⁻ 802.5/804.6; UPLC-MS 2

### Step 2: (7S,9S)—N-(2-chloro-4-(trifluoromethyl)phenyl)-2-(3,6-dihydro-2H-pyran-4-yl)-6-(4-(5-hydroxy-6-methylpyrimidine-4-carbonyl)piperazin-1-yl)-7-methyl-5-oxo-5,7,8,9-tetrahydropyrrolo[1,2-c][1,2,4]triazolo[1,5-a]pyrimidine-9-carboxamide

(7S,9S)-6-(4-(5-(benzyloxy)-6-methylpyrimidine-4-carbonyl)piperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide (130 mg, 162 μmol) in TFA (2 mL) was stirred at 50° C. for 4 hours. The RM was concentrated under reduced pressure. The RM was diluted with DCM and aq NaHCO₃, extracted twice with DCM and the combined organic layers were washed with water and brine, dried over Na₂SO₄ and concentrated under reduced pressure. The crude product was purified by column chromatography (RediSep Column: Silica 12 g, eluent DCM:DCM/MeOH (8/2) 100:0 to 50:50). The product containing fractions were combined and concentrated under reduced pressure to give the title compound as a white solid (97.0 mg, 100% pure, yield: 84%).

The sodium salt was prepared analogous to the general procedure.

LC-MS: Rt=1.06 min; MS m/z [M+H]⁺ 714.3/716.3, m/z [M−H]⁻ 712.3/714.3; UPLC-MS 1

LC-MS: Rt=5.17 min; MS m/z [M+H]⁺ 714.4/716.4, m/z [M−H]⁻ 712.4/714.4; UPLC-MS 2

### Example 14C: (7S,9R)—N-(2-chloro-4-(trifluoromethyl)phenyl)-2-(3,6-dihydro-2H-pyran-4-yl)-6-(4-(5-hydroxy-6-methylpyrimidine-4-carbonyl)piperazin-1-yl)-7-methyl-5-oxo-5,7,8,9-tetrahydropyrrolo[1,2-c][1,2,4]triazolo[1,5-a]pyrimidine-9-carboxamide

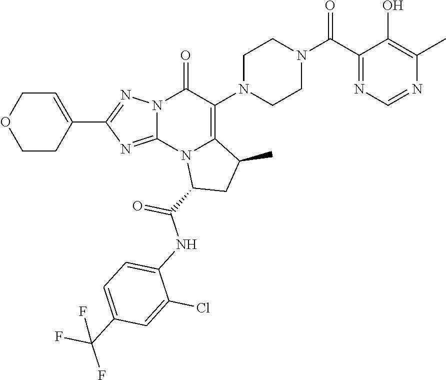 [SMILES: Cc1ncnc(C(=O)N2CCN(c3c4n(c5nc(C6=CCOCC6)nn5c3=O)[C@@H](C(=O)Nc3ccc(C(F)(F)F)cc3Cl)C[C@@H]4C)CC2)c1O]

### Step 1: (7S,9R)-6-(4-(5-(benzyloxy)-6-methylpyrimidine-4-carbonyl)piperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide

To a stirred solution of (7S,9R)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide (Intermediate H2) (495 mg, 771 μmol), 5-(benzyloxy)-6-methylpyrimidine-4-carboxylic acid (Intermediate U) (188 mg, 771 μmol) and HATU (352 mg, 925 μmol) in DMF (6 mL) was added DIPEA (673 μL, 3.85 mmol) at RT and the RM was stirred at RT for 5 minutes. The RM was diluted with EtOAc and water, extracted twice with EtOAc and the combined organic layers were dried over Na₂SO₄ and concentrated under reduced pressure. The crude product was purified by column chromatography (RediSep Column: Silica 40 g, eluent DCM:DCM/MeOH (8/2) 100:0 to 75:25). The product containing fractions were combined and concentrated under reduced pressure to give the title compound as a white foam (618 mg, 88% pure, yield: 88%).

LC-MS: Rt=1.17 min; MS m/z [M+H]⁺ 804.3/806.3, m/z [M−H]⁻ 802.3/804.3; UPLC-MS 1

### Step 2: (7S,9R)—N-(2-chloro-4-(trifluoromethyl)phenyl)-2-(3,6-dihydro-2H-pyran-4-yl)-6-(4-(5-hydroxy-6-methylpyrimidine-4-carbonyl)piperazin-1-yl)-7-methyl-5-oxo-5,7,8,9-tetrahydropyrrolo[1,2-c][1,2,4]triazolo[1,5-a]pyrimidine-9-carboxamide

(7S,9R)-6-(4-(5-(benzyloxy)-6-methylpyrimidine-4-carbonyl)piperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide (618 mg, 676 μmol) in TFA (6.00 mL, 78.0 mmol) was stirred at RT for 72 hours. The RM was diluted with DCM and aq NaHCO₃, extracted twice with DCM and the combined organic layers were washed with brine, dried over Na₂SO₄ and concentrated under reduced pressure. The crude product was purified by column chromatography (RediSep Column: Silica 40 g, eluent DCM:DCM/MeOH (8/2) 100:0 to 50:50) to afford a yellow solid, which was purified in 2 portions by reverse phase preparative HPLC (RP-HPLC acidic 1: 15 to 80% B in 20 min), (RP-HPLC acidic 1: 15 to 80% B in 20 min). The product containing fractions were combined, basified with aq sat NaHCO₃, ACN was removed under reduced pressure and the resulting aqueous residue was extracted twice with DCM. The combined organic layers were dried over Na₂SO₄ and concentrated under reduced pressure to give the title compound as a white solid (295 mg, 99% pure, yield: 61%).

LC-MS: Rt=1.02 min; MS m/z [M+H]⁺ 714.1/716.1, m/z [M−H]⁻ 712.3/714.3; UPLC-MS 1

LC-MS: Rt=5.01 min; MS m/z [M+H]⁺ 714.1/716.1, m/z [M−H]⁻ 712.3/714.3; UPLC-MS 2

The stereochemistry of example 14C as (7S,9R) was assigned due to its higher potency then 14D based SAR and structural understanding.

### Example 14D: (7R,9S)—N-(2-chloro-4-(trifluoromethyl)phenyl)-2-(3,6-dihydro-2H-pyran-4-yl)-6-(4-(5-hydroxy-6-methylpyrimidine-4-carbonyl)piperazin-1-yl)-7-methyl-5-oxo-5,7,8,9-tetrahydropyrrolo[1,2-c][1,2,4]triazolo[1,5-a]pyrimidine-9-carboxamide

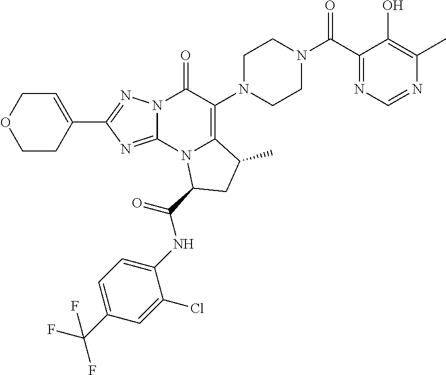 [SMILES: Cc1ncnc(C(=O)N2CCN(c3c4n(c5nc(C6=CCOCC6)nn5c3=O)[C@H](C(=O)Nc3ccc(C(F)(F)F)cc3Cl)C[C@H]4C)CC2)c1O]

### Step 1: (7R,9S)-6-(4-(5-(benzyloxy)-6-methylpyrimidine-4-carbonyl)piperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide

To a stirred solution of (7R,9S)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide (Intermediate H1) (466 mg, 806 μmol), 5-(benzyloxy)-6-methylpyrimidine-4-carboxylic acid (Intermediate U) (197 mg, 806 μmol) and HATU (368 mg, 967 μmol) in DMF (5 mL) was added DIPEA (704 μL, 4.03 μmol) at RT and the RM was stirred at RT for 5 minutes. The RM was diluted with EtOAc and water, extracted twice with EtOAc and the combined organic layers were dried over Na₂SO₄ and concentrated under reduced pressure. The crude product was purified by column chromatography (RediSep Column: Silica 40 g, eluent DCM:DCM/MeOH (8/2) 100:0 to 50:50). The product containing fractions were combined and concentrated under reduced pressure to give the title compound as a white foam (618 mg, 90% pure, yield: 86%).

LC-MS: Rt=1.17 min; MS m/z [M+H]⁺ 804.4/806.4, m/z [M−H]⁻ 802.3/804.3; UPLC-MS 1

### Step 2: (7R,9S)—N-(2-chloro-4-(trifluoromethyl)phenyl)-2-(3,6-dihydro-2H-pyran-4-yl)-6-(4-(5-hydroxy-6-methylpyrimidine-4-carbonyl)piperazin-1-yl)-7-methyl-5-oxo-5,7,8,9-tetrahydropyrrolo[1,2-c][1,2,4]triazolo[1,5-a]pyrimidine-9-carboxamide

(7R,9S)-6-(4-(5-(benzyloxy)-6-methylpyrimidine-4-carbonyl)piperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide (618 mg, 692 μmol) in TFA (6.00 mL, 78.0 mmol) was stirred at 50° C. for 14 hours. The RM was diluted with DCM and aq NaHCO₃, extracted twice with DCM and the combined organic layers were washed with brine, dried over Na₂SO₄ and concentrated under reduced pressure. The crude product was purified by column chromatography (RediSep Column: Silica 40 g, eluent DCM:DCM/MeOH (8/2) 100:0 to 50:50) to afford a yellow solid, which was purified in 2 portions by reverse phase preparative HPLC (RP-HPLC acidic 1: 15 to 80% B in 20 min). The product containing fractions were combined, basified with aq sat NaHCO₃, ACN was removed under reduced pressure and the resulting aqueous residue was extracted twice with DCM. The combined organic layers were dried over Na₂SO₄ and concentrated under reduced pressure to give the title compound as a white solid (283 mg, 96% pure, yield: 55%).

LC-MS: Rt=1.02 min; MS m/z [M+H]⁺ 714.3/716.3, m/z [M−H]⁻ 712.3/714.3; UPLC-MS 1

LC-MS: Rt=5.05 min; MS m/z [M+H]⁺ 714.2/716.2, m/z [M−H]⁻ 712.2/714.2; UPLC-MS 2

### Example 15A: (7R,9R)-2-(3,6-dihydro-2H-pyran-4-yl)-6-(4-(3-hydroxypicolinoyl)piperazin-1-yl)-7-methyl-N-(2-methyl-4-(trifluoromethyl)phenyl)-5-oxo-5,7,8,9-tetrahydropyrrolo[1,2-c][1,2,4]triazolo[1,5-a]pyrimidine-9-carboxamide

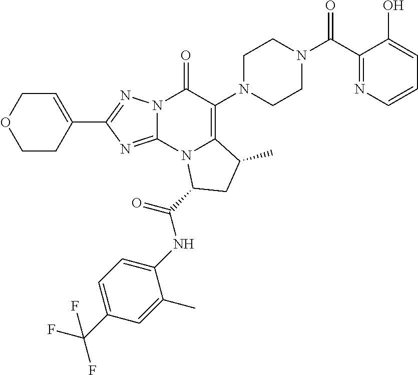 [SMILES: Cc1cc(C(F)(F)F)ccc1NC(=O)[C@H]1C[C@@H](C)c2c(N3CCN(C(=O)c4ncccc4O)CC3)c(=O)n3nc(C4=CCOCC4)nc3n21]

To a colourless solution of (7R,9R)-2-(3,6-dihydro-2H-pyran-4-yl)-7-methyl-N-(2-methyl-4-(trifluoromethyl)phenyl)-5-oxo-6-(piperazin-1-yl)-5,7,8,9-tetrahydropyrrolo[1,2-c][1,2,4]triazolo[1,5-a]pyrimidine-9-carboxamide (Intermediate K4) (25.0 mg, 40.0 μmol) in DCM (2 mL), was added DIPEA (35.0 μL, 198 μmol), followed by 3-hydroxypicolinoyl chloride (Intermediate X) (9.37 mg, 59.0 μmol) at RT. The RM was stirred at RT for 3.5 hours. The RM was extracted with water (10 mL) and TBME (10 mL). The organic layer was washed with water (15 mL) and brine (15 mL). The aqueous layer was washed with TBME (2×15 mL). The combined organic layers were dried over anhydrous Na₂SO₄, filtered and concentrated under reduced pressure at 45° C. to give a brown solid (49.5 mg). The crude product was purified by reverse phase preparative HPLC (RP-HPLC acidic 1: 5 to 100% B in 20 min). The product containing fractions were combined and the ACN was removed under reduced pressure. The aqueous residue was basified with aq sat NaHCO₃ and extracted with DCM (3×15 mL). The combined organic layers were dried over anhydrous Na₂SO₄, filtered and concentrated under reduced pressure to give the title compound as a white solid (17.0 mg, 98% pure, yield: 62%).

The sodium salt was prepared analogous to the general procedure.

LC-MS: Rt=1.00 min; MS m/z [M+H]⁺ 679.5, m/z [M−H]⁻ 677.5; UPLC-MS 1

LC-MS: Rt=4.78 min; MS m/z [M+H]⁺ 679.6, m/z [M−H]⁻ 677.5; UPLC-MS 2

The stereochemistry of example 15A was determined by single-crystal X-ray diffraction and WRN co-crystal X-ray diffraction. By extension, Intermediate K4 was also assigned as (7R,9R).

### Example 15B: (7S,9S)-2-(3,6-dihydro-2H-pyran-4-yl)-6-(4-(3-hydroxypicolinoyl)piperazin-1-yl)-7-methyl-N-(2-methyl-4-(trifluoromethyl)phenyl)-5-oxo-5,7,8,9-tetrahydropyrrolo[1,2-c][1,2,4]triazolo[1,5-a]pyrimidine-9-carboxamide

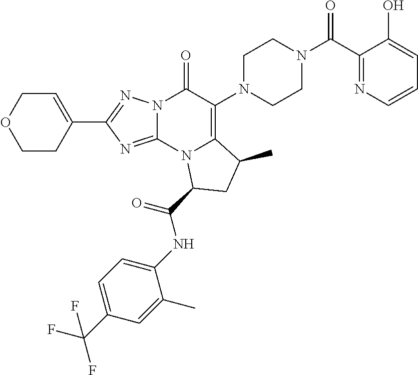 [SMILES: Cc1cc(C(F)(F)F)ccc1NC(=O)[C@@H]1C[C@H](C)c2c(N3CCN(C(=O)c4ncccc4O)CC3)c(=O)n3nc(C4=CCOCC4)nc3n21]

To a colourless solution of (7S,9S)-2-(3,6-dihydro-2H-pyran-4-yl)-7-methyl-N-(2-methyl-4-(trifluoromethyl)phenyl)-5-oxo-6-(piperazin-1-yl)-5,7,8,9-tetrahydropyrrolo[1,2-c][1,2,4]triazolo[1,5-a]pyrimidine-9-carboxamide (Intermediate K3) (23.0 mg, 36.0 μmol) in DCM (2 mL), was added DIPEA (32.0 μL, 182 μmol), followed by 3-hydroxypicolinoyl chloride (Intermediate X) (8.62 mg, 55.0 μmol) at RT. The RM was stirred at RT for 3.5 hours. The RM was extracted with water (10 mL) and TBME (10 mL). The organic layer was washed with water (15 mL) and brine (15 mL). The aqueous layer was washed with TBME (2×15 mL). The combined organic layers were dried over anhydrous Na₂SO₄, filtered and concentrated under reduced pressure at 45° C. to give a pale brown solid (30.1 mg). The crude product was purified by reverse phase preparative HPLC (RP-HPLC acidic 1: 5 to 100% B in 20 min). The product containing fractions were combined and the ACN was removed under reduced pressure. The aqueous residue was basified with aq sat NaHCO₃ and extracted with DCM (3×15 mL). The combined organic layers were dried over anhydrous Na₂SO₄, filtered and concentrated under reduced pressure to give the title compound as a white solid (9.50 mg, 87% pure, yield: 33%).

LC-MS: Rt=1.00 min; MS m/z [M+H]⁺ 679.6, m/z [M−H]⁻ 677.5; UPLC-MS 1

LC-MS: Rt=4.79 min; MS m/z [M+H]⁺ 679.6, m/z [M−H]⁻ 677.5; UPLC-MS 2

### Example 15C: (7S,9R)-2-(3,6-dihydro-2H-pyran-4-yl)-6-(4-(3-hydroxypicolinoyl)piperazin-1-yl)-7-methyl-N-(2-methyl-4-(trifluoromethyl)phenyl)-5-oxo-5,7,8,9-tetrahydropyrrolo[1,2-c][1,2,4]triazolo[1,5-a]pyrimidine-9-carboxamide

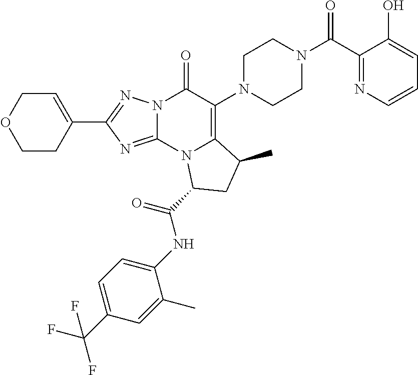 [SMILES: Cc1cc(C(F)(F)F)ccc1NC(=O)[C@H]1C[C@H](C)c2c(N3CCN(C(=O)c4ncccc4O)CC3)c(=O)n3nc(C4=CCOCC4)nc3n21]

To a colourless solution of (7S,9R)-2-(3,6-dihydro-2H-pyran-4-yl)-7-methyl-N-(2-methyl-4-(trifluoromethyl)phenyl)-5-oxo-6-(piperazin-1-yl)-5,7,8,9-tetrahydropyrrolo[1,2-c][1,2,4]triazolo[1,5-a]pyrimidine-9-carboxamide (Intermediate K1) (24.0 mg, 40.0 μmol) in DCM (50 mL), was added DIPEA (35.0 μL, 202 μmol), followed by 3-hydroxypicolinoyl chloride (Intermediate X) (9.55 mg, 61.0 μmol) at RT. The RM was stirred at RT for 3.5 hours. The RM was extracted with water (10 mL) and TBME (10 mL). The organic layer was washed with water (15 mL) and brine (15 mL). The aqueous layer was washed with TBME (2×15 mL). The combined organic layers were dried over anhydrous Na₂SO₄, filtered and concentrated under reduced pressure at 45° C. to give a brown solid (33.0 mg). The crude product was purified by column chromatography (RediSep Column: Silica 24 g, eluent DCM:DCM/MeOH (9/1) 100:0 to 20:80). The product containing fractions were combined and concentrated under reduced pressure to give the title compound as a pale brown solid (14.7 mg, 95% pure, yield: 51%).

LC-MS: Rt=0.97 min; MS m/z [M+H]⁺ 679.6, m/z [M−H]⁻ 677.5; UPLC-MS 1

LC-MS: Rt=6.56 min; MS m/z [M+H]⁺ 679.6, m/z [M−H]⁻ 677.6; UPLC-MS 2

The stereochemistry of example 15C as (7S,9R) was assigned due to its potency and an understanding of structure-activity relationships.

### Example 15D: (7R,9S)-2-(3,6-dihydro-2H-pyran-4-yl)-6-(4-(3-hydroxypicolinoyl)piperazin-1-yl)-7-methyl-N-(2-methyl-4-(trifluoromethyl)phenyl)-5-oxo-5,7,8,9-tetrahydropyrrolo[1,2-c][1,2,4]triazolo[1,5-a]pyrimidine-9-carboxamide

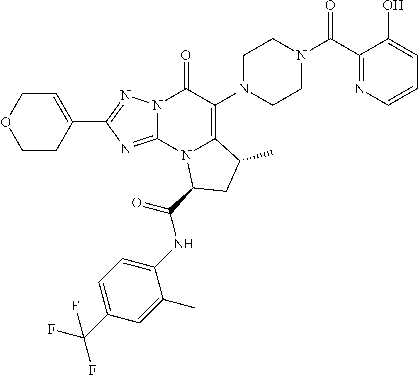 [SMILES: Cc1cc(C(F)(F)F)ccc1NC(=O)[C@@H]1C[C@@H](C)c2c(N3CCN(C(=O)c4ncccc4O)CC3)c(=O)n3nc(C4=CCOCC4)nc3n21]

To a colourless solution of (7R,9S)-2-(3,6-dihydro-2H-pyran-4-yl)-7-methyl-N-(2-methyl-4-(trifluoromethyl)phenyl)-5-oxo-6-(piperazin-1-yl)-5,7,8,9-tetrahydropyrrolo[1,2-c][1,2,4]triazolo[1,5-a]pyrimidine-9-carboxamide (Intermediate K2) (24.0 mg, 40.0 μmol) in DCM (5 mL), was added DIPEA (35.0 mL, 202 μmol), followed by 3-hydroxypicolinoyl chloride (Intermediate X) (9.55 mg, 61.0 μmol) at RT. The RM was stirred at RT for 3.5 hours. The RM was extracted with water (10 mL) and TBME (10 mL). The organic layer was washed with water (15 mL) and brine (15 mL). The aqueous layer was washed with TBME (2×15 mL). The combined organic layers were dried over anhydrous Na₂SO₄, filtered and concentrated under reduced pressure at 45° C. to give a brown solid (36.0 mg). The crude product was purified by column chromatography (RediSep Column: Silica 24 g, eluent DCM:DCM/MeOH (9/1) 100:0 to 20:80). The product containing fractions were combined and concentrated under reduced pressure to give the title compound as a pale brown solid (13.5 mg, 96% pure, yield: 47%).

LC-MS: Rt=0.97 min; MS m/z [M+H]⁺ 679.6, m/z [M−H]⁻ 677.5; UPLC-MS 1

LC-MS: Rt=4.65 min; MS m/z [M+H]⁺ 679.6, m/z [M−H]⁻ 677.5; UPLC-MS 2

### Example 16: (7R,9R)-2-(3,6-dihydro-2H-pyran-4-yl)-6-(4-(4-fluoro-3-hydroxypicolinoyl)piperazin-1-yl)-7-methyl-N-(2-methyl-4-(trifluoromethyl)phenyl)-5-oxo-5,7,8,9-tetrahydropyrrolo[1,2-c][1,2,4]triazolo[1,5-a]pyrimidine-9-carboxamide

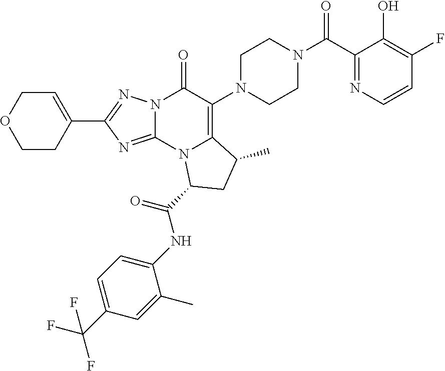 [SMILES: Cc1cc(C(F)(F)F)ccc1NC(=O)[C@H]1C[C@@H](C)c2c(N3CCN(C(=O)c4nccc(F)c4O)CC3)c(=O)n3nc(C4=CCOCC4)nc3n21]

### Step 1: (7R,9R)-6-(4-(3-(benzyloxy)-4-fluoropicolinoyl)piperazin-1-yl)-2-(3,6-dihydro-2H-pyran-4-yl)-7-methyl-N-(2-methyl-4-(trifluoromethyl)phenyl)-5-oxo-5,7,8,9-tetrahydropyrrolo[1,2-c][1,2,4]triazolo[1,5-a]pyrimidine-9-carboxamide

To a stirred solution of (7R,9R)-2-(3,6-dihydro-2H-pyran-4-yl)-7-methyl-N-(2-methyl-4-(trifluoromethyl)phenyl)-5-oxo-6-(piperazin-1-yl)-5,7,8,9-tetrahydropyrrolo[1,2-c][1,2,4]triazolo[1,5-a]pyrimidine-9-carboxamide (Intermediate K4) (175 mg, 311 μmol), 3-(benzyloxy)-4-fluoropicolinic acid (Intermediate W) (81.0 mg, 326 μmol) and HATU (142 mg, 373 μmol) in DMF (2 mL) was added DIPEA (313 μL, 1.79 mmol) at RT and the RM was stirred at RT for 10 minutes. The RM was diluted with EtOAc and water, extracted twice with EtOAc and the combined organic layers were dried over Na₂SO₄ and concentrated under reduced pressure. The crude product was purified by column chromatography (RediSep Column: Silica 24 g, eluent DCM:DCM/MeOH (8/2) 100:0 to 60:40). The product containing fractions were combined and concentrated under reduced pressure to give the title compound as a white foam (220 mg, 100% pure, yield: 90%).

LC-MS: Rt=1.20 min; MS m/z [M+H]⁺ 787.5, m/z [M−H]⁻ 785.6; UPLC-MS 1

LC-MS: Rt=5.91 min; MS m/z [M+H]⁺ 787.5, m/z [M−H]⁻ 785.5; UPLC-MS 2

### Step 2: (7R,9R)-2-(3,6-dihydro-2H-pyran-4-yl)-6-(4-(4-fluoro-3-hydroxypicolinoyl)piperazin-1-yl)-7-methyl-N-(2-methyl-4-(trifluoromethyl)phenyl)-5-oxo-5,7,8,9-tetrahydropyrrolo[1,2-c][1,2,4]triazolo[1,5-a]pyrimidine-9-carboxamide

(7R,9R)-6-(4-(3-(benzyloxy)-4-fluoropicolinoyl)piperazin-1-yl)-2-(3,6-dihydro-2H-pyran-4-yl)-7-methyl-N-(2-methyl-4-(trifluoromethyl)phenyl)-5-oxo-5,7,8,9-tetrahydropyrrolo[1,2-c][1,2,4]triazolo[1,5-a]pyrimidine-9-carboxamide (220 mg, 280 μmol) was dissolved in TFA (3.00 mL, 39.0 mmol) and the RM was stirred at 50° C. overnight. The RM was cooled to RT and poured into aq NaHCO₃. Water (40 mL) was added and the aqueous layer was extracted with DCM (2×100 mL). The combined organic layers were dried over MgSO₄, filtered and concentrated under reduced pressure. The crude product was purified by column chromatography (RediSep Column: Silica 24 g, eluent DCM:DCM/MeOH (8/2) 100:0 to 60:40). The product containing fractions were combined and concentrated under reduced pressure. The residue was redissolved in EtOH, heated at 60° C. overnight and cooled down. The suspension was filtered and dried under HV to give the title compound as a white solid (81.8 mg, 98% pure, yield: 41%).

The sodium salt was prepared analogous to the general procedure.

LC-MS: Rt=0.99 min; MS m/z [M+H]⁺ 697.3, m/z [M−H]⁻ 695.5; UPLC-MS 1

### Example 17: (7R,9R)-2-(3,6-dihydro-2H-pyran-4-yl)-6-(4-(5-hydroxy-6-methylpyrimidine-4-carbonyl)piperazin-1-yl)-7-methyl-N-(2-methyl-4-(trifluoromethyl)phenyl)-5-oxo-5,7,8,9-tetrahydropyrrolo[1,2-c][1,2,4]triazolo[1,5-a]pyrimidine-9-carboxamide

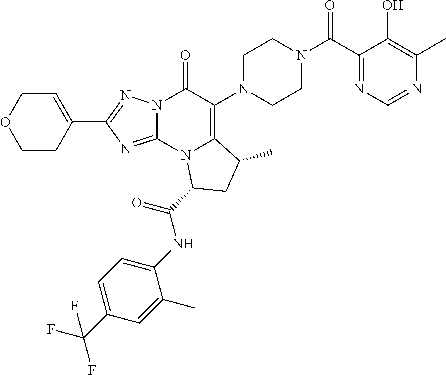 [SMILES: Cc1cc(C(F)(F)F)ccc1NC(=O)[C@H]1C[C@@H](C)c2c(N3CCN(C(=O)c4ncnc(C)c4O)CC3)c(=O)n3nc(C4=CCOCC4)nc3n21]

### Step 1: (7R,9R)-6-(4-(5-(benzyloxy)-6-methylpyrimidine-4-carbonyl)piperazin-1-yl)-2-(3,6-dihydro-2H-pyran-4-yl)-7-methyl-N-(2-methyl-4-(trifluoromethyl)phenyl)-5-oxo-5,7,8,9-tetrahydropyrrolo[1,2-c][1,2,4]triazolo[1,5-a]pyrimidine-9-carboxamide

To a stirred solution of (7R,9R)-2-(3,6-dihydro-2H-pyran-4-yl)-7-methyl-N-(2-methyl-4-(trifluoromethyl)phenyl)-5-oxo-6-(piperazin-1-yl)-5,7,8,9-tetrahydropyrrolo[1,2-c][1,2,4]triazolo[1,5-a]pyrimidine-9-carboxamide (Intermediate K4) (200 mg, 355 μmol), 5-(benzyloxy)-6-methylpyrimidine-4-carboxylic acid (Intermediate U) (91.0 mg, 373 μmol) and HATU (162 mg, 426 μmol) in DMF (2 mL) was added DIPEA (313 μL, 1.79 mmol) at RT and the RM was stirred at RT for 10 minutes. The RM was diluted with EtOAc and water, extracted twice with EtOAc and the combined organic layers were dried over Na₂SO₄ and concentrated under reduced pressure. The crude product was purified by column chromatography (RediSep Column: Silica 24 g, eluent DCM:DCM/MeOH (8/2) 100:0 to 60:40). The product containing fractions were combined and concentrated under reduced pressure to give the title compound as a white foam (225 mg, 99% pure, yield: 80%).

LC-MS: Rt=1.16 min; MS m/z [M+H]⁺ 784.3, m/z [M−H]⁻ 782.4; UPLC-MS 1

LC-MS: Rt=5.68 min; MS m/z [M+H]⁺ 784.4, m/z [M−H]⁻ 782.6; UPLC-MS 2

### Step 2: (7R,9R)-2-(3,6-dihydro-2H-pyran-4-yl)-6-(4-(5-hydroxy-6-methylpyrimidine-4-carbonyl)piperazin-1-yl)-7-methyl-N-(2-methyl-4-(trifluoromethyl)phenyl)-5-oxo-5,7,8,9-tetrahydropyrrolo[1,2-c][1,2,4]triazolo[1,5-a]pyrimidine-9-carboxamide

(7R,9R)-6-(4-(5-(benzyloxy)-6-methylpyrimidine-4-carbonyl)piperazin-1-yl)-2-(3,6-dihydro-2H-pyran-4-yl)-7-methyl-N-(2-methyl-4-(trifluoromethyl)phenyl)-5-oxo-5,7,8,9-tetrahydropyrrolo[1,2-c][1,2,4]triazolo[1,5-a]pyrimidine-9-carboxamide (220 mg, 278 μmol) in TFA (3.00 mL, 38.9 mmol) was stirred at 50° C. for 4 hours. The RM was diluted with DCM and aq NaHCO₃, extracted twice with DCM and the combined organic layers were washed with brine, dried over Na₂SO₄ and concentrated under reduced pressure. The crude product was purified by column chromatography (RediSep Column: Silica 24 g, eluent DCM:DCM/MeOH (8/2) 100:0 to 50:50). The product containing fractions were combined and concentrated under reduced to give the title compound as a white solid (154 mg, 98% pure, yield: 78%).

The sodium salt was prepared analogous to the general procedure.

LC-MS: Rt=1.01 min; MS m/z [M+H]⁺ 694.3, m/z [M−H]⁻ 692.5; UPLC-MS 1

LC-MS: Rt=4.94 min; MS m/z [M+H]⁺ 694.3, m/z [M−H]⁻ 692.5; UPLC-MS 2

### Example 18A: (7R,9R)—N-(2-chloro-4-(trifluoromethyl)phenyl)-2-(3,6-dihydro-2H-pyran-4-yl)-6-((R)-4-(5-hydroxy-6-methylpyrimidine-4-carbonyl)-3-methylpiperazin-1-yl)-7-methyl-5-oxo-5,7,8,9-tetrahydropyrrolo[1,2-c][1,2,4]triazolo[1,5-a]pyrimidine-9-carboxamide

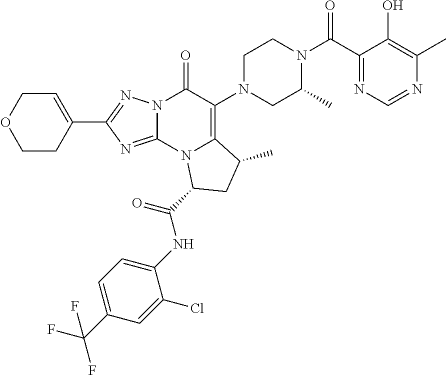 [SMILES: Cc1ncnc(C(=O)N2CCN(c3c4n(c5nc(C6=CCOCC6)nn5c3=O)[C@@H](C(=O)Nc3ccc(C(F)(F)F)cc3Cl)C[C@H]4C)C[C@H]2C)c1O]

To a stirred suspension of 5-hydroxy-6-methylpyrimidine-4-carboxylic acid (312 mg, 2.03 mmol) in DCM (10 mL) was added at RT 1-chloro-N,N,2-trimethylprop-1-en-1-amine (346 μL, 2.62 mmol) and the brown solution was stirred at RT for 1 hour, then (7R,9R)—N-(2-chloro-4-(trifluoromethyl)phenyl)-2-(3,6-dihydro-2H-pyran-4-yl)-7-methyl-6-((R)-3-methylpiperazin-1-yl)-5-oxo-5,7,8,9-tetrahydropyrrolo[1,2-c][1,2,4]triazolo[1,5-a]pyrimidine-9-carboxamide (Intermediate N2) (1.00 g, 1.69 mmol) was added. After 30 minutes at RT, a solution of DIPEA (883 μL, 5.07 mmol) in DCM (1 mL) was added dropwise. The RM was stirred at RT for 3 hours. The reaction was quenched with 1.5% HCl solution and the mixture was filtered through celite. The filtrate was extracted twice with DCM and the combined organic layers were treated with 2% NaOH solution. The basic phase was washed with DCM, then it was acidified with 3% HCl till pH=5-7 and was extracted twice with DCM. The organic phase was dried over Na₂SO₄ and concentrated under reduced pressure. The crude product was purified by column chromatography (RediSep Column: Silica 24 g, eluent DCM:DCM/MeOH (8/2) 100:0 to 60:40), then by reverse phase preparative HPLC (RP-HPLC acidic 1: 30 to 61% B in 18 min). The product containing fractions were combined, basified with aq sat NaHCO₃, ACN was removed under reduced pressure and the aqueous residue was extracted twice with DCM. The combined organic layers were dried over Na₂SO₄ and concentrated under reduced pressure to give the title compound as a white solid (419 mg, 99% pure, yield: 34%).

Trituration overnight in 10% water/iPrOH afforded crystalline material of Example 18A, as (7R,9R)—N-(2-chloro-4-(trifluoromethyl)phenyl)-2-(3,6-dihydro-2H-pyran-4-yl)-6-((R)-4-(5-hydroxy-6-methylpyrimidine-4-carbonyl)-3-methylpiperazin-1-yl)-7-methyl-5-oxo-5,7,8,9-tetrahydropyrrolo[1,2-c][1,2,4]triazolo[1,5-a]pyrimidine-9-carboxamide monohydrate. The sample was characterized by the XPRD diffractogram in FIG. 1. The table Ex 18A below shows the most prominent peaks (deg 2theta) of the XRPD diffractogram of FIG. 1.

TABLE 18A

| Angle 2-theta | D Value | Relative intensity % |
|---|---|---|
| 8.6 | 10.3 | 65.5 |
| 11.2 | 7.9 | 34.9 |
| 13.0 | 6.8 | 29.1 |
| 14.9 | 5.9 | 100.0 |
| 15.5 | 5.7 | 88.4 |
| 17.9 | 5.0 | 32.7 |
| 19.4 | 4.6 | 86.4 |
| 22.0 | 4.0 | 98.8 |
| 24.3 | 3.7 | 30.2 |
| 26.0 | 3.4 | 44.2 |
| 28.2 | 3.2 | 10.1 |
| 29.1 | 3.1 | 26.0 |
| 29.7 | 3.0 | 25.9 |

LC-MS: Rt=1.08 min; MS m/z [M+H]⁺ 728.6/730.5, m/z [M−H]⁻ 726.4/728.4; UPLC-MS 1

LC-MS: Rt=5.27 min; MS m/z [M+H]⁺ 728.1/730.1, m/z [M−H]⁻ 726.3/728.3; UPLC-MS 2

¹H NMR (400 MHz, DMSO) δ 10.50 (s, 1H), 10.19 (s, 1H), 8.56 (s, 1H), 8.01-7.89 (m, 2H), 7.74 (dd, J=9.0, 4.6 Hz, 1H), 6.83-6.75 (m, 1H), 5.60-5.46 (m, 1H), 4.83-4.34 (m, 1H), 4.28-4.19 (m, 2H), 3.84-3.75 (m, 2H), 3.74-3.46 (m, 4H), 3.44-2.50 (m, 6H), 2.44 (s, 3H), 2.14 (d, J=13.6 Hz, 1H), 1.50-1.31 (m, 6H).

The stereochemistry of example 18A was determined by single-crystal X-ray diffraction and WRN co-crystal X-ray diffraction. By extension, Intermediate N2 was also assigned as (7R,9R).

The sodium salt was prepared analogous to the general procedure.

### Example 18B: (7S,9S)—N-(2-chloro-4-(trifluoromethyl)phenyl)-2-(3,6-dihydro-2H-pyran-4-yl)-6-((R)-4-(5-hydroxy-6-methylpyrimidine-4-carbonyl)-3-methylpiperazin-1-yl)-7-methyl-5-oxo-5,7,8,9-tetrahydropyrrolo[1,2-c][1,2,4]triazolo[1,5-a]pyrimidine-9-carboxamide

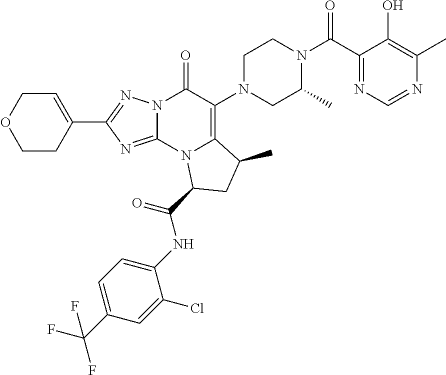 [SMILES: Cc1ncnc(C(=O)N2CCN(c3c4n(c5nc(C6=CCOCC6)nn5c3=O)[C@H](C(=O)Nc3ccc(C(F)(F)F)cc3Cl)C[C@@H]4C)C[C@H]2C)c1O]

### Step 1: (7S,9S)-6-((R)-4-(5-(benzyloxy)-6-methylpyrimidine-4-carbonyl)-3-methylpiperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide

(7S,9S)—N-(2-chloro-4-(trifluoromethyl)phenyl)-2-(3,6-dihydro-2H-pyran-4-yl)-7-methyl-6-((R)-3-methylpiperazin-1-yl)-5-oxo-5,7,8,9-tetrahydropyrrolo[1,2-c][1,2,4]triazolo[1,5-a]pyrimidine-9-carboxamide (Intermediate N1) was redissolved in DMF (2 mL) and Et₃N (48.0 μL, 347 μmol) was added, followed by 5-(benzyloxy)-6-methylpyrimidine-4-carboxylic acid (Intermediate U) (21.2 mg, 87.0 μmol). The RM was sonicated until full dissolution. HATU (36.3 mg, 95.0 μmol) was added and the RM was sonicated until full dissolution and stood at RT for 10 minutes. The RM was diluted with water (1 mL) and stirred at RT for 30 minutes. Then it was filtered and the solid was washed with water and dried on the filter pad to give the title compound as a yellow solid (50.0 mg, 93% pure, yield: 66%).

LC-MS: Rt=1.24 min; MS m/z [M+H]⁺ 818.4/820.3, m/z [M−H]⁻ 816.4/818.3; UPLC-MS 1

### Step 2: (7S,9S)—N-(2-chloro-4-(trifluoromethyl)phenyl)-2-(3,6-dihydro-2H-pyran-4-yl)-6-((R)-4-(5-hydroxy-6-methylpyrimidine-4-carbonyl)-3-methylpiperazin-1-yl)-7-methyl-5-oxo-5,7,8,9-tetrahydropyrrolo[1,2-c][1,2,4]triazolo[1,5-a]pyrimidine-9-carboxamide

(7S,9S)-6-((R)-4-(5-(benzyloxy)-6-methylpyrimidine-4-carbonyl)-3-methylpiperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide (50.0 mg, 61.0 μmol) was heated in TFA (500 μL, 6.49 mmol) at 50° C. for 2 hours. The RM was concentrated under reduced pressure. The crude product was purified by reverse phase preparative HPLC (RP-HPLC acidic 1: 20 to 65% B in 10 min). The product containing fractions were combined and basified to pH=8 with aq sat NaHCO₃. The aqueous residue was extracted with DCM (2×30 mL). The combined organic layers were dried over Na₂SO₄, filtered and concentrated under reduced pressure to give the title compound as an off-white solid (28.8 mg, 99% pure, yield: 64%).

The sodium salt was prepared analogous to the general procedure.

LC-MS: Rt=1.08 min; MS m/z [M+H]⁺ 728.3/730.3, m/z [M−H]⁻ 726.3/728.4; UPLC-MS 1

LC-MS: Rt=5.42 min; MS m/z [M+H]⁺ 728.3/730.2, m/z [M−H]⁻ 726.3/728.3; UPLC-MS 2

The stereochemistry of example 18B was determined by single-crystal X-ray diffraction. By extension, Intermediate N1 was also assigned as (7S,9S).

### Example 18C: (7R,9S)—N-(2-chloro-4-(trifluoromethyl)phenyl)-2-(3,6-dihydro-2H-pyran-4-yl)-6-((R)-4-(5-hydroxy-6-methylpyrimidine-4-carbonyl)-3-methylpiperazin-1-yl)-7-methyl-5-oxo-5,7,8,9-tetrahydropyrrolo[1,2-c][1,2,4]triazolo[1,5-a]pyrimidine-9-carboxamide

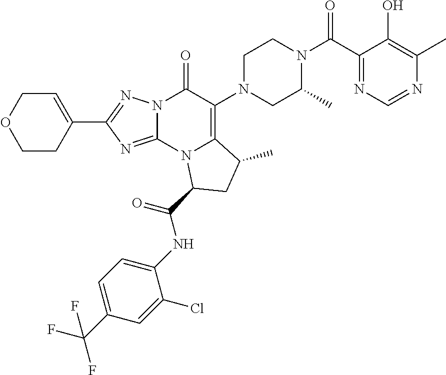 [SMILES: Cc1ncnc(C(=O)N2CCN(c3c4n(c5nc(C6=CCOCC6)nn5c3=O)[C@H](C(=O)Nc3ccc(C(F)(F)F)cc3Cl)C[C@H]4C)C[C@H]2C)c1O]

Step 1: To tert-butyl (R)-4-((7R,9S)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)-2-methylpiperazine-1-carboxylate (Intermediate M3) (418 mg, 604 μmol) was added 4N HCl/dioxane (3 ml) and the mixture stood at RT for 5 h. Evaporation in vacuo gave (7R,9S)—N-(2-chloro-4-(trifluoromethyl)phenyl)-2-(3,6-dihydro-2H-pyran-4-yl)-7-methyl-6-((R)-3-methylpiperazin-1-yl)-5-oxo-5,7,8,9-tetrahydropyrrolo[1,2-c][1,2,4]triazolo[1,5-a]pyrimidine-9-carboxamide-2-methylpropan-2-ol (1/1) HCl salt as a colourless solid that was used without further purification in the next step.

Step 2: The material from step 1 was resuspended in DMF (3 ml), 5-(benzyloxy)-6-methylpyrimidine-4-carboxylic acid (148 mg, 604 μmol) was added, followed by DIPEA (312 mg, 421 μL, 2.42 mmol) and the resulting mixture was stirred at RT for 10 mins. Then HATU (230 mg, 604 μmol) was added and the mixture stirred at RT for 1 min. The reaction was quenched with water (2 ml) and the resulting suspension was stirred at RT for 10 mins, filtered, washed with water (5 ml) and dried under vacuum to give (7R,9S)-6-((R)-4-(5-(benzyloxy)-6-methylpyrimidine-4-carbonyl)-3-methylpiperazin-1-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide as a beige solid.

Step 3: The material from step 3 was redissolved in TFA (3 ml) and heated at 45° C. overnight. The reaction mixture was evaporated in vacuo, redissolved in CH₃CN/water and directly purified by RP Prep HPLC. The fractions containing the title compound were combined and partitioned between DCM (30 ml) and sat. NaHCO₃ (aq) (10 ml). The organic layer was separated. The aqueous layer was extracted with DCM (30 ml). The combined organic layers were dried (Na₂SO₄), filtered and evaporated to give (7R,9S)—N-(2-chloro-4-(trifluoromethyl)phenyl)-2-(3,6-dihydro-2H-pyran-4-yl)-6-((R)-4-(5-hydroxy-6-methylpyrimidine-4-carbonyl)-3-methylpiperazin-1-yl)-7-methyl-5-oxo-5,7,8,9-tetrahydropyrrolo[1,2-c][1,2,4]triazolo[1,5-a]pyrimidine-9-carboxamide as a colourless solid (32 mg, 99% purity).

LC-MS: Rt=1.11 min; MS m/z [M+H]⁺ 728.4/730.4, m/z [M−H]⁻ 726.2/728.2; UPLC-MS 1

The stereochemistry of example 18C was determined by single-crystal X-ray diffraction.

### Example 18D: (7S,9R)—N-(2-chloro-4-(trifluoromethyl)phenyl)-2-(3,6-dihydro-2H-pyran-4-yl)-6-((R)-4-(5-hydroxy-6-methylpyrimidine-4-carbonyl)-3-methylpiperazin-1-yl)-7-methyl-5-oxo-5,7,8,9-tetrahydropyrrolo[1,2-c][1,2,4]triazolo[1,5-a]pyrimidine-9-carboxamide

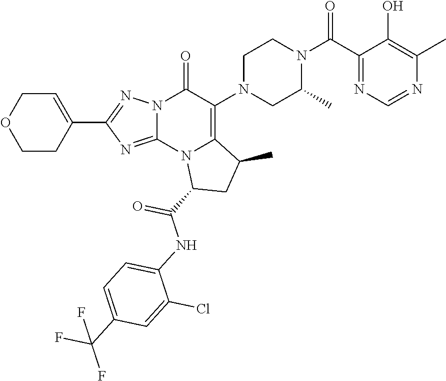 [SMILES: Cc1ncnc(C(=O)N2CCN(c3c4n(c5nc(C6=CCOCC6)nn5c3=O)[C@@H](C(=O)Nc3ccc(C(F)(F)F)cc3Cl)C[C@@H]4C)C[C@H]2C)c1O]

Step 1: To tert-butyl (R)-4-((7S,9R)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)-2-methylpiperazine-1-carboxylate (Intermediate M1) (383 mg, 553 μmol) in 4N HCl in dioxane (3 g, 2 mL, 4 molar, 8 mmol) was added MeOH (1 mL). The mixture was sonicated until full dissolution and stood at RT for 20 mins. The mixture was evaporated in vacuo and used for the amide coupling without further purification.

Step 2: The material from step 1 was resuspended in DMF (3 ml), DIPEA (358 mg, 482 μL, 2.77 mmol) was added, followed by 5-hydroxy-6-methylpyrimidine-4-carboxylic acid (89.6 mg, 581 μmol) and the resulting mixture was stirred at RT for 5 mins. HATU (210 mg, 553 μmol) was added and the mixture stirred at RT for 15 mins. The mixture was directly purified by Prep RP HPLC (acidic method, 20-65% ACN). Fractions containing desired product were partitioned between DCM (20 ml) and aqueous NaHCO₃ (5 ml). The organic layer was separated, dried (MgSO₄), filtered and evaporated in vacuo to give the title compound as a beige solid (16 mg, 100% pure).

LC-MS: Rt=1.05 min; MS m/z [M+H]⁺ 728.6/730.5, m/z [M−H]⁻ 726.4/728.4; UPLC-MS 1

LC-MS: Rt=5.12 min; MS m/z [M+H]⁺ 728.1/730.1, m/z [M−H]⁻ 726.3/728.3; UPLC-MS 2

### Example 19A: (R)-6-(4-(4-chloro-3-hydroxypicolinoyl)piperazin-1-yl)-2-(3,6-dihydro-2H-pyran-4-yl)-N-(2-methyl-4-(trifluoromethyl)phenyl)-5-oxo-5,7,8,9-tetrahydropyrrolo[1,2-c][1,2,4]triazolo[1,5-a]pyrimidine-9-carboxamide

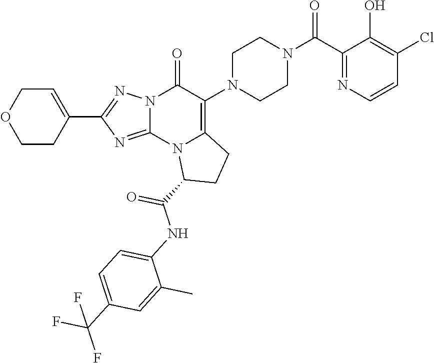 [SMILES: Cc1cc(C(F)(F)F)ccc1NC(=O)[C@H]1CCc2c(N3CCN(C(=O)c4nccc(Cl)c4O)CC3)c(=O)n3nc(C4=CCOCC4)nc3n21]

To a suspension of 4-chloro-3-hydroxypicolinic acid (25.5 mg, 147 μmol) in DCM (1 mL) was added 1-chloro-N,N,2-trimethylprop-1-en-1-amine (22.0 mg, 165 μmol) under argon. The RM was stirred at RT for 2 hours. Then a suspension of (R)-2-(3,6-dihydro-2H-pyran-4-yl)-N-(2-methyl-4-(trifluoromethyl)phenyl)-5-oxo-6-(piperazin-1-yl)-5,7,8,9-tetrahydropyrrolo[1,2-c][1,2,4]triazolo[1,5-a]pyrimidine-9-carboxamide (Intermediate P2) (32.0 mg, 59.0 μmol) was added, followed by DIPEA (41.0 μL, 235 μmol) and the RM was stirred at RT for 1 hour. The RM was diluted with DCM and washed with water. The aqueous layer was extracted twice with DCM. The combined organic layers were dried through a phase separator and concentrated under reduced pressure. The crude product was purified by reverse phase preparative HPLC (RP-HPLC acidic 1: 5 to 100% B in 20 min). The product containing fractions were combined and ACN was removed. The aqueous residue was diluted with 5% aq NaHCO₃ and extracted with DCM. The organic layer was dried through a phase separator and concentrated under reduced pressure. The residue was dissolved with ACN and water, frozen and lyophilized to give the title compound as a white powder (12.5 mg, 95% pure, yield: 29%).

LC-MS: Rt=1.03 min; MS m/z [M+H]⁺ 699.2/701.2, m/z [M−H]⁻ 697.4/699.4; UPLC-MS 1

The stereochemistry of example 19A as (R) was assigned due to its potency based on SAR and structural understanding.

### Example 19B: (S)-6-(4-(4-chloro-3-hydroxypicolinoyl)piperazin-1-yl)-2-(3,6-dihydro-2H-pyran-4-yl)-N-(2-methyl-4-(trifluoromethyl)phenyl)-5-oxo-5,7,8,9-tetrahydropyrrolo[1,2-c][1,2,4]triazolo[1,5-a]pyrimidine-9-carboxamide

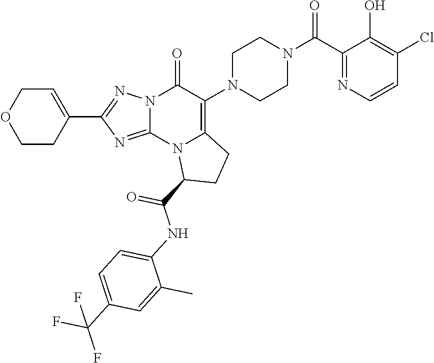 [SMILES: Cc1cc(C(F)(F)F)ccc1NC(=O)[C@@H]1CCc2c(N3CCN(C(=O)c4nccc(Cl)c4O)CC3)c(=O)n3nc(C4=CCOCC4)nc3n21]

To a mixture of (S)-2-(3,6-dihydro-2H-pyran-4-yl)-N-(2-methyl-4-(trifluoromethyl)phenyl)-5-oxo-6-(piperazin-1-yl)-5,7,8,9-tetrahydropyrrolo[1,2-c][1,2,4]triazolo[1,5-a]pyrimidine-9-carboxamide (Intermediate P1) (31.0 mg, 57.0 μmol) and 4-chloro-3-hydroxypicolinoyl chloride (Intermediate Y) (26.0 mg, 114 μmol) in anhydrous THF (1 mL) was added dropwise DIPEA (50.0 μL, 285 μmol). The RM was stirred at RT for 24 hours. To the RM were added 4-chloro-3-hydroxypicolinoyl chloride (Intermediate Y) (19.5 mg, 85.5 μmol) and DIPEA (40.0 μL, 228 μmol). The RM was stirred for additional 24 hours. The RM was diluted with water (20 mL) and extracted with EtOAc (3×15 mL). The combined organic layers were washed with brine, dried over Na₂SO₄, filtered and concentrated under reduced pressure. The crude material was redissolved in DMSO. To the solution were added 4-chloro-3-hydroxypicolinoyl chloride (Intermediate Y) (26.0 mg, 114 μmol) and DIPEA (50.0 μL, 285 μmol) and the RM was stirred at RT over the weekend. The crude product was purified by reverse phase preparative HPLC (RP-HPLC acidic 3: 30 to 60% B in 7 min). The product containing fractions were combined, frozen and lyophilized to give the title compound as a white solid (3.90 mg, 86% pure, yield: 8%).

LC-MS: Rt=1.03 min; MS m/z [M+H]⁺ 699.3/701.3, m/z [M−H]⁻ 697.2/699.2; UPLC-MS 1

### Example 20A: (R)-2-(3,6-dihydro-2H-pyran-4-yl)-6-(4-(3-hydroxypicolinoyl)piperazin-1-yl)-N-(2-methyl-4-(trifluoromethyl)phenyl)-5-oxo-5,7,8,9-tetrahydropyrrolo[1,2-c][1,2,4]triazolo[1,5-a]pyrimidine-9-carboxamide

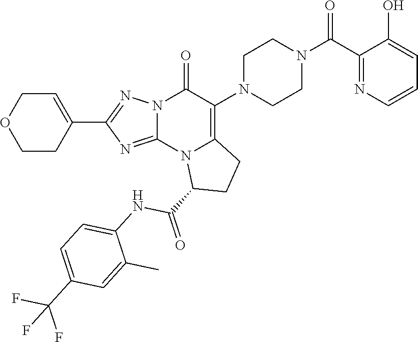 [SMILES: Cc1cc(C(F)(F)F)ccc1NC(=O)[C@H]1CCc2c(N3CCN(C(=O)c4ncccc4O)CC3)c(=O)n3nc(C4=CCOCC4)nc3n21]

(R)-2-(3,6-dihydro-2H-pyran-4-yl)-N-(2-methyl-4-(trifluoromethyl)phenyl)-5-oxo-6-(piperazin-1-yl)-5,7,8,9-tetrahydropyrrolo[1,2-c][1,2,4]triazolo[1,5-a]pyrimidine-9-carboxamide (Intermediate P2) (27 mg, 47 μmol) was dissolved in DCM (Volume: 2 mL) and 3-hydroxy-picolinoyl chloride (Intermediate X) (11 mg, 70 μmol) was added, followed by DIPEA (24 μL, 140 μmol). The mixture was stirred at RT for 2 hours. After 2 and 4 hours, 2 more equivalents of acid chloride and 3 of DIPEA were added. The RM was stirred at RT overnight. The RM was quenched with water and extracted twice with DCM. The organic layer was dried through a phase separator and concentrated under reduced pressure. The crude product was purified by reverse phase preparative HPLC (RP-HPLC acidic 3: 25 to 55% B in 7 min) to give the title compound as a white powder (9 mg, 98% pure, yield: 29%).

LC-MS: Rt=0.95 min; MS m/z [M+H]⁺ 665.5, m/z [M−H]⁻ 663.3; UPLC-MS 1

The stereochemistry of example 20A as (R) was assigned due to its potency based on SAR and structural understanding.

### Example 20B: (S)-2-(3,6-dihydro-2H-pyran-4-yl)-6-(4-(3-hydroxypicolinoyl)piperazin-1-yl)-N-(2-methyl-4-(trifluoromethyl)phenyl)-5-oxo-5,7,8,9-tetrahydropyrrolo[1,2-c][1,2,4]triazolo[1,5-a]pyrimidine-9-carboxamide

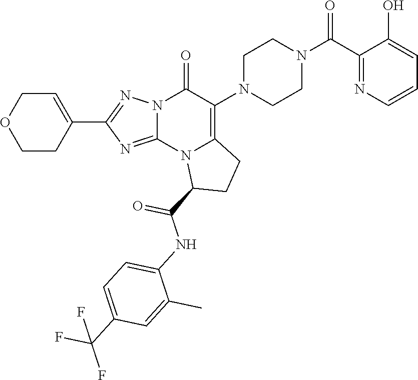 [SMILES: Cc1cc(C(F)(F)F)ccc1NC(=O)[C@@H]1CCc2c(N3CCN(C(=O)c4ncccc4O)CC3)c(=O)n3nc(C4=CCOCC4)nc3n21]

(S)-2-(3,6-dihydro-2H-pyran-4-yl)-N-(2-methyl-4-(trifluoromethyl)phenyl)-5-oxo-6-(piperazin-1-yl)-5,7,8,9-tetrahydropyrrolo[1,2-c][1,2,4]triazolo[1,5-a]pyrimidine-9-carboxamide (Intermediate P1) (27.0 mg, 47.0 μmol) was dissolved in DCM (2 mL) and 3-hydroxypicolinoyl chloride (Intermediate X) (11.0 mg, 70.0 μmol) was added, followed by DIPEA (24.0 μL, 140 μmol). The RM was stirred at RT for 2 hours. 3-Hydroxypicolinoyl chloride (Intermediate X) (14.7 mg, 93.3 μmol) was added, followed by DIPEA (24.0 μL, 140 μmol). The RM was stirred at RT for 2 hours. 3-Hydroxypicolinoyl chloride (Intermediate X) (14.7 mg, 93.3 μmol) was added, followed by DIPEA (24.0 μL, 140 μmol). The RM was stirred at RT overnight. The RM was quenched with water and extracted twice with DCM. The organic layer was dried through a phase separator and concentrated under reduced pressure. The crude product was purified by reverse phase preparative HPLC (RP-HPLC acidic 3: 25 to 55% B in 7 min) to give the title compound as a white powder (9.20 mg, 98% pure, yield: 29%).

LC-MS: Rt=0.95 min; MS m/z [M+H]⁺ 665.5, m/z [M−H]⁻ 663.3; UPLC-MS 1

### Example 21A: (7R,9R)—N-(2-chloro-4-(trifluoromethyl)phenyl)-2-(3,6-dihydro-2H-pyran-4-yl)-6-((1S,6S)-5-(5-hydroxy-6-methylpyrimidine-4-carbonyl)-2,5-diazabicyclo[4.2.0]octan-2-yl)-7-methyl-5-oxo-5,7,8,9-tetrahydropyrrolo[1,2-c][1,2,4]triazolo[1,5-a]pyrimidine-9-carboxamide

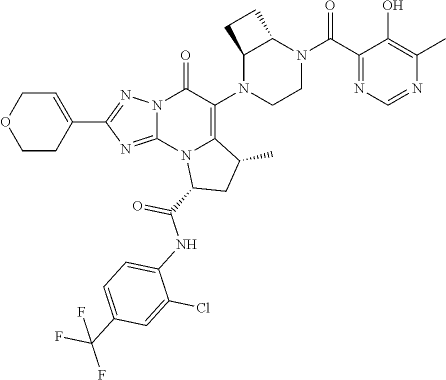 [SMILES: Cc1ncnc(C(=O)N2CCN(c3c4n(c5nc(C6=CCOCC6)nn5c3=O)[C@@H](C(=O)Nc3ccc(C(F)(F)F)cc3Cl)C[C@H]4C)[C@H]3CC[C@@H]32)c1O]

### Step 1: (7R)-6-((1S,6S)-5-(5-(benzyloxy)-6-methylpyrimidine-4-carbonyl)-2,5-diazabicyclo[4.2.0]octan-2-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide

DIPEA (166 μL, 956 μmol) was added at RT to a stirred solution of (7R)-6-((1S,6S)-2,5-diazabicyclo[4.2.0]octan-2-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide (Intermediate Q) (307 mg, 478 μmol), 5-(benzyloxy)-6-methylpyrimidine-4-carboxylic acid (Intermediate U) (128 mg, 526 μmol) and HATU (218 mg, 573 μmol) in DMF (3 mL) and the RM was stirred at RT for 5 minutes. The RM was diluted with EtOAc and water, extracted twice with EtOAc and the combined organic layers were washed with brine, dried over Na₂SO₄ and concentrated under reduced pressure. The crude product was purified by column chromatography (RediSep Column: Silica 24 g, eluent DCM:DCM/MeOH (8/2) 100:0 to 50:50). The product containing fractions were combined and concentrated under reduced pressure to give the title compound (mixture of cis/trans) as a white solid (422 mg, 88% pure, yield: 94%).

LC-MS: Rt=1.25/1.27 min; MS m/z [M+H]⁺ 830.6/832.6, m/z [M−H]⁻ 828.4/830.4; UPLC-MS 1

LC-MS: Rt=5.87/5.98 min; MS m/z [M−H]⁻ 828.4/830.3; UPLC-MS 2

### Step 2: (7R,9R)—N-(2-chloro-4-(trifluoromethyl)phenyl)-2-(3,6-dihydro-2H-pyran-4-yl)-6-((1S,6S)-5-(5-hydroxy-6-methylpyrimidine-4-carbonyl)-2,5-diazabicyclo[4.2.0]octan-2-yl)-7-methyl-5-oxo-5,7,8,9-tetrahydropyrrolo[1,2-c][1,2,4]triazolo[1,5-a]pyrimidine-9-carboxamide

TFA (3.00 mL, 38.9 mmol) was added at RT to (7R)-6-((1S,6S)-5-(5-(benzyloxy)-6-methylpyrimidine-4-carbonyl)-2,5-diazabicyclo[4.2.0]octan-2-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide (422 mg, 447 μmol) and the RM was stirred at RT for 14 hours. The RM was diluted with EtOAc and water, extracted twice with EtOAc and the combined organic layers were washed with brine, dried over Na₂SO₄ and concentrated under reduced pressure. The crude product was purified by column chromatography (RediSep Column: Silica 40 g, eluent DCM:DCM/MeOH (8/2) 100:0 to 50:50). The product containing fractions were combined and concentrated under reduced pressure to give a white foam, which was purified in 2 portions by reverse phase preparative HPLC (RP-HPLC acidic 1: 30 to 70% B in 40 min), (RP-HPLC acidic 1: 30 to 70% B in 40 min). The product containing fractions were combined, basified with aq sat NaHCO₃, ACN was removed under reduced pressure and the aqueous residue was extracted with EtOAc. The combined organic layers were washed with brine, dried over Na₂SO₄ and concentrated under reduced pressure to give a white solid, which was purified by preparative chiral SFC (instrument: Sepiatec Prep SFC100; column: OVEN3 Chiralpak IB-N 250 mm×30 mm 5 μm; eluent: A: 55% 0.05% NH₃ in MeOH, B: 45% scCO₂; flow rate: 60.0 mL/min; detection: UV; injection volume: 2.3 mL; gradient: isocratic A: 55%, B: 45%; oven temperature: 40° C.; BPR: 130 bar) to give the title compound as a white solid (164 mg, 100% pure, yield: 50%). 30 mg of Example 21A title compound (7R,9R)—N-(2-chloro-4-(trifluoromethyl)phenyl)-2-(3,6-dihydro-2H-pyran-4-yl)-6-((1S,6S)-5-(5-hydroxy-6-methylpyrimidine-4-carbonyl)-2,5-diazabicyclo[4.2.0]octan-2-yl)-7-methyl-5-oxo-5,7,8,9-tetrahydropyrrolo[1,2-c][1,2,4]triazolo[1,5-a]pyrimidine-9-carboxamide was dispersed in 40 μl ethanol, then sonicated. The suspension was shaken for 10 minutes at 50° C., then dried at 50° C. under vacuum. X-ray diffraction of the resulting crystals gave the diffractogram according to FIG. 6. The table Ex 21A below shows the most prominent peaks (deg 2theta) of the XRPD diffractogram of FIG. 6.

TABLE 21A

| | | |
|---|---|---|
| Angle 2-theta | D Value | Relative intensity % |

| | | | |
|---|---|---|---|
| 5.77° | 15.3 | Å | 65.5% |
| 6.67° | 13.24 | Å | 20.8% |
| 10.54° | 8.38 | Å | 23.5% |
| 12.36° | 7.15 | Å | 72.3% |
| 12.90° | 6.85 | Å | 55.8% |
| 13.02° | 6.79 | Å | 61.0% |
| 14.83° | 5.96 | Å | 46.3% |
| 15.27° | 5.79 | Å | 56.1% |
| 15.65° | 5.65 | Å | 64.2% |
| 15.99° | 5.53 | Å | 18.3% |
| 17.34° | 5.1 | Å | 71.3% |
| 18.52° | 4.78 | Å | 30.4% |
| 19.18° | 4.62 | Å | 69.7% |
| 20.04° | 4.42 | Å | 88.9% |
| 21.17° | 4.19 | Å | 25.5% |
| 21.47° | 4.13 | Å | 100.0% |
| 21.86° | 4.06 | Å | 42.8% |
| 22.96° | 3.87 | Å | 67.1% |
| 23.18° | 3.83 | Å | 72.9% |
| 23.9° | 3.71 | Å | 97.9% |

LC-MS: Rt=1.16 min; MS m/z [M+H]⁺ 740.4/742.3, m/z [M−H]⁻ 738.3/740.3; UPLC-MS 1

LC-MS: Rt=5.44 min; MS m/z [M+H]⁺ 740.3/742.3, m/z [M−H]⁻ 738.3/740.3; UPLC-MS 2

Chiral HPLC (C-HPLC 5): Rt=2.22 min, 99.5% ee

¹H NMR (600 MHz, DMSO) δ 11.05-10.35 (m, 2H), 8.56 (s, 1H), 7.98 (s, 1H), 7.93 (br d, J=8.4 Hz, 1H), 7.74 (br d, J=8.4 Hz, 1H), 6.79 (br s, 1H), 5.54 (br d, J=9.9 Hz, 1H), 4.43-4.38 (m, 1H) 4.26-4.22 (m, 2H), 3.83-3.77 (m, 3H), 3.72-3.25 (m, 5H), 3.03-2.97 (m, 1H), 2.54-2.48 (m, 2H), 2.43 (s, 3H), 2.10 (br d, J=13.6 Hz, 1H), 1.66-1.60 (m, 1H), 1.49-1.37 (m, 5H), 1.19-1.11 (m, 1H).

The stereochemistry of example 21A was determined by WRN co-crystal X-ray diffraction, and single-crystal X-ray diffraction.

The sodium salt was prepared analogous to the general procedure.

### Example 21C: (7S,9R)—N-(2-chloro-4-(trifluoromethyl)phenyl)-2-(3,6-dihydro-2H-pyran-4-yl)-6-((1S,6S)-5-(5-hydroxy-6-methylpyrimidine-4-carbonyl)-2,5-diazabicyclo[4.2.0]octan-2-yl)-7-methyl-5-oxo-5,7,8,9-tetrahydropyrrolo[1,2-c][1,2,4]triazolo[1,5-a]pyrimidine-9-carboxamide

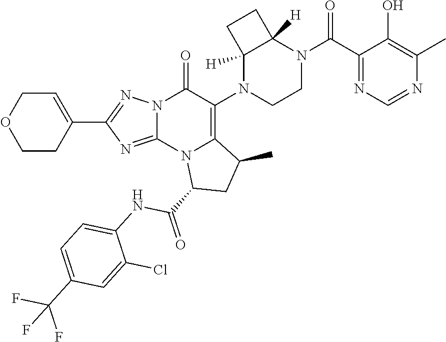 [SMILES: Cc1ncnc(C(=O)N2CCN(c3c4n(c5nc(C6=CCOCC6)nn5c3=O)[C@@H](C(=O)Nc3ccc(C(F)(F)F)cc3Cl)C[C@@H]4C)[C@H]3CC[C@@H]32)c1O]

Example 21C was isolated as first eluting stereoisomer from the preparative HPLC of example 21A

LC-MS: Rt=1.12 min; MS m/z [M+H]⁺ 740.4/742.3, m/z [M−H]⁻ 738.3/740.3; UPLC-MS 1

LC-MS: Rt=5.25 min; MS m/z [M+H]⁺ 740.3/742.3, m/z [M−H]⁻ 738.3/740.3; UPLC-MS 2

### Example 22: rac-6-(4-acetylpiperazin-1-yl)-5-oxo-2-phenyl-N-(4-(trifluoromethyl)phenyl)-5,7,8,9-tetrahydropyrrolo[1,2-c][1,2,4]triazolo[1,5-a]pyrimidine-9-carboxamide

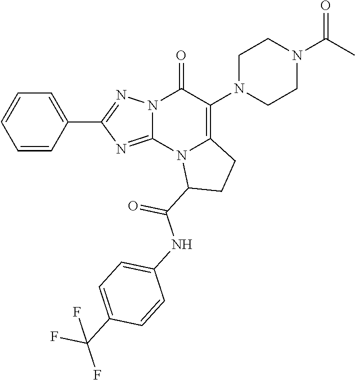 [SMILES: CC(=O)N1CCN(c2c3n(c4nc(-c5ccccc5)nn4c2=O)C(C(=O)Nc2ccc(C(F)(F)F)cc2)CC3)CC1]

Rac-5-oxo-2-phenyl-6-(piperazin-1-yl)-N-(4-(trifluoromethyl)phenyl)-5,7,8,9-tetrahydropyrrolo[1,2-c][1,2,4]triazolo[1,5-a]pyrimidine-9-carboxamide (Intermediate R) (49.0 mg, 94.0 μmol) was mixed with dry THF. Et₃N (27.5 μL, 374 μmol) and acetic anhydride (13.4 μL, 122 μmol) were added. The RM was stirred at 60° C. for 30 minutes. The white precipitate was filtered off and washed with THF, then it was dissolved in DCM. The organic phase was washed 3 times with NaHCO₃ and brine, dried through a phase separator and concentrated under reduced pressure to give the title compound as a white powder (24.4 mg, 95% pure, yield: 44%).

LC-MS: Rt=1.06 min; MS m/z [M+H]⁺ 566.3, MS m/z [M−H]⁻ 564.3; UPLC-MS 3

LC-MS: Rt=5.64 min; MS m/z [M+H]⁺ 566.2, MS m/z [M−H]⁻ 564.1; UPLC-MS 4

### Example 22B: (S)-6-(4-acetylpiperazin-1-yl)-5-oxo-2-phenyl-N-(4-(trifluoromethyl)phenyl)-5,7,8,9-tetrahydropyrrolo[1,2-c][1,2,4]triazolo[1,5-a]pyrimidine-9-carboxamide

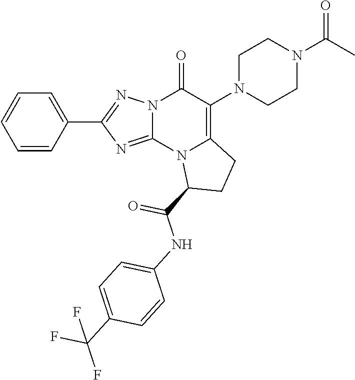 [SMILES: CC(=O)N1CCN(c2c3n(c4nc(-c5ccccc5)nn4c2=O)[C@H](C(=O)Nc2ccc(C(F)(F)F)cc2)CC3)CC1]

Example 22B (12.0 mg, 21.0 μmol) was purified by preparative chiral HPLC of rac-6-(4-acetylpiperazin-1-yl)-5-oxo-2-phenyl-N-(4-(trifluoromethyl)phenyl)-5,7,8,9-tetrahydropyrrolo[1,2-c][1,2,4]triazolo[1,5-a]pyrimidine-9-carboxamide (instrument: Ultimate3000; column: Chiralpak IG-3 (3.0 mm×100 mm 3 μm); eluent: A: 40% heptane with DEA, B: 60% EtOH with DEA; flow rate: 0.420 mL/min; detection: 240 nm; injection volume: 4 mL).

Peak 1: not isolated

Chiral HPLC (C-HPLC 6): Rt=3.62 min, 65% ee

Peak 2: (S)-6-(4-acetylpiperazin-1-yl)-5-oxo-2-phenyl-N-(4-(trifluoromethyl)phenyl)-5,7,8,9-tetrahydropyrrolo[1,2-c][1,2,4]triazolo[1,5-a]pyrimidine-9-carboxamide pale yellow powder (1.70 mg, 97% pure, yield: 14%).

LC-MS: Rt=1.10 min; MS m/z [M+H]⁺ 566.2, m/z [M−H]⁻ 564.1; UPLC-MS 3

Chiral HPLC (C-HPLC 6): Rt=6.86 min, 99.5% ee

The stereochemistry of example 22B as (S) was assigned due to its lower potency than racemic example 22 based SAR and structural understanding.

### Example 23A: (R)-2-(3,6-dihydro-2H-pyran-4-yl)-6-(4-(3-hydroxypicolinoyl)piperazin-1-yl)-5-oxo-N-(4-(trifluoromethyl)phenyl)-5,7,8,9-tetrahydropyrrolo[1,2-c][1,2,4]triazolo[1,5-a]pyrimidine-9-carboxamide

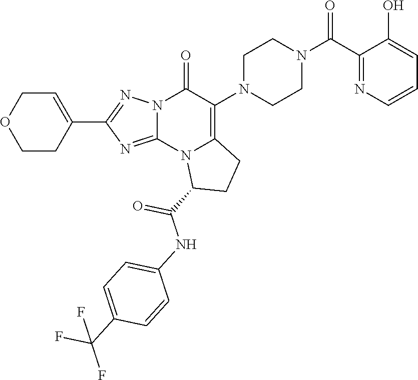 [SMILES: O=C(Nc1ccc(C(F)(F)F)cc1)[C@H]1CCc2c(N3CCN(C(=O)c4ncccc4O)CC3)c(=O)n3nc(C4=CCOCC4)nc3n21]

(R)-2-(3,6-dihydro-2H-pyran-4-yl)-5-oxo-6-(piperazin-1-yl)-N-(4-(trifluoromethyl)phenyl)-5,7,8,9-tetrahydropyrrolo[1,2-c][1,2,4]triazolo[1,5-a]pyrimidine-9-carboxamide (Intermediate T3) (29.0 mg, 51.0 μmol) was dissolved in DCM (2 mL) and 3-hydroxypicolinoyl chloride (Intermediate X) (12.1 mg, 77.0 μmol) was added, followed by DIPEA (27.0 μL, 154 μmol). The RM was stirred at RT for 2 hours. 3-Hydroxypicolinoyl chloride (Intermediate X) (8.07 mg, 51.3 μmol) was added, followed by DIPEA (9.00 μL, 51.3 μmol). The RM was stirred at RT for 2 hours. 3-Hydroxypicolinoyl chloride (Intermediate X) (8.07 mg, 51.3 μmol) was added and the RM was stirred at RT for 1 hour. The reaction was quenched with water and extracted twice with DCM. The organic layer was dried through a phase separator and concentrated under reduced pressure. The crude product was purified by reverse phase preparative HPLC (RP-HPLC acidic 3: 30 to 60% B in 7 min) to give the title compound as a white powder (14.2 mg, 98% pure, yield: 42%).

LC-MS: Rt=0.94 min; MS m/z [M+H]⁺ 651.3, m/z [M−H]⁻ 649.5; UPLC-MS 6

The stereochemistry of example 23A was determined by single-crystal X-ray diffraction and WRN co-crystal X-ray diffraction.

### Example 23B: (S)-2-(3,6-dihydro-2H-pyran-4-yl)-6-(4-(3-hydroxypicolinoyl)piperazin-1-yl)-5-oxo-N-(4-(trifluoromethyl)phenyl)-5,7,8,9-tetrahydropyrrolo[1,2-c][1,2,4]triazolo[1,5-a]pyrimidine-9-carboxamide

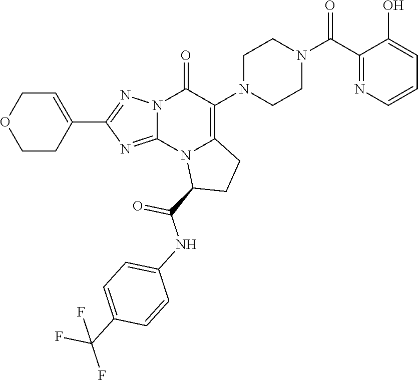 [SMILES: O=C(Nc1ccc(C(F)(F)F)cc1)[C@@H]1CCc2c(N3CCN(C(=O)c4ncccc4O)CC3)c(=O)n3nc(C4=CCOCC4)nc3n21]

(S)-2-(3,6-dihydro-2H-pyran-4-yl)-5-oxo-6-(piperazin-1-yl)-N-(4-(trifluoromethyl)phenyl)-5,7,8,9-tetrahydropyrrolo[1,2-c][1,2,4]triazolo[1,5-a]pyrimidine-9-carboxamide (Intermediate T2) (34.0 mg, 57.0 μmol) was dissolved in DCM (2 mL) and 3-hydroxypicolinoyl chloride (Intermediate X) (13.5 mg, 86.0 μmol) was added, followed by DIPEA (30.0 μL, 171 μmol). The RM was stirred at RT for 2 hours. 3-Hydroxypicolinoyl chloride (Intermediate X) (8.99 mg, 57.3 μmol) was added, followed by DIPEA (10.0 μL, 57.0 μmol). The RM was stirred at RT for 2 hours. 3-Hydroxypicolinoyl chloride (Intermediate X) (8.99 mg, 57.3 μmol) was added and the RM was stirred at RT for 1 hour. The reaction was quenched with water and extracted twice with DCM. The organic layer was dried through a phase separator and concentrated under reduced pressure. The crude product was purified by reverse phase preparative HPLC (RP-HPLC acidic 3: 25 to 55% B in 7 min) to give the title compound as a white powder (14.1 mg, 98% pure, yield: 37%).

LC-MS: Rt=0.94 min; MS m/z [M+H]⁺ 651.4, m/z [M−H]⁻ 649.5; UPLC-MS 6

### Example 24: rac-6-(4-acetylpiperazin-1-yl)-2-(3,6-dihydro-2H-pyran-4-yl)-5-oxo-N-(4-(trifluoromethyl)phenyl)-5,7,8,9-tetrahydropyrrolo[1,2-c][1,2,4]triazolo[1,5-a]pyrimidine-9-carboxamide

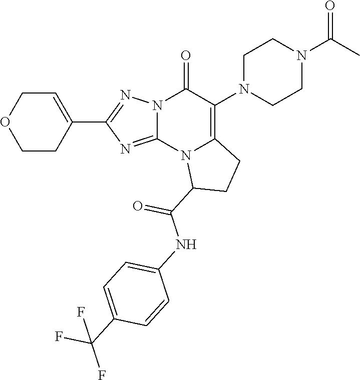 [SMILES: CC(=O)N1CCN(c2c3n(c4nc(C5=CCOCC5)nn4c2=O)C(C(=O)Nc2ccc(C(F)(F)F)cc2)CC3)CC1]

To a solution of rac-2-(3,6-dihydro-2H-pyran-4-yl)-5-oxo-6-(piperazin-1-yl)-N-(4-(trifluoromethyl)phenyl)-5,7,8,9-tetrahydropyrrolo[1,2-c][1,2,4]triazolo[1,5-a]pyrimidine-9-carboxamide (Intermediate T1) (16.0 mg, 27.0 μmol) in DCM (500 μL) were added Et₃N (15.0 μL, 106 μmol), followed by acetic anhydride (2.76 μL, 29.0 μmol). The RM was stirred for 30 minutes. The solution was diluted with DCM and washed with water. The organic layer was dried through a phase separator and concentrated under reduced pressure. The crude product was purified by reverse phase preparative HPLC (RP-HPLC acidic 3: 25 to 55% B in 7 min) to give the title compound as a white powder (7.50 mg, 98% pure, yield: 48%).

LC-MS: Rt=0.91 min; MS m/z [M+H]⁺ 572.4, m/z [M−H]⁻ 570.3; UPLC-MS 6

### Example 25: (7R,9R)—N-(2-chloro-4-(trifluoromethyl)phenyl)-6-((1S,6S)-5-(5-hydroxy-6-methylpyrimidine-4-carbonyl)-2,5-diazabicyclo[4.2.0]octan-2-yl)-7-methyl-5-oxo-2-(6-oxo-3,6-dihydro-2H-pyran-4-yl)-5,7,8,9-tetrahydropyrrolo[1,2-c][1,2,4]triazolo[1,5-a]pyrimidine-9-carboxamide

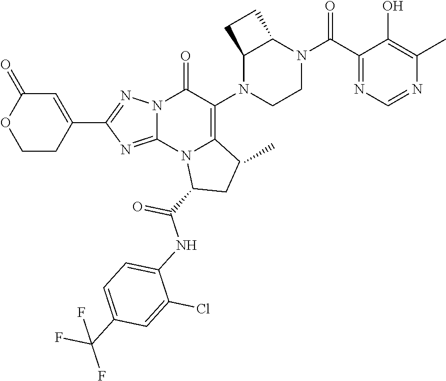

### Step 1: (7R,9R)—N-(2-chloro-4-(trifluoromethyl)phenyl)-6-((1S,6S)-5-(5-hydroxy-6-methylpyrimidine-4-carbonyl)-2,5-diazabicyclo[4.2.0]octan-2-yl)-7-methyl-5-oxo-2-(6-oxo-3,6-dihydro-2H-pyran-4-yl)-5,7,8,9-tetrahydropyrrolo[1,2-c][1,2,4]triazolo[1,5-a]pyrimidine-9-carboxamide

2.60 g (3.51 mmol) of Example 21A (7R,9R)—N-(2-chloro-4-(trifluoromethyl)phenyl)-2-(3,6-dihydro-2H-pyran-4-yl)-6-((1S,6S)-5-(5-hydroxy-6-methylpyrimidine-4-carbonyl)-2,5-diazabicyclo[4.2.0]octan-2-yl)-7-methyl-5-oxo-5,7,8,9-tetrahydropyrrolo[1,2-c][1,2,4]triazolo[1,5-a]pyrimidine-9-carboxamide was stored at RT under ambient atmosphere for several months, by which the purity decreased to 86%. The degradation product title compound Example 25 was isolated by column chromatography (RediSep Column: Silica 80 g, eluent DCM:MeOH 100:0 to 90:10). The product containing fractions were combined and concentrated under reduced pressure to give a yellow foam. This foam was further purified by chiral SFC (instrument: Waters Prep SFC100-MS; column: Chiralpak IB-N 250 mm×30 mm 5 μm; eluent: A: 55% MeOH+0.1% NH₃, B: 45% scCO₂; flow rate: 78.0 mL/min; detection: DAD; injection volume: 1.1 mL; gradient: isocratic A: 55%, B: 45%).

Peak 1: title compound (7R,9R)—N-(2-chloro-4-(trifluoromethyl)phenyl)-6-((1S,6S)-5-(5-hydroxy-6-methylpyrimidine-4-carbonyl)-2,5-diazabicyclo[4.2.0]octan-2-yl)-7-methyl-5-oxo-2-(6-oxo-3,6-dihydro-2H-pyran-4-yl)-5,7,8,9-tetrahydropyrrolo[1,2-c][1,2,4]triazolo[1,5-a]pyrimidine-9-carboxamide

colorless solid (51.0 mg, 97% pure, yield: 2%)

LC-MS: Rt=1.10 min; MS m/z [M+H]⁺ 754.2; UPLC-MS 1

Chiral HPLC (C-HPLC 5): Rt=1.74 min, 98% ee

Peak 2: (7R,9R)—N-(2-chloro-4-(trifluoromethyl)phenyl)-2-(3,6-dihydro-2H-pyran-4-yl)-6-((1S,6S)-5-(5-hydroxy-6-methylpyrimidine-4-carbonyl)-2,5-diazabicyclo[4.2.0]octan-2-yl)-7-methyl-5-oxo-5,7,8,9-tetrahydropyrrolo[1,2-c][1,2,4]triazolo[1,5-a]pyrimidine-9-carboxamide (Example 21A)

colorless solid (1.68 g, 100% pure, yield: 65%)

LC-MS: Rt=1.16 min; MS m/z [M−H]⁻ 738.3/740.3; UPLC-MS 1

Chiral HPLC (C-HPLC 5): Rt=2.24 min, 99% ee

### Preparation of Intermediates

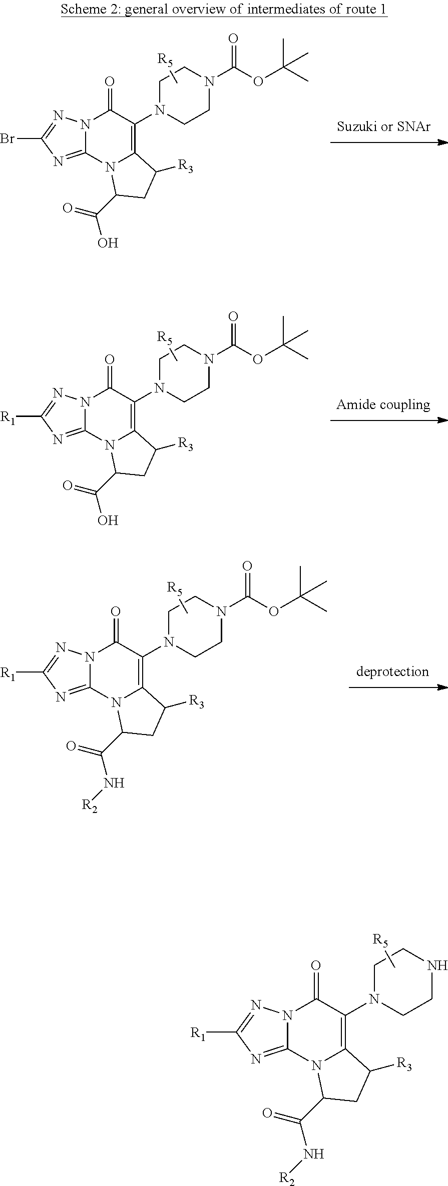

### Intermediate A: tert-butyl 4-(2-bromo-5-ethyl-7-oxo-4,7-dihydro-[1,2,4]triazolo[1,5-a]pyrimidin-6-yl)piperazine-1-carboxylate

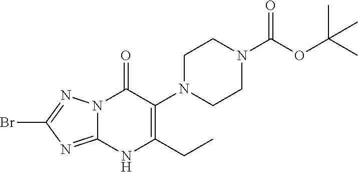 [SMILES: CCc1[nH]c2nc(Br)nn2c(=O)c1N1CCN(C(=O)OC(C)(C)C)CC1]

3-Bromo-1H-1,2,4-triazol-5-amine (Intermediate AI) (82.6 g, 507 mmol) and tert-butyl 4-(1-methoxy-1,3-dioxopentan-2-yl)piperazine-1-carboxylate (Intermediate AC) (175 g, 557 mmol) were mixed in EtOH (465 mL). H₃PO₄ (49.7 g, 507 mmol) was added. The mixture was stirred at 80° C. for 12 hours under nitrogen. The mixture was concentrated under reduced pressure to remove EtOH, then quenched by addition of aq sat NaHCO₃ (1 L), and extracted with DCM (3×1 L). The combined organic layers were washed with brine (3×1 L), dried over Na₂SO₄, filtered and concentrated under reduced pressure to give a residue. The residue was purified by column chromatography (Silica column, eluent DCM:MeOH 1:0 to 10:1). The product containing fractions were combined and concentrated under reduced pressure to give the title compound as a yellow solid (79.0 g, 98% pure, yield: 36%).

LC-MS: Rt=0.91 min; MS m/z [M+H−Boc]⁺ 327.1/329.1, m/z [M+H]⁺ 427.2/429.2, m/z [M−H]⁻ 425.2/427.2; UPLC-MS 1

LC-MS: Rt=4.53 min; MS m/z [M+H−Boc]⁺327.1/329.1, m/z [M−H]⁻ 425.2/427.2; UPLC-MS 2

¹H NMR (400 MHz, DMSO-d₆) δ 13.27 (s, 1H), 3.91 (m, 2H), 3.31 (m, 2H), 2.88 (m, 2H), 2.75 (m, 2H), 2.61 (m, 2H), 1.42 (s, 9H), 1.17 (t, J=7.4 Hz, 3H)

### Intermediate B: 2-bromo-6-(4-(tert-butoxycarbonyl)piperazin-1-yl)-7-methyl-5-oxo-5,7,8,9-tetrahydropyrrolo[1,2-c][1,2,4]triazolo[1,5-a]pyrimidine-9-carboxylic acid

### Step 1: tert-butyl 4-(2-bromo-5-ethyl-7-oxo-4-((2-(trimethylsilyl)ethoxy)methyl)-4,7-dihydro-[1,2,4]triazolo[1,5-a]pyrimidin-6-yl)piperazine-1-carboxylate

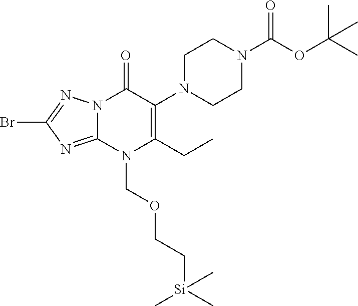 [SMILES: CCc1c(N2CCN(C(=O)OC(C)(C)C)CC2)c(=O)n2nc(Br)nc2n1COCC[Si](C)(C)C]

A yellow solution of tert-butyl 4-(2-bromo-5-ethyl-7-oxo-4,7-dihydro-[1,2,4]triazolo[1,5-a]pyrimidin-6-yl)piperazine-1-carboxylate (Intermediate A) (35.0 g, 82.0 mmol) in THF (650 mL) and DMF (125 mL) was cooled in an ice/methanol bath to −10° C. Then NaH 60% in mineral oil (4.91 g, 123 mmol) was added portion wise within 10 minutes. A strong gas evolution was observed. The RM was stirred at −10° C. for 75 minutes. Then a solution of (2-(chloromethoxy)ethyl)trimethylsilane (17.4 mL, 98.0 mmol) in THF (60 mL) was added dropwise within 30 minutes. The RM was stirred at 0° C. for 3.5 hours. NaH 60% in mineral oil (491 mg, 20.5 mmol) was added and the RM was stirred at 0° C. for 15 minutes. (2-(Chloromethoxy)ethyl)trimethylsilane (2.91 mL, 16.4 mmol) dissolved in THF (10 mL) was added dropwise within 5 minutes. The RM was stirred at 0° C. for 30 minutes. The RM was poured slowly into a stirred mixture of ice water (2 L) and TBME (1.5 L). The organic phase was extracted with water (1 L) and brine (1 L). The aqueous phase was extracted with TBME (500 mL). The combined organic phases were dried over anhydrous Na₂SO₄, filtered and concentrated under reduced pressure at 45° C. to give a pale yellow solid (50.2 g). The crude product was dissolved in DCM (300 mL). Heptane (700 mL) was added slowly. The DCM was distilled off in vacuo at 40° C. bath temperature. After distillation of DCM the yellow solution was seeded with ca 10 mg reference crystals and stirred at RT. The product was crystallized after ca 20-30 minutes. The suspension was stirred at RT for 1 hour and then the pale yellow suspension was cooled in an ice/water bath for another hour. The product was filtered off and the solid was washed with heptane. The solid was dried under HV at 45° C. to give the title compound as white crystals (29.0 g, 99% pure, yield: 63%). The mother liquor was concentrated to give a pale brown oil (19.2 g). The crude product was purified by column chromatography (SNAP Column: Silica 340 g, eluent heptane:EtOAc 90:10 to 30:70). The product containing fractions were combined and concentrated under reduced pressure to give the title compound as a white solid (4.05 g, 99% pure, yield: 9%).

Total: 33.0 g, 99% pure, yield: 72%

LC-MS: Rt=1.50 min; MS m/z [M+H−Boc]⁺ 457.4/459.4, m/z [M+H]⁺ 557.5/559.5; UPLC-MS 1

LC-MS: Rt=7.23 min; MS m/z [M+H−Boc]⁺ 457.4/459.4; UPLC-MS 2

### Step 2: tert-butyl 4-(2-bromo-7-oxo-5-(pent-4-en-2-yl)-4-((2-(trimethylsilyl)ethoxy)methyl)-4,7-dihydro-[1,2,4]triazolo[1,5-a]pyrimidin-6-yl)piperazine-1-carboxylate

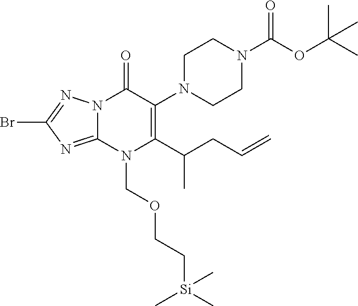 [SMILES: C=CCC(C)c1c(N2CCN(C(=O)OC(C)(C)C)CC2)c(=O)n2nc(Br)nc2n1COCC[Si](C)(C)C]

A pale yellow solution of tert-butyl 4-(2-bromo-5-ethyl-7-oxo-4-((2-(trimethylsilyl)ethoxy)methyl)-4,7-dihydro-[1,2,4]triazolo[1,5-a]pyrimidin-6-yl)piperazine-1-carboxylate (32.5 g, 58.3 mmol) in THF (650 mL) was cooled in a dry ice/aceton bath to −78° C. Then sodium bis(trimethylsilyl)amide 1M in THF (69.9 mL, 69.9 mmol) was added dropwise within 10 minutes. The RM was stirred at −78° C. for 2 hours. A solution of 3-iodoprop-1-ene (13.3 mL, 146 mmol) dissolved in THF (25 mL) was added dropwise within 10 minutes. The RM was stirred at −78° C. overnight. The RM was quenched by dropwise addition of aq sat NH₄Cl (250 mL) at −78° C. Then the RM was allowed to warm to 0° C. within 45 minutes. The RM was extracted with water (600 mL) and TBME (800 mL). The organic phase was extracted with aq sat NaHCO₃ (800 mL) and brine (800 mL). The aqueous phase was extracted with TBME (500 mL). The combined organic layers were dried over anhydrous Na₂SO₄, filtered and concentrated under reduced pressure at 45° C. to give a yellow resin (38.2 g). The crude product was dissolved in DCM (300 mL). Heptane (700 mL) was added slowly. The DCM was distilled off in vacuo at 40° C. bath temperature. The product was crystallized after 30 minutes. The suspension was stirred at RT for 1 hour, then cooled in an ice/water bath for another hour. The solid was filtered off, washed with heptane and dried under HV at 45° C. to give the title compound as white crystals (29.1 g, 99% pure, yield: 83%). The mother liquor was concentrated under reduced pressure to give a yellow oil (6.2 g). The crude product was purified by column chromatography (RediSep Column: Silica 120 g, eluent heptane:EtOAc 90:10 to 30:70). The product containing fractions were combined and concentrated under reduced pressure to give the title compound as a pale yellow foam (4.20 g, 97% pure, yield: 12%).

Total: 33.3 g, 99% pure, yield: 95%

LC-MS: Rt=1.62 min; MS m/z [M+H−Boc]⁺ 497.4/499.4, MS m/z [M+H]⁺ 597.6/599.6; UPLC-MS1

### Step 3: tert-butyl 4-(2-bromo-5-(1-(oxiran-2-yl)propan-2-yl)-7-oxo-4-((2-(trimethylsilyl)ethoxy)methyl)-4,7-dihydro-[1,2,4]triazolo[1,5-a]pyrimidin-6-yl)piperazine-1-carboxylate

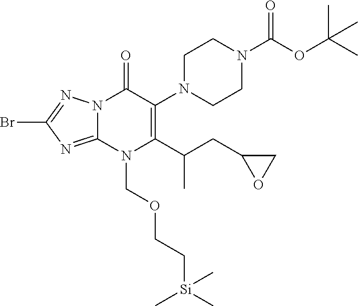 [SMILES: CC(CC1CO1)c1c(N2CCN(C(=O)OC(C)(C)C)CC2)c(=O)n2nc(Br)nc2n1COCC[Si](C)(C)C]

To a colourless solution of tert-butyl 4-(2-bromo-7-oxo-5-(pent-4-en-2-yl)-4-((2-(trimethylsilyl)ethoxy)methyl)-4,7-dihydro-[1,2,4]triazolo[1,5-a]pyrimidin-6-yl)piperazine-1-carboxylate (10.0 g, 16.7 mmol) in DCM (200 mL) was added ethaneperoxoic acid 39% in acetic acid (20.0 mL, 117 mmol). The RM was stirred vigorously at RT for 25 hours. The RM was poured slowly into an ice-cold mixture of aq sat NaHCO₃ (500 mL), aq 10% Na₂S₂O₃ (400 mL) and DCM (250 mL). The mixture was stirred for 5 minutes. Then the phases were separated, the organic phase was washed with aq 5% Na₂S₂O₃ (300 mL) and water (300 mL). The aqueous layers were washed with DCM (2×200 mL). The combined organic layers were dried over anhydrous Na₂SO₄, filtered and concentrated under reduced pressure at 45° C. to give the title compound as a white foam (diastereomeric mixture (racemic)) (10.2 g, 83% pure, yield: 83%).

LC-MS: Rt=1.44 min; MS m/z [M+H−Boc]⁺ 513.4/515.4, MS m/z [M+H]⁺ 613.5/615.5; UPLC-MS9

### Step 4: tert-butyl 4-(2-bromo-9-(hydroxymethyl)-7-methyl-5-oxo-5,7,8,9-tetrahydropyrrolo[1,2-c][1,2,4]triazolo[1,5-a]pyrimidin-6-yl)piperidine-1-carboxylate

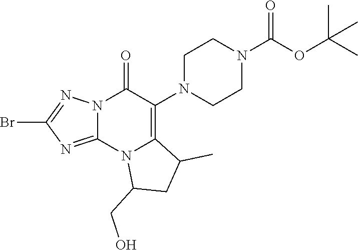 [SMILES: CC1CC(CO)n2c1c(N1CCN(C(=O)OC(C)(C)C)CC1)c(=O)n1nc(Br)nc21]

To a yellow solution of tert-butyl 4-(2-bromo-5-(1-(oxiran-2-yl)propan-2-yl)-7-oxo-4-((2-(trimethylsilyl)ethoxy)methyl)-4,7-dihydro-[1,2,4]triazolo[1,5-a]pyrimidin-6-yl)piperazine-1-carboxylate (31.0 g, 50.5 mmol) in DMF (310 mL) and water (6 mL) was added potassium fluoride (14.7 g, 253 mmol). The RM was stirred at 60° C. overnight. The RM was poured into a stirred mixture of ice water (1.2 L) and TBME (1.2 L). After 2 minutes the phases were separated. The organic layer was washed with water (500 mL) and brine (500 mL). The combined aqueous layers were washed with TBME (2×250 mL). The combined organic layers were dried over anhydrous Na₂SO₄, filtered and concentrated under reduced pressure at 45° C. to give the title compound as a beige solid (diastereomeric mixture (racemic)) (21.6 g, 80% pure, yield: 71%).

LC-MS: Rt=0.87/0.89 min; MS m/z [M+H−Boc]⁺ 383.4/385.4, MS m/z [M−H]⁻ 481.3/483.3; UPLC-MS 1

LC-MS: Rt=4.25/4.37 min; MS m/z [M+H−Boc]⁺383.1/385.1, MS m/z [M−H]⁻ 481.1/483.1; UPLC-MS 2

### Step 5: 2-bromo-6-(4-(tert-butoxycarbonyl)piperazin-1-yl)-7-methyl-5-oxo-5,7,8,9-tetrahydropyrrolo[1,2-c][1,2,4]triazolo[1,5-a]pyrimidine-9-carboxylic acid

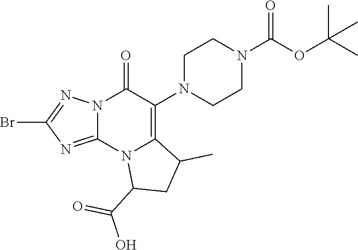 [SMILES: CC1CC(C(=O)O)n2c1c(N1CCN(C(=O)OC(C)(C)C)CC1)c(=O)n1nc(Br)nc21]

To a clear pale brown solution of tert-butyl 4-(2-bromo-9-(hydroxymethyl)-7-methyl-5-oxo-5,7,8,9-tetrahydropyrrolo[1,2-c][1,2,4]triazolo[1,5-a]pyrimidin-6-yl)piperidine-1-carboxylate (21.3 g, 35.3 mmol) in DCM (640 mL) were added aq 5% NaHCO₃ (641 mL, 353 mmol), potassium bromide (420 mg, 3.53 mmol), N-methyl-N,N-dioctyloctan-1-aminium chloride (712 mg, 1.76 mmol) and TEMPO (55.0 mg, 353 μmol). The biphasic RM was cooled in an ice bath to 0° C. Under vigorous stirring aq 5% sodium hypochlorite-solution (109 mL, 88.0 mmol) was added dropwise within 2 hours so that the pH did not rise above 8.6. The RM was stirred for 30 minutes. Aq 5% sodium hypochlorite-solution (65.5 mL, 52.9 mmol) was added dropwise within ca 60 minutes so that the pH did not rise above 8.6. After the addition the RM was stirred at 0° C. for 15 minutes. Aq 5% sodium hypochlorite-solution (43.7 mL, 35.3 mmol) was added dropwise within ca 45 minutes so that the pH did not rise above 8.6. After the addition the RM was stirred at 0° C. for 30 minutes. Aq 5% sodium hypochlorite-solution (21.8 mL, 17.6 mmol) was added dropwise within ca 30 minutes so that the pH did not rise above 8.6. After the addition the RM was stirred at 0° C. for 5 minutes. Aq 5% sodium hypochlorite-solution (21.8 mL, 17.6 mmol) was added dropwise within ca 30 minutes so that the pH did not rise above 8.6. After the addition the RM was stirred at 0° C. for 15 minutes. Aq 5% sodium hypochlorite-solution (21.8 mL, 17.6 mmol) was added dropwise within ca 30 minutes so that the pH did not rise above 8.6. After the addition the RM was stirred at 0° C. for 15 minutes. The RM was diluted with TBME (1.2 L) and stirred vigorously for 2 minutes. Then the phases were separated. The aqueous phase was extracted with TBME (400 mL). The organic phase was extracted with aq 50% NaHCO₃ (400 mL). To the combined water phases was added DCM (500 mL). Then the pH was adjusted under vigorous stirring to pH 3.00 by addition of 5N HCl (200 mL). The mixture was stirred vigorously for 10 minutes at pH 3.00. Then the phases were separated. The aqueous phase was extracted with DCM (3×200 mL). The combined organic layers were dried over anhydrous Na₂SO₄, filtered and concentrated under reduced pressure at 45° C. to give the title compound as a yellowish solid (diastereomeric mixture ca 4:6 (racemic)) (12.2 g, 90% pure, yield: 40%).

LC-MS: Rt=0.72/0.75 min; MS m/z [M+H−Boc]⁺ 397.3/399.3, MS m/z [M−H]⁻ 495.3/497.4; UPLC-MS 1

### Intermediate B: 2-bromo-6-(4-(tert-butoxycarbonyl)piperazin-1-yl)-7-methyl-5-oxo-5,7,8,9-tetrahydropyrrolo[1,2-c][1,2,4]triazolo[1,5-a]pyrimidine-9-carboxylic acid

### Step 1: tert-butyl 4-(2-bromo-5-ethyl-7-oxo-4-((2-(trimethylsilyl)ethoxy)methyl)-4,7-dihydro-[1,2,4]triazolo[1,5-a]pyrimidin-6-yl) piperazine-1-carboxylate

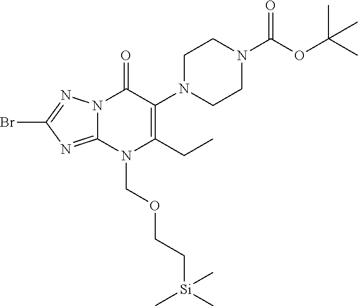 [SMILES: CCc1c(N2CCN(C(=O)OC(C)(C)C)CC2)c(=O)n2nc(Br)nc2n1COCC[Si](C)(C)C]

To a solution of tert-butyl 4-(2-bromo-5-ethyl-7-oxo-4,7-dihydro-[1,2,4]triazolo[1,5-a]pyrimidin-6-yl)piperazine-1-carboxylate (Intermediate A) (286 g, 669 mmol) in THF (5.5 L) and DMF (1.1 L) was added NaH (40.2 g, 1.00 mol) portionwise within 30 minutes at −10° C. under argon. The RM was stirred for 1 hour, then SEMCl (134 g, 803 mmol) in THF (540 mL) was added dropwise within 30 minutes at −10° C. The RM was stirred at 0° C. for 1 hour. NaH (8.00 g, 120 mmol) was added. The RM was stirred for 15 minutes, then SEMCl (40.2 g, 241 mmol) in THF (120 mL) was added. The RM was stirred at 0° C. for 1 hour. The RM was poured slowly into a stirred mixture of ice water (7 L) and TBME (3.5 L). The organic phase was extracted with water (2 L) and brine (2 L). The aqueous phase was extracted with TBME (2 L). The combined organic phases were dried over anhydrous Na₂SO₄, filtered and concentrated under reduced pressure. The crude product was purified by column chromatography (Column: Silica 2 kg, Φ=150 mm, I=800 mm, eluent heptane:EtOAc 100:0 to 20:80). The product containing fractions were combined and concentrated under reduced pressure to give the title compound as a white solid (272 g, 97% pure, yield: 57%).

### Step 2: tert-butyl 4-(2-bromo-7-oxo-5-(pent-4-en-2-yl)-4-((2-(trimethylsilyl)ethoxy)methyl)-4,7-dihydro-[1,2,4]triazolo[1,5-a]pyrimidin-6-yl)piperazine-1-carboxylate

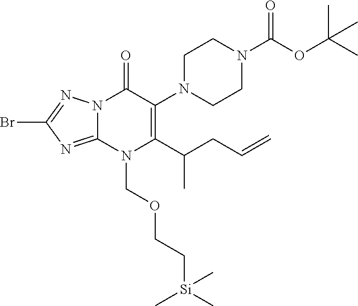 [SMILES: C=CCC(C)c1c(N2CCN(C(=O)OC(C)(C)C)CC2)c(=O)n2nc(Br)nc2n1COCC[Si](C)(C)C]

The solution of tert-butyl 4-(2-bromo-5-ethyl-7-oxo-4-((2-(trimethylsilyl)ethoxy)methyl)-4,7-dihydro-[1,2,4]triazolo[1,5-a]pyrimidin-6-yl)piperazine-1-carboxylate (345 g, 619 mmol) in THF (6.6 L) was cooled to −78° C. under argon, then NaHMDS (371 mL, 742 mmol) was added dropwise and the RM was stirred for 2 hours. Then allyl iodide (250 g, 1.55 mol) in THF (500 mL) was added dropwise and the RM was stirred at −78° C. for 8 hours. The RM was quenched by dropwise addition of aq sat NH₄Cl (2.5 L) at −78° C. Then the RM was allowed to warm to 0° C. The RM was diluted with water (4 L) and extracted with TBME (5 L). The organic phase was washed with aq sat NaHCO₃ (5 L) and brine (5 L). The combined aqueous layers were extracted with TBME (5 L). The combined organic layers were dried over anhydrous Na₂SO₄, filtered and concentrated under reduced pressure. The crude product was dissolved in DCM (3 L). Heptane (7 L) was added slowly dropwise. The DCM was distilled off in vacuo at 40° C. bath temperature. The mixture was stirred at RT for 1 hour and then cooled with an ice/water bath for 1 hour. The solid was filtered off and washed with heptane. The solid was dried under HV at 45° C. to give the title compound. The filtrate was concentrated and purified by column chromatography (Column: Silica 1 kg, Φ=150 mm, I=800 mm, eluent heptane:EtOAc 100:0 to 20:80). The product containing fractions were combined, concentrated under reduced pressure and combined with the first batch to give the title compound as a white solid (294 g, 97% pure, yield: 78%).

### Step 3: tert-butyl 4-(2-bromo-5-(1-(oxiran-2-yl)propan-2-yl)-7-oxo-4-((2-(trimethylsilyl)ethoxy)methyl)-4,7-dihydro-[1,2,4]triazolo[1,5-a]pyrimidin-6-yl)piperazine-1-carboxylate

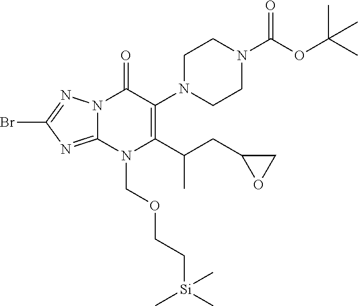 [SMILES: CC(CC1CO1)c1c(N2CCN(C(=O)OC(C)(C)C)CC2)c(=O)n2nc(Br)nc2n1COCC[Si](C)(C)C]

To a solution of tert-butyl 4-(2-bromo-7-oxo-5-(pent-4-en-2-yl)-4-((2-(trimethylsilyl)ethoxy)methyl)-4,7-dihydro-[1,2,4]triazolo[1,5-a]pyrimidin-6-yl)piperazine-1-carboxylate (247 g, 414 mmol) in DCM (5 L) was added peracetic acid ca 18% in acetic acid (838 g, 1.98 mol). The RM was stirred vigorously at RT for 91 hours. The RM was poured slowly into an ice cold mixture of aq sat NaHCO₃ (11 L) and 10% aq Na₂S₂O₃ (8 L). The RM was stirred for 5 minutes. Then the phases were separated, the organic phase was washed with 5% aq Na₂S₂O₃ (6 L) and with 10% brine (6 L). The aqueous layer was extracted with DCM (2×3 L). The combined organic phases were dried over anhydrous Na₂SO₄, filtered and concentrated under reduced pressure to give the title compound as a yellow gelatinous substance (267 g, 85% pure).

### Step 4: tert-butyl 1-(2-bromo-9-(hydroxymethyl)-7-methyl-5-oxo-5,7,8,9-tetrahydropyrrolo[1,2-c][1,2,4]triazolo[1,5-a]pyrimidin-6-yl)piperidine-4-carboxylate

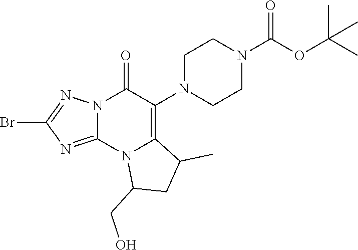 [SMILES: CC1CC(CO)n2c1c(N1CCN(C(=O)OC(C)(C)C)CC1)c(=O)n1nc(Br)nc21]

The solution of tert-butyl 4-(2-bromo-5-(1-(oxiran-2-yl)propan-2-yl)-7-oxo-4-((2-(trimethylsilyl)ethoxy)methyl)-4,7-dihydro-[1,2,4]triazolo[1,5-a]pyrimidin-6-yl)piperazine-1-carboxylate (801 g, 1.30 mol) in DMF (8 L) and water (155 mL) was added KF·2H₂O (630 g, 6.69 mol). The RM was stirred at 60° C. for 28 hours. The RM was poured into a stirred mixture of ice water (32 L) and TBME (32 L). After 2 minutes the phases were separated. The organic phase was washed with water (13 L) and brine (13 L). The combined aqueous layers were washed with TBME (2×6.5 L). The combined organic phases were dried over anhydrous Na₂SO₄, filtered and concentrated under reduced pressure. The residue was triturated with TBME (2 L) and filtered to give the title compound as a yellow solid (400 g, 76% pure, two step yield: 44%).

### Step 5: 2-bromo-6-(4-(tert-butoxycarbonyl)piperazin-1-yl)-7-methyl-5-oxo-5,7,8,9-tetrahydropyrrolo[1,2-c][1,2,4]triazolo[1,5-a]pyrimidine-9-carboxylic acid

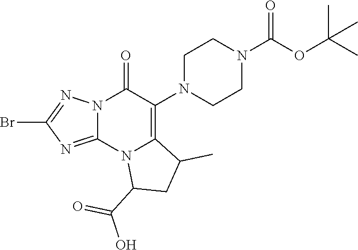 [SMILES: CC1CC(C(=O)O)n2c1c(N1CCN(C(=O)OC(C)(C)C)CC1)c(=O)n1nc(Br)nc21]

The solution of tert-butyl 1-(2-bromo-9-(hydroxymethyl)-7-methyl-5-oxo-5,7,8,9-tetrahydropyrrolo[1,2-c][1,2,4]triazolo[1,5-a]pyrimidin-6-yl)piperidine-4-carboxylate (125 g, 258 mmol) in DCM (3.75 L) was added aq 5% NaHCO₃ (4.00 L, 2.58 mol), potassium bromide (3.00 g, 25.8 mmol), N-methyl-N,N-dioctyloctan-1-aminium chloride (5.20 g, 12.9 mmol) and TEMPO (400 mg, 2.58 mmol). The biphasic RM was cooled to 0° C., then under vigorous stirring 5% sodium hypochlorite-solution (960 g, 645 mmol) was added dropwise within 2 hours. The RM was stirred at 0° C. for 30 minutes. The RM was diluted with DCM (3 L) and stirred vigorously for 2 minutes. Then the phases were separated. The water phase was extracted with DCM (1 L). The organic phase was extracted with aq 50% NaHCO₃ (1 L). To the combined water phases was added DCM (1 L). Then the pH was adjusted under vigorous stirring to pH 3.00 by addition of 5N HCl (1 L). The mixture was stirred vigorously for 10 minutes at pH 3.00. Then the phases were separated. The water phase was extracted with DCM (3×500 mL). The combined organic layers were dried over anhydrous Na₂SO₄, filtered and concentrated under reduced pressure. Three batches were combined and triturated with water (1.5 L) and filtered to give the title compound (diastereomeric mixture ca 6:4 (racemic)) (194 g, yield: 63%).

### Intermediate C: N-(2-chloro-4-(trifluoromethyl)phenyl)-7-methyl-2-morpholino-5-oxo-6-(piperazin-1-yl)-5,7,8,9-tetrahydropyrrolo[1,2-c][1,2,4]triazolo[1,5-a]pyrimidine-9-carboxamide

### Step 1: 6-(4-(tert-butoxycarbonyl)piperazin-1-yl)-7-methyl-2-morpholino-5-oxo-5,7,8,9-tetrahydropyrrolo[1,2-c][1,2,4]triazolo[1,5-a]pyrimidine-9-carboxylic acid

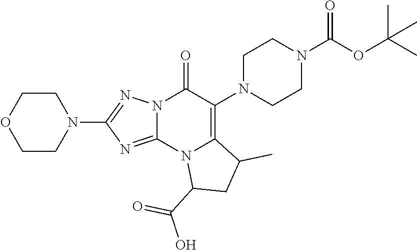 [SMILES: CC1CC(C(=O)O)n2c1c(N1CCN(C(=O)OC(C)(C)C)CC1)c(=O)n1nc(N3CCOCC3)nc21]

2-Bromo-6-(4-(tert-butoxycarbonyl)piperazin-1-yl)-7-methyl-5-oxo-5,7,8,9-tetrahydropyrrolo[1,2-c][1,2,4]triazolo[1,5-a]pyrimidine-9-carboxylic acid (Intermediate B) (4.00 g, 8.04 mmol) and morpholine (3.50 g, 40.2 mmol) were mixed in DMSO (10 mL) and stirred at 150° C. for 1.5 hours. Water (30 mL) and DCM (30 mL) were added. The aqueous layer was washed with DCM (2×20 mL). The aqueous layer still contained product. 1M HCl in water (20 mL) and DCM were added. The aqueous layer was washed with DCM (2×20 mL). The combined organic layers were dried through a phase separator and concentrated under reduced pressure. The crude product was purified by reverse phase preparative ISCO (RediSep Column: C18 240 g, eluent water+0.1% TFA:ACN 90:10 to 65:35 in 30 min with a plateau at 10% for 6 min, at 30% for 3 min and at 35% for 16 min). The product containing fractions were combined, basified with aq sat NaHCO₃, extracted twice with DCM, dried through a phase separator and concentrated under reduced pressure. During concentration a solid collapsed, which was filtered off, washed with a small amount of water and dried under HV to give the title compound (684 mg, 100% pure, yield: 17%). The filtrate was concentrated to dryness and extracted with DCM (2×20 mL). The combined organic layers were dried through a phase separator and concentrated under reduced pressure to give the title compound (2.86 g, 99% pure, yield: 70%).

Total: 3.54 g, 99% pure, yield: 87%.

LC-MS: Rt=0.70/0.73 min; MS m/z [M+H]⁺ 504.4, m/z [M−H]⁻ 502.4; UPLC-MS 1

### Step 2: tert-butyl 4-(9-((2-chloro-4-(trifluoromethyl)phenyl)carbamoyl)-7-methyl-2-morpholino-5-oxo-5,7,8,9-tetrahydropyrrolo[1,2-c][1,2,4]triazolo[1,5-a]pyrimidin-6-yl)piperazine-1-carboxylate

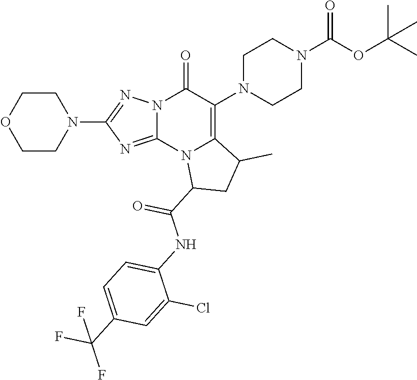 [SMILES: CC1CC(C(=O)Nc2ccc(C(F)(F)F)cc2Cl)n2c1c(N1CCN(C(=O)OC(C)(C)C)CC1)c(=O)n1nc(N3CCOCC3)nc21]

6-(4-(Tert-butoxycarbonyl)piperazin-1-yl)-7-methyl-2-morpholino-5-oxo-5,7,8,9-tetrahydropyrrolo[1,2-c][1,2,4]triazolo[1,5-a]pyrimidine-9-carboxylic acid (2.86 g, 5.67 mmol) and 2-chloro-4-(trifluoromethyl)aniline (1.22 g, 6.24 mmol) were suspended in EtOAc (15 mL). Then Et₃N (13.0 mL, 94.0 mmol) was added and the suspension turned into a solution. T₃P 50% in EtOAc (12.0 mL, 25.2 mmol) was added. The solution was stirred at 50° C. for 1 hour. Water (20 mL), aq sat NaHCO₃ (30 mL) and DCM (10 mL) were added. The aqueous layer was washed with DCM (2×20 mL). The combined organic layers were dried through a phase separator and concentrated under reduced pressure. The crude product was purified by column chromatography (RediSep Column: Silica 80 g, eluent DCM:DCM/MeOH (1/1) 100:0 to 0:100). The product containing fractions were combined, concentrated under reduced pressure and dried under HV to give a brown foam, which was purified by reverse phase preparative ISCO (RediSep Column: C18 240 g, eluent water+0.1% TFA:ACN 90:10 to 0:100 in 29 min with a plateau at 47% for 4 min and at 70% for 10 min). The product containing fractions were combined, basified with aq sat NaHCO₃, extracted twice with DCM, dried through a phase separator and concentrated under reduced pressure to give the title compound (1.99 g, 95% pure, yield: 49%).

LC-MS: Rt=1.25/1.27 min; MS m/z [M+H]⁺ 681.0/683.5, m/z [M−H]⁻ 679.4/681.4; UPLC-MS 1

### Step 3: N-(2-chloro-4-(trifluoromethyl)phenyl)-7-methyl-2-morpholino-5-oxo-6-(piperazin-1-yl)-5,7,8,9-tetrahydropyrrolo[1,2-c][1,2,4]triazolo[1,5-a]pyrimidine-9-carboxamide

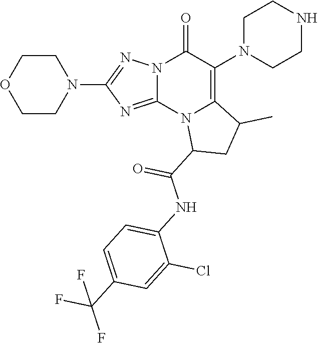 [SMILES: CC1CC(C(=O)Nc2ccc(C(F)(F)F)cc2Cl)n2c1c(N1CCNCC1)c(=O)n1nc(N3CCOCC3)nc21]

Tert-butyl 4-(9-((2-chloro-4-(trifluoromethyl)phenyl)carbamoyl)-7-methyl-2-morpholino-5-oxo-5,7,8,9-tetrahydropyrrolo[1,2-c][1,2,4]triazolo[1,5-a]pyrimidin-6-yl)piperazine-1-carboxylate (1.99 g, 2.77 mmol) was dissolved in DCM (10 mL) and TFA (5.00 mL, 64.9 mmol). The mixture was stirred at RT for 1 hour. Water (10 mL), aq 1M NaOH (20 mL) and DCM (30 mL) were added. The aqueous layer was washed with DCM (2×20 mL) and EtOAc (2×20 mL). The combined organic layers were dried through a phase separator and concentrated under reduced pressure. The crude product was purified in 2 portions by reverse phase preparative ISCO (RediSep Column: C18 50 g gold, eluent water+0.1% TFA:ACN 90:10 to 65:35 in 21 min), (RediSep Column: C18 86 g, eluent water+0.1% TFA:ACN 90:10 to 60:40 in 29 min). The product containing fractions were combined, basified with aq sat NaHCO₃, extracted twice with DCM, dried through a phase separator and concentrated under reduced pressure to give the title compound (1.34 g, 98% pure, yield: 81.5%).

LC-MS: Rt=0.72/0.74 min; MS m/z [M+H]⁺ 581.5/583.5, m/z [M−H]⁻ 579.1/581.1; UPLC-MS 1

### Intermediate D: (7R,9R)—N-(2-chloro-4-(trifluoromethyl)phenyl)-7-methyl-2-morpholino-5-oxo-6-(piperazin-1-yl)-5,7,8,9-tetrahydropyrrolo[1,2-c][1,2,4]triazolo[1,5-a]pyrimidine-9-carboxamide and (7S,9S)—N-(2-chloro-4-(trifluoromethyl)phenyl)-7-methyl-2-morpholino-5-oxo-6-(piperazin-1-yl)-5,7,8,9-tetrahydropyrrolo[1,2-c][1,2,4]triazolo[1,5-a]pyrimidine-9-carboxamide

### Step 1: 6-(4-(tert-butoxycarbonyl)piperazin-1-yl)-7-methyl-2-morpholino-5-oxo-5,7,8,9-tetrahydropyrrolo[1,2-c][1,2,4]triazolo[1,5-a]pyrimidine-9-carboxylic acid

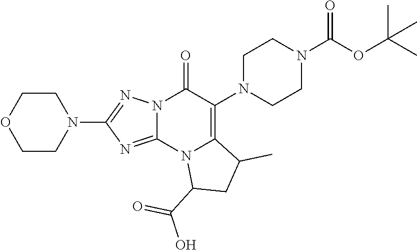 [SMILES: CC1CC(C(=O)O)n2c1c(N1CCN(C(=O)OC(C)(C)C)CC1)c(=O)n1nc(N3CCOCC3)nc21]

2-Bromo-6-(4-(tert-butoxycarbonyl)piperazin-1-yl)-7-methyl-5-oxo-5,7,8,9-tetrahydropyrrolo[1,2-c][1,2,4]triazolo[1,5-a]pyrimidine-9-carboxylic acid (Intermediate B) (10.0 g, 20.1 mmol) and morpholine (8.76 g, 101 mmol) were mixed in DMSO (5 mL) and stirred at 150° C. for 2.5 hours. Water (30 mL) and DCM (30 mL) were added. The aqueous layer was washed with DCM (2×20 mL). The aqueous layer still contained product. 1M HCl in water (20 mL) and DCM (30 mL) were added. The aqueous layer was washed with DCM (2×20 mL). The combined organic layers were dried through a phase separator and concentrated under reduced pressure. The crude product was purified by reverse phase preparative ISCO (RediSep Column: C18 240 g, eluent water+0.1% TFA:ACN 90:10 to 25:75 in 25 min with a plateau at 10% for 7 min, at 28% for 4 min and at 40% for 4 min). The product containing fractions were combined, extracted twice with DCM, dried through a phase separator and concentrated under reduced pressure to give the title compound (8.13 g, 99% pure, yield: 79.5%).

LC-MS: Rt=0.70/0.73 min; MS m/z [M+H]⁺ 504.4, m/z [M−H]⁻ 502.3; UPLC-MS 1

### Step 2: tert-butyl 4-((7R,9R)-9-((2-chloro-4-(trifluoromethyl)phenyl)carbamoyl)-7-methyl-2-morpholino-5-oxo-5,7,8,9-tetrahydropyrrolo[1,2-c][1,2,4]triazolo[1,5-a]pyrimidin-6-yl)piperazine-1-carboxylate and tert-butyl 4-((7S,9S)-9-((2-chloro-4-(trifluoromethyl)phenyl)carbamoyl)-7-methyl-2-morpholino-5-oxo-5,7,8,9-tetrahydropyrrolo[1,2-c][1,2,4]triazolo[1,5-a]pyrimidin-6-yl)piperazine-1-carboxylate

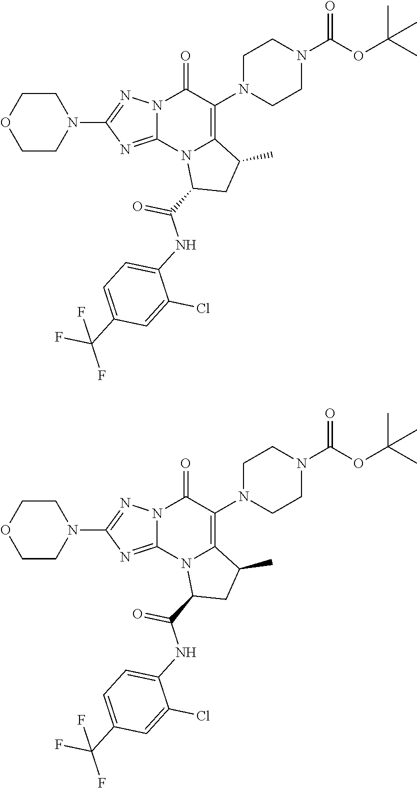 [SMILES: C[C@@H]1C[C@H](C(=O)Nc2ccc(C(F)(F)F)cc2Cl)n2c1c(N1CCN(C(=O)OC(C)(C)C)CC1)c(=O)n1nc(N3CCOCC3)nc21 ; C[C@H]1C[C@@H](C(=O)Nc2ccc(C(F)(F)F)cc2Cl)n2c1c(N1CCN(C(=O)OC(C)(C)C)CC1)c(=O)n1nc(N3CCOCC3)nc21]

6-(4-(Tert-butoxycarbonyl)piperazin-1-yl)-7-methyl-2-morpholino-5-oxo-5,7,8,9-tetrahydropyrrolo[1,2-c][1,2,4]triazolo[1,5-a]pyrimidine-9-carboxylic acid (2.50 g, 4.82 mmol) and 2-chloro-4-(trifluoromethyl)aniline (1.04 g, 5.30 mmol) were dissolved in DCM (5 mL) and Et₃N (10.7 mL, 77.0 mmol) was added, followed by T₃P 50% in EtOAc (22.9 mL, 38.5 mmol). The mixture was stirred at RT for 6 hours. Water (20 mL), aq 1M HCl (20 mL) and DCM (30 mL) were added. The aqueous layer was washed with DCM (2×20 mL). The combined organic layers were dried through a phase separator and concentrated under reduced pressure. The crude product was purified by column chromatography (RediSep Column: Silica 120 g, eluent DCM:DCM/MeOH (1/1) 100:0 to 85:15). The product containing fractions were combined, concentrated under reduced pressure and dried under HV, then it was purified in 2 portions by reverse phase preparative HPLC (RP-HPLC acidic 2: 40 to 70% B in 18 min with a plateau at 65% for 1 min), (RP-HPLC acidic 2: 40 to 70% B in 18 min with a plateau at 65% for 1 min). All fractions containing peak 2 with purity>99% were combined and lyophilized overnight to give the title compound (540 mg, 99% pure, yield: 14%).

LC-MS: Rt=1.25 min; MS m/z [M+H]⁺ 681.5/683.6, m/z [M−H]⁻ 679.2/681.2; UPLC-MS 1

### Step 3: (7R,9R)—N-(2-chloro-4-(trifluoromethyl)phenyl)-7-methyl-2-morpholino-5-oxo-6-(piperazin-1-yl)-5,7,8,9-tetrahydropyrrolo[1,2-c][1,2,4]triazolo[1,5-a]pyrimidine-9-carboxamide and (7S,9S)—N-(2-chloro-4-(trifluoromethyl)phenyl)-7-methyl-2-morpholino-5-oxo-6-(piperazin-1-yl)-5,7,8,9-tetrahydropyrrolo[1,2-c][1,2,4]triazolo[1,5-a]pyrimidine-9-carboxamide

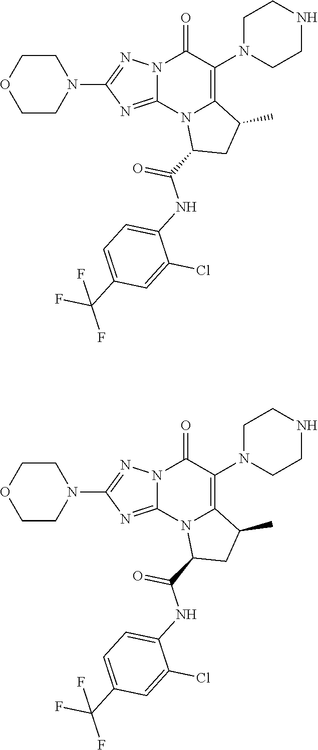 [SMILES: C[C@@H]1C[C@H](C(=O)Nc2ccc(C(F)(F)F)cc2Cl)n2c1c(N1CCNCC1)c(=O)n1nc(N3CCOCC3)nc21 ; C[C@H]1C[C@@H](C(=O)Nc2ccc(C(F)(F)F)cc2Cl)n2c1c(N1CCNCC1)c(=O)n1nc(N3CCOCC3)nc21]

The racemic mixture of tert-butyl 4-((7R,9R)-9-((2-chloro-4-(trifluoromethyl)phenyl)carbamoyl)-7-methyl-2-morpholino-5-oxo-5,7,8,9-tetrahydropyrrolo[1,2-c][1,2,4]triazolo[1,5-a]pyrimidin-6-yl)piperazine-1-carboxylate and tert-butyl 4-((7S,9S)-9-((2-chloro-4-(trifluoromethyl)phenyl)carbamoyl)-7-methyl-2-morpholino-5-oxo-5,7,8,9-tetrahydropyrrolo[1,2-c][1,2,4]triazolo[1,5-a]pyrimidin-6-yl)piperazine-1-carboxylate (540 mg, 674 μmol) was dissolved in DCM (5 mL) and TFA (500 μL, 6.49 mmol) was added. The mixture was stirred at 40° C. for 1.5 hours. The mixture was concentrated under reduced pressure. The crude product was purified by reverse phase preparative HPLC (RP-HPLC acidic 2: 15 to 45% B in 17 min). The product containing fractions were combined, basified with aq sat NaHCO₃, extracted twice with DCM, dried through a phase separator and concentrated under reduced pressure to give the title compound (483 mg, 93% pure, yield: 96%).

LC-MS: Rt=0.73 min; MS m/z [M+H]⁺ 581.5/583.4, m/z [M−H]⁻ 579.0/581.0; UPLC-MS 1

### Intermediate E1: (7R,9R)—N-(2-chloro-6-(trifluoromethyl)pyridin-3-yl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide and Intermediate E2: (7S,9S)—N-(2-chloro-6-(trifluoromethyl)pyridin-3-yl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide

### Step 1: 6-(4-(tert-butoxycarbonyl)piperazin-1-yl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylic acid

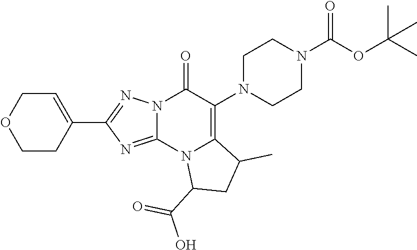 [SMILES: CC1CC(C(=O)O)n2c1c(N1CCN(C(=O)OC(C)(C)C)CC1)c(=O)n1nc(C3=CCOCC3)nc21]

To a stirred solution of 2-bromo-6-(4-(tert-butoxycarbonyl)piperazin-1-yl)-7-methyl-5-oxo-5,7,8,9-tetrahydropyrrolo[1,2-c][1,2,4]triazolo[1,5-a]pyrimidine-9-carboxylic acid (Intermediate B) (10.0 g, 20.1 mmol), K₃PO₄ (12.8 g, 60.3 mmol) and PdCl₂(dppf)·DCM adduct (821 mg, 1.01 mmol) in 1,4-dioxane (200 mL) and water (100 mL) was added at 80° C. 2-(3,6-dihydro-2H-pyran-4-yl)-4,4,5,5-tetramethyl-1,3,2-dioxaborolane (5.49 g, 26.1 mmol) and the RM was stirred at 80° C. for 5 minutes. The RM was diluted with EtOAc and water, extracted twice with EtOAc and the aqueous layer was acidified to pH 2 with 2N HCl. The aqueous layer was extracted twice with EtOAc and the combined organic layers were washed with brine, dried over Na₂SO₄ and concentrated under reduced pressure to give the title compound as a beige foam (7.51 g, 91% pure, yield: 68%).

LC-MS: Rt=0.74/0.76 min; MS m/z [M+H−Boc]⁺ 401.4, m/z [M−H]⁻ 499.4; UPLC-MS 1

### Step 2: tert-butyl 4-((7R,9R)-9-((2-chloro-6-(trifluoromethyl)pyridin-3-yl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate and tert-butyl 4-((7S,9S)-9-((2-chloro-6-(trifluoromethyl)pyridin-3-yl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate

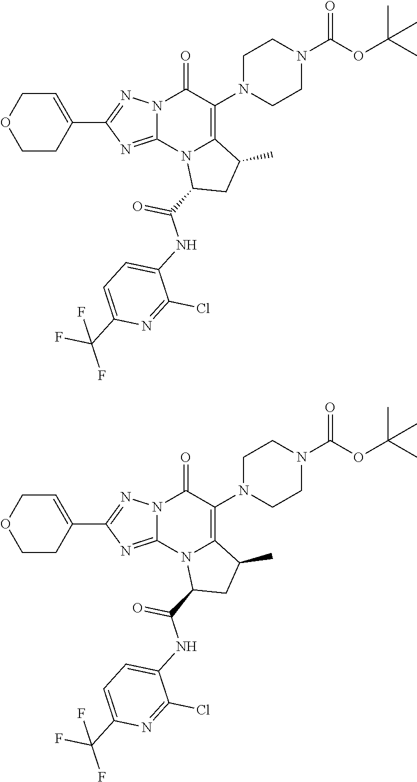 [SMILES: C[C@@H]1C[C@H](C(=O)Nc2ccc(C(F)(F)F)nc2Cl)n2c1c(N1CCN(C(=O)OC(C)(C)C)CC1)c(=O)n1nc(C3=CCOCC3)nc21 ; C[C@H]1C[C@@H](C(=O)Nc2ccc(C(F)(F)F)nc2Cl)n2c1c(N1CCN(C(=O)OC(C)(C)C)CC1)c(=O)n1nc(C3=CCOCC3)nc21]

To a stirred solution of 6-(4-(tert-butoxycarbonyl)piperazin-1-yl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylic acid (2.00 g, 3.16 mmol), 2-chloro-6-(trifluoromethyl)pyridin-3-amine (682 mg, 3.47 mmol) in DCM (40 mL) was added Et₃N (3.52 mL, 25.3 mmol), followed by T₃P 50% in EtOAc (3.76 mL, 6.31 mmol) at RT and the RM was stirred at RT for 14 hours. Then T₃P 50% in EtOAc (1.88 mL, 3.16 mmol) was added and the RM was stirred at RT overnight. The RM was diluted with DCM and aq NaHCO₃, extracted twice with DCM and the combined organic layers were washed with water and brine, dried over Na₂SO₄ and concentrated under reduced pressure. The crude product was purified by column chromatography (RediSep Column: Silica 80 g, eluent DCM:EtOAc 100:0 to 0:100). The product containing fractions were combined, concentrated under reduced pressure and dried under HV to give a beige solid, which was purified in 2 portions by reverse phase preparative HPLC (RP-HPLC basic 1: 5 to 95% B in 20 min), (RP-HPLC basic 1: 5 to 95% B in 20 min) to give the title compound as a white solid (350 mg, 90% pure, yield: 15%).

The mixture (350 mg) was purified twice by preparative chiral SFC (instrument: Sepiatec Prep SFC100; column: Lux-i Cellulose 5 (Chiralpak IC) 250×30 mm 5 μm; eluent: A: 45% MeOH+0.1% NH₃, B: 55% scCO₂; flow rate: 90 mL/min; detection: UV; injection volume: 0.8 mL; gradient: isocratic A: 45%, B: 55%; oven temperature: 40° C.; BPR: 105 bar) and (instrument: Sepiatec Prep SFC100; column: Chiralpak IB (30 mm×250 mm 5 μm); eluent: A: 40% MeOH+0.1% NH₃, B: 60% scCO₂; flow rate: 85 mL/min; detection: UV; injection volume: 1 mL; gradient: isocratic A: 45%, B: 55%; oven temperature: 40° C.; BPR: 110 bar).

Peak 1: tert-butyl 4-((7R,9S)-9-((2-chloro-6-(trifluoromethyl)pyridin-3-yl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate or tert-butyl 4-((7S,9R)-9-((2-chloro-6-(trifluoromethyl)pyridin-3-yl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate 40.0 mg, 95% pure, yield: 11%

Chiral HPLC (C-HPLC 7): Rt=1.70 min, 99.5% ee

LC-MS: Rt=1.18 min; MS m/z [M+H−Boc]⁺ 579.4/581.5, m/z [M−H]⁻ 677.2/679.2; UPLC-MS 1

Peak 2: tert-butyl 4-((7R,9R)-9-((2-chloro-6-(trifluoromethyl)pyridin-3-yl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate 150 mg, 95% pure, yield: 41%

Chiral HPLC (C-HPLC 7): Rt=2.16 min, 99.5% ee

LC-MS: Rt=1.20 min; MS m/z [M+H−Boc]⁺579.4/580.9, m/z [M−H]⁻ 677.2/679.2; UPLC-MS 1

Peak 3: tert-butyl 4-((7S,9S)-9-((2-chloro-6-(trifluoromethyl)pyridin-3-yl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate 145 mg, 95% pure, yield: 39%

Chiral HPLC (C-HPLC 7): Rt=2.84 min, 99.5% ee

LC-MS: Rt=1.20 min; MS m/z [M+H−Boc]⁺ 579.4/581.4, m/z [M−H]⁻ 677.4/679.4; UPLC-MS 1

Peak 4: tert-butyl 4-((7R,9S)-9-((2-chloro-6-(trifluoromethyl)pyridin-3-yl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate or tert-butyl 4-((7S,9R)-9-((2-chloro-6-(trifluoromethyl)pyridin-3-yl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate 32.0 mg, 95% pure, yield: 9%

Chiral HPLC (C-HPLC 7): Rt=3.07 min, 99.5% ee

LC-MS: Rt=1.19 min; MS m/z [M+H−Boc]⁺579.4/581.4, m/z [M−H]⁻ 677.4/679.4; UPLC-MS 1

### Step 3: (7R,9R)—N-(2-chloro-6-(trifluoromethyl)pyridin-3-yl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide and (7S,9S)—N-(2-chloro-6-(trifluoromethyl)pyridin-3-yl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide

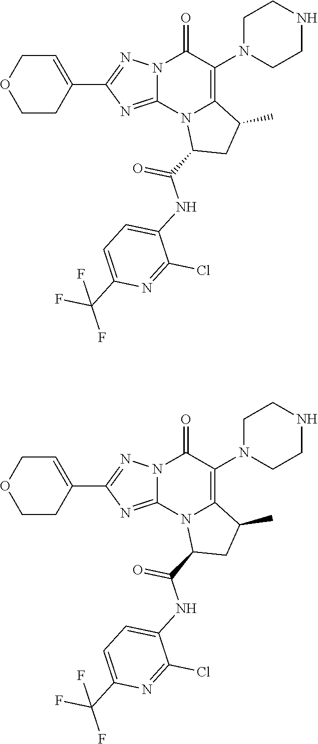 [SMILES: C[C@@H]1C[C@H](C(=O)Nc2ccc(C(F)(F)F)nc2Cl)n2c1c(N1CCNCC1)c(=O)n1nc(C3=CCOCC3)nc21 ; C[C@H]1C[C@@H](C(=O)Nc2ccc(C(F)(F)F)nc2Cl)n2c1c(N1CCNCC1)c(=O)n1nc(C3=CCOCC3)nc21]

Intermediate E1: Tert-butyl 4-((7R,9R)-9-((2-chloro-6-(trifluoromethyl)pyridin-3-yl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate (140 mg, 206 μmol) was dissolved in 4M HCl in 1,4-dioxane (1.50 mL, 6.00 mmol) and stood at RT for 30 minutes. The RM was concentrated under reduced pressure to give the title compound hydrochloride salt as an off-white solid (127 mg, 95% pure, yield: 95%).

LC-MS: Rt=0.63 min; MS m/z [M+H−Boc]⁺ 579.4/581.4, m/z [M−H]⁻ 577.2/578.9; UPLC-MS 1

Intermediate E2: Tert-butyl 4-((7S,9S)-9-((2-chloro-6-(trifluoromethyl)pyridin-3-yl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate (135 mg, 199 μmol) was dissolved in 4M HCl in 1,4-dioxane (1.50 mL, 6.00 mmol) and stood at RT for 30 minutes. The RM was concentrated under reduced pressure to give the title compound hydrochloride salt as an off-white solid (122 mg, 95% pure, yield: 91%).

LC-MS: Rt=0.64 min; MS m/z [M+H−Boc]⁺579.2/581.2, m/z [M−H]⁻ 577.3/579.2; UPLC-MS 1

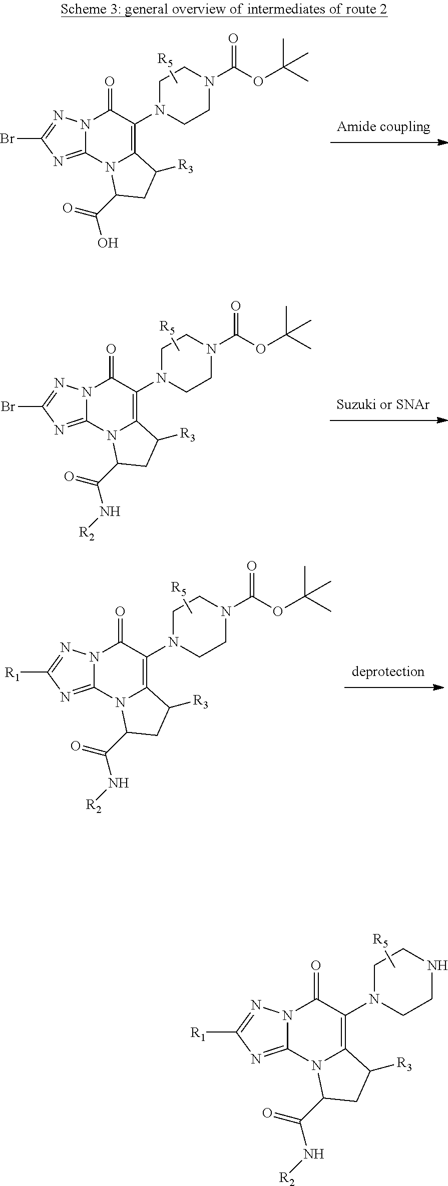

### Intermediate F1: tert-butyl 4-((7R,9S)-2-bromo-9-((2-chloro-4-(trifluoromethyl)phenyl)carbamoyl)-7-methyl-5-oxo-5,7,8,9-tetrahydropyrrolo[1,2-c][1,2,4]triazolo[1,5-a]pyrimidin-6-yl)piperazine-1-carboxylate and tert-butyl 4-((7S,9R)-2-bromo-9-((2-chloro-4-(trifluoromethyl)phenyl)carbamoyl)-7-methyl-5-oxo-5,7,8,9-tetrahydropyrrolo[1,2-c][1,2,4]triazolo[1,5-a]pyrimidin-6-yl)piperazine-1-carboxylate and Intermediate F2: tert-butyl 4-((7S,9S)-2-bromo-9-((2-chloro-4-(trifluoromethyl)phenyl)carbamoyl)-7-methyl-5-oxo-5,7,8,9-tetrahydropyrrolo[1,2-c][1,2,4]triazolo[1,5-a]pyrimidin-6-yl)piperazine-1-carboxylate and tert-butyl 4-((7R,9R)-2-bromo-9-((2-chloro-4-(trifluoromethyl)phenyl)carbamoyl)-7-methyl-5-oxo-5,7,8,9-tetrahydropyrrolo[1,2-c][1,2,4]triazolo[1,5-a]pyrimidin-6-yl)piperazine-1-carboxylate

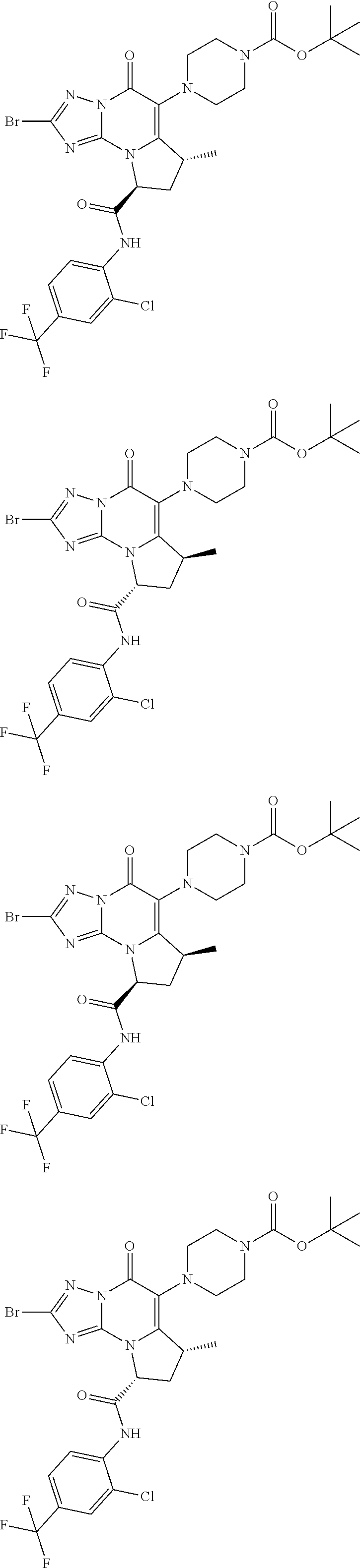 [SMILES: C[C@@H]1C[C@@H](C(=O)Nc2ccc(C(F)(F)F)cc2Cl)n2c1c(N1CCN(C(=O)OC(C)(C)C)CC1)c(=O)n1nc(Br)nc21 ; C[C@H]1C[C@H](C(=O)Nc2ccc(C(F)(F)F)cc2Cl)n2c1c(N1CCN(C(=O)OC(C)(C)C)CC1)c(=O)n1nc(Br)nc21 ; C[C@H]1C[C@@H](C(=O)Nc2ccc(C(F)(F)F)cc2Cl)n2c1c(N1CCN(C(=O)OC(C)(C)C)CC1)c(=O)n1nc(Br)nc21 ; C[C@@H]1C[C@H](C(=O)Nc2ccc(C(F)(F)F)cc2Cl)n2c1c(N1CCN(C(=O)OC(C)(C)C)CC1)c(=O)n1nc(Br)nc21]

To a stirred solution of 2-bromo-6-(4-(tert-butoxycarbonyl)piperazin-1-yl)-7-methyl-5-oxo-5,7,8,9-tetrahydropyrrolo[1,2-c][1,2,4]triazolo[1,5-a]pyrimidine-9-carboxylic acid (Intermediate B) (15.0 g, 30.2 mmol), 2-chloro-4-(trifluoromethyl)aniline (4.58 mL, 33.2 mmol) in DCM (50 mL) were added Et₃N (33.6 mL, 241 mmol) and T₃P 50% in EtOAc (35.9 mL, 60.3 mmol) at 0° C. and the RM was stirred at RT for 1 hour, then T₃P 50% in EtOAc (18.0 mL, 30.2 mmol) was added and the RM was stirred at RT overnight. The RM was diluted with DCM and aq NaHCO₃, extracted twice with DCM and the combined organic layers were washed with water and brine, dried over Na₂SO₄ and concentrated under reduced pressure. The crude product was triturated in EtOAc to afford a white solid, mainly Peak 2 (4.80 g, 93% pure, yield: 22%). The filtrate was concentrated and purified by column chromatography (RediSep Column: Silica 330 g, eluent heptane:EtOAc 80:20 to 0:100).

Intermediate F1: All pure fractions containing peak 1 were combined, concentrated under reduced pressure and dried under HV to give the title compound as a racemic mixture of tert-butyl 4-((7R,9S)-2-bromo-9-((2-chloro-4-(trifluoromethyl) phenyl)carbamoyl)-7-methyl-5-oxo-5,7,8,9-tetrahydropyrrolo[1,2-c][1,2,4]triazolo[1,5-a]pyrimidin-6-yl)piperazine-1-carboxylate and tert-butyl 4-((7S,9R)-2-bromo-9-((2-chloro-4-(trifluoromethyl)phenyl)carbamoyl)-7-methyl-5-oxo-5,7,8,9-tetrahydropyrrolo[1,2-c][1,2,4]triazolo[1,5-a]pyrimidin-6-yl)piperazine-1-carboxylate (2.35 g, 88% pure, yield: 10%).

LC-MS: Rt=1.34 min; MS m/z [M+H−Boc]⁺ 574.1/576.1/578.1, m/z [M−H]⁻ 672.4/674.3/676.4; UPLC-MS 1

LC-MS: Rt=6.62 min; MS m/z [M+H−Boc]⁺ 574.1/576.1/578.1, m/z [M−H]⁻ 672.4/674.3/676.4; UPLC-MS 2

Intermediate F2: All pure fractions containing peak 2 were combined, concentrated under reduced pressure and dried under HV to give the title compound as a racemic mixture of and tert-butyl 4-((7S,9S)-2-bromo-9-((2-chloro-4-(trifluoromethyl)phenyl)carbamoyl)-7-methyl-5-oxo-5,7,8,9-tetrahydropyrrolo[1,2-c][1,2,4]triazolo[1,5-a]pyrimidin-6-yl)piperazine-1-carboxylate and tert-butyl 4-((7R,9R)-2-bromo-9-((2-chloro-4-(trifluoromethyl)phenyl)carbamoyl)-7-methyl-5-oxo-5,7,8,9-tetrahydropyrrolo[1,2-c][1,2,4]triazolo[1,5-a]pyrimidin-6-yl)piperazine-1-carboxylate (4.47 g, 51% pure, yield: 11%).

Total: (9.27 g, 72% pure, yield: 33%)

LC-MS: Rt=1.38 min; MS m/z [M+H−Boc]⁺ 574.2/576.2/578.2, m/z [M−H]⁻ 672.2/674.1/676.2; UPLC-MS 1

LC-MS: Rt=6.71 min; MS m/z [M+H−Boc]⁺574.1/576.1/578.1, m/z [M−H]⁻ 672.4/674.4/676.3; UPLC-MS 2

### Intermediate G1: tert-butyl 4-((7R,9S)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate and Intermediate G2: tert-butyl 4-((7S,9R)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate

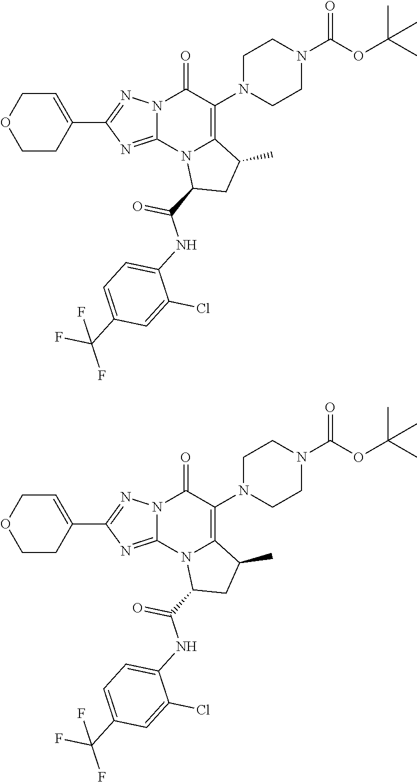 [SMILES: C[C@@H]1C[C@@H](C(=O)Nc2ccc(C(F)(F)F)cc2Cl)n2c1c(N1CCN(C(=O)OC(C)(C)C)CC1)c(=O)n1nc(C3=CCOCC3)nc21 ; C[C@H]1C[C@H](C(=O)Nc2ccc(C(F)(F)F)cc2Cl)n2c1c(N1CCN(C(=O)OC(C)(C)C)CC1)c(=O)n1nc(C3=CCOCC3)nc21]

To a stirred solution of a racemic mixture of tert-butyl 4-((7R,9S)-2-bromo-9-((2-chloro-4-(trifluoromethyl)phenyl)carbamoyl)-7-methyl-5-oxo-5,7,8,9-tetrahydropyrrolo[1,2-c][1,2,4]triazolo[1,5-a]pyrimidin-6-yl)piperazine-1-carboxylate and tert-butyl 4-((7S,9R)-2-bromo-9-((2-chloro-4-(trifluoromethyl)phenyl)carbamoyl)-7-methyl-5-oxo-5,7,8,9-tetrahydropyrrolo[1,2-c][1,2,4]triazolo[1,5-a]pyrimidin-6-yl)piperazine-1-carboxylate (Intermediate F1) (2.35 g, 3.06 mmol), K₃PO₄ (1.95 g, 9.19 mmol) and PdCl₂(dppf)·DCM adduct (125 mg, 153 μmol) in 1,4-dioxane (40 mL) and water (20 mL) was added 2-(3,6-dihydro-2H-pyran-4-yl)-4,4,5,5-tetramethyl-1,3,2-dioxaborolane (837 mg, 3.98 mmol) and the RM was stirred at 85° C. for 3 days. The RM was diluted with EtOAc and water, extracted twice with EtOAc and the combined organic layers were washed with brine, dried over Na₂SO₄ and concentrated under reduced pressure. The crude product was purified twice by column chromatography (RediSep Column: Silica 80 g, eluent heptane:EtOAc 50:50 to 0:100) and (RediSep Column: Silica 80 g gold, eluent DCM:EtOAc 100:0 to 0:100). All fractions containing peak 1 were combined, concentrated under reduced pressure and dried under HV to give the title compound as a racemic mixture (722 mg, 77% pure, yield: 27%).

LC-MS: Rt=1.32 min; MS m/z [M+H−Boc]⁺ 578.4/580.3, m/z [M−H]⁻ 676.5/678.5; UPLC-MS 1

LC-MS: Rt=6.45 min; MS m/z [M+H−Boc]⁺ 578.3/580.3, m/z [M−H]⁻ 676.5/678.4; UPLC-MS 2

The racemate (1.20 g) was purified by preparative chiral SFC (instrument: Sepiatec Prep SFC 100; column: OVEN3 Chiralpak IB-N 250 mm×30 mm 5 μm; eluent: A: 25% IPA+0.1% NH₃, B: 75% scCO₂; flow rate: 80.0 mL/min; detection: UV; injection volume: 1.0 mL; gradient: isocratic A: 25%, B: 75%; oven temperature: 40° C.; BPR: 110 bar).

Peak 1: tert-butyl 4-((7R,9S)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate (Intermediate G1) 499 mg, 97% pure, yield: 43%

LC-MS: Rt=1.30 min; MS m/z [M+H−Boc]⁺578.2/580.3, m/z [M−H]⁻ 676.3/678.3; UPLC-MS 1

LC-MS: Rt=6.47 min; MS m/z [M+H−Boc]⁺ 578.2/580.2, m/z [M−H]⁻ 676.3/678.2; UPLC-MS 2

Chiral HPLC (C-HPLC 9): Rt=1.21 min, 99.5% ee

Peak 2: tert-butyl 4-((7S,9R)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate (Intermediate G2)

532 mg, 98% pure, yield: 46%

LC-MS: Rt=1.30 min; MS m/z [M+H−Boc]⁺ 578.3/580.3, m/z [M−H]⁻ 676.3/678.3; UPLC-MS 1

LC-MS: Rt=6.46 min; MS m/z [M+H−Boc]⁺ 578.2/580.2, m/z [M−H]⁻ 676.3/678.2; UPLC-MS 2

Chiral HPLC (C-HPLC 9): Rt=2.03 min, 99.5% ee

### Intermediate G3: tert-butyl 4-((7S,9S)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate and Intermediate G4: tert-butyl 4-((7R,9R)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate

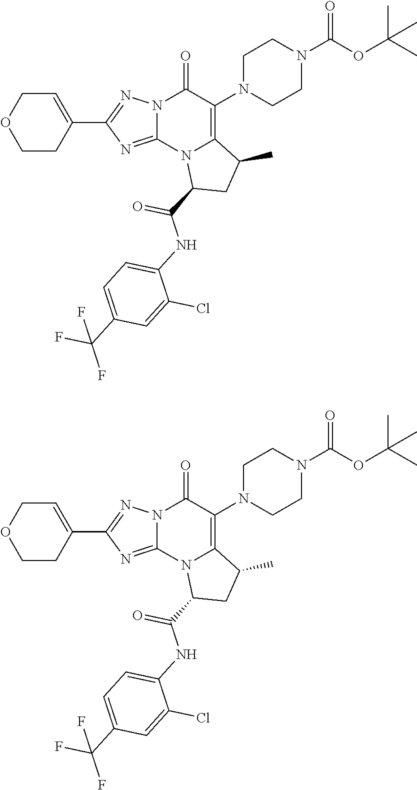 [SMILES: C[C@H]1C[C@@H](C(=O)Nc2ccc(C(F)(F)F)cc2Cl)n2c1c(N1CCN(C(=O)OC(C)(C)C)CC1)c(=O)n1nc(C3=CCOCC3)nc21 ; C[C@@H]1C[C@H](C(=O)Nc2ccc(C(F)(F)F)cc2Cl)n2c1c(N1CCN(C(=O)OC(C)(C)C)CC1)c(=O)n1nc(C3=CCOCC3)nc21]

To a stirred solution of the racemic mixture of tert-butyl 4-((7S,9S)-2-bromo-9-((2-chloro-4-(trifluoromethyl)phenyl)carbamoyl)-7-methyl-5-oxo-5,7,8,9-tetrahydropyrrolo[1,2-c][1,2,4]triazolo[1,5-a]pyrimidin-6-yl)piperazine-1-carboxylate and tert-butyl 4-((7R,9R)-2-bromo-9-((2-chloro-4-(trifluoromethyl)phenyl)carbamoyl)-7-methyl-5-oxo-5,7,8,9-tetrahydropyrrolo[1,2-c][1,2,4]triazolo[1,5-a]pyrimidin-6-yl)piperazine-1-carboxylate (Intermediate F2) (4.47 g, 3.97 mmol), K₃PO₄ (2.53 g, 11.9 mmol) and PdCl₂(dppf)·DCM adduct (162 mg, 199 μmol) in 1,4-dioxane (40 mL) and water (20 mL) was added at RT 2-(3,6-dihydro-2H-pyran-4-yl)-4,4,5,5-tetramethyl-1,3,2-dioxaborolane (1.09 g, 5.17 mmol) and the RM was stirred at 80° C. for 1 hour. The RM was diluted with EtOAc and water, extracted twice with EtOAc and the combined organic layers were washed with brine, dried over Na₂SO₄ and concentrated under reduced pressure. The crude product was purified twice by column chromatography (RediSep Column: Silica 80 g, eluent DCM:EtOAc 100:0 to 20:80), (RediSep Column: Silica 80 g, eluent DCM:EtOAc 100:0 to 20:80). The pure product containing fractions were combined, concentrated under reduced pressure and dried under HV to give the title compound (1.66 g, 96% pure, yield: 59%). The impure product containing fractions were combined, concentrated under reduced pressure and dried under HV to give the title compound (1.20 g, 64% pure, yield: 29%).

LC-MS: Rt=1.32 min; MS m/z [M+H−Boc]⁺ 578.3/580.3, m/z [M−H]⁻ 676.5/678.5; UPLC-MS 1

LC-MS: Rt=6.51 min; MS m/z [M+H−Boc]⁺ 578.3/580.3, m/z [M−H]⁻ 676.5/678.5; UPLC-MS 2

The racemate (1.66 g) was purified by preparative chiral SFC (instrument: Sepiatec Prep SFC100; column: OVEN3 Chiralpak IB-N 250 mm×30 mm 5 μm; eluent: A: 40% MeOH+0.1% NH₃, B: 60% scCO₂; flow rate: 90.0 mL/min; detection: UV; injection volume: 1.8 mL; gradient: isocratic A: 40%, B: 60%; BPR: 130 bar).

Peak 1: tert-butyl 4-((7S,9S)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate (Intermediate G3)

822 mg, 91% pure, yield: 47%

LC-MS: Rt=1.33 min; MS m/z [M+H]⁺ 678.3/680.4, m/z [M−H]⁻ 676.3/678.2; UPLC-MS 1

Chiral HPLC (C-HPLC 8): Rt=0.67 min, 99% ee

Peak 2: tert-butyl 4-((7R,9R)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate (Intermediate G4)

712 mg, 100% pure, yield: 45%

LC-MS: Rt=1.33 min; MS m/z [M+H]⁺ 678.3/680.3, m/z [M−H]⁻ 676.2/678.3; UPLC-MS 1

Chiral HPLC (C-HPLC 8): Rt=1.87 min, 99.5% ee

### Intermediate H1: (7R,9S)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide

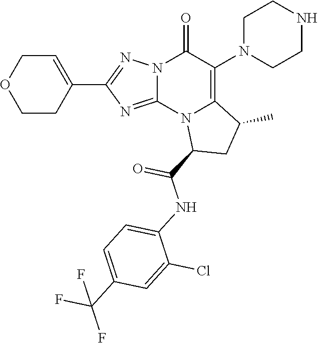 [SMILES: C[C@@H]1C[C@@H](C(=O)Nc2ccc(C(F)(F)F)cc2Cl)n2c1c(N1CCNCC1)c(=O)n1nc(C3=CCOCC3)nc21]

4N HCl in 1,4-dioxane (5.00 mL, 20.0 mmol) was added at RT to tert-butyl 4-((7R,9S)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate (Intermediate G1) (500 mg, 737 μmol) and the RM was stirred at RT for 1 hour. MeOH and DCM were added to the suspension, which turned into a solution. This solution was treated with aq NaHCO₃ and the aqueous layer was extracted 3 times with 10% MeOH in DCM and the combined organic layers were dried over Na₂SO₄ and concentrated under reduced pressure to give the title compound as a yellow solid (466 mg, 90% pure, yield: 98%).

LC-MS: Rt=0.77 min; MS m/z [M+H]⁺ 578.5/580.5, m/z [M−H]⁻ 576.1/578.1; UPLC-MS 1

### Intermediate H2: (7S,9R)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide

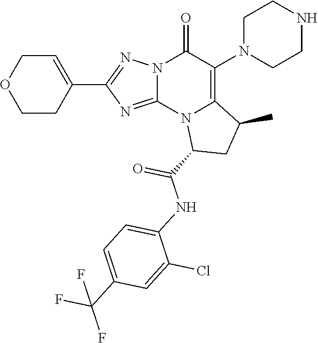 [SMILES: C[C@H]1C[C@H](C(=O)Nc2ccc(C(F)(F)F)cc2Cl)n2c1c(N1CCNCC1)c(=O)n1nc(C3=CCOCC3)nc21]

4N HCl in 1,4-dioxane (5.00 mL, 20.0 mmol) was added at RT to tert-butyl 4-((7S,9R)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate (Intermediate G2) (530 mg, 782 μmol) and the RM was stirred at RT for 1 hour. MeOH and DCM were added to the suspension, which turned into a solution. This solution was treated with aq NaHCO₃ and the aqueous layer was extracted 3 times with 10% MeOH in DCM and the combined organic layers were dried over Na₂SO₄ and concentrated under reduced pressure to give the title compound as a yellow solid (497 mg, 90% pure, yield: 99%).

LC-MS: Rt=0.77 min; MS m/z [M+H]⁺ 578.5/580.5, m/z [M−H]⁻ 576.2/578.1; UPLC-MS 1

### Intermediate H3: (7S,9S)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide

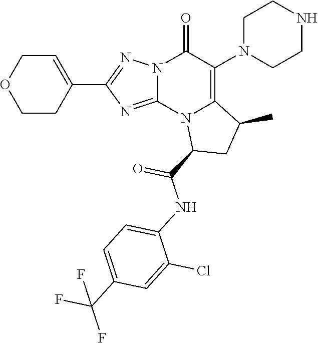 [SMILES: C[C@H]1C[C@@H](C(=O)Nc2ccc(C(F)(F)F)cc2Cl)n2c1c(N1CCNCC1)c(=O)n1nc(C3=CCOCC3)nc21]

Tert-butyl 4-((7S,9S)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate (Intermediate G3) (822 mg, 1.10 mmol) in 4N HCl in 1,4-dioxane (5.00 mL, 20.0 mmol) was stirred at RT for 20 minutes. The RM was concentrated then the residue was diluted with DCM and aq NaHCO₃, extracted twice with DCM and the combined organic layers were dried over Na₂SO₄ and concentrated under reduced pressure to give a white foam. The crude product was purified by column chromatography (RediSep Column: Silica 24 g, eluent DCM:7N NH₃ in MeOH 100:0 to 85:15). The product containing fractions were combined, concentrated under reduced pressure and dried under HV to give the title compound (603 mg, 98% pure, yield: 93%).

LC-MS: Rt=0.76 min; MS m/z [M+H]⁺ 578.4/580.4, m/z [M−H]⁻ 576.1/578.1; UPLC-MS 1

LC-MS: Rt=4.00 min; MS m/z [M+H]⁺ 578.4/580.4, m/z [M−H]⁻ 576.3/578.1; UPLC-MS 2

### Intermediate H4: (7R,9R)—N-(2-chloro-4-(trifluoromethyl)phenyl)-2-(3,6-dihydro-2H-pyran-4-yl)-7-methyl-5-oxo-6-(piperazin-1-yl)-5,7,8,9-tetrahydropyrrolo[1,2-c][1,2,4]triazolo[1,5-a]pyrimidine-9-carboxamide

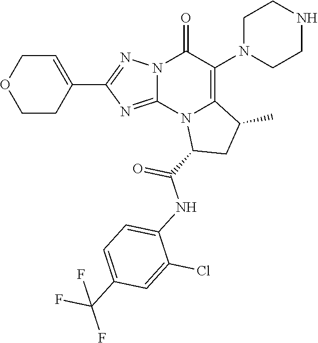 [SMILES: C[C@@H]1C[C@H](C(=O)Nc2ccc(C(F)(F)F)cc2Cl)n2c1c(N1CCNCC1)c(=O)n1nc(C3=CCOCC3)nc21]

Tert-butyl 4-((7R,9R)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)piperazine-1-carboxylate (Intermediate G4) (712 mg, 1.05 mmol) in 4N HCl in 1,4-dioxane (5.00 mL, 20.0 mmol) was stirred at RT for 1 hour. The RM was diluted with DCM and aq NaHCO₃, extracted 3 times with 10% MeOH in DCM and the combined organic layers were dried over Na₂SO₄ and concentrated under reduced pressure to give the title compound as a beige foam (607 mg, 100% pure, yield: 100%).

LC-MS: Rt=0.78 min; MS m/z [M+H]⁺ 578.4/580.4, m/z [M−H]⁻ 576.3/578.2; UPLC-MS 1

### Intermediate I1: tert-butyl 4-((7S,9R)-2-bromo-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate and tert-butyl 4-((7R,9S)-2-bromo-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate and Intermediate I2: tert-butyl 4-((7S,9S)-2-bromo-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate and tert-butyl 4-((7R,9R)-2-bromo-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate

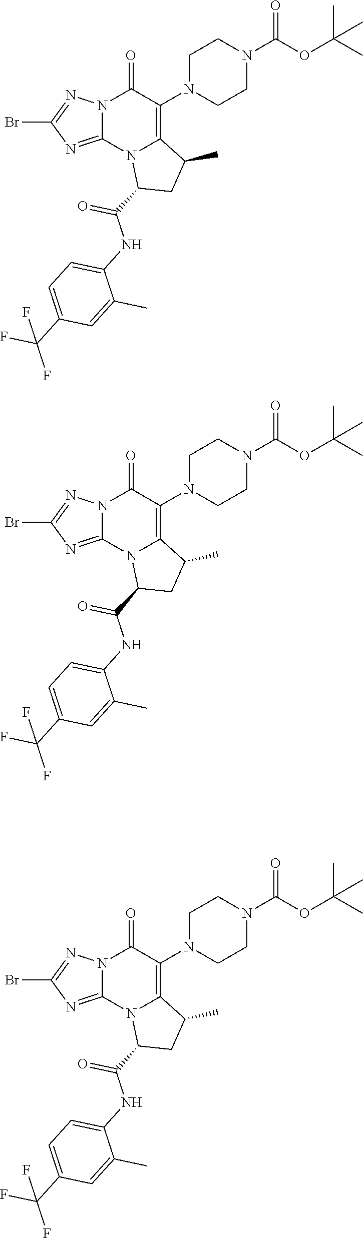 [SMILES: Cc1cc(C(F)(F)F)ccc1NC(=O)[C@H]1C[C@H](C)c2c(N3CCN(C(=O)OC(C)(C)C)CC3)c(=O)n3nc(Br)nc3n21 ; Cc1cc(C(F)(F)F)ccc1NC(=O)[C@@H]1C[C@@H](C)c2c(N3CCN(C(=O)OC(C)(C)C)CC3)c(=O)n3nc(Br)nc3n21 ; Cc1cc(C(F)(F)F)ccc1NC(=O)[C@H]1C[C@@H](C)c2c(N3CCN(C(=O)OC(C)(C)C)CC3)c(=O)n3nc(Br)nc3n21]

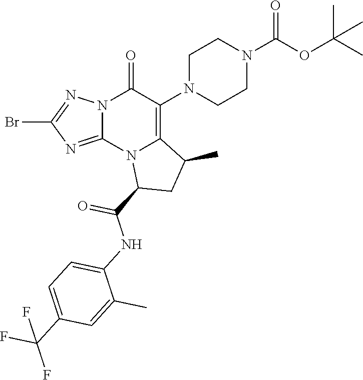 [SMILES: Cc1cc(C(F)(F)F)ccc1NC(=O)[C@@H]1C[C@H](C)c2c(N3CCN(C(=O)OC(C)(C)C)CC3)c(=O)n3nc(Br)nc3n21]

A pale yellow solution of 2-bromo-6-(4-(tert-butoxycarbonyl)piperazin-1-yl)-7-methyl-5-oxo-5,7,8,9-tetrahydropyrrolo[1,2-c][1,2,4]triazolo[1,5-a]pyrimidine-9-carboxylic acid (Intermediate B) (189 mg, 372 μmol) and 2-methyl-4-(trifluoromethyl)aniline (71.8 mg, 410 μmol) in DCM (10 mL) was cooled to −10° C. Then Et₃N (413 μL, 2.98 mmol) was added, followed by dropwise addition of T₃P 50% solution (443 μL, 745 μmol). The RM was stirred at RT for 1 day. T₃P 50% solution (443 μL, 745 μmol) was added and the RM was stirred at RT for 1 day. The RM was diluted with DCM (10 mL) and extracted with aq sat NaHCO₃ (2×20 mL) and brine (20 mL). The aqueous layer was washed with DCM (20 mL). The combined organic layers were dried over anhydrous Na₂SO₄, filtered and concentrated under reduced pressure at 45° C. to give a pale brown resin (288 mg). The crude product was purified by column chromatography (RediSep Column: Silica 40 g, eluent n-heptane:EtOAc 80:20 to 0:100).

Intermediate I1: All fractions containing peak 1 were combined, concentrated under reduced pressure and dried under HV to give the title compound as a racemic mixture of tert-butyl 4-((7S,9R)-2-bromo-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate and tert-butyl 4-((7R,9S)-2-bromo-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate as a beige solid (108 mg, 92% pure, yield: 41%).

LC-MS: Rt=6.33 min; MS m/z [M+H−Boc]⁺ 554.5/556.5, m/z [M−H]⁻ 652.4/654.4; UPLC-MS 2

Intermediate I2: All fractions containing peak 2 were combined, concentrated under reduced pressure and dried under HV to give the title compound as a racemic mixture of tert-butyl 4-((7S,9S)-2-bromo-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate and tert-butyl 4-((7R,9R)-2-bromo-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate as a beige solid (88.2 mg, 97% pure, yield: 35%).

LC-MS: Rt=6.41 min; MS m/z [M+H−Boc]⁺ 554.4/556.4, m/z [M−H]⁻ 652.4/654.4; UPLC-MS 2

### Intermediate J1: tert-butyl 4-((7S,9R)-2-(3,6-dihydro-2H-pyran-4-yl)-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate and Intermediate J2: tert-butyl 4-((7R,9S)-2-(3,6-dihydro-2H-pyran-4-yl)-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate

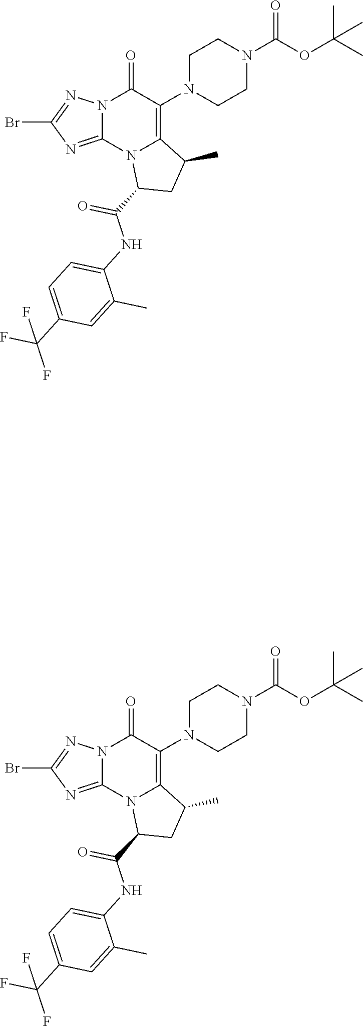 [SMILES: Cc1cc(C(F)(F)F)ccc1NC(=O)[C@H]1C[C@H](C)c2c(N3CCN(C(=O)OC(C)(C)C)CC3)c(=O)n3nc(Br)nc3n21 ; Cc1cc(C(F)(F)F)ccc1NC(=O)[C@@H]1C[C@@H](C)c2c(N3CCN(C(=O)OC(C)(C)C)CC3)c(=O)n3nc(Br)nc3n21]

The racemic mixture of tert-butyl 4-((7S,9R)-2-bromo-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate and tert-butyl 4-((7R,9S)-2-bromo-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate (Intermediate I1) (105 mg, 160 μmol), 2-(3,6-dihydro-2H-pyran-4-yl)-4,4,5,5-tetramethyl-1,3,2-dioxaborolane (47.2 mg, 225 μmol) and XPhos Pd G3 (6.79 mg, 8.02 μmol) were dissolved in 1.4-dioxane (12 mL). Then K₃PO₄ 1M in water (481 μL, 481 μmol) was added. The RM was stirred at 80° C. for 30 minutes. The RM was extracted with water (15 mL) and TBME (15 mL). The organic layer was washed with water (15 mL) and brine (15 mL). The aqueous layer was washed with TBME (2×15 mL). The combined organic layers were dried over anhydrous Na₂SO₄, filtered and concentrated under reduced pressure at 45° C. to give a yellow solid (128 mg). The crude product was dissolved in 10% MeOH in DCM and SiliaMetS®Thiol was added. The mixture was stirred at 40° C. for 1 hour, then it was filtered. The cake was washed with DCM. The filtrate was concentrated under reduced pressure at 45° C. to give a yellow solid (121 mg). The crude product was purified by column chromatography (RediSep Column: Silica 24 g, eluent n-heptane:EtOAc 50:50 to 0:100). The product containing fractions were combined and concentrated under reduced pressure to give a yellow foam (98.1 mg).

The yellow foam (76.0 mg) was purified by preparative chiral HPLC (instrument: Waters Prep SFC100 MS; column: Chiralpak IC 250 mm×30 mm 5 μm; eluent: A: 40% MeOH+0.1% NH₃, B: 60% scCO₂; flow rate: 80.0 mL/min; detection: DAD; injection volume: 3.0 mL; gradient: isocratic A: 40%, B: 60%).

Peak 1: tert-butyl 4-((7S,9R)-2-(3,6-dihydro-2H-pyran-4-yl)-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate (Intermediate J1)

pale yellow solid (25.5 mg, 99% pure, yield: 24%)

LC-MS: Rt=1.28 min; m/z [M−H]⁻ 656.5; UPLC-MS 1

Chiral HPLC (C-HPLC 7): Rt=1.13 min, 99.5% ee

Peak 2: tert-butyl 4-((7R,9S)-2-(3,6-dihydro-2H-pyran-4-yl)-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate (Intermediate J2)

pale yellow solid (25.3 mg, 99% pure, yield: 24%)

LC-MS: Rt=1.28 min; MS m/z [M+H]⁺ 658.4, m/z [M−H]⁻ 656.5; UPLC-MS 1

Chiral HPLC (C-HPLC 7): Rt=1.99 min, 99.5% ee

### Intermediate J3: tert-butyl 4-((7S,9S)-2-(3,6-dihydro-2H-pyran-4-yl)-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate and Intermediate J4: tert-butyl 4-((7R,9R)-2-(3,6-dihydro-2H-pyran-4-yl)-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate

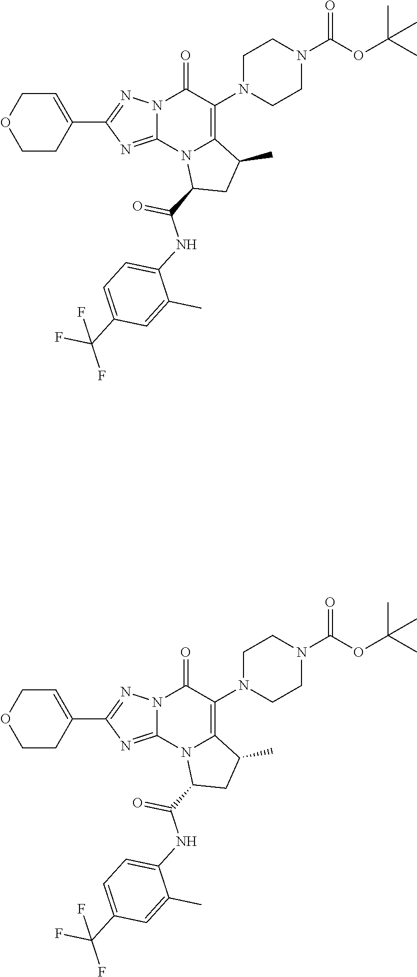 [SMILES: Cc1cc(C(F)(F)F)ccc1NC(=O)[C@@H]1C[C@H](C)c2c(N3CCN(C(=O)OC(C)(C)C)CC3)c(=O)n3nc(C4=CCOCC4)nc3n21 ; Cc1cc(C(F)(F)F)ccc1NC(=O)[C@H]1C[C@@H](C)c2c(N3CCN(C(=O)OC(C)(C)C)CC3)c(=O)n3nc(C4=CCOCC4)nc3n21]

The racemic mixture of tert-butyl 4-((7S,9S)-2-bromo-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate and tert-butyl 4-((7R,9R)-2-bromo-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate (Intermediate I2) (86.0 mg, 131 μmol), 2-(3,6-dihydro-2H-pyran-4-yl)-4,4,5,5-tetramethyl-1,3,2-dioxaborolane (33.1 mg, 158 μmol) and XPhos Pd G3 (5.56 mg, 6.57 μmol) were dissolved in 1.4-dioxane (2.5 mL). Then K₃PO₄ 1 M in water (394 μL, 394 μmol) was added. The RM was stirred at 80° C. for 30 minutes. The RM was extracted with water (15 mL) and TBME (15 mL). The organic layer was washed with water (15 mL) and brine (15 mL). The aqueous layer was washed with TBME (2×15 mL). The combined organic layers were dried over anhydrous Na₂SO₄, filtered and concentrated under reduced pressure at 45° C. to give a yellow solid (110 mg). The crude product was dissolved in 10% MeOH in DCM and SiliaMetS®Thiol (140 mg) was added. The mixture was stirred at 40° C. for 1 hour, then it was filtered. The cake was washed with DCM. The filtrate was concentrated under reduced pressure at 45° C. to give a yellow solid (106 mg). The crude product was purified by column chromatography (RediSep Column: Silica 24 g, eluent n-heptane:EtOAc 40:60 to 0:100). The product containing fractions were combined and concentrated under reduced pressure to give a yellow foam (76.1 mg).

The yellow foam (76.0 mg) was purified by preparative chiral HPLC (instrument: Waters Prep SFC100 MS; column: Chiralpak IB-N 250 mm×30 mm 5 μm; eluent: A: 40% MeOH+0.1% NH₃, B: 60% scCO₂; flow rate: 80.0 mL/min; detection: DAD; injection volume: 4.0 mL; gradient: isocratic A: 40%, B: 60%; oven temperature: 40° C.; BPR: 120 bar).

Peak 1: tert-butyl 4-((7S,9S)-2-(3,6-dihydro-2H-pyran-4-yl)-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate (Intermediate J3)

pale yellow solid (33.3 mg, 83% pure, yield: 32%)

LC-MS: Rt=1.30 min; MS m/z [M+H−Boc]⁺ 558.6, m/z [M−H]⁻ 656.6; UPLC-MS 1

Chiral HPLC (C-HPLC 10): Rt=0.65 min, 99.5% ee

Peak 2: tert-butyl 4-((7R,9R)-2-(3,6-dihydro-2H-pyran-4-yl)-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate (Intermediate J4)

pale yellow solid (24.3 mg, 98% pure, yield: 27.5%)

LC-MS: Rt=1.30 min; MS m/z [M+H−Boc]⁺ 558.6, m/z [M−H]⁻ 656.6; UPLC-MS 1

Chiral HPLC (C-HPLC 10): Rt=2.19 min, 99.5% ee

### Intermediate K1: (7S,9R)-2-(3,6-dihydro-2H-pyran-4-yl)-7-methyl-N-(2-methyl-4-(trifluoromethyl)phenyl)-5-oxo-6-(piperazin-1-yl)-5,7,8,9-tetrahydropyrrolo[1,2-c][1,2,4]triazolo[1,5-a]pyrimidine-9-carboxamide

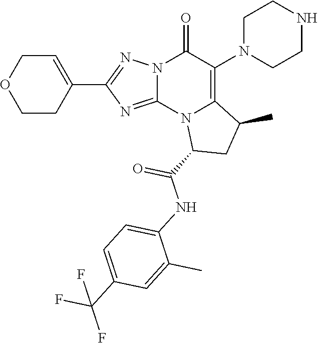 [SMILES: Cc1cc(C(F)(F)F)ccc1NC(=O)[C@H]1C[C@H](C)c2c(N3CCNCC3)c(=O)n3nc(C4=CCOCC4)nc3n21]

To a solution of tert-butyl 4-((7S,9R)-2-(3,6-dihydro-2H-pyran-4-yl)-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate (Intermediate J1) (24.0 mg, 36.0 μmol) in 1,4-dioxane (500 μL) was added 4M HCl in 1,4-dioxane (639 μL, 2.55 mmol) and the RM was stirred at RT for 18 hours. The RM was concentrated under reduced pressure at 45° C. to give the title compound dihydrochlorid salt as a yellow solid (25.5 mg, 95%, quantitative).

LC-MS: Rt=0.78 min; MS m/z [M+H]⁺ 558.6, m/z [M−H]⁻ 556.5; UPLC-MS 1

### Intermediate K2: (7R,9S)-2-(3,6-dihydro-2H-pyran-4-yl)-7-methyl-N-(2-methyl-4-(trifluoromethyl)phenyl)-5-oxo-6-(piperazin-1-yl)-5,7,8,9-tetrahydropyrrolo[1,2-c][1,2,4]triazolo[1,5-a]pyrimidine-9-carboxamide

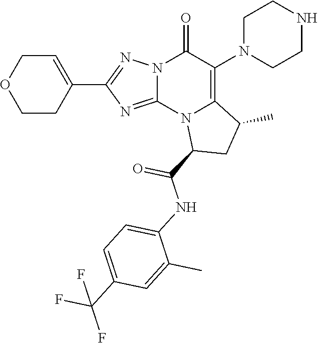 [SMILES: Cc1cc(C(F)(F)F)ccc1NC(=O)[C@@H]1C[C@@H](C)c2c(N3CCNCC3)c(=O)n3nc(C4=CCOCC4)nc3n21]

To a solution of tert-butyl 4-((7R,9S)-2-(3,6-dihydro-2H-pyran-4-yl)-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate (Intermediate J2) (24.0 mg, 36.0 μmol) in 1,4-dioxane (500 μL) was added 4M HCl in 1,4-dioxane (639 μL, 2.55 mmol) and the RM was stirred at RT for 18 hours. The RM was concentrated under reduced pressure at 45° C. to give the title compound dihydrochlorid salt as a pale yellow solid (25.4 mg, 95%, quantitative).

LC-MS: Rt=0.77 min; MS m/z [M+H]⁺ 558.6, m/z [M−H]⁻ 556.5; UPLC-MS 1

### Intermediate K3: (7S,9S)-2-(3,6-dihydro-2H-pyran-4-yl)-7-methyl-N-(2-methyl-4-(trifluoromethyl)phenyl)-5-oxo-6-(piperazin-1-yl)-5,7,8,9-tetrahydropyrrolo[1,2-c][1,2,4]triazolo[1,5-a]pyrimidine-9-carboxamide

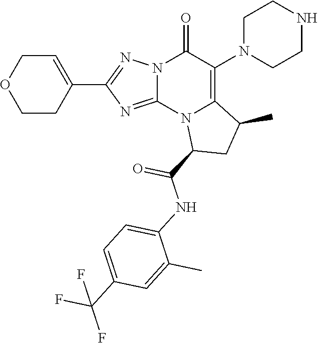 [SMILES: Cc1cc(C(F)(F)F)ccc1NC(=O)[C@@H]1C[C@H](C)c2c(N3CCNCC3)c(=O)n3nc(C4=CCOCC4)nc3n21]

To a solution of tert-butyl 4-((7S,9S)-2-(3,6-dihydro-2H-pyran-4-yl)-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate (Intermediate J3) (19.3 mg, 29.0 μmol) in 1,4-dioxane (500 μL) was added 4M HCl in 1,4-dioxane (514 μL, 2.05 mmol) and the RM was stirred at RT for 17 hours. The RM was concentrated under reduced pressure at 45° C. to give the title compound dihydrochlorid salt as a yellow solid (23.0 mg, 95%, quantitative).

LC-MS: Rt=0.75 min; MS m/z [M+H]⁺ 558.3, m/z [M−H]⁻ 556.4; UPLC-MS 1

### Intermediate K4: (7R,9R)-2-(3,6-dihydro-2H-pyran-4-yl)-7-methyl-N-(2-methyl-4-(trifluoromethyl)phenyl)-5-oxo-6-(piperazin-1-yl)-5,7,8,9-tetrahydropyrrolo[1,2-c][1,2,4]triazolo[1,5-a]pyrimidine-9-carboxamide

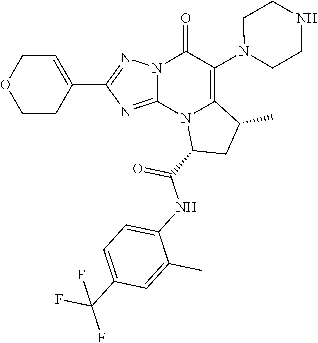 [SMILES: Cc1cc(C(F)(F)F)ccc1NC(=O)[C@H]1C[C@@H](C)c2c(N3CCNCC3)c(=O)n3nc(C4=CCOCC4)nc3n21]

To a solution of tert-butyl 4-((7R,9R)-2-(3,6-dihydro-2H-pyran-4-yl)-7-methyl-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate (Intermediate J4) (23.3 mg, 35.0 μmol) in 1,4-dioxane (500 μL) was added 4M HCl in 1,4-dioxane (531 μL, 2.13 mmol) and the RM was stirred at RT for 17 hours. The RM was concentrated under reduced pressure at 45° C. to give the title compound dihydrochlorid salt as a pale yellow solid (25.4 mg, 97%, quantitative).

LC-MS: Rt=0.76 min; MS m/z [M+H]⁺ 558.3, m/z [M−H]⁻ 556.4; UPLC-MS 1

### Intermediate L: 2-bromo-6-((R)-4-(tert-butoxycarbonyl)-3-methylpiperazin-1-yl)-7-methyl-5-oxo-5,7,8,9-tetrahydropyrrolo[1,2-c][1,2,4]triazolo[1,5-a]pyrimidine-9-carboxylic acid

### Step 1: tert-butyl (R)-4-(2-bromo-5-ethyl-7-oxo-4,7-dihydro-[1,2,4]triazolo[1,5-a]pyrimidin-6-yl)-2-methylpiperazine-1-carboxylate

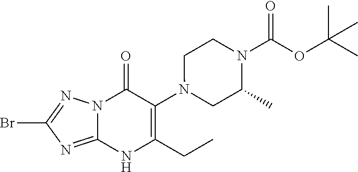 [SMILES: CCc1[nH]c2nc(Br)nn2c(=O)c1N1CCN(C(=O)OC(C)(C)C)[C@H](C)C1]

Tert-butyl (2R)-4-(1-ethoxy-1,3-dioxopentan-2-yl)-2-methylpiperazine-1-carboxylate (Intermediate AE) (16.8 g, 48.1 mmol) was dissolved in EtOH (45 mL) and 3-bromo-1H-1,2,4-triazol-5-amine (Intermediate AI) (7.50 g, 43.7 mmol) was added, followed by H₃PO₄ (5.29 g, 45.9 mmol). The RM was stirred at 90° C. for 44 hours. DIPEA (22.9 mL, 131 mmol) and Boc₂O (5.08 mL, 21.9 mmol) were added. The RM was stirred at RT for 1.5 hours. The reaction was quenched with water, diluted with EtOAc and concentrated under reduced pressure. The residue was extracted with EtOAc (3×500 mL) and the organic layers were washed with water (2×50 mL) and brine (2×50 mL), dried through a phase separator and concentrated under reduced pressure. This material was suspended in Et₂O (1 L) and stirred at reflux for 20 minutes. The suspension was filtered, and the mother liquor was concentrated under reduced pressure. This material was mixed with hexane (2×600 mL), stirred at 40° C., and then filtered. The cake was dissolved in DCM and MeOH, concentrated again and adsorbed onto Isolute and purified twice by column chromatography (RediSep Column: Silica 120 g, eluent DCM:MeOH 100:0 to 85:15) and (RediSep Column: Silica 80 g, eluent DCM:MeOH 100:0 to 85:15). The product containing fractions were combined and concentrated under reduced pressure to give the title compound as a beige solid 6.61 g, 99% pure, yield: 34%).

LC-MS: Rt=0.94 min; MS m/z [M+H−Boc]⁺ 341.1, m/z [M−H]⁻ 439.3; UPLC-MS 3

¹H NMR (400 MHz, DMSO-d₆) δ 13.27 (s, 1H), 4.19 (m, 1H), 3.76 (m, 1H), 3.52 (m, 1H), 3.37 (m, 1H), 3.10 (m, 1H), 2.86 (m, 1H), 2.71 (m, 3H), 1.44 (s, 9H), 1.26 (m, 3H), 1.21 (t, J=7.5 Hz, 3H)

### Step 2: tert-butyl (R)-4-(2-bromo-5-ethyl-7-oxo-4-((2-(trimethylsilyl)ethoxy)methyl)-4,7-dihydro-[1,2,4]triazolo[1,5-a]pyrimidin-6-yl)-2-methylpiperazine-1-carboxylate

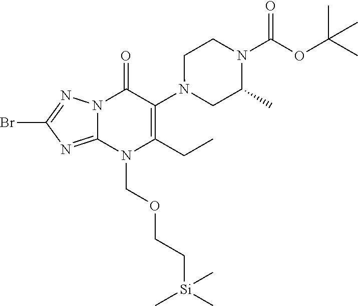 [SMILES: CCc1c(N2CCN(C(=O)OC(C)(C)C)[C@H](C)C2)c(=O)n2nc(Br)nc2n1COCC[Si](C)(C)C]

To tert-butyl (R)-4-(2-bromo-5-ethyl-7-oxo-4,7-dihydro-[1,2,4]triazolo[1,5-a]pyrimidin-6-yl)-2-methylpiperazine-1-carboxylate (3.23 g, 7.32 mmol) in THF (60 mL) and DMF (10 mL) under nitrogen at −20° C. was added portionwise NaH (263 mg, 11.0 mmol). The RM was stirred at −20° C. for 35 minutes. Then (2-(chloromethoxy)ethyl)trimethylsilane (2.60 mL, 14.7 mmol) was added dropwise. The RM was stirred at −20° C. for 1 hour. Then continued stirring at −20° C. to −10° C. for 2 hours. Aq sat NaHCO₃ (30 mL) and TBME (30 mL) were added slowly. The cooling bath was removed and the RM was stirred for 10 minutes. The RM was further diluted with TBME (50 mL) and the organic layer was separated. The aqueous layer was extracted with TBME (30 mL). The crude product was purified by column chromatography (RediSep Column: Silica 80 g, eluent n-heptane:EtOAc 95:5 to 20:80). The product containing fractions were combined, concentrated under reduced pressure, triturated with heptane, filtered and dried to give the title compound as a colourless solid (2.89 g, 100% pure, yield: 69%).

LC-MS: Rt=7.50 min; MS m/z [M+H−Boc]⁺471.1/473.1; UPLC-MS 2

### Step 3: tert-butyl (2R)-4-(2-bromo-7-oxo-5-(pent-4-en-2-yl)-4-((2-(trimethylsilyl)ethoxy)methyl)-4,7-dihydro-[1,2,4]triazolo[1,5-a]pyrimidin-6-yl)-2-methylpiperazine-1-carboxylate

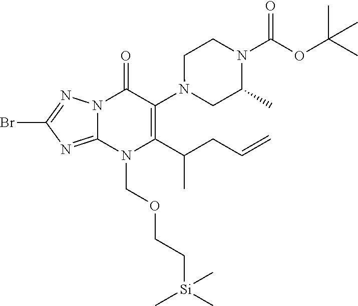 [SMILES: C=CCC(C)c1c(N2CCN(C(=O)OC(C)(C)C)[C@H](C)C2)c(=O)n2nc(Br)nc2n1COCC[Si](C)(C)C]

To tert-butyl (R)-4-(2-bromo-5-ethyl-7-oxo-4-((2-(trimethylsilyl)ethoxy)methyl)-4,7-dihydro-[1,2,4]triazolo[1,5-a]pyrimidin-6-yl)-2-methylpiperazine-1-carboxylate (2.87 g, 5.02 mmol) in THF (80 mL) at −78° C. was added dropwise over 10 minutes NaHMDS 1M in THF (6.03 mL, 6.03 mmol). The resulting yellow solution was stirred at −78° C. for 1 hour 50 minutes. Then added dropwise allyl iodide (1.15 mL, 12.6 mmol) over 10 minutes and allowed to warm slowly from −78° C. to −55° C. over 5.25 hours. The RM was quenched by addition of aq sat NH₄Cl (15 mL) at −78° C. and stirred for 5 minutes. Then diluted with TBME (100 mL) and water (20 mL). The organic layer was separated. The aqueous layer was extracted with TBME (50 mL). The combined organic layers were dried over Na₂SO₄, filtered and concentrated under reduced pressure to give a yellow gum. The crude product was combined with another batch and purified by column chromatography (RediSep Column: Silica 120 g, eluent n-heptane:EtOAc 95:5 to 55:25 with a plateau at 25% for 9 min). The product containing fractions were combined and concentrated under reduced pressure to give a colourless foam (6.32 g, 99% pure, yield: 77%).

LC-MS: Rt=1.64 min; MS m/z [M+H−Boc]⁺ 511.2/513.2, MS m/z [M+H]⁺ 611.3/613.3; UPLC-MS1

### Step 4: tert-butyl (2R)-4-(2-bromo-5-(1-(oxiran-2-yl)propan-2-yl)-7-oxo-4-((2-(trimethylsilyl)ethoxy)methyl)-4,7-dihydro-[1,2,4]triazolo[1,5-a]pyrimidin-6-yl)-2-methylpiperazine-1-carboxylate

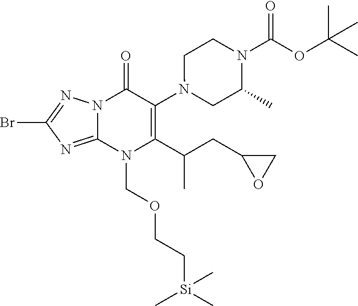 [SMILES: CC(CC1CO1)c1c(N2CCN(C(=O)OC(C)(C)C)[C@H](C)C2)c(=O)n2nc(Br)nc2n1COCC[Si](C)(C)C]

To tert-butyl (2R)-4-(2-bromo-7-oxo-5-(pent-4-en-2-yl)-4-((2-(trimethylsilyl)ethoxy)methyl)-4,7-dihydro-[1,2,4]triazolo[1,5-a]pyrimidin-6-yl)-2-methylpiperazine-1-carboxylate (6.32 g, 10.3 mmol) in DCM (100 mL) was added portionwise ethaneperoxoic acid (1.76 mL, 10.3 mmol) and the resulting mixture was stirred at RT in a stoppered flask for 20 hours. The RM was continued stirring at RT for 20 hours. The RM was diluted with DCM (50 mL) and poured into an ice-cold stirring solution of aq sat NaHCO₃ (200 mL) and 1M aq Na₂SO₃ (100 mL). The cooling bath was removed and the biphasic mixture was stirred vigorously for 5 minutes. The organic layer was separated and the aqueous layer was extracted with DCM (50 mL). The combined organic layers were washed with 1M aq Na₂SO₃ (20 mL) and brine (20 mL), dried over Na₂SO₄, filtered and concentrated under reduced pressure to give the title compound as a colourless foam (6.16 g, 92% pure, yield: 87%).

LC-MS: Rt=1.48/1.49 min; MS m/z [M+H−Boc]⁺ 527.2/529.2, MS m/z [M+H]⁺ 627.2/629.2; UPLC-MS 1

### Step 5: tert-butyl (2R)-4-(2-bromo-9-(hydroxymethyl)-7-methyl-5-oxo-5,7,8,9-tetrahydropyrrolo[1,2-c][1,2,4]triazolo[1,5-a]pyrimidin-6-yl)-2-methylpiperazine-1-carboxylate

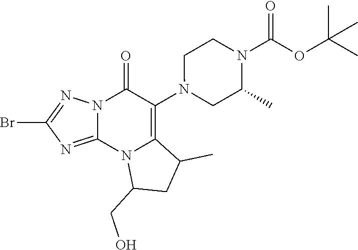 [SMILES: CC1CC(CO)n2c1c(N1CCN(C(=O)OC(C)(C)C)[C@H](C)C1)c(=O)n1nc(Br)nc21]

To tert-butyl (2R)-4-(2-bromo-5-(1-(oxiran-2-yl)propan-2-yl)-7-oxo-4-((2-(trimethylsilyl)ethoxy)methyl)-4,7-dihydro-[1,2,4]triazolo[1,5-a]pyrimidin-6-yl)-2-methylpiperazine-1-carboxylate (6.16 g, 9.81 mmol) in DMF (60 mL) was added water (1 mL) followed by KF (2.85 g, 49.1 mmol). The mixture was stirred at 48° C. for 1 hour. Continued stirring at 40° C. for 2 hours then at 35° C. for 72 hours. Continued stirring at 60° C. for 5 hours. The RM was poured onto ice (200 mL) and TBME (150 mL) was added. The mixture was stirred vigorously for 20 minutes. The organic layer was separated and the aqueous layer was extracted with TBME (2×100 mL). The organics were combined, dried over Na₂SO₄, filtered and concentrated under reduced pressure to give the title compound as a sticky yellow foam (mixture of diastereoisomers) (5.00 g, 90% pure, yield: 92%).

LC-MS: Rt=4.53/4.60/4.65 min; MS m/z [M+H−Boc]⁺ 397.1/399.1, m/z [M−H]⁻ 495.2/497.2; UPLC-MS 2

### Step 6: 2-bromo-6-((R)-4-(tert-butoxycarbonyl)-3-methylpiperazin-1-yl)-7-methyl-5-oxo-5,7,8,9-tetrahydropyrrolo[1,2-c][1,2,4]triazolo[1,5-a]pyrimidine-9-carboxylic acid

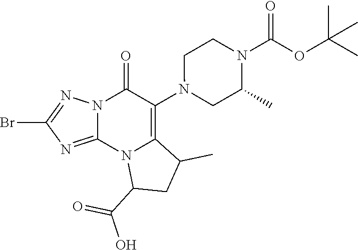 [SMILES: CC1CC(C(=O)O)n2c1c(N1CCN(C(=O)OC(C)(C)C)[C@H](C)C1)c(=O)n1nc(Br)nc21]

To tert-butyl (2R)-4-(2-bromo-9-(hydroxymethyl)-7-methyl-5-oxo-5,7,8,9-tetrahydropyrrolo[1,2-c][1,2,4]triazolo[1,5-a]pyrimidin-6-yl)-2-methylpiperazine-1-carboxylate (4.84 g, 8.76 mmol) in DCM (160 mL) and aq NaHCO₃ (159 mL, 88.0 mmol) at 0° C. were added KBr (104 mg, 876 μmol), N-methyl-N,N-dioctyloctan-1-aminium chloride (177 mg, 438 μmol) and TEMPO (14.0 mg, 88.0 μmol). The pH was adjusted to pH 8 by addition of 1M HCl, then added dropwise over 1 hour sodium hypochlorite (21.6 mL, 21.9 mmol) maintaining the pH below 8.5. The mixture was stirred at 0-2° C. for 2 hours. Sodium hypochlorite (21.6 mL, 21.9 mmol) was added dropwise over 30 minutes and the RM was stirred at 0-3° C. for 1 hour. Sodium hypochlorite (10.0 mL, 10.1 mmol) was added and the RM was continued stirring for 45 minutes. The RM was diluted with TBME (250 mL) and the biphasic mixture was stirred vigorously for 5 minutes. The aqueous layer was separated. The organic layer was extracted with aq sat NaHCO₃ (2×30 mL). The aqueous layers were combined and washed with TBME (100 mL). The aqueous layer was then diluted with DCM (200 mL). The biphasic mixture was stirred vigorously and slowly acidified to pH 2.8 by addition of 4N aq HCl with monitoring by a pH meter. The organic layer was separated and the aqueous layer was extracted with DCM (2×50 mL). The DCM layers were combined, dried over Na₂SO₄, filtered and concentrated under reduced pressure to give a cream-coloured foam (1.56 g). The crude product was purified by column chromatography (RediSep Column: Silica 40 g, eluent DCM:MeOH:AcOH 100:0:0 to 96:4:0.4). The product containing fractions were combined and azeotroped with toluene to give the title compound as an off-white foam (mixture of diastereoisomers) (850 mg, 99% pure, yield: 19%).

LC-MS: Rt=3.79/3.90/3.97 min; MS m/z [M+H−Boc]⁺411.1/413.1, MS m/z [M−H]⁻ 509.1/511.1; UPLC-MS 2

### Intermediate M1: tert-butyl (R)-4-((7S,9R)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)-2-methylpiperazine-1-carboxylate and Intermediate M2: tert-butyl (R)-4-((7R,9R)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)-2-methylpiperazine-1-carboxylate and Intermediate M3: tert-butyl (R)-4-((7R,9S)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)-2-methylpiperazine-1-carboxylate and Intermediate M4: tert-butyl (R)-4-((7S,9S)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)-2-methylpiperazine-1-carboxylate

### Step 1: tert-butyl (2R)-4-(2-bromo-9-((2-chloro-4-(trifluoromethyl)phenyl)carbamoyl)-7-methyl-5-oxo-5,7,8,9-tetrahydropyrrolo[1,2-c][1,2,4]triazolo[1,5-a]pyrimidin-6-yl)-2-methylpiperazine-1-carboxylate

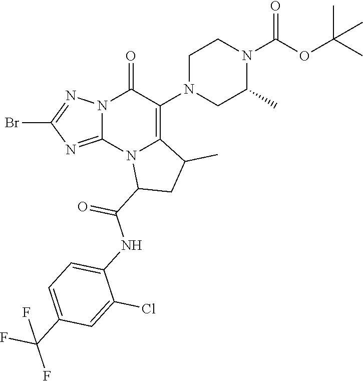 [SMILES: CC1CC(C(=O)Nc2ccc(C(F)(F)F)cc2Cl)n2c1c(N1CCN(C(=O)OC(C)(C)C)[C@H](C)C1)c(=O)n1nc(Br)nc21]

To 2-bromo-6-((R)-4-(tert-butoxycarbonyl)-3-methylpiperazin-1-yl)-7-methyl-5-oxo-5,7,8,9-tetrahydropyrrolo[1,2-c][1,2,4]triazolo[1,5-a]pyrimidine-9-carboxylic acid (Intermediate L) (850 mg, 1.66 mmol) in DCM (10 mL) at 0° C. were added 2-chloro-4-(trifluoromethyl)aniline (358 mg, 1.83 mmol) and Et₃N (1.39 mL, 9.97 mmol), followed by dropwise addition of T₃P 50% in EtOAc (1.98 mL, 3.32 mmol). The RM was stirred at RT overnight. 2-Chloro-4-(trifluoromethyl)aniline (100 mg, 511 μmol) and T₃P 50% in EtOAc (400 μL, 671 μmol) were added and the RM was continued stirring at RT for 4 hours. 2-Chloro-4-(trifluoromethyl)aniline (100 mg, 511 μmol), T₃P 50% in EtOAc (400 μL, 671 μmol) and Et₃N (500 μL, 3.59 mmol) were added and the RM stood in a stoppered flask in the fridge for 3 days. The RM was diluted with DCM (30 mL) and aq sat NaHCO₃ (50 mL) and the biphasic mixture was stirred vigorously at RT for 5 hours. The organic layer was separated and the aqueous layer was extracted with DCM (10 mL). The combined organic layers were washed with water (10 mL), filtered through a phase separator and concentrated under reduced pressure to give a brown gum (1.10 g). The crude product was purified by column chromatography (RediSep Column: Silica 2×24 g, eluent n-heptane:EtOAc 90:10 to 0:100). The product containing fractions were combined and concentrated under reduced pressure to give the title compound as a mixture of 4 diastereoisomers as a pale brown solid (550 mg, 95% pure, yield: 46%).

LC-MS: Rt=1.37/1.38 min; MS m/z [M+H−Boc]⁺ 588.1/590.1/592.1, m/z [M−H]⁻ 686.2/688.2/690.2; UPLC-MS 1

LC-MS: Rt=6.76/6.84/6.92/6.98 min; MS m/z [M+H−Boc]⁺588.1/590.1/592.0, m/z [M−H]⁻ 686.2/688.2/690.2; UPLC-MS 2

### Step 2: tert-butyl (R)-4-((7R,9S)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)-2-methylpiperazine-1-carboxylate and tert-butyl (R)-4-((7S,9R)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)-2-methylpiperazine-1-carboxylate and tert-butyl (R)-4-((7S,9S)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)-2-methylpiperazine-1-carboxylate and tert-butyl (R)-4-((7R,9R)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)-2-methylpiperazine-1-carboxylate

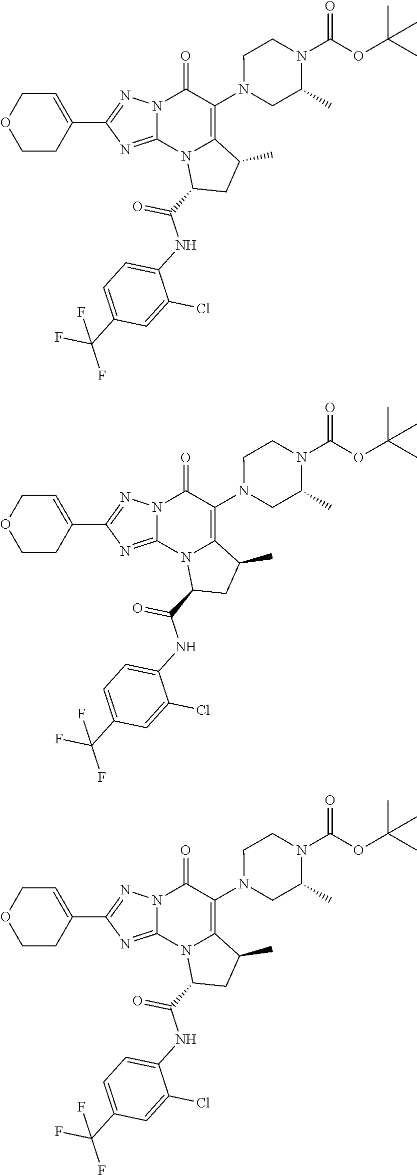 [SMILES: C[C@@H]1C[C@H](C(=O)Nc2ccc(C(F)(F)F)cc2Cl)n2c1c(N1CCN(C(=O)OC(C)(C)C)[C@H](C)C1)c(=O)n1nc(C3=CCOCC3)nc21 ; C[C@@H]1CN(c2c3n(c4nc(C5=CCOCC5)nn4c2=O)[C@H](C(=O)Nc2ccc(C(F)(F)F)cc2Cl)C[C@@H]3C)CCN1C(=O)OC(C)(C)C ; C[C@@H]1CN(c2c3n(c4nc(C5=CCOCC5)nn4c2=O)[C@@H](C(=O)Nc2ccc(C(F)(F)F)cc2Cl)C[C@@H]3C)CCN1C(=O)OC(C)(C)C]

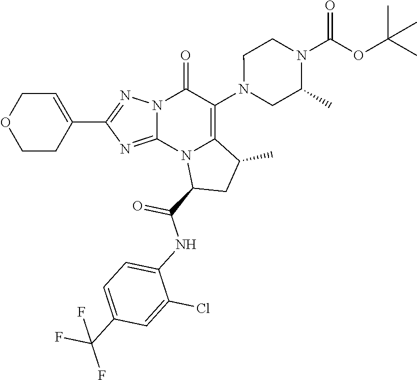 [SMILES: C[C@@H]1C[C@@H](C(=O)Nc2ccc(C(F)(F)F)cc2Cl)n2c1c(N1CCN(C(=O)OC(C)(C)C)[C@H](C)C1)c(=O)n1nc(C3=CCOCC3)nc21]

To tert-butyl (2R)-4-(2-bromo-9-((2-chloro-4-(trifluoromethyl)phenyl)carbamoyl)-7-methyl-5-oxo-5,7,8,9-tetrahydropyrrolo[1,2-c][1,2,4]triazolo[1,5-a]pyrimidin-6-yl)-2-methylpiperazine-1-carboxylate (550 mg, 798 μmol), PdCl₂(dppf)·DCM adduct (32.6 mg, 40.0 μmol) and K₃PO₄ (508 mg, 2.40 mmol) were added 1,4-dioxane (15 mL) and water (7 mL) and the RM was degassed and flushed with nitrogen. 2-(3,6-dihydro-2H-pyran-4-yl)-4,4,5,5-tetramethyl-1,3,2-dioxaborolane (201 mg, 958 μmol) was added. The RM was flushed again with nitrogen and stirred at 80° C. for 1 hour. The RM was allowed to cool to RT, then diluted with EtOAc (50 mL) and water (10 mL). The organic layer was separated. The aqueous layer was extracted with EtOAc (2×20 mL). The combined organic layers were mixed with Si-Thiol (2 g) and stirred at 40° C. for 10 minutes. The mixture was filtered, dried over Na₂SO₄, filtered and concentrated under reduced pressure to give a yellow foam (750 mg). The crude product was purified by column chromatography (RediSep Column: Silica 24 g+12 g, eluent n-heptane:EtOAc 90:10 to 0:100). The product containing fractions were combined and concentrated under reduced pressure to give the title compound as a mixture of 4 diastereoisomers as a pale yellow solid (403 mg, yield: 73%). The mixture was purified by preparative chiral SFC (instrument: Sepiatec prep SFC100; column: OVEN4 Chiralpak IC 250 mm×30 mm 5 μm; eluent: A: 40% IPA+0.1% NH₃, B: 60% scCO₂; flow rate: 80.0 mL/min; detection: UV; injection volume: 0.8 mL; gradient: isocratic A: 40%, B: 60%; oven temperature: 40° C.; BPR: 110 bar).

Peak 1: tert-butyl (R)-4-((7S,9R)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)-2-methylpiperazine-1-carboxylate (Intermediate M1)

off-white solid, 51.8 mg, 99% pure, yield: 9%

LC-MS: Rt=6.56 min; MS m/z [M+H−Boc]⁺592.2/594.2, m/z [M−H]⁻ 690.3/692.3; UPLC-MS 2

Chiral HPLC (C-HPLC 7): Rt=1.53 min, 99.5% ee

Peak 2: tert-butyl (R)-4-((7R,9R)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)-2-methylpiperazine-1-carboxylate (Intermediate M2)

off-white solid, 118 mg, 99% pure, yield: 21%

LC-MS: Rt=6.74 min; MS m/z [M+H−Boc]⁺ 592.2/594.2, m/z [M−H]⁻ 690.3/692.3; UPLC-MS 2

Chiral HPLC (C-HPLC 7): Rt=2.08 min, 99.5% ee

Peak 3: tert-butyl (R)-4-((7R,9S)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)-2-methylpiperazine-1-carboxylate (Intermediate M3)

off-white solid, 102 mg, 99% pure, yield: 18%

LC-MS: Rt=6.64 min; MS m/z [M+H−Boc]⁺ 592.2/594.2, m/z [M−H]⁻ 690.3/692.3; UPLC-MS 2

Chiral HPLC (C-HPLC 7): Rt=2.84 min, 99.5% ee

Peak 4: tert-butyl (R)-4-((7S,9S)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)-2-methylpiperazine-1-carboxylate (Intermediate M4)

off-white solid, 60.0 mg, 99% pure, yield: 11%

LC-MS: Rt=6.80 min; MS m/z [M+H−Boc]⁺592.2/594.2, m/z [M−H]⁻ 690.3/692.3; UPLC-MS 2

Chiral HPLC (C-HPLC 7): Rt=4.11 min, 99.5% ee

### Intermediate N1: (7S,9S)—N-(2-chloro-4-(trifluoromethyl)phenyl)-2-(3,6-dihydro-2H-pyran-4-yl)-7-methyl-6-((R)-3-methylpiperazin-1-yl)-5-oxo-5,7,8,9-tetrahydropyrrolo[1,2-c][1,2,4]triazolo[1,5-a]pyrimidine-9-carboxamide

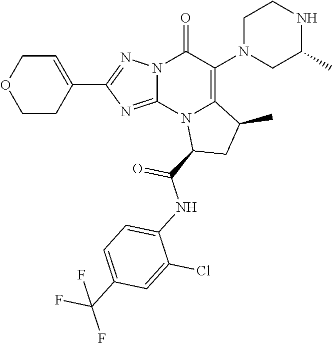 [SMILES: C[C@@H]1CN(c2c3n(c4nc(C5=CCOCC5)nn4c2=O)[C@H](C(=O)Nc2ccc(C(F)(F)F)cc2Cl)C[C@@H]3C)CCN1]

Tert-butyl (R)-4-((7S,9S)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)-2-methylpiperazine-1-carboxylate (Intermediate M41 (60.0 mg, 87.0 μmol) was dissolved in 4N HCl in 1,4-dioxane (2.00 mL, 65.8 mmol) and stood at RT in a stoppered flask for 2 hours. The RM was concentrated under reduced pressure to give the title compound as a colourless solid (directly used in the next step—see Example 18B).

LC-MS: Rt=0.78 min; MS m/z [M+H]⁺ 592.3/594.3, m/z [M−H]⁻ 590.2/592.2; UPLC-MS 2

### Intermediate N2: (7R,9R)—N-(2-chloro-4-(trifluoromethyl)phenyl)-2-(3,6-dihydro-2H-pyran-4-yl)-7-methyl-6-((R)-3-methylpiperazin-1-yl)-5-oxo-5,7,8,9-tetrahydropyrrolo[1,2-c][1,2,4]triazolo[1,5-a]pyrimidine-9-carboxamide

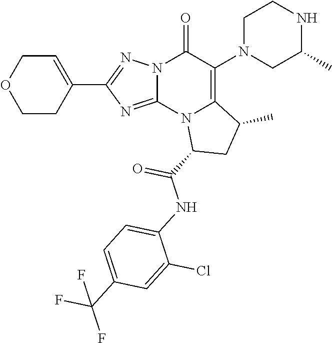 [SMILES: C[C@@H]1CN(c2c3n(c4nc(C5=CCOCC5)nn4c2=O)[C@@H](C(=O)Nc2ccc(C(F)(F)F)cc2Cl)C[C@H]3C)CCN1]

Tert-butyl (R)-4-((7R,9R)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)-2-methylpiperazine-1-carboxylate (Intermediate M2) (118 mg, 170 μmol) was dissolved in 4N HCl in 1,4-dioxane (2.00 mL, 65.8 mmol) and stood at RT in a stoppered flask for 2 hours. The RM was concentrated under reduced pressure to give the title compound as a colourless solid (directly used in the next step—see Example 18A).

LC-MS: Rt=0.82 min; MS m/z [M+H]⁺ 592.4/594.3, m/z [M−H]⁻ 590.3/592.3; UPLC-MS 2

### Intermediate O1: tert-butyl (S)-4-(2-(3,6-dihydro-2H-pyran-4-yl)-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate and Intermediate O2: tert-butyl (R)-4-(2-(3,6-dihydro-2H-pyran-4-yl)-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate

### Step 1: tert-butyl 4-(2-bromo-5-(4-ethoxy-4-oxobutyl)-7-oxo-4,7-dihydro-[1,2,4]triazolo[1,5-a]pyrimidin-6-yl)piperazine-1-carboxylate

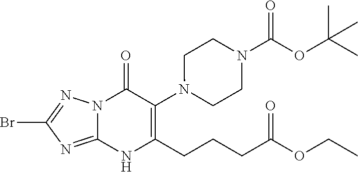 [SMILES: CCOC(=O)CCCc1[nH]c2nc(Br)nn2c(=O)c1N1CCN(C(=O)OC(C)(C)C)CC1]

To a stirred solution of 3-bromo-1H-1,2,4-triazol-5-amine (Intermediate AI) (2.23 g, 13.7 mmol) and diethyl 2-(4-(tert-butoxycarbonyl)piperazin-1-yl)-3-oxoheptanedioate (Intermediate AF) (5.67 g, 13.7 mmol) in toluene (50 mL) was added pTsOH (3.90 g, 20.5 mmol) at RT and the RM was stirred at 110° C. for 72 hours. The RM was cooled to RT, Et₃N (9.54 mL, 68.4 mmol) was added, followed by di-tert-butyl dicarbonate (3.18 mL, 13.7 mmol) in DCM (50 mL) and the RM was stirred at RT for 3 hours. The RM was concentrated, then redissolved in DCM. The solution was washed twice with water. The combined organic layers were dried through a phase separator and concentrated under reduced pressure. The residue was dissolved in DCM and washed with 5% aq NaHCO₃. The organic layer was dried through a phase separator and concentrated under reduced pressure to afford a brown solid. The crude product was purified by column chromatography (RediSep Column: Silica 80 g, eluent DCM:MeOH 100:0 to 90:10). The product containing fractions were combined and concentrated under reduced pressure to give the title compound as a brown solid (2.60 g, 90% pure, yield: 33%).

LC-MS: Rt=1.01 min; MS m/z [M+H]⁺ 513.2/515.2, m/z [M−H]⁻ 511.4/513.4; UPLC-MS 6

### Step 2: rac-ethyl 2-bromo-6-(4-(tert-butoxycarbonyl)piperazin-1-yl)-5-oxo-5,7,8,9-tetrahydropyrrolo[1,2-c][1,2,4]triazolo[1,5-a]pyrimidine-9-carboxylate

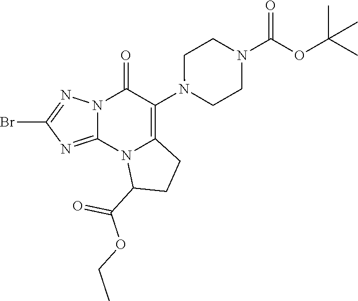 [SMILES: CCOC(=O)C1CCc2c(N3CCN(C(=O)OC(C)(C)C)CC3)c(=O)n3nc(Br)nc3n21]

A colourless solution of tert-butyl 4-(2-bromo-5-(4-ethoxy-4-oxobutyl)-7-oxo-4,7-dihydro-[1,2,4]triazolo[1,5-a]pyrimidin-6-yl)piperazine-1-carboxylate (1.00 g, 1.75 mmol) in THF (10 mL) was cooled to −78° C. Then a solution of LiHMDS 1M in THF (12.3 mL, 12.3 mmol) was added dropwise and the RM was stirred at −78° C. for 2 hours. Then chlorotrimethylsilane (1.12 mL, 8.77 mmol) was added and the RM was stirred at −78° C. for 2 hours. NBS (374 mg, 2.10 mmol) was added at −78° C. and the RM was stirred at −78° C. for 30 minutes. The RM was quenched with water and extracted twice with EtOAc. The organic layer was dried through a phase separator and concentrated under reduced pressure. The crude product was purified by column chromatography (RediSep Column: Silica 40 g, eluent cyclohexane:EtOAc 100:0 to 0:100). The product containing fractions were combined and concentrated under reduced pressure to give the title compound as a brown solid (418 mg, 99% pure, yield: 47%).

LC-MS: Rt=0.97 min; MS m/z [M+H−Boc]⁺ 411.2/413.2, m/z [M+H]⁺ 511.3/513.3; UPLC-MS 6

### Step 3: rac-2-bromo-6-(4-(tert-butoxycarbonyl)piperazin-1-yl)-5-oxo-5,7,8,9-tetrahydropyrrolo[1,2-c][1,2,4]triazolo[1,5-a]pyrimidine-9-carboxylic acid

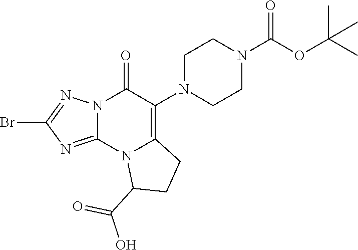 [SMILES: CC(C)(C)OC(=O)N1CCN(c2c3n(c4nc(Br)nn4c2=O)C(C(=O)O)CC3)CC1]

To a solution of rac-ethyl 2-bromo-6-(4-(tert-butoxycarbonyl)piperazin-1-yl)-5-oxo-5,7,8,9-tetrahydropyrrolo[1,2-c][1,2,4]triazolo[1,5-a]pyrimidine-9-carboxylate (418 mg, 817 μmol) in THF (3 mL) and water (1.5 mL) was added LiOH·H2O (37.7 mg, 899 μmol). The RM was stirred at RT for 5 hours. The solution was diluted with water and extracted with EtOAc. The aqueous layer was acidified with a 1N aq HCl and extracted twice with EtOAc. The combined organic layers were dried through a phase separator and concentrated under reduced pressure to give the title compound as a white solid (330 mg, 99% pure, yield: 84%).

LC-MS: Rt=0.64 min; MS m/z [M+H−Boc]⁺383.1/385.1, m/z [M−H]⁻ 481.4/483.2; UPLC-MS 6

### Step 4: rac-tert-butyl 4-(2-bromo-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate

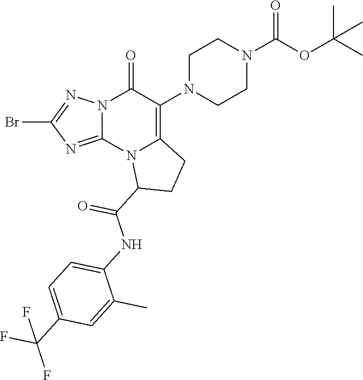 [SMILES: Cc1cc(C(F)(F)F)ccc1NC(=O)C1CCc2c(N3CCN(C(=O)OC(C)(C)C)CC3)c(=O)n3nc(Br)nc3n21]

To a solution of rac-2-bromo-6-(4-(tert-butoxycarbonyl)piperazin-1-yl)-5-oxo-5,7,8,9-tetrahydropyrrolo[1,2-c][1,2,4]triazolo[1,5-a]pyrimidine-9-carboxylic acid (300 mg, 621 μmol) in DCM (10 mL) was added HATU (354 mg, 931 μmol). The RM was stirred for 15 minutes. 2-Methyl-4-(trifluoromethyl)aniline (132 μL, 931 μmol) was added followed by BEMP (449 μL, 1.55 mmol). The resulting RM was stirred at 30° C. for 16 hours. The RM was diluted with DCM and washed with water. The aqueous layer was extracted with EtOAc. The combined organic layers were dried through a phase separator and concentrated under reduced pressure. The crude product was purified by column chromatography (RediSep Column: Silica 12 g, eluent DCM:MeOH 100:0 to 95:5) to give the title compound (145 mg, 70% pure, yield: 25.5%). The impure fractions were further purified by reverse phase preparative HPLC (RP-HPLC acidic 1: 30 to 100% B in 20 min). The product containing fractions were combined and ACN was removed. The residue was basified with aq NaHCO₃ and extracted with EtOAc. The organic layer was dried through a phase separator and concentrated under reduced pressure to give the title compound as a white solid (88.0 mg, 96% pure yield: 22%).

Total: 233 mg, 80% pure, yield: 48%

LC-MS: Rt=1.29 min; MS m/z [M+H−Boc]⁺ 540.1/542.1, m/z [M−H]⁻ 638.4/640.4; UPLC-MS 6

### Step 5: tert-butyl (S)-4-(2-(3,6-dihydro-2H-pyran-4-yl)-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate and tert-butyl (R)-4-(2-(3,6-dihydro-2H-pyran-4-yl)-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate

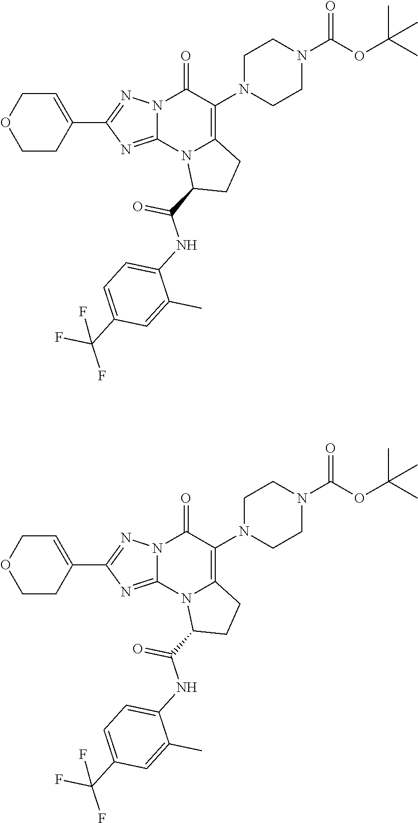 [SMILES: Cc1cc(C(F)(F)F)ccc1NC(=O)[C@@H]1CCc2c(N3CCN(C(=O)OC(C)(C)C)CC3)c(=O)n3nc(C4=CCOCC4)nc3n21 ; Cc1cc(C(F)(F)F)ccc1NC(=O)[C@H]1CCc2c(N3CCN(C(=O)OC(C)(C)C)CC3)c(=O)n3nc(C4=CCOCC4)nc3n21]

Rac-tert-butyl 4-(2-bromo-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate (233 mg, 327 μmol), 2-(3,6-dihydro-2H-pyran-4-yl)-4,4,5,5-tetramethyl-1,3,2-dioxaborolane (103 mg, 491 μmol), aq K₃PO₄ (437 μL, 655 μmol) and XPhos Pd G3 (27.7 mg, 33.0 μmol) in 1,4-dioxane (3 mL) was degassed with argon and then stirred at 100° C. for 1 hour. The RM was diluted with EtOAc and washed with water. The organic layer was dried through a phase separator and concentrated under reduced pressure. The crude product was purified by column chromatography (RediSep Column: Silica 24 g gold, eluent DCM:MeOH 100:0 to 95:5). The product containing fractions were combined and concentrated under reduced pressure to give the title compound as a yellow solid (96.0 mg, 85% pure, yield: 39%). The racemate (96.0 mg) was purified by preparative chiral HPLC (instrument: Waters Prep SFC100 MS; column: Chiralpak IB-N 250 mm×30 mm 5 μm; eluent: A: 35% MeOH+0.1% NH₃, B: 65% scCO₂; flow rate: 80.0 mL/min; detection: DAD; injection volume: 3 mL; gradient: isocratic A: 35%, B: 65%; oven temperature: 40° C.; BPR: 120 bar).

Peak 1: tert-butyl (S)-4-(2-(3,6-dihydro-2H-pyran-4-yl)-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate (Intermediate O1)

32.0 mg, 99% pure, yield: 39%

LC-MS: Rt=1.23 min; MS m/z [M+H−Boc]⁺ 544.4, MS m/z [M−H]⁻ 642.4; UPLC-MS 1

Chiral HPLC (C-HPLC 11): Rt=0.83 min, 99.5% ee

Peak 2: tert-butyl (R)-4-(2-(3,6-dihydro-2H-pyran-4-yl)-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate (Intermediate O2)

27.0 mg, 99% pure, yield: 33%

LC-MS: Rt=1.23 min; MS m/z [M+H−Boc]⁺ 544.4, MS m/z [M−H]⁻ 642.4; UPLC-MS 1

Chiral HPLC (C-HPLC 11): Rt=3.78 min, 99.5% ee

### Intermediate P1: (S)-2-(3,6-dihydro-2H-pyran-4-yl)-N-(2-methyl-4-(trifluoromethyl)phenyl)-5-oxo-6-(piperazin-1-yl)-5,7,8,9-tetrahydropyrrolo[1,2-c][1,2,4]triazolo[1,5-a]pyrimidine-9-carboxamide

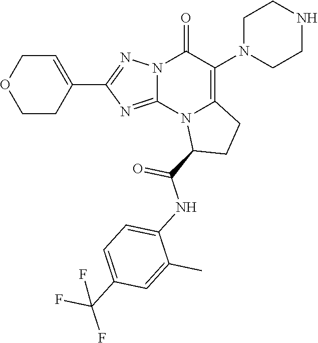 [SMILES: Cc1cc(C(F)(F)F)ccc1NC(=O)[C@@H]1CCc2c(N3CCNCC3)c(=O)n3nc(C4=CCOCC4)nc3n21]

To a solution of tert-butyl (S)-4-(2-(3,6-dihydro-2H-pyran-4-yl)-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate (Intermediate O1) (36.5 mg, 57.0 μmol) in MeOH (1 mL) was added 4N HCl in 1,4-dioxane (142 μL, 567 μmol). The RM was stirred at RT for 16 hours. 4N HCl in 1,4-dioxane (710 μL, 2.84 mmol) was added and the RM was stirred at RT for 24 hours. The RM was concentrated under reduced pressure to give the title compound (31.0 mg, 99% pure, yield: 99%).

LC-MS: Rt=1.35 min; MS m/z [M+H]⁺ 544.4, MS m/z [M−H]⁻ 542.2; UPLC-MS 7

### Intermediate P2: (R)-2-(3,6-dihydro-2H-pyran-4-yl)-N-(2-methyl-4-(trifluoromethyl)phenyl)-5-oxo-6-(piperazin-1-yl)-5,7,8,9-tetrahydropyrrolo[1,2-c][1,2,4]triazolo[1,5-a]pyrimidine-9-carboxamide

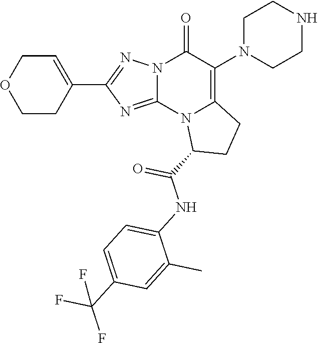 [SMILES: Cc1cc(C(F)(F)F)ccc1NC(=O)[C@H]1CCc2c(N3CCNCC3)c(=O)n3nc(C4=CCOCC4)nc3n21]

To a solution of tert-butyl (R)-4-(2-(3,6-dihydro-2H-pyran-4-yl)-9-((2-methyl-4-(trifluoromethyl)phenyl)carbamoyl)-5-oxo-5,7,8,9-tetrahydropyrrolo[1,2-c][1,2,4]triazolo[1,5-a]pyrimidin-6-yl)piperazine-1-carboxylate (Intermediate O2) (36.5 mg, 57.0 μmol) in MeOH (1 mL) was added 4N HCl in 1,4-dioxane (1.00 mL, 4.00 mmol). The RM was stirred at RT for 18 hours. The RM was concentrated under reduced pressure to give the title compound (32.0 mg, 97% pure, yield: 99%).

LC-MS: Rt=1.40 min; MS m/z [M+H]⁺ 544.4, MS m/z [M−H]⁻ 542.2; UPLC-MS 7

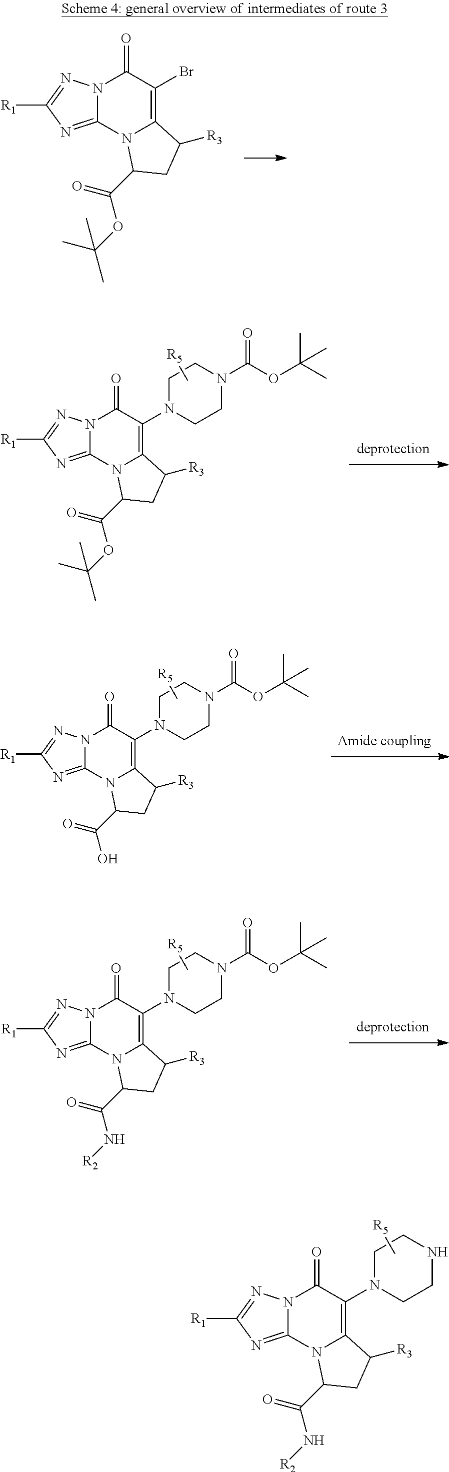

### Intermediate Q: (7R)-6-((1S,6S)-2,5-diazabicyclo[4.2.0]octan-2-yl)-N-(2-chloro-4-(trifluoromethyl) phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide

### Step 1: tert-butyl (R)-4-(1-amino-2-imino-6-oxo-1,2,3,6-tetrahydropyrimidin-4-yl)pentanoate

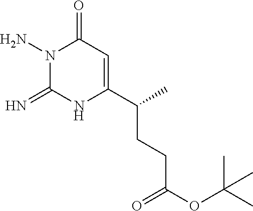

Hydrazinecarboximidamide·HCl (21.0 g, 190 mmol) was suspended in MeOH (400 mL) and sodium methanolate 5.4 molar (38.0 mL, 200 mmol) was added. After 15 minutes at RT was added 7-(tert-butyl) 1-methyl (R)-4-methyl-3-oxoheptanedioate (Intermediate AG) (25.0 g, 97.0 mmol) and the RM was stirred at RT for 14 hours. Hydrazinecarboximidamide·HCl (21.0 g, 190 mmol) was added and the RM was stirred at RT for 2 hours. The RM was quenched with water, diluted with EtOAc and water, extracted twice with EtOAc and the combined organic layers were dried over Na₂SO₄ and concentrated under reduced pressure. The crude product was purified by column chromatography (RediSep Column: Silica 120 g, eluent DCM:DCM/MeOH (8/2) 100:0 to 40:60). The product containing fractions were combined and concentrated under reduced pressure to give the title compound as a white solid (15.5 g, 100% pure, yield: 57%).

LC-MS: Rt=0.68 min; MS m/z [M+H]⁺ 283.3; UPLC-MS 1

### Step 2: tert-butyl (R)-4-(2-(3,6-dihydro-2H-pyran-4-yl)-7-oxo-4,7-dihydro-[1,2,4]triazolo[1,5-a]pyrimidin-5-yl)pentanoate

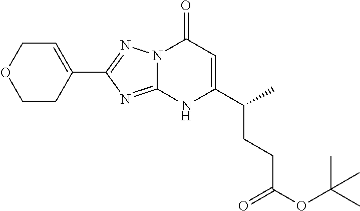

Tert-butyl (R)-4-(1-amino-2-imino-6-oxo-1,2,3,6-tetrahydropyrimidin-4-yl)pentanoate (15.5 g, 55.0 mmol), NMP (40 mL) and iron(III)chloride (17.9 g, 110 mmol) were added to 3,6-dihydro-2H-pyran-4-carbaldehyde (Intermediate AB) (1 g/mL in NMP) (9.26 g, 82.6 mmol) and the RM was stirred at 50° C. for 20 hours under air. 3,6-Dihydro-2H-pyran-4-carbaldehyde (Intermediate AB) (1 g/mL in NMP) (6.17 g, 55.0 mmol) was added and the RM was stirred at 50° C. for 48 hours. The RM was quenched with water, then it was diluted with DCM and water, extracted twice with DCM and the combined organic layers were dried over Na₂SO₄ and concentrated under reduced pressure to afford a brown oil. The crude product was purified twice by column chromatography (RediSep Column: Silica 220 g, eluent DCM:DCM/MeOH (8/2) 100:0 to 45:55), (RediSep Column: Silica 80 g, eluent heptane:EtOAc 100:0 to 0:100). The product containing fractions were combined and concentrated under reduced pressure. The residue was triturated in Et₂O to give the title compound as a white solid (10.1 g, 100% pure, yield: 49%).

LC-MS: Rt=0.77 min; MS m/z [M+H]⁺ 375.5, MS m/z [M−H]⁻ 373.4; UPLC-MS 1

LC-MS: Rt=3.71 min; MS m/z [M+H]⁺ 375.3, MS m/z [M−H]⁻ 373.3; UPLC-MS 2

### Step 3: tert-butyl (7R,9R)-6-bromo-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylate and tert-butyl (7S,9S)-6-bromo-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylate and tert-butyl (7S,9R)-6-bromo-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylate and tert-butyl (7R,9S)-6-bromo-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylate

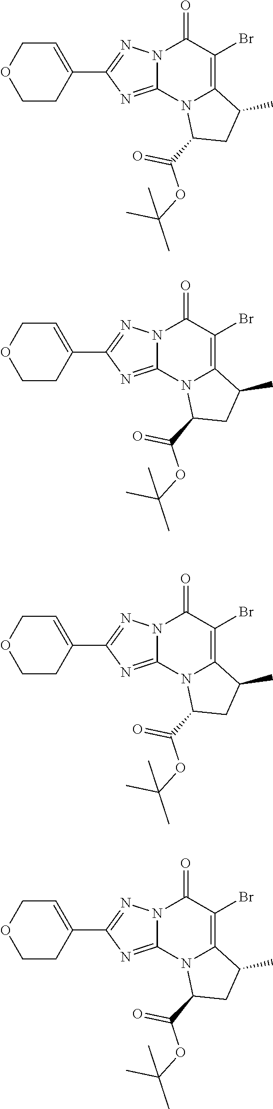

LiHMDS 1M (57.6 mL, 57.6 mmol) was added at −78° C. to a stirred solution of tert-butyl (R)-4-(2-(3,6-dihydro-2H-pyran-4-yl)-7-oxo-4,7-dihydro-[1,2,4]triazolo[1,5-a]pyrimidin-5-yl)pentanoate (7.19 g, 19.2 mmol) in THF (200 mL) and the RM was stirred at −78° C. for 2 hours. NBS (5.13 g, 28.8 mmol) in THF (60 mL) was then added dropwise. The RM was stirred for 15 minutes. NBS (5.13 g, 28.8 mmol) in THF (60 mL) was added and the RM was stirred for 1.75 hours. The RM was quenched with aq sat NH₄Cl. The RM was diluted with EtOAc and 10% aq Na₂S₂O₃, extracted twice with EtOAc and the combined organic layers were dried over Na₂SO₄ and concentrated under reduced pressure. The crude product was purified twice by column chromatography (RediSep Column: Silica 80 g, eluent heptane:EtOAc 100:0 to 10:90), (RediSep Column: Silica 120 g, eluent heptane:EtOAc 100:0 to 40:60). All fractions containing peak 1 were combined and concentrated under reduced pressure. The residue was triturated in Et₂O to give the title compound (mixture of tert-butyl (7S,9R)-6-bromo-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylate and tert-butyl (7R,9S)-6-bromo-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylate) as a white solid (600 mg, 70% pure, yield: 5%).

LC-MS: Rt=0.84 min; MS m/z [M+H]⁺ 451.3/453.3; UPLC-MS 1

LC-MS: Rt=3.98 min; MS m/z [M+H]⁺ 451.1/453.1; UPLC-MS 2

All fractions containing peak 2 were combined and concentrated under reduced pressure. The residue was triturated in Et₂O to give the title compound (6:4 mixture of tert-butyl (7R,9R)-6-bromo-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylate and tert-butyl (7S,9S)-6-bromo-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylate) as a white solid (4.40 g, 100% pure, yield: 51%).

LC-MS: Rt=0.88 min; MS m/z [M+H]⁺ 451.2/453.2; UPLC-MS 1

LC-MS: Rt=4.08 min; MS m/z [M+H]⁺ 451.1/453.1; UPLC-MS 2

Peak 2 (2.00 g) (6:4 mixture of tert-butyl (7R,9R)-6-bromo-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylate and tert-butyl (7S,9S)-6-bromo-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylate) was purified by preparative chiral HPLC (instrument: analytical SFC-MS Waters UPC²; column: OVEN2 Chiralpak AD-H 250 mm×30 mm 5 μm; eluent: A: 20% 0.05% NH₃ in MeOH, B: 80% scCO₂; flow rate: 85.0 mL/min; detection: UV; injection volume: 0.9 mL; gradient: isocratic A: 20%, B: 80%; oven temperature: 40° C.; BPR: 130 bar).

Peak 2.1: tert-butyl (7R,9R)-6-bromo-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylate

804 mg, 100% pure, yield: 9%

LC-MS: Rt=0.86 min; MS m/z [M+H]⁺ 451.2/453.2; UPLC-MS 1

LC-MS: Rt=4.04 min; MS m/z [M+H]⁺ 451.2/453.2; UPLC-MS 2

Chiral HPLC (C-HPLC 12): Rt=1.15 min, 99.5% ee

Peak 2.2: tert-butyl (7S,9S)-6-bromo-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylate

388 mg, 100% pure, yield: 4.%

LC-MS: Rt=0.85 min; MS m/z [M+H]⁺ 451.3/453.3; UPLC-MS 1

LC-MS: Rt=4.07 min; MS m/z [M+H]⁺ 451.1/453.1; UPLC-MS 2

Chiral HPLC (C-HPLC 12): Rt=1.60 min, 99.5% ee

### Step 4: tert-butyl (7R)-6-((1S,6S)-5-(tert-butoxycarbonyl)-2,5-diazabicyclo[4.2.0]octan-2-yl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylate

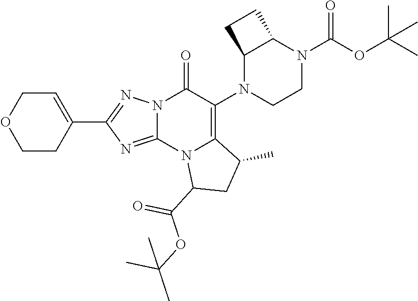 [SMILES: C[C@@H]1CC(C(=O)OC(C)(C)C)n2c1c(N1CCN(C(=O)OC(C)(C)C)[C@H]3CC[C@@H]31)c(=O)n1nc(C3=CCOCC3)nc21]

Potassium acetate (362 mg, 3.69 mmol) was added at RT to a stirred solution of tert-butyl (7R,9R)-6-bromo-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylate (Peak 2.1) (555 mg, 1.23 mmol) and tert-butyl (1S,6S)-2,5-diazabicyclo[4.2.0]octane-2-carboxylate (Intermediate AH) (783 mg, 3.69 mmol) in ACN (5 mL) and the RM was stirred at 90° C. for 4 days. The RM was diluted with EtOAc and water, extracted twice with EtOAc and the combined organic layers were washed with brine, dried over Na₂SO₄ and concentrated under reduced pressure. The crude product was purified by column chromatography (RediSep Column: Silica 40 g, eluent heptane:EtOAc 100:0 to 0:100). The product containing fractions were combined and concentrated under reduced pressure to give the title compound (mixture of cis/trans) as a white foam (493 mg, 73% pure, yield: 50%).

LC-MS: Rt=1.25 min; MS m/z [M+H]⁺ 583.5; UPLC-MS 1

LC-MS: Rt=5.93 min; MS m/z [M+H]⁺ 583.4; UPLC-MS 2

Alternatively, intermediate AH was added to a stirred solution of tert-butyl (7R,9R)-6-bromo-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylate (Peak 2.1) in DMSO and diisopropylamine, and stirred at 60° C. for about 50 hours. The reaction mixture was diluted in EtOAc/water, extracted twice with EtOAc and the combined organic extracts washed with brine, dried over Na₂SO₄ and concentrated.

### Step 5: (7R)-6-((1S,6S)-5-(tert-butoxycarbonyl)-2,5-diazabicyclo[4.2.0]octan-2-yl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylic acid

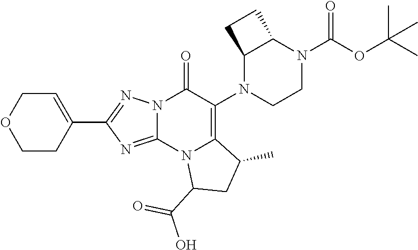 [SMILES: C[C@@H]1CC(C(=O)O)n2c1c(N1CCN(C(=O)OC(C)(C)C)[C@H]3CC[C@@H]31)c(=O)n1nc(C3=CCOCC3)nc21]

TFA (2.00 mL, 26.0 mmol) was added at RT to tert-butyl (7R)-6-((1S,6S)-5-(tert-butoxycarbonyl)-2,5-diazabicyclo[4.2.0]octan-2-yl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylate (493 mg, 618 μmol) and the RM was stirred at RT for 5 hours. The RM was concentrated, triturated in Et₂O and reconcentrated to afford a white solid. This solid was dissolved in DCM (10 mL) and Et₃N (129 μL, 926 μmol) was added, followed by Boc₂O (213 μL, 926 μmol). The RM was stirred at RT for 1 hour. The RM was diluted with DCM and water, pH was adjusted to ˜ 2 with 0.1M HCl and the aqueous layer was extracted twice with DCM. The combined organic layers were washed with brine, dried over Na₂SO₄ and concentrated under reduced pressure to afford an off-white solid. The crude product was purified by column chromatography (RediSep Column: Silica 24 g, eluent DCM:DCM/MeOH (8/2) 100:0 to 50:50). The product containing fractions were combined and concentrated under reduced pressure to give the title compound (mixture of cis/trans) as a white foam (388 mg, 85% pure, yield: 100%).

LC-MS: Rt=0.88/0.91 min; MS m/z [M+H]⁺ 527.4, MS m/z [M−H]⁻ 525.3; UPLC-MS 1

LC-MS: Rt=4.19/4.37 min; MS m/z [M+H]⁺ 527.3, MS m/z [M−H]⁻ 525.3; UPLC-MS 2

### Step 6: tert-butyl (1S,6S)-5-((7R)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)-2,5-diazabicyclo[4.2.0]octane-2-carboxylate

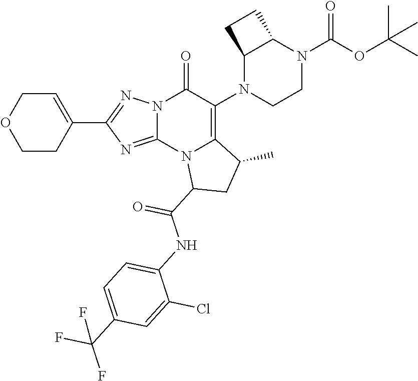 [SMILES: C[C@@H]1CC(C(=O)Nc2ccc(C(F)(F)F)cc2Cl)n2c1c(N1CCN(C(=O)OC(C)(C)C)[C@H]3CC[C@@H]31)c(=O)n1nc(C3=CCOCC3)nc21]

To a stirred solution of (7R)-6-((1S,6S)-5-(tert-butoxycarbonyl)-2,5-diazabicyclo[4.2.0]octan-2-yl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxylic acid (388 mg, 626 μmol) and 2-chloro-4-(trifluoromethyl)aniline (159 mg, 814 μmol) was added at RT pyridine (507 μL, 6.26 mmol) then T3P 50% in EtOAc (746 μL, 1.25 mmol) and the RM was stirred at RT for 1 day. The RM was diluted with DCM and water, extracted twice with DCM and the combined organic layers were washed with brine, dried over Na₂SO₄ and concentrated under reduced pressure. The crude product was purified by column chromatography (RediSep Column: Silica 24 g, eluent DCM:DCM/MeOH (8/2) 100:0 to 50:50). The product containing fractions were combined and concentrated under reduced pressure to give the title compound (mixture of cis/trans) as a white solid (420 mg, 86% pure, yield: 82%).

LC-MS: Rt=1.39 min; MS m/z [M+H]⁺ 704.4/706.3, MS m/z [M−H]⁻ 702.3/704.4; UPLC-MS 1

LC-MS: Rt=6.50/6.59 min; MS m/z [M+H]⁺ 704.3/706.3, MS m/z [M−H]⁻ 702.3/704.4; UPLC-MS 2

### Step 7: (7R)-6-((1S,6S)-2,5-diazabicyclo[4.2.0]octan-2-yl)-N-(2-chloro-4-(trifluoromethyl)phenyl)-2-(3,6-dihydro-2H-pyran-4-yl)-7-methyl-5-oxo-5,7,8,9-tetrahydropyrrolo[1,2-c][1,2,4]triazolo[1,5-a]pyrimidine-9-carboxamide

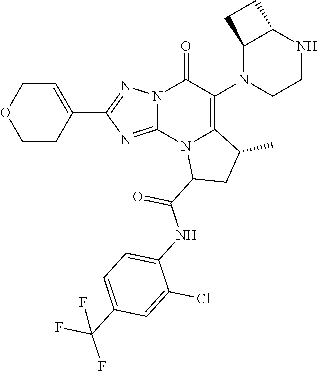 [SMILES: C[C@@H]1CC(C(=O)Nc2ccc(C(F)(F)F)cc2Cl)n2c1c(N1CCN[C@H]3CC[C@@H]31)c(=O)n1nc(C3=CCOCC3)nc21]

Hydrogen chloride 4N in 1,4-dioxane (3.00 mL, 12.0 mmol) was added at RT to tert-butyl (1S,6S)-5-((7R)-9-((2-chloro-4-(trifluoromethyl)phenyl)carbamoyl)-2-(3,6-dihydro-2H-pyran-4-yl)-7-methyl-5-oxo-5,7,8,9-tetrahydropyrrolo[1,2-c][1,2,4]triazolo[1,5-a]pyrimidin-6-yl)-2,5-diazabicyclo[4.2.0]octane-2-carboxylate (420 mg, 513 μmol) and the RM was stirred at RT for 30 minutes. The RM was diluted with 10% MeOH in DCM and aq NaHCO₃ and extracted twice with DCM. The combined organic layers were dried over Na₂SO₄ and concentrated under reduced pressure. The crude product was purified by column chromatography (RediSep Column: Silica 12 g, eluent DCM:DCM/MeOH/NH₃ (80/18/2) 100:0 to 85:15). The product containing fractions were combined and concentrated under reduced pressure to give the title compound (mixture of cis/trans) as a white solid (307 mg, 94% pure, yield: 93%).

LC-MS: Rt=0.83 min; MS m/z [M+H]⁺ 604.2/606.2, MS m/z [M−H]⁻ 602.3/604.2; UPLC-MS 1

LC-MS: Rt=3.96/4.03 min; MS m/z [M+H]⁺ 604.3/606.3, MS m/z [M−H]⁻ 602.4/604.4; UPLC-MS 2

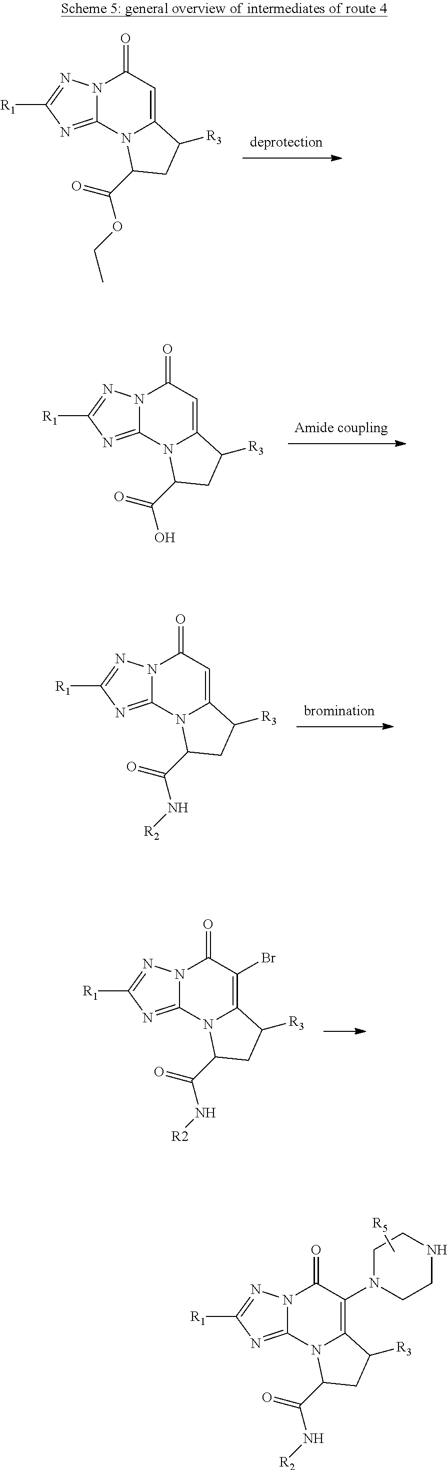

### Intermediate R: rac-5-oxo-2-phenyl-6-(piperazin-1-yl)-N-(4-(trifluoromethyl)phenyl)-5,7,8,9-tetrahydropyrrolo[1,2-c][1,2,4]triazolo[1,5-a]pyrimidine-9-carboxamide

### Step 1: rac-ethyl 4-(7-oxo-2-phenyl-4,7-dihydro-[1,2,4]triazolo[1,5-a]pyrimidin-5-yl)butanoate

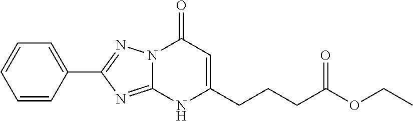

3-Phenyl-1H-1,2,4-triazol-5-amine (4.10 g, 25.6 mmol) was dissolved in EtOH (10 mL). Diethyl 3-oxoheptanedioate (6.30 g, 27.4 mmol) was added. Acetic acid (10.5 g, 175 mmol) was added and the RM was stirred at 110° C. overnight. The RM was concentrated under reduced pressure until a precipitate appeared. The white precipitate was filtered off and washed with EtOH. The precipitate was mixed with EtOH and stirred for a few minutes, then it was concentrated under reduced pressure to give the title compound (4.61 g, 98% pure, yield: 54%).

LC-MS: Rt=0.78 min; MS m/z [M+H]⁺ 327.2, MS m/z [M−H]⁻ 325.1; UPLC-MS 5

### Step 2: rac-ethyl 5-oxo-2-phenyl-5,7,8,9-tetrahydropyrrolo[1,2-c][1,2,4]triazolo[1,5-a]pyrimidine-9-carboxylate

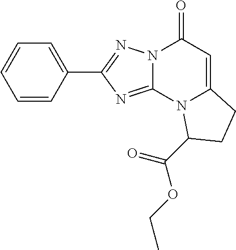 [SMILES: CCOC(=O)C1CCc2cc(=O)n3nc(-c4ccccc4)nc3n21]

Rac-ethyl 4-(7-oxo-2-phenyl-4,7-dihydro-[1,2,4]triazolo[1,5-a]pyrimidin-5-yl)butanoate (2.42 g, 7.27 mmol) was vacuumed and purged with nitrogen. The process was done 3 times, then dry THF (90 mL) was added. The solution was cooled down to −78° C., then LDA 2M (2.72 g, 25.4 mmol) was added dropwise over 9.5 hours. The resulting dark orange mixture was stirred at −78° C. for 2 hours. Bromotrimethylsilane (2.41 g, 15.3 mmol) was added dropwise at −75° C. over 30 minutes. The RM was stirred at −75° C. for 30 minutes. The RM was warmed to RT, then EtOAc was added. The organic layer was washed with aq sat NaHCO₃ and concentrated under reduced pressure. Then it was redissolved in EtOAc and washed twice with aq sat NH₄Cl and once with brine. The organic layer was dried over Na₂SO₄ and concentrated under reduced pressure to give a pale orange foam. The crude product was adsorbed onto Isolute and purified by column chromatography (RediSep Column: Silica 120 g, eluent heptane:EtOAc 70:30 to 0:100). The product containing fractions were combined, concentrated under reduced pressure and dried under HV to give the title compound (548 mg, 99% pure, yield: 23%).

LC-MS: Rt=0.86 min; MS m/z [M+H]⁺ 325.1; UPLC-MS 5

### Step 3: rac-5-oxo-2-phenyl-5,7,8,9-tetrahydropyrrolo[1,2-c][1,2,4]triazolo[1,5-a]pyrimidine-9-carboxylic acid

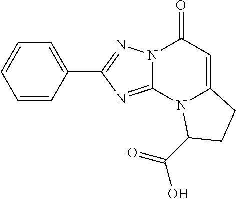 [SMILES: O=C(O)C1CCc2cc(=O)n3nc(-c4ccccc4)nc3n21]

Rac-ethyl 5-oxo-2-phenyl-5,7,8,9-tetrahydropyrrolo[1,2-c][1,2,4]triazolo[1,5-a]pyrimidine-9-carboxylate (545 mg, 1.68 mmol) was dissolved in THF (20 mL). Aq LiOH 376 mM (5.36 mL, 2.01 mmol) was added. The RM was stirred at RT for 3 hours. The RM was concentrated under reduced pressure to give a white powder.

LC-MS: Rt=0.50 min; MS m/z [M+H]⁺ 297.1, MS m/z [M−H]⁻ 295.1; UPLC-MS 3

### Step 4: rac-5-oxo-2-phenyl-N-(4-(trifluoromethyl)phenyl)-5,7,8,9-tetrahydropyrrolo[1,2-c][1,2,4]triazolo[1,5-a]pyrimidine-9-carboxamide

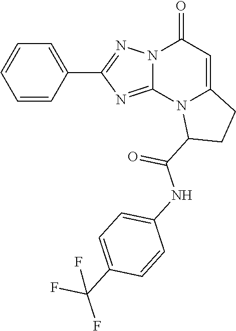 [SMILES: O=C(Nc1ccc(C(F)(F)F)cc1)C1CCc2cc(=O)n3nc(-c4ccccc4)nc3n21]

Rac-5-oxo-2-phenyl-5,7,8,9-tetrahydropyrrolo[1,2-c][1,2,4]triazolo[1,5-a]pyrimidine-9-carboxylic acid was mixed with DCM (15 mL). HBTU (764 mg, 2.02 mmol) was added, followed by BEMP (1.38 g, 5.04 mmol) and 4-(trifluoromethyl)aniline (298 mg, 1.85 mmol). The RM was stirred at RT for 24 hours. HBTU (764 mg, 2.02 mmol), BEMP (460 mg, 1.68 mmol) and 4-(trifluoromethyl)aniline (298 mg, 1.85 mmol) were added again and the RM was stirred at RT overnight. HBTU (764 mg, 2.02 mmol), BEMP (460 mg, 1.68 mmol) and 4-(trifluoromethyl)aniline (298 mg, 1.85 mmol) were added again and the RM was stirred at 45° C. for 2 days. EtOAc was added and the organic layer was washed with aq sat NaHCO₃, twice with water and again with aq sat NaHCO₃, then it was dried through a phase separator and concentrated under reduced pressure. The crude product was purified by reverse phase preparative ISCO (RediSep Column: C18 86 g, eluent water+0.1% TFA:ACN 90:10 to 0:100 in 20 min), then in 2 portions by reverse phase preparative HPLC (RP-HPLC acidic 1: 27 to 57% B in 20 min), (RP-HPLC acidic 1: 27 to 57% B in 20 min). The product containing fractions were combined, basified with aq sat NaHCO₃ and extracted with DCM. The organic layer was dried through a phase separator and concentrated under reduced pressure to give the title compound as a beige powder (183 mg, 96% pure, yield: 24%).

LC-MS: Rt=0.98 min; MS m/z [M+H]⁺ 440.2, MS m/z [M−H]⁻ 438.3; UPLC-MS 3

LC-MS: Rt=5.30 min; MS m/z [M+H]⁺ 440.1, MS m/z [M−H]⁻ 438.0; UPLC-MS 4

### Step 5: rac-6-bromo-5-oxo-2-phenyl-N-(4-(trifluoromethyl)phenyl)-5,7,8,9-tetrahydropyrrolo[1,2-c][1,2,4]triazolo[1,5-a]pyrimidine-9-carboxamide

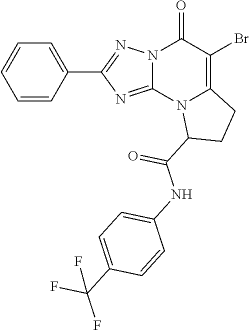 [SMILES: O=C(Nc1ccc(C(F)(F)F)cc1)C1CCc2c(Br)c(=O)n3nc(-c4ccccc4)nc3n21]

Rac-5-oxo-2-phenyl-N-(4-(trifluoromethyl)phenyl)-5,7,8,9-tetrahydropyrrolo[1,2-c][1,2,4]triazolo[1,5-a]pyrimidine-9-carboxamide (180 mg, 409 μmol) was mixed with NBS (76.0 mg, 413 μmol), followed by addition of acetic acid (3 mL). The RM was stirred at 45° C. for 2.75 hours. The white precipitate was filtered, washed with isopropanol and dried under HV to give the title compound as a white powder (186 mg, 82% pure, yield: 72%).

LC-MS: Rt=1.03 min; MS m/z [M+H]⁺ 518.1/520.1, MS m/z [M−H]⁻ 516.2/518.2; UPLC-MS 3

LC-MS: Rt=5.70 min; MS m/z [M+H]⁺ 518.0/520.0, MS m/z [M−H]⁻ 515.9/517.9; UPLC-MS 4

### Step 6: rac-5-oxo-2-phenyl-6-(piperazin-1-yl)-N-(4-(trifluoromethyl)phenyl)-5,7,8,9-tetrahydropyrrolo[1,2-c][1,2,4]triazolo[1,5-a]pyrimidine-9-carboxamide

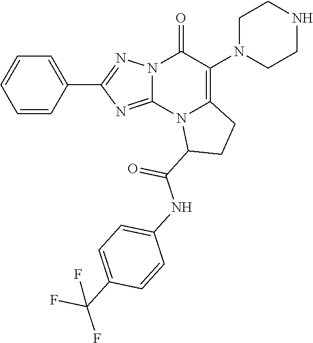 [SMILES: O=C(Nc1ccc(C(F)(F)F)cc1)C1CCc2c(N3CCNCC3)c(=O)n3nc(-c4ccccc4)nc3n21]

Rac-6-bromo-5-oxo-2-phenyl-N-(4-(trifluoromethyl)phenyl)-5,7,8,9-tetrahydropyrrolo[1,2-c][1,2,4]triazolo[1,5-a]pyrimidine-9-carboxamide (169 mg, 261 μmol) and piperazine (2.70 g, 31.3 mmol) were mixed in dry DMSO (3 mL) under N₂ atmosphere and the RM was stirred at 140° C. for 1.5 hours. Piperazine (2.70 g, 31.3 mmol) was added again and the RM was continued stirring at 140° C. for 30 minutes. The RM was cooled to RT. EtOAc was added. The organic layer was washed twice with aq sat NaHCO₃ and with brine. A white precipitate appeared between both phases, which was filtered off. The white solid was dried under HV to give the title compound (89.3 mg, 99% pure, yield: 65%). The filtrate was recovered and both phases were separated. The organic layer was dried through a phase separator and concentrated under reduced pressure. The crude product was purified by reverse phase preparative ISCO (Prep Atlantis T3 OBD 5 μm, 30×150 mm WATERS, eluent water+0.1% TFA:ACN 76:24 to 46:54 in 20 min). The product containing fractions were combined, basified with aq sat NaHCO₃, extracted with DCM, dried through a phase separator and concentrated under reduced pressure to give the title compound as a white powder (20.9 mg, 99% pure, yield: 15%).

Total: 110 mg, 99% pure, yield: 80%

LC-MS: Rt=0.94 min; MS m/z [M+H]⁺ 524.3, MS m/z [M−H]⁻ 522.3; UPLC-MS 3

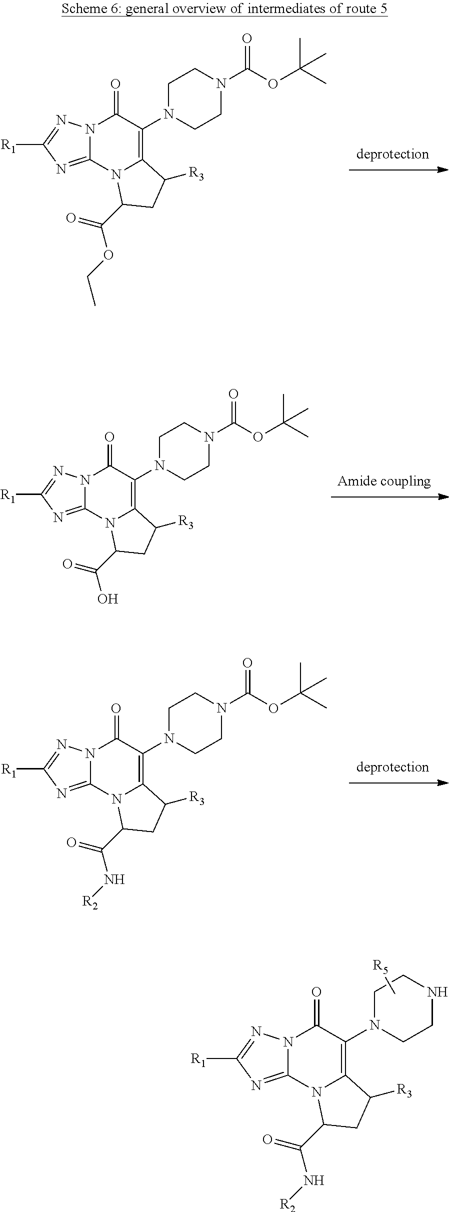

### Intermediate S1: tert-butyl (S)-4-(2-(3,6-dihydro-2H-pyran-4-yl)-5-oxo-9-((4-(trifluoromethyl)phenyl)carbamoyl)-5,7,8,9-tetrahydropyrrolo[1,2-c][1,2,4]triazolo[1,5-a]pyrimidin-6-yl)piperazine-1-carboxylate and Intermediate S2: tert-butyl (R)-4-(2-(3,6-dihydro-2H-pyran-4-yl)-5-oxo-9-((4-(trifluoromethyl)phenyl)carbamoyl)-5,7,8,9-tetrahydropyrrolo[1,2-c][1,2,4]triazolo[1,5-a]pyrimidin-6-yl)piperazine-1-carboxylate

### Step 1: tert-butyl 4-(2-bromo-5-(4-ethoxy-4-oxobutyl)-7-oxo-4-((2-(trimethylsilyl)ethoxy)methyl)-4,7-dihydro-[1,2,4]triazolo[1,5-a]pyrimidin-6-yl)piperazine-1-carboxylate

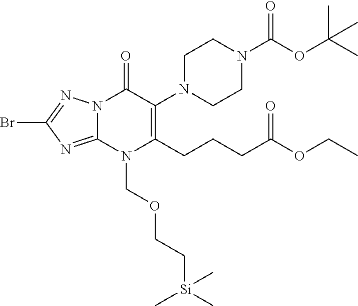 [SMILES: CCOC(=O)CCCc1c(N2CCN(C(=O)OC(C)(C)C)CC2)c(=O)n2nc(Br)nc2n1COCC[Si](C)(C)C]

To a solution of 3-bromo-1H-1,2,4-triazol-5-amine (Intermediate AI) (1.35 g, 7.87 mmol) and diethyl 2-(4-(tert-butoxycarbonyl)piperazin-1-yl)-3-oxoheptanedioate (Intermediate AF) (3.26 g, 7.87 mmol) in EtOH (8.41 mL) was added TFA (909 μL, 11.8 mmol). The RM was stirred at 90° C. for 28 hours. The RM was cooled to RT. Then Et₃N (2.74 mL, 19.7 mmol) and di-tert-butyl dicarbonate (1.83 mL, 7.87 mmol) were added and the RM was stirred at RT for 1 hour. The RM was concentrated under reduced pressure and the residue was dissolved in EtOAc and washed with water. The aqueous layer was extracted several times with EtOAc, dried over a phase separator and concentrated under reduced pressure. The residue was dissolved in DMF (16 mL) and the solution was cooled to 0° C. NaH 60% in mineral oil (378 mg, 9.44 mmol) was added and the RM was stirred for 30 minutes, then (2-(chloromethoxy)ethyl)trimethylsilane (1.68 mL, 9.44 mmol) was added. The RM was stirred at 0° C. for 2 hours. The RM was quenched with water and extracted twice with EtOAc. The combined organic layers were dried through a phase separator and concentrated under reduced pressure. The crude product was purified by column chromatography (RediSep Column: Silica 120 g, eluent cyclohexane:EtOAc 100:0 to 20:80). The product containing fractions were combined, concentrated under reduced pressure and dried under HV to give the title compound as a colourless oil (1.27 g, 92% pure, yield: 25%).

LC-MS: Rt=1.51 min; MS m/z [M+H]⁺ 643.4/645.4; UPLC-MS 6

### Step 2: tert-butyl 4-(2-(3,6-dihydro-2H-pyran-4-yl)-5-(4-ethoxy-4-oxobutyl)-7-oxo-4-((2-(trimethylsilyl)ethoxy)methyl)-4,7-dihydro-[1,2,4]triazolo[1,5-a]pyrimidin-6-yl)piperazine-1-carboxylate

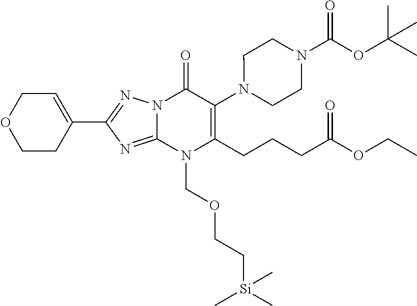 [SMILES: CCOC(=O)CCCc1c(N2CCN(C(=O)OC(C)(C)C)CC2)c(=O)n2nc(C3=CCOCC3)nc2n1COCC[Si](C)(C)C]

Tert-butyl 4-(2-bromo-5-(4-ethoxy-4-oxobutyl)-7-oxo-4-((2-(trimethylsilyl)ethoxy)methyl)-4,7-dihydro-[1,2,4]triazolo[1,5-a]pyrimidin-6-yl)piperazine-1-carboxylate (1.27 g, 1.97 mmol), 2-(3,6-dihydro-2H-pyran-4-yl)-4,4,5,5-tetramethyl-1,3,2-dioxaborolane (622 mg, 2.96 mmol), aq K₃PO₄ (2.63 mL, 3.95 mmol) and XPhos Pd G3 (167 mg, 197 μmol) in 1,4-dioxane (7 mL) was degassed with argon and then stirred at 100° C. for 1 hour. The RM was diluted with EtOAc and washed with water. The organic layer was dried through a phase separator and concentrated under reduced pressure. The crude product was purified by column chromatography (RediSep Column: Silica 24 g, eluent cyclohexane:EtOAc 100:0 to 50:50). The product containing fractions were combined, concentrated under reduced pressure and dried under HV to give the title compound as an orange solid (913 mg, 95% pure, yield: 68%).

LC-MS: Rt=1.45 min; MS m/z [M+H]⁺ 647.6; UPLC-MS 6

### Step 3: tert-butyl 4-(2-(3,6-dihydro-2H-pyran-4-yl)-5-(4-ethoxy-4-oxobutyl)-7-oxo-4,7-dihydro-[1,2,4]triazolo[1,5-a]pyrimidin-6-yl)piperazine-1-carboxylate

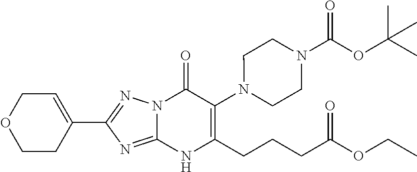 [SMILES: CCOC(=O)CCCc1[nH]c2nc(C3=CCOCC3)nn2c(=O)c1N1CCN(C(=O)OC(C)(C)C)CC1]

To a solution of tert-butyl 4-(2-(3,6-dihydro-2H-pyran-4-yl)-5-(4-ethoxy-4-oxobutyl)-7-oxo-4-((2-(trimethylsilyl)ethoxy)methyl)-4,7-dihydro-[1,2,4]triazolo[1,5-a]pyrimidin-6-yl)piperazine-1-carboxylate (1.05 g, 1.54 mmol) in THF (6 mL) was added TBAF 1M in THF (3.08 mL, 3.08 mmol). The RM was stirred at 50° C. for 5 hours, then at RT overnight. TBAF 1 M in THF (3.08 mL, 3.08 mmol) was added again and the RM was stirred at 50° C. for 6 hours. The RM was cooled to RT and concentrated under reduced pressure. The crude product was purified by column chromatography (RediSep Column: Silica 40 g, eluent cyclohexane:EtOAc 100:0 to 0:100). The product containing fractions were combined, concentrated under reduced pressure and dried under HV to give the title compound as a white solid (315 mg, 90% pure, yield: 36%).

LC-MS: Rt=1.00 min; MS m/z [M+H]⁺ 517.5, MS m/z [M−H]⁻ 515.4; UPLC-MS 6

### Step 4: rac-ethyl 6-(4-(tert-butoxycarbonyl)piperazin-1-yl)-2-(3,6-dihydro-2H-pyran-4-yl)-5-oxo-5,7,8,9-tetrahydropyrrolo[1,2-c][1,2,4]triazolo[1,5-a]pyrimidine-9-carboxylate

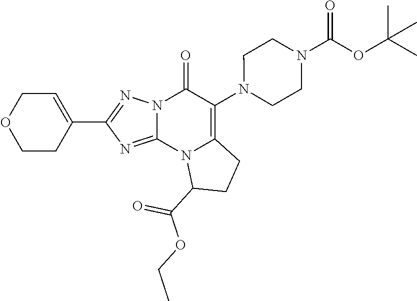 [SMILES: CCOC(=O)C1CCc2c(N3CCN(C(=O)OC(C)(C)C)CC3)c(=O)n3nc(C4=CCOCC4)nc3n21]

A white solution of tert-butyl 4-(2-(3,6-dihydro-2H-pyran-4-yl)-5-(4-ethoxy-4-oxobutyl)-7-oxo-4,7-dihydro-[1,2,4]triazolo[1,5-a]pyrimidin-6-yl)piperazine-1-carboxylate (315 mg, 549 μmol) in THF (3 mL) was cooled to −78° C. Then a solution of LiHMDS 1 M in THF (3.84 mL, 3.84 mmol) was added dropwise and the RM was stirred at −78° C. for 2.5 hours. Then chlorotrimethylsilane (351 μL, 2.74 mmol) was added and the RM was stirred at −78° C. for 30 minutes. NBS (117 mg, 659 μmol) in THF (500 μL) was added at −78° C. and then the RM was stirred at −78° C. for 30 minutes. The RM was quenched with water and extracted twice with EtOAc. The organic layer was dried through a phase separator and concentrated under reduced pressure. The crude product was purified by column chromatography (RediSep Column: Silica 12 g, eluent cyclohexane:EtOAc 100:0 to 0:100). The product containing fractions were combined, concentrated under reduced pressure and dried under HV to give the title compound as a white solid (190 mg, 75% pure, yield: 51%).

LC-MS: Rt=0.94 min; MS m/z [M+H−Boc]⁺ 415.4, m/z [M+H]⁺ 515.4; UPLC-MS 6

### Step 5: rac-6-(4-(tert-butoxycarbonyl)piperazin-1-yl)-2-(3,6-dihydro-2H-pyran-4-yl)-5-oxo-5,7,8,9-tetrahydropyrrolo[1,2-c][1,2,4]triazolo[1,5-a]pyrimidine-9-carboxylic acid

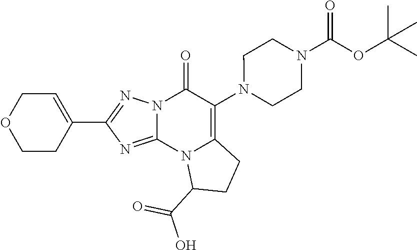 [SMILES: CC(C)(C)OC(=O)N1CCN(c2c3n(c4nc(C5=CCOCC5)nn4c2=O)C(C(=O)O)CC3)CC1]

To a solution of rac-ethyl 6-(4-(tert-butoxycarbonyl)piperazin-1-yl)-2-(3,6-dihydro-2H-pyran-4-yl)-5-oxo-5,7,8,9-tetrahydropyrrolo[1,2-c][1,2,4]triazolo[1,5-a]pyrimidine-9-carboxylate (190 mg, 277 μmol) in THF (2 mL) and water (1 mL) was added LiOH·H₂O (12.8 mg, 305 μmol). The RM was stirred at RT for 4 hours. The solution was diluted with water and extracted with EtOAc. The aqueous layer was acidified with 1N HCl and extracted twice with EtOAc. The combined organic layers were dried through a phase separator and concentrated under reduced pressure to give the title compound as a white solid (156 mg, 85% pure, yield: 98%).

LC-MS: Rt=0.67 min; MS m/z [M−H]⁻ 487.4; UPLC-MS 6

### Step 6: tert-butyl (S)-4-(2-(3,6-dihydro-2H-pyran-4-yl)-5-oxo-9-((4-(trifluoromethyl)phenyl)carbamoyl)-5,7,8,9-tetrahydropyrrolo[1,2-c][1,2,4]triazolo[1,5-a]pyrimidin-6-yl)piperazine-1-carboxylate and tert-butyl (R)-4-(2-(3,6-dihydro-2H-pyran-4-yl)-5-oxo-9-((4-(trifluoromethyl)phenyl)carbamoyl)-5,7,8,9-tetrahydropyrrolo[1,2-c][1,2,4]triazolo[1,5-a]pyrimidin-6-yl)piperazine-1-carboxylate

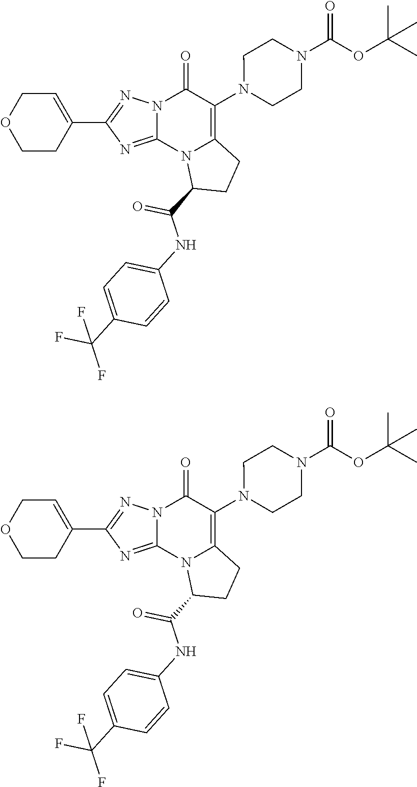 [SMILES: CC(C)(C)OC(=O)N1CCN(c2c3n(c4nc(C5=CCOCC5)nn4c2=O)[C@H](C(=O)Nc2ccc(C(F)(F)F)cc2)CC3)CC1 ; CC(C)(C)OC(=O)N1CCN(c2c3n(c4nc(C5=CCOCC5)nn4c2=O)[C@@H](C(=O)Nc2ccc(C(F)(F)F)cc2)CC3)CC1]

To a solution of rac-6-(4-(tert-butoxycarbonyl)piperazin-1-yl)-2-(3,6-dihydro-2H-pyran-4-yl)-5-oxo-5,7,8,9-tetrahydropyrrolo[1,2-c][1,2,4]triazolo[1,5-a]pyrimidine-9-carboxylic acid (156 mg, 273 μmol) in DCM (4 mL) was added HATU (155 mg, 409 μmol). The RM was stirred for 15 minutes and then 4-(trifluoromethyl)aniline (51.0 μL, 409 μmol) was added, followed by BEMP (197 μL, 681 μmol). The resulting RM was stirred at 40° C. for 16 hours. The RM was diluted with DCM and washed with water. The aqueous layer was extracted with EtOAc. The combined organic layers were dried through a phase separator and concentrated under reduced pressure. The crude product was purified by column chromatography (RediSep Column: Silica 12 g, eluent DCM:MeOH 100:0 to 95:5). The product containing fractions were combined, concentrated under reduced pressure and dried under HV (89.0 mg, 94% pure, yield: 52%).

The racemate (89.0 mg) was purified by preparative chiral HPLC (instrument: Waters Prep SFC100 MS, column: Chiralpak IB-N 250 mm×30 mm 5 μm; eluent: A: 50% 0.1% NH₃ in MeOH, B: 50% scCO₂; flow rate: 80.0 mL/min; detection: UV DAD; injection volume: 4.0 mL; gradient: isocratic A: 50%, B: 50%; oven temperature: 40° C.; BPR: 120 bar).

Peak 1: tert-butyl (S)-4-(2-(3,6-dihydro-2H-pyran-4-yl)-5-oxo-9-((4-(trifluoromethyl)phenyl)carbamoyl)-5,7,8,9-tetrahydropyrrolo[1,2-c][1,2,4]triazolo[1,5-a]pyrimidin-6-yl)piperazine-1-carboxylate (Intermediate S1)

White solid, 36.0 mg, 97% pure, yield: 40%

LC-MS: Rt=1.26 min; MS m/z [M+H−Boc]⁺ 530.3, m/z [M−H]⁻ 628.6; UPLC-MS 6

Chiral HPLC (C-HPLC 13): Rt=0.61 min, 99% ee

Peak 2: tert-butyl (R)-4-(2-(3,6-dihydro-2H-pyran-4-yl)-5-oxo-9-((4-(trifluoromethyl)phenyl)carbamoyl)-5,7,8,9-tetrahydropyrrolo[1,2-c][1,2,4]triazolo[1,5-a]pyrimidin-6-yl)piperazine-1-carboxylate (Intermediate S2)

White solid, 33.0 mg, 100% pure, yield: 37%

LC-MS: Rt=1.26 min; MS m/z [M+H−Boc]⁺ 530.3, m/z [M−H]⁻ 628.6; UPLC-MS 6

Chiral HPLC (C-HPLC 13): Rt=2.82 min, 99% ee

### Intermediate T1: 2-(3,6-dihydro-2H-pyran-4-yl)-5-oxo-6-(piperazin-1-yl)-N-(4-(trifluoromethyl)phenyl)-5,7,8,9-tetrahydropyrrolo[1,2-c][1,2,4]triazolo[1,5-a]pyrimidine-9-carboxamide

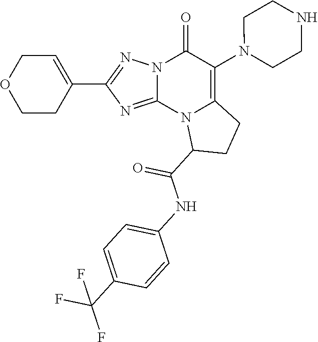 [SMILES: O=C(Nc1ccc(C(F)(F)F)cc1)C1CCc2c(N3CCNCC3)c(=O)n3nc(C4=CCOCC4)nc3n21]

A solution of rac-tert-butyl 4-(2-(3,6-dihydro-2H-pyran-4-yl)-5-oxo-9-((4-(trifluoromethyl)phenyl)carbamoyl)-5,7,8,9-tetrahydropyrrolo[1,2-c][1,2,4]triazolo[1,5-a]pyrimidin-6-yl)piperazine-1-carboxylate (Intermediate S—racemate) (16.0 mg, 25.0 μmol) in 4M HCl in 1,4-dioxane (95.0 μL, 381 μmol) was stirred at RT for 1 hour. The solution was concentrated under reduced pressure and dried under vacuum to give the title compound as a white solid (16.0 mg, 95% pure, yield: 100%).

LC-MS: Rt=0.68 min; MS m/z [M+H]⁺ 530.4, m/z [M−H]⁻ 528.3; UPLC-MS 6

### Intermediate T2: (S)-2-(3,6-dihydro-2H-pyran-4-yl)-5-oxo-6-(piperazin-1-yl)-N-(4-(trifluoromethyl)phenyl)-5,7,8,9-tetrahydropyrrolo[1,2-c][1,2,4]triazolo[1,5-a]pyrimidine-9-carboxamide

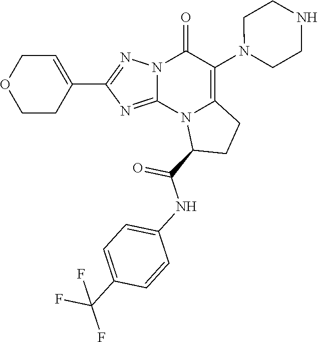 [SMILES: O=C(Nc1ccc(C(F)(F)F)cc1)[C@@H]1CCc2c(N3CCNCC3)c(=O)n3nc(C4=CCOCC4)nc3n21]

A solution of tert-butyl (S)-4-(2-(3,6-dihydro-2H-pyran-4-yl)-5-oxo-9-((4-(trifluoromethyl)phenyl)carbamoyl)-5,7,8,9-tetrahydropyrrolo[1,2-c][1,2,4]triazolo[1,5-a]pyrimidin-6-yl)piperazine-1-carboxylate (Intermediate S1) (36.0 mg, 57.0 μmol) in 4M HCl in 1,4-dioxane (214 μL, 858 μmol) was stirred at RT for 4 hours. The solution was concentrated under reduced pressure and the solid was dried under vacuum to give the title compound as a slightly yellow solid (34.0 mg, 95% pure, yield: 100%).

LC-MS: Rt=0.70 min; MS m/z [M+H]⁺ 530.3, m/z [M−H]⁻ 528.5; UPLC-MS 6

### Intermediate T3: (R)-2-(3,6-dihydro-2H-pyran-4-yl)-5-oxo-6-(piperazin-1-yl)-N-(4-(trifluoromethyl)phenyl)-5,7,8,9-tetrahydropyrrolo[1,2-c][1,2,4]triazolo[1,5-a]pyrimidine-9-carboxamide

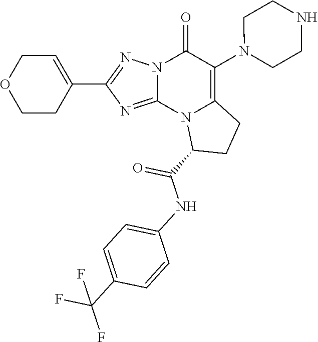 [SMILES: O=C(Nc1ccc(C(F)(F)F)cc1)[C@H]1CCc2c(N3CCNCC3)c(=O)n3nc(C4=CCOCC4)nc3n21]

A solution of tert-butyl (R)-4-(2-(3,6-dihydro-2H-pyran-4-yl)-5-oxo-9-((4-(trifluoromethyl)phenyl)carbamoyl)-5,7,8,9-tetrahydropyrrolo[1,2-c][1,2,4]triazolo[1,5-a]pyrimidin-6-yl)piperazine-1-carboxylate (Intermediate S2) (33.0 mg, 52.0 μmol) in 4M HCl in 1,4-dioxane (197 μL, 786 μmol) was stirred at RT for 1 hour. The solution was concentrated under reduced pressure and the solid was dried under vacuum to give the title compound as a white solid (29.0 mg, 100% pure, yield: 98%).

LC-MS: Rt=0.72 min; MS m/z [M+H]⁺ 530.3, m/z [M−H]⁻ 528.5; UPLC-MS 6

### Preparation of Early Intermediates

### Intermediate U: 5-(benzyloxy)-6-methylpyrimidine-4-carboxylic acid

### Step 1: 5-(benzyloxy)-4,6-dichloropyrimidine

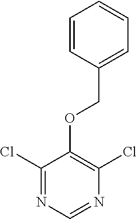 [SMILES: Clc1ncnc(Cl)c1OCc1ccccc1]

To a stirred solution of 4,6-dichloropyrimidin-5-ol (29.5 g, 179 mmol) in DMF (100 mL) was added K₂CO₃ (32.1 g, 232 mmol) then (bromomethyl)benzene (23.4 mL, 197 mmol) at RT and the RM was stirred at RT for 3 hours. The RM was diluted with EtOAc and water, extracted once with EtOAc and the organic layer was washed with brine, dried over Na₂SO₄ and concentrated under reduced pressure. The crude product was purified by column chromatography (RediSep Column: Silica 330 g, eluent heptane:EtOAc 100:0 to 87:13). The product containing fractions were combined and concentrated under reduced pressure to give the title compound as a white solid (45.1 g, 100% pure, yield: 99%).

LC-MS: Rt=1.18 min; no mass observed; UPLC-MS 1

### Step 2: 5-(benzyloxy)-4-chloro-6-methylpyrimidine

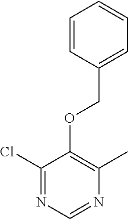 [SMILES: Cc1ncnc(Cl)c1OCc1ccccc1]

To a stirred solution of 5-(benzyloxy)-4,6-dichloropyrimidine (32.5 g, 127 mmol), K₃PO₄ (81.0 g, 382 mmol) and PdCl₂(dppf)·DCM (5.20 g, 6.36 mmol) in toluene (350 mL) and water (100 mL) was added methyl boronic acid (9.17 g, 153 mmol) in 1,4-dioxane (45 mL) at 105° C. and the reaction was stirred at 105° C. for 18 hours. Methyl boronic acid (9.17 g, 153 mmol) was added again and the RM was stirred at 105° C. for 8 hours. Methyl boronic acid (9.17 g, 153 mmol) was added again and the RM was stirred at 105° C. for 4 hours. The RM was diluted with EtOAc and water, extracted twice with EtOAc and the combined organic layers were washed with water and brine, dried over Na₂SO₄ and concentrated under reduced pressure. The crude product was purified by column chromatography (RediSep Column: Silica 330 g, eluent heptane:EtOAc 100:0 to 77:23). The product containing fractions were combined and concentrated under reduced pressure to give the title compound as a yellow oil (21.4 g, 100% pure, yield: 72%).

LC-MS: Rt=1.03 min; MS m/z [M+H]⁺ 235.2/237.2; UPLC-MS 1

### Step 3: methyl 5-(benzyloxy)-6-methylpyrimidine-4-carboxylate

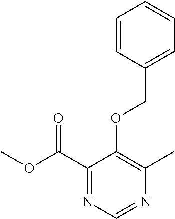 [SMILES: COC(=O)c1ncnc(C)c1OCc1ccccc1]

A solution of 5-(benzyloxy)-4-chloro-6-methylpyrimidine (25.9 g, 110 mmol), PdCl₂(dppf)·DCM (4.51 g, 5.52 mmol) and Et₃N (30.8 mL, 221 mmol) in MeOH (25 mL) was stirred at 50° C. under 10 bar of CO for 40 hours. The RM was filtered through celite and concentrated under reduced pressure. The residue was triturated with DCM and the solid was filtered off. The filtrate was purified by column chromatography (eluent heptane:EtOAc 100:0 to 60:40). The product containing fractions were combined and concentrated under reduced pressure to give the title compound as a white solid (13.0 g, 100% pure, yield: 46%).

LC-MS: Rt=0.81 min; MS m/z [M+H]⁺ 259.1; UPLC-MS 1

### Step 4: 5-(benzyloxy)-6-methylpyrimidine-4-carboxylic acid

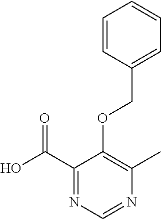 [SMILES: Cc1ncnc(C(=O)O)c1OCc1ccccc1]

To a stirred solution of methyl 5-(benzyloxy)-6-methylpyrimidine-4-carboxylate (22.9 g, 88.0 mmol) in THF (100 mL) and MeOH (100 mL) was added NaOH 2N in water (100 mL, 200 mmol) at RT and the RM was stirred at RT for 5 minutes. THF and MeOH were removed under reduced pressure, then the resulting aqueous residue was acidified to pH 3 with 2N HCl and the mixture was filtered to give the title compound as a white solid (21.6 g, 100% pure, yield: 100%).

LC-MS: Rt=0.43 min; MS m/z [M+H]⁺ 245.2, m/z [M−H]⁻ 243.1; UPLC-MS 1

### Intermediate V: 3-(benzyloxy)-4-fluoro-6-methylpicolinic acid

### Step 1: 2-bromo-6-methyl-4-nitropyridin-3-ol

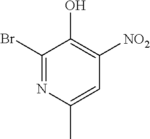

2-Bromo-6-methylpyridin-3-ol (15.0 g, 80.0 mmol) was mixed with H₂SO₄ (44.8 mL, 798 mmol) at 0° C. HNO₃ (66%) (6.03 mL, 88.0 mmol) was added dropwise within 12 minutes. The orange solution was stirred at 0° C. for 1 hour, then at RT for 3 hours. HNO₃ (66%) (1.10 mL, 16.0 mmol) was added and the reaction was stirred at RT for 3 hours. The RM was poured while vigorous stirring onto ice. The mixture was extracted with EtOAc (3×600 mL). The organic layer was washed with water (2×75 mL) and brine (2×75 mL), dried over Na₂SO₄, filtered and concentrated under reduced pressure. The residue was adsorbed onto Isolute and purified by column chromatography (RediSep Column: Silica 40 g, eluent DCM:DCM/MeOH (8/2) 100:0 to 0:100). The product containing fractions were combined and concentrated under reduced pressure to give the title compound as a yellow solid (10.7 g, 88% pure, yield: 51%).

LC-MS: Rt=0.53 min; MS m/z [M−H]⁻ 231.0/233.0; LC-MS 1

### Step 2: 3-(benzyloxy)-2-bromo-6-methyl-4-nitropyridine

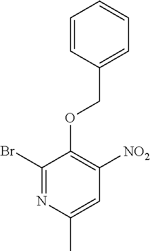 [SMILES: Cc1cc([N+](=O)[O-])c(OCc2ccccc2)c(Br)n1]

2-Bromo-6-methyl-4-nitropyridin-3-ol (10.4 g, 44.5 mmol) and K₂CO₃ (18.5 g, 134 mmol) were dissolved in DMF (110 mL) at RT under argon. (Bromomethyl)benzene (11.7 g, 66.8 mmol) was added and the RM was stirred at 45° C. for 17 hours. The RM was cooled to RT, filtered and the cake was washed with ACN. The filtrate was concentrated under reduced pressure, the residue was adsorbed onto Isolute and purified by column chromatography (RediSep Column: Silica 220 g, eluent DCM). The product containing fractions were combined and concentrated under reduced pressure to give the title compound as an orange oil (9.30 g, 97% pure, yield: 63%).

LC-MS: Rt=1.25 min; MS m/z [M+H]⁺ 323.1/325.1; LC-MS 1

### Step 3: 3-(benzyloxy)-2-bromo-4-fluoro-6-methylpyridine

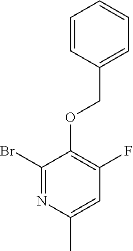 [SMILES: Cc1cc(F)c(OCc2ccccc2)c(Br)n1]

3-(Benzyloxy)-2-bromo-6-methyl-4-nitropyridine (9.30 g, 28.8 mmol) was dissolved in DMF (150 mL). The flask was vacuumed and purged with argon several times. TBAF 1M in THF (58.0 mL, 58.0 mmol) was added and the RM was stirred at RT for 45 minutes. TBAF 1M in THF (20.0 mL, 20.0 mmol) was added and the RM was stirred at RT for 1.75 hours. The RM was quenched with water (250 mL) and extracted with EtOAc (3×400 mL). The organic layer was washed with water (200 mL) and brine (2×150 mL), dried over Na₂SO₄ and filtered. The filtrate was concentrated under reduced pressure. The crude product was purified by column chromatography (RediSep Column: Silica 220 g, eluent DCM). The product containing fractions were combined and concentrated under reduced pressure. The residue was purified by column chromatography (RediSep Column: Silica 120 g, eluent DCM). The product containing fractions were combined and concentrated under reduced pressure to give the title compound as a beige solid (4.93 g, 99% pure, yield: 57%).

LC-MS: Rt=1.23 min; MS m/z [M+H]⁺ 296.1/298.1; LC-MS 1

### Step 4: ethyl 3-(benzyloxy)-4-fluoro-6-methylpicolinate

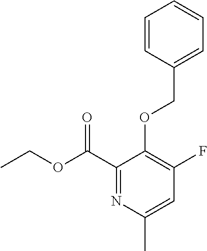 [SMILES: CCOC(=O)c1nc(C)cc(F)c1OCc1ccccc1]

A solution of 3-(benzyloxy)-2-bromo-4-fluoro-6-methylpyridine (4.93 g, 16.7 mmol), PdCl₂(dppf)·DCM (680 mg, 832 μmol) and Et₃N (7.03 mL, 49.9 mmol) in EtOH (520 mL) was stirred at 80° C. under 18 bar of CO for 20 hours. SiliaMetS®Thiol was added, the mixture was stirred at 40° C. for 1 hour, filtered and the cake was washed with DCM. The filtrate was concentrated under reduced pressure, the crude product was suspended in DCM, filtered, washed with Et₂O and purified by column chromatography (RediSep Column: Silica 120 g, eluent DCM:DCM/MeOH (1/1) 100:0 to 0:100). The product containing fractions were combined and concentrated under reduced pressure and further purified by reverse phase preparative HPLC (RP-HPLC acidic 1: 5 to 95% B in 20 min). The product containing fractions were combined, basified with aq sat NaHCO₃, extracted twice with DCM, dried through a phase separator and concentrated under reduced pressure to give the title compound (2.05 g, 84% pure, yield: 36%).

LC-MS: Rt=1.06 min; MS m/z [M+H]⁺ 290.3; LC-MS 1

### Step 5: 3-(benzyloxy)-4-fluoro-6-methylpicolinic acid

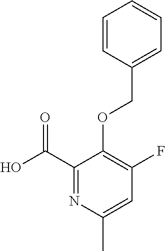 [SMILES: Cc1cc(F)c(OCc2ccccc2)c(C(=O)O)n1]

Ethyl 3-(benzyloxy)-4-fluoro-6-methylpicolinate (2.05 g, 5.95 mmol) was dissolved in 1,4-dioxane (5 mL) and NaOH 1M in water (12.0 mL, 12.0 mmol) was added. The RM was stirred at 50° C. for 45 minutes. The RM was concentrated under reduced pressure and the residue was mixed with water (30 mL), aq 1M HCl (25 mL) and DCM (30 mL). The aqueous layer was washed with DCM (3×20 mL). The combined organic layers were dried through a phase separator and concentrated under reduced pressure to give the title compound (1.91 g, 88% pure, quantitative).

LC-MS: Rt=0.56 min; MS m/z [M+H]⁺ 262.2, m/z [M−H]⁻ 260.1; UPLC-MS 1

### Intermediate W: 3-(benzyloxy)-4-fluoropicolinic acid

### Step 1: 2-chloro-4-fluoro-3-((4-methoxybenzyl)oxy)pyridine

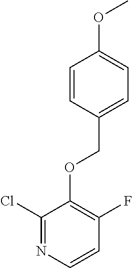 [SMILES: COc1ccc(COc2c(F)ccnc2Cl)cc1]

2-Chloro-4-fluoropyridin-3-ol (3.00 g, 19.3 mml) and K₂CO₃ (4.00 g, 29.0 mmol) were dissolved in DMF (55 mL). The RM was flushed with argon, 1-(chloromethyl)-4-methoxybenzene (2.15 mL, 21.3 mmol) was added and the RM was stirred at 60° C. for 4 hours. EtOAc (100 mL) was added and it was washed with water (2×50 mL) and brine (50 mL), dried over Na₂SO₄, filtered and evaporated. The crude product was purified by column chromatography (RediSep Column: Silica 80 g, eluent heptane/EtOAc 100:0 to 75:25). The product containing fractions were combined, concentrated under reduced pressure and dried under HV to give the title compound (3.28 g, 93% pure, yield: 59%).

LC-MS: Rt=1.08 min; MS m/z [M+H]⁺ 268.0/270.0; UPLC-MS 1

### Step 2: methyl 4-fluoro-3-hydroxypicolinate

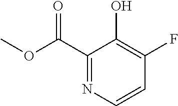

To a stirred and degassed solution of 2-chloro-4-fluoro-3-((4-methoxybenzyl)oxy)pyridine (3.44 g, 12.8 mmol) in Et₃N (3.58 mL, 25.7 mmol) and MeOH (50 mL) was added PdCl₂(dppf)·DCM (524 mg, 642 μmol) and the RM was stirred at 50° C. under 10 bars of CO until completion. The RM was filtered through celite and concentrated under reduced pressure. The crude product was purified by column chromatography (RediSep Column: Silica 40 g, eluent heptane:EtOAc 100:0 to 0:100). The product containing fractions were combined and concentrated under reduced pressure to give the title compound as a white solid (1.52 g, 100% pure, yield: 75%).

LC-MS: Rt=0.37 min; MS m/z [M+H]⁺ 172.1; UPLC-MS 1

### Step 3: methyl 3-(benzyloxy)-4-fluoropicolinate

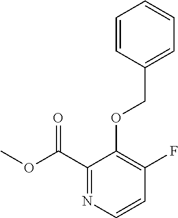 [SMILES: COC(=O)c1nccc(F)c1OCc1ccccc1]

To a stirred solution of methyl 4-fluoro-3-hydroxypicolinate (500 mg, 2.92 mmol) in DMF (5 mL) were added K₂CO₃ (606 mg, 4.38 mmol) and (bromomethyl)benzene (382 μL, 3.21 mmol) and reactants were stirred at RT for 14 hours. The RM was diluted with EtOAc and water, extracted with EtOAc and the organic layer was washed with water and brine, dried over Na₂SO₄ and concentrated under reduced pressure. The crude product was purified by column chromatography (RediSep Column: Silica 24 g, eluent heptane:EtOAc 100:0 to 50:50). The product containing fractions were combined and concentrated under reduced pressure to afford the title compound as a yellow oil (750 mg, 100% pure, yield: 98%).

LC-MS: Rt=0.89 min; MS m/z [M+H]⁺ 262.0; UPLC-MS 1

### Step 4: 3-(benzyloxy)-4-fluoropicolinic acid

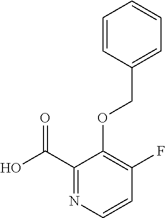 [SMILES: O=C(O)c1nccc(F)c1OCc1ccccc1]

To a stirred solution of methyl 3-(benzyloxy)-4-fluoropicolinate (750 mg, 2.87 mmol) in THF (5 mL) and MeOH (5 mL) was added NaOH 2N in water (5.00 mL, 10.0 mmol) at RT and the RM was stirred at RT for 30 minutes. THF and MeOH were removed under reduced pressure, then the resulting aqueous residue was acidified to pH 3 with 2N HCl and extracted three times with DCM. The combined organic layers were dried over Na₂SO₄ and concentrated under reduced pressure to give the title compound as a white solid (680 mg, 100% pure, yield: 96%).

LC-MS: Rt=0.58 min; MS m/z [M+H]⁺ 248.1, m/z [M−H]⁻ 246.1; UPLC-MS 1

### Intermediate X: 3-hydroxypicolinoyl chloride

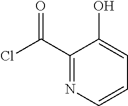

3-Hydroxypicolinic acid (12.0 g, 82.0 mmol) and sulfurous dichloride (121 mL, 1.64 mol) were mixed under argon at RT. The flask was equipped with a condenser and a drying tube (CaCl₂) and the suspension was stirred at RT for 5 days. Then it was filtered and the cake was washed several times with TBME. The solid was dried at 30° C. under HV and stored under argon (13.9 g, 92% pure, yield: 99%).

### Intermediate Y: 4-chloro-3-hydroxypicolinoyl chloride

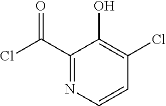 [SMILES: O=C(Cl)c1nccc(Cl)c1O]

A suspension of 4-chloro-3-hydroxypicolinic acid (100 mg, 576 μmol) in thionyl chloride (1.00 mL, 13.7 mmol) was stirred at RT for 6 days. The RM was azeotropically evaporated under vacuum with toluene. The solid was dried under HV overnight to give the title compound as a yellow solid.

### Intermediate Z: 7-(methoxymethoxy)-2,3-dihydrofuro[3,2-c]pyridine-6-carboxylic acid

### Step 1: 6-bromofuro[3,2-c]pyridin-7-ol

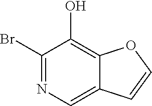 [SMILES: Oc1c(Br)ncc2ccoc12]

The flask was flame-dried in vacuo and backfilled with argon. The dry argon flushed flask was charged with 6-bromo-7-methoxyfuro[3,2-c]pyridine (5.00 g, 20.8 mmol) and anhydrous DCM (69.4 mL). The RM was cooled to −78° C. and then 1M BBr₃ in DCM (125 mL, 125 mmol) was added dropwise. After the complete addition the resulting yellow suspension was warmed to RT and stirred for 13.5 hours. The RM was cooled to −78° C. and then anhydrous MeOH (17.0 mL, 417 mmol) was added dropwise. The reaction was concentrated to dryness. MeOH (10 mL) was added followed by Et₂O (150 mL). The brown suspension was sonicated and filtered. The solid was washed with Et₂O (2×100 mL) and dried at 40° C. under vacuum overnight to give the title compound as a white solid (4.58 g, 98% pure, yield: 73%).

LC-MS: Rt=0.36 min; MS m/z [M+H]⁺ 214.0/216.0, m/z [M−H]⁻ 212.1/214.0; UPLC-MS 1

### Step 2: ethyl 7-hydroxyfuro[3,2-c]pyridine-6-carboxylate

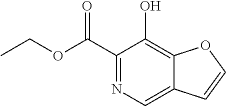

The reactor was charged with 6-bromofuro[3,2-c]pyridin-7-ol (4.58 g, 15.3 mmol), PdCl₂(dppf)·DCM (624 mg, 764 μmol), Et₃N (8.60 mL, 61.1 mmol) and anhydrous EtOH (50 mL). The autoclave was subjected to three cycles of evacuation-backfilling with argon. Subsequently it was filled with 10 bar of CO at RT and then heated at 80° C. for 24 hours. ISOLUTE® Si-TMT (15.6 g, 7.64 mmol) was introduced and the suspension was stirred at 40° C. for 1 hour. The mixture was filtered over a pad of celite. The filtrate was concentrated under reduced pressure. The residue was adsorbed onto Isolute and purified by normal phase chromatography (Buchi® FlashPure ID HP silica cartridge 120 g, eluent heptane:DCM/MeOH (8:2) 100:0 to 20:80). The product containing fractions were combined and concentrated under reduced pressure to give the title compound as a brown solid (2.06 g, 98% pure, yield: 64%).

LC-MS: Rt=0.54 min; MS m/z [M+H]⁺ 208.2; UPLC-MS 1

### Step 3: ethyl 7-hydroxy-2,3-dihydrofuro[3,2-c]pyridine-6-carboxylate

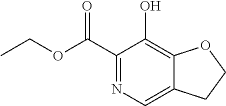

The reactor was charged with Pd—C 10 wt % (518 mg, 487 μmol) and a solution of ethyl 7-hydroxyfuro[3,2-c]pyridine-6-carboxylate (2.06 g, 9.74 mmol) in anhydrous EtOH (32.5 mL). The autoclave was subjected to three cycles of evacuation-backfilling with nitrogen. Subsequently it was filled with 5 bar of hydrogen (20.0 mg, 9.74 mmol) at RT and stirred for 5 days. The RM was filtered over a pad of celite. The cake was washed with EtOH (25 mL). The filtrate was concentrated under reduced pressure. The residue was adsorbed onto Isolute and purified by normal phase chromatography (Buchi® FlashPure ID HP silica cartridge 80 g, eluent DCM:DCM/MeOH (8:2) 100:0 to 70:30). The product containing fractions were combined and concentrated under reduced pressure to give the title compound as a white powder (1.37 g, 98% pure, yield: 66%).

LC-MS: Rt=0.38 min; MS m/z [M+H]⁺ 210.2; UPLC-MS 1

### Step 4: ethyl 7-(methoxymethoxy)-2,3-dihydrofuro[3,2-c]pyridine-6-carboxylate

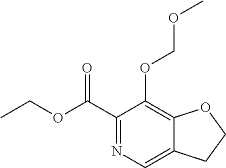 [SMILES: CCOC(=O)c1ncc2c(c1OCOC)OCC2]

To a colourless solution of ethyl 7-hydroxy-2,3-dihydrofuro[3,2-c]pyridine-6-carboxylate (1.12 g, 5.23 mmol) and DMAP (130 mg, 1.05 mmol) in anhydrous DCM (52.3 mL), previously purged/vacuumed with argon was added with DIPEA (1.38 mL, 7.84 mmol). The colourless mixture was cooled down to 0° C. then MOMCl (662 μL, 7.84 mmol) was added dropwise (the colourless solution turned progressively into a dark orange solution). The dark orange RM was stirred at 0° C. for 1.5 hours, the ice bath was removed and the resulting RM was stirred at RT for 3.25 hours. The RM was quenched with aqueous NaHCO₃ (100 mL). The aqueous layer was extracted twice with DCM. The combined organic layers were dried through a phase separator, concentrated and dried under high vacuum. The residue was adsorbed onto Isolute and purified by normal phase chromatography (RediSep Column: Silica 40 g, eluent heptane:EtOAc 100:0 to 10:90). The product containing fractions were combined and concentrated to give the title compound as a white solid (1.19 g, 73% pure, yield: 66%).

LC-MS: Rt=0.48 min; MS m/z [M+H]⁺ 254.2; UPLC-MS 1

### Step 5: 7-(methoxymethoxy)-2,3-dihydrofuro[3,2-c]pyridine-6-carboxylic acid

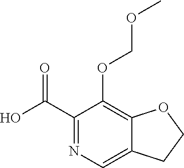 [SMILES: COCOc1c(C(=O)O)ncc2c1OCC2]

To a colourless solution of ethyl 7-(methoxymethoxy)-2,3-dihydrofuro[3,2-c]pyridine-6-carboxylate (1.19 g, 3.42 mmol) in ACN (46 mL) was added 1M NaOH in water (4.60 mL, 4.60 mmol). The resulting colourless RM was stirred at RT for 73 hours. The white suspension was filtered. The cake was washed with a bit of water and dried under vacuum. The filtrate was freezed and lyophilized. The resulting white solid was dissolved in water and extracted twice with DCM. The combined organic layers were dried trough a phase separator, concentrated and dried under vacuum to give the title compound as a sodium salt (871 mg, 95% pure, yield: 97%).

LC-MS: Rt=0.25 min; MS m/z [M+H]⁺ 226.3; UPLC-MS 10

### Intermediate AA: 6-(benzyloxy)imidazo[1,2-a]pyridine-5-carboxylic acid

### Step 1: 5-bromoimidazo[1,2-a]pyridin-6-ol

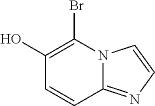 [SMILES: Oc1ccc2nccn2c1Br]

The flask was flame-dried in vacuo and backfilled with argon. The dry argon flushed flask was charged with 5-bromo-6-methoxyimidazo[1,2-a]pyridine (1.00 g, 4.18 mmol) and anhydrous DCM (14 mL). The resulting mixture was cooled to −78° C. and then 1M BBr₃ in DCM (20.9 mL, 20.9 mmol) was added dropwise. After the complete addition the resulting deep brown suspension was warmed to RT. The RM was stirred at RT for 18 hours. The RM was cooled to −78° C. and then anhydrous MeOH (3.42 mL, 84.0 mmol) was added slowly. The RM was concentrated to dryness. MeOH (5 mL) was added followed by Et₂O (100 mL). The brown suspension was sonicated and the solid was filtered off. The solid was washed with Et₂O (2×50 mL) and dried under vacuum to give the title compound as a pink powder (1.19 g, 98% pure, yield: 95%).

LC-MS: Rt=0.23 min; MS m/z [M+H]⁺ 213.2/215.2, MS m/z [M−H]⁻ 210.9/212.9; UPLC-MS 7

### Step 2: 6-(benzyloxy)-5-bromoimidazo[1,2-a]pyridine

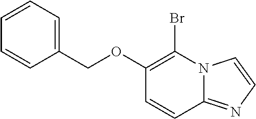 [SMILES: Brc1c(OCc2ccccc2)ccc2nccn12]

The flask was flame-dried in vacuo and backfilled with argon. The dry argon flushed flask was charged with 5-bromoimidazo[1,2-a]pyridin-6-ol (1.19 g, 3.97 mmol) and potassium carbonate (1.66 g, 11.9 mmol). The contents were suspended in anhydrous DMF (9.92 mL) and the resulting mixture was treated with (bromomethyl)benzene (626 μL, 5.16 mmol). The RM was stirred at RT for 21 hours. The reaction was partitioned between water (35 mL) and EtOAc (30 mL). The organic layer was collected and the aqueous layer was back-extracted with EtOAc (3×25 mL). The organic layers were combined, washed with brine (50 mL) and dried through a phase separator. The solvent was reduced to dryness to provide a brownish oil (668 mg). The crude product was purified by normal phase chromatography (Buchi® FlashPure ID HP silica cartridge 24 g, eluent heptane:EtOAc 100:0 to 15:85). The product containing fractions were combined and concentrated to give the title compound as a beige powder (94.6 mg, 98% pure, yield: 8%).

LC-MS: Rt=0.80 min; MS m/z [M+H]⁺ 303.1/305.1; UPLC-MS 1

### Step 3: ethyl 6-(benzyloxy)imidazo[1,2-a]pyridine-5-carboxylate

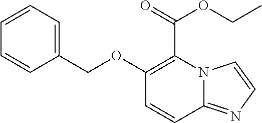 [SMILES: CCOC(=O)c1c(OCc2ccccc2)ccc2nccn12]

The reactor was charged with 6-(benzyloxy)-5-bromoimidazo[1,2-a]pyridine (94.0 mg, 304 μmol), PdCl₂(dppf)·DCM adduct (12.4 mg, 15.0 μmol), Et₃N (128 μL, 912 μmol) and anhydrous EtOH (10 mL). The autoclave was subjected to three cycles of evacuation-backfilling with argon. Subsequently it was filled with 20 bars of CO at RT and then heated at 80° C. for 20 hours. PdCl₂(dppf)·DCM adduct (12.4 mg, 15.0 μmol) and Et₃N (128 μL, 912 μmol) were introduced and the RM was heated at 90° C. for 20 hours. ISOLUTE® Si-TMT (62.0 mg, 30.0 μmol) was introduced to the mixture and it was left to stir at RT. The mixture was filtered off over a pad of celite. Removal of volatiles under pressure to give a yellow-brown solid (175 mg). A dry argon flushed vial was charged with the yellow-brown solid (175 mg) from the previous step and K₂CO₃ (42.4 mg, 304 μmol). The contents were suspended with anhydrous DMF (2 mL) and (bromomethyl)benzene (26.0 μL, 213 μmol) was introduced. The vial was sealed and the resulting mixture was left to react at RT for 16 hours. (Bromomethyl)benzene (13.0 μL, 106 μmol) was introduced and the RM was stirred at RT for 22.5 hours. The reaction was partitioned between water (10 mL) and EtOAc (5 mL). The organic layer was collected and the aqueous layer was back-extracted with EtOAc (3×5 mL). The organic layers were combined, washed with brine (20 mL) and dried through a phase separator. The solvent was reduced to dryness to give a brownish oil (107 mg). The crude product was adsorbed onto Isolute and purified by normal phase chromatography (Buchi® FlashPure ID HP silica cartridge 12 g, eluent heptane:EtOAc 100:0 to 0:100). The product containing fractions were combined and concentrated to give the title compound as a beige solid (37.3 mg, 98% pure, yield: 41%).

LC-MS: Rt=0.79 min; MS m/z [M+H]⁺ 297.4; UPLC-MS 1

### Step 4: 6-(benzyloxy)imidazo[1,2-a]pyridine-5-carboxylic acid

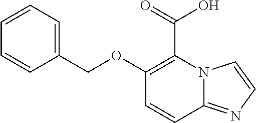 [SMILES: O=C(O)c1c(OCc2ccccc2)ccc2nccn12]

Ethyl 6-(benzyloxy)imidazo[1,2-a]pyridine-5-carboxylate (36.0 mg, 119 μmol) was dissolved in ACN (1.19 mL) and the mixture was treated with 1M NaOH in water (119 μL, 119 μmol). The resulting mixture was stirred at RT for 23.5 hours. 1M NaOH in water (11.9 mL, 119 μmol) was added and the RM was stirred at RT for 21.5 hours. The RM was frozen in a mixture of dry ice/acetone and lyophilized to give the title compound as a white powder (33.7 mg, 98% pure, yield: 95%).

LC-MS: Rt=0.56 min; MS m/z [M+H]⁺ 269.3, MS m/z [M−H]⁻ 267.1; UPLC-MS 10

### Intermediate AB: 3,6-dihydro-2H-pyran-4-carbaldehyde

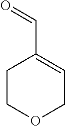

According to Ref. Org. Lett. 2014, 16, 4142-4145.

To a mixture of tetrahydro-4H-pyran-4-one (30.0 g, 300 mmol) and water (300 mL), was added NaCN (15.4 g, 315 mmol) at 5° C. followed by NaHSO₄ until a pH=4-5 was reached. The reaction was stirred at 10° C. for 1 hour, then NaCl (17.5 g, 300 mmol) was added at 25° C. followed by 2-MeTHF. The organic layers were separated and the aqueous layer was extracted twice with 2-MeTHF. The combined organic layers were washed with brine, dried over Na₂SO₄, concentrated under reduced pressure and switched the solvent to toluene (300 mL) to give 4-hydroxytetrahydro-2H-pyran-4-carbonitrile. Pyridine (48.5 mL, 599 mmol) was added at 65° C., followed by a slow addition of POCl₃ (27.9 mL, 300 mmol). The RM was stirred at 65° C. for 1 hour, then it was cooled to RT and water was added. The layers were separated, and the aqueous layer was extracted twice with toluene. The combined organic layers were washed with brine, dried over Na₂SO₄ and concentrated under reduced pressure to give 3,6-dihydro-2H-pyran-4-carbonitrile. The residue was mixed with toluene (300 mL) and DIBAL-H (46.9 g, 330 mmol) was added at −10° C. The reaction was stirred at −10° C. for 1 hour, then 4M HCl was added. The two layers were separated, and the aqueous layer was extracted twice with DCM. The combined organic layers were washed with brine and dried over Na₂SO₄. The organic solution was then concentrated under reduced pressure to give the title compound as solution in toluene (not stable when concentrated) (˜14% weight, not stable when concentrated). Overall yield: 63%.

¹H NMR (400 MHz, DMSO-d₆) δ 9.46 (s, 1H), 7.05 (m, 1H), 4.33 (m, 2H), 3.69 (m, 2H), 2.16 (m, 2H).

### Intermediate AB: 3,6-dihydro-2H-pyran-4-carbaldehyde

### Step 1: methyl tetrahydro-2H-pyran-4-carboxylate

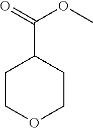

According to Ref. WO2013/66729, 2013, A1

Into a solution of oxane-4-carboxylic acid (50.0 mg, 380 μmol) in MeOH (10 mL) was added thionyl chloride (46.0 mg, 390 μmol) dropwise with stirring at RT. The resulting solution was stirred at RT for 3 hours. The RM was concentrated under reduced pressure to give the title compound, which was used directly for next step without further purification (brown oil).

¹H NMR (400 MHz, DMSO-d₆) δ 3.81 (m, 2H), 3.62 (s, 3H), 3.34 (m, 2H), 2.59 (m, 1H), 1.74 (m, 2H), 1.56 (m, 2H).

### Step 2: methyl 4-iodotetrahydro-2H-pyran-4-carboxylate

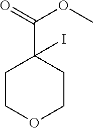

To a solution of DIPEA (528 mL, 3.03 mol) in THF (3.5 L) was added dropwise n-BuLi 2M in hexane (1.52 L, 3.03 mmol) at −10° C.˜−5° C. The RM was stirred at −10° C.˜−5° C. for 30 minutes. Methyl tetrahydro-2H-pyran-4-carboxylate (370 g, 2.43 mol) in THF (700 mL) was added dropwise into the RM at −70° C. The RM was stirred for at −70° C. for 1 hour. Iodine (1.23 g, 4.85 mol) in THF (2.4 L) was added dropwise into the RM at −70° C. The RM was stirred for at −70° C. for 1 hour. The mixture was added dropwise into 1N HCl (1.5 L) and TBME (1.5 L) and it was stirred for 30 minutes. The organic was washed with aq 30% Na₂O₃S₂ (1.8 L) and brine (700 mL). The organic layer was concentrated under reduced pressure to give the title compound (500 g, 87% pure, yield: 66%).

### Step 3: methyl 3,6-dihydro-2H-pyran-4-carboxylate

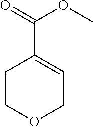

To a solution of methyl 4-iodotetrahydro-2H-pyran-4-carboxylate (500 g, 1.61 mol) in TBME (10 L) was added dropwise DBU (486 mL, 3.22 mol) at RT. The RM was stirred for at RT 2 hours, then it was filtered and to the filtrate was added HCl 9% (1.7 L). The mixture was stirred for 30 minutes. The pH of the organic was adjusted to 7 by washing with brine (2×1.5 L). The combined aqueous phase was extracted with TBME (1.5 L). After concentration, the residue was purified by distillation collects 55° C. of distillate to give the title compound (195 g, 82% pure, yield: 70%).

¹H NMR (400 MHz, DMSO-d₆) δ 6.92 (m, 1H), 4.21 (m, 2H), 3.69 (m, 5H), 2.25 (m, 2H).

### Step 4: (3,6-dihydro-2H-pyran-4-yl)methanol

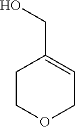

Methyl 3,6-dihydro-2H-pyran-4-carboxylate (50.0 g, 288 mmol) was mixed in THF (900 mL) under nitrogen. The solution was cooled to −10° C., followed by addition of DIBAL-H 1M in hexane (721 mL, 721 mmol) dropwise. The RM was stirred at 0° C. for 2 hours, then it was quenched by addition of 3N Rochelle's salt solution (700 mL) and MeOH (117 mL). The biphasic solution was stirred for 16 hours, and the organic phase was isolated. The aqueous phase was extracted with DCM:MeOH 10:1 (5×200 mL) and the combined organic phases were concentrated under reduced pressure to give the title compound as a liquid (36.0 g, 83% pure, yield: 90%).

¹H NMR (400 MHz, DMSO-d₆) δ 5.61 (m, 1H), 4.74 (m, 1H), 4.02 (m, 2H), 3.82 (m, 2H), 3.67 (m, 2H), 1.97 (m, 2H).

### Step 5: 3,6-dihydro-2H-pyran-4-carbaldehyde

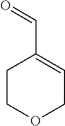

To a solution of DMP (122 g, 288 mol) in IPAc (600 mL) under nitrogen was added dropwise (3,6-dihydro-2H-pyran-4-yl)methanol (36.0 g, 262 mmol) at 0° C. The RM was stirred at RT for 3 minutes. The suspension was filtered and washed with IPAc (36 mL). The pH of the filtrate was adjusted to 8 by washing with 10% Na₂CO₃ (500 mL). The aqueous phase was extracted with IPAc (2×150 mL). The combined organic phase was concentrated under reduced pressure to 1/2 volume to give the title compound as a solution in IPAc (20.4 g, 96% pure, yield: 67%).

¹H NMR (400 MHz, DMSO-d₆) δ 9.46 (s, 1H), 7.05 (m, 1H), 4.33 (m, 2H), 3.69 (m, 2H), 2.16 (m, 2H).

### Intermediate AC: tert-butyl 4-(1-methoxy-1,3-dioxopentan-2-yl)piperazine-1-carboxylate

### Step 1: methyl 2-chloro-3-oxopentanoate

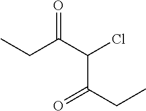

To a solution of methyl 3-oxopentanoate (10.4 kg, 80.0 mol) in DCM (67 L) was added SO₂Cl₂ (14.0 kg, 104 mol) at RT over 2.5 hours. The reaction was allowed to warm to RT and stirred for 16 hours. The RM was concentrated under reduced pressure and the residue was dissolved in DCM (20 L) and washed with water (10 L), brine (10 L), dried over Na₂SO₄ and filtered. The filtrate was concentrated under reduced pressure to give the title compound as a light yellow liquid. (14.4 kg, 73% pure, yield: 80%).

¹H NMR (400 MHz, CDCl₃-d) δ 4.65 (s, 1H), 3.68 (s, 3H), 2.59 (m, 2H), 0.96 (t, 3H)

### Step 2: tert-butyl 4-(1-methoxy-1,3-dioxopentan-2-yl)piperazine-1-carboxylate

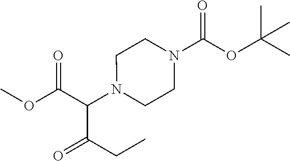 [SMILES: CCC(=O)C(C(=O)OC)N1CCN(C(=O)OC(C)(C)C)CC1]

To a solution of methyl 2-chloro-3-oxopentanoate (12.1 kg, 53.7 mol) in dry ACN (53 L) was added Et₃N (22.3 L, 161 mol) over 1.5 hours, followed by dropwise addition of tert-butyl piperazine-1-carboxylate (10.0 kg, 53.7 mol) in ACN (50 L) over 2.5 hours. The reaction was stirred at 60° C. for 16 hours. The RM was filtered and washed with EtOAc (10 L). The filtrate was then concentrated under reduced pressure and the residue was dissolved in EtOAc (45 L) and washed with water (45 L), dried over Na₂SO₄ and filtered. The filtrate was concentrated under reduced pressure and the residue was purified by column chromatography (Silica column 150 mm×800 mm×70 mm, eluent heptane:EtOAc 100:0 to 90:10) to give the title compound. (16.1 kg, 93% pure, yield: 89%).

HPLC: Rt=3.681/5.513 min; HPLC 1

### Intermediate AD: ethyl 2-bromo-3-oxopentanoate

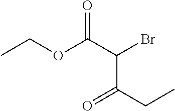

Ethyl 3-oxopentanoate (10.0 g, 65.9 mmol) was dissolved in DCM (100 mL), NBS (12.3 g, 69.2 mmol) was added, followed by TsOH·H₂O (2.53 g, 13.2 mmol). The RM was stirred at RT for 2.5 hours. The RM was filtered through Hyflo and washed with DCM. The filtrate was extracted with water (3×50 mL). The organic layer was washed with DCM (3×200 mL). The combined organic layers were dried through a phase separator and concentrated under reduced pressure to give the title compound as a colourless liquid (15.9 g, 90% pure, yield: 97%).

LC-MS: Rt=0.90 min; MS m/z [M−H]⁻ 221.1/223.1; UPLC-MS 5

### Intermediate AE: tert-butyl (2R)-4-(1-ethoxy-1,3-dioxopentan-2-yl)-2-methylpiperazine-1-carboxylate

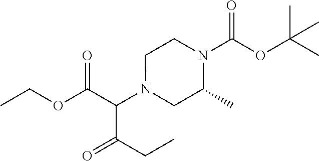 [SMILES: CCOC(=O)C(C(=O)CC)N1CCN(C(=O)OC(C)(C)C)[C@H](C)C1]

Ethyl 2-bromo-3-oxopentanoate (Intermediate AD) (15.6 g, 62.9 mmol) and tert-butyl (R)-2-methylpiperazine-1-carboxylate (13.2 g, 66.1 mmol) were mixed in ACN (250 mL) and K₂CO₃ (17.4 g, 126 mmol) was added. The reaction was stirred at RT for 4.5 hours. The suspension was filtered and the cake was washed with ACN. The filtrate was concentrated under reduced pressure. The yellow oil was extracted with DCM (4×200 mL) and water (2×150 mL). The organic layer was dried through a phase separator and concentrated under reduced pressure. The residue was adsorbed onto Isolute and purified by column chromatography (RediSep Column: Silica 330 g, eluent heptane:EtOAc 100:0 to 75:25) The product containing fractions were combined and concentrated under reduced pressure to get the title compound as a colourless oil (17.3 g, 99% pure, yield: 79%).

LC-MS: Rt=1.20/1.42 min; MS m/z [M+H]⁺ 343.3, m/z [M−H]⁻ 341.1; UPLC-MS 5

### Intermediate AF: diethyl 2-(4-(tert-butoxycarbonyl)piperazin-1-yl)-3-oxoheptanedioate

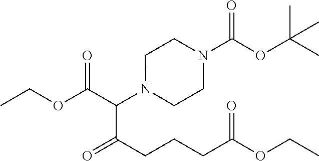 [SMILES: CCOC(=O)CCCC(=O)C(C(=O)OCC)N1CCN(C(=O)OC(C)(C)C)CC1]

1M Lithium bis(trimethylsilyl)amide in THF (72.0 mL, 72.0 mmol) was purged with argon at −78° C. A solution of tert-butyl 4-(2-ethoxy-2-oxoethyl)piperazine-1-carboxylate (10.0 g, 36.0 mmol) in THF (90 mL) was added dropwise within 15 minutes. The RM was stirred at −40° C. for 1.5 hours. The RM was cooled to −78° C. and ethyl 5-chloro-5-oxopentanoate (7.07 g, 39.6 mmol) was added dropwise within 20 minutes. The RM was allowed to warm to RT and it was stirred at RT for 5 hours, then it was quenched with water (150 mL). The RM was extracted with EtOAc (2×500 mL). The combined organic layers were washed with brine (2×75 mL) and dried trough a phase separator. The filtrate was concentrated under reduced pressure to give an oil (16.6 g). The crude product was adsorbed onto Isolute and purified by column chromatography (RediSep Column: Silica 120 g, eluent heptane:EtOAc 95:5 to 85:15). The product containing fractions were combined and concentrated under reduced pressure to give the title compound as a slightly yellow oil (8.29 g, 99% pure, yield: 55%).

LC-MS: Rt=1.07/1.23 min; MS m/z [M+H]⁺ 415.4, m/z [M−H]⁻ 413.3; UPLC-MS 8

### Intermediate AG: 7-(tert-butyl) 1-methyl (R)-4-methyl-3-oxoheptanedioate

### Step 1: tert-butyl (R)-5-((S)-4-benzyl-2-oxooxazolidin-3-yl)-4-methyl-5-oxopentanoate

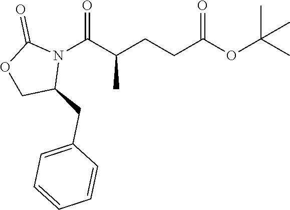 [SMILES: C[C@H](CCC(=O)OC(C)(C)C)C(=O)N1C(=O)OC[C@@H]1Cc1ccccc1]

Ti(OiPr)₄ (8.82 mL, 29.8 mmol) and TiCl₄ in DCM (92.0 mL, 92.0 mmol) were added to dry DCM (500 mL) at 0° C. under nitrogen. After 10 minutes, DIPEA (21.0 mL, 120 mmol) was added. The resulting brown solution was stirred for 10 minutes and (S)-4-benzyl-3-propionyloxazolidin-2-one (26.7 g, 115 mmol) was added as a solid. The RM was stirred at 0° C. for 1.5 hours, then tert-butyl acrylate (24.9 mL, 172 mmol) was added dropwise. The RM was stirred at RT for 16 hours. The RM was quenched with aq sat NH₄Cl and extracted with DCM. The organic layer was dried over Na₂SO₄ and concentrated under reduced pressure. The crude product was purified by column chromatography (RediSep Column: Silica 330 g, eluent heptane:EtOAc 100:0 to 35:65). The product containing fractions were combined, concentrated under reduced pressure and dried under HV to give the title compound as a yellow oil (30.0 g, 100% pure, yield: 73%).

LC-MS: Rt=1.23 min; no mass observed; UPLC-MS 1

### Step 2: (R)-5-(tert-butoxy)-2-methyl-5-oxopentanoic acid

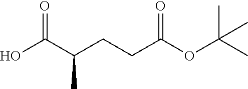

To a stirred solution of tert-butyl (R)-5-((S)-4-benzyl-2-oxooxazolidin-3-yl)-4-methyl-5-oxopentanoate (30.0 g, 83.0 mmol) in THF (300 mL) and water (100 mL) was added at 0° C. hydrogen peroxide 30% in water (35.6 mL, 581 mmol), followed by a solution of LiOH·H2O (6.97 g, 166 mmol) in water (50 mL) and the RM was stirred for 1 hour. The RM was quenched at 0° C. with 10% Na₂SO₃ solution. THF was evaporated. To the resulting aqueous residue was added 2N NaOH (6 mL) and the aqueous mixture was extracted twice with DCM. The aqueous layer was cooled to 0° C. and acidified to pH 1-2 with 2N HCl, then extracted twice with EtOAc and the combined organic layers were dried over Na₂SO₄ and concentrated under reduced pressure to give the title compound as a colourless oil.

(16.3 g, 100% pure, yield: 97%).

### Step 3: 7-(tert-butyl) 1-methyl (R)-4-methyl-3-oxoheptanedioate

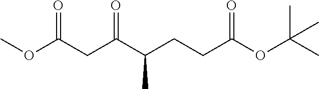 [SMILES: COC(=O)CC(=O)[C@H](C)CCC(=O)OC(C)(C)C]

To a stirred solution of (R)-5-(tert-butoxy)-2-methyl-5-oxopentanoic acid (16.3 g, 80.0 mmol) in THF (150 mL) was added CDI (19.6 g, 121 mmol) at RT and the RM was stirred at RT for 16 hours. A solution of methyl potassium malonate (25.1 g, 161 mmol), powdered magnesium chloride (15.3 g, 161 mmol), DMAP (983 mg, 8.04 mmol) and Et₃N (44.9 mL, 322 mmol) in THF (150 mL) was stirred at RT for 2 hours, then the first solution was added and the RM was stirred at 80° C. for 22 hours. The RM was diluted with EtOAc and water, extracted twice with EtOAc and the combined organic layers were washed with water and brine, dried over Na₂SO₄ and concentrated under reduced pressure. The crude product was purified by column chromatography (RediSep Column: Silica 220 g, eluent heptane:EtOAc 100:0 to 60:40). The product containing fractions were combined, concentrated under reduced pressure and dried under HV to give the title compound as a yellow oil (16.0 g, 100% pure, yield: 77%).

LC-MS: Rt=0.93 min; no mass observed; UPLC-MS 1

### Intermediate AH: tert-butyl (1S,6S)-2,5-diazabicyclo[4.2.0]octane-2-carboxylate

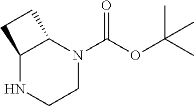

Tert-butyl 2,5-diazabicyclo[4.2.0]octane-2-carboxylate (2.20 g, 10.0 mmol) was purified by preparative chiral SFC (instrument: SEPIATEC PREP SFC 100; column: Chiralpak IG 250 mm×30 mm 5 μm; eluent: A: 13% MeOH+0.05% NH₃, B: 87% scCO₂; flow rate: 80.0 mL/min; detection: UV; injection volume: 1.2 mL; gradient: isocratic A: 13%, B: 87%; oven temperature: 40° C.; BPR: 130 bar).

Peak 1: tert-butyl (1S,6S)-2,5-diazabicyclo[4.2.0]octane-2-carboxylate (Intermediate AH)

Colourless crystals (927 mg, 99% pure, yield: 42%)

Chiral HPLC (C-HPLC 14): Rt=1.82 min, 99.5% ee

Peak 2: tert-butyl (1R,6R)-2,5-diazabicyclo[4.2.0]octane-2-carboxylate

Colourless crystals (913 mg, 99% pure, yield: 42%)

Chiral HPLC (C-HPLC 14): Rt=3.31 min, 99.5% ee

The stereochemistry of intermediate AH was determined by single-crystal X-ray.

### Intermediate AI: 3-bromo-1H-1,2,4-triazol-5-amine

### Step 1: 3,5-dibromo-1-(methoxymethyl)-1H-1,2,4-triazole

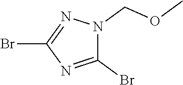

To a solution of NaH (846 g, 21.2 mol, 60%) in DMF (12 L) was added 3,5-dibromo-1H-1,2,4-triazole (4.00 kg, 17.6 mol) at 10° C. The resulting solution was stirred at 10° C. for 1 hour. This was followed by the addition of chloro(methoxy)methane (1.70 kg, 21.2 mol) dropwise at 20° C. The mixture was stirred at RT overnight. The reaction was quenched with H₂O (20 L). The resulting solution was extracted with TBME (2×7 L). The combined organic layers were washed with 10% NaCl (2×7 L), dried over Na₂SO₄ and concentrated under reduced pressure at 40° C. The residue was triturated with heptane (3 L) to give the title compound as a white solid. (4.20 kg, 94% pure, yield: 83%).

HPLC: Rt=3.042 min; HPLC 1

### Step 2: 3-bromo-1-(methoxymethyl)-1H-1,2,4-triazol-5-amine

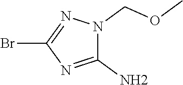

3,5-Dibromo-1-(methoxymethyl)-1H-1,2,4-triazole (800 g, 2.78 mol) was dissolved in 25% NH₃H₂O (2.89 L, 16.3 mol) and MeOH (80 mL) at RT. The mixture was stirred at 120° C. for 18 hours. The mixture was cooled to 5-10° C., the solids were collected by filtration and washed with water (200 mL). The cake was dried under vacuum at 60° C. to give the title compound as a white solid (440 g, 91% pure, yield: 70%).

HPLC: Rt=1.701 min; HPLC 1

### Step 3: 3-bromo-1H-1,2,4-triazol-5-amine

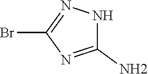

To a solution of 3-bromo-1-(methoxymethyl)-1H-1,2,4-triazol-5-amine (329 g, 1.45 mol) in MeOH (1.5 L) was added HBr (4.39 kg, 21.7 mol) at RT. The mixture was stirred at 100° C. for 18 hours. The mixture was adjusted to pH=7.0-7.5 with 10% NaOH at 20-30° C. and extracted with EtOAc (10×3 L). The combined organic phase was dried over Na₂SO₄ and concentrated under reduced pressure at 50° C. to give the title compound as a white solid. (177 g, 95% pure, yield: 71.5%).

HPLC: Rt=0.702 min; HPLC 1

HPLC: Rt=1.902 min; HPLC 2

### Intermediate AI: 3-bromo-1H-1,2,4-triazol-5-amine

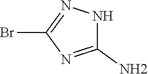

To a solution of 1H-1,2,4-triazole-3,5-diamine (300 g, 3.03 mol) in HBr/H₂O (2.4 L) was added dropwise NaNO₂ (313 g, 4.54 mol) in water (782 mL) at 0° C. over 1.5 hours. The reaction was allowed to warm to RT and stirred for 1 hour. The reaction was stirred at 100° C. and for 16 hours. The RM was cooled to RT, filtered and the pH of the mixture (66 batches combined) was adjusted to 4 by addition of 10% NaOH. The mixture was extracted with EtOAc (2×55 L), dried over Na₂SO₄ and filtered. The organic phase was concentrated under reduced pressure to give the title compound (3.40 kg, 49% pure, yield: 5%). The pH of the aqueous phase was adjusted to 7-7.5 by 10% NaOH. Then it was extracted with EtOAc (10×35 L), dried over Na₂SO₄ and filtered. The organic phase was concentrated under reduced pressure to give the title compound (6.00 kg, 77% pure, yield: 14%). Total yield: 19%.

HPLC: Rt=1.933 min; HPLC 2

### Intermediate AJ

A synthetic route to provide intermediate AJ is provided below:

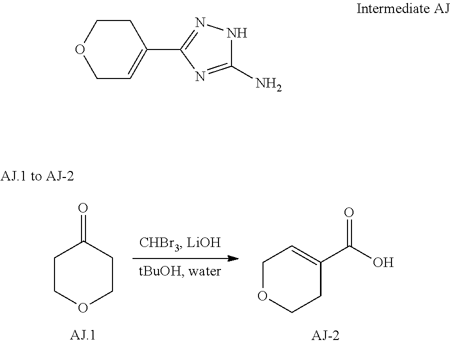

Water was charged and stirred at 25° C., then LiOH H₂O added and the mixture cooled to 25° C. t-BuOH and benzyl triethylammonium chloride were subsequently added, then the mixture heated to 45° C. AJ-1 and CHBr₃ were stirred together at 25° C. for 10 mins, then this mixture added dropwise into the first aqueous mixture at a temperature s 55° C. The resulting mixture was cooled to 45° C. and stirred 45° C. at for 20 hours. The reaction mixture was filtered, and the mother liquid concentrated. The residue was cooled to 0° C. and conc. HCl added dropwise. The resulting mixture was extracted using ethyl acetate (EA), then Na₂SO₄ added and stirred for 30 min. The solid was filtered and the mother liquid concentrated under reduced pressure. A brown solid was obtained, and EA added to the solid. The mixture was heated to 65° C.-58° C. and stirred for 2 hours minimum, then heptane added dropwise then the mixture cooled slowly while stirring. The product was then filtered and dried under vacuum, 41.57% yield.

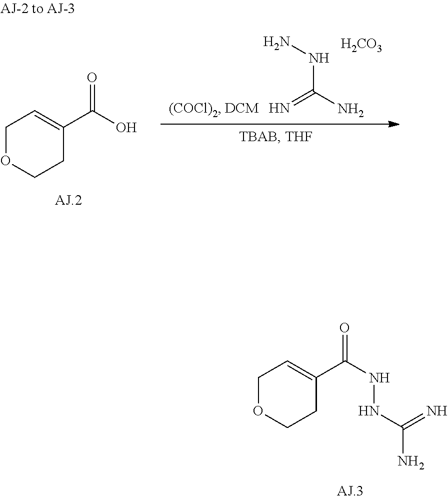

### TBAB Means Tetrabutyl Ammonium Bromide

To a flask was charged intermediate AJ.2, DMF and DCM. Oxalyl chloride was added dropwise then the reaction mixture stirred at room temperature for 2 hours. The mixture was concentrated under vacuum and the acid chloride used without further purification.

Hydrazinecarboximidamide carbonate and tetrabutyl ammonium bromide were charged to a flask. 1,4-Dioxane was then added and the mixture was stirred under N₂ at 0° C. A solution of the freshly prepared acyl chloride as described above in 1,4-dioxane was added dropwise over 30 min. The reaction mixture was warmed to room temperature and stirred overnight. The mixture was filtered, the wet cake was washed with THF. The product was obtained as a white solid, 88.2% yield.

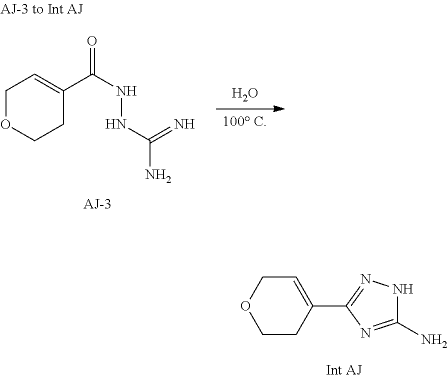

Intermediate AJ-3 and water were charged to a flask. The suspension was heated to 100° C., then the clear solution was stirred at 100° C. for 18 hours. NaCl was added to the mixture which was then was cooled slowly to 20° C. After filtration and drying, a white solid of Int AJ was obtained, 75.38% yield.

An alternative synthesis of intermediate AJ is provided below:

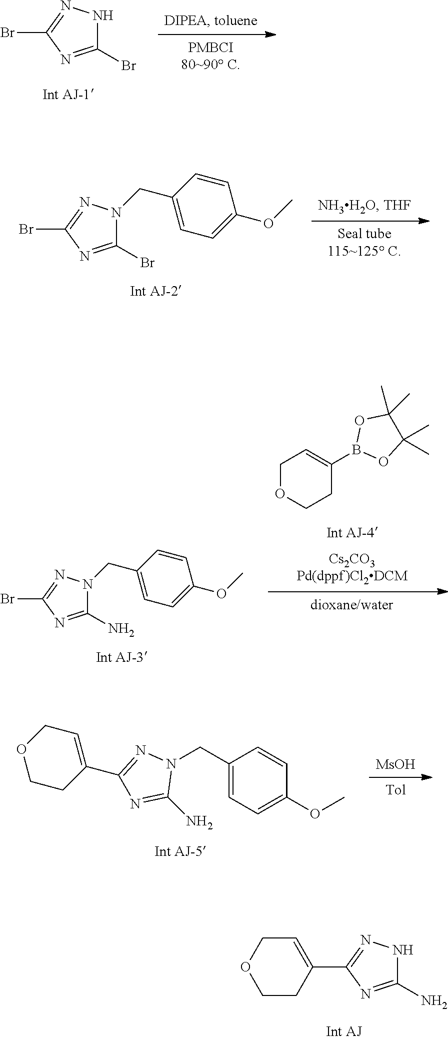

### Step AJ-1′ to AJ-2′

To a flask of toluene was charged compound AJ-1′, DIPEA and PMBCl (para-methoxybenzyl chloride). The mixture was heated at 80-90° C. for 2.5 hours then was cooled to 10-20° C. Ethyl acetate was charged followed by water. The organic layer was separated and was washed with 7% Na₂SO₄ aqueous solution then the organic layer concentrated, then treated with MeOH. The mixture was heated to 40° C. for 30 minutes. Water was added, then the mixture was cooled down to 10-20° C. The resulted suspension was filtered to obtain compound AJ-2′ as a solid (87% yield).

### Step AJ-2′ to AJ-3′

To a sealable tube was charged THF, NH₃·H₂O and compound AJ-2′. The tube was tightly sealed and heated at 120° C. for 20 hours. The mixture was then cooled, concentrated under vacuum, then the resulting suspension filtered. The product was redissolved in DCM, dried with Na₂SO₄ then filtered. The resulted solution was concentrated and Int AJ-3′ was obtained by recrystallisation in heptane as a solid (83.4% yield).

### Step AJ-3′ to AJ-5′

To a flask under nitrogen atmosphere was charged dioxane, water, compound AJ-3′, compound AJ-4′, Cs₂CO₃ and Pd(dppf)Cl₂·DCM. The mixture was heated at 80-90° C. for 2 hours then cooled to ambient temperature. The suspension was stirred for an additional 1 hour then filtered. Compound AJ-5′ was obtained by filtration as a solid (64.4% yield).

### Step AJ-5′ to AJ-6′

To a flask was charged toluene, compound AJ-5′, and MsOH. The mixture was heated at 65-75° C. for 3 hours then cooled. The upper layer was removed and the bottom layer was treated with water and Na₂CO₃ to pH 8-9. The aqueous phase was then extracted with DCM and MeTHF. The combined organic layer was dried by Na₂SO₄ then filtered. The solution was concentrated to obtain crude compound AJ-6′, then was purified by slurry in heptane for 30 minutes to obtain compound AJ-6′ as a solid (75.6% yield).

### Alternative Route to Intermediate Q Step 2 Product:

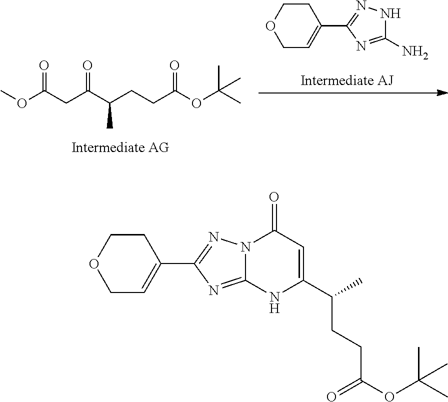

To a sealed tube Int AJ (1.0 eq.) and Int AG (1.6 eq.) were added. t-Butanol (2.7V), and TEOS (tetraethyl orthosilicate, 2.0 eq.) were subsequently added. To the stirring mixture trifluoroacetic acid (1.0 eq.) was slowly added. The formed suspension was stirred at 85° C. for 18 h and Int AJ (1.6 eq.) was added. The mixture was further heated at 85° C. for another 64 h, which was cooled to room temperature and concentrated under vacuum. The obtained oil was purified with column chromatography (MeOH/DCM=0-5%). A light yellow solid was obtained.

This alternative route has significant advantages of avoiding unstable intermediate compounds and reducing the number of process steps, Surprisingly, the adoption of TEOS as reagent has successfully enabled a good yield alongside good enantiomeric excess.

Separation of enantiomers may take place at various stages of the synthetic process. Protecting groups may also be used at different stages of the process according to procedures well known to synthetic chemist.

The following intermediates may also be used in the processes to make compounds of the invention:

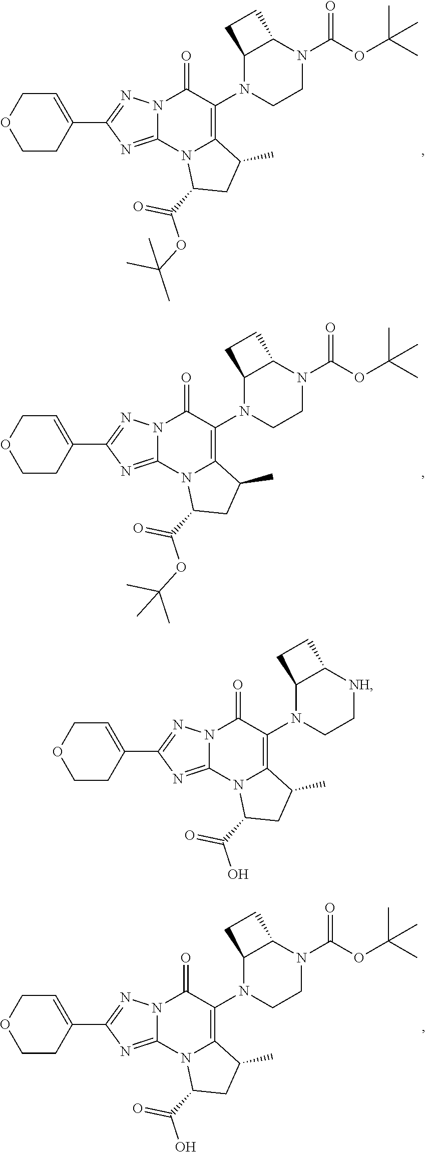

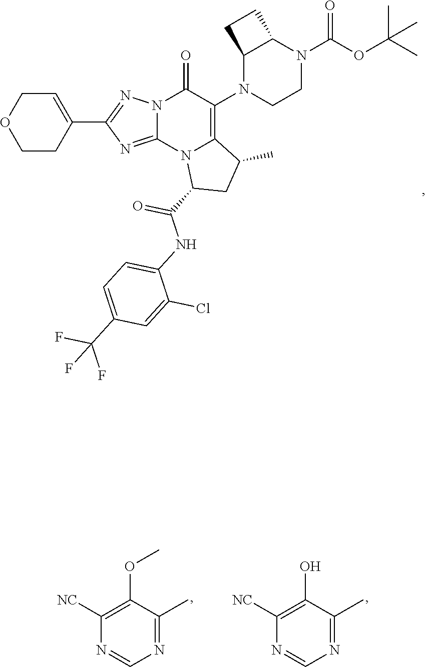

### Intermediate AK

Intermediate AK, which is similar to intermediate U, can be made according to the following procedure:

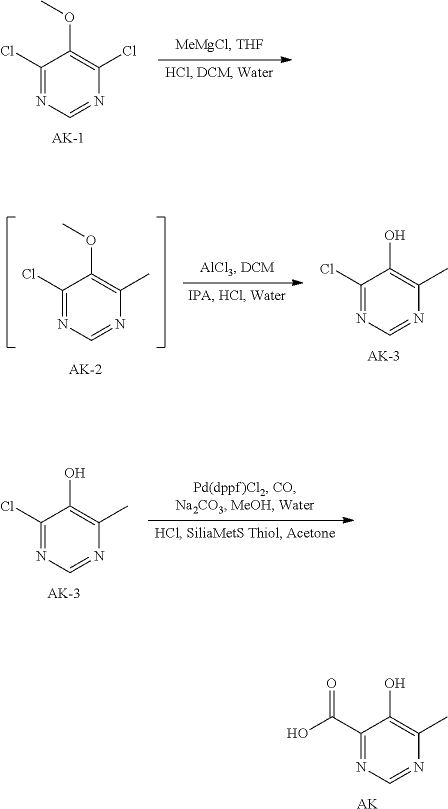

### Step AK-1 to AK-2

4,6-Dichloro-5-methoxypyrimidine (50.0 g, 279 mmol) was dissolved in THF (400 mL) under nitrogen and cooled to 5° C. Methylmagnesium chloride 3M (102 mL, 307 mmol) was added dropwise. The RM was stirred at 5° C. for 1 hour, then it was quenched by addition of HCl 1N (250 mL) and extracted twice with TBME (2×250 mL). The combined organic layers were washed with brine (250 mL) and concentrated under reduced pressure. The residue was treated with MeOH to give the AK-2 in solution.

¹H NMR (400 MHz, DMSO-d₆) δ 8.66 (s, 1H), 3.86 (s, 3H), 2.50 (s, 3H)

### Step AK-2 to AK-3

To a flask was charged with AK-2 (10.0 g by AK-2 content) solution in THF, solvent swap to DCM (100 mL) under reduced pressure. The mixture was then cooled down to 0° C. followed by charging AlCl3 (11.2 g, 1.5 eq.) and the mixture was stirred at 35-45° C. for 17 hours. After reaction completion, the mixture was cooled to 0° C. and quenched with HCl (1N in water). MeOH (50 mL) was charged afterwards, and the mixture was extracted by DCM/MeOH (1/9, 130 mL) four times. The combined organic layer was dried over Na₂SO₄ and concentrated under reduced pressure. Product AK-3 was isolated by column chromatography as a solid (8.4 g).

### Step AK-3 to AK

AK-3 (20 g, 1.0 eq) was charged in a vial, added 0.5 mol % Pd(dppf)Cl₂, Na₂CO₃ (1.1 eq.) and H₂O (4.0 eq.) and MeOH (10 V). The mixture was heated to 100° C. under 12 bar CO atmosphere for 30 hours. The solution was treated with MeOH and Pd scavenger, then was filtered. The clear solution was concentrated and treated with water, followed by addition of HCl (2.0 eq.). The suspension was filtered and AK was obtained as a white solid (84% yield).

### Synthetic Schemes for the Compounds of Formulae 1b, 1c, 1d, 1e and 1f

Processes are provided to make to compounds of formulae 1b, 1c, 1d, 1e and 1f. Unless otherwise stated, the groups of the process schemes are as defined in the embodiments and preferences herein. The syntheses can be modified to make variants under formula (I), according to procedures known to the skilled chemist.

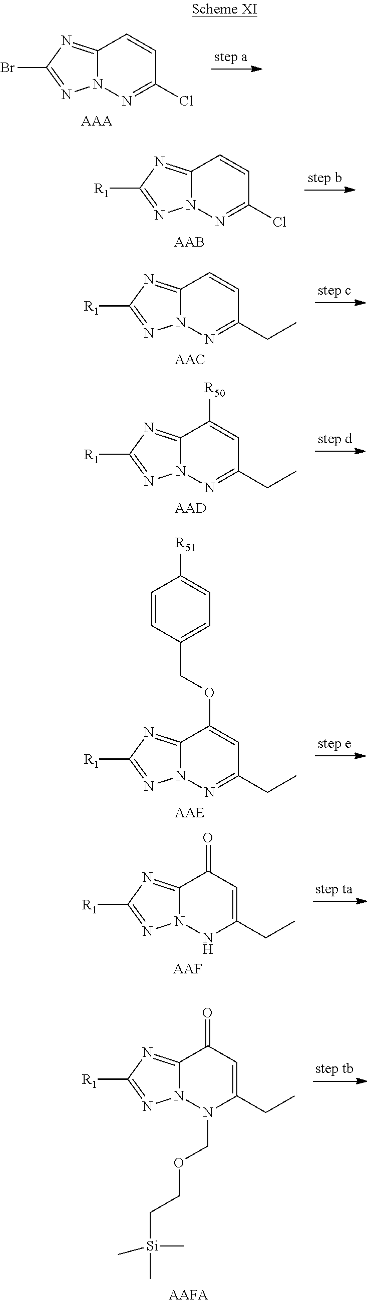

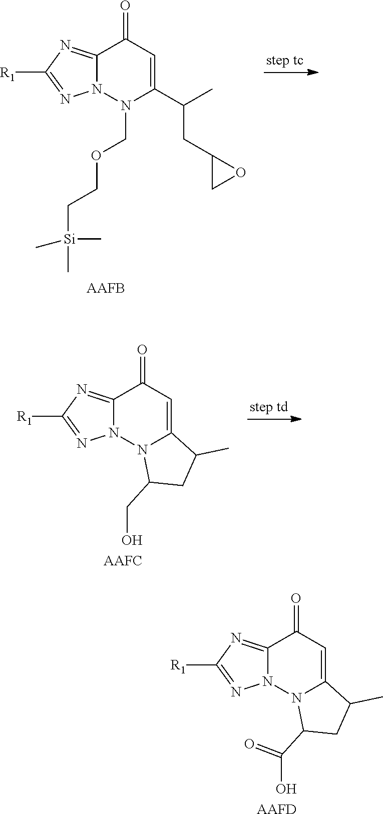

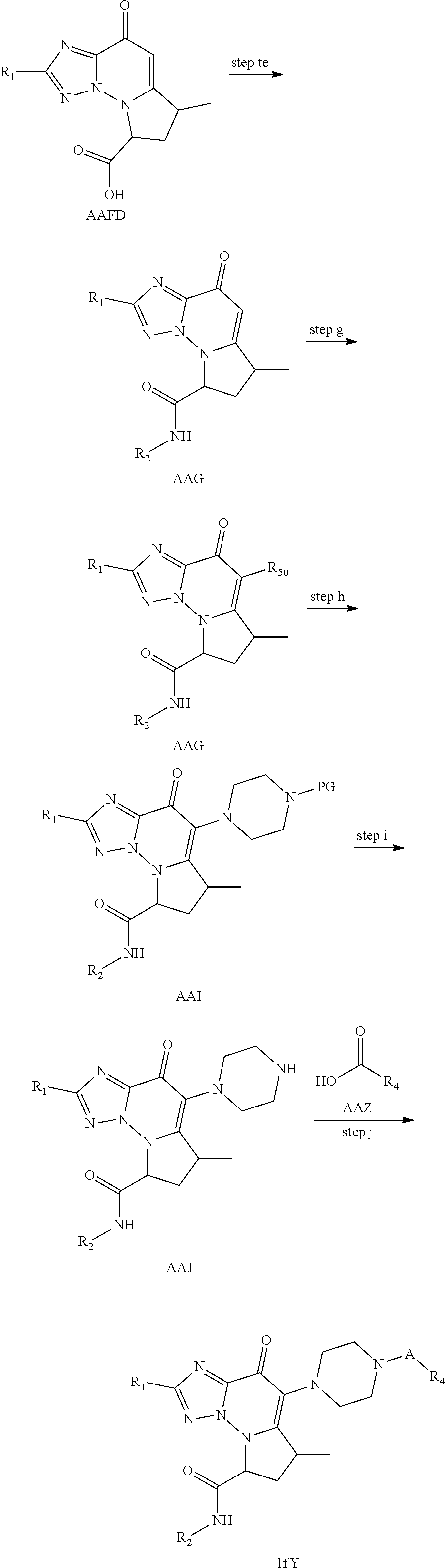

A process for preparing a compound of formula 1fY (Scheme XI) comprising steps a, b, c, d, e, ta, tb, tc, td, te, g, h, i, and j. It is understood that the order of process steps a, b, c, d, e, ta, tb, tc, td, te, g, h, i, and j may change to optimize the synthesis as necessary. The compound of formula 1fY can be obtained via coupling reaction step j by reacting compound of formula AAJ with compound AAZ wherein R₄ is defined above. The coupling reaction can be an amide formation. The coupling reaction step can be carried out with for example HATU ((1-[Bis(dimethylamino)methylene]-1H-1,2,3-triazolo[4,5-b]pyridinium 3-oxide hexafluorophosphate) or alternatively Ghosez reagent (1-Chloro-N,N,2-trimethylpropenylamine) preferably in a one or two step procedure. Alternatively, further alternative amide coupling methods are known in the art. For examples of amide bond formations, see Mantalbetti, C. A. G. N and Falque, V., Amide bond formation and peptide coupling, Tetrahedron, 2005, 61(46), pp 10827-10852 and references cited therein. Compound of formula AAJ can be prepared comprising step i of deprotecting PG from the compound of formula AAI, wherein PG represents a suitable protecting group, preferably a BOC group and the other substituents are as defined above. There are many known ways of deprotecting BOC groups. The deprotection step can be carried out with for example TFA or HCl in a solvent for example dichloromethane or dioxane.

Compound of formula AAI can be prepared comprising step h starting from a compound of formula AAH wherein R₅₀ represents halo, particularly bromo and wherein PG represents a suitable protecting group for example a BOC group and the other substituents are as defined above. Step h can be a nucleophilic aromatic substitution reaction and can be carried out by combining compound of formula AAH with an amine for example tert-butyl piperazine-1-carboxylate or alternatively piperazine. A stoichiometric excess of the amine can be used, preferably between 2 and 50 mole equivalents in an organic solvent for example DMSO or NMP. The reaction is preferably stirred at a temperature of approximately 80-140° C. and can be carried out in a capped tube. An alternative method for step h can use Buchwald-Hartwig conditions using a amine for example tert-butyl piperazine-1-carboxylate, a ligand such as Brettphos or RuPhos or RuPhos hybrid with a palladium catalyst such as RuPhos Pd G1, RuPhos Pd G4 or [PdCl(allyl)]2 in the presence of a base such as K₂CO₃ or Cs₂CO₃ or tert-BuONa in an organic solvent such as dioxane or THF. The reaction is preferably stirred at a temperature of approximately 80-120° C., and is preferably carried out under an inert gas such as nitrogen or argon. Alternatively, further alternative Buchwald-Hartwig coupling methods are known in the art, for examples of methods, see B. T. Ingoglia et al Biaryl monophosphine ligands in palladium-catalyzed C—N coupling: An updated User's guide, Tetrahedron, 2019, 75(32), pp 4199-4211 and references cited therein.

Compound of formula AAH can be prepared comprising step g wherein compound of formula AAG wherein the substituents are defined as above is halogenated. Step g can be carried out using a halogenating reagent such as N-bromosuccinimide or N-iodosuccinimide in a solvent for example DMF or acetonitrile. The reaction is preferably stirred at a temperature of approximately 20-80° C.

Compound of formula AAG can be prepared comprising steps ta, tb, tc td and te starting from compound of formula AAF wherein the substituents are defined as above using analogous conditions for steps ta, tb, tc, td and te as those described herein.

Compound of formula AAF can be prepared comprising step e starting from compound of formula AAE wherein R₅, represents H or methoxy and the other substituents are as defined above. Many methods of cleaving benzyl or para-methoxybenzyl groups are known in the art. Step e can be carried out in the presence of an acid such as TFA or HCl or HBr, preferably in stoichiometric excess in a solvent such as dichloromethane or dioxane and is preferably stirred at a temperature of approximately 20-80° C. An alternative method for step e can use hydrogenation conditions in the presence of a hydrogen atmosphere and a catalyst such as Pd/C or palladium hydroxide/C. The reaction is preferably stirred at a temperature of approximately 20-50° C. in an organic solvent such as ethanol or methanol.

Compound of formula AAE can be prepared comprising step d starting from compound of formula AAD wherein R₅₀ represents halo, particularly bromo and the other substituents are as defined above. Step d comprises reacting 2-10 mol equivalents of an alcohol for example benzyl alcohol or para-methoxybenzyl alcohol with 2-5 mol equivalents of a base such as sodium hydride in an organic solvent such as THF or dioxane with stirring at a temperature of approximately 20-40° C., preferably 20° C. for approximately 10-60 minutes. A compound of formula AAD is then added and stirring is continued at a temperature of approximately 20-100° C., preferably 20-60° C. The reaction is preferably carried out under an inert gas such as nitrogen or argon. An example method is described in WO2021/222522, 2021, A1 page 574.

Compound of formula AAD wherein R₅₀ represents halo, particularly bromo or iodo, and the other substituents are as defined above, can be prepared comprising step c starting from compound of formula AAC. Step c comprises reacting a compound of formula AAC with a base such as LiTMP (lithium tetramethylpiperidide) or LDA (lithium diisopropylamide) with stirring in a solvent such as THF at a temperature of approximately −78° C. to 20° C. under an inert gas such as nitrogen or argon. After stirring for an appropriate time, approximately 30 minutes to 3 hours, a halogenating reagent such as bromine or iodine is then added at a temperature of approximately −78° C. to 20° C. and stirring is continued. Other suitable halogenating agents are known in the art.

Compound of formula AAC wherein R₁ and R₃ are as defined above can be prepared comprising step b starting from compound of formula AAB. Step b can be a Suzuki or Negishi or Stille or Kumada cross-coupling reaction and comprises reacting a compound of formula AAB with R₃n-MX wherein R₃ is as defined above, n is 1, 2, 3 or 4 and MX represents for example B(OH)₂, BPin (Pin represents boronic acid pinacol ester) BF₃K, B(MIDA), Sn, Zn, Mg-Halo. Example Negishi cross-coupling conditions comprise reacting compound of formula AAD with an alkyl zincate for example diethylzinc, preferably in stoichiometric excess for example 2-10 mol equivalents, in the presence of a catalyst such as PdCl₂(dppf) or Pd(PPh₃)₄ in a suitable solvent such as THF at a temperature of approximately 20-120° C., preferably 20-80° C. under an inert gas such as nitrogen or argon.

Compound of formula AAB wherein R₁ is as defined above can be prepared comprising step a from compound AAA. Step a can be a Suzuki or Negishi or Stille or Kumada cross-coupling reaction and comprises reacting a compound of formula AAA with R₁n-MX wherein R₁ is as defined above, n is 1, 2, 3 or 4 and MX represents for example B(OH)₂, BPin (Pin represents boronic acid pinacol ester) BF₃K, Sn, Zn, Mg-Halo. Example Suzuki cross-coupling conditions comprise reacting compound of formula AAA with R₁-BPin in the presence of a catalyst such as PdCl₂(dppf) or Pd(PPh₃)₄ and a base such as K₃PO₄ or potassium carbonate in a suitable solvent mixture such as DMF, THF or dioxane or water at a temperature of approximately 20-120° C., preferably 20-80° C. under an inert gas such as nitrogen or argon. An example method is described in CN112707908 A page 32.

Compound of formula AAA can be prepared according to the method described in CN112707908 A page 31.

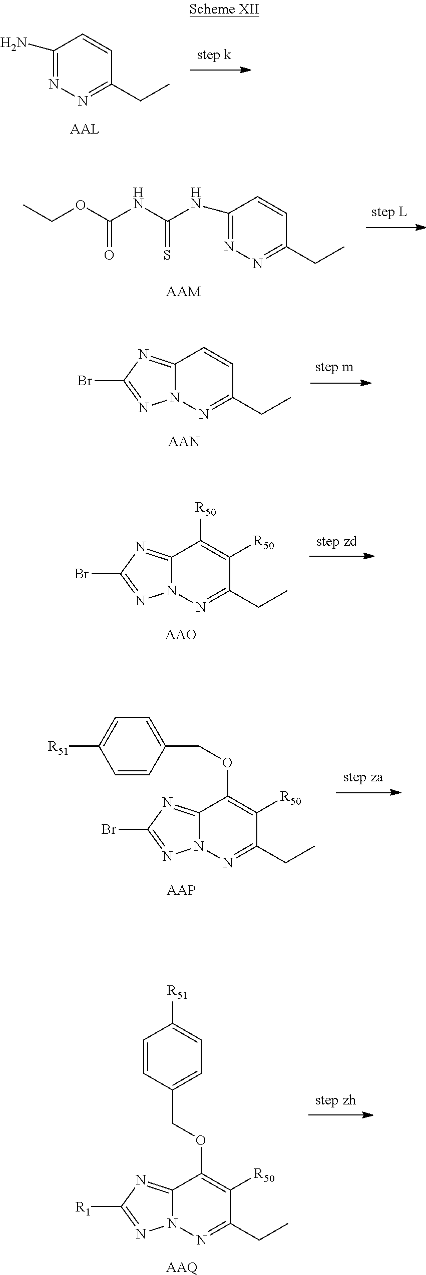

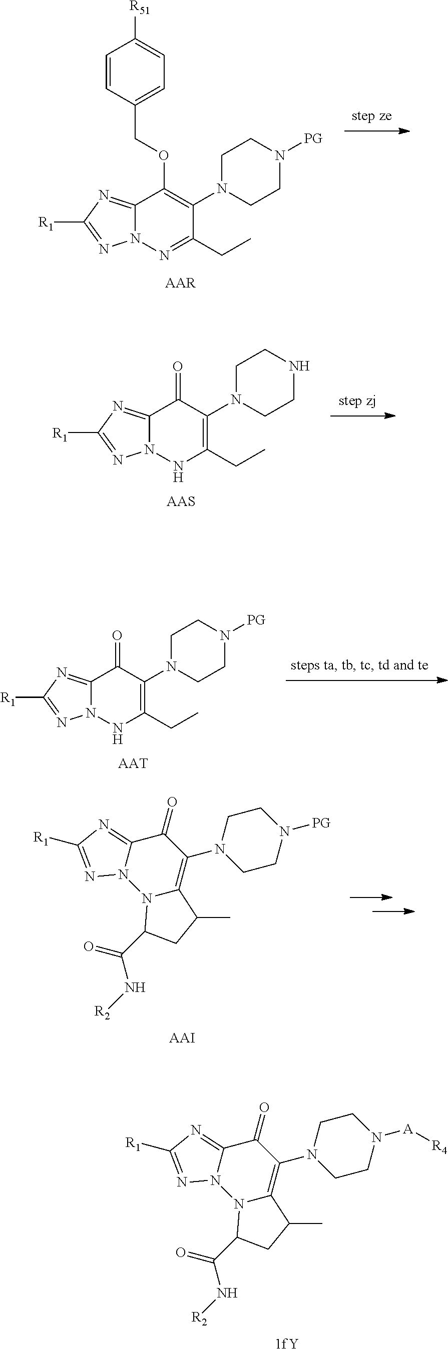

Alternatively compound of formula 1fY can be prepared according to the route shown in Scheme XII comprising steps k, L, m, zd, za, zh, ze, zj, ta, tb, tc, td and te to make the intermediate AAI which can be subsequently converted to compound 1fY using methods as described herein. Methods comprising steps zd, za, zh, ze, zj and zf to prepare compounds of formulas AAP, AAQ, AAR, AAS and AAT can be performed using analogous conditions as those described above for Scheme XI. It is understood that the order of process steps k, L, m, zd, za, zh, ze, zj, ta, tb, tc, td and te may change to optimize the synthesis as necessary.

Compound of formula AAO wherein R₅₀ represents halo, particularly bromo or chloro, and the other substituents are as defined above, can be prepared comprising step m starting from compound of formula AAN. Step m comprises reacting a compound of formula AAN with a halogenating agent such as PCl₅ or PBr₃ in stoichiometric excess for example 2-10 mol equivalents in a sealed tube at a temperature of approximately 200-270° C., preferably 250-270° C. for approximately 1-10 hours. An example method is described in J. Org. Chem., Vol. 39, No. 15, 1974, page 2146.

Compound of formula AAN wherein R₃ is as defined above, can be prepared comprising step L starting from compound of formula AAM. Step L comprises reacting a compound of formula AAM with hydroxylamine in the presence of a base such as triethylamine and a solvent such as ethanol or methanol at a temperature of approximately 60-100° C. The product of this reaction is then reacted with tert-butyl nitrite in the presence of CuBr₂ in a solvent such as acetonitrile at a temperature of approximately 20-50° C. An example method is described in CN112707908 A page 31.

Compound of formula AAM wherein R₃ is as defined above, can be prepared comprising step k starting from AAL. Step k comprises reacting a compound of formula AAL with ethoxycarbonyl isothiocyanate in a solvent such as dichloromethane at a temperature of approximately 0-20° C. for 2-18 hours.

Compound of formula AAL is commercially available, or methods for their preparation are known in the art.

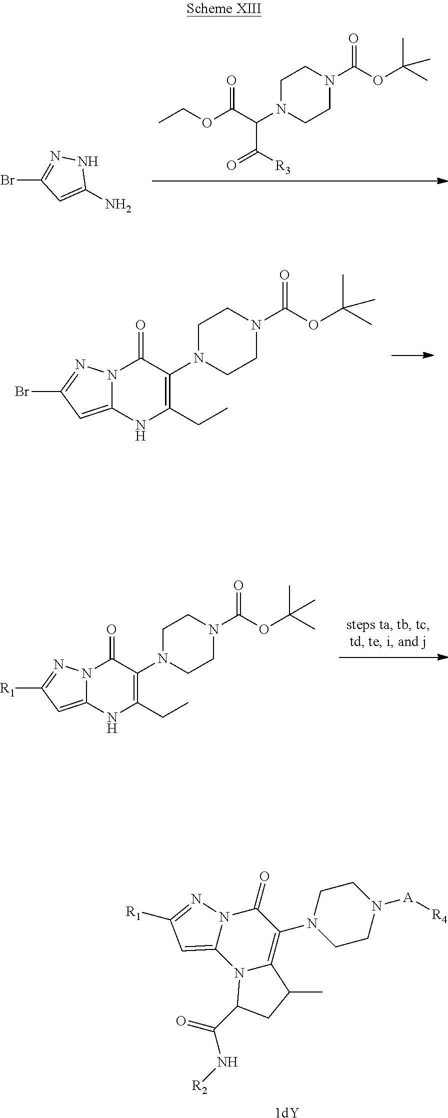

Compound of formula 1dY can be prepared according to the example route shown in Scheme XIII using analogous methods to those described in Scheme XI and analogous methods described herein. Analogous methods to chemists skilled in the art can be adapted accordingly. It is understood that the order of process steps shown in Scheme XIII may change to optimize the synthesis as necessary.

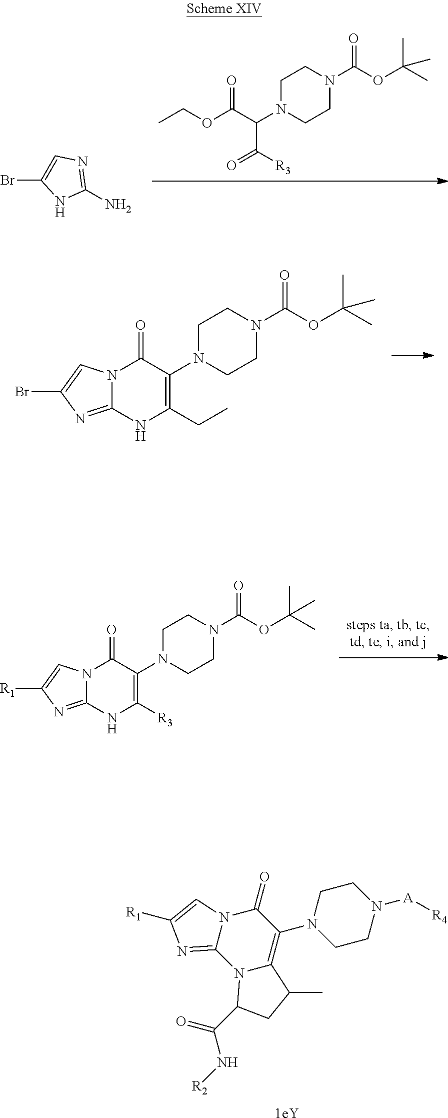

Compound of formula 1eY can be prepared according to the example route shown in Scheme XIII using analogous methods to those described in Scheme XI and analogous methods described herein. Analogous methods to chemists skilled in the art can be adapted accordingly. It is understood that the order of process steps shown in Scheme XIII may change to optimize the synthesis as necessary.

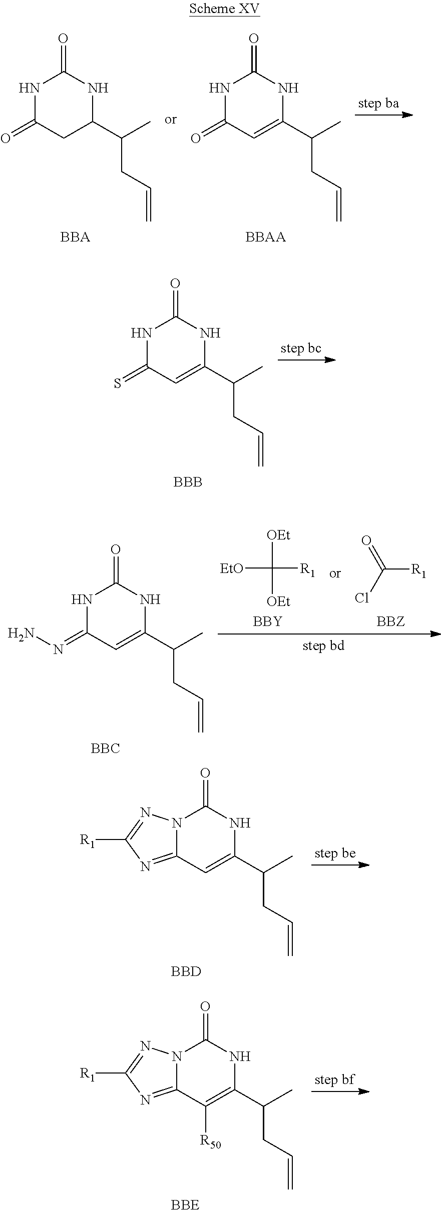

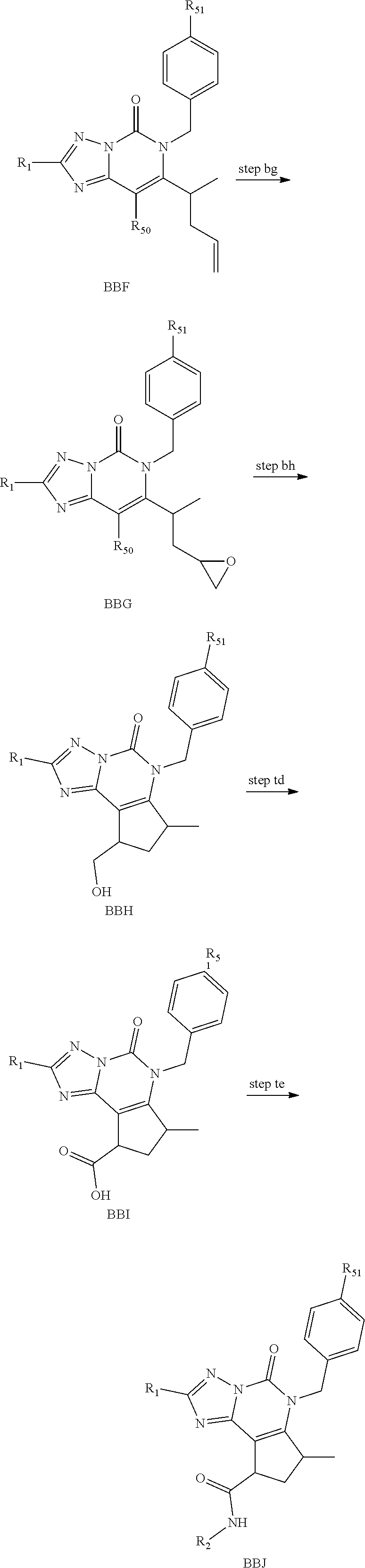

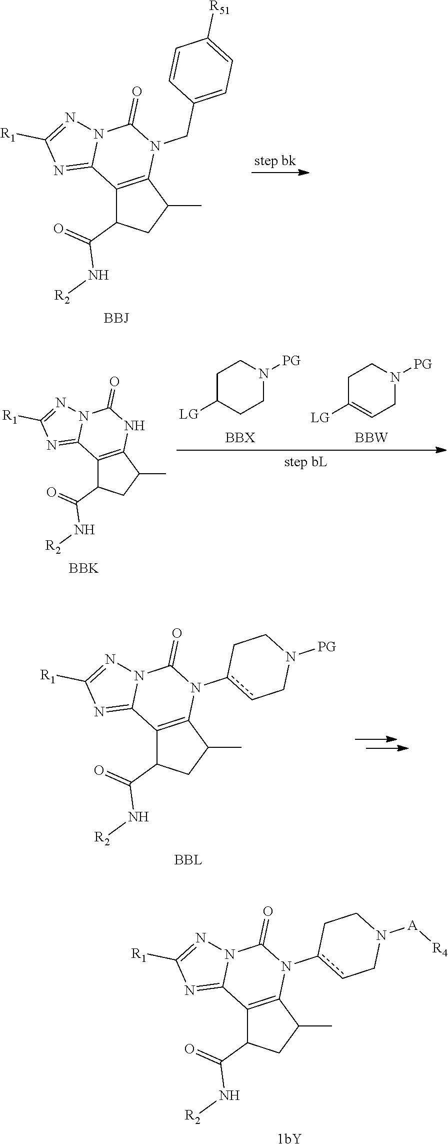

A process for preparing compound of formula BBL (Scheme XV) comprising steps ba, bc, bd, be, bf, bg, bh, td, te, bk and bL. Compound of formula BBL can subsequently be converted to compound of formula 1bY by methods analogous to those described herein. It is understood that the order of process ba, bc, bd, be, bf, bg, bh, td, te, bk and bL may change to optimize the synthesis as necessary.

Compound of formula BBL can be prepared comprising step bL starting from compound BBK wherein the substituents are as defined above with either compound BBX or compound BBW wherein PG is as defined above and LG is represented by halo, particularly iodo or bromo or OH or OMs (methanesulfonate) or OTs (p-toluenesulfonate) or OTf (trifluoromethanesulfonate) or B(OH)₂, BPin (Pin represents boronic acid pinacol ester) BF₃K. Step bL can be performed by combining compound of formula BBK with compound BBX in the presence of a base such as sodium hydride or K₂CO₃ or DBU or NaOtBu or phosphazene base P2-Et. A stoichiometric excess BBX can be used, preferably between 2 and 50 mole equivalents in an organic solvent for example DMF or NMP. The reaction is preferably stirred at a temperature of approximately 80-150° C. and can be carried out in a capped tube. An alternative method for step bL can use Ullmann-type reaction. Example Ullmann-type cross-coupling conditions comprise reacting compound of formula BBK with compound BBW in the presence of a catalyst such as copper(I)iodide, a ligand such as N-(2-cyanophenyl)pyridine-2-carboxamide or 4,7-dimethoxy-1,10-phenanthroline or N1,N2-dibenzylethane-1,2-diamine and a base such as K₃PO₄ or K₂CO₃ in a suitable solvent mixture such as DMSO or DMF at an approximate temperature of 80-150° C. A stoichiometric excess BBX can be used, preferably between 2 and 50 mole equivalents. An alternative method for step bL can comprise reacting compound of formula BBK with compound BBW using Buchwald-Hartwig conditions using for example using an analogous method to those described for step h (Scheme XI). Alternative cross-coupling conditions are known in the art, for examples of methods, see De Meijere et al. Metal-Catalyzed Cross-Coupling Reactions, Wiley, 2014 and references cited therein.

Compound of formula BBK can be prepared comprising step bk starting from compound of formula BBJ and the substituents are as defined above. Step bk comprises reacting a compound of formula BBJ with a stoichiometric excess of L-methionine for example 3 to 5 mol equivalents in a solvent such as methanesulfonic acid at an approximate temperature of 20-80° C.

Compound of formula BBJ can be prepared comprising steps td and te starting from compound of formula BBH wherein the substituents are as defined above using methods analogous to those described herein.

Compound of formula BBH can be prepared comprising step bh starting from compound of formula BBG wherein the substituents are as defined above. Step bh comprises reacting a compound of formula BBG with a base such as n-butyllithium or methyllithium or tert-butyllithium in a solvent such as THF at an approximate temperature of −100 to 0° C. Alternatively, compound of formula BBG can be treated with a base such as isopropylmagnesium chloride lithium chloride complex in a solvent such as THF at an approximate temperature of −78 to 25° C.

Compound of formula BBG can be prepared comprising steps bg starting from compound of formula BBF wherein the substituents are as defined above using methods analogous to those described herein.

Compound of formula BBF can be prepared comprising step bf starting from compound of formula BBE wherein the substituents are as defined above. Step bf comprises reacting a compound of formula BBE with a base such as sodium hydride at an approximate temperature of 0-20° C. in a solvent such as DMF for approximately 5 to 30 minutes under an inert gas such as nitrogen or argon. A compound such as 1-(bromomethyl)-4-methoxybenzene is then added and the reaction is stirred at a temperature of approximately 0-20° C.

Compound of formula BBE can be prepared comprising step be wherein compound of formula BBD wherein the substituents are defined as above is halogenated. Step be can be carried out using a halogenating reagent such as N-bromosuccinimide or N-iodosuccinimide in a solvent for example DMF or acetonitrile. The reaction is preferably stirred at a temperature of approximately 20-80° C.

Compound of formula BBD can be prepared comprising step bd starting from compound of formula BBC wherein the substituents are as defined above. Step bd comprises reacting a compound of formula BBC with a compound of formula BBY wherein R₁ is defined as above in a solvent such as DMF or toluene or dioxane at a temperature of approximately 80-150° C. An alternative method for step bd can comprise reacting compound of formula BBC with a compound of formula BBZ wherein R₁ is defined as above in a solvent such as dichloroethane or DMF or toluene or dioxane at a temperature of approximately 0-20° C. A base such as triethylamine may be added. The reaction is then stirred at an approximate temperature of 80-150° C.

Compound of formula BBC can be prepared comprising step bc starting from compound of formula BBB wherein the substituents are defined as above. Step bc comprises reacting a compound of formula BBB with a stoichiometric excess of hydrazine hydrate for example 2 to 5 mol equivalents in a solvent such as ethanol. The reaction is preferably stirred at a temperature of approximately 60-100° C.

Compound of formula BBB can be prepared comprising step ba starting from compound of formula BBA or BBAA wherein the substituents are defined as above. Methods for the preparation of compound of formula BBA or BBAA are known in the art. Step ba comprises reacting a compound of formula BBA or BBAA with P2S5 or lawessons reagent in a solvent such as dioxane or pyridine. The reaction is preferably stirred at a temperature of approximately 80-120° C.

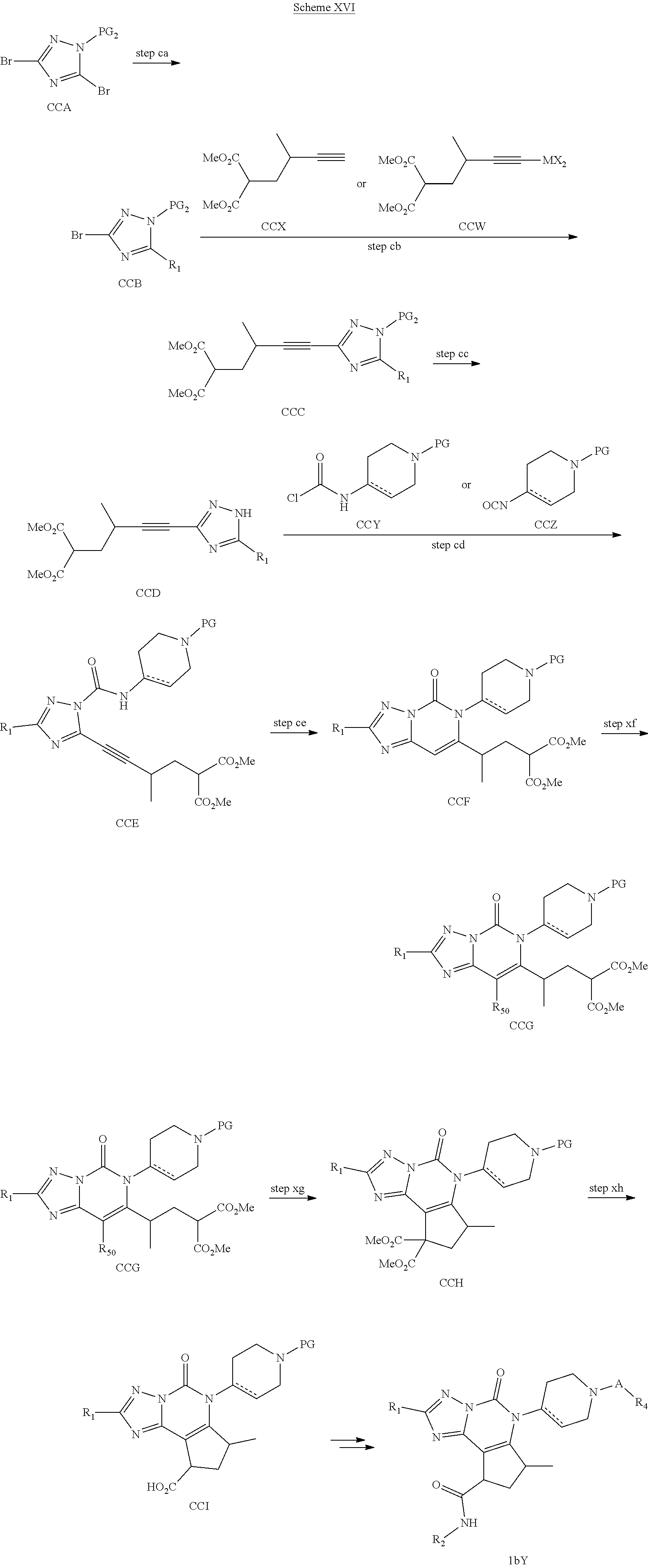

Compound of formula CCI can be prepared according to the route shown in Scheme XVI comprising steps ca, cb, cc, cd, ce, xf, xg and xh. Compound of formula CCI can subsequently be converted to compound of formula 1bY by methods analogous to those described herein. It is understood that the order of process ca, cb, cc, cd, ce, xf, xg and xh. may change to optimize the synthesis as necessary. Methods comprising steps xg, xh, xi, xj, xk, xe or xi and xj to prepare compounds of formulas CCG, CCH, CCI, CCJ, CCL, CCM and CCN can be performed using analogous conditions as those described above for steps bf, bg, bh, bi, e, i and j for Scheme XI and Scheme XV.

Compound of formula CCI can be prepared comprising step xh starting from compound of formula CCH wherein the substituents are defined as above. Step xh comprises reacting a compound of formula CCH with 6M HCl (aq) or concentrated HCl (aq) at a temperature of approximately 40-100° C. Alternatively, compound of formula CCH can be treated with excess sodium hydroxide at a temperature of approximately 20-50° C. in a solvent such as dimethylacetamide and subsequently treated with 6M HCl (aq) or concentrated HCl (aq) to obtain a pH 1-4 and stirred at a temperature of approximately 40-100° C.

Compound of formula CCH can be prepared comprising step xg starting from compound of formula CCG wherein the substituents are defined as above. Step xg comprises reacting a compound of formula CCG with a catalyst such as copperI) iodide in the presence of a ligand such as 2-picolinic acid and a base such as cesium carbonate in a solvent such as dimethylacetamide or THF at a temperature of approximately 40-120° C. Water (1 stoichiometric equivalent may be optionally added. Alternatively, step xg comprises reacting a compound of formula CCG with a catalyst such as Pd(PPh₃)₄ or XPhos Pd G4 in the presence of a base such as cesium carbonate or sodium hydride in a solvent such as dimethylacetamide or THF at a temperature of approximately 40-140° C.

Compound of formula CCG can be prepared comprising step xf wherein compound of formula CCF wherein the substituents are defined as above is halogenated. Step xf can be carried out using a halogenating reagent such as N-bromosuccinimide or N-iodosuccinimide in a solvent for example DMF or acetonitrile. The reaction is preferably stirred at a temperature of approximately 20-80° C.

Compound of formula CCF wherein PG represents a suitable protecting group such as BOC or para-methoxybenzyl or benzyl and the other substituents are as defined above can be prepared comprising step ce starting from compound of formula CCE wherein substituents are as defined above. Step ce comprises reacting a compound of formula CCE with Echavarren's gold(I) catalyst in a solvent such as THF at a temperature of approximately 60-140° C., preferably 80-120° C. in a sealed tube. An example method is described in Org. Lett. 2013, 15, 11, 2616-2619. An alternative method of preparing compound of formula CCF comprises reacting compound of formula CCE with a base such as sodium hydride in a solvent such as DMF or THF or dioxane at a temperature of approximately 60-140° C., preferably 80-120° C. under an inert gas such as nitrogen or argon in a sealed tube.

Compound of formula CCE can be prepared comprising step cd starting from compound CCD wherein the substituents are defined as above. Step cd comprises reacting a compound of formula CCD with compound of formula CCY or CCZ wherein PG represents a suitable protecting group such as BOC or para-methoxybenzyl or benzyl in the presence of a base such as triethylamine or N-ethyl-N,N-diisopropylamine in a solvent such as THF or dioxane or DMF at a temperature of approximately 20-140° C. preferably 60-120° C. Compound of formula CCY or CCZ are commercially available, or methods for their preparation are known in the art.

Compound of formula CCD can be prepared comprising step cc starting from compound CCC wherein PG2 represents a protecting group such as MOM (methoxymethyl) or SEM (trimethylsilyl)ethoxymethyl) and the other substituents are as defined above. Step cc comprises reacting a compound of formula CCC with an acid such as HCl or TFA in a solvent such as dioxane or dichloromethane at a temperature of approximately 0-80° C. preferably 20-60° C. Alternative methods for deprotection of SEM or MOM groups are known in the art.

Compound of formula CCC can be prepared comprising step cb starting from compound of formula CCB wherein the substituents are as defined above. Step cb can be a Sonogashira reaction reacting a compound of formula CCB with a compound of formula CCX wherein R₃ is as defined above in the presence of a catalyst such as Pd(PPh3)4 and a copper catalyst such as copper(I) iodide and a base such as triethylamine or lithium carbonate in a solvent such as dioxane or DMF or acetonitrile THF at a temperature of approximately 20-120° C., preferably 80-120° C. under an inert gas such as nitrogen or argon. Step cb can alternatively be a Suzuki or Stille cross-coupling reaction and comprises reacting a compound of formula CCB with a compound of formula CCW wherein MX2 represents B(OH)₂, BPin (Pin represents boronic acid pinacol ester) BF₃K, B(MIDA), tributyltin and R₃ is as defined above. Methods for Sonogashira, Suzuki or Stille reactions are known in the art. for examples of methods, see Molnar et al. Palladium-Catalyzed Coupling Reactions, Wiley, 2013 and references cited therein.

Compound of formula CCB can be prepared comprising step ca starting from compound of formula CCA wherein the substituents are defined as above. Step a can be a Suzuki cross-coupling reaction and comprises reacting a compound of formula CCA with R₁n-MX wherein R₁ is as defined above, n is 1, 2, 3 or 4 and MX represents for example B(OH)₂, BPin (Pin represents boronic acid pinacol ester) BF₃K. Example Suzuki cross-coupling conditions comprise reacting compound of formula CCA with R₁-BPin in the presence of a catalyst such as PdCl₂(dppf) or Pd(PPh₃)₄ and a base such as K₃PO₄ or potassium carbonate in a suitable solvent mixture such as DMF, THF or dioxane or water at a temperature of approximately 20-120° C., preferably 60-120° C. under an inert gas such as nitrogen or argon. Compound of formula CCA are commercially available, or methods for their preparation are known in the art.

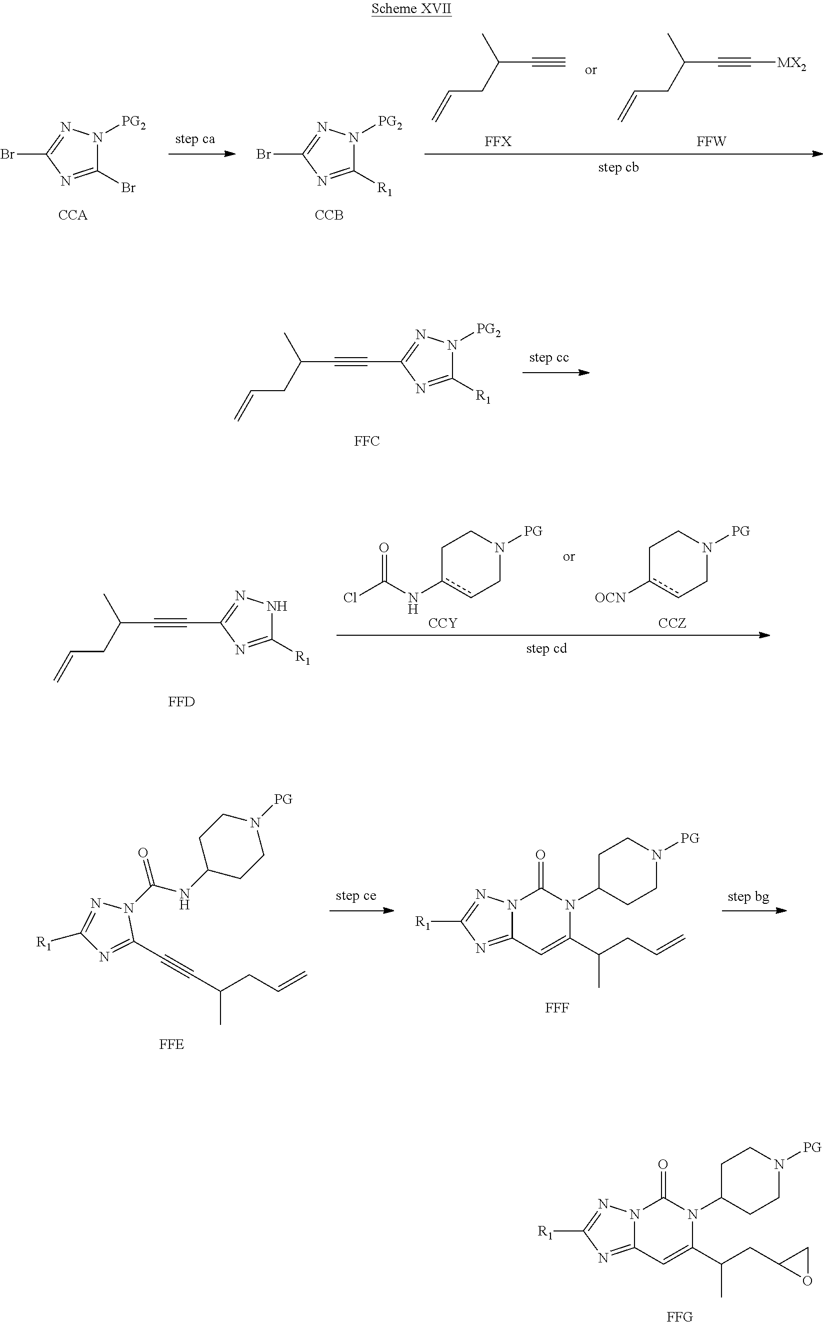

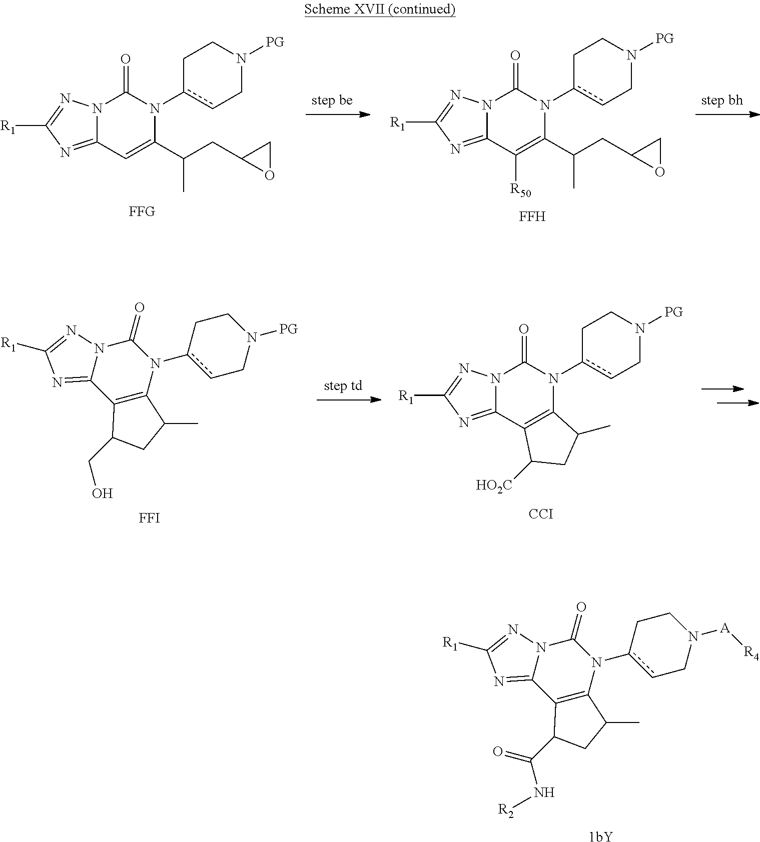

Alternatively, compound of formula CCI can be prepared according to the route shown in Scheme XVII comprising steps ca, cb, cc, cd, ce, bg, be, bh and bi using analogous conditions as those described in Scheme XV and XVI and herein. It is understood that the order of process ca, cb, cc, cd, ce, bg, be, bh and bi may change to optimize the synthesis as necessary.

Compound of formula FFC can be prepared by reacting compound of formula CCB with compound of formula FFX or FFW. Methods for their preparation of compounds of formula FFX and FFW are known in the art.

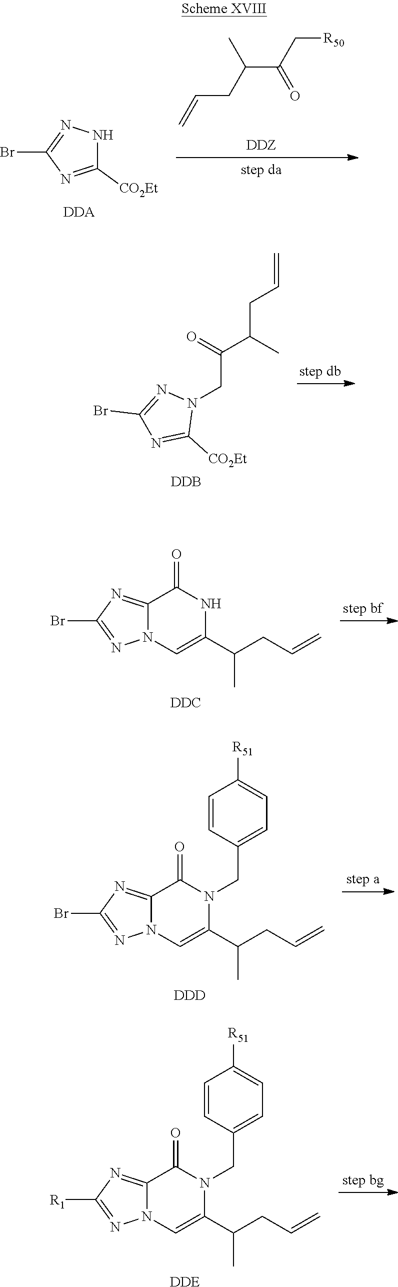

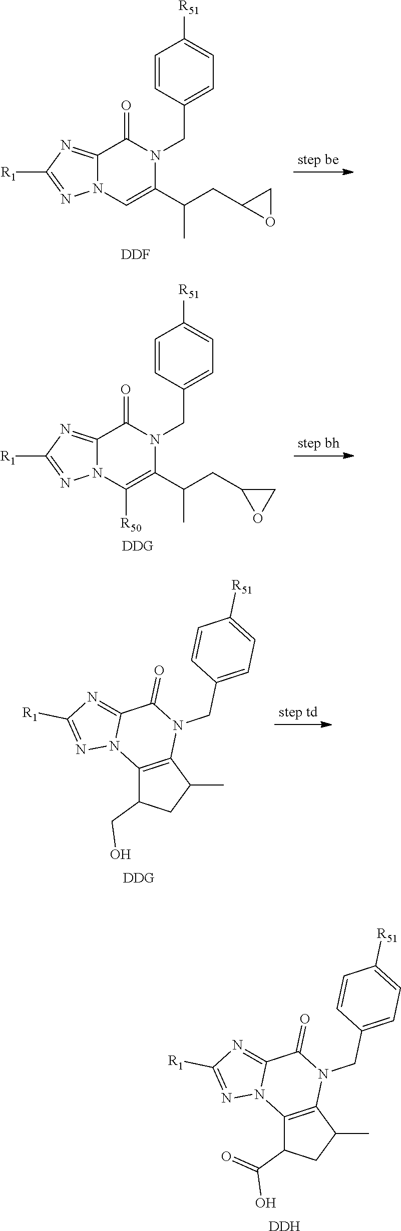

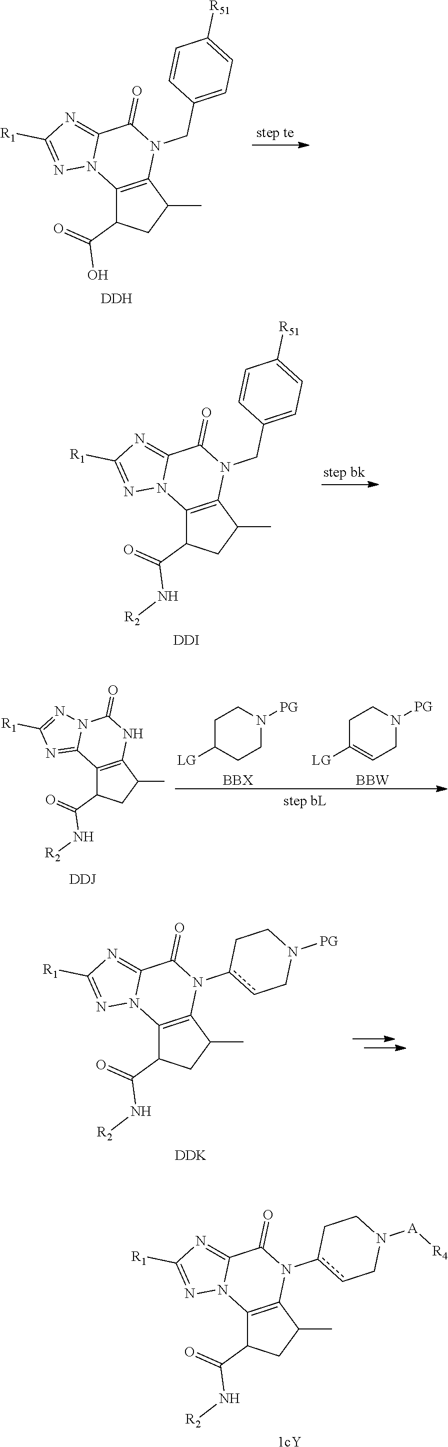

Compound of formula DDK can be prepared according to the route shown in Scheme XVIII comprising steps da, db, bf, a, bg, be, bh, td, te, bk and bL. Compound of formula DDK can subsequently be converted to compound of formula 1cY by methods analogous to those described herein. It is understood that the order of process da, db, bf, a, bg, be, bh, td, te, bk and bL. may change to optimize the synthesis as necessary.

Compound of formula 000 wherein the substituents are as defined above can be prepared comprising step db starting from compound of formula DDB wherein the substituents are as defined above. Step db comprises reacting compound of formula DDB with a stoichiometric excess of an ammonium salt such as ammonium acetate for example 3 to 100 mol equivalents particularly 10 to 20 mol equivalents in a solvent such as acetic acid at an approximate temperature of 60-130° C., preferably 80-120° C.

Compound of formula DDB wherein the substituents are as defined above can be prepared comprising step da starting from compound of formula DDA. Step da comprises reacting compound of formula DDA with compound of formula DDZ wherein R₅₀ represents halo particularly chloro or bromo and the other the substituents are as defined above in the presence of a base such as potassium carbonate in a solvent such as acetone or acetonitrile at an approximate temperature of 0-50° C., preferably 0-20° C. Methods for the preparation of compound of formula DDZ are known in the art.

### “Protecting Group”:

In the methods describe above, functional groups which are present in the starting materials and are not intended to take part in the reaction, are present in protected form if necessary, and protecting groups that are present are cleaved, whereby said starting compounds may also exist in the form of salts provided that a salt-forming group is present and a reaction in salt form is possible. In additional process steps, carried out as desired, functional groups of the starting compounds which should not take part in the reaction may be present in unprotected form or may be protected for example by one or more protecting groups. The protecting groups are then wholly or partly removed according to one of the known methods. Protecting groups, and the manner in which they are introduced and removed are described, for example, in “Protective Groups in Organic Chemistry”, Plenum Press, London, New York 1973, and in “Methoden der organischen Chemie”, Houben-Weyl, 4th edition, Vol. 15/1, Georg-Thieme-Verlag, Stuttgart 1974 and in Theodora W. Greene, “Protective Groups in Organic Synthesis”, John Wiley & Sons, New York 1981. A characteristic of protecting groups is that they can be removed readily, i.e. without the occurrence of undesired secondary reactions, for example by solvolysis, reduction, photolysis or alternatively under physiological conditions

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure antipodes.

Compounds of the invention and intermediates can also be converted into each other according to methods generally known to those skilled in the art.

Intermediates and final products can be worked up and/or purified according to standard methods, e.g. using chromatographic methods, distribution methods, (re-) crystallization, and the like.

The following applies in general to all processes mentioned herein before and hereinafter. All the above-mentioned process steps can be carried out under reaction conditions that are known to those skilled in the art, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g. in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers, for example analogously to the methods described herein above.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, methycyclohexane, or mixtures of those solvents, for example aqueous solutions, unless otherwise indicated in the description of the processes. Such solvent mixtures may also be used in working up, for example by chromatography or partitioning.

1. A compound of formula (I) or a pharmaceutically acceptable salt thereof: 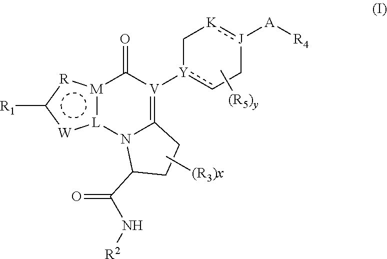
wherein
R, M, W, L, V and T are independently selected from C, CH and N,
to form subformulae 1a, 1b, 1c, 1d, 1e and 1f: 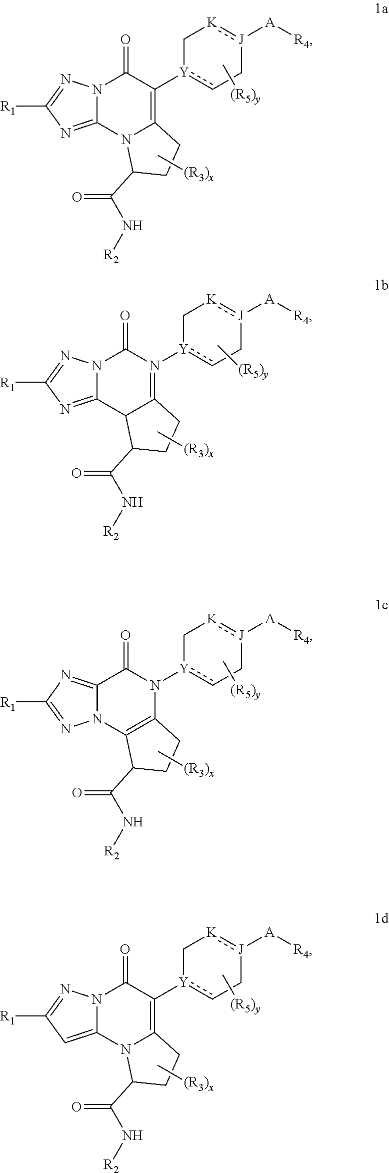 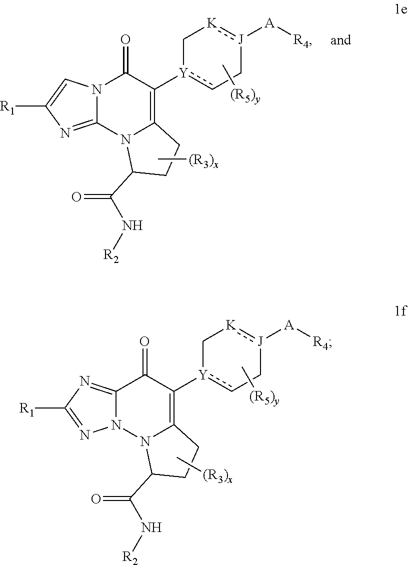
A is a linker selected from —C(O)—, —S(O)—, —S(O)₂—, and 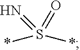
Y is N, C or CH;
Y [symbol] means Y is linked via a single bond to the adjacent carbon atom when Y is CH, or Y is linked via a double bond to the adjacent atom when Y is C, and when Y [symbol] is a single bond, Y is carbon unsubstituted or substituted by OH or F;
when Y is N, Y [symbol] is a single bond;
K [symbol] means K is linked via a single or double bond to the adjacent atom;
wherein:
when K [symbol] is a double bond, Y [symbol] is a single bond, K is CH, J is C, and A is a linker selected from —C(O)—, —S(O)—, —S(O)₂—, and 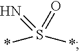
or
when K [symbol] is a single bond, K is selected from —CH₂—, —CH₂CH₂—, —NH— and a bond (to form a 5-membered ring: 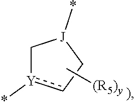
J is N, and A is a linker selected from —C(O)—, —S(O)—, —S(O)₂—, and 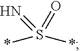
or
when K [symbol] is a single bond, K is —CH₂—, J is CH, and A is a linker selected from —S(O)—, —S(O)₂—, and 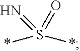
y is 0, 1, 2, 3 or 4;
R₅ is independently selected from:
—(C₁-C₄)alkyl,
—(C₃-C₅)cycloalkyl,
and wherein two R₅ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S,
when K [symbol] J is a carbon-nitrogen single bond, a R₅ substituent on K and on the adjacent carbon atom may Jon to form ring C: 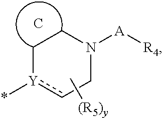
wherein ring C is a fused (C₃-C₆)cycloalkyl ring, a fused (C₃-C₆)heterocyclyl ring or a fused phenyl ring, wherein said fused (C₃-C₆)heterocyclyl ring contains ring carbon atoms and one ring heteroatom selected from O, N and S,
and wherein when ring C is a fused (C₃-C₆)cycloalkyl ring, said fused (C₃-C₆)cycloalkyl ring is unsubstituted or substituted with 1 or 2 R₄₀ groups, wherein said R₄₀ is selected from:
(C₁-C₂)alkyl, wherein each (C₁-C₂)alkyl is independently unsubstituted or substituted by OH or 1, 2 or 3 halo,
halo,
or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
or wherein two R₄₀ substituents on adjacent carbon atoms join together with the carbon atoms to which they are attached, to form a fused cyclopropyl ring;
when K [symbol] J is a carbon-carbon single bond, Y is N and Y [symbol] is a single bond, and A is a linker selected from —S(O)—, —S(O)₂—, and 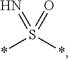
a R₅ substituent on K and on the adjacent carbon atom may join to form ring C: 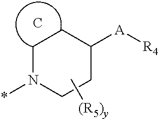
wherein ring C is a fused (C₃-C₆)cycloalkyl ring, a fused (C₃-C₆)heterocyclyl ring or a fused phenyl ring, wherein said fused (C₃-C₆)heterocyclyl ring contains ring carbon atoms and one ring heteroatom selected from O, N and S,
and wherein when ring C is a fused (C₃-C₆)cycloalkyl ring, said fused (C₃-C₆)cycloalkyl ring is unsubstituted or substituted with 1 or 2 R₄₀ groups, wherein said R₄₀ is selected from:
(C₁-C₂)alkyl, wherein each (C₁-C₂)alkyl is independently unsubstituted or substituted by OH or 1, 2 or 3 halo,
halo,
or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
or wherein two R₄₀ substituents on adjacent carbon atoms join together with the carbon atoms to which they are attached, to form a fused cyclopropyl ring;
and wherein when K is —CH₂— and J is N, two R₅ substituents may join to form a (C₁-C₃)alkylene bridge or a heteroalkylene bridge, wherein said heteroalkylene bridge is one heteroatom selected from N and O, or is —CH₂—O—CH₂—;
R₁ is:
cycloalkenyl, wherein said cycloalkenyl is a partially unsaturated monocyclic ring containing 5 or 6 ring carbon atoms, and said cycloalkenyl is unsubstituted or substituted by 1, 2, 3 or 4, R₃₃, wherein R₃₃ is halo, and wherein said cycloalkenyl or halo-substituted cycloalkenyl is substituted by 0, 1 or 2 R₁₅ substituents,
or R₁ is heterocyclyl, wherein said heterocyclyl is a 5 or 6 membered fully saturated or partially unsaturated group comprising ring carbon atoms and 1 or 2 ring heteroatoms independently selected from N, NH, O and S, and wherein said heterocyclyl is unbridged or bridged, and said bridge is 1 or 2 carbon atoms, wherein said heterocyclyl is unsubstituted or substituted by 1, 2, 3 or 4, R₃₃, wherein R₃₃ is halo, and wherein said heterocyclyl or halo-substituted heterocyclyl is substituted by 0, 1 or 2 substituents independently selected from R₁₅, R₁₆, R₁₇, R₁₈, R₁₉, R₂₀, R₂₂ and R₂₃,
or said heterocyclyl or halo-substituted heterocyclyl is fused to a cyclopropyl ring, wherein said cyclopropyl ring is unsubstituted or substituted by 1, 2 or 3 F,
or said heterocyclyl or halo-substituted heterocyclyl has 2 substituents at the same ring carbon atom which join to form a cyclopropyl spiro ring,
or said heterocyclyl or halo-substituted heterocyclyl is fused with a (C₃-C₅)heterocycloalkyl ring, wherein said (C₃-C₅)heterocycloalkyl ring contains ring carbon atoms and 1 ring O atom;
or R₁ is heteroaryl, wherein said heteroaryl is a 5 or 6 membered fully unsaturated monocyclic group comprising ring carbon atoms and 1, 2, 3 or 4 ring heteroatoms independently selected from N, O and S,
and wherein said heteroaryl is unsubstituted or substituted by 1, 2 or 3 substituents independently selected from R₂₁ and R₃₀, wherein R₂₁ and R₃₀ are independently selected from halo and (C₁-C₄)alkyl, wherein said (C₁-C₄)alkyl is unsubstituted or substituted by 1, 2 or 3 halo,
or R₁ is phenyl, wherein said phenyl is unsubstituted or substituted by 1, 2, 3 or 4, R₃₃, wherein R₃₃ is halo, and wherein said phenyl or halo-substituted phenyl is substituted by 0, 1 or 2 R₁₅ substituents,
or R₁ is (C₂-C₄)alkynyl or (C₂-C₄)alkenyl, wherein said (C₂-C₄)alkynyl and (C₂-C₄)alkenyl are unsubstituted or substituted by (C₁-C₄)alkyl-O—C(O)—, or morpholinyl;
each R₁₅, R₁₆, R₁₇, R₁₈, R₁₉, R₂₀, R₂₂ and R₂₃ is independently selected from:
halo
(C₁-C₄)alkyl-O— unsubstituted or substituted by 1, 2 or 3 halo;
(C₁-C₄)alkyl unsubstituted or substituted by OH, —O—(C₁-C₂)alkyl or 1, 2 or 3 halo,
HOC(O)—(CH₂)ₙ—,
H₃C—C(O)(CH₂)ₙ—,
(C₁-C₄)alkyl-O—C(O)(CH₂)ₙ,
═O
azetidinyl or pyrrolidinyl, wherein said azetidinyl and pyrrolidinyl are linked to the rest of the molecule via the N atom, and are each unsubstituted or substituted by 1 or 2 F,
R₂₅(R₂₄)N—, wherein R₂₄ is H or (C₁-C₄)alkyl unsubstituted or substituted by 1, 2 or 3 halo, R₂₅ is H or (C₁-C₄)alkyl unsubstituted or substituted by 1, 2 or 3 halo,
OH
wherein n is 0, 1 or 2,
R₂ is the moiety: 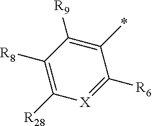
R₆ is selected from:
H,
halo,
(C₁-C₄)alkyl unsubstituted or substituted by 1, 2 or 3 halo,
(C₃-C₅)cycloalkyl unsubstituted or substituted by 1, 2 or 3 halo,
—O—(C₁-C₄)alkyl unsubstituted or substituted by 1, 2 or 3 halo,
OH, and
CN;
R₈ is selected from H, halo, and (C₁-C₄)alkyl unsubstituted or substituted by 1, 2 or 3 halo,
R₉ is selected from H, O—CH₃, OH, CN, CH₃ and halo;
R₂₈ is selected from:
SF₅,
H,
—C(O)H,
halo,
(C₁-C₄)alkyl unsubstituted or substituted by 1, 2 or 3 halo,
(C₁-C₄)alkynyl,
(C₁-C₄)alkenyl,
(C₃-C₅)cycloalkyl unsubstituted or substituted by 1, 2 or 3 halo, and
OCF₃;
X is selected from C—R₇ and N, wherein R₇ is H or halo, or R₇ can join, together with R₂₈ or R₆, and the atoms to which they are attached, to form a fused (C₄-C₆)cycloalkyl ring, wherein said fused (C₄-C₆)cycloalkyl ring is unsubstituted or substituted by 1, 2 or 3 halo,
or
R₂ is selected from: 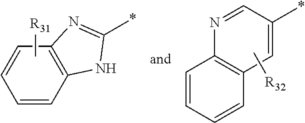
wherein
R₃₁ is selected from H, halo and CH₃,
R₃₂ is selected from H, halo and CH₃,
R₃ is selected from:
halo, and
(C₁-C₄)alkyl unsubstituted or substituted by 1, 2 or 3 substituents independently selected from halo and OH,
or two R₃ substituents on the same ring carbon atom may join, together with the carbon atom to which they are attached, to form a cyclopropyl ring;
x is 0, 1 or 2;
R₄ is selected from:
—(C₁-C₄)alkyl;
-heteroaryl1, wherein said heteroaryl1 is a 5 or 6 membered, fully unsaturated, monocyclic ring comprising ring carbon atoms and 1, 2, 3 or 4 ring heteroatoms independently selected from N, O and S;
-heteroaryl2, wherein said heteroaryl2 is a 9 or 10 membered fused bicyclic ring comprising ring carbon atoms and 1, 2, 3 or 4 ring heteroatoms independently selected from N, O and S, and wherein both rings are fully unsaturated, or one ring is fully unsaturated, and the other is saturated or partially unsaturated, and wherein the heteroatoms may be in one or both rings;
-phenyl;
wherein heteroaryl1, heteroaryl2 and phenyl are each substituted by 1, 2 or 3 substituents independently selected from R₁₀, R₁₁, R₁₂, R₁₃ and R₁₄, wherein each R₁₀, R₁₁, R₁₂, R₁₃ and R₁₄ is independently selected from:
H,
halo,
(C₁-C₄)alkyl unsubstituted or substituted by 1, 2 or 3 halo substituents,
(C₁-C₂)alkyl substituted by —O—(C₁-C₂)alkyl or OH,
—S—(C₁-C₃)alkyl,
—O—(C₁-C₄)alkyl unsubstituted or substituted by 1, 2 or 3 halo substituents,
OH,
(C₃-C₅)cycloalkyl, wherein said (C₃-C₅)cycloalkyl is unsubstituted or substituted by 1 or 2 halo,
—O—(C₃-C₅)cycloalkyl,
—NR₃₄R₃₅ wherein R₃₄ and R₃₅ are independently selected from:
H,
(C₁-C₄)alkyl, wherein said (C₁-C₄)alkyl is unsubstituted or substituted by OH or —O(C₁-C₂)alkyl,
and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
CN,
—(C₂-C₄)alkenyl,
—(C₂-C₄)alkynyl,
═O
—C(O)H, and
—C(O)(C₁-C₄)alkyl;
and * indicates a point of attachment.

2. A compound of formula (I) or a pharmaceutically acceptable salt thereof, according to claim 1, wherein the compound of formula (I) is formula 1a:

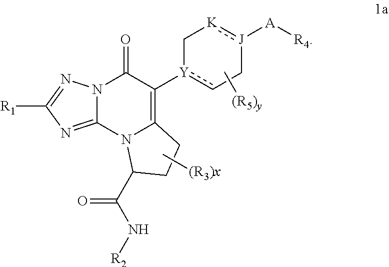

3. A compound of formula (I) or a pharmaceutically acceptable salt thereof, according to claim 1, wherein R₁ is:
cycloalkenyl, wherein said cycloalkenyl is a partially unsaturated monocyclic ring containing 5 or 6 ring carbon atoms, and said cycloalkenyl is unsubstituted or substituted by 1 or 2 R₃₃,
wherein R₃₃ is halo, and wherein said cycloalkenyl or halo-substituted cycloalkenyl is substituted by 0 or 1 R₁₅ substituents, wherein R₁₅ is selected from:
a) (C₁-C₂)alkyl-O— unsubstituted or substituted by 1, 2 or 3 halo;
b) (C₁-C₂)alkyl unsubstituted or substituted by 1, 2 or 3 halo,
c) HOC(O)—(CH₂)ₙ—,
d) H₃C—C(O)(CH₂)ₙ—,
e) H₃C—O—C(O)(CH₂)ₙ,
f)═O, and
g) R₂₅(R₂₄)N—, H, wherein R₂₄ is H or (C₁-C₂)alkyl unsubstituted or substituted by 1, 2 or 3 halo, R₂₅ is H or (C₁-C₂)alkyl unsubstituted or substituted by 1, 2 or 3 halo,
n is 0 or 1,
wherein
the R₁₅ substituent a) to g) of said cycloalkenyl or halo-substituted cycloalkenyl is not present on the ring atoms adjacent to the ring atom to which the cycloalkenyl or halo-substituted cycloalkenyl is joined to the remainder of the molecule, and
said cycloalkenyl or halo-substituted cycloalkenyl is linked to the remainder of the compound via a R₁ ring carbon atom which is double bonded to an adjacent R₁ ring carbon atom;
or R₁ is heterocyclyl, wherein said heterocyclyl is a 5 or 6 membered fully saturated or partially unsaturated group comprising ring carbon atoms and 1 or 2 ring heteroatoms independently selected from N, NH, O and S, and wherein said heterocyclyl is unbridged or bridged, and said bridge is 1 or 2 carbon atoms, wherein said heterocyclyl is unsubstituted or substituted by 1 or 2 R₃₃, wherein R₃₃ is halo, and wherein said heterocyclyl or halo-substituted heterocyclyl is substituted by 0 or 1 substituents independently selected from R₁₅, R₁₆, R₁₇, R₁₈, R₁₉, R₂₀, R₂₂ and R₂₃, wherein said R₁₅, R₁₆, R₁₇, R₁₈, R₁₉, R₂₀, R₂₂ and R₂₃ are independently selected from:
a) (C₁-C₄)alkyl-O— unsubstituted or substituted by 1, 2 or 3 halo;
b) (C₁-C₄)alkyl unsubstituted or substituted by OH, —O—(C₁-C₂)alkyl or 1, 2 or 3 halo,
c) HOC(O)—(CH₂)ₙ—,
d) H₃C—C(O)(CH₂)ₙ—,
e) H₃C—O—C(O)(CH₂)ₙ,
f)═O
g) R₂₅(R₂₄)N—, wherein R₂₄ is H, (C₁-C₂)alkyl unsubstituted or substituted by 1, 2 or 3 halo, R₂₅ is H, (C₁-C₂)alkyl unsubstituted or substituted by 1, 2 or 3 halo,
h) OH
wherein n is 0 or 1,
and wherein:
substituent a) to h) of said heterocyclyl or halo-substituted heterocyclyl is not present on the ring atoms adjacent to the ring atom to which the heterocyclyl or halo-substituted heterocyclyl is joined to the remainder of the molecule; and
said heterocyclyl is linked to the remainder of the compound via a R₁ ring nitrogen atom, or a R₁ ring carbon atom which is double bonded to an adjacent ring atom;
or R₁ is heteroaryl, wherein said heteroaryl is a 5 or 6 membered fully unsaturated monocyclic group comprising ring carbon atoms and 1 or 2 ring heteroatoms independently selected from N, O and S, wherein the total number of ring S atoms does not exceed 1, and the total number of ring O atoms does not exceed 1, wherein said heteroaryl is unsubstituted or substituted by 1 or 2 substituents independently selected from R₂₁ and R₃₀, wherein R₂₁ and R₃₀ are independently selected from (C₁-C₂)alkyl, and said (C₁-C₂)alkyl is unsubstituted or substituted by 1, 2 or 3 halo.

4. A compound of formula (I) or a pharmaceutically acceptable salt thereof, according to claim, wherein R₁ is selected from: 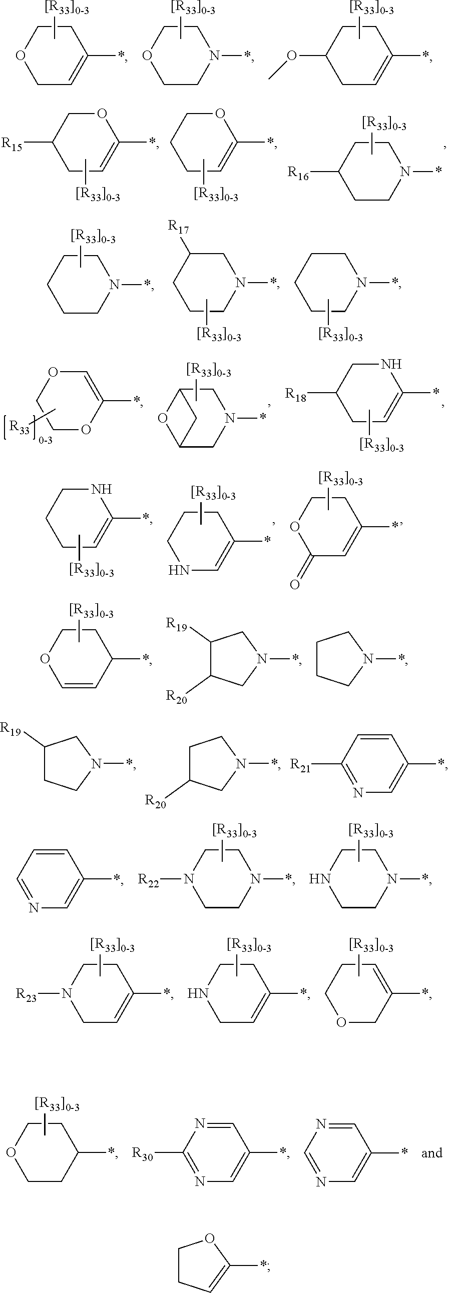
R₃₃ is F;
R₁₅ is halo, azetidinyl or pyrrolidinyl, wherein said azetidinyl and pyrrolidinyl are linked to the rest of the molecule via the N atom, and are unsubstituted or substituted by 1 or 2 F;
R₁₆ is R₂₅(R₂₄)N—, wherein R₂₄ is H or (C₁-C₂)alkyl, R₂₅ is H or (C₁-C₂)alkyl unsubstituted or substituted by 1, 2 or 3 halo;
R₁₇ is halo
R₁₈ is halo;
R₁₉ is halo;
R₂₀ is halo;
R₂₁ is (C₁-C₂)alkyl;
R₂₂ and R₂₃ are each independently selected from:
(C₁-C₄)alkyl unsubstituted or substituted by 1, 2 or 3 halo,
HOC(O)—(CH₂)ₙ—,
H₃C—C(O)(CH₂)ₙ—,
(H₃C)₃C—O—C(O)(CH₂)ₙ—;
wherein n is 0, 1 or 2;
and
R₃₀ is CH₃.

5. A compound of formula (I), or a pharmaceutically acceptable salt thereof, according to claim 1, wherein R₁ is selected from:

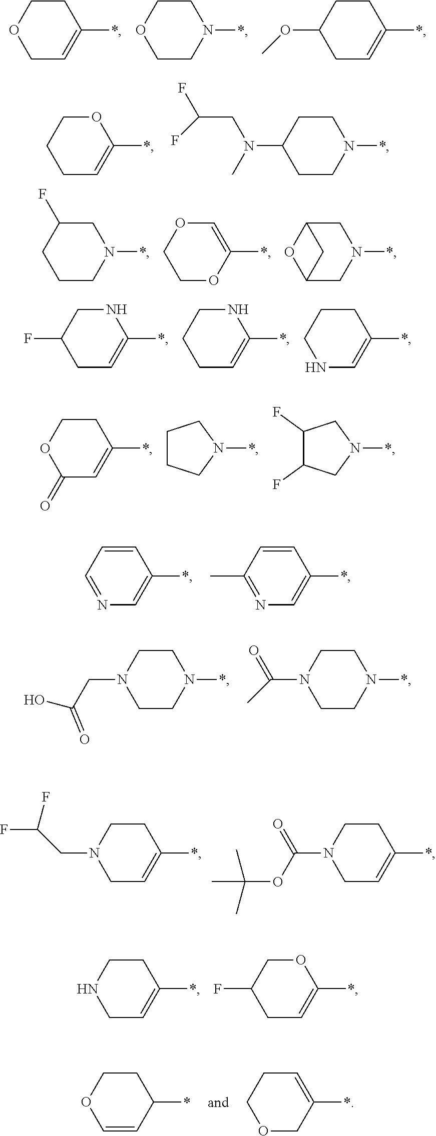

6. A compound of formula (I), or a pharmaceutically acceptable salt thereof, according to claim 1, wherein R₁ is selected from:

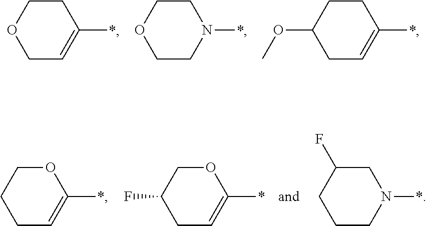

7. A compound of formula (I), or a pharmaceutically acceptable salt thereof, according to claim 1, wherein R₂ is the moiety: 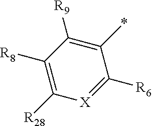
wherein
R₆ is selected from H, halo, (C₁-C₄)alkyl unsubstituted or substituted by 1, 2 or 3 halo;
R₈ is selected from H, halo, (C₁-C₄)alkyl unsubstituted or substituted by 1, 2 or 3 halo;
R₉ is selected from H, O—CH₃, OH, CN, CH₃ and halo;
R₂₈ is selected from SF₅, halo, C(O)H and (C₁-C₄)alkyl unsubstituted or substituted by 1, 2 or 3 halo;
X is selected from C—R₇ and N; and
R₇ is selected from H and halo.

8. A compound of formula (I), or a pharmaceutically acceptable salt thereof, according to claim 1, wherein R₂ is the moiety: 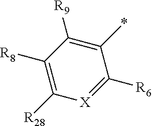
R₆ is selected from H, Cl, CH₃, F and Br;
R₈ is selected from H, Cl, F and CF₃;
R₉ is selected from H, CH₃ and Cl;
R₂₈ is selected from CF₃, CF₂H, —CH₂CH₃, Cl, SF₅, Br and —C(O)H;
X is selected from C—R₇ and N; and
R₇ is selected from H and F.

9. A compound of formula (I), or a pharmaceutically acceptable salt thereof, according to claim 1, wherein the moiety: 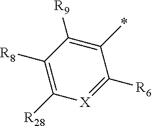
is selected from:

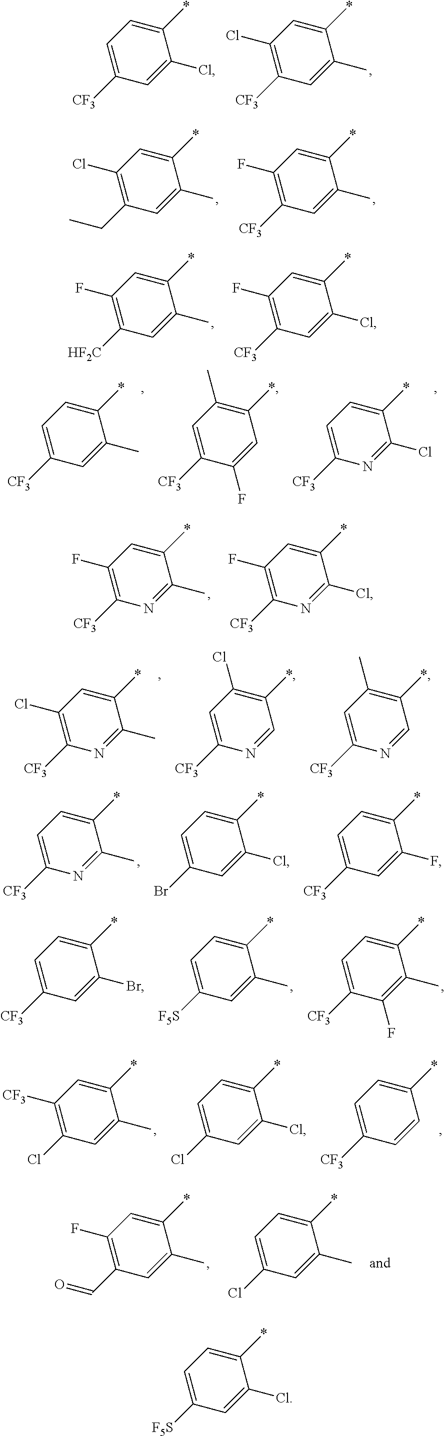

10. A compound of formula (I), or a pharmaceutically acceptable salt thereof, according to claim 1, wherein the moiety: 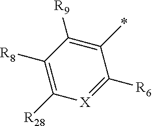
is selected from:

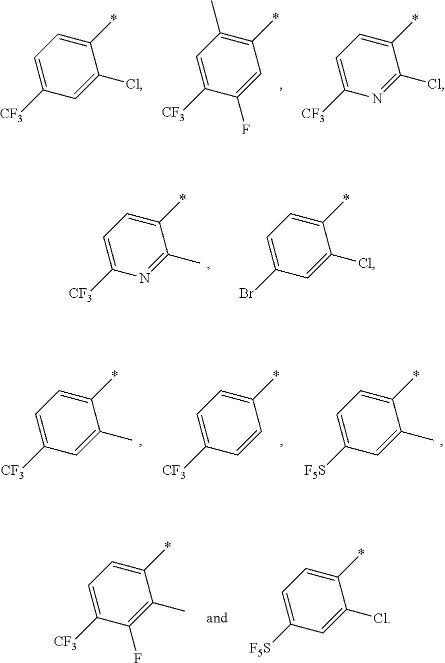

11. A compound of formula (I), or a pharmaceutically acceptable salt thereof, according to claim 1, wherein x is 0 or 1.

12. A compound of formula (I), or a pharmaceutically acceptable salt thereof, according to claim 1, wherein R₃ is (C₁-C₂)alkyl unsubstituted or substituted by 1, 2 or 3 substituents independently selected from halo and OH.

13. A compound of formula (I), or a pharmaceutically acceptable salt thereof, according to claim 1, wherein R₃ is CH₃.

14. A compound of formula (I), or a pharmaceutically acceptable salt thereof, according to claim 1, wherein Y is N and Y [symbol] is Y linked by a single bond.

15. A compound of formula (I), or a pharmaceutically acceptable salt thereof, according to claim 1, wherein K [symbol] is K linked by a single bond, K is —CH₂—, J is N, and A is a linker selected from —C(O)—, —S(O)—, —S(O)₂—, and

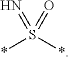

16. A compound of formula (I), or a pharmaceutically acceptable salt thereof, according to claim 1, wherein A is a —C(O)— linker.

17. A compound of formula (I), or a pharmaceutically acceptable salt thereof, according to claim 1, wherein R₅ is independently selected from:
—(C₁-C₄)alkyl,
and wherein two R₅ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S,
when K [symbol] J is a carbon-nitrogen single bond, a R₅ substituent on K and on the adjacent carbon atom may join to form ring C: 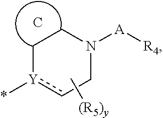
wherein ring C is a fused (C₃-C₆)cycloalkyl ring, a fused (C₃-C₆)heterocyclyl ring or a fused phenyl ring, wherein said fused (C₃-C₆)heterocyclyl ring contains ring carbon atoms and one ring heteroatom selected from O, N and S,
and wherein when ring C is a fused (C₃-C₆)cycloalkyl ring, said fused (C₃-C₆)cycloalkyl ring is unsubstituted or substituted with 1 or 2 R₄₀ groups, wherein said R₄₀ is selected from:
(C₁-C₂)alkyl, wherein each (C₁-C₂)alkyl is independently unsubstituted or substituted by OH or 1, 2 or 3 halo,
halo,
or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
or wherein two R₄₀ substituents on adjacent carbon atoms join together with the carbon atoms to which they are attached, to form a fused cyclopropyl ring;
and wherein when K is —CH₂— and J is N, two R₅ substituents may join to form a (C₁-C₃)alkylene bridge or a heteroalkylene bridge, wherein said heteroalkylene bridge is one heteroatom selected from N and O, or is —CH₂—O—CH₂—.

18. A compound of formula (I), or a pharmaceutically acceptable salt thereof, according to claim 1, wherein R₅ is independently selected from:
—(C₁-C₂)alkyl-preferably-methyl, and
when K [symbol] J is a carbon-nitrogen single bond, a R₅ substituent on K and on the adjacent carbon atom may join to form ring C: 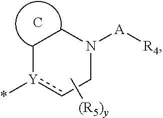
wherein ring C is a fused (C₃-C₄)cycloalkyl ring, and said fused (C₃-C₄)cycloalkyl ring, is unsubstituted or substituted with 1 or 2 R₄₀ groups, wherein said R₄₀ is selected from:
(C₁-C₂)alkyl, wherein each (C₁-C₂)alkyl is independently unsubstituted or substituted by OH or 1, 2 or 3 halo,
halo,
or wherein two R₄₀ substituents on the same ring carbon atom may join, together with the carbon atom to which they are attached, to form a (C₃-C₄)cycloalkyl spiro ring or a 3 or 4-membered heterocyclyl spiro ring, wherein said heterocyclyl spiro ring contains ring carbon ring atoms and one ring heteroatom selected from O, N and S;
or wherein two R₄₀ substituents on adjacent carbon atoms join together with the carbon atoms to which they are attached, to form a fused cyclopropyl ring;

19. A compound of formula (I) or a pharmaceutically acceptable salt thereof, according to claim 1, wherein R₅ is independently selected from:
CH₃, and y is 1 or 2, and
when K [symbol] J is a carbon-nitrogen single bond, a R₅ substituent on K and on the adjacent carbon atom may join to form ring C: 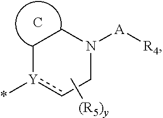
wherein ring C is a fused cyclobutyl ring.

20. A compound of formula (I) or a pharmaceutically acceptable salt thereof, according to claim 1, wherein the compound of formula (I) includes the moiety:

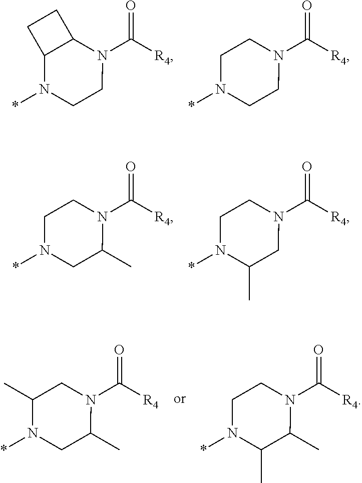

21. A compound of formula (I) or a pharmaceutically acceptable salt thereof, according to claim 1, wherein y is 0, 1, or 2.

22. A compound of formula (I) or a pharmaceutically acceptable salt thereof, according to claim 1, wherein R₄ is selected from:
—(C₁-C₄)alkyl;
-heteroaryl1, wherein said heteroaryl1 is a 5 or 6 membered, fully unsaturated monocyclic ring comprising ring carbon atoms and 1, 2, 3 or 4 ring heteroatoms independently selected from N, O and S, and the total number of ring S atoms does not exceed 1, and the total number of ring O atoms does not exceed 1;
-heteroaryl2, wherein said heteroaryl2 is a 9 or 10 membered fused bicyclic ring comprising ring carbon atoms and 1, 2, 3 or 4 ring heteroatoms independently selected from N, O and S, and wherein both rings are fully unsaturated, or one ring is fully unsaturated and the other is saturated or partially unsaturated, and wherein the heteroatoms may be in one or both rings, and the total number of ring S atoms does not exceed 1, and the total number of ring O atoms does not exceed 1;
-phenyl;
wherein heteroaryl1, heteroaryl2 and phenyl are each substituted by 1, 2 or 3 substituents independently selected from R₁₀, R₁₁, R₁₂, R₁₃ and R₁₄, wherein each R₁₀, R₁₁, R₁₂, R₁₃ and R₁₄ is independently selected from:
H,
halo,
(C₁-C₄)alkyl unsubstituted or substituted by 1, 2 or 3 halo substituents,
(C₁-C₂)alkyl substituted by —O—(C₁-C₂)alkyl or OH,
—S—(C₁-C₃)alkyl,
—O—(C₁-C₄)alkyl unsubstituted or substituted by 1, 2 or 3 halo substituents,
OH,
(C₃-C₅)cycloalkyl, wherein said (C₃-C₅)cycloalkyl is unsubstituted or substituted by 1 or 2 halo,
—O—(C₃-C₅)cycloalkyl,
—NR₃₄R₃₅ wherein R₃₄ and R₃₅ are independently selected from:
H,
(C₁-C₄)alkyl, wherein said (C₁-C₄)alkyl is unsubstituted or substituted by OH or —O(C₁-C₂)alkyl,
and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
CN,
—(C₂-C₄)alkenyl,
—(C₂-C₄)alkynyl,
═O
—C(O)H, and
—C(O)(C₁-C₄)alkyl;
with the proviso that one OH substituent is present on heteroaryl1, heteroaryl2 and phenyl.

23. A compound of formula (I) or a pharmaceutically acceptable salt thereof, according to claim 1, wherein R₄ is selected from:
—(C₁-C₄)alkyl;
-heteroaryl1;
-heteroaryl2; and
-phenyl;
wherein heteroaryl1, heteroaryl2 and phenyl are each substituted by 1, 2 or 3 substituents independently selected from R₁₀, R₁₁, R₁₂, R₁₃ and R₁₄, wherein each R₁₀, R₁₁, R₁₂, R₁₃ and R₁₄ is independently selected from:
H,
halo,
(C₁-C₄)alkyl unsubstituted or substituted by 1, 2 or 3 halo substituents,
(C₁-C₂)alkyl substituted by —O—(C₁-C₂)alkyl or OH,
—S—(C₁-C₃)alkyl,
—O—(C₁-C₄)alkyl unsubstituted or substituted by 1, 2 or 3 halo substituents,
OH,
(C₃-C₅)cycloalkyl, wherein said (C₃-C₅)cycloalkyl is unsubstituted or substituted by 1 or 2 halo,
—O—(C₃-C₅)cycloalkyl,
—NR₃₄R₃₅ wherein R₃₄ and R₃₅ are independently selected from:
H,
(C₁-C₄)alkyl, wherein said (C₁-C₄)alkyl is unsubstituted or substituted by OH or —O(C₁-C₂)alkyl,
and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
CN,
—(C₂-C₄)alkenyl,
—(C₂-C₄)alkynyl,
═O
—C(O)H, and
C(O)(C₁-C₄)alkyl;
with the proviso that:
one OH substituent is present on heteroaryl1, heteroaryl2 and phenyl, and said OH is in the ortho position of the R₄ ring, relative to the position linking R₄ to linker -A-,
or
one ═O substituent is present on heteroaryl1 and heteroaryl2.

24. A compound of formula (I) or a pharmaceutically acceptable salt thereof, according to claim 1, wherein R₄ is selected from:
—(C₁-C₄)alkyl; 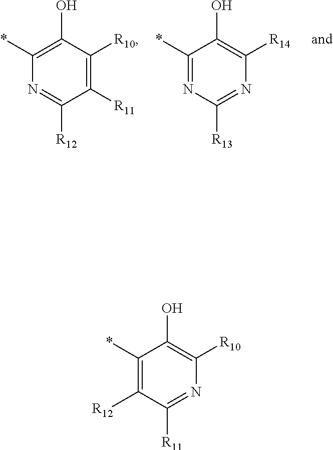
wherein
R₁₀, R₁₁, R₁₂, R₁₃ and R₁₄ are independently selected from:
H,
halo,
(C₁-C₄)alkyl unsubstituted or substituted by 1, 2 or 3 halo substituents,
(C₁-C₂)alkyl substituted by —O—(C₁-C₂)alkyl or OH,
—S—(C₁-C₃)alkyl,
—O—(C₁-C₄)alkyl unsubstituted or substituted by 1, 2 or 3 halo substituents,
(C₃-C₅)cycloalkyl, wherein said (C₃-C₅)cycloalkyl is unsubstituted or substituted by 1 or 2 halo,
—O—(C₃-C₅)cycloalkyl,
—NR₃₄R₃₅ wherein R₃₄ and R₃₅ are independently selected from:
H,
(C₁-C₄)alkyl, wherein said (C₁-C₄)alkyl is unsubstituted or substituted by OH or —O(C₁-C₂)alkyl,
and wherein R₃₄ and R₃₅ can join, together with the atom to which they are attached, to form an azetidine, pyrrolidinyl or piperidine ring, wherein said azetidine, pyrrolidinyl and piperidine are unsubstituted or substituted with CH₃;
CN,
—(C₂-C₄)alkenyl,
—(C₂-C₄)alkynyl,
—C(O)H, and
—C(O)(C₁-C₄)alkyl.

25. A compound of formula (I) or a pharmaceutically acceptable salt thereof, according to claim 1, wherein R₄ is selected from: 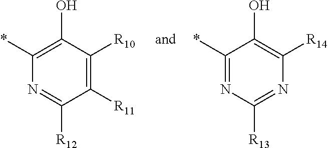
wherein
R₁₀ is selected from H, halo, (C₁-C₂)alkyl unsubstituted or substituted by 1, 2 or 3 halo substituents, —O—(C₁-C₂)alkyl unsubstituted or substituted by 1, 2 or 3 halo substituents;
R₁₁ is selected from H, halo, (C₁-C₂)alkyl unsubstituted or substituted by 1, 2 or 3 halo substituents;
R₁₂ is selected from H, halo, (C₁-C₂)alkyl unsubstituted or substituted by 1, 2 or 3 halo substituents;
R₁₃ is selected from H, —S—CH₃, halo, (C₁-C₂)alkyl unsubstituted or substituted by 1, 2 or 3 halo substituents; and
R₁₄ is selected from H, halo, (C₁-C₂)alkyl unsubstituted or substituted by 1, 2 or 3 halo substituents, O—(C₁-C₂)alkyl unsubstituted or substituted by 1, 2 or 3 halo substituents, and cyclopropyl.

26. A compound of formula (I) or a pharmaceutically acceptable salt thereof, according to claim 1, wherein R₄ is selected from:
—CH₃,

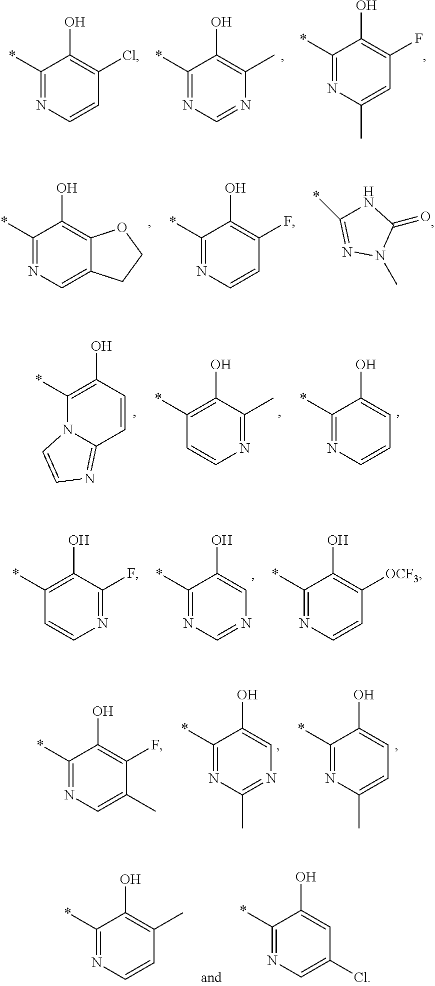

27. A compound of formula (I), or a pharmaceutically acceptable salt thereof, according to claim 1, wherein R₄ is selected from:

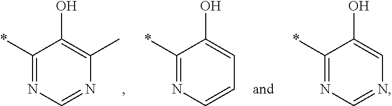

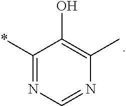 [SMILES: *c1ncnc(C)c1O]

28. A compound of formula (I), or a pharmaceutically acceptable salt thereof, according to claim 1, wherein the compound of formula (I) has the stereochemistry shown in formula (I′):

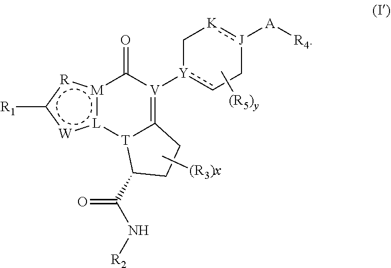

29. A compound of formula (I), or a pharmaceutically acceptable salt thereof, according to claim 1, wherein the compound of formula I′ has the formula I′″:

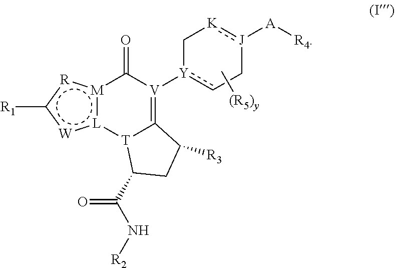

30. A compound of formula (I), or a pharmaceutically acceptable salt thereof, according to claim 1, wherein formula (I) is formula 1g, 1g′, 1g* or 1g**:

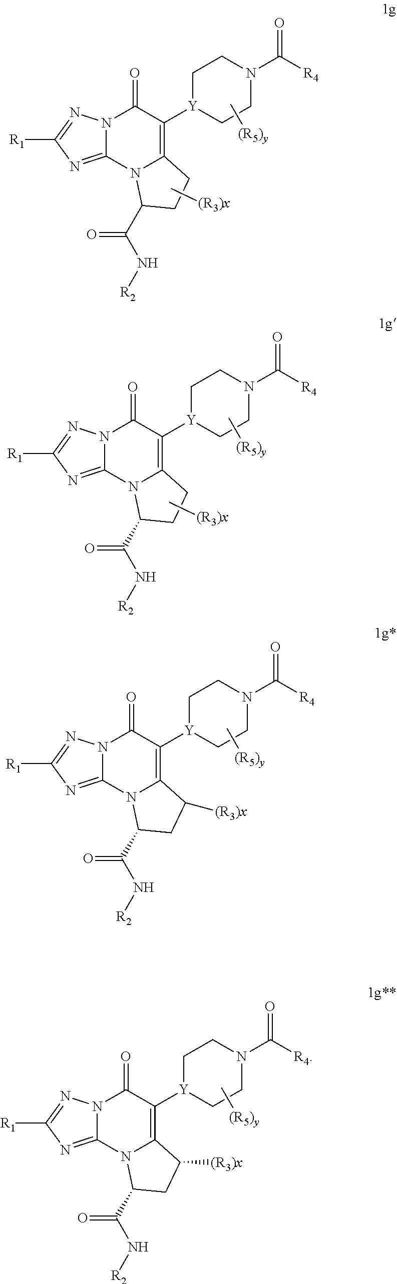

31. A compound of formula (I) or a pharmaceutically acceptable salt thereof, according to claim 1, wherein formula (I) is formula 1h or 1h′:

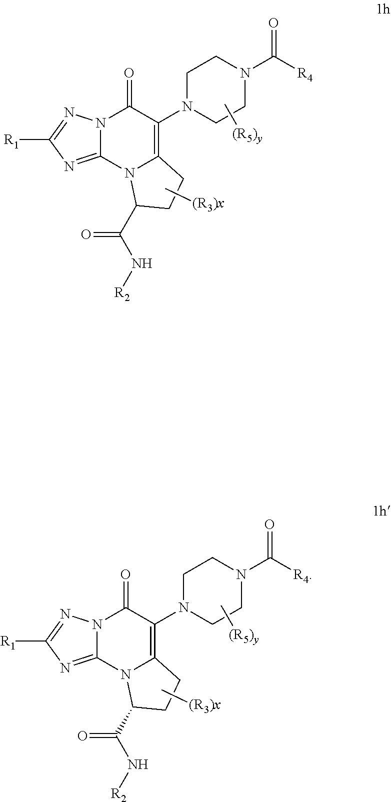

32. A compound of formula (I), or a pharmaceutically acceptable salt thereof, according to claim 1, wherein the compound is selected from:

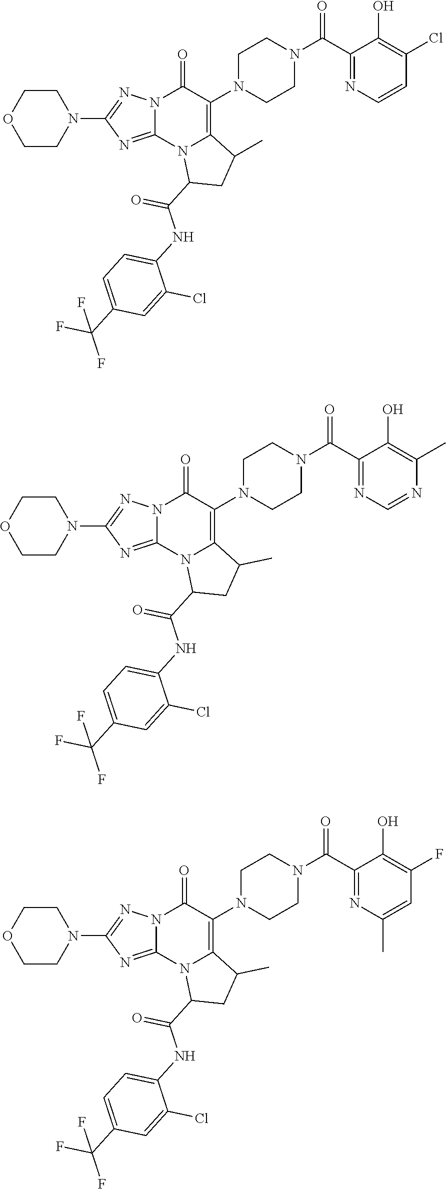 [SMILES: CC1CC(C(=O)Nc2ccc(C(F)(F)F)cc2Cl)n2c1c(N1CCN(C(=O)c3nccc(Cl)c3O)CC1)c(=O)n1nc(N3CCOCC3)nc21 ; Cc1ncnc(C(=O)N2CCN(c3c4n(c5nc(N6CCOCC6)nn5c3=O)C(C(=O)Nc3ccc(C(F)(F)F)cc3Cl)CC4C)CC2)c1O ; Cc1cc(F)c(O)c(C(=O)N2CCN(c3c4n(c5nc(N6CCOCC6)nn5c3=O)C(C(=O)Nc3ccc(C(F)(F)F)cc3Cl)CC4C)CC2)n1]

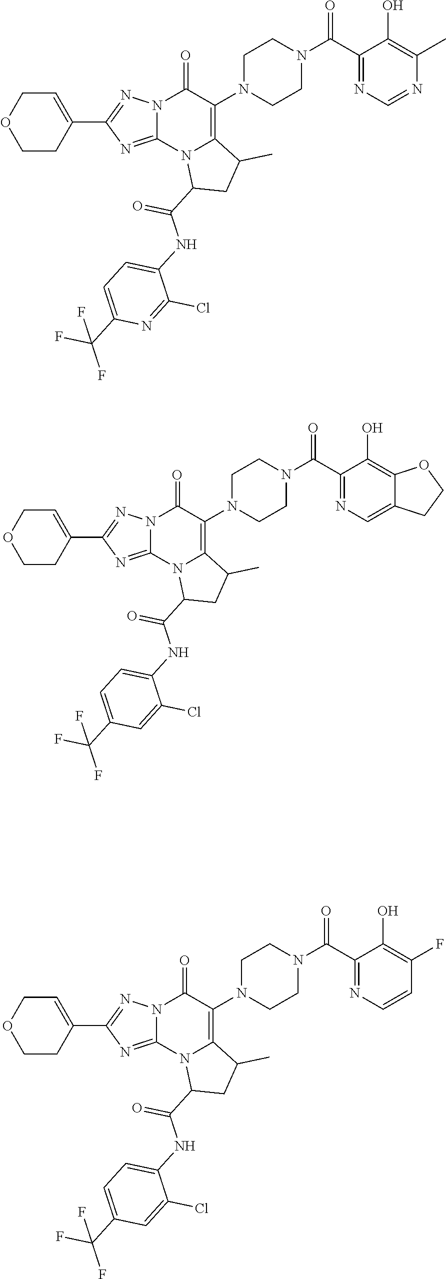 [SMILES: Cc1ncnc(C(=O)N2CCN(c3c4n(c5nc(C6=CCOCC6)nn5c3=O)C(C(=O)Nc3ccc(C(F)(F)F)nc3Cl)CC4C)CC2)c1O ; CC1CC(C(=O)Nc2ccc(C(F)(F)F)cc2Cl)n2c1c(N1CCN(C(=O)c3ncc4c(c3O)OCC4)CC1)c(=O)n1nc(C3=CCOCC3)nc21 ; CC1CC(C(=O)Nc2ccc(C(F)(F)F)cc2Cl)n2c1c(N1CCN(C(=O)c3nccc(F)c3O)CC1)c(=O)n1nc(C3=CCOCC3)nc21]

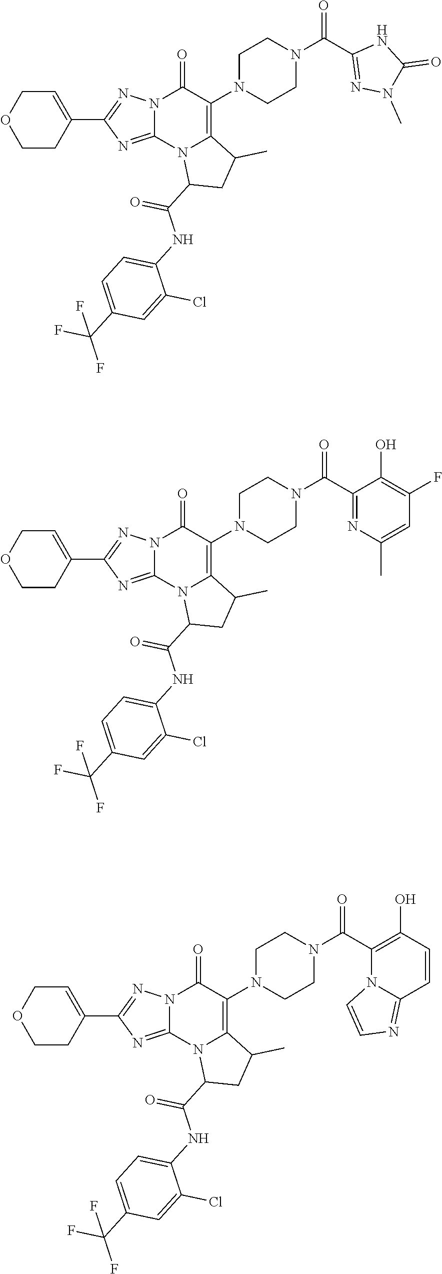 [SMILES: CC1CC(C(=O)Nc2ccc(C(F)(F)F)cc2Cl)n2c1c(N1CCN(C(=O)c3nn(C)c(=O)[nH]3)CC1)c(=O)n1nc(C3=CCOCC3)nc21 ; Cc1cc(F)c(O)c(C(=O)N2CCN(c3c4n(c5nc(C6=CCOCC6)nn5c3=O)C(C(=O)Nc3ccc(C(F)(F)F)cc3Cl)CC4C)CC2)n1 ; CC1CC(C(=O)Nc2ccc(C(F)(F)F)cc2Cl)n2c1c(N1CCN(C(=O)c3c(O)ccc4nccn34)CC1)c(=O)n1nc(C3=CCOCC3)nc21]

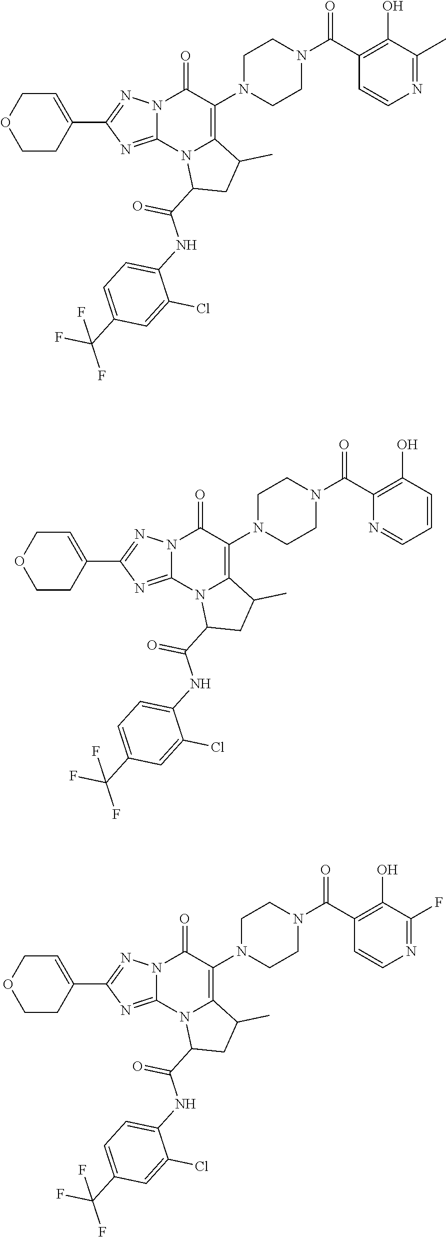 [SMILES: Cc1nccc(C(=O)N2CCN(c3c4n(c5nc(C6=CCOCC6)nn5c3=O)C(C(=O)Nc3ccc(C(F)(F)F)cc3Cl)CC4C)CC2)c1O ; CC1CC(C(=O)Nc2ccc(C(F)(F)F)cc2Cl)n2c1c(N1CCN(C(=O)c3ncccc3O)CC1)c(=O)n1nc(C3=CCOCC3)nc21 ; CC1CC(C(=O)Nc2ccc(C(F)(F)F)cc2Cl)n2c1c(N1CCN(C(=O)c3ccnc(F)c3O)CC1)c(=O)n1nc(C3=CCOCC3)nc21]

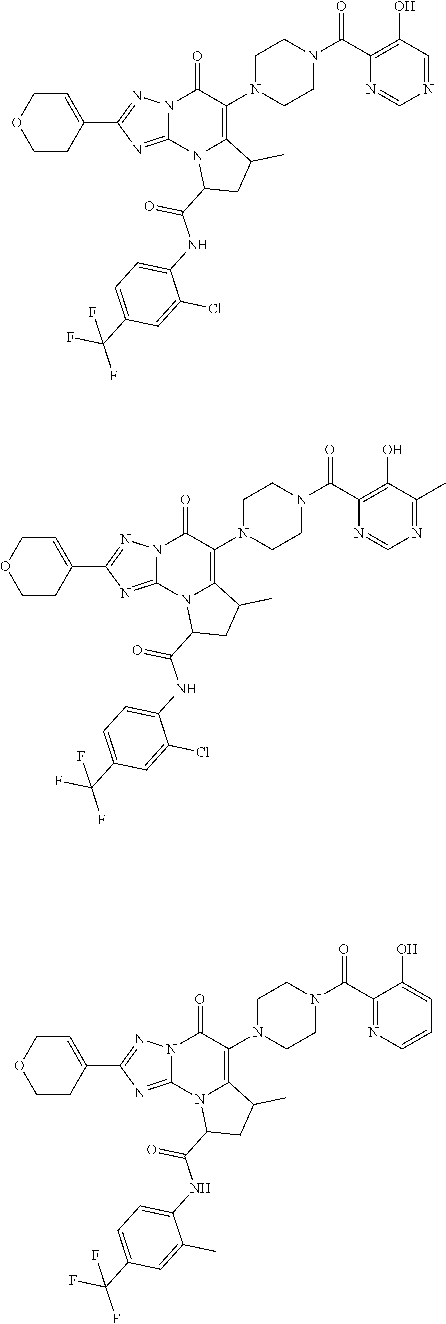 [SMILES: CC1CC(C(=O)Nc2ccc(C(F)(F)F)cc2Cl)n2c1c(N1CCN(C(=O)c3ncncc3O)CC1)c(=O)n1nc(C3=CCOCC3)nc21 ; Cc1ncnc(C(=O)N2CCN(c3c4n(c5nc(C6=CCOCC6)nn5c3=O)C(C(=O)Nc3ccc(C(F)(F)F)cc3Cl)CC4C)CC2)c1O ; Cc1cc(C(F)(F)F)ccc1NC(=O)C1CC(C)c2c(N3CCN(C(=O)c4ncccc4O)CC3)c(=O)n3nc(C4=CCOCC4)nc3n21]

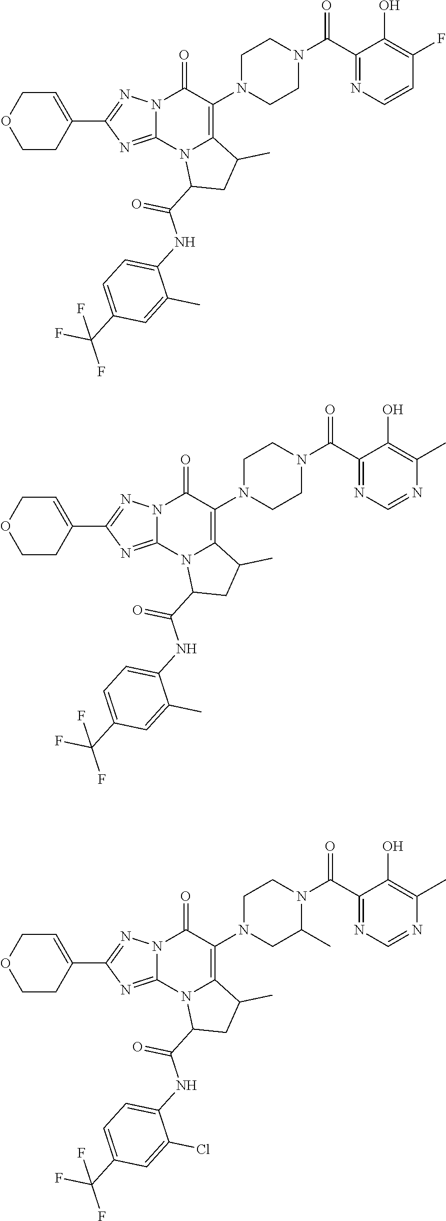 [SMILES: Cc1cc(C(F)(F)F)ccc1NC(=O)C1CC(C)c2c(N3CCN(C(=O)c4nccc(F)c4O)CC3)c(=O)n3nc(C4=CCOCC4)nc3n21 ; Cc1cc(C(F)(F)F)ccc1NC(=O)C1CC(C)c2c(N3CCN(C(=O)c4ncnc(C)c4O)CC3)c(=O)n3nc(C4=CCOCC4)nc3n21 ; Cc1ncnc(C(=O)N2CCN(c3c4n(c5nc(C6=CCOCC6)nn5c3=O)C(C(=O)Nc3ccc(C(F)(F)F)cc3Cl)CC4C)CC2C)c1O]

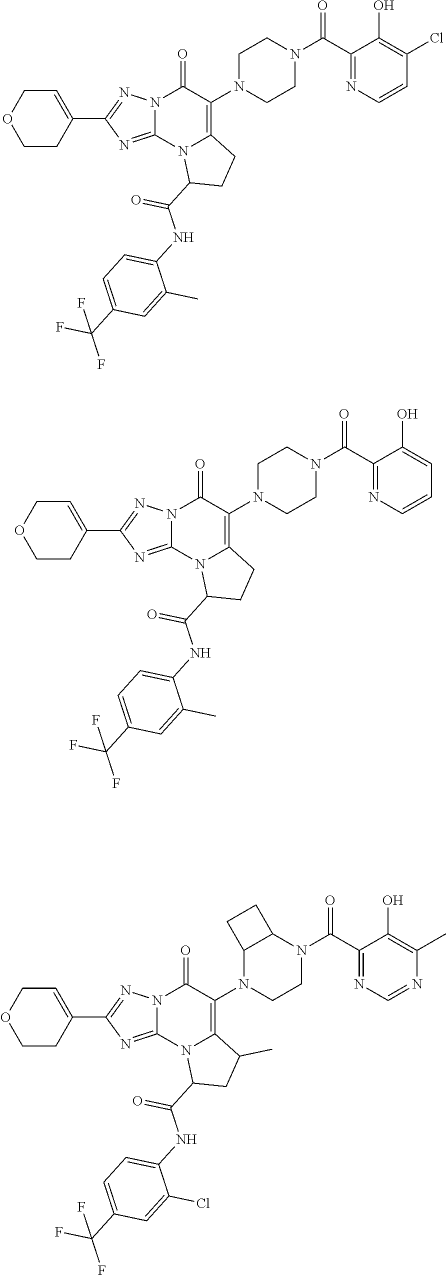 [SMILES: Cc1cc(C(F)(F)F)ccc1NC(=O)C1CCc2c(N3CCN(C(=O)c4nccc(Cl)c4O)CC3)c(=O)n3nc(C4=CCOCC4)nc3n21 ; Cc1cc(C(F)(F)F)ccc1NC(=O)C1CCc2c(N3CCN(C(=O)c4ncccc4O)CC3)c(=O)n3nc(C4=CCOCC4)nc3n21 ; Cc1ncnc(C(=O)N2CCN(c3c4n(c5nc(C6=CCOCC6)nn5c3=O)C(C(=O)Nc3ccc(C(F)(F)F)cc3Cl)CC4C)C3CCC32)c1O]

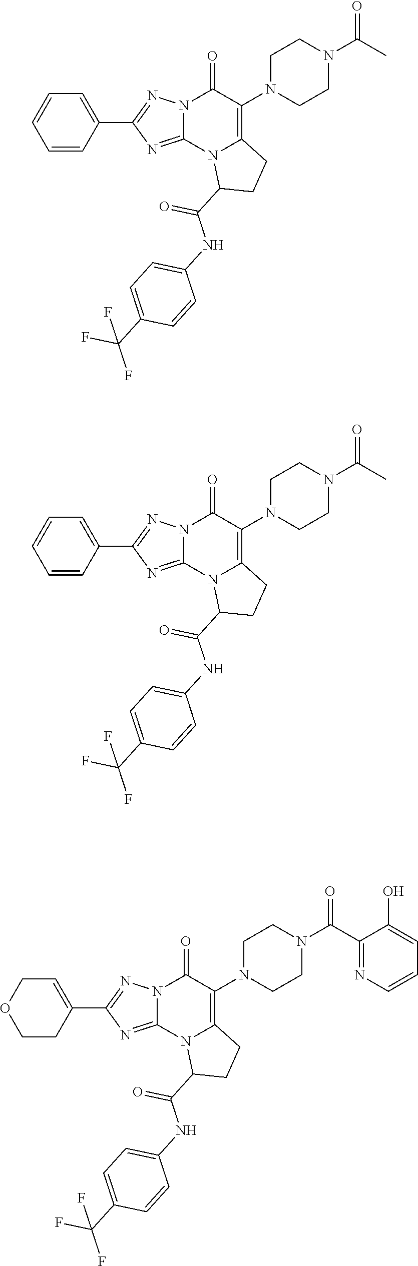 [SMILES: CC(=O)N1CCN(c2c3n(c4nc(-c5ccccc5)nn4c2=O)C(C(=O)Nc2ccc(C(F)(F)F)cc2)CC3)CC1 ; CC(=O)N1CCN(c2c3n(c4nc(-c5ccccc5)nn4c2=O)C(C(=O)Nc2ccc(C(F)(F)F)cc2)CC3)CC1 ; O=C(Nc1ccc(C(F)(F)F)cc1)C1CCc2c(N3CCN(C(=O)c4ncccc4O)CC3)c(=O)n3nc(C4=CCOCC4)nc3n21]

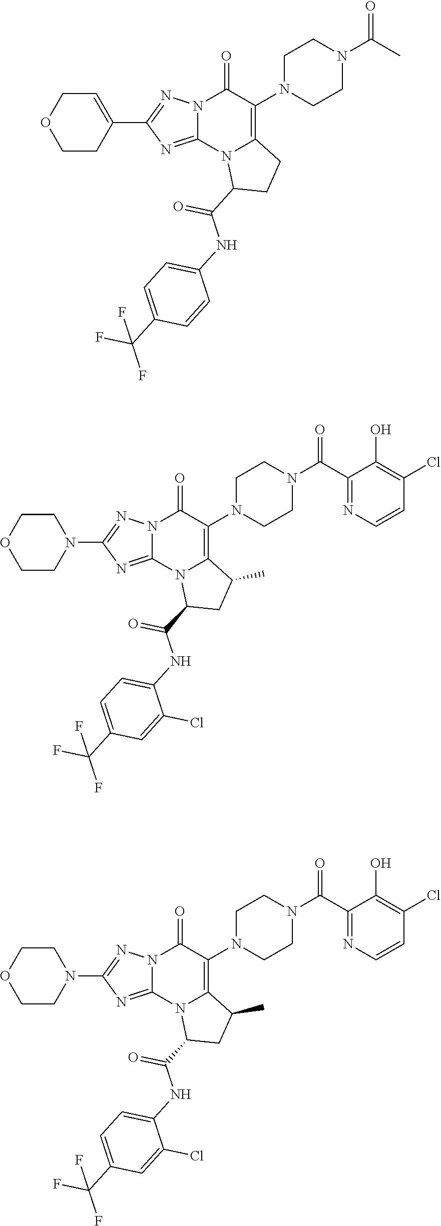 [SMILES: CC(=O)N1CCN(c2c3n(c4nc(C5=CCOCC5)nn4c2=O)C(C(=O)Nc2ccc(C(F)(F)F)cc2)CC3)CC1 ; C[C@@H]1C[C@@H](C(=O)Nc2ccc(C(F)(F)F)cc2Cl)n2c1c(N1CCN(C(=O)c3nccc(Cl)c3O)CC1)c(=O)n1nc(N3CCOCC3)nc21 ; C[C@H]1C[C@H](C(=O)Nc2ccc(C(F)(F)F)cc2Cl)n2c1c(N1CCN(C(=O)c3nccc(Cl)c3O)CC1)c(=O)n1nc(N3CCOCC3)nc21]

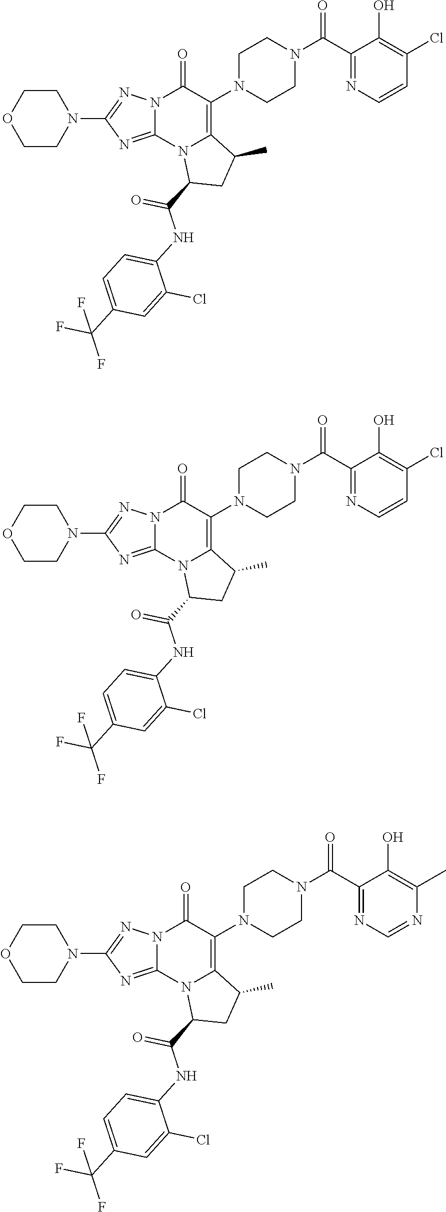 [SMILES: C[C@H]1C[C@@H](C(=O)Nc2ccc(C(F)(F)F)cc2Cl)n2c1c(N1CCN(C(=O)c3nccc(Cl)c3O)CC1)c(=O)n1nc(N3CCOCC3)nc21 ; C[C@@H]1C[C@H](C(=O)Nc2ccc(C(F)(F)F)cc2Cl)n2c1c(N1CCN(C(=O)c3nccc(Cl)c3O)CC1)c(=O)n1nc(N3CCOCC3)nc21 ; Cc1ncnc(C(=O)N2CCN(c3c4n(c5nc(N6CCOCC6)nn5c3=O)[C@H](C(=O)Nc3ccc(C(F)(F)F)cc3Cl)C[C@H]4C)CC2)c1O]

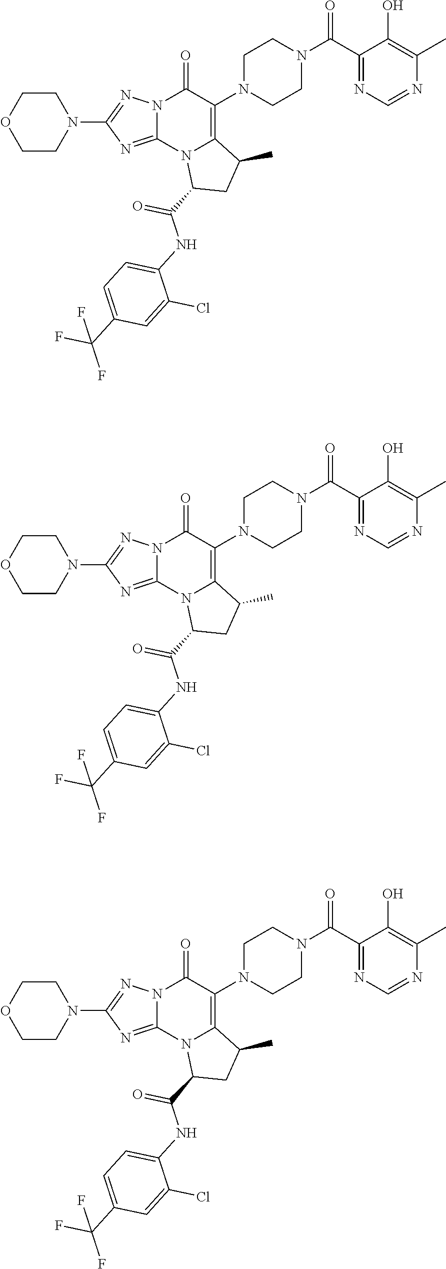 [SMILES: Cc1ncnc(C(=O)N2CCN(c3c4n(c5nc(N6CCOCC6)nn5c3=O)[C@@H](C(=O)Nc3ccc(C(F)(F)F)cc3Cl)C[C@@H]4C)CC2)c1O ; Cc1ncnc(C(=O)N2CCN(c3c4n(c5nc(N6CCOCC6)nn5c3=O)[C@@H](C(=O)Nc3ccc(C(F)(F)F)cc3Cl)C[C@H]4C)CC2)c1O ; Cc1ncnc(C(=O)N2CCN(c3c4n(c5nc(N6CCOCC6)nn5c3=O)[C@H](C(=O)Nc3ccc(C(F)(F)F)cc3Cl)C[C@@H]4C)CC2)c1O]

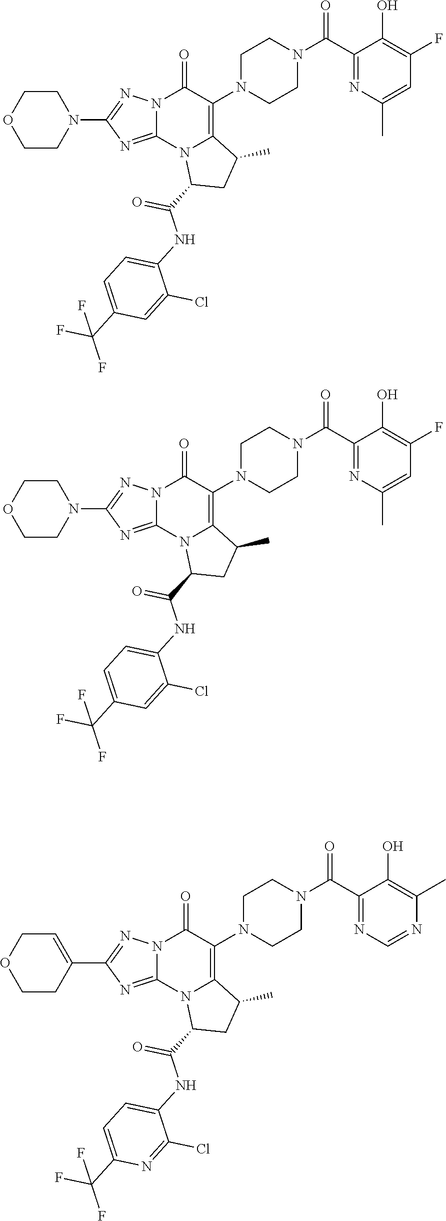 [SMILES: Cc1cc(F)c(O)c(C(=O)N2CCN(c3c4n(c5nc(N6CCOCC6)nn5c3=O)[C@@H](C(=O)Nc3ccc(C(F)(F)F)cc3Cl)C[C@H]4C)CC2)n1 ; Cc1cc(F)c(O)c(C(=O)N2CCN(c3c4n(c5nc(N6CCOCC6)nn5c3=O)[C@H](C(=O)Nc3ccc(C(F)(F)F)cc3Cl)C[C@@H]4C)CC2)n1 ; Cc1ncnc(C(=O)N2CCN(c3c4n(c5nc(C6=CCOCC6)nn5c3=O)[C@@H](C(=O)Nc3ccc(C(F)(F)F)nc3Cl)C[C@H]4C)CC2)c1O]

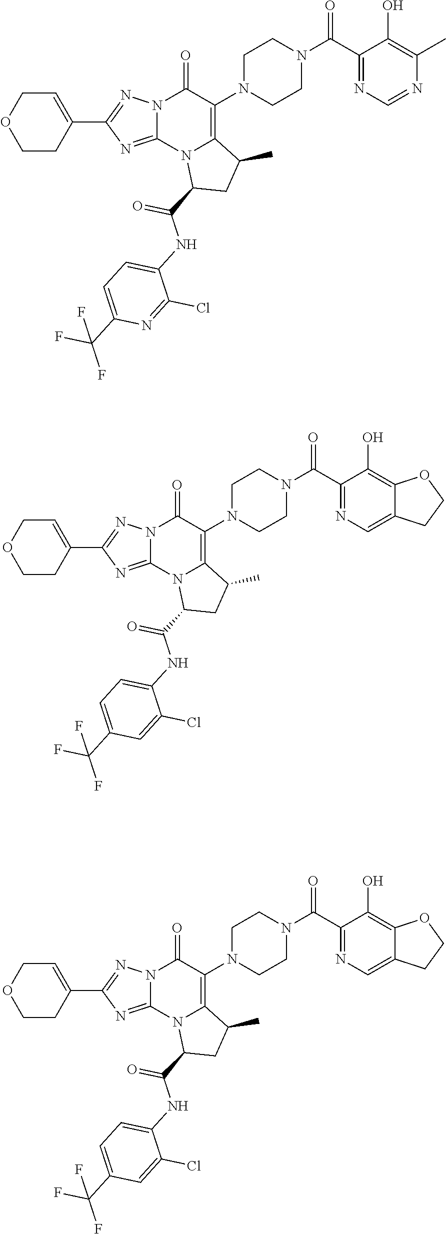 [SMILES: Cc1ncnc(C(=O)N2CCN(c3c4n(c5nc(C6=CCOCC6)nn5c3=O)[C@H](C(=O)Nc3ccc(C(F)(F)F)nc3Cl)C[C@@H]4C)CC2)c1O ; C[C@@H]1C[C@H](C(=O)Nc2ccc(C(F)(F)F)cc2Cl)n2c1c(N1CCN(C(=O)c3ncc4c(c3O)OCC4)CC1)c(=O)n1nc(C3=CCOCC3)nc21 ; C[C@H]1C[C@@H](C(=O)Nc2ccc(C(F)(F)F)cc2Cl)n2c1c(N1CCN(C(=O)c3ncc4c(c3O)OCC4)CC1)c(=O)n1nc(C3=CCOCC3)nc21]

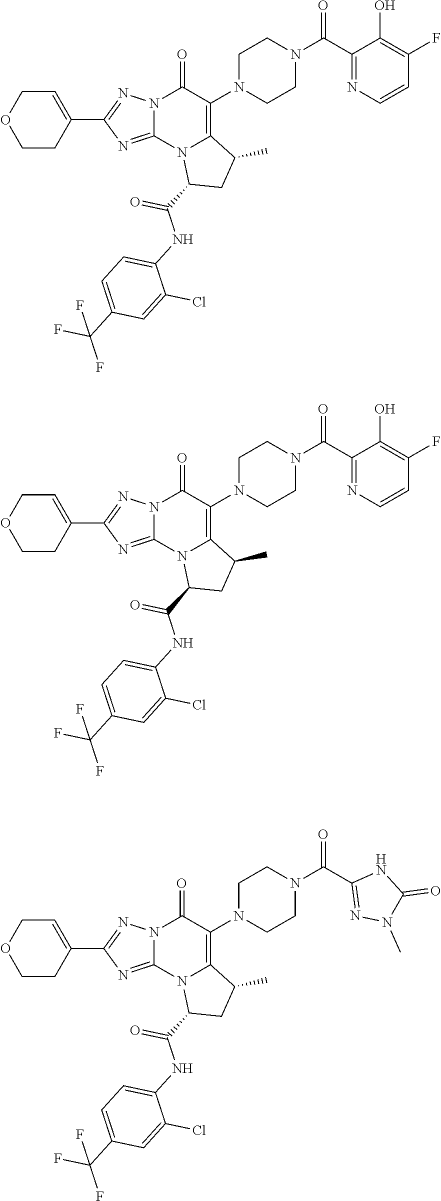 [SMILES: C[C@@H]1C[C@H](C(=O)Nc2ccc(C(F)(F)F)cc2Cl)n2c1c(N1CCN(C(=O)c3nccc(F)c3O)CC1)c(=O)n1nc(C3=CCOCC3)nc21 ; C[C@H]1C[C@@H](C(=O)Nc2ccc(C(F)(F)F)cc2Cl)n2c1c(N1CCN(C(=O)c3nccc(F)c3O)CC1)c(=O)n1nc(C3=CCOCC3)nc21 ; C[C@@H]1C[C@H](C(=O)Nc2ccc(C(F)(F)F)cc2Cl)n2c1c(N1CCN(C(=O)c3nn(C)c(=O)[nH]3)CC1)c(=O)n1nc(C3=CCOCC3)nc21]

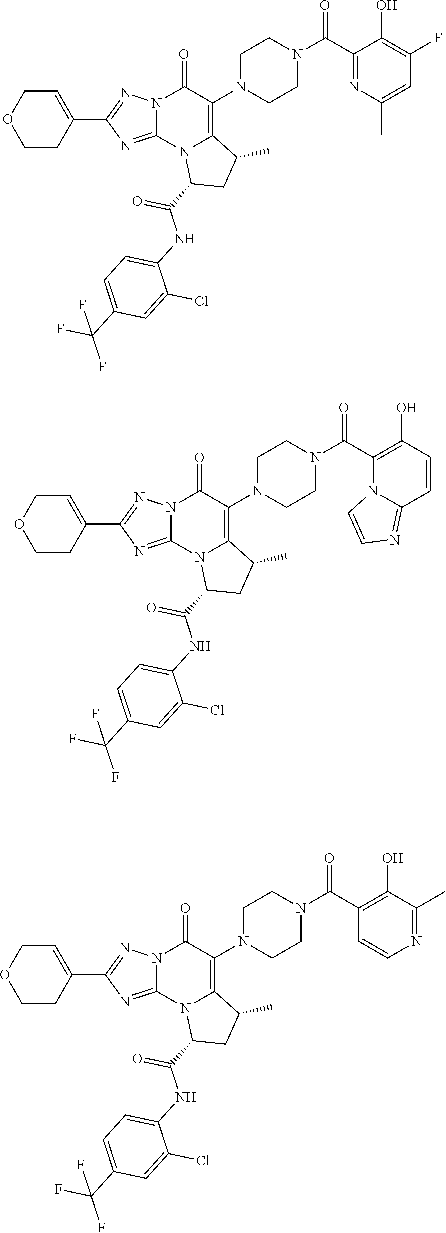 [SMILES: Cc1cc(F)c(O)c(C(=O)N2CCN(c3c4n(c5nc(C6=CCOCC6)nn5c3=O)[C@@H](C(=O)Nc3ccc(C(F)(F)F)cc3Cl)C[C@H]4C)CC2)n1 ; C[C@@H]1C[C@H](C(=O)Nc2ccc(C(F)(F)F)cc2Cl)n2c1c(N1CCN(C(=O)c3c(O)ccc4nccn34)CC1)c(=O)n1nc(C3=CCOCC3)nc21 ; Cc1nccc(C(=O)N2CCN(c3c4n(c5nc(C6=CCOCC6)nn5c3=O)[C@@H](C(=O)Nc3ccc(C(F)(F)F)cc3Cl)C[C@H]4C)CC2)c1O]

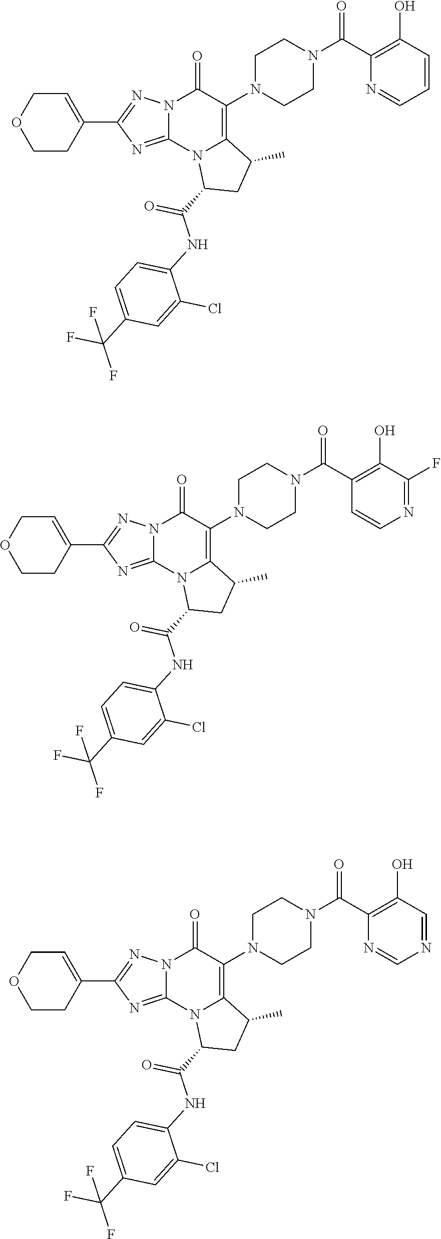 [SMILES: C[C@@H]1C[C@H](C(=O)Nc2ccc(C(F)(F)F)cc2Cl)n2c1c(N1CCN(C(=O)c3ncccc3O)CC1)c(=O)n1nc(C3=CCOCC3)nc21 ; C[C@@H]1C[C@H](C(=O)Nc2ccc(C(F)(F)F)cc2Cl)n2c1c(N1CCN(C(=O)c3ccnc(F)c3O)CC1)c(=O)n1nc(C3=CCOCC3)nc21 ; C[C@@H]1C[C@H](C(=O)Nc2ccc(C(F)(F)F)cc2Cl)n2c1c(N1CCN(C(=O)c3ncncc3O)CC1)c(=O)n1nc(C3=CCOCC3)nc21]

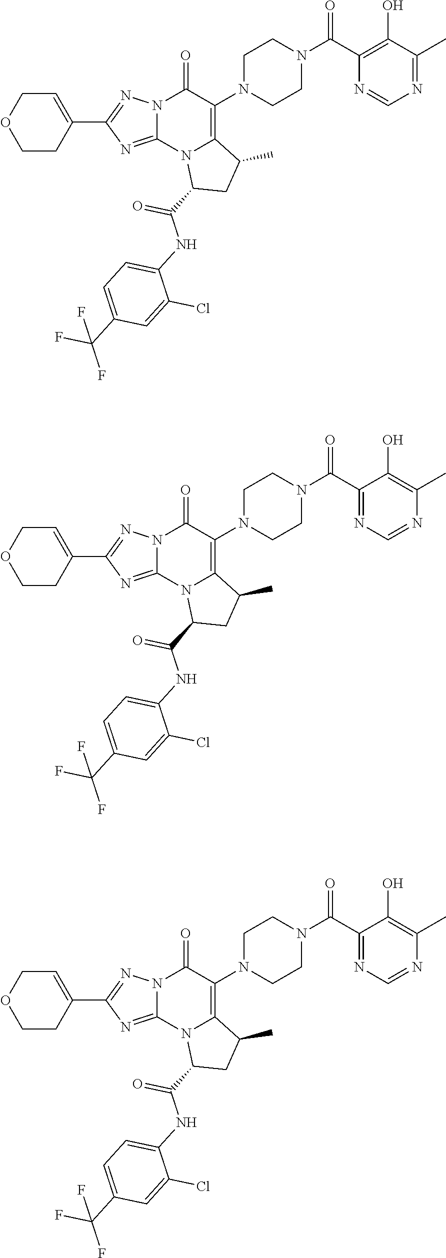 [SMILES: Cc1ncnc(C(=O)N2CCN(c3c4n(c5nc(C6=CCOCC6)nn5c3=O)[C@@H](C(=O)Nc3ccc(C(F)(F)F)cc3Cl)C[C@H]4C)CC2)c1O ; Cc1ncnc(C(=O)N2CCN(c3c4n(c5nc(C6=CCOCC6)nn5c3=O)[C@H](C(=O)Nc3ccc(C(F)(F)F)cc3Cl)C[C@@H]4C)CC2)c1O ; Cc1ncnc(C(=O)N2CCN(c3c4n(c5nc(C6=CCOCC6)nn5c3=O)[C@@H](C(=O)Nc3ccc(C(F)(F)F)cc3Cl)C[C@@H]4C)CC2)c1O]

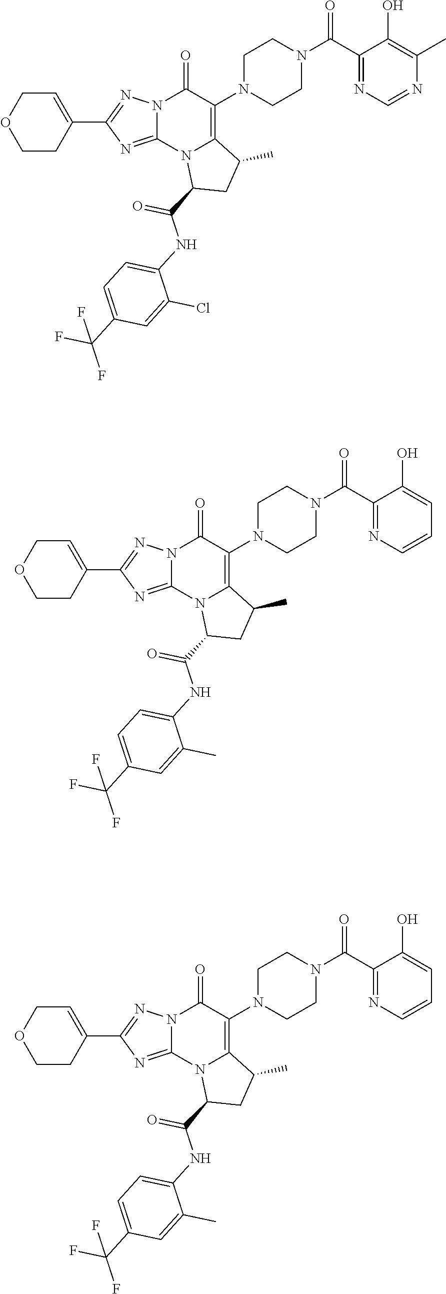 [SMILES: Cc1ncnc(C(=O)N2CCN(c3c4n(c5nc(C6=CCOCC6)nn5c3=O)[C@H](C(=O)Nc3ccc(C(F)(F)F)cc3Cl)C[C@H]4C)CC2)c1O ; Cc1cc(C(F)(F)F)ccc1NC(=O)[C@H]1C[C@H](C)c2c(N3CCN(C(=O)c4ncccc4O)CC3)c(=O)n3nc(C4=CCOCC4)nc3n21 ; Cc1cc(C(F)(F)F)ccc1NC(=O)[C@@H]1C[C@@H](C)c2c(N3CCN(C(=O)c4ncccc4O)CC3)c(=O)n3nc(C4=CCOCC4)nc3n21]

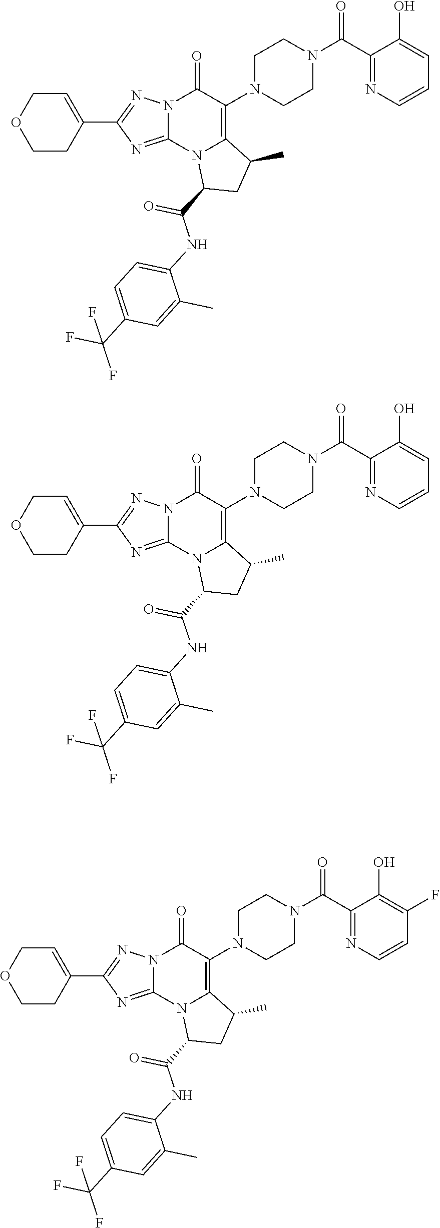 [SMILES: Cc1cc(C(F)(F)F)ccc1NC(=O)[C@@H]1C[C@H](C)c2c(N3CCN(C(=O)c4ncccc4O)CC3)c(=O)n3nc(C4=CCOCC4)nc3n21 ; Cc1cc(C(F)(F)F)ccc1NC(=O)[C@H]1C[C@@H](C)c2c(N3CCN(C(=O)c4ncccc4O)CC3)c(=O)n3nc(C4=CCOCC4)nc3n21 ; Cc1cc(C(F)(F)F)ccc1NC(=O)[C@H]1C[C@@H](C)c2c(N3CCN(C(=O)c4nccc(F)c4O)CC3)c(=O)n3nc(C4=CCOCC4)nc3n21]

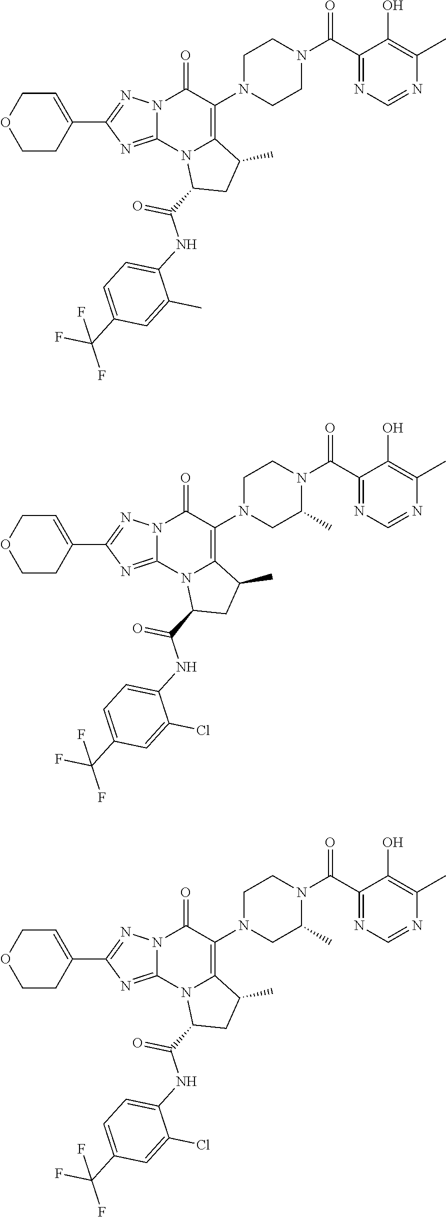 [SMILES: Cc1cc(C(F)(F)F)ccc1NC(=O)[C@H]1C[C@@H](C)c2c(N3CCN(C(=O)c4ncnc(C)c4O)CC3)c(=O)n3nc(C4=CCOCC4)nc3n21 ; Cc1ncnc(C(=O)N2CCN(c3c4n(c5nc(C6=CCOCC6)nn5c3=O)[C@H](C(=O)Nc3ccc(C(F)(F)F)cc3Cl)C[C@@H]4C)C[C@H]2C)c1O ; Cc1ncnc(C(=O)N2CCN(c3c4n(c5nc(C6=CCOCC6)nn5c3=O)[C@@H](C(=O)Nc3ccc(C(F)(F)F)cc3Cl)C[C@H]4C)C[C@H]2C)c1O]

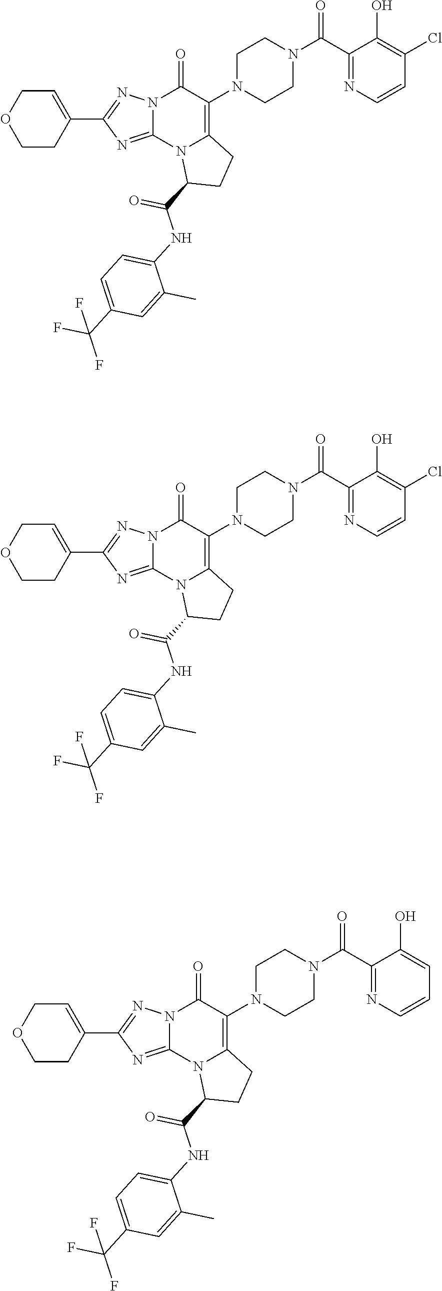 [SMILES: Cc1cc(C(F)(F)F)ccc1NC(=O)[C@@H]1CCc2c(N3CCN(C(=O)c4nccc(Cl)c4O)CC3)c(=O)n3nc(C4=CCOCC4)nc3n21 ; Cc1cc(C(F)(F)F)ccc1NC(=O)[C@H]1CCc2c(N3CCN(C(=O)c4nccc(Cl)c4O)CC3)c(=O)n3nc(C4=CCOCC4)nc3n21 ; Cc1cc(C(F)(F)F)ccc1NC(=O)[C@@H]1CCc2c(N3CCN(C(=O)c4ncccc4O)CC3)c(=O)n3nc(C4=CCOCC4)nc3n21]

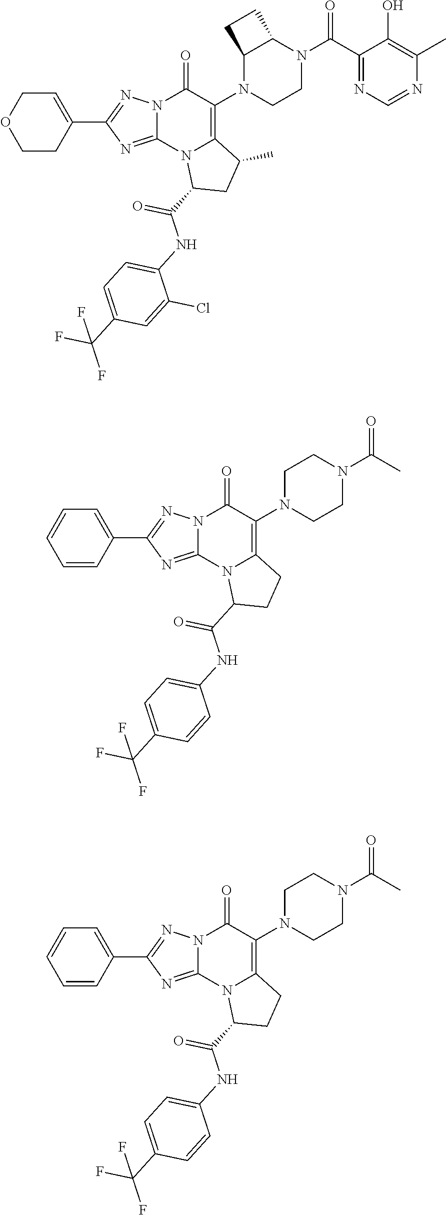 [SMILES: Cc1ncnc(C(=O)N2CCN(c3c4n(c5nc(C6=CCOCC6)nn5c3=O)[C@@H](C(=O)Nc3ccc(C(F)(F)F)cc3Cl)C[C@H]4C)[C@H]3CC[C@@H]32)c1O ; CC(=O)N1CCN(c2c3n(c4nc(-c5ccccc5)nn4c2=O)C(C(=O)Nc2ccc(C(F)(F)F)cc2)CC3)CC1 ; CC(=O)N1CCN(c2c3n(c4nc(-c5ccccc5)nn4c2=O)[C@@H](C(=O)Nc2ccc(C(F)(F)F)cc2)CC3)CC1]

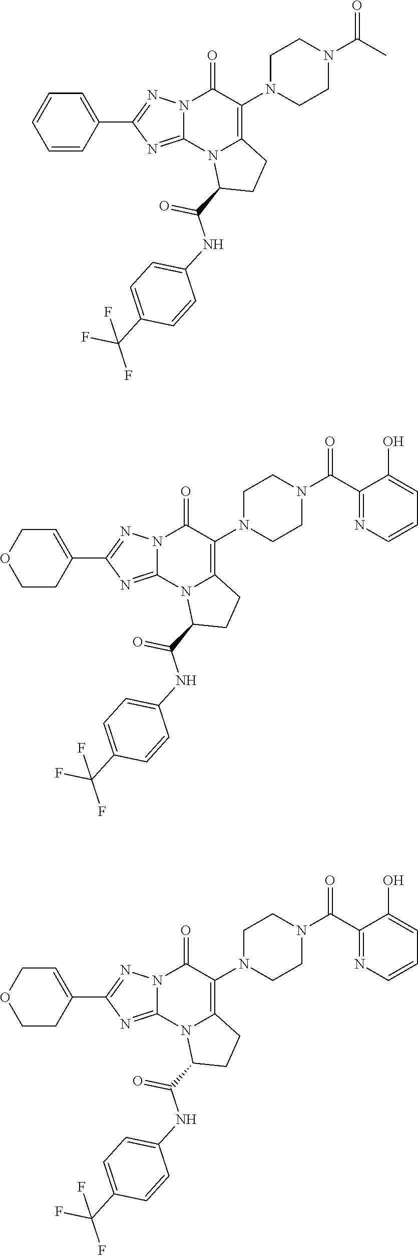 [SMILES: CC(=O)N1CCN(c2c3n(c4nc(-c5ccccc5)nn4c2=O)[C@H](C(=O)Nc2ccc(C(F)(F)F)cc2)CC3)CC1 ; O=C(Nc1ccc(C(F)(F)F)cc1)[C@@H]1CCc2c(N3CCN(C(=O)c4ncccc4O)CC3)c(=O)n3nc(C4=CCOCC4)nc3n21 ; O=C(Nc1ccc(C(F)(F)F)cc1)[C@H]1CCc2c(N3CCN(C(=O)c4ncccc4O)CC3)c(=O)n3nc(C4=CCOCC4)nc3n21]

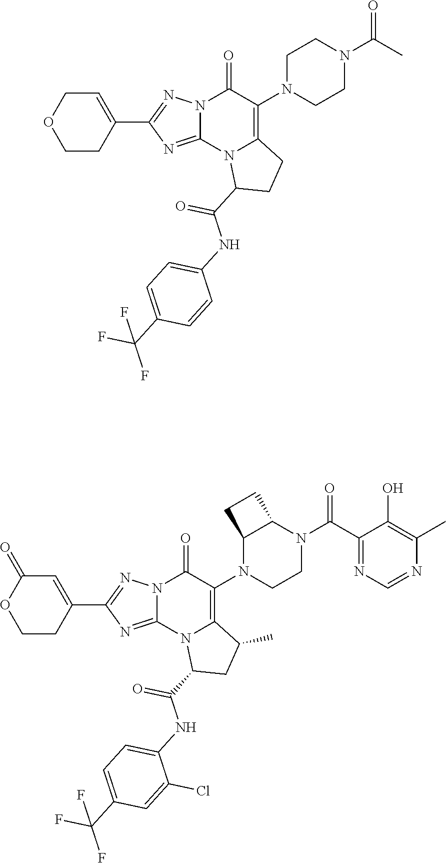 [SMILES: CC(=O)N1CCN(c2c3n(c4nc(C5=CCOCC5)nn4c2=O)C(C(=O)Nc2ccc(C(F)(F)F)cc2)CC3)CC1 ; Cc1ncnc(C(=O)N2CCN(c3c4n(c5nc(C6=CC(=O)OCC6)nn5c3=O)[C@@H](C(=O)Nc3ccc(C(F)(F)F)cc3Cl)C[C@H]4C)[C@H]3CC[C@@H]32)c1O]

33. A compound of formula (I), according to claim 1, wherein the compound is 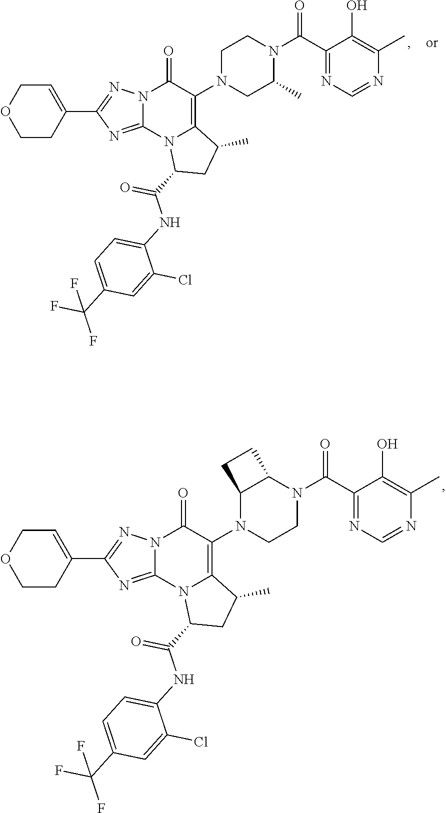 [SMILES: Cc1ncnc(C(=O)N2CCN(c3c4n(c5nc(C6=CCOCC6)nn5c3=O)[C@@H](C(=O)Nc3ccc(C(F)(F)F)cc3Cl)C[C@H]4C)C[C@H]2C)c1O ; Cc1ncnc(C(=O)N2CCN(c3c4n(c5nc(C6=CCOCC6)nn5c3=O)[C@@H](C(=O)Nc3ccc(C(F)(F)F)cc3Cl)C[C@H]4C)[C@H]3CC[C@@H]32)c1O]
or a pharmaceutically acceptable salt thereof.

34. (canceled)

35. A compound of formula (I), according to claim 1, wherein the compound is (7R,9R)—N-(2-chloro-4-(trifluoromethyl)phenyl)-2-(3,6-dihydro-2H-pyran-4-yl)-6-((R)-4-(5-hydroxy-6-methylpyrimidine-4-carbonyl)-3-methylpiperazin-1-yl)-7-methyl-5-oxo-5,7,8,9-tetrahydropyrrolo[1,2-c][1,2,4]triazolo[1,5-a]pyrimidine-9-carboxamide:
in non-zwitterionic form: 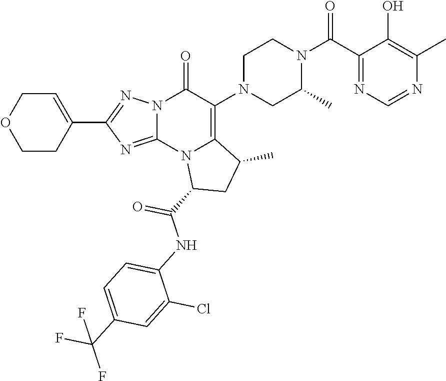 [SMILES: Cc1ncnc(C(=O)N2CCN(c3c4n(c5nc(C6=CCOCC6)nn5c3=O)[C@@H](C(=O)Nc3ccc(C(F)(F)F)cc3Cl)C[C@H]4C)C[C@H]2C)c1O]
or zwitterionic form: 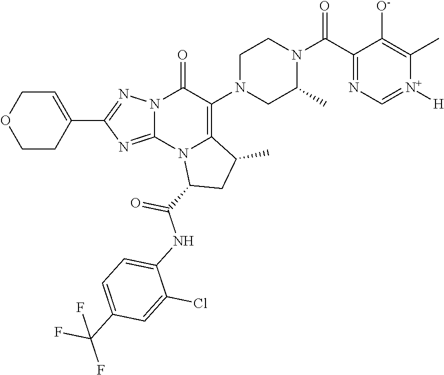 [SMILES: Cc1[nH+]cnc(C(=O)N2CCN(c3c4n(c5nc(C6=CCOCC6)nn5c3=O)[C@@H](C(=O)Nc3ccc(C(F)(F)F)cc3Cl)C[C@H]4C)C[C@H]2C)c1[O-]]
or zwitterionic form: 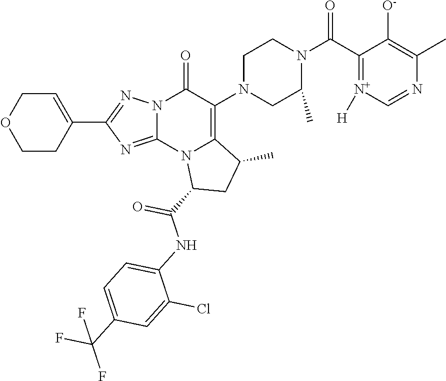 [SMILES: Cc1nc[nH+]c(C(=O)N2CCN(c3c4n(c5nc(C6=CCOCC6)nn5c3=O)[C@@H](C(=O)Nc3ccc(C(F)(F)F)cc3Cl)C[C@H]4C)C[C@H]2C)c1[O-]]
or a mixture of any two or three of said forms.

36. A compound of formula (I), according to claim 1, wherein the compound is (7R,9R)—N-(2-chloro-4-(trifluoromethyl)phenyl)-2-(3,6-dihydro-2H-pyran-4-yl)-6-((1S,6S)-5-(5-hydroxy-6-methylpyrimidine-4-carbonyl)-2,5-diazabicyclo[4.2.0]octan-2-yl)-7-methyl-5-oxo-5,7,8,9-tetrahydropyrrolo[1,2-c][1,2,4]triazolo[1,5-a]pyrimidine-9-carboxamide:
in non-zwitterionic form 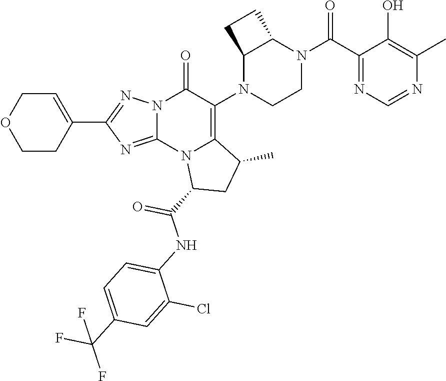 [SMILES: Cc1ncnc(C(=O)N2CCN(c3c4n(c5nc(C6=CCOCC6)nn5c3=O)[C@@H](C(=O)Nc3ccc(C(F)(F)F)cc3Cl)C[C@H]4C)[C@H]3CC[C@@H]32)c1O]
or in zwitterionic form: 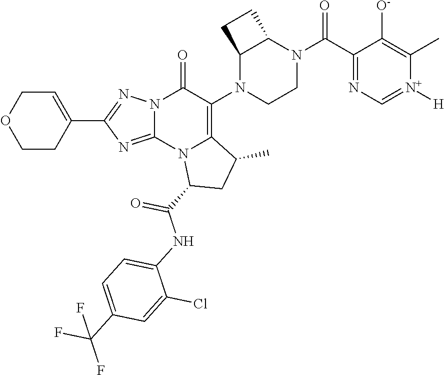 [SMILES: Cc1[nH+]cnc(C(=O)N2CCN(c3c4n(c5nc(C6=CCOCC6)nn5c3=O)[C@@H](C(=O)Nc3ccc(C(F)(F)F)cc3Cl)C[C@H]4C)[C@H]3CC[C@@H]32)c1[O-]]
or in zwitterionic form: 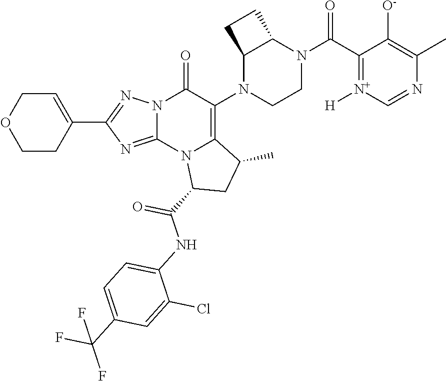 [SMILES: Cc1nc[nH+]c(C(=O)N2CCN(c3c4n(c5nc(C6=CCOCC6)nn5c3=O)[C@@H](C(=O)Nc3ccc(C(F)(F)F)cc3Cl)C[C@H]4C)[C@H]3CC[C@@H]32)c1[O-]]
or a mixture of any two or three of said forms.

37-47. (canceled)

48. A pharmaceutical composition comprising a compound of formula (I) or a pharmaceutically acceptable salt thereof, according to claim 1, and one or more pharmaceutically acceptable carriers.

49-55. (canceled)

56. A method of modulating WRN activity in a subject, wherein the method comprises administering to the subject a therapeutically effective amount of the compound of formula (I), or a pharmaceutically acceptable salt thereof, according to claim 1.

57. A method of inhibiting WRN in a subject, wherein the method comprises administering to the subject a therapeutically effective amount of the compound of formula (I), or a pharmaceutically acceptable salt thereof, according to claim 1.

58. A method of treating a disorder or disease which can be treated by WRN inhibition in a subject, comprising administering to the subject a therapeutically effective amount of the compound of formula (I), or a pharmaceutically acceptable salt thereof, according to claim 1.

59. A method of treating cancer in a subject, comprising administering to the subject a therapeutically effective amount of the compound of formula (I), or a pharmaceutically acceptable salt thereof, according to claim 1.

60. A method of treating cancer in a subject, comprising administering a compound of formula (I), or a pharmaceutically acceptable salt thereof, according to claim 1, wherein the cancer is characterized as microsatellite instability-high (MSI-H) or mismatch repair deficient (dMMR).

61. The method according to claim 60, wherein the cancer characterized as microsatellite instability-high (MSI-H) or mismatch repair deficient (dMMR) is selected from colorectal, gastric, prostate, endometrial, adrenocortical, uterine, cervical, esophageal, breast, kidney and ovarian cancer.

62. (canceled)

63. The method according to claim 60, wherein the cancer characterized as microsatellite instability-high (MSI-H) or mismatch repair deficient (dMMR) is selected from prostate cancer, uterine corpus endometrial carcinoma, colon adenocarcinoma, stomach adenocarcinoma, rectal adenocarcinoma, adrenocortical carcinoma, uterine carcinosarcoma, cervical squamous cell carcinoma, endocervical adenocarcinoma, esophageal carcinoma, breast carcinoma, kidney renal clear cell carcinoma and ovarian serous cystadenocarcinoma.

64-69. (canceled)
